(19) United States
(12) Patent Application Publication
(10) Pub. No.: US 2024/0120032 A1
(43) Pub. Date: Apr. 11, 2024

(54) Crystal Structure of The Human G267s Calpain-5 Protease Core Domain and Its Use in Rational Drug Design for Identifying Inhibitors of Calpain-5

(71) Applicants: The Board of Trustees of the Leland Stanford Junior University, Stanford, CA (US); The United States Government as represented by the Department of Veterans Affairs, Washington, DC (US)

(72) Inventors: Vinit Mahajan, Redwood City, CA (US); Young Joo Sun, Redwood City, CA (US); Gabriel Velez, Redwood City, CA (US); Dylan Parsons, Redwood City, CA (US)

(21) Appl. No.: 18/030,046

(22) PCT Filed: Oct. 14, 2021
(86) PCT No.: PCT/US2021/055026

§ 371 Date: Apr. 3, 2023

Related U.S. Application Data
(60) Provisional application No. 63/091,734, filed on Oct. 14, 2020.

(51) Int. Cl. G16C 20/30; C12N 9/64; G16C 20/50; G16C 20/64

(52) U.S. Cl. CPC G16C 20/30; C12N 9/6472; G16C 20/50; G16C 20/64; C12Y 304/22

(57) ABSTRACT

A high-resolution crystallographic structure of the mutant human G267S calpain-5 protease core domain at 2.22 Å resolution is provided. The G267S mutation is associated with hyperactivity of calpain-5 and is linked to the inherited disease, neovascular inflammatory vitreoretinopathy. Methods of using the crystallographic structure in rational design of small molecule drugs that inhibit calpain-5 for treatment of retinal diseases and other diseases associated with calpain-5 hyperactivity are also provided.

### STATEMENT REGARDING FEDERALLY SPONSORED RESEARCH OR DEVELOPMENT

This invention was made with Government support under contract EY024665 awarded by the National Institutes of Health. The Government has certain rights in the invention.

### INCORPORATION BY REFERENCE OF SEQUENCE LISTING PROVIDED AS A TEXT FILE

A Sequence Listing is provided herewith in a text file, STAN-1795WO_S20-418_ST25, created on Oct. 1, 2021 and having a size of 3,483 bytes. The contents of the text file are incorporated herein by reference in its entirety.

### BACKGROUND OF THE INVENTION

Autosomal dominant neovascular inflammatory vitreoretinopathy (ADNIV) is an inherited disease characterized by retinal synaptic signaling defects, retinal and iris neovascularization, abnormal retinal pigmentation, anterior chamber and vitreous inflammation, cystoid macular edema, vitreous hemorrhage, and retinal detachment. The disease progresses sequentially through a series of pathological stages, each sharing characteristics with common eye diseases (e.g., retinitis pigmentosa, proliferative diabetic retinopathy, and proliferative vitreoretinopathy), ultimately culminating in blindness in the fifth decade. Mutations in the CAPN5 gene cause ADNIV. CAPN5 is the first non-syndromic gene identified as being associated with autoimmune uveitis. CAPN5 encodes a calcium-activated cysteine protease expressed in many tissues, including the central nervous system (CNS) and retinal photoreceptors.

There remains a need for new therapies for treating intraocular inflammation, including ADNIV.

### SUMMARY OF THE INVENTION

A high-resolution crystallographic structure of the mutant human G267S calpain-5 protease core domain at 2.22 Å resolution and methods of crystallizing the mutant human G267S calpain-5 protease core domain are provided. In addition, a computer readable medium comprising atomic coordinates of the x-ray crystallographic structure of the mutant human G267S calpain-5 protease core domain and a computer system comprising atomic coordinates of the x-ray crystallographic structure of the mutant human G267S calpain-5 protease core domain stored in memory are also provided. The G267S mutation is associated with hyperactivity of calpain-5 and is linked to the inherited disease, neovascular inflammatory vitreoretinopathy. Methods of using the crystallographic structure in rational design of small molecule drugs that inhibit calpain-5 for treatment of retinal diseases such as neovascular inflammatory vitreoretinopathy and other diseases associated with calpain-5 hyperactivity are also provided.

In one aspect, a crystal comprising a calpain-5 protease core domain having a G267S mutation is provided, wherein the crystal has P1211 space group symmetry and a unit cell having dimensions of a=84.0 Å, b=51.6 Å, c=110.9 Å, a=90°, p=110.4°, and γ=90°.

In certain embodiments, the calpain-5 protease core domain having the G267S mutation comprises or consists of the amino acid sequence of SEQ ID NO:1 or an amino acid sequence having at least about 80-100% sequence identity to the sequence of SEQ ID NO:1, including any percent identity within this range, such as 81, 82, 83, 84, 85, 86, 87, 88, 89, 90, 91, 92, 93, 94, 95, 96, 97, 98, or 99% sequence identity thereto.

In certain embodiments, X-ray diffraction data collected from the crystal can be used to determine a structure of the calpain-5 protease core domain having the G267S mutation comprising atomic coordinates listed in Table 2±a root mean square deviation of less than 2 Å.

In certain embodiments, the crystal is obtainable by crystallization of the calpain-5 protease core domain having the G267S mutation in a solution comprising or consisting of a buffer (e.g., 100 mM sodium citrate dihydrate) and about 9% to about 11% polyethylene glycol (PEG) 8000 at a pH of about 5.5.

In another aspect, a method of producing a crystal comprising the calpain-5 protease core domain having the G267S mutation is provided, the method comprising crystallizing the calpain-5 protease core domain having the G267S mutation in a crystallization solution comprising or consisting of a buffer (e.g., 100 mM sodium citrate dihydrate) and about 9% to about 11% polyethylene glycol (PEG) 8000 at a pH of about 5.5. In certain embodiments, the crystallization solution further comprises an inhibitor or a substrate of calpain-5.

In certain embodiments, the method further comprises soaking the crystal in a solution (e.g., crystallization solution or stabilization solution) comprising an inhibitor or a substrate of calpain-5 such that the inhibitor or substrate binds to the active site of the calpain-5 protease core domain having the G267S mutation within the crystal.

In another aspect, a crystallographic structure of a crystal comprising the calpain-5 protease core domain having the G267S mutation is provided, wherein the crystallographic structure comprises the atomic coordinates listed in Table 2. In some embodiments, the crystallographic structure has a resolution of 2.2 Å.

In another aspect, a method for identifying a small molecule that binds to the calpain-5 protease core domain having the G267S mutation and inhibits calpain-5 protease activity is provided, the method comprising: a) screening in silico a small molecule library for candidate small molecules likely to bind to the calpain-5 protease core domain using a three-dimensional model of the calpain-5 protease core domain that is computationally derived from the atomic coordinates of the crystallographic structure described herein; and b) evaluating the candidate small molecules identified in step (a) as likely to bind to the calpain-5 protease core domain for their ability to inhibit the calpain-5 variant having a G267S mutation using one or more in vitro or in vivo assays to identify at least one candidate small molecule that inhibits calpain-5 protease activity. In certain embodiments, in step (a), the small molecule library is screened using computational docking for the candidate small molecules, wherein a docking score is calculated for docking of each candidate small molecule in the three-dimensional model of the protease core.

In another aspect, a computer readable medium comprising the atomic coordinates listed in Table 2 is provided.

In another aspect, a method for designing an inhibitor of calpain 5 is provided, the method comprising: a) obtaining a crystal comprising a calpain-5 protease core domain having a G267S mutation, wherein the crystal has P12₁1 space group symmetry and a unit cell having dimensions of a=84.0 Å, b=51.6 Å, c=110.9 Å, α=90°, β=110.4°, and γ=90°; b) determining the three-dimensional structure of the calpain-5 protease core domain having the G267S mutation using the crystal obtained in (a) by X-ray crystallography to obtain the atomic coordinates of the structure; c) providing the atomic coordinates of the three dimensional structure of the calpain-5 protease core domain having the G267S mutation on a computer; and d) utilizing a program operated by the computer to design a chemical compound predicted to bind to the calpain-5 protease core domain having the G267S mutation at a binding location and inhibit protease activity of calpain-5.

In certain embodiments, the binding location is in a substrate binding pocket or the active site within the protease core domain of calpain-5.

In certain embodiments, the designing involves de novo rational drug design.

In certain embodiments, the rational drug design involves (i) identification of functional groups and/or small molecule fragments which can interact with sites in the binding location within the calpain-5 protease core domain, and (ii) linking the functional groups and/or small molecule fragments in a single compound.

In certain embodiments, the designing involves utilizing docking software and screening one or more databases for molecules that fit the binding location within the protease core domain of calpain-5.

In certain embodiments, the method further comprises: synthesizing or obtaining the compound; and evaluating the compound for its ability to perform one or more of (1) binding to calpain-5, (2) competing with a substrate of calpain-5 for binding to the substrate binding site within the calpain-5 protease core domain, and (3) inhibiting protease activity of calpain-5. In some embodiments, the compound binds to the S1 sub-pocket, S2 sub-pocket, S3 sub-pocket, or S4 sub-pocket of the substrate binding site, or any combination thereof. In some embodiments, the rational drug design is based on an interaction between the compound and a residue of the S1 sub-pocket, S2 sub-pocket, S3 sub-pocket, or S4 sub-pocket of the substrate binding site.

In certain embodiments, the rational drug design is based on an interaction between the compound and a residue of the G1 loop, G2 loop, or PC2L2 loop of calpain-5. For example, the rational drug design may be based on an interaction between the compound and a residue at amino acid position 81, 243, 244, 250, 252, 267, 284, or 289 numbered relative to the reference sequence of SEQ ID NO:1.

In another aspect, a computer system comprising the atomic coordinates listed in Table 2 stored in memory is provided.

### BRIEF DESCRIPTION OF THE DRAWINGS

FIGS. 1A-1B. High-resolution structure of CAPN5-PC p.G267S: (FIG. 1A) Ribbon tracing diagram representing domains PC1 and PC2 of the p.G267S human CAPN5 protease core domain at 2.22 Å resolution (CAPN5-PC; light gray). The locations of ADNIV-causing mutations are represented as magenta spheres. Green ribbon, G1 gating loop; pink ribbon, G2 gating loop; orange ribbon PC2L2 loop. (FIG. 1B) Overlay of the wild-type CAPN5-PC (green; 2.83 Å; PDB: 6P3Q) and CAPN5-PC p.G267S (light blue) revealing minimal changes in backbone conformation between wild-type and mutant structures.

FIG. 2. Structural analysis of the CAPN5-PC binding pocket: The 2.22 Å of the p.G267S CAPN5-PC was used to represent the protease core binding sub-pockets and their ligand binding site. Orange; S1 sub-pocket, green; S2 sub-pocket, slate; S3 sub-pocket, and magenta; S4 sub-pocket. Active site residues (Cys81, His252, and Asn284) are denoted by the yellow stick models.

### DETAILED DESCRIPTION OF THE INVENTION

A high-resolution crystallographic structure of the mutant human G267S calpain-5 protease core domain at 2.22 Å resolution and methods of crystallizing the mutant human G267S calpain-5 protease core domain are provided. In addition, a computer readable medium comprising atomic coordinates of the x-ray crystallographic structure of the mutant human G267S calpain-5 protease core domain and a computer system comprising atomic coordinates of the x-ray crystallographic structure of the mutant human G267S calpain-5 protease core domain stored in memory are also provided. The G267S mutation is associated with hyperactivity of calpain-5 and is linked to the inherited disease, neovascular inflammatory vitreoretinopathy. Methods of using the crystallographic structure in rational design of small molecule drugs that inhibit calpain-5 for treatment of retinal diseases such as neovascular inflammatory vitreoretinopathy and other diseases associated with calpain-5 hyperactivity are also provided.

Before the present structure, computer readable/medium, computer system comprising atomic coordinates, and methods of using the crystallographic structure are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. oligopeptides or polypeptides known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “peptide”, “oligopeptide”, “polypeptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

As used herein, the term “binding site” or “binding pocket” refers to a region of a protein or polypeptide (e.g., a calpain-5 protease or a polypeptide fragment thereof comprising a calpain-5 protease core domain) that binds or interacts with a particular compound.

As used herein, the terms “associates with” or “interacts with” refers to a condition of proximity between a chemical entity, compound, or portions thereof, with another chemical entity, compound or portion thereof. The association or interaction may be non-covalent, wherein the juxtaposition is energetically favored by hydrogen bonding or van der Waals or electrostatic interactions, or it may be covalent.

As used herein, the term “pharmacophore” refers to an ensemble of steric and electronic features that is necessary to ensure the optimal supramolecular interactions with a specific biological target structure and to trigger or block a biological response. A pharmacophore may be used to design one or more candidate compounds that comprise all or most of the ensemble of steric and electronic features present in the pharmacophore and that are expected to bind to a site and trigger or block a biological response. Pharmacophores can be used to identify through de novo design or virtual screening novel ligands that will bind to a macromolecule such as a protein (e.g., calpain-5) at a target binding site.

The term “atomic coordinates” refers to the Cartesian coordinates corresponding to an atom's spatial relationship to other atoms in a molecule or molecular complex. Atomic coordinates may be obtained using x-ray crystallography techniques or nuclear magnetic resonance techniques, or may be derived using molecular replacement analysis or homology modeling. Various software programs allow for the graphical representation of a set of structural coordinates to obtain a three-dimensional representation of a molecule or molecular complex. The atomic coordinates of the present disclosure may be modified from the original set provided in Table 2 by mathematical manipulation, such as by inversion or integer additions or subtractions. As such, it is recognized that the structural coordinates of the present invention are relative and are in no way specifically limited by the actual x, y, z coordinates of Table 2.

“Root mean square deviation” is the square root of the arithmetic mean of the squares of the deviations from the mean, and is a way of expressing deviation or variation from the structural coordinates described herein. The present disclosure includes all embodiments comprising conservative substitutions of the noted amino acid residues resulting in same structural coordinates within the stated root mean square deviation. It will be apparent to the skilled practitioner that the numbering of the amino acid residues of the calpain-5 protease or calpain-5 protease core domain may be different than that set forth herein, and may contain certain conservative amino acid substitutions that yield the same three dimensional structures as those defined by Table 2. Corresponding amino acids and conservative substitutions in other isoforms or analogues are easily identified by visual inspection of the relevant amino acid sequences or by using commercially available homology software programs (e.g., MODELLER, Accelrys, San Diego, Calif.; Sali and Blundell (1993) J Mol Biol 234:779-815; Sanchez and Sali (1997) Curr Opin Struct Biol 7: 206-214; and Sanchez and Sali (1998) Proc Natl Acad Sci USA 95: 13597-13602).

The terms “system” and “computer-based system” refer to the hardware means, software means, and data storage means used to analyze the information of the present disclosure. The minimum hardware of the computer-based systems of the present invention comprises a central processing unit (CPU), input means, output means, and data storage means. As such, any convenient computer-based system may be employed in the present disclosure. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid-state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, USB, CD-ROM, DVD, a hard disk drive, flash drive, a ROM or integrated circuit, a magneto-optical disk, or a computer readable card such as a PCMCIA card, SD card, micro SD card, SDHC card, CompactFlash, SmartMedia, Memory Stick, and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e., ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any convenient method. Any convenient data storage structure may be chosen, based on the means used to access the stored information. A variety of data processor programs and formats can be used for storage, e.g., word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

A system can include hardware components which take the form of one or more platforms, e.g., in the form of servers, such that any functional elements of the system, i.e., those elements of the system that carry out specific tasks (such as managing input and output of information, processing information, etc.) of the system may be carried out by the execution of software applications on and across the one or more computer platforms represented of the system. The one or more platforms present in the subject systems may be any convenient type of computer platform, e.g., such as a server, main-frame computer, a work-station, etc. Where more than one platform is present, the platforms may be connected via any convenient type of connection, e.g., cabling or other communication system including wireless systems, either networked or otherwise. Where more than one platform is present, the platforms may be co-located or they may be physically separated. Various operating systems may be employed on any of the computer platforms, where representative operating systems include Windows, MacOS, Sun Solaris, Linux, OS/400, Compaq Tru64 Unix, SGI IRIX, Siemens Reliant Unix, and others. The functional elements of system may also be implemented in accordance with a variety of software facilitators, platforms, or other convenient method.

Items of data are “linked” to one another in a memory when the same data input (for example, filename or directory name or search term) retrieves the linked items (in a same file or not) or an input of one or more of the linked items retrieves one or more of the others.

Subject computer readable media may be at a “remote location”, where “remote location,” means a location other than the location at which the x-ray crystallographic or other analysis is carried out. For example, a remote location could be another location (e.g., office, lab, etc.) in the same city, another location in a different city, another location in a different state, another location in a different country, etc. As such, when one item is indicated as being “remote” from another, what is meant is that the two items may be in the same room but separated, or at least in different rooms or different buildings, and may be at least one mile, ten miles, or at least one hundred miles apart.

“Communicating” information references transmitting the data representing that information as, e.g., electrical or optical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the Internet or Intranets including email transmissions and information recorded on websites and the like.

“Diseases associated with calpain-5” include any disease associated with pathological calpain-5 hyperactivity or overexpression including, without limitation, retinal diseases such as, but not limited to, autosomal neovascular inflammatory vitreoretinopathy (ADNIV), uveitis, retinitis pigmentosa, proliferative diabetic retinopathy, proliferative vitreoretinopathy, and vitreoretinal degeneration.

The term “calpain-5 inhibitor” as used herein refers to any molecule (e.g., small molecule, drug, protein, polypeptide, peptide, peptide mimetic, fusion protein, antibody or fragment thereof, antibody mimetic, or aptamer) that inhibits calpain-5 activity. Inhibition may be complete or partial (i.e., all activity, some activity, or most activity is blocked by an inhibitor). For example, an inhibitor may reduce the activity of calpain-5 by 10%, 20%, 30%, 40%, 50%, 60%, 70%, 80%, 90%, 100%, or any amount in between as compared to native or control levels.

The terms “subject”, “individual” or “patient” are used interchangeably herein and refer to a vertebrate, preferably a mammal. By “vertebrate” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

Crystallographic Structure

A crystallographic structure of a calpain-5 protease core domain having a G267S mutation was determined to 2.2 Å resolution. The structure was determined from a crystal having P12₁1 space group symmetry with a unit cell having dimensions of a=84.0 Å, b=51.6 Å, c=110.9 Å, α=90°, β=110.4°, and γ=90°. The atomic coordinates for the structure of the calpain-5 protease core domain having the G267S mutation are presented in Table 2.

Crystals of the calpain-5 protease core domain having the G267S mutation are obtainable by crystallization of the calpain-5 protease core domain having the G267S mutation in a solution comprising or consisting of about 9% to about 11% polyethylene glycol (PEG) 8000 and a buffer (e.g., 100 mM sodium citrate dihydrate) at a pH of about 5.5. In certain embodiments, the crystallization solution further comprises an inhibitor or a substrate of calpain-5. Temperature may be varied to optimize crystallization. For a description of methods to optimize conditions of crystallization, see, e.g., “Crystallization of Biological Macromolecules” by Alexander McPherson (Cold Spring Harbor Laboratory, 1st edition, Jan. 15, 1999). In some embodiments, crystals are soaked in a solution comprising a cryoprotectant prior to freezing in liquid nitrogen and collection of diffraction data. See Example 1 for a detailed description of crystallization, data collection, and refinement of the structure of the calpain-5 protease core domain having the G267S mutation.

The present disclosure further provides methods for producing a crystal of the calpain-5 protease core domain having the G267S mutation with an inhibitor or a substrate bound at the active site. In some embodiments, the calpain-5 protease core domain is co-crystallized with an inhibitor or a substrate using a precipitating agent (e.g., PEG 8000). In other embodiments, a crystal is soaked in a solution (e.g., crystallization solution or stabilization solution) comprising an inhibitor or a substrate of calpain-5 such that the inhibitor or substrate binds to the active site of the calpain-5 protease core domain having the G267S mutation within the crystal. The inhibitor may be a competitive or a non-competitive inhibitor.

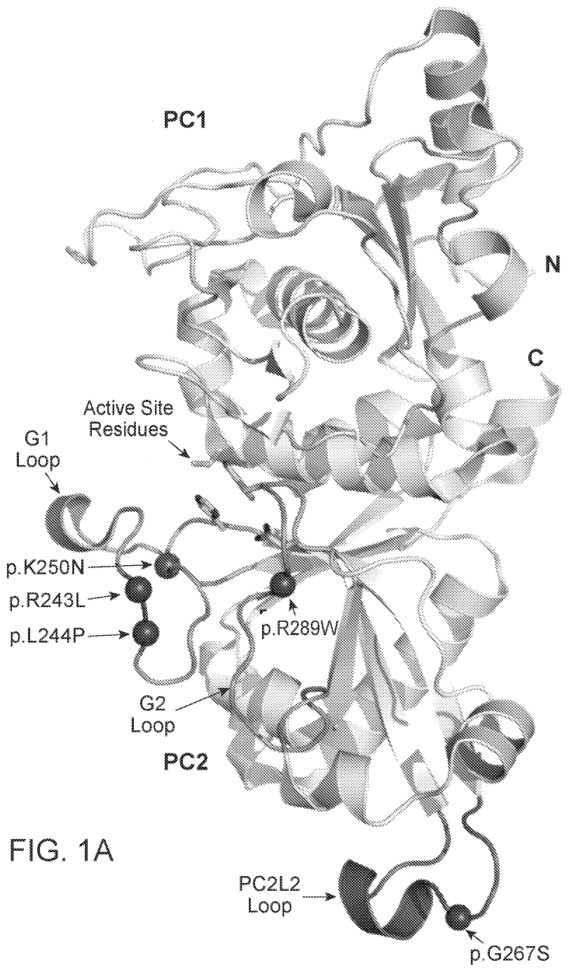

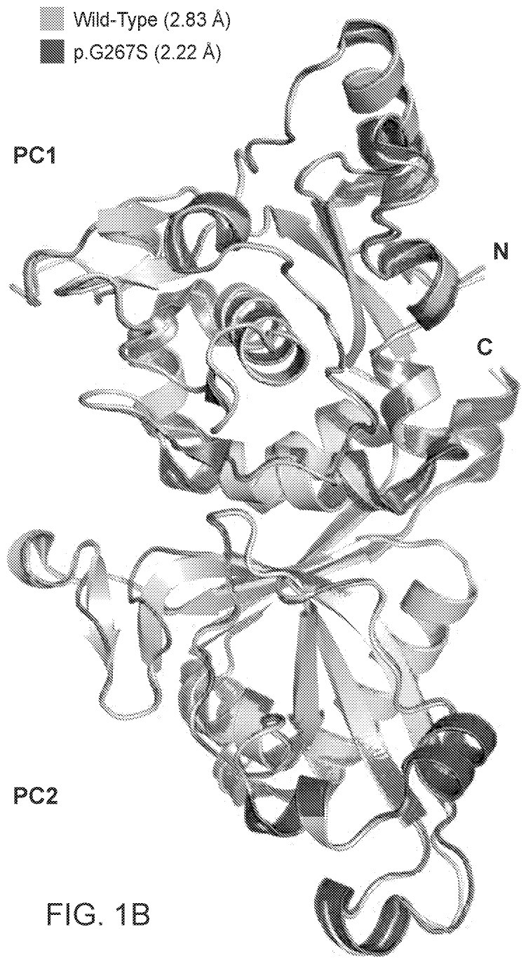

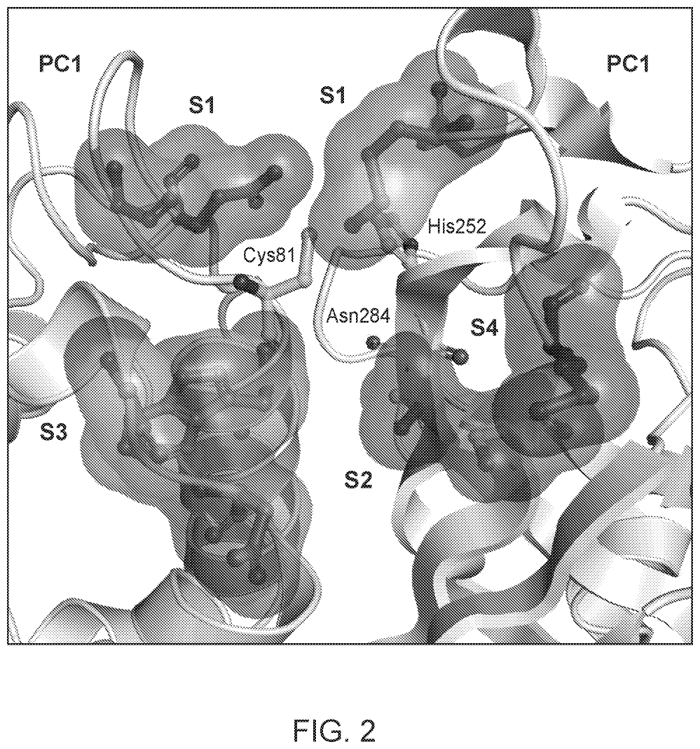

The crystallographic structure provides atomic coordinates for residues of the calpain-5 protease core domain, including, but not limited to, residues of the G1 loop, G2 loop, and PC2L2 loop of calpain-5, residues of the active site, including the catalytic triad residues, Cys81, Asn284, and His252, and the peptide substrate binding site, including residues of the S1 sub-pocket, S2 sub-pocket, S3 sub-pocket, and S4 sub-pocket of the substrate binding site (see FIGS. 1 and 2).

In certain embodiments, the calpain-5 protease core domain having the G267S mutation that is crystallized comprises or consists of the amino acid sequence of SEQ ID NO:1 or an amino acid sequence having at least about 80-100% sequence identity to the sequence of SEQ ID NO:1, including any percent identity within this range, such as 81, 82, 83, 84, 85, 86, 87, 88, 89, 90, 91, 92, 93, 94, 95, 96, 97, 98, or 99% sequence identity thereto, wherein the amino acid sequence at a position corresponding to amino acid 267 of SEQ ID NO: 1 is a serine. The calpain-5 protease core domain having the G267S mutation can be produced using any of a variety of well-known methods, including, e.g., synthetic methods, such as solid phase, liquid phase and combination solid phase/liquid phase syntheses; recombinant DNA methods, including cDNA cloning, optionally combined with site directed mutagenesis; and purification of the polypeptide from a natural source.

Rational Drug Design

The X-ray crystal structure of the calpain-5 protease core domain having the G267S mutation is useful as a model for rationally designing pharmacophores and/or candidate compounds, either de novo or by modification of known compounds. Pharmacophores and candidate compounds identified through the use of the crystal structure coordinates are useful for altering the enzymatic activity and/or substrate selectivity of calpain-5, and so have utility for treating a variety of disorders related to calpain-5 activity. For example, inhibitors of calpain-5 may be used to treat calpain-5-associated diseases including, without limitation, retinal diseases such as, but not limited to, autosomal neovascular inflammatory vitreoretinopathy (ADNIV), uveitis, retinitis pigmentosa, proliferative diabetic retinopathy, proliferative vitreoretinopathy, and vitreoretinal degeneration.

Pharmacophores and candidate compounds may be determined according to any method known in the art. The methods generally involve computationally identifying a compound that binds to calpain-5 (e.g., a compound that binds to a target site such as a substrate-binding site, a catalytic site, or an entrance to the active site of the calpain-5 protease core domain) using the atomic coordinates for the calpain-5 protease core domain having the G267S mutation. For example, in some embodiments, the atomic coordinates are those provided in Table 2. A compound that binds to the calpain-5 protease core domain may include a substrate, a compound that modulates (increases or decreases) enzymatic activity of calpain-5, a compound that modulates substrate specificity/selectivity of calpain-5, or a compound that both modulates enzymatic activity and substrate specificity/selectivity of calpain-5. The compound can be an inhibitor (e.g., an antagonist) or an activator (e.g., an agonist) of protease activity of calpain-5. In some embodiments, the compound is designed de novo. In other embodiments, the compound is designed from a known compound.

In certain embodiments, a method for identifying a small molecule compound that binds to the calpain-5 protease core domain having the G267S mutation is provided, the method comprising screening in silico a small molecule library for candidate small molecules likely to bind to the calpain-5 protease core domain using a three-dimensional model of the calpain-5 protease core domain that is computationally derived from the atomic coordinates of the crystallographic structure of the calpain-5 protease core domain having the G267S mutation. Candidate small molecules, identified as likely to bind to the calpain-5 protease core domain by in silico screening, can be further evaluated using one or more in vitro or in vivo assays to determine their effects on calpain-5 protease activity. In some embodiments, the small molecule library is screened using computational docking for the candidate small molecules, wherein a docking score is calculated for docking of each candidate small molecule in the three-dimensional model of the protease core and used to prioritize candidate small molecules for further screening, as described further below. In certain embodiments, a compound is tested in vivo or in vitro to determine if it binds and/or modulates protease activity or substrate specificity/selectivity of calpain-5. In some embodiments, the method further comprises obtaining the compound (e.g., purchasing or synthesizing the compound) and testing the compound to determine if it modulates (e.g., activates or inhibits) protease activity (e.g., acts an agonist or an antagonist) or substrate specificity/selectivity of calpain-5.

In other embodiments, a subject method involves designing a compound that binds to the calpain-5 protease core domain having the G267S mutation, either de novo, or by modifying an existing compound that is known to bind to the calpain-5 protease core domain. In particular embodiments, a subject method involves computationally identifying a compound that binds to the calpain-5 protease core domain having the G267S mutation using the atomic coordinates set forth in Table 2. In some embodiments, the subject method involves computationally identifying a compound that binds to the S1 sub-pocket, S2 sub-pocket, S3 sub-pocket, or S4 sub-pocket of the substrate binding site, or any combination thereof. For example, rational drug design may be based on an interaction between the compound and one or more residues of the S1 sub-pocket, S2 sub-pocket, S3 sub-pocket, and/or S4 sub-pocket of the substrate binding site. In some embodiments, rational drug design is based on an interaction between the compound and a residue of the G1 loop, G2 loop, or PC2L2 loop of calpain-5. In some embodiments, rational drug design is based on an interaction between the compound and a residue at an amino acid position selected from 81, 243, 244, 250, 252, 267, 284, or 289 numbered relative to the reference sequence of SEQ ID NO:1 as well as those atoms that are in proximity thereto, e.g., within 5 Å, within 10 Å, within 20 Å, or within 30 Å of those amino acids.

In some embodiments, a method of identifying a compound that binds to calpain-5 protease core domain having the G267S mutation (e.g., a compound that binds to a target site such as a substrate-binding site, a catalytic site, or an entrance to the active site of the calpain-5 protease core domain) is provided, the method comprising using a three-dimensional model of the calpain-5 protease core domain that is computationally derived from the atomic coordinates (e.g., listed in Table 2); and evaluating the candidate small molecules identified in step (a) as likely to bind to the calpain-5 protease core domain for their ability to inhibit the calpain-5 variant having a G267S mutation using one or more in vitro or in vivo assays to identify at least one candidate small molecule that inhibits calpain-5 protease activity. In some embodiments, the method further comprises 1) improving the potency of a “lead” compound or a known compound; or 2) designing new compound structures that exhibit improved binding or inhibition of calpain-5 protease activity.

In certain embodiments, a computer system comprising a memory comprising the atomic coordinates of the calpain-5 protease core domain having the G267S mutation is provided. The atomic coordinates are useful as models for rationally identifying compounds that are capable of binding to a target site in the calpain-5 protease core domain. Such compounds may be designed either de novo, or by modification of a known compound. In some cases, binding compounds may be identified by testing known compounds to determine if they “dock” with a molecular model of the calpain-5 protease core domain having the G267S mutation. Such docking methods are well known in the art.

The atomic coordinates of the structure of the calpain-5 protease core domain having the G267S mutation can be used in conjunction with computer-modeling techniques to develop models for in silico screening of binding of various compounds by analysis of the crystal structure data. The structure data provided herein can be used in conjunction with computer-modeling techniques to design compounds that inhibit protease activity of calpain-5. A model of the structure characterizes the three-dimensional topography of a site surface, as well as factors including potential van der Waals contacts, electrostatic interactions, and hydrogen-bonding opportunities. Computer simulation techniques are then used to map interaction positions for functional groups including but not limited to protons, hydroxyl groups, amine groups, divalent cations, aromatic and aliphatic functional groups, amide groups, alcohol groups, etc. that are designed to interact with the model site. These groups may be designed into a pharmacophore or candidate compound with the expectation that the candidate compound will specifically bind to the site. Pharmacophore design thus involves a consideration of the ability of the candidate compounds falling within the pharmacophore to interact with a site through any or all of the available types of chemical interactions, including hydrogen bonding, van der Waals, electrostatic, and covalent interactions, although in general, pharmacophores interact with a site through non-covalent mechanisms.

The ability of a pharmacophore or candidate compound to bind to of the calpain-5 protease core domain having the G267S mutation can be analyzed prior to actual synthesis using computer modeling techniques. Only those candidates that are indicated by computer modeling to bind the target (e.g., a substrate-binding site, a catalytic site, or an entrance to the active site of the calpain-5 protease core domain) with sufficient binding energy (i.e., binding energy corresponding to a dissociation constant with the target on the order of 10-2 M or tighter) may be synthesized and tested for their ability to bind to calpain-5 and inhibit protease activity of calpain-5 using enzyme assays known to those of skill in the art and/or as described herein. The computational evaluation step thus avoids the unnecessary synthesis of compounds that are unlikely to bind to calpain-5 with adequate affinity.

A pharmacophore or candidate compound may be computationally evaluated and designed by means of a series of steps in which chemical entities or fragments are screened and selected for their ability to associate with individual binding target sites on the calpain-5 protease core domain having the G267S mutation. One skilled in the art may use one of several methods to screen chemical entities or fragments for their ability to associate with the calpain-5 protease core domain, and more particularly with target sites on the calpain-5 protease core domain. The process may begin, for example, by visual inspection of a target site on a computer screen based on structural modeling using the calpain-5 protease core domain atomic coordinates, or a subset of those coordinates, as set forth in Table 2.

Selected fragments or chemical entities may then be positioned in a variety of orientations or “docked” within a target site of an the calpain-5 protease core domain as defined from analysis of the crystal structure data. Manual docking may be accomplished using software such as Insight II (Accelrys, San Diego, Calif.) MOE (Chemical Computing Group, Inc., Montreal, Quebec, Canada); and SYBYL (Tripos, Inc., St. Louis, Mo., 1992), followed by energy minimization and/or molecular dynamics with standard molecular mechanics force fields, such as CHARMM (Brooks, et al., J. Comp. Chem. 4:187-217, 1983), AMBER (Weiner, et al., J. Am. Chem. Soc. 106: 765-84, 1984) and C.sup.2 MMFF (Merck Molecular Force Field; Accelrys, San Diego, Calif.). More automated docking may be accomplished by using programs such as DOCK (Kuntz et al., J. Mol. Biol., 161:269-88, 1982; DOCK is available from University of California, San Francisco, Calif.); AUTODOCK (Goodsell & Olsen, Proteins: Structure, Function, and Genetics 8:195-202, 1990; AUTODOCK is available from Scripps Research Institute, La Jolla, Calif.); GOLD (Cambridge Crystallographic Data Centre (CCDC); Jones et al., J. Mol. Biol. 245:43-53, 1995); and FLEXX (Tripos, St. Louis, Mo.; Rarey, M., et al., J. Mol. Biol. 261:470-89, 1996).

Specialized computer programs may also assist in the process of selecting fragments or chemical entities. These include but are not limited to: GRID (Goodford, P. J., “A Computational Procedure for Determining Energetically Favorable Binding Sites on Biologically Important Macromolecules,” J. Med. Chem., 28, pp. 849-857 (1985)); GRID is available from Oxford University, Oxford, UK; MCSS (Miranker, A. and M. Karplus, “Functionality Maps of Binding Sites: A Multiple Copy Simultaneous Search Method,” Proteins: Structure, Function and Genetics, 11, pp. 29-34 (1991)); MCSS is available from Molecular Simulations, Inc., San Diego, Calif.; AUTODOCK (Goodsell, D. S. and A. J. Olsen, “Automated Docking of Substrates to Proteins by Simulated Annealing,” Proteins: Structure, Function, and Genetics, 8, pp. 195-202 (1990)); AUTODOCK is available from Scripps Research Institute, La Jolla, Calif.; DOCK (Kunts, I. D., et al. “A Geometric Approach to Macromolecule-Ligand Interactions,” J. Mol. Biol., 161, pp. 269-288 (1982)); DOCK is available from University of California, San Francisco, Calif.; CERIUS II (available from Accelrys, Inc., San Diego, Calif.); and Flexx (Raret, et al. J. Mol. Biol. 261, pp. 470-489 (1996)).

After selecting suitable chemical entities or fragments, they can be assembled into a single compound. Assembly may proceed by visual inspection of the relationship of the fragments to each other on a three-dimensional image of the fragments in relation to the structure or portion thereof displayed on a computer screen. Visual inspection may be followed by manual model building using software such as the Quanta or Sybyl programs described above.

Software programs also may be used to aid one skilled in the art in connecting the individual chemical entities or fragments. These include, but are not limited to CAVEAT (Bartlett, P. A., et al. “CAVEAT: A Program to Facilitate the Structure-Derived Design of Biologically Active Molecules” In “Molecular Recognition in Chemical and Biological Problems,” Special Publ, Royal Chem. Soc., 78, pp. 182-196 (1989)); CAVEAT is available from the University of California, Berkeley, Calif.; 3D Database systems such as MACCS-3D (MDL Information Systems, San Leandro, Calif.); this area is reviewed in Martin, Y. C., “3D Database Searching in Drug Design,” J. Med. Chem., 35:2145-2154 (1992)); and HOOK (available from Molecular Simulations Inc., San Diego, Calif.).

As an alternative to building candidate pharmacophores or candidate compounds up from individual fragments or chemical entities, they may be designed de novo using the structure of a calpain-5 target site, optionally, including information from co-factor(s), substrates, or known inhibitor(s) that bind to the target site. De novo design may include using programs such as, but not limited to LUDI (Bohm, H. J., “The Computer Program LUDI: A New Method for the De Novo Design of Enzyme Inhibitors, J. Comp. Aid. Molec. Design, 6, pp. 61-78 (1992)); LUDI is available from Molecular Simulations, Inc., San Diego, Calif.; LEGEND (Nishibata, Y., and Rai, A., Tetrahedron 47, p. 8985 (1991); LEGEND is available from Molecular Simulations, San Diego, Calif.; and LeapFrog (available from Tripos Associates, St. Louis, Mo.).

The functional effects of known calpain-5 inhibitors may be altered through the use of molecular modeling and design techniques described herein. This may be carried out by docking the structure of the known calpain-5 inhibitor into the model structure of the calpain-5 protease core domain having the G267S mutation and modifying the structure and charge distribution of the inhibitor to optimize the binding interactions with the calpain-5 protease core domain. The modified structure may be synthesized or obtained from a library of compounds and tested for its binding affinity and/or effect on inhibition of calpain-5 protease activity. This information can be used in optimizing the design of inhibitors. The crystals and structures provided in the present disclosure are especially well suited for methods involving the docking, co-crystallization, structure-based drug design and optimization of inhibitors of calpain-5 protease activity. Molecular, biochemical and computer modeling techniques may be used to design and select novel ligands that interact with calpain-5 and inhibit protease activity of calpain-5. Additional molecular modeling techniques also may be employed. See, e.g., Cohen, N. C., et al. “Molecular Modeling Software and Methods for Medicinal Chemistry,” J. Med. Chem., 33, pp. 883-894 (1990); Navia, M. A. and Murcko, M. A., “The Use of Structural Information in Drug Design,” Curr. Opin. Biotechnol. 8, pp. 696-700 (1997); and Afshar, et al. “Structure-Based and Combinatorial Search for New RNA-Binding Drugs,” Curr. Opin. Biotechnol. 10, pp. 59-63 (1999).

Following pharmacophore or candidate compound design or selection according to any of the above methods or other methods known to one skilled in the art, the efficiency with which a candidate compound falling within the pharmacophore definition binds to the calpain-5 protease core domain having the G267S mutation may be tested and optimized using computational evaluation. A candidate compound may be optimized, e.g., so that in its bound state, it would lack repulsive electrostatic interactions with the target site. Repulsive electrostatic interactions include repulsive charge-charge, dipole-dipole, and charge-dipole interactions. In some embodiments, the sum of all electrostatic interactions between the candidate compound and the calpain-5 protease core domain having the G267S mutation when the candidate compound is bound to the calpain-5 protease core domain make a neutral or favorable contribution to the binding enthalpy.

Specific computer software is available in the art to evaluate compound deformation energy and electrostatic interaction. Examples of programs designed for such uses include: Gaussian 94, revision C (Frisch, Gaussian, Inc., Pittsburgh, Pa. (1995); AMBER, version 7. (Kollman, University of California at San Francisco, (2002); QUANTA/CHARMM (Accelrys, Inc., San Diego, Calif., (1995); Insight II/Discover (Accelrys, Inc., San Diego, Calif., (1995); DelPhi (Accelrys, Inc., San Diego, Calif., (1995); and AMSOL (University of Minnesota) (Quantum Chemistry Program Exchange, Indiana University). These programs may be implemented, for instance, using a computer workstation, as are well known in the art, for example, a LINUX, SGI or Sun workstation. Other hardware systems and software packages will be known to those skilled in the art.

Once a pharmacophore or candidate compound has been optimally selected or designed, as described above, substitutions may then be made in some of its atoms or side groups to improve or modify its binding properties. Generally, initial substitutions are conservative in that the replacement group will have either approximately same size, or overall structure, or hydrophobicity, or charge as the original group. Components known in the art to alter conformation should be avoided in making substitutions. Substituted candidates may be analyzed for efficiency of fit to a target site in the calpain-5 protease core domain having the G267S mutation using the same methods described above.

Once a candidate compound has been identified using any of the methods described above, it can be screened for its effects on the biological activity of calpain-5. Assays for calpain-5 protease activity are known in the art, and any known assay can be used. See, for example, International Patent Application Publication No. WO 2021/072196, herein incorporated by reference in its entirety.

Computer Models, Computer-Readable Media, and Computer Systems

In certain embodiments, representations or models of a three-dimensional structure of the calpain-5 protease core domain having the G267S mutation are provided. A computer model of the structure of the calpain-5 protease core domain having the G267S mutation can be produced using any suitable software program, including, but not limited to, PYMOL, RasMol, Spartan, Molecular Operating Environment, YASARA, or GRASP software. Suitable computer hardware useful for producing an image of the structure are known to those of skill in the art (e.g., a Silicon Graphics Workstation, Linux PC, or Macintosh PC).

Representations or models of a three-dimensional structure of the calpain-5 protease core domain having the G267S mutation in a complex with a compound (e.g., drug, inhibitor, or substrate) can also be determined based on the crystallographic structure provided in the present disclosure, with use of techniques which include molecular replacement or SIR/MIR (single/multiple isomorphous replacement). Methods of molecular replacement are generally known to those of skill in the art (generally described in Brunger, Meth. Enzym. 1997, 276:558-80; Navaza and Saludjian, Meth Enzym 1997, 276, 581-94; Tong and Rossmann, Meth Enzym 1997, 276:594-611; and Bentley, Meth Enzym 1997, 276:611-19, 1997, each of which is incorporated by this reference herein in its entirety) and are performed by a software program including, for example, the Phaser program (McCoy et al., Acta Crystallogr D Biol Crystallogr 2005, 61:458-64; Stroni et al., Acta Crystallogr D Biol Crystallogr 2004, 60:432-38).

Briefly, X-ray diffraction data are collected from the crystal of the calpain-5 protease core domain having the G267S mutation having a bound ligand. The X-ray diffraction data are transformed to calculate a Patterson function. The Patterson function of the crystallized target structure is compared with a Patterson function calculated from a known structure (referred to herein as a search structure). The Patterson function of the crystallized target structure is rotated on the search structure Patterson function to determine the correct orientation of the crystallized target structure in the crystal. The translation function is then calculated to determine the location of the target structure with respect to the crystal axes. Once the crystallized target structure has been correctly positioned in the unit cell, initial phases for the experimental data can be calculated. These phases are necessary for calculation of an electron density map from which structural differences can be observed, and for refinement of the structure. Alternatively, the phases for the diffraction data can be deduced without an initial structural model through the introduction of a heavy element, such as selenium, mercury or the like. Location of the heavy atoms within the structure using their intrinsic anomalous scattering properties permits calculation of the phases for the complete structure. These methods are known to those skilled in the art. The structural features (e.g., amino acid sequence, conserved di-sulfide bonds, α-helices, and 3-strands or (3-sheets) of the search molecule can be related to the crystallized target structure.

As used herein, the term “model” refers to a representation in a tangible medium of the three-dimensional structure of the calpain-5 protease core domain having the G267S mutation with or without a bound ligand. For example, a model can be a representation of the three-dimensional structure in an electronic file, on a computer screen, on a piece of paper (i.e., on a two dimensional medium), and/or as a ball-and-stick figure. Physical three-dimensional models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Accelrys Inc., San Diego, Calif. The phrase “providing a picture of the model” refers to the ability to generate a “hard copy” of the model. Hard copies include both motion and still pictures. Computer screen images and pictures of the model can be visualized in a number of formats including space-filling representations, backbone traces, ribbon diagrams, and electron density maps.

In certain embodiments, a computer readable medium is provided with the calpain-5 protease core domain structural data and/or information stored thereon. As used herein, the phrase “computer readable medium” refers to storage media readable by a computer, which media may be used to store and retrieve data and software programs incorporating computer code. Exemplary computer readable media include floppy disk, CD-ROM, tape, memory (such as flash memory or system memory), a hard drive, a computer readable card such as a PCMCIA card, SD card, micro SD card, SDHC card, CompactFlash, SmartMedia, Memory Stick, and the like.

Thus, the present invention provides a computer readable medium comprising atomic coordinates of the calpain-5 protease core domain having the G267S mutation with or without a ligand bound at a binding site. In some embodiments, the atomic coordinates are those set forth in Table 2. In some embodiments, a subject computer-readable medium further comprises programming for displaying a molecular model of the calpain-5 protease core domain having the G267S mutation with or without a ligand bound at a binding site. In some embodiments, a subject computer-readable medium further comprises programming for identifying a compound that binds to the calpain-5 protease core domain having the G267S mutation. For example, the programming for identifying a compound that binds to the calpain-5 protease core domain having the G267S mutation can comprise a database of structures of known test compounds.

In another embodiment, a computer system is provided having a memory comprising: X-ray crystallographic structure coordinates defining a structure of the calpain-5 protease core domain having the G267S mutation with or without a bound ligand; and a processor in electrical communication with the memory, wherein the processor generates a molecular model having a three dimensional structure representative of the calpain-5 protease core domain having the G267S mutation with or without a bound ligand. The processor can be adapted for identifying a candidate compound having a structure that is capable of binding to the calpain-5 protease core domain having the G267S mutation.

As used herein, the term “computer system” is understood to mean any general or special purpose system which includes a processor in electrical communication with both a memory and at least one input/output device, such as a terminal. Such a system may include, but is not limited to, personal computers, workstations, and mainframes. The processor may be a general-purpose processor or microprocessor or a specialized processor executing programs located in RAM memory. The programs may be placed in RAM from a storage device, such as a disk or preprogrammed ROM memory. The RAM memory in one embodiment is used both for data storage and program execution. The term computer system also embraces systems where the processor and memory reside in different physical entities, but which are in electrical communication by means of a network.

The processor executes a modeling program which accesses data representative of the calpain-5 protease core domain with or without a bound ligand. In addition, the processor also can execute another program, a compound modeling program, which uses the three-dimensional model of the calpain-5 protease core domain having the G267S mutation with or without a bound ligand to identify compounds having a chemical structure that binds to the calpain-5 protease core domain having the G267S mutation. In one embodiment the compound modeling program and the calpain-5 protease core domain structure modeling program are the same program. In another embodiment, the compound modeling program and the calpain-5 protease core domain structure modeling program are different programs, which programs may be stored on the same or different storage medium. For example, the calpain-5 protease core domain structure modeling program may either store the three-dimensional model of the calpain-5 protease core domain having the G267S mutation in a region of memory accessible both to it and to the compound modeling program, or the calpain-5 protease core domain structure modeling program may be written to external storage, such as a disk, CD ROM, DVD, memory card, or magnetic tape for later access by the compound modeling program.

In certain embodiments, a set of atomic coordinates for the crystallographic structure of the calpain-5 protease core domain having the G267S mutation are sent to a remote location and molecular modeling, pharmacophore/candidate compound design, and in silico screening of candidate compounds is performed remotely.

Compound Libraries for Screening

Inhibitors of calpain-5, identified according to the methods described herein, can be provided from libraries of compounds available from a number of sources or may be derived by combinatorial chemistry approaches known in the art. Such libraries include but are not limited to the available Chemical Director, Maybridge, and natural product collections. In an exemplary embodiment, libraries of compounds with known or predicted structures may be docked to a structure of the calpain-5 protease core domain having the G267S mutation.

Utility

Compounds identified using a method as described above are useful, for example, in the treatment of a condition or disorder that is amenable to treatment by inhibiting calpain-5 activity. Such conditions and disorders include any disease associated with pathological calpain-5 hyperactivity or overexpression including, without limitation, retinal diseases such as, but not limited to, autosomal neovascular inflammatory vitreoretinopathy (ADNIV), uveitis, retinitis pigmentosa, proliferative diabetic retinopathy, proliferative vitreoretinopathy, and vitreoretinal degeneration.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-27 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A crystal comprising a calpain-5 protease core domain having a G267S mutation, wherein the crystal has P12₁1 space group symmetry and a unit cell having dimensions of a=84.0 Å, b=51.6 Å, c=110.9 Å, α=90°, β=110.4°, and γ=90°.

2. The crystal of aspect 1, wherein the calpain-5 protease core domain having the G267S mutation comprises or consists of the amino acid sequence of SEQ ID NO:1 or an amino acid sequence having at least 95% identity to the sequence of SEQ ID NO:1.

3. The crystal of aspect 1 or 2, wherein X-ray diffraction data collected from the crystal can be used to determine a structure of the calpain-5 protease core domain having the G267S mutation comprising atomic coordinates listed in Table 2±a root mean square deviation of less than 2 Å.

4. The crystal of any one of aspects 1 to 3, wherein the crystal is obtainable by crystallization of the calpain-5 protease core domain having the G267S mutation in a solution comprising or consisting of about 9% to about 11% polyethylene glycol (PEG) 8000 and a buffer at a pH of about 5.5.

5. The crystal of aspect 4, wherein the buffer is 100 mM sodium citrate dihydrate.

6. A composition comprising the crystal of any one of aspects 1 to 5.

7. The composition of aspect 6, wherein the crystal diffracts x-rays to allow determination of structure coordinates to a resolution of 2.2 Å.

8. A method of producing the crystal of any one of aspects 1 to 5, the method comprising crystallizing the calpain-5 protease core domain having the G267S mutation in a crystallization solution comprising or consisting of about 9% to about 11% polyethylene glycol (PEG) 8000 and a buffer at a pH of about 5.5.

9. The method of aspect 8, wherein the buffer is 100 mM sodium citrate dihydrate.

10. The method of aspect 8 or 9, further comprising soaking the crystal in a solution comprising an inhibitor or a substrate of calpain-5 such that the inhibitor or substrate binds to the active site of the calpain-5 protease core domain having the G267S mutation within the crystal.

11. The method of aspect 8 or 9, wherein the crystallization solution further comprises an inhibitor or a substrate of calpain-5.

12. The crystallographic structure of the crystal of any one of aspects 1 to 5 having the atomic coordinates listed in Table 2.

13. The crystallographic structure of aspect 12, wherein the crystallographic structure has a resolution of 2.2 Å.

14. A method for identifying a small molecule that binds to the calpain-5 protease core domain having the G267S mutation and inhibits calpain-5 protease activity, the method comprising:
a) screening in silico a small molecule library for candidate small molecules likely to bind to the calpain-5 protease core domain using a three-dimensional model of the calpain-5 protease core domain that is computationally derived from the atomic coordinates of the crystallographic structure of aspect 12 or 13; and
b) evaluating the candidate small molecules identified in step (a) as likely to bind to the calpain-5 protease core domain for their ability to inhibit the calpain-5 variant having a G267S mutation using one or more in vitro or in vivo assays to identify at least one candidate small molecule that inhibits calpain-5 protease activity.

15. The method of aspect 14, wherein in step (a), the small molecule library is screened using computational docking for the candidate small molecules, wherein a docking score is calculated for docking of each candidate small molecule in the three-dimensional model of the protease core.

16. A computer readable medium comprising the atomic coordinates listed in Table 2.

17. A method for designing an inhibitor of calpain 5, the method comprising:
a) obtaining a crystal comprising a calpain-5 protease core domain having a G267S mutation, wherein the crystal has P1211 space group symmetry and a unit cell having dimensions of a=84.0 Å, b=51.6 Å, c=110.9 Å, a=90°, (3=110.4°, and γ=90°;
b) determining the three-dimensional structure of the calpain-5 protease core domain having the G267S mutation using the crystal obtained in (a) by X-ray crystallography to obtain atomic coordinates of the structure;
c) providing the atomic coordinates of the three-dimensional structure of the calpain-5 protease core domain having the G267S mutation on a computer; and
d) utilizing a program operated by the computer to design a chemical compound predicted to bind to the calpain-5 protease core domain having the G267S mutation at a binding location and inhibit protease activity of calpain-5.

18. The method of aspect 17, wherein the designing involves de novo rational drug design.

19. The method of aspect 18, wherein the rational drug design involves (i) identification of functional groups and/or small molecule fragments which can interact with sites in the binding location within the calpain-5 protease core domain, and (ii) linking the functional groups and/or small molecule fragments in a single compound.

20. The method of aspect 18 or 19, wherein the designing involves utilizing docking software and screening one or more databases for molecules that fit the binding location within the protease core domain of calpain-5.

21. The method of any one of aspects 17 to 20, further comprising:
obtaining the compound; and
evaluating the compound for (1) binding to calpain-5, (2) competing with a substrate of calpain-5 for binding to the substrate binding site within the calpain-5 protease core domain, or (3) inhibiting protease activity of calpain-5, or any combination thereof.

22. The method of any one of aspects 17 to 21, wherein the binding location is in a substrate binding pocket or the active site within the protease core domain of calpain-5.

23. The method of aspect 22, wherein the compound binds to the S1 sub-pocket, S2 sub-pocket, S3 sub-pocket, or S4 sub-pocket of the substrate binding site, or any combination thereof.

24. The method of aspect 23, wherein the rational drug design is based on an interaction between the compound and a residue of the S1 sub-pocket, S2 sub-pocket, S3 sub-pocket, or S4 sub-pocket of the substrate binding site.

25. The method of any one of aspects 17 to 24, wherein the rational drug design is based on an interaction between the compound and a residue of the G1 loop, G2 loop, or PC2L2 loop of calpain-5.

26. The method of any one of aspects 17 to 25, wherein the residue is at amino acid position 81, 243, 244, 250, 252, 267, 284, or 289 numbered relative to the reference sequence of SEQ ID NO:1.

27. A computer system comprising the atomic coordinates listed in Table 2 stored in memory.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

High-Resolution Structure of Human CAPN5-PC p.G267S

### Introduction

Vitreoretinal degeneration is a common but difficult-to-treat, blinding eye condition, the molecular basis of which is unknown. We linked CAPN5 mutations to an inherited form of vitreoretinal degeneration, implicating Calpain-5 (CAPN5) in the molecular pathways that drive the disease. Autosomal neovascular inflammatory vitreoretinopathy (ADNIV; OMIM 193235) has 5 sequential stages, each of which mimics a common eye disease (e.g., uveitis, retinitis pigmentosa, proliferative diabetic retinopathy, and proliferative vitreoretinopathy) that together account for a significant fraction of visual morbidity and blindness (1). CAPN5 is a calcium-activated, cysteine protease expressed in the central nervous system and photoreceptors. Sixteen calpain family members comprise a set of proteases that cleave subdomains from target proteins to irreversibly change their function (2). The calpain family is relatively well-studied; and calpain hyperactivity is implicated in numerous ocular pathologies, e.g., retinal degeneration, neovascularization, and cataracts. Yet, because CAPN5 is divergent (and so termed a non-classical calpain), less is known regarding its structure and mechanism of action. In the case of the disease-causing CAPN5 mutations that we study, they likely are activated at a lower calcium concentration, rendering the mutant CAPN5s hyperactive. Among other better understood members of the calpain family, such hyperactivity is likewise associated with disease states, including neuronal injury and retinal degeneration.

2.22 Å Crystal Structure of the CAPN5 p.G267S Mutant's Protease Core Domain:

We have determined the crystal structure of the CAPN5-PC p.G267S mutant's protease core domain. Crystals of CAPN5-PC p.G267S grew in 100 mM sodium citrate dihydrate (pH 5.5) and 9-11% polyethylene glycol (PEG) 8000. Crystals diffracted to 2.22 Å and contained two molecules in the asymmetric unit (Table 1). The space group and unit cell parameters were nearly identical to those of wild-type (WT) CAPN5-PC structure that we have previously reported (PDB: 6P3Q), yet the resolution is slightly better (3). The atomic coordinates for the structure of CAPN5-PC p.G267S are listed in Table 2. The structure for CAPN5-PC p.G267S superimposed well onto the structure of WT CAPN5-PC with an RMSD of 0.258 Å (across 624 Ca; FIG. 1), indicating that there were only minor structural differences between the WT and p.G267S mutant under these conditions. At the reported resolution, amino acid sidechains involved in the CAPN5-PC binding pocket are visible (FIG. 2).

The high-resolution structure of CAPN5-PC serves as a template for rational drug design for CAPN5-associated diseases. Scientists and clinicians may use the structure to design and test compounds to identify inhibitors of CAPN5. Identified inhibitors may ultimately be administered to patients as drugs to treat human diseases including inflammatory eye diseases as well as other inflammatory diseases in which CAPN5 plays a role.

### REFERENCES

1. Mahajan V B, Skeie J M, Bassuk A G, Fingert J H, Braun T A, Daggett H T, Folk J C, Sheffield V C, Stone E M. Calpain-5 mutations cause autoimmune uveitis, retinal neovascularization, and photoreceptor degeneration. PLoS Genet. 2012; 8(10):e1003001. Epub 2012/10/12. doi: 10.1371/journal.pgen.1003001. PubMed PMID: 23055945; PMCID: PMC3464205.
2. Campbell R L, Davies P L. Structure-function relationships in calpains. Biochem J. 2012; 447(3):335-51. Epub 2012/10/06. doi: 10.1042/BJ20120921. PubMed PMID: 23035980.
3. Velez G, Sun Y J, Khan S, Yang J, Herrmann J, Chemudupati T, MacLaren RE, Gakhar L, Wakatsuki S, Bassuk A G, Mahajan V B. Structural Insights into the Unique Activation Mechanisms of a Non-classical Calpain and Its Disease-Causing Variants. Cell Rep. 2020; 30(3):881-92 e5. Epub 2020/01/23. doi: 10.1016/j.celrep.2019.12.077. PubMed PMID: 31968260; PMCID: PMC7001764.

TABLE 1
Crystallographic Data and Refinement Statistics

| | CAPN5-PC p.G267S | |
|---|---|---|
| Data collection statistics | | |
| Beam line | SSRL 12-2 | |
| Wavelength (Å) | 1.0000 | |
| Space group | P 1 21 1 | |
| Unit cell dimensions (a, b, c, α, β, γ) | 84.0 Å, 51.6 Å, 110.9 Å, | |
| | 90°, 110.4°, 90° | |
| Resolution range (Å) | 46.3-2.22 | |
| Total reflections | 300,271 | (29,878) |
| Unique reflections | 44,509 | (4,371) |
| Multiplicity | 6.7 | (6.8) |
| Completeness (%) | 99.7 | (99.3) |
| l/σ (I) | 11.1 | (0.73) |
| Wilson B-factor (Å²) | 50.9 | |
| Rₘₑₐₛ | 0.123 | (2.25) |
| CC_{1/2} | 99.9 | (40.4) |
| Refinement statistics | | |
| Resolution (Å) | 2.22 | |
| No. of reflections used in refinement | 44,415 | (4,366) |
| No. of reflections used for R_{free} | 2,219 | (212) |
| R_{work}/R_{free} | 0.244/0.267 | |
| No. of atoms | 5,604 | |
| Protein | 5,604 | |
| Solvent | 0 | |
| Ligands | 0 | |
| B-factors (Å²) | 69.0 | |
| R.M.S.D.^{†} | | |
| Bond length (Å) | 0.37 | |
| Bond angle (degrees) | 0.37 | |
| Ramachandran statistics (%) | | |

| | |
|---|---|
| In preferred regions | 96.0 |
| In allowed regions | 4.0 |
| Outliers | 0.0 |

*The numbers in parentheses are for the highest-resolution shell.
^{†}Root mean square deviation to ideal values.

TABLE 2
Refinement Statistics and Atomic Coordinates
for the Protein Structure of CAPN5-PC p.G267S

RESOLUTION RANGE HIGH (ANGSTROMS): 2.22
RESOLUTION RANGE LOW (ANGSTROMS): 46.29
DATA CUTOFF (SIGMA(F)): NONE
COMPLETENESS FOR RANGE (%): 99.87
NUMBER OF REFLECTIONS: 42273
FREE R VALUE TEST SET SELECTION: RANDOM
R VALUE (WORKING + TEST SET): 0.23851
R VALUE (WORKING SET): 0.23640
FREE R VALUE: 0.27897
FREE R VALUE TEST SET SIZE (%): 5.0
FREE R VALUE TEST SET COUNT: 2213
MEAN B VALUE (OVERALL, A**2): 58.362
OVERALL ANISOTROPIC B VALUE.
B11 (A**2): 3.65
B22 (A**2): −4.03
B33 (A**2): 0.80
B12 (A**2): −0.00
13 (A**2): −0.86
B23 (A**2): 0.00
ESTIMATED OVERALL COORDINATE ERROR.
ESU BASED ON R VALUE (A): 0.287
ESU BASED ON FREE R VALUE (A): 0.231
ESU BASED ON MAXIMUM LIKELIHOOD (A): 0.267
ESU FOR B VALUES BASED ON MAXIMUM LIKELIHOOD (A**2): 12.207
CORRELATION COEFFICIENT FO-FC: 0.946
CORRELATION COEFFICIENT FO-FC FREE: 0.922
RMS DEVIATIONS FROM IDEAL VALUES COUNT RMS WEIGHT
BOND LENGTHS REFINED ATOMS (A): 5724; 0.007; 0.013
BOND LENGTHS OTHERS (A): 5200; 0.001; 0.017
BOND ANGLES REFINED ATOMS (DEGREES): 7762; 1.510; 1.638
BOND ANGLES OTHERS (DEGREES): 11952; 1.285; 1.586
Listing of atomic coordinates for CAPN5-PC p.G267S, including atom number and name, name
and number of the residue, a one-letter code to specify the chain, x, y, and z atomic coordinates,
occupancy, and temperature factor

| | | | | | | | | | | | |
|---|---|---|---|---|---|---|---|---|---|---|---|
| ATOM | 1 | N | CYS | A | 4 | 28.721 | 24.967 | 22.571 | 1.00 | 106.56 | N |
| ATOM | 2 | CA | CYS | A | 4 | 27.413 | 24.378 | 22.139 | 1.00 | 110.76 | C |
| ATOM | 3 | CB | CYS | A | 4 | 26.253 | 25.054 | 22.864 | 1.00 | 108.66 | C |
| ATOM | 4 | SG | CYS | A | 4 | 24.732 | 24.068 | 22.854 | 1.00 | 109.51 | S |
| ATOM | 5 | C | CYS | A | 4 | 27.279 | 24.475 | 20.607 | 1.00 | 110.92 | C |
| ATOM | 6 | O | CYS | A | 4 | 28.311 | 24.296 | 19.921 | 1.00 | 109.21 | O |
| ATOM | 7 | N | VAL | A | 5 | 26.063 | 24.714 | 20.092 | 1.00 | 100.40 | N |
| ATOM | 8 | CA | VAL | A | 5 | 25.748 | 24.844 | 18.636 | 1.00 | 94.06 | C |
| ATOM | 9 | CB | VAL | A | 5 | 24.736 | 23.769 | 18.193 | 1.00 | 91.82 | C |
| ATOM | 10 | CG1 | VAL | A | 5 | 24.393 | 23.887 | 16.714 | 1.00 | 90.29 | C |
| ATOM | 11 | CG2 | VAL | A | 5 | 25.223 | 22.366 | 18.518 | 1.00 | 85.11 | C |
| ATOM | 12 | C | VAL | A | 5 | 25.202 | 26.254 | 18.368 | 1.00 | 89.19 | C |
| ATOM | 13 | O | VAL | A | 5 | 24.182 | 26.609 | 18.986 | 1.00 | 83.42 | O |
| ATOM | 14 | N | LYS | A | 6 | 25.838 | 27.006 | 17.460 | 1.00 | 91.04 | N |
| ATOM | 15 | CA | LYS | A | 6 | 25.489 | 28.412 | 17.104 | 1.00 | 90.58 | C |
| ATOM | 16 | CB | LYS | A | 6 | 26.502 | 28.948 | 16.081 | 1.00 | 97.41 | C |
| ATOM | 17 | CG | LYS | A | 6 | 26.262 | 30.357 | 15.540 | 1.00 | 109.41 | C |
| ATOM | 18 | CD | LYS | A | 6 | 27.089 | 31.455 | 16.198 | 1.00 | 114.81 | C |
| ATOM | 19 | CE | LYS | A | 6 | 27.414 | 32.606 | 15.262 | 1.00 | 113.82 | C |
| ATOM | 20 | NZ | LYS | A | 6 | 28.570 | 33.404 | 15.742 | 1.00 | 109.56 | N |
| ATOM | 21 | C | LYS | A | 6 | 24.054 | 28.437 | 16.578 | 1.00 | 85.79 | C |
| ATOM | 22 | O | LYS | A | 6 | 23.790 | 27.872 | 15.518 | 1.00 | 69.07 | O |
| ATOM | 23 | N | PRO | A | 7 | 23.088 | 29.067 | 17.298 | 1.00 | 83.74 | N |
| ATOM | 24 | CA | PRO | A | 7 | 21.713 | 29.212 | 16.811 | 1.00 | 82.62 | C |
| ATOM | 25 | CB | PRO | A | 7 | 20.984 | 29.964 | 17.941 | 1.00 | 85.00 | C |
| ATOM | 26 | CG | PRO | A | 7 | 21.846 | 29.750 | 19.162 | 1.00 | 86.40 | C |
| ATOM | 27 | CD | PRO | A | 7 | 23.259 | 29.695 | 18.618 | 1.00 | 87.70 | C |
| ATOM | 28 | C | PRO | A | 7 | 21.637 | 30.026 | 15.509 | 1.00 | 77.42 | C |
| ATOM | 29 | O | PRO | A | 7 | 22.254 | 31.066 | 15.454 | 1.00 | 79.47 | O |
| ATOM | 30 | N | TYR | A | 8 | 20.908 | 29.528 | 14.501 | 1.00 | 77.90 | N |
| ATOM | 31 | CA | TYR | A | 8 | 20.761 | 30.168 | 13.166 | 1.00 | 70.98 | C |
| ATOM | 32 | CB | TYR | A | 8 | 20.284 | 29.172 | 12.101 | 1.00 | 65.74 | C |
| ATOM | 33 | CG | TYR | A | 8 | 20.291 | 29.707 | 10.685 | 1.00 | 58.20 | C |
| ATOM | 34 | CD1 | TYR | A | 8 | 21.476 | 30.051 | 10.052 | 1.00 | 54.41 | C |
| ATOM | 35 | CE1 | TYR | A | 8 | 21.486 | 30.543 | 8.753 | 1.00 | 62.48 | C |
| ATOM | 36 | CZ | TYR | A | 8 | 20.293 | 30.698 | 8.058 | 1.00 | 65.01 | C |
| ATOM | 37 | OH | TYR | A | 8 | 20.281 | 31.184 | 6.775 | 1.00 | 72.84 | O |
| ATOM | 38 | CE2 | TYR | A | 3 | 19.100 | 30.361 | 8.674 | 1.00 | 52.26 | C |
| ATOM | 39 | CD2 | TYR | A | 8 | 19.109 | 29.882 | 9.976 | 1.00 | 56.40 | C |
| ATOM | 40 | C | TYR | A | 8 | 19.791 | 31.345 | 13.311 | 1.00 | 73.50 | C |
| ATOM | 41 | O | TYR | A | 8 | 18.637 | 31.128 | 13.786 | 1.00 | 61.98 | O |
| ATOM | 42 | N | GLU | A | 9 | 20.273 | 32.545 | 12.958 | 1.00 | 78.58 | N |
| ATOM | 43 | CA | GLU | A | 9 | 19.512 | 33.819 | 13.015 | 1.00 | 87.28 | C |
| ATOM | 44 | CB | GLU | A | 9 | 18.452 | 33.813 | 11.908 | 1.00 | 89.18 | C |
| ATOM | 45 | CG | GLU | A | 9 | 19.062 | 33.770 | 10.510 | 1.00 | 91.72 | C |
| ATOM | 46 | CD | GLU | A | 9 | 18.108 | 33.489 | 9.356 | 1.00 | 94.50 | C |
| ATOM | 47 | OE1 | GLU | A | 9 | 16.984 | 32.987 | 9.612 | 1.00 | 94.31 | O |
| ATOM | 48 | OE2 | GLU | A | 9 | 18.494 | 33.767 | 8.195 | 1.00 | 83.50 | O |
| ATOM | 49 | C | GLU | A | 9 | 18.971 | 34.003 | 14.444 | 1.00 | 87.18 | C |
| ATOM | 50 | O | GLU | A | 9 | 17.751 | 34.208 | 14.601 | 1.00 | 89.78 | O |
| ATOM | 51 | N | ASP | A | 10 | 19.870 | 33.918 | 15.437 | 1.00 | 87.35 | N |
| ATOM | 52 | CA | ASP | A | 10 | 19.650 | 34.223 | 16.881 | 1.00 | 85.64 | C |
| ATOM | 53 | CB | ASP | A | 10 | 19.651 | 35.732 | 17.159 | 1.00 | 89.28 | C |
| ATOM | 54 | CG | ASP | A | 10 | 20.904 | 36.459 | 16.702 | 1.00 | 94.24 | C |
| ATOM | 55 | OD1 | ASP | A | 10 | 21.772 | 35.800 | 16.102 | 1.00 | 92.34 | O |
| ATOM | 56 | OD2 | ASP | A | 10 | 20.993 | 37.688 | 16.939 | 1.00 | 102.19 | O |
| ATOM | 57 | C | ASP | A | 10 | 18.319 | 33.642 | 17.352 | 1.00 | 78.01 | C |
| ATOM | 58 | O | ASP | A | 10 | 17.566 | 34.385 | 18.002 | 1.00 | 86.14 | O |
| ATOM | 59 | N | GLN | A | 11 | 18.024 | 32.387 | 17.015 | 1.00 | 69.43 | N |
| ATOM | 60 | CA | GLN | A | 11 | 16.730 | 31.751 | 17.369 | 1.00 | 62.23 | C |
| ATOM | 61 | CB | GLN | A | 11 | 15.961 | 31.347 | 16.113 | 1.00 | 60.64 | C |
| ATOM | 62 | CG | GLN | A | 11 | 14.880 | 32.341 | 15.717 | 1.00 | 56.20 | C |
| ATOM | 63 | CD | GLN | A | 11 | 14.063 | 31.884 | 14.528 | 1.00 | 51.23 | C |
| ATOM | 64 | OE1 | GLN | A | 11 | 14.531 | 31.169 | 13.657 | 1.00 | 47.16 | O |
| ATOM | 65 | NE2 | GLN | A | 11 | 12.819 | 32.313 | 14.468 | 1.00 | 51.28 | N |
| ATOM | 66 | C | GLN | A | 11 | 17.015 | 30.576 | 18.297 | 1.00 | 60.98 | C |
| ATOM | 67 | O | GLN | A | 11 | 17.711 | 29.637 | 17.852 | 1.00 | 59.87 | O |
| ATOM | 68 | N | ASN | A | 12 | 16.501 | 30.652 | 19.529 | 1.00 | 59.16 | N |
| ATOM | 69 | CA | ASN | A | 12 | 16.779 | 29.682 | 20.616 | 1.00 | 60.16 | C |
| ATOM | 70 | CB | ASN | A | 12 | 17.102 | 30.381 | 21.933 | 1.00 | 61.39 | C |
| ATOM | 71 | CG | ASN | A | 12 | 17.729 | 29.428 | 22.929 | 1.00 | 67.09 | C |
| ATOM | 72 | OD1 | ASN | A | 12 | 17.067 | 28.532 | 23.455 | 1.00 | 59.66 | O |
| ATOM | 73 | ND2 | ASN | A | 12 | 19.021 | 29.590 | 23.167 | 1.00 | 74.36 | N |
| ATOM | 74 | C | ASN | A | 12 | 15.594 | 28.722 | 20.770 | 1.00 | 60.28 | C |
| ATOM | 75 | O | ASN | A | 12 | 14.585 | 29.109 | 21.388 | 1.00 | 64.37 | O |
| ATOM | 76 | N | TYR | A | 13 | 15.750 | 27.502 | 20.251 | 1.00 | 56.01 | N |
| ATOM | 77 | CA | TYR | A | 13 | 14.723 | 26.433 | 20.228 | 1.00 | 57.68 | C |
| ATOM | 78 | CB | TYR | A | 13 | 15.316 | 25.097 | 19.765 | 1.00 | 53.81 | C |
| ATOM | 79 | CG | TYR | A | 13 | 14.412 | 23.913 | 20.008 | 1.00 | 52.73 | C |
| ATOM | 80 | CD1 | TYR | A | 13 | 13.305 | 23.668 | 19.213 | 1.00 | 49.43 | C |
| ATOM | 81 | CE1 | TYR | A | 13 | 12.469 | 22.590 | 19.450 | 1.00 | 48.52 | C |
| ATOM | 82 | CZ | TYR | A | 13 | 12.725 | 21.742 | 20.508 | 1.00 | 50.49 | C |
| ATOM | 83 | OH | TYR | A | 13 | 11.929 | 20.667 | 20.760 | 1.00 | 49.09 | O |
| ATOM | 84 | CE2 | TYR | A | 13 | 13.815 | 21.975 | 21.322 | 1.00 | 51.42 | C |
| ATOM | 85 | CD2 | TYR | A | 13 | 14.640 | 23.055 | 21.068 | 1.00 | 55.13 | C |
| ATOM | 86 | C | TYR | A | 13 | 14.095 | 26.267 | 21.614 | 1.00 | 58.10 | C |
| ATOM | 87 | O | TYR | A | 13 | 12.853 | 26.316 | 21.717 | 1.00 | 56.32 | O |
| ATOM | 88 | N | SER | A | 14 | 14.930 | 26.025 | 22.626 | 1.00 | 61.44 | N |
| ATOM | 89 | CA | SER | A | 14 | 14.502 | 25.648 | 24.000 | 1.00 | 66.54 | C |
| ATOM | 90 | CB | SER | A | 14 | 15.688 | 25.273 | 24.872 | 1.00 | 68.34 | C |
| ATOM | 91 | OG | SER | A | 14 | 15.398 | 24.119 | 25.652 | 1.00 | 67.76 | O |
| ATOM | 92 | C | SER | A | 14 | 13.681 | 26.797 | 24.601 | 1.00 | 70.21 | C |
| ATOM | 93 | O | SER | A | 14 | 12.584 | 26.514 | 25.153 | 1.00 | 66.10 | O |
| ATOM | 94 | N | ALA | A | 15 | 14.167 | 28.041 | 24.467 | 1.00 | 69.02 | N |
| ATOM | 95 | CA | ALA | A | 15 | 13.468 | 29.265 | 24.937 | 1.00 | 73.78 | C |
| ATOM | 96 | CB | ALA | A | 15 | 14.276 | 30.510 | 24.653 | 1.00 | 72.47 | C |
| ATOM | 97 | C | ALA | A | 15 | 12.087 | 29.346 | 24.272 | 1.00 | 71.55 | C |
| ATOM | 98 | O | ALA | A | 15 | 11.076 | 29.369 | 25.004 | 1.00 | 74.09 | O |
| ATOM | 99 | N | LEU | A | 16 | 12.047 | 29.366 | 22.939 | 1.00 | 64.60 | N |
| ATOM | 100 | CA | LEU | A | 16 | 10.787 | 29.530 | 22.169 | 1.00 | 60.35 | C |
| ATOM | 101 | CB | LEU | A | 16 | 11.119 | 29.503 | 20.679 | 1.00 | 58.88 | C |
| ATOM | 102 | CG | LEU | A | 16 | 11.864 | 30.729 | 20.162 | 1.00 | 59.29 | C |
| ATOM | 103 | CD1 | LEU | A | 16 | 12.437 | 30.469 | 18.778 | 1.00 | 61.44 | C |
| ATOM | 104 | CD2 | LEU | A | 16 | 10.957 | 31.950 | 20.140 | 1.00 | 59.61 | C |
| ATOM | 105 | C | LEU | A | 16 | 9.784 | 28.435 | 22.565 | 1.00 | 60.53 | C |
| ATOM | 106 | O | LEU | A | 16 | 8.627 | 28.782 | 22.888 | 1.00 | 62.73 | O |
| ATOM | 107 | N | ARG | A | 17 | 10.210 | 27.170 | 22.593 | 1.00 | 59.75 | N |
| ATOM | 108 | CA | ARG | A | 17 | 9.328 | 26.024 | 22.947 | 1.00 | 62.16 | C |
| ATOM | 109 | CB | ARG | A | 17 | 10.082 | 24.691 | 22.852 | 1.00 | 57.18 | C |
| ATOM | 110 | CG | ARG | A | 17 | 9.261 | 23.475 | 23.260 | 1.00 | 56.63 | C |
| ATOM | 111 | CD | ARG | A | 17 | 10.050 | 22.180 | 23.242 | 1.00 | 58.77 | C |
| ATOM | 112 | NE | ARG | A | 17 | 11.157 | 22.195 | 24.187 | 1.00 | 63.32 | N |
| ATOM | 113 | CZ | ARG | A | 17 | 12.011 | 21.196 | 24.378 | 1.00 | 63.49 | C |
| ATOM | 114 | NH1 | ARG | A | 17 | 11.907 | 20.082 | 23.675 | 1.00 | 64.40 | N |
| ATOM | 115 | NH2 | ARG | A | 17 | 12.975 | 21.315 | 25.274 | 1.00 | 66.18 | N |
| ATOM | 116 | C | ARG | A | 17 | 8.750 | 26.266 | 24.351 | 1.00 | 67.90 | C |
| ATOM | 117 | O | ARG | A | 17 | 7.519 | 26.132 | 24.520 | 1.00 | 64.52 | O |
| ATOM | 118 | N | ARG | A | 18 | 9.606 | 26.602 | 25.320 | 1.00 | 75.39 | N |
| ATOM | 119 | CA | ARG | A | 18 | 9.206 | 26.831 | 26.735 | 1.00 | 79.95 | C |
| ATOM | 120 | CB | ARG | A | 18 | 10.453 | 27.156 | 27.566 | 1.00 | 91.87 | C |
| ATOM | 121 | CG | ARG | A | 18 | 10.218 | 27.237 | 29.068 | 1.00 | 94.97 | C |
| ATOM | 122 | CD | ARG | A | 18 | 11.512 | 27.341 | 29.859 | 1.00 | 92.18 | C |
| ATOM | 123 | NE | ARG | A | 18 | 11.217 | 27.279 | 31.283 | 1.00 | 98.57 | N |
| ATOM | 124 | CZ | ARG | A | 18 | 10.970 | 26.163 | 31.972 | 1.00 | 100.25 | C |
| ATOM | 125 | NH1 | ARG | A | 18 | 11.005 | 24.979 | 31.380 | 1.00 | 101.45 | N |
| ATOM | 126 | NH2 | ARG | A | 18 | 10.691 | 26.235 | 33.263 | 1.00 | 95.77 | N |
| ATOM | 127 | C | ARG | A | 18 | 8.117 | 27.917 | 26.776 | 1.00 | 75.14 | C |
| ATOM | 128 | O | ARG | I | 18 | 7.065 | 27.657 | 27.395 | 1.00 | 68.46 | O |
| ATOM | 129 | N | ASP | A | 19 | 8.333 | 29.056 | 26.099 | 1.00 | 76.35 | N |
| ATOM | 130 | CA | ASP | A | 19 | 7.362 | 30.189 | 25.985 | 1.00 | 80.95 | C |
| ATOM | 131 | CB | ASP | A | 19 | 7.866 | 31.308 | 25.062 | 1.00 | 85.03 | C |
| ATOM | 132 | CG | ASP | A | 19 | 6.916 | 32.490 | 24.924 | 1.00 | 88.49 | C |
| ATOM | 133 | OD1 | ASP | A | 19 | 6.912 | 33.355 | 25.818 | 1.00 | 103.48 | O |
| ATOM | 134 | OD2 | ASP | A | 19 | 6.179 | 32.537 | 23.928 | 1.00 | 97.72 | O |
| ATOM | 135 | C | ASP | A | 19 | 6.021 | 29.680 | 25.451 | 1.00 | 82.36 | C |
| ATOM | 136 | O | ASP | A | 19 | 4.983 | 30.004 | 26.045 | 1.00 | 96.76 | O |
| ATOM | 137 | N | CYS | A | 20 | 6.049 | 28.926 | 24.353 | 1.00 | 79.92 | N |
| ATOM | 138 | CA | CYS | A | 20 | 4.842 | 28.439 | 23.642 | 1.00 | 75.76 | C |
| ATOM | 139 | CB | CYS | A | 20 | 5.215 | 27.797 | 22.312 | 1.00 | 78.02 | C |
| ATOM | 140 | SG | CYS | A | 20 | 5.560 | 29.006 | 21.010 | 1.00 | 65.26 | S |
| ATOM | 141 | C | CYS | A | 20 | 4.061 | 27.442 | 24.507 | 1.00 | 75.90 | C |
| ATOM | 142 | O | CYS | A | 20 | 2.817 | 27.459 | 24.425 | 1.00 | 74.00 | O |
| ATOM | 143 | N | ARG | A | 21 | 4.754 | 26.579 | 25.259 | 1.00 | 79.45 | N |
| ATOM | 144 | CA | ARG | A | 21 | 4.128 | 25.615 | 26.211 | 1.00 | 84.93 | C |
| ATOM | 145 | CB | ARG | A | 21 | 5.145 | 24.610 | 26.768 | 1.00 | 80.84 | C |
| ATOM | 146 | CG | ARG | A | 21 | 5.493 | 23.476 | 25.815 | 1.00 | 77.82 | C |
| ATOM | 147 | CD | ARG | A | 21 | 6.771 | 22.765 | 26.207 | 1.00 | 74.46 | C |
| ATOM | 148 | NE | ARG | A | 21 | 6.850 | 21.441 | 25.618 | 1.00 | 76.18 | N |
| ATOM | 149 | CZ | ARG | A | 21 | 7.951 | 20.695 | 25.558 | 1.00 | 85.14 | C |
| ATOM | 150 | NH1 | ARG | A | 21 | 9.089 | 21.142 | 26.062 | 1.00 | 85.51 | N |
| ATOM | 151 | NH2 | ARG | A | 21 | 7.912 | 19.500 | 24.987 | 1.00 | 81.63 | N |
| ATOM | 152 | C | ARG | A | 21 | 3.502 | 26.393 | 27.372 | 1.00 | 81.46 | C |
| ATOM | 153 | O | ARG | A | 21 | 2.473 | 25.933 | 27.898 | 1.00 | 78.86 | O |
| ATOM | 154 | N | ARG | A | 22 | 4.129 | 27.505 | 27.765 | 1.00 | 78.65 | N |
| ATOM | 155 | CA | ARG | A | 22 | 3.639 | 28.372 | 28.863 | 1.00 | 88.13 | C |
| ATOM | 156 | CB | ARG | A | 22 | 4.626 | 29.514 | 29.144 | 1.00 | 100.53 | C |
| ATOM | 157 | CG | ARG | A | 22 | 4.432 | 30.215 | 30.484 | 1.00 | 100.86 | C |
| ATOM | 158 | CD | ARG | A | 22 | 3.683 | 31.524 | 30.323 | 1.00 | 103.05 | C |
| ATOM | 159 | NE | ARG | A | 22 | 3.472 | 32.213 | 31.593 | 1.00 | 102.03 | N |
| ATOM | 160 | CZ | ARG | A | 22 | 2.354 | 32.177 | 32.327 | 1.00 | 102.36 | C |
| ATOM | 161 | NH1 | ARG | A | 22 | 1.297 | 31.478 | 31.944 | 1.00 | 96.60 | N |
| ATOM | 162 | NH2 | ARG | A | 22 | 2.297 | 32.848 | 33.463 | 1.00 | 102.71 | N |
| ATOM | 163 | C | ARG | A | 22 | 2.244 | 28.849 | 28.463 | 1.00 | 82.51 | C |
| ATOM | 164 | O | ARG | A | 22 | 1.274 | 28.411 | 29.117 | 1.00 | 85.56 | O |
| ATOM | 165 | N | ARG | A | 23 | 2.139 | 29.593 | 27.356 | 1.00 | 72.61 | N |
| ATOM | 166 | CA | ARG | A | 23 | 0.879 | 30.277 | 26.942 | 1.00 | 71.02 | C |
| ATOM | 167 | CB | ARG | A | 23 | 1.150 | 31.420 | 25.962 | 1.00 | 74.82 | C |
| ATOM | 168 | CG | ARG | A | 23 | 2.473 | 32.141 | 26.169 | 1.00 | 74.36 | C |
| ATOM | 169 | CD | ARG | A | 23 | 3.013 | 32.564 | 24.821 | 1.00 | 75.46 | C |
| ATOM | 170 | NE | ARG | A | 23 | 3.029 | 34.002 | 24.662 | 1.00 | 77.37 | N |
| ATOM | 171 | CZ | ARG | A | 23 | 3.143 | 34.646 | 23.505 | 1.00 | 84.50 | C |
| ATOM | 172 | NH1 | ARG | A | 23 | 3.221 | 33.983 | 22.361 | 1.00 | 77.41 | N |
| ATOM | 173 | NH2 | ARG | A | 23 | 3.167 | 35.970 | 23.501 | 1.00 | 89.23 | N |
| ATOM | 174 | C | ARG | A | 23 | −0.109 | 29.323 | 26.251 | 1.00 | 64.81 | C |
| ATOM | 175 | O | ARG | A | 23 | −1.154 | 29.828 | 25.802 | 1.00 | 58.29 | O |
| ATOM | 176 | N | LYS | A | 24 | 0.202 | 28.024 | 26.162 | 1.00 | 67.48 | N |
| ATOM | 177 | CA | LYS | A | 24 | −0.519 | 27.004 | 25.341 | 1.00 | 72.93 | C |
| ATOM | 178 | CB | LYS | A | 24 | −1.727 | 26.474 | 26.114 | 1.00 | 74.61 | C |
| ATOM | 179 | CG | LYS | A | 24 | −1.356 | 25.779 | 27.419 | 1.00 | 82.69 | C |
| ATOM | 180 | CD | LYS | A | 24 | −2.539 | 25.509 | 28.326 | 1.00 | 85.97 | C |
| ATOM | 181 | CE | LYS | A | 24 | −2.138 | 24.930 | 29.666 | 1.00 | 83.58 | C |
| ATOM | 182 | NZ | LYS | A | 24 | −2.982 | 25.472 | 30.756 | 1.00 | 84.13 | N |
| ATOM | 183 | C | LYS | A | 24 | −0.860 | 27.582 | 23.956 | 1.00 | 74.32 | C |
| ATOM | 184 | O | LYS | A | 24 | −2.043 | 27.570 | 23.554 | 1.00 | 69.33 | O |
| ATOM | 185 | N | VAL | A | 25 | 0.167 | 28.049 | 23.245 | 1.00 | 75.20 | N |
| ATOM | 186 | CA | VAL | A | 25 | 0.086 | 28.458 | 21.812 | 1.00 | 80.90 | C |
| ATOM | 187 | CB | VAL | A | 25 | 0.528 | 29.926 | 21.644 | 1.00 | 88.36 | C |
| ATOM | 188 | CG1 | VAL | A | 25 | 2.027 | 30.096 | 21.833 | 1.00 | 90.05 | C |
| ATOM | 189 | CG2 | VAL | A | 25 | 0.081 | 30.524 | 20.314 | 1.00 | 89.91 | C |
| ATOM | 190 | C | VAL | A | 25 | 0.926 | 27.476 | 20.970 | 1.00 | 74.34 | C |
| ATOM | 191 | O | VAL | A | 25 | 1.701 | 26.674 | 21.543 | 1.00 | 68.70 | O |
| ATOM | 192 | N | LEU | A | 26 | 0.742 | 27.493 | 19.651 | 1.00 | 62.46 | N |
| ATOM | 193 | CA | LEU | A | 26 | 1.594 | 26.737 | 18.699 | 1.00 | 56.85 | C |
| ATOM | 194 | CB | LEU | A | 26 | 0.716 | 25.999 | 17.684 | 1.00 | 50.31 | C |
| ATOM | 195 | CG | LEU | A | 26 | −0.024 | 24.811 | 18.281 | 1.00 | 47.31 | C |
| ATOM | 196 | CD1 | LEU | A | 26 | −0.704 | 23.984 | 17.199 | 1.00 | 45.15 | C |
| ATOM | 197 | CD2 | LEU | A | 26 | 0.932 | 23.959 | 19.107 | 1.00 | 48.25 | C |
| ATOM | 198 | C | LEU | A | 26 | 2.550 | 27.721 | 18.032 | 1.00 | 51.81 | C |
| ATOM | 199 | O | LEU | A | 26 | 2.076 | 28.749 | 17.514 | 1.00 | 49.39 | O |
| ATOM | 200 | N | PHE | A | 27 | 3.847 | 27.421 | 18.085 | 1.00 | 45.32 | N |
| ATOM | 201 | CA | PHE | A | 27 | 4.910 | 28.334 | 17.603 | 1.00 | 50.10 | C |
| ATOM | 202 | CB | PHE | A | 27 | 6.277 | 27.665 | 17.758 | 1.00 | 46.08 | C |
| ATOM | 203 | CG | PHE | A | 27 | 7.404 | 28.403 | 17.092 | 1.00 | 47.20 | C |
| ATOM | 204 | CD1 | PHE | A | 27 | 7.783 | 29.661 | 17.537 | 1.00 | 51.85 | C |
| ATOM | 205 | CE1 | PHE | A | 27 | 8.819 | 30.351 | 16.921 | 1.00 | 48.43 | C |
| ATOM | 206 | CZ | PHE | A | 27 | 9.487 | 29.774 | 15.861 | 1.00 | 50.83 | C |
| ATOM | 207 | CD2 | PHE | A | 27 | 8.076 | 27.843 | 16.014 | 1.00 | 44.03 | C |
| ATOM | 208 | CE2 | PHE | A | 27 | 9.124 | 28.520 | 15.415 | 1.00 | 46.25 | C |
| ATOM | 209 | C | PHE | A | 27 | 4.565 | 28.747 | 16.166 | 1.00 | 51.96 | C |
| ATOM | 210 | O | PHE | A | 27 | 4.067 | 27.891 | 15.408 | 1.00 | 49.09 | O |
| ATOM | 211 | N | GLU | A | 28 | 4.773 | 30.026 | 15.841 | 1.00 | 59.69 | N |
| ATOM | 212 | CA | GLU | A | 28 | 4.692 | 30.591 | 14.468 | 1.00 | 58.26 | C |
| ATOM | 213 | CB | GLU | A | 28 | 3.514 | 31.549 | 14.316 | 1.00 | 56.15 | C |
| ATOM | 214 | CG | GLU | A | 28 | 2.160 | 30.916 | 14.581 | 1.00 | 63.04 | C |
| ATOM | 215 | CD | GLU | A | 28 | 0.973 | 31.766 | 14.135 | 1.00 | 67.65 | C |
| ATOM | 216 | OE1 | GLU | A | 28 | −0.188 | 31.405 | 14.462 | 1.00 | 69.88 | O |
| ATOM | 217 | OE2 | GLU | A | 28 | 1.205 | 32.795 | 13.458 | 1.00 | 64.69 | O |
| ATOM | 218 | C | GLU | A | 28 | 6.007 | 31.317 | 14.186 | 1.00 | 59.89 | C |
| ATOM | 219 | O | GLU | A | 28 | 6.229 | 32.394 | 14.769 | 1.00 | 65.90 | O |
| ATOM | 220 | N | ASP | A | 29 | 6.856 | 30.713 | 13.357 | 1.00 | 55.84 | N |
| ATOM | 221 | CA | ASP | A | 29 | 8.179 | 31.246 | 12.949 | 1.00 | 57.50 | C |
| ATOM | 222 | CB | ASP | A | 29 | 8.909 | 30.217 | 12.083 | 1.00 | 59.41 | C |
| ATOM | 223 | CG | ASP | A | 29 | 10.349 | 30.567 | 11.757 | 1.00 | 56.43 | C |
| ATOM | 224 | OD1 | ASP | A | 29 | 10.816 | 31.628 | 12.224 | 1.00 | 53.16 | O |
| ATOM | 225 | OD2 | ASP | A | 29 | 10.973 | 29.786 | 11.016 | 1.00 | 51.56 | O |
| ATOM | 226 | C | ASP | A | 29 | 7.989 | 32.579 | 12.222 | 1.00 | 57.59 | C |
| ATOM | 227 | O | ASP | A | 29 | 7.336 | 32.617 | 11.184 | 1.00 | 62.03 | O |
| ATOM | 228 | N | PRO | I | 30 | 8.532 | 33.707 | 12.747 | 1.00 | 55.05 | N |
| ATOM | 229 | CA | PRO | A | 30 | 8.564 | 34.977 | 12.013 | 1.00 | 50.51 | C |
| ATOM | 230 | CB | PRO | A | 30 | 8.980 | 36.005 | 13.078 | 1.00 | 53.75 | C |
| ATOM | 231 | CG | PRO | A | 30 | 8.779 | 35.303 | 14.402 | 1.00 | 53.15 | C |
| ATOM | 232 | CD | PRO | A | 30 | 9.052 | 33.848 | 14.115 | 1.00 | 54.10 | C |
| ATOM | 233 | C | PRO | A | 30 | 9.560 | 35.079 | 10.847 | 1.00 | 52.79 | C |
| ATOM | 234 | O | PRO | A | 30 | 9.272 | 35.824 | 9.930 | 1.00 | 49.86 | O |
| ATOM | 235 | N | LEU | A | 31 | 10.700 | 34.379 | 10.917 | 1.00 | 54.07 | N |
| ATOM | 236 | CA | LEU | A | 31 | 11.828 | 34.514 | 9.948 | 1.00 | 56.21 | C |
| ATOM | 237 | CB | LEU | A | 31 | 13.153 | 34.211 | 10.650 | 1.00 | 59.82 | C |
| ATOM | 238 | CG | LEU | A | 31 | 13.686 | 35.292 | 11.590 | 1.00 | 65.83 | C |
| ATOM | 239 | CD1 | LEU | A | 31 | 12.721 | 35.578 | 12.727 | 1.00 | 68.75 | C |
| ATOM | 240 | CD2 | LEU | A | 31 | 15.037 | 34.880 | 12.154 | 1.00 | 64.78 | C |
| ATOM | 241 | C | LEU | A | 31 | 11.630 | 33.564 | 8.760 | 1.00 | 55.84 | C |
| ATOM | 242 | O | LEU | A | 31 | 12.395 | 33.690 | 7.780 | 1.00 | 49.10 | O |
| ATOM | 243 | N | PHE | A | 32 | 10.662 | 32.641 | 8.849 | 1.00 | 56.98 | N |
| ATOM | 244 | CA | PHE | A | 32 | 10.254 | 31.724 | 7.749 | 1.00 | 53.42 | C |
| ATOM | 245 | CB | PHE | A | 32 | 11.061 | 30.426 | 7.793 | 1.00 | 46.63 | C |
| ATOM | 246 | CG | PHE | A | 32 | 11.175 | 29.710 | 6.472 | 1.00 | 39.02 | C |
| ATOM | 247 | CD1 | PHE | A | 32 | 11.780 | 30.320 | 5.384 | 1.00 | 36.30 | C |
| ATOM | 248 | CE1 | PHE | A | 32 | 11.887 | 29.657 | 4.168 | 1.00 | 37.02 | C |
| ATOM | 249 | CZ | PHE | A | 32 | 11.436 | 28.353 | 4.048 | 1.00 | 37.20 | C |
| ATOM | 250 | CD2 | PHE | A | 32 | 10.725 | 28.404 | 6.336 | 1.00 | 35.97 | C |
| ATOM | 251 | CE2 | PHE | A | 32 | 10.869 | 27.724 | 5.138 | 1.00 | 33.73 | C |
| ATOM | 252 | C | PHE | A | 32 | 8.752 | 31.467 | 7.845 | 1.00 | 59.50 | C |
| ATOM | 253 | O | PHE | A | 32 | 8.312 | 30.366 | 8.184 | 1.00 | 64.78 | O |
| ATOM | 254 | N | PRO | A | 33 | 7.917 | 32.468 | 7.483 | 1.00 | 63.99 | N |
| ATOM | 255 | CA | PRO | A | 33 | 6.472 | 32.357 | 7.664 | 1.00 | 57.07 | C |
| ATOM | 256 | CB | PRO | A | 33 | 6.037 | 33.822 | 7.496 | 1.00 | 57.58 | C |
| ATOM | 257 | CG | PRO | A | 33 | 6.964 | 34.327 | 6.417 | 1.00 | 56.55 | C |
| ATOM | 258 | CD | PRO | A | 33 | 8.300 | 33.719 | 6.794 | 1.00 | 60.44 | C |
| ATOM | 259 | C | PRO | A | 33 | 5.777 | 31.453 | 6.629 | 1.00 | 51.69 | C |
| ATOM | 260 | O | PRO | A | 33 | 6.311 | 31.178 | 5.555 | 1.00 | 45.06 | O |
| ATOM | 261 | N | ALA | A | 34 | 4.557 | 31.060 | 6.975 | 1.00 | 47.17 | N |
| ATOM | 262 | CA | ALA | A | 34 | 3.663 | 30.181 | 6.197 | 1.00 | 48.61 | C |
| ATOM | 263 | CB | ALA | A | 34 | 2.615 | 29.616 | 7.136 | 1.00 | 55.67 | C |
| ATOM | 264 | C | ALA | A | 34 | 3.028 | 30.981 | 5.060 | 1.00 | 48.26 | C |
| ATOM | 265 | O | ALA | A | 34 | 1.787 | 31.121 | 5.050 | 1.00 | 56.51 | O |
| ATOM | 266 | N | THR | A | 35 | 3.841 | 31.492 | 4.138 | 1.00 | 45.05 | N |
| ATOM | 267 | CA | THR | A | 35 | 3.392 | 32.394 | 3.049 | 1.00 | 45.26 | C |
| ATOM | 268 | CB | THR | A | 35 | 3.543 | 33.873 | 3.428 | 1.00 | 47.29 | C |
| ATOM | 269 | OG1 | THR | A | 35 | 4.892 | 34.269 | 3.178 | 1.00 | 51.15 | O |
| ATOM | 270 | CG2 | THR | A | 35 | 3.196 | 34.159 | 4.871 | 1.00 | 49.43 | C |
| ATOM | 271 | C | THR | A | 35 | 4.218 | 32.113 | 1.799 | 1.00 | 43.92 | C |
| ATOM | 272 | O | THR | A | 35 | 5.193 | 31.365 | 1.881 | 1.00 | 45.41 | O |
| ATOM | 273 | N | ASP | A | 36 | 3.878 | 32.749 | 0.694 | 1.00 | 44.66 | N |
| ATOM | 274 | CA | ASP | A | 36 | 4.534 | 32.455 | −0.597 | 1.00 | 49.23 | C |
| ATOM | 275 | CB | ASP | A | 36 | 3.702 | 32.999 | −1.760 | 1.00 | 52.44 | C |
| ATOM | 276 | CG | ASP | A | 36 | 2.483 | 32.143 | −2.093 | 1.00 | 57.60 | C |
| ATOM | 277 | OD1 | ASP | A | 36 | 2.170 | 31.219 | −1.309 | 1.00 | 48.81 | O |
| ATOM | 278 | OD2 | ASP | A | 36 | 1.862 | 32.388 | −3.153 | 1.00 | 66.51 | O |
| ATOM | 279 | C | ASP | A | 36 | 5.978 | 32.963 | −0.545 | 1.00 | 49.74 | C |
| ATOM | 280 | O | ASP | A | 36 | 6.754 | 32.552 | −1.415 | 1.00 | 47.78 | O |
| ATOM | 281 | N | ASP | A | 37 | 6.357 | 33.792 | 0.432 | 1.00 | 48.53 | N |
| ATOM | 282 | CA | ASP | A | 37 | 7.769 | 34.267 | 0.509 | 1.00 | 53.52 | C |
| ATOM | 283 | CB | ASP | A | 37 | 7.942 | 35.420 | 1.494 | 1.00 | 59.95 | C |
| ATOM | 284 | CG | ASP | A | 37 | 7.353 | 36.693 | 0.918 | 1.00 | 65.25 | C |
| ATOM | 285 | OD1 | ASP | A | 37 | 7.457 | 36.871 | −0.335 | 1.00 | 58.81 | O |
| ATOM | 286 | OD2 | ASP | A | 37 | 6.753 | 37.454 | 1.708 | 1.00 | 65.12 | O |
| ATOM | 287 | C | ASP | A | 37 | 8.716 | 33.102 | 0.823 | 1.00 | 51.19 | C |
| ATOM | 288 | O | ASP | A | 37 | 9.847 | 33.131 | 0.275 | 1.00 | 50.50 | O |
| ATOM | 289 | N | SER | A | 38 | 8.249 | 32.130 | 1.624 | 1.00 | 42.71 | N |
| ATOM | 290 | CA | SER | A | 38 | 8.941 | 30.872 | 1.990 | 1.00 | 37.27 | C |
| ATOM | 291 | CB | SER | A | 38 | 8.331 | 30.271 | 3.198 | 1.00 | 40.35 | C |
| ATOM | 292 | OG | SER | A | 38 | 8.308 | 31.186 | 4.283 | 1.00 | 39.37 | O |
| ATOM | 293 | C | SER | A | 38 | 8.938 | 29.847 | 0.851 | 1.00 | 38.36 | C |
| ATOM | 294 | O | SER | A | 38 | 9.681 | 28.864 | 0.996 | 1.00 | 39.18 | O |
| ATOM | 295 | N | LEU | A | 39 | 8.203 | 30.047 | −0.254 | 1.00 | 38.07 | N |
| ATOM | 296 | CA | LEU | A | 39 | 8.084 | 29.013 | −1.322 | 1.00 | 37.93 | C |
| ATOM | 297 | CB | LEU | A | 39 | 6.613 | 28.705 | −1.626 | 1.00 | 41.21 | C |
| ATOM | 298 | CG | LEU | A | 39 | 5.738 | 28.220 | −0.473 | 1.00 | 45.86 | C |
| ATOM | 299 | CD1 | LEU | A | 39 | 4.317 | 27.992 | −0.983 | 1.00 | 48.56 | C |
| ATOM | 300 | CD2 | LEU | A | 39 | 6.286 | 26.949 | 0.176 | 1.00 | 45.78 | C |
| ATOM | 301 | C | LEU | A | 39 | 8.752 | 29.455 | −2.624 | 1.00 | 38.21 | C |
| ATOM | 302 | O | LEU | A | 39 | 9.284 | 28.583 | −3.351 | 1.00 | 39.99 | O |
| ATOM | 303 | N | TYR | A | 40 | 8.599 | 30.710 | −3.020 | 1.00 | 40.20 | N |
| ATOM | 304 | CA | TYR | A | 40 | 8.921 | 31.122 | −4.410 | 1.00 | 43.25 | C |
| ATOM | 305 | CB | TYR | A | 40 | 7.654 | 31.377 | −5.226 | 1.00 | 47.63 | C |
| ATOM | 306 | CG | TYR | A | 40 | 6.587 | 30.316 | −5.094 | 1.00 | 50.40 | C |
| ATOM | 307 | CD1 | TYR | A | 40 | 6.820 | 29.003 | −5.475 | 1.00 | 50.68 | C |
| ATOM | 308 | CE1 | TYR | A | 40 | 5.841 | 28.028 | −5.367 | 1.00 | 48.52 | C |
| ATOM | 309 | CZ | TYR | A | 40 | 4.601 | 28.357 | −4.849 | 1.00 | 54.45 | C |
| ATOM | 310 | OH | TYR | A | 40 | 3.613 | 27.419 | −4.704 | 1.00 | 52.42 | O |
| ATOM | 311 | CE2 | TYR | A | 40 | 4.355 | 29.661 | −4.457 | 1.00 | 52.46 | C |
| ATOM | 312 | CD2 | TYR | A | 40 | 5.343 | 30.623 | −4.581 | 1.00 | 48.78 | C |
| ATOM | 313 | C | TYR | A | 40 | 9.840 | 32.336 | −4.392 | 1.00 | 43.11 | C |
| ATOM | 314 | O | TYR | A | 40 | 9.838 | 33.109 | −3.433 | 1.00 | 43.23 | O |
| ATOM | 315 | N | TYR | A | 41 | 10.649 | 32.446 | −5.434 | 1.00 | 46.67 | N |
| ATOM | 316 | CA | TYR | A | 41 | 11.429 | 33.663 | −5.764 | 1.00 | 52.93 | C |
| ATOM | 317 | CB | TYR | A | 41 | 12.288 | 33.363 | −7.001 | 1.00 | 55.31 | C |
| ATOM | 318 | CG | TYR | A | 41 | 13.468 | 32.433 | −6.802 | 1.00 | 50.05 | C |
| ATOM | 319 | CD1 | TYR | A | 41 | 14.249 | 32.469 | −5.655 | 1.00 | 51.19 | C |
| ATOM | 320 | CE1 | TYR | A | 41 | 15.338 | 31.629 | −5.490 | 1.00 | 51.50 | C |
| ATOM | 321 | CZ | TYR | A | 41 | 15.689 | 30.748 | −6.495 | 1.00 | 51.53 | C |
| ATOM | 322 | OH | TYR | A | 41 | 16.782 | 29.945 | −6.353 | 1.00 | 56.87 | O |
| ATOM | 323 | CE2 | TYR | A | 41 | 14.920 | 30.685 | −7.641 | 1.00 | 53.45 | C |
| ATOM | 324 | CD2 | TYR | A | 41 | 13.830 | 31.531 | −7.789 | 1.00 | 51.50 | C |
| ATOM | 325 | C | TYR | A | 41 | 10.450 | 34.860 | −5.872 | 1.00 | 54.48 | C |
| ATOM | 326 | O | TYR | A | 41 | 9.229 | 34.619 | −6.044 | 1.00 | 49.45 | O |
| ATOM | 327 | N | LYS | A | 42 | 10.961 | 36.098 | −5.734 | 1.00 | 57.52 | N |
| ATOM | 328 | CA | LYS | A | 42 | 10.203 | 37.345 | −5.390 | 1.00 | 61.33 | C |
| ATOM | 329 | CB | LYS | A | 42 | 11.120 | 38.570 | −5.507 | 1.00 | 61.98 | C |
| ATOM | 330 | C | LYS | A | 42 | 8.941 | 37.532 | −6.261 | 1.00 | 61.23 | C |
| ATOM | 331 | O | LYS | A | 42 | 7.849 | 37.789 | −5.687 | 1.00 | 59.01 | O |
| ATOM | 332 | N | GLY | A | 43 | 9.053 | 37.470 | −7.590 | 1.00 | 58.31 | N |
| ATOM | 333 | CA | GLY | A | 43 | 7.901 | 37.767 | −8.469 | 1.00 | 67.06 | C |
| ATOM | 334 | C | GLY | A | 43 | 7.196 | 36.518 | −8.976 | 1.00 | 68.16 | C |
| ATOM | 335 | O | GLY | A | 43 | 6.227 | 36.670 | −9.737 | 1.00 | 66.29 | O |
| ATOM | 336 | N | THR | A | 44 | 7.680 | 35.327 | −8.612 | 1.00 | 74.49 | N |
| ATOM | 337 | CA | THR | A | 44 | 7.309 | 34.021 | −9.235 | 1.00 | 77.76 | C |
| ATOM | 338 | CB | THR | A | 44 | 8.240 | 32.913 | −8.714 | 1.00 | 79.99 | C |
| ATOM | 339 | OG1 | THR | A | 44 | 9.586 | 33.337 | −8.949 | 1.00 | 75.75 | O |
| ATOM | 340 | CG2 | THR | A | 44 | 7.995 | 31.560 | −9.349 | 1.00 | 81.15 | C |
| ATOM | 341 | C | THR | A | 44 | 5.823 | 33.738 | −8.987 | 1.00 | 69.84 | C |
| ATOM | 342 | O | THR | A | 44 | 5.363 | 33.857 | −7.853 | 1.00 | 55.30 | O |
| ATOM | 343 | N | PRO | A | 45 | 5.039 | 33.340 | −10.022 | 1.00 | 73.52 | N |
| ATOM | 344 | CA | PRO | A | 45 | 3.594 | 33.123 | −9.881 | 1.00 | 74.09 | C |
| ATOM | 345 | CB | PRO | A | 45 | 3.127 | 32.684 | −11.279 | 1.00 | 72.72 | C |
| ATOM | 346 | CG | PRO | A | 45 | 4.391 | 32.224 | −11.982 | 1.00 | 77.74 | C |
| ATOM | 347 | CD | PRO | A | 45 | 5.500 | 33.072 | −11.395 | 1.00 | 82.74 | C |
| ATOM | 348 | C | PRO | A | 45 | 3.224 | 32.080 | −8.812 | 1.00 | 78.53 | C |
| ATOM | 349 | O | PRO | A | 45 | 2.570 | 32.471 | −7.849 | 1.00 | 86.68 | O |
| ATOM | 350 | N | GLY | A | 46 | 3.593 | 30.808 | −9.022 | 1.00 | 70.29 | N |
| ATOM | 351 | CA | GLY | A | 46 | 3.634 | 29.750 | −7.991 | 1.00 | 64.04 | C |
| ATOM | 352 | C | GLY | A | 46 | 2.263 | 29.176 | −7.632 | 1.00 | 58.05 | C |
| ATOM | 353 | O | GLY | A | 46 | 1.340 | 29.930 | −7.334 | 1.00 | 61.20 | O |
| ATOM | 354 | N | PRO | A | 47 | 2.099 | 27.829 | −7.558 | 1.00 | 52.56 | N |
| ATOM | 355 | CA | PRO | A | 47 | 0.827 | 27.229 | −7.155 | 1.00 | 53.25 | C |
| ATOM | 356 | CB | PRO | A | 47 | 1.126 | 25.727 | −7.006 | 1.00 | 51.27 | C |
| ATOM | 357 | CG | PRO | A | 47 | 2.346 | 25.499 | −7.868 | 1.00 | 48.88 | C |
| ATOM | 358 | CD | PRO | A | 47 | 3.111 | 26.804 | −7.853 | 1.00 | 47.83 | C |
| ATOM | 359 | C | PRO | A | 47 | 0.335 | 27.755 | −5.801 | 1.00 | 51.10 | C |
| ATOM | 360 | O | PRO | A | 47 | 1.139 | 28.025 | −4.945 | 1.00 | 54.50 | O |
| ATOM | 361 | N | ALA | A | 48 | −0.976 | 27.839 | −5.632 | 1.00 | 42.60 | N |
| ATOM | 362 | CA | ALA | A | 48 | −1.609 | 28.165 | −4.340 | 1.00 | 41.34 | C |
| ATOM | 363 | CB | ALA | A | 48 | −3.044 | 28.582 | −4.533 | 1.00 | 37.79 | C |
| ATOM | 364 | C | ALA | A | 48 | −1.519 | 26.941 | −3.435 | 1.00 | 38.56 | C |
| ATOM | 365 | O | ALA | A | 48 | −1.732 | 25.816 | −3.913 | 1.00 | 44.54 | O |
| ATOM | 366 | N | VAL | A | 49 | −1.257 | 27.151 | −2.158 | 1.00 | 33.35 | N |
| ATOM | 367 | CA | VAL | A | 49 | −1.173 | 26.021 | −1.206 | 1.00 | 36.32 | C |
| ATOM | 368 | CB | VAL | A | 49 | 0.296 | 25.646 | −0.884 | 1.00 | 36.30 | C |
| ATOM | 369 | CG1 | VAL | A | 49 | 1.131 | 25.510 | −2.151 | 1.00 | 33.67 | C |
| ATOM | 370 | CG2 | VAL | A | 49 | 0.934 | 26.639 | 0.073 | 1.00 | 35.08 | C |
| ATOM | 371 | C | VAL | A | 49 | −1.977 | 26.456 | 0.010 | 1.00 | 35.80 | C |
| ATOM | 372 | O | VAL | A | 49 | −2.314 | 27.629 | 0.058 | 1.00 | 38.41 | O |
| ATOM | 373 | N | ARG | A | 50 | −2.332 | 25.517 | 0.887 | 1.00 | 36.97 | N |
| ATOM | 374 | CA | ARG | A | 50 | −2.775 | 25.806 | 2.266 | 1.00 | 37.86 | C |
| ATOM | 375 | CB | ARG | A | 50 | −4.140 | 25.185 | 2.600 | 1.00 | 40.94 | C |
| ATOM | 376 | CG | ARG | A | 50 | −5.245 | 25.407 | 1.577 | 1.00 | 42.37 | C |
| ATOM | 377 | CD | ARG | A | 50 | −6.627 | 24.907 | 2.016 | 1.00 | 44.38 | C |
| ATOM | 378 | NE | ARG | A | 50 | −7.577 | 25.318 | 0.999 | 1.00 | 47.12 | N |
| ATOM | 379 | CZ | ARG | A | 50 | −8.491 | 24.567 | 0.402 | 1.00 | 48.13 | C |
| ATOM | 380 | NH1 | ARG | A | 50 | −8.701 | 23.306 | 0.747 | 1.00 | 52.10 | N |
| ATOM | 381 | NH2 | ARG | A | 50 | −9.217 | 25.109 | −0.552 | 1.00 | 45.07 | N |
| ATOM | 382 | C | ARG | A | 50 | −1.691 | 25.240 | 3.185 | 1.00 | 41.24 | C |
| ATOM | 383 | O | ARG | A | 50 | −1.221 | 24.105 | 2.951 | 1.00 | 41.44 | O |
| ATOM | 384 | N | TRP | A | 51 | −1.286 | 26.013 | 4.177 | 1.00 | 42.01 | N |
| ATOM | 385 | CA | TRP | A | 51 | −0.228 | 25.602 | 5.118 | 1.00 | 44.93 | C |
| ATOM | 386 | CB | TRP | A | 51 | 0.596 | 26.812 | 5.580 | 1.00 | 47.75 | C |
| ATOM | 387 | CG | TRP | A | 51 | 1.513 | 27.387 | 4.542 | 1.00 | 42.64 | C |
| ATOM | 388 | CD1 | TRP | A | 51 | 1.181 | 28.233 | 3.521 | 1.00 | 42.35 | C |
| ATOM | 389 | NE1 | TRP | A | 51 | 2.295 | 28.566 | 2.798 | 1.00 | 41.73 | N |
| ATOM | 390 | CE2 | TRP | A | 51 | 3.384 | 27.933 | 3.338 | 1.00 | 40.61 | C |
| ATOM | 391 | CD2 | TRP | A | 51 | 2.936 | 27.191 | 4.452 | 1.00 | 41.96 | C |
| ATOM | 392 | CE3 | TRP | A | 51 | 3.871 | 26.455 | 5.193 | 1.00 | 41.61 | C |
| ATOM | 393 | CZ3 | TRP | A | 51 | 5.198 | 26.496 | 4.826 | 1.00 | 37.07 | C |
| ATOM | 394 | CH2 | TRP | A | 51 | 5.616 | 27.248 | 3.732 | 1.00 | 39.11 | C |
| ATOM | 395 | CZ2 | TRP | A | 51 | 4.726 | 27.964 | 2.964 | 1.00 | 39.45 | C |
| ATOM | 396 | C | TRP | A | 51 | −0.955 | 24.906 | 6.256 | 1.00 | 46.61 | C |
| ATOM | 397 | O | TRP | A | 51 | −1.838 | 25.560 | 6.808 | 1.00 | 46.10 | O |
| ATOM | 398 | N | LYS | A | 52 | −0.623 | 23.636 | 6.530 | 1.00 | 45.39 | N |
| ATOM | 399 | CA | LYS | A | 52 | −1.270 | 22.806 | 7.573 | 1.00 | 42.47 | C |
| ATOM | 400 | CB | LYS | A | 52 | −2.101 | 21.696 | 6.930 | 1.00 | 41.12 | C |
| ATOM | 401 | CG | LYS | A | 52 | −3.442 | 22.179 | 6.399 | 1.00 | 45.47 | C |
| ATOM | 402 | CD | LYS | A | 52 | −4.271 | 21.166 | 5.642 | 1.00 | 49.31 | C |
| ATOM | 403 | CE | LYS | A | 52 | −5.390 | 20.579 | 6.477 | 1.00 | 57.61 | C |
| ATOM | 404 | NZ | LYS | A | 52 | −6.459 | 21.563 | 6.775 | 1.00 | 70.93 | N |
| ATOM | 405 | C | LYS | A | 52 | −0.205 | 22.241 | 8.506 | 1.00 | 48.12 | C |
| ATOM | 406 | O | LYS | A | 52 | 0.872 | 21.833 | 8.028 | 1.00 | 46.80 | O |
| ATOM | 407 | N | ARG | A | 53 | −0.530 | 22.217 | 9.796 | 1.00 | 51.36 | N |
| ATOM | 408 | CA | ARG | A | 53 | 0.213 | 21.477 | 10.844 | 1.00 | 46.49 | C |
| ATOM | 409 | CB | ARG | A | 53 | −0.252 | 21.977 | 12.214 | 1.00 | 48.62 | C |
| ATOM | 410 | CG | ARG | A | 53 | −0.015 | 23.467 | 12.390 | 1.00 | 49.30 | C |
| ATOM | 411 | CD | ARG | A | 53 | 0.245 | 23.884 | 13.811 | 1.00 | 54.41 | C |
| ATOM | 412 | NE | ARG | A | 53 | 0.558 | 25.304 | 13.846 | 1.00 | 56.64 | N |
| ATOM | 413 | CZ | ARG | A | 53 | 1.773 | 25.818 | 13.942 | 1.00 | 58.39 | C |
| ATOM | 414 | NH1 | ARG | A | 53 | 2.840 | 25.037 | 14.023 | 1.00 | 59.68 | N |
| ATOM | 415 | NH2 | ARG | A | 53 | 1.911 | 27.131 | 13.974 | 1.00 | 59.57 | N |
| ATOM | 416 | C | ARG | A | 53 | −0.025 | 19.985 | 10.640 | 1.00 | 43.49 | C |
| ATOM | 417 | O | ARG | A | 53 | −1.082 | 19.578 | 10.148 | 1.00 | 37.47 | O |
| ATOM | 418 | N | PRO | A | 54 | 0.958 | 19.125 | 10.987 | 1.00 | 43.67 | N |
| ATOM | 419 | CA | PRO | A | 54 | 0.843 | 17.686 | 10.741 | 1.00 | 45.04 | C |
| ATOM | 420 | CB | PRO | A | 54 | 2.076 | 17.105 | 11.467 | 1.00 | 41.18 | C |
| ATOM | 421 | CG | PRO | A | 54 | 3.071 | 18.217 | 11.411 | 1.00 | 40.17 | C |
| ATOM | 422 | CD | PRO | A | 54 | 2.241 | 19.473 | 11.608 | 1.00 | 43.37 | C |
| ATOM | 423 | C | PRO | A | 54 | −0.497 | 17.087 | 11.221 | 1.00 | 43.44 | C |
| ATOM | 424 | O | PRO | A | 54 | −1.021 | 16.299 | 10.468 | 1.00 | 43.07 | O |
| ATOM | 425 | N | LYS | A | 55 | −1.016 | 17.470 | 12.404 | 1.00 | 45.10 | N |
| ATOM | 426 | CA | LYS | A | 55 | −2.343 | 17.017 | 12.937 | 1.00 | 50.05 | C |
| ATOM | 427 | CB | LYS | A | 55 | −2.718 | 17.656 | 14.285 | 1.00 | 47.53 | C |
| ATOM | 428 | C | LYS | A | 55 | −3.428 | 17.320 | 11.896 | 1.00 | 49.10 | C |
| ATOM | 429 | O | LYS | A | 55 | −4.295 | 16.490 | 11.697 | 1.00 | 54.47 | O |
| ATOM | 430 | N | GLY | A | 56 | −3.347 | 18.466 | 11.233 | 1.00 | 47.98 | N |
| ATOM | 431 | CA | GLY | A | 56 | −4.352 | 18.902 | 10.258 | 1.00 | 47.72 | C |
| ATOM | 432 | C | GLY | A | 56 | −4.302 | 18.053 | 9.014 | 1.00 | 48.35 | C |
| ATOM | 433 | O | GLY | A | 56 | −5.247 | 18.137 | 8.203 | 1.00 | 54.36 | O |
| ATOM | 434 | N | ILE | A | 57 | −3.228 | 17.289 | 8.835 | 1.00 | 51.97 | N |
| ATOM | 435 | CA | ILE | A | 57 | −3.024 | 16.437 | 7.626 | 1.00 | 48.60 | C |
| ATOM | 436 | CB | ILE | A | 57 | −1.569 | 16.525 | 7.141 | 1.00 | 50.40 | C |
| ATOM | 437 | CG1 | ILE | A | 57 | −1.229 | 17.959 | 6.712 | 1.00 | 50.61 | C |
| ATOM | 438 | CG2 | ILE | A | 57 | −1.313 | 15.497 | 6.041 | 1.00 | 46.98 | C |
| ATOM | 439 | CD1 | ILE | A | 57 | 0.247 | 18.244 | 6.592 | 1.00 | 49.25 | C |
| ATOM | 440 | C | ILE | A | 57 | −3.430 | 15.000 | 7.951 | 1.00 | 47.56 | C |
| ATOM | 441 | O | ILE | A | 57 | −3.947 | 14.318 | 7.058 | 1.00 | 49.74 | O |
| ATOM | 442 | N | CYS | A | 58 | −3.216 | 14.548 | 9.185 | 1.00 | 53.65 | N |
| ATOM | 443 | CA | CYS | A | 58 | −3.478 | 13.143 | 9.582 | 1.00 | 52.40 | C |
| ATOM | 444 | CB | CYS | A | 58 | −2.426 | 12.226 | 8.994 | 1.00 | 53.88 | C |
| ATOM | 445 | SG | CYS | A | 58 | −3.047 | 10.533 | 8.900 | 1.00 | 62.02 | S |
| ATOM | 446 | C | CYS | A | 58 | −3.490 | 12.979 | 11.100 | 1.00 | 53.03 | C |
| ATOM | 447 | O | CYS | A | 58 | −2.747 | 13.678 | 11.752 | 1.00 | 65.17 | O |
| ATOM | 448 | N | GLU | A | 59 | −4.224 | 11.996 | 11.608 | 1.00 | 66.85 | N |
| ATOM | 449 | CA | GLU | A | 59 | −4.737 | 11.986 | 13.003 | 1.00 | 71.64 | C |
| ATOM | 450 | CB | GLU | A | 59 | −5.908 | 11.007 | 13.152 | 1.00 | 78.67 | C |
| ATOM | 451 | CG | GLU | A | 59 | −6.868 | 11.365 | 14.279 | 1.00 | 85.99 | C |
| ATOM | 452 | CD | GLU | A | 59 | −7.465 | 12.763 | 14.184 | 1.00 | 93.88 | C |
| ATOM | 453 | OE1 | GLU | A | 59 | −8.236 | 13.019 | 13.232 | 1.00 | 100.38 | O |
| ATOM | 454 | OE2 | GLU | A | 59 | −7.142 | 13.603 | 15.051 | 1.00 | 87.86 | O |
| ATOM | 455 | C | GLU | A | 59 | −3.614 | 11.681 | 14.002 | 1.00 | 66.16 | C |
| ATOM | 456 | O | GLU | A | 59 | −3.737 | 12.128 | 15.163 | 1.00 | 81.24 | O |
| ATOM | 457 | N | ASP | A | 60 | −2.549 | 10.985 | 13.623 | 1.00 | 54.47 | N |
| ATOM | 458 | CA | ASP | A | 60 | −1.570 | 10.550 | 14.660 | 1.00 | 55.90 | C |
| ATOM | 459 | CB | ASP | A | 60 | −1.708 | 9.050 | 14.934 | 1.00 | 56.53 | C |
| ATOM | 460 | CG | ASP | A | 60 | −0.894 | 8.581 | 16.129 | 1.00 | 65.77 | C |
| ATOM | 461 | OD1 | ASP | A | 60 | −0.517 | 9.440 | 16.940 | 1.00 | 73.89 | O |
| ATOM | 462 | OD2 | ASP | A | 60 | −0.615 | 7.366 | 16.230 | 1.00 | 69.20 | O |
| ATOM | 463 | C | ASP | A | 60 | −0.149 | 10.953 | 14.260 | 1.00 | 52.39 | C |
| ATOM | 464 | O | ASP | A | 60 | 0.716 | 10.104 | 14.052 | 1.00 | 46.44 | O |
| ATOM | 465 | N | PRO | A | 61 | 0.167 | 12.262 | 14.188 | 1.00 | 47.22 | N |
| ATOM | 466 | CA | PRO | A | 61 | 1.413 | 12.706 | 13.568 | 1.00 | 53.17 | C |
| ATOM | 467 | CB | PRO | A | 61 | 1.351 | 14.240 | 13.637 | 1.00 | 49.63 | C |
| ATOM | 468 | CG | PRO | A | 61 | 0.397 | 14.509 | 14.773 | 1.00 | 47.90 | C |
| ATOM | 469 | CD | PRO | A | 61 | −0.615 | 13.382 | 14.716 | 1.00 | 47.15 | C |
| ATOM | 470 | C | PRO | A | 61 | 2.629 | 12.136 | 14.308 | 1.00 | 50.63 | C |
| ATOM | 471 | O | PRO | A | 61 | 2.630 | 12.131 | 15.512 | 1.00 | 49.06 | O |
| ATOM | 472 | N | ARG | A | 62 | 3.605 | 11.641 | 13.544 | 1.00 | 50.34 | N |
| ATOM | 473 | CA | ARG | A | 62 | 4.877 | 11.082 | 14.061 | 1.00 | 46.60 | C |
| ATOM | 474 | CB | ARG | A | 62 | 4.825 | 9.552 | 14.047 | 1.00 | 53.99 | C |
| ATOM | 475 | CG | ARG | A | 62 | 3.968 | 8.933 | 15.139 | 1.00 | 57.30 | C |
| ATOM | 476 | CD | ARG | A | 62 | 4.558 | 9.144 | 16.526 | 1.00 | 65.23 | C |
| ATOM | 477 | NE | ARG | A | 62 | 3.920 | 8.277 | 17.505 | 1.00 | 66.63 | N |
| ATOM | 478 | CZ | ARG | A | 62 | 2.663 | 8.403 | 17.915 | 1.00 | 66.51 | C |
| ATOM | 479 | NH1 | ARG | A | 62 | 1.900 | 9.381 | 17.454 | 1.00 | 67.90 | N |
| ATOM | 480 | NH2 | ARG | A | 62 | 2.172 | 7.552 | 18.797 | 1.00 | 64.24 | N |
| ATOM | 481 | C | ARG | A | 62 | 6.021 | 11.580 | 13.182 | 1.00 | 42.46 | C |
| ATOM | 482 | O | ARG | A | 62 | 5.802 | 11.710 | 11.960 | 1.00 | 49.07 | O |
| ATOM | 483 | N | LEU | A | 63 | 7.185 | 11.851 | 13.783 | 1.00 | 44.24 | N |
| ATOM | 484 | CA | LEU | A | 63 | 8.480 | 12.036 | 13.065 | 1.00 | 45.45 | C |
| ATOM | 485 | CB | LEU | A | 63 | 9.611 | 12.333 | 14.052 | 1.00 | 48.44 | C |
| ATOM | 486 | CG | LEU | A | 63 | 9.930 | 13.792 | 14.338 | 1.00 | 50.00 | C |
| ATOM | 487 | CD1 | LEU | A | 63 | 11.221 | 13.881 | 15.134 | 1.00 | 52.58 | C |
| ATOM | 488 | CD2 | LEU | A | 63 | 10.056 | 14.596 | 13.062 | 1.00 | 50.01 | C |
| ATOM | 489 | C | LEU | A | 63 | 8.816 | 10.746 | 12.317 | 1.00 | 42.38 | C |
| ATOM | 490 | O | LEU | A | 63 | 9.153 | 10.814 | 11.126 | 1.00 | 37.75 | O |
| ATOM | 491 | N | PHE | A | 64 | 8.761 | 9.625 | 13.029 | 1.00 | 38.14 | N |
| ATOM | 492 | CA | PHE | A | 64 | 9.062 | 8.280 | 12.490 | 1.00 | 41.36 | C |
| ATOM | 493 | CB | PHE | A | 64 | 10.369 | 7.732 | 13.072 | 1.00 | 42.35 | C |
| ATOM | 494 | CG | PHE | A | 64 | 11.532 | 8.691 | 13.054 | 1.00 | 44.00 | C |
| ATOM | 495 | CD1 | PHE | A | 64 | 12.177 | 9.013 | 11.870 | 1.00 | 48.44 | C |
| ATOM | 496 | CE1 | PHE | A | 64 | 13.256 | 9.883 | 11.858 | 1.00 | 47.08 | C |
| ATOM | 497 | CZ | PHE | A | 64 | 13.724 | 10.425 | 13.032 | 1.00 | 46.10 | C |
| ATOM | 498 | CD2 | PHE | A | 64 | 12.013 | 9.248 | 14.226 | 1.00 | 46.60 | C |
| ATOM | 499 | CE2 | PHE | A | 64 | 13.105 | 10.107 | 14.216 | 1.00 | 47.70 | C |
| ATOM | 500 | C | PHE | A | 64 | 7.869 | 7.378 | 12.806 | 1.00 | 40.29 | C |
| ATOM | 501 | O | PHE | A | 64 | 7.392 | 7.352 | 13.963 | 1.00 | 41.55 | O |
| ATOM | 502 | N | VAL | A | 65 | 7.367 | 6.713 | 11.776 | 1.00 | 39.72 | N |
| ATOM | 503 | CA | VAL | A | 65 | 6.325 | 5.667 | 11.905 | 1.00 | 46.83 | C |
| ATOM | 504 | CB | VAL | A | 65 | 5.233 | 5.844 | 10.831 | 1.00 | 46.48 | C |
| ATOM | 505 | CG1 | VAL | A | 65 | 4.285 | 4.653 | 10.768 | 1.00 | 45.19 | C |
| ATOM | 506 | CG2 | VAL | A | 65 | 4.463 | 7.144 | 11.053 | 1.00 | 47.03 | C |
| ATOM | 507 | C | VAL | A | 65 | 7.096 | 4.355 | 11.795 | 1.00 | 49.63 | C |
| ATOM | 508 | O | VAL | A | 65 | 7.640 | 4.142 | 10.719 | 1.00 | 41.94 | O |
| ATOM | 509 | N | ASP | A | 66 | 7.194 | 3.585 | 12.889 | 1.00 | 58.34 | N |
| ATOM | 510 | CA | ASP | A | 66 | 8.038 | 2.360 | 13.030 | 1.00 | 72.97 | C |
| ATOM | 511 | CB | ASP | A | 66 | 7.550 | 1.183 | 12.167 | 1.00 | 75.48 | C |
| ATOM | 512 | CG | ASP | A | 66 | 6.064 | 1.133 | 11.851 | 1.00 | 75.13 | C |
| ATOM | 513 | OD1 | ASP | A | 66 | 5.244 | 1.405 | 12.775 | 1.00 | 74.19 | O |
| ATOM | 514 | OD2 | ASP | A | 66 | 5.743 | 0.818 | 10.672 | 1.00 | 65.37 | O |
| ATOM | 515 | C | ASP | A | 66 | 9.505 | 2.679 | 12.667 | 1.00 | 83.12 | C |
| ATOM | 516 | O | ASP | A | 66 | 10.142 | 1.850 | 11.952 | 1.00 | 85.80 | O |
| ATOM | 517 | N | GLY | A | 67 | 10.041 | 3.815 | 13.132 | 1.00 | 79.64 | N |
| ATOM | 518 | CA | GLY | A | 67 | 11.453 | 4.199 | 12.917 | 1.00 | 95.01 | C |
| ATOM | 519 | C | GLY | A | 67 | 11.792 | 4.507 | 11.456 | 1.00 | 106.14 | C |
| ATOM | 520 | O | GLY | A | 67 | 10.935 | 4.272 | 10.571 | 1.00 | 97.73 | O |
| ATOM | 521 | N | ILE | A | 68 | 13.031 | 4.960 | 11.208 | 1.00 | 108.35 | N |
| ATOM | 522 | CA | ILE | A | 68 | 13.470 | 5.683 | 9.967 | 1.00 | 100.26 | C |
| ATOM | 523 | CB | ILE | A | 68 | 14.575 | 6.728 | 10.263 | 1.00 | 100.08 | C |
| ATOM | 524 | CG1 | ILE | A | 68 | 15.897 | 6.459 | 9.533 | 1.00 | 98.54 | C |
| ATOM | 525 | CG2 | ILE | A | 68 | 14.797 | 6.899 | 11.761 | 1.00 | 105.79 | C |
| ATOM | 526 | CD1 | ILE | A | 68 | 16.820 | 5.469 | 10.216 | 1.00 | 99.63 | C |
| ATOM | 527 | C | ILE | A | 68 | 13.917 | 4.653 | 8.927 | 1.00 | 97.02 | C |
| ATOM | 528 | O | ILE | A | 68 | 14.211 | 3.505 | 9.329 | 1.00 | 106.47 | O |
| ATOM | 529 | N | SER | A | 69 | 13.970 | 5.051 | 7.648 | 1.00 | 89.67 | N |
| ATOM | 530 | CA | SER | A | 69 | 14.380 | 4.193 | 6.498 | 1.00 | 92.21 | C |
| ATOM | 531 | CB | SER | A | 69 | 15.838 | 3.794 | 6.584 | 1.00 | 94.59 | C |
| ATOM | 532 | OG | SER | A | 69 | 16.043 | 2.765 | 7.546 | 1.00 | 97.74 | O |
| ATOM | 533 | C | SER | A | 69 | 13.471 | 2.960 | 6.423 | 1.00 | 89.95 | C |
| ATOM | 534 | O | SER | A | 69 | 13.853 | 1.977 | 5.782 | 1.00 | 83.99 | O |
| ATOM | 535 | N | SER | A | 70 | 12.309 | 3.037 | 7.073 | 1.00 | 97.70 | N |
| ATOM | 536 | CA | SER | A | 70 | 11.234 | 2.013 | 7.124 | 1.00 | 88.75 | C |
| ATOM | 537 | CB | SER | A | 70 | 10.490 | 2.172 | 8.414 | 1.00 | 87.08 | C |
| ATOM | 538 | OG | SER | A | 70 | 10.254 | 3.559 | 8.653 | 1.00 | 75.40 | O |
| ATOM | 539 | C | SER | A | 70 | 10.297 | 2.224 | 5.930 | 1.00 | 85.96 | C |
| ATOM | 540 | O | SER | A | 70 | 9.891 | 1.226 | 5.266 | 1.00 | 61.35 | O |
| ATOM | 541 | N | HIS | A | 71 | 9.962 | 3.497 | 5.684 | 1.00 | 75.88 | N |
| ATOM | 542 | CA | HIS | A | 71 | 8.989 | 3.920 | 4.647 | 1.00 | 62.85 | C |
| ATOM | 543 | CB | HIS | A | 71 | 7.929 | 4.835 | 5.251 | 1.00 | 59.42 | C |
| ATOM | 544 | CG | HIS | A | 71 | 7.208 | 4.115 | 6.328 | 1.00 | 62.52 | C |
| ATOM | 545 | ND1 | HIS | A | 71 | 6.326 | 3.105 | 6.047 | 1.00 | 60.26 | N |
| ATOM | 546 | CE1 | HIS | A | 71 | 5.889 | 2.593 | 7.179 | 1.00 | 68.12 | C |
| ATOM | 547 | NE2 | HIS | A | 71 | 6.483 | 3.232 | 8.188 | 1.00 | 64.82 | N |
| ATOM | 548 | CD2 | HIS | A | 71 | 7.334 | 4.162 | 7.670 | 1.00 | 65.13 | C |
| ATOM | 549 | C | HIS | A | 71 | 9.721 | 4.523 | 3.458 | 1.00 | 51.81 | C |
| ATOM | 550 | O | HIS | A | 71 | 10.820 | 5.130 | 3.636 | 1.00 | 48.67 | O |
| ATOM | 551 | N | ASP | A | 72 | 9.106 | 4.302 | 2.301 | 1.00 | 40.48 | N |
| ATOM | 552 | CA | ASP | A | 72 | 9.494 | 4.879 | 0.996 | 1.00 | 38.77 | C |
| ATOM | 553 | CB | ASP | A | 72 | 8.646 | 4.277 | −0.122 | 1.00 | 39.78 | C |
| ATOM | 554 | CG | ASP | A | 72 | 8.334 | 2.803 | 0.084 | 1.00 | 39.69 | C |
| ATOM | 555 | OD1 | ASP | A | 72 | 9.057 | 2.147 | 0.862 | 1.00 | 38.01 | O |
| ATOM | 556 | OD2 | ASP | A | 72 | 7.367 | 2.332 | −0.525 | 1.00 | 35.51 | O |
| ATOM | 557 | C | ASP | A | 72 | 9.392 | 6.409 | 1.093 | 1.00 | 37.26 | C |
| ATOM | 558 | O | ASP | A | 72 | 8.660 | 6.924 | 1.960 | 1.00 | 35.10 | O |
| ATOM | 559 | N | LEU | A | 73 | 10.195 | 7.087 | 0.283 | 1.00 | 32.78 | N |
| ATOM | 560 | CA | LEU | A | 73 | 10.291 | 8.551 | 0.221 | 1.00 | 34.60 | C |
| ATOM | 561 | CB | LEU | A | 73 | 11.735 | 9.002 | 0.476 | 1.00 | 38.26 | C |
| ATOM | 562 | CG | LEU | A | 73 | 12.282 | 8.783 | 1.887 | 1.00 | 38.88 | C |
| ATOM | 563 | CD1 | LEU | A | 73 | 13.627 | 9.473 | 2.024 | 1.00 | 42.15 | C |
| ATOM | 564 | CD2 | LEU | A | 73 | 11.332 | 9.271 | 2.970 | 1.00 | 41.12 | C |
| ATOM | 565 | C | LEU | A | 73 | 9.835 | 8.971 | −1.163 | 1.00 | 33.80 | C |
| ATOM | 566 | O | LEU | A | 73 | 10.137 | 8.260 | −2.139 | 1.00 | 33.26 | O |
| ATOM | 567 | N | HIS | A | 74 | 9.141 | 10.097 | −1.231 | 1.00 | 33.91 | N |
| ATOM | 568 | CA | HIS | A | 74 | 8.642 | 10.655 | −2.504 | 1.00 | 38.71 | C |
| ATOM | 569 | CB | HIS | A | 74 | 7.189 | 10.193 | −2.767 | 1.00 | 44.28 | C |
| ATOM | 570 | CG | HIS | A | 74 | 7.073 | 8.700 | −2.836 | 1.00 | 53.57 | C |
| ATOM | 571 | ND1 | HIS | A | 74 | 7.682 | 7.945 | −3.839 | 1.00 | 62.52 | N |
| ATOM | 572 | CE1 | HIS | A | 74 | 7.456 | 6.661 | −3.634 | 1.00 | 57.82 | C |
| ATOM | 573 | NE2 | HIS | A | 74 | 6.728 | 6.539 | −2.514 | 1.00 | 56.27 | N |
| ATOM | 574 | CD2 | HIS | A | 74 | 6.482 | 7.799 | −2.012 | 1.00 | 59.76 | C |
| ATOM | 575 | C | HIS | A | 74 | 8.953 | 12.148 | −2.461 | 1.00 | 35.30 | C |
| ATOM | 576 | O | HIS | A | 74 | 8.807 | 12.787 | −1.378 | 1.00 | 32.51 | O |
| ATOM | 577 | N | GLN | A | 75 | 9.562 | 12.627 | −3.533 | 1.00 | 32.27 | N |
| ATOM | 578 | CA | GLN | A | 75 | 9.603 | 14.064 | −3.837 | 1.00 | 39.53 | C |
| ATOM | 579 | CB | GLN | A | 75 | 10.673 | 14.357 | −4.888 | 1.00 | 37.14 | C |
| ATOM | 580 | CG | GLN | A | 75 | 10.187 | 14.175 | −6.323 | 1.00 | 34.68 | C |
| ATOM | 581 | CD | GLN | A | 75 | 10.047 | 12.749 | −6.790 | 1.00 | 32.27 | C |
| ATOM | 582 | OE1 | GLN | A | 75 | 10.057 | 11.808 | −5.998 | 1.00 | 31.67 | O |
| ATOM | 583 | NE2 | GLN | A | 75 | 9.928 | 12.584 | −8.103 | 1.00 | 30.75 | N |
| ATOM | 584 | C | GLN | A | 75 | 8.176 | 14.278 | −4.307 | 1.00 | 46.20 | C |
| ATOM | 585 | O | GLN | A | 75 | 7.644 | 13.352 | −4.961 | 1.00 | 70.24 | O |
| ATOM | 586 | N | GLY | A | 76 | 7.501 | 15.329 | −3.917 | 1.00 | 45.16 | N |
| ATOM | 587 | CA | GLY | A | 76 | 6.070 | 15.378 | −4.279 | 1.00 | 47.80 | C |
| ATOM | 588 | C | GLY | A | 76 | 5.884 | 15.736 | −5.746 | 1.00 | 46.18 | C |
| ATOM | 589 | O | GLY | A | 76 | 6.777 | 15.445 | −6.573 | 1.00 | 45.23 | O |
| ATOM | 590 | N | GLN | A | 77 | 4.795 | 16.432 | −6.052 | 1.00 | 53.10 | N |
| ATOM | 591 | CA | GLN | A | 77 | 4.527 | 16.992 | −7.400 | 1.00 | 52.78 | C |
| ATOM | 592 | CB | GLN | A | 77 | 3.058 | 17.425 | −7.489 | 1.00 | 62.25 | C |
| ATOM | 593 | CG | GLN | A | 77 | 2.120 | 16.300 | −7.937 | 1.00 | 72.19 | C |
| ATOM | 594 | CD | GLN | A | 77 | 2.383 | 15.832 | −9.359 | 1.00 | 79.26 | C |
| ATOM | 595 | OE1 | GLN | A | 77 | 2.182 | 16.570 | −10.325 | 1.00 | 81.05 | O |
| ATOM | 596 | NE2 | GLN | A | 77 | 2.844 | 14.596 | −9.511 | 1.00 | 75.16 | N |
| ATOM | 597 | C | GLN | A | 77 | 5.557 | 18.091 | −7.718 | 1.00 | 47.05 | C |
| ATOM | 598 | O | GLN | A | 77 | 5.856 | 18.277 | −8.910 | 1.00 | 46.65 | O |
| ATOM | 599 | N | VAL | A | 78 | 6.136 | 18.766 | −6.721 | 1.00 | 43.94 | N |
| ATOM | 600 | CA | VAL | A | 78 | 7.066 | 19.918 | −6.981 | 1.00 | 43.78 | C |
| ATOM | 601 | CB | VAL | A | 78 | 6.354 | 21.257 | −6.685 | 1.00 | 37.98 | C |
| ATOM | 602 | CG1 | VAL | A | 78 | 5.182 | 21.489 | −7.611 | 1.00 | 36.55 | C |
| ATOM | 603 | CG2 | VAL | A | 78 | 5.879 | 21.350 | −5.246 | 1.00 | 36.86 | C |
| ATOM | 604 | C | VAL | A | 78 | 8.390 | 19.779 | −6.195 | 1.00 | 43.46 | C |
| ATOM | 605 | O | VAL | A | 78 | 9.199 | 20.719 | −6.248 | 1.00 | 43.52 | O |
| ATOM | 606 | N | GLY | A | 79 | 8.616 | 18.668 | −5.486 | 1.00 | 42.02 | N |
| ATOM | 607 | CA | GLY | A | 79 | 9.840 | 18.442 | −4.693 | 1.00 | 42.91 | C |
| ATOM | 608 | C | GLY | A | 79 | 10.989 | 17.969 | −5.571 | 1.00 | 40.99 | C |
| ATOM | 609 | O | GLY | A | 79 | 10.783 | 17.789 | −6.777 | 1.00 | 43.07 | O |
| ATOM | 610 | N | ASN | A | 80 | 12.162 | 17.758 | −4.981 | 1.00 | 42.23 | N |
| ATOM | 611 | CA | ASN | A | 80 | 13.428 | 17.444 | −5.702 | 1.00 | 36.94 | C |
| ATOM | 612 | CB | ASN | A | 80 | 14.499 | 18.449 | −5.325 | 1.00 | 35.48 | C |
| ATOM | 613 | CG | ASN | A | 80 | 14.256 | 19.792 | −5.970 | 1.00 | 31.25 | C |
| ATOM | 614 | OD1 | ASN | A | 80 | 14.543 | 19.958 | −7.149 | 1.00 | 27.86 | O |
| ATOM | 615 | ND2 | ASN | A | 80 | 13.760 | 20.737 | −5.193 | 1.00 | 31.24 | N |
| ATOM | 616 | C | ASN | A | 80 | 13.936 | 16.039 | −5.374 | 1.00 | 34.95 | C |
| ATOM | 617 | O | ASN | A | 80 | 14.110 | 15.748 | −4.183 | 1.00 | 33.52 | O |
| ATOM | 618 | N | CYS | A | 81 | 14.179 | 15.218 | −6.402 | 1.00 | 37.67 | N |
| ATOM | 619 | CA | CYS | A | 81 | 14.686 | 13.820 | −6.296 | 1.00 | 37.77 | C |
| ATOM | 620 | CB | CYS | A | 81 | 14.744 | 13.146 | −7.663 | 1.00 | 37.00 | C |
| ATOM | 621 | SG | CYS | A | 81 | 13.084 | 12.817 | −8.343 | 1.00 | 43.00 | S |
| ATOM | 622 | C | CYS | A | 81 | 16.046 | 13.835 | −5.589 | 1.00 | 38.20 | C |
| ATOM | 623 | O | CYS | A | 81 | 16.307 | 12.937 | −4.765 | 1.00 | 37.67 | O |
| ATOM | 624 | N | TRP | A | 82 | 16.861 | 14.857 | −5.847 | 1.00 | 39.39 | N |
| ATOM | 625 | CA | TRP | A | 82 | 18.232 | 14.946 | −5.288 | 1.00 | 37.40 | C |
| ATOM | 626 | CB | TRP | A | 82 | 19.047 | 16.121 | −5.868 | 1.00 | 38.21 | C |
| ATOM | 627 | CG | TRP | A | 82 | 18.624 | 17.489 | −5.441 | 1.00 | 37.72 | C |
| ATOM | 628 | CD1 | TRP | A | 82 | 17.979 | 18.408 | −6.215 | 1.00 | 37.00 | C |
| ATOM | 629 | NE1 | TRP | A | 82 | 17.745 | 19.554 | −5.501 | 1.00 | 32.60 | N |
| ATOM | 630 | CE2 | TRP | A | 82 | 18.244 | 19.407 | −4.238 | 1.00 | 33.03 | C |
| ATOM | 631 | CD2 | TRP | A | 82 | 18.812 | 18.116 | −4.153 | 1.00 | 37.58 | C |
| ATOM | 632 | CE3 | TRP | A | 82 | 19.389 | 17.718 | −2.945 | 1.00 | 33.91 | C |
| ATOM | 633 | CZ3 | TRP | A | 82 | 19.386 | 18.589 | −1.883 | 1.00 | 35.75 | C |
| ATOM | 634 | CH2 | TRP | A | 82 | 18.833 | 19.865 | −1.998 | 1.00 | 36.45 | C |
| ATOM | 635 | CZ2 | TRP | A | 82 | 18.267 | 20.298 | −3.175 | 1.00 | 34.18 | C |
| ATOM | 636 | C | TRP | A | 82 | 18.122 | 14.988 | −3.768 | 1.00 | 33.77 | C |
| ATOM | 637 | O | TRP | A | 82 | 19.003 | 14.408 | −3.102 | 1.00 | 31.33 | O |
| ATOM | 638 | N | PHE | A | 83 | 17.081 | 15.636 | −3.248 | 1.00 | 31.02 | N |
| ATOM | 639 | CA | PHE | A | 83 | 16.862 | 15.791 | −1.786 | 1.00 | 30.52 | C |
| ATOM | 640 | CB | PHE | A | 83 | 15.850 | 16.900 | −1.475 | 1.00 | 29.98 | C |
| ATOM | 641 | CG | PHE | A | 83 | 15.649 | 17.114 | −0.003 | 1.00 | 27.45 | C |
| ATOM | 642 | CD1 | PHE | A | 83 | 16.527 | 17.890 | 0.720 | 1.00 | 30.85 | C |
| ATOM | 643 | CE1 | PHE | A | 83 | 16.378 | 18.048 | 2.091 | 1.00 | 31.99 | C |
| ATOM | 644 | CZ | PHE | A | 83 | 15.380 | 17.382 | 2.755 | 1.00 | 31.31 | C |
| ATOM | 645 | CD2 | PHE | A | 83 | 14.644 | 16.454 | 0.674 | 1.00 | 29.02 | C |
| ATOM | 646 | CE2 | PHE | A | 83 | 14.512 | 16.583 | 2.048 | 1.00 | 31.35 | C |
| ATOM | 647 | C | PHE | A | 83 | 16.421 | 14.460 | −1.181 | 1.00 | 31.38 | C |
| ATOM | 648 | O | PHE | A | 83 | 16.749 | 14.202 | −0.019 | 1.00 | 38.68 | O |
| ATOM | 649 | N | VAL | A | 84 | 15.649 | 13.658 | −1.916 | 1.00 | 35.78 | N |
| ATOM | 650 | CA | VAL | A | 84 | 15.167 | 12.331 | −1.431 | 1.00 | 33.63 | C |
| ATOM | 651 | CB | VAL | A | 84 | 14.083 | 11.752 | −2.361 | 1.00 | 34.72 | C |
| ATOM | 652 | CG1 | VAL | A | 84 | 13.919 | 10.269 | −2.162 | 1.00 | 34.48 | C |
| ATOM | 653 | CG2 | VAL | A | 84 | 12.742 | 12.456 | −2.192 | 1.00 | 33.55 | C |
| ATOM | 654 | C | VAL | A | 84 | 16.385 | 11.408 | −1.289 | 1.00 | 31.56 | C |
| ATOM | 655 | O | VAL | A | 84 | 16.556 | 10.804 | −0.211 | 1.00 | 31.23 | O |
| ATOM | 656 | N | ALA | A | 85 | 17.213 | 11.339 | −2.322 | 1.00 | 28.69 | N |
| ATOM | 657 | CA | ALA | A | 85 | 18.521 | 10.643 | −2.299 | 1.00 | 33.22 | C |
| ATOM | 658 | CB | ALA | A | 85 | 19.322 | 11.007 | −3.513 | 1.00 | 34.20 | C |
| ATOM | 659 | C | ALA | A | 85 | 19.288 | 11.008 | −1.020 | 1.00 | 37.29 | C |
| ATOM | 660 | O | ALA | A | 85 | 19.707 | 10.068 | −0.263 | 1.00 | 41.53 | O |
| ATOM | 661 | N | ALA | A | 86 | 19.389 | 12.306 | −0.721 | 1.00 | 33.54 | N |
| ATOM | 662 | CA | ALA | A | 86 | 20.217 | 12.800 | 0.394 | 1.00 | 32.33 | C |
| ATOM | 663 | CB | ALA | A | 86 | 20.335 | 14.285 | 0.334 | 1.00 | 32.73 | C |
| ATOM | 664 | C | ALA | A | 86 | 19.641 | 12.311 | 1.719 | 1.00 | 31.87 | C |
| ATOM | 665 | O | ALA | A | 86 | 20.401 | 11.883 | 2.570 | 1.00 | 33.47 | O |
| ATOM | 666 | N | CYS | A | 87 | 18.329 | 12.343 | 1.887 | 1.00 | 35.81 | N |
| ATOM | 667 | CA | CYS | A | 87 | 17.680 | 11.859 | 3.131 | 1.00 | 34.64 | C |
| ATOM | 668 | CB | CYS | A | 87 | 16.214 | 12.251 | 3.193 | 1.00 | 34.99 | C |
| ATOM | 669 | SG | CYS | A | 87 | 15.990 | 14.029 | 3.360 | 1.00 | 37.46 | S |
| ATOM | 670 | C | CYS | A | 87 | 17.830 | 10.337 | 3.202 | 1.00 | 34.62 | C |
| ATOM | 671 | O | CYS | A | 87 | 17.938 | 9.816 | 4.308 | 1.00 | 33.50 | O |
| ATOM | 672 | N | SER | A | 88 | 17.896 | 9.646 | 2.067 | 1.00 | 35.44 | N |
| ATOM | 673 | CA | SER | A | 88 | 18.016 | 8.172 | 2.097 | 1.00 | 39.95 | C |
| ATOM | 674 | CB | SER | A | 88 | 17.804 | 7.544 | 0.791 | 1.00 | 39.78 | C |
| ATOM | 675 | OG | SER | A | 88 | 17.493 | 6.197 | 1.035 | 1.00 | 41.01 | O |
| ATOM | 676 | C | SER | A | 88 | 19.384 | 7.795 | 2.647 | 1.00 | 39.26 | C |
| ATOM | 677 | O | SER | A | 88 | 19.423 | 6.963 | 3.544 | 1.00 | 39.70 | O |
| ATOM | 678 | N | SER | A | 89 | 20.441 | 8.396 | 2.100 | 1.00 | 38.87 | N |
| ATOM | 679 | CA | SER | A | 89 | 21.834 | 8.285 | 2.597 | 1.00 | 36.81 | C |
| ATOM | 680 | CB | SER | A | 89 | 22.756 | 9.146 | 1.790 | 1.00 | 35.86 | C |
| ATOM | 681 | OG | SER | A | 89 | 22.813 | 8.687 | 0.445 | 1.00 | 39.05 | O |
| ATOM | 682 | C | SER | A | 89 | 21.855 | 8.654 | 4.081 | 1.00 | 38.06 | C |
| ATOM | 683 | O | SER | A | 89 | 22.370 | 7.874 | 4.858 | 1.00 | 40.00 | O |
| ATOM | 684 | N | LEU | A | 90 | 21.242 | 9.769 | 4.453 | 1.00 | 36.10 | N |
| ATOM | 685 | CA | LEU | A | 90 | 21.273 | 10.287 | 5.831 | 1.00 | 38.41 | C |
| ATOM | 686 | CB | LEU | A | 90 | 20.600 | 11.663 | 5.896 | 1.00 | 37.12 | C |
| ATOM | 687 | CG | LEU | A | 90 | 20.530 | 12.263 | 7.303 | 1.00 | 41.27 | C |
| ATOM | 688 | CD1 | LEU | A | 90 | 21.927 | 12.596 | 7.817 | 1.00 | 44.18 | C |
| ATOM | 689 | CD2 | LEU | A | 90 | 19.627 | 13.495 | 7.345 | 1.00 | 40.62 | C |
| ATOM | 690 | C | LEU | A | 90 | 20.603 | 9.270 | 6.759 | 1.00 | 43.63 | C |
| ATOM | 691 | O | LEU | A | 90 | 21.098 | 9.064 | 7.895 | 1.00 | 43.75 | O |
| ATOM | 692 | N | ALA | A | 91 | 19.519 | 8.649 | 6.309 | 1.00 | 45.21 | N |
| ATOM | 693 | CA | ALA | A | 91 | 18.789 | 7.632 | 7.099 | 1.00 | 43.03 | C |
| ATOM | 694 | CB | ALA | A | 91 | 17.465 | 7.325 | 6.440 | 1.00 | 45.84 | C |
| ATOM | 695 | C | ALA | A | 91 | 19.650 | 6.376 | 7.279 | 1.00 | 39.41 | C |
| ATOM | 696 | O | ALA | A | 91 | 19.422 | 5.697 | 8.261 | 1.00 | 40.81 | O |
| ATOM | 697 | N | SER | A | 92 | 20.583 | 6.098 | 6.355 | 1.00 | 42.83 | N |
| ATOM | 698 | CA | SER | A | 92 | 21.541 | 4.955 | 6.341 | 1.00 | 45.09 | C |
| ATOM | 699 | CB | SER | A | 92 | 22.688 | 5.202 | 5.423 | 1.00 | 42.98 | C |
| ATOM | 700 | OG | SER | A | 92 | 22.325 | 4.945 | 4.094 | 1.00 | 49.04 | O |
| ATOM | 701 | C | SER | A | 92 | 22.144 | 4.678 | 7.715 | 1.00 | 52.50 | C |
| ATOM | 702 | O | SER | A | 92 | 22.277 | 3.506 | 8.029 | 1.00 | 60.64 | O |
| ATOM | 703 | N | ARG | A | 93 | 22.571 | 5.718 | 8.442 | 1.00 | 53.09 | N |
| ATOM | 704 | CA | ARG | A | 93 | 23.446 | 5.607 | 9.639 | 1.00 | 53.57 | C |
| ATOM | 705 | CB | ARG | A | 93 | 24.841 | 6.125 | 9.285 | 1.00 | 61.39 | C |
| ATOM | 706 | CG | ARG | A | 93 | 25.535 | 5.325 | 8.197 | 1.00 | 60.55 | C |
| ATOM | 707 | CD | ARG | A | 93 | 27.004 | 5.657 | 8.197 | 1.00 | 62.25 | C |
| ATOM | 708 | NE | ARG | A | 93 | 27.589 | 5.196 | 6.952 | 1.00 | 64.70 | N |
| ATOM | 709 | CZ | ARG | A | 93 | 28.399 | 4.151 | 6.831 | 1.00 | 67.86 | C |
| ATOM | 710 | NH1 | ARG | A | 93 | 28.769 | 3.455 | 7.897 | 1.00 | 65.82 | N |
| ATOM | 711 | NH2 | ARG | A | 93 | 28.855 | 3.817 | 5.638 | 1.00 | 68.39 | N |
| ATOM | 712 | C | ARG | A | 93 | 22.904 | 6.408 | 10.829 | 1.00 | 49.55 | C |
| ATOM | 713 | O | ARG | A | 93 | 22.936 | 7.655 | 10.764 | 1.00 | 47.07 | O |
| ATOM | 714 | N | GLU | A | 94 | 22.531 | 5.711 | 11.911 | 1.00 | 50.38 | N |
| ATOM | 715 | CA | GLU | A | 94 | 22.090 | 6.289 | 13.208 | 1.00 | 54.33 | C |
| ATOM | 716 | CB | GLU | A | 94 | 22.246 | 5.284 | 14.348 | 1.00 | 66.83 | C |
| ATOM | 717 | CG | GLU | A | 94 | 20.933 | 4.643 | 14.761 | 1.00 | 77.23 | C |
| ATOM | 718 | CD | GLU | A | 94 | 20.687 | 4.593 | 16.259 | 1.00 | 80.87 | C |
| ATOM | 719 | OE1 | GLU | A | 94 | 21.578 | 5.040 | 17.028 | 1.00 | 77.66 | O |
| ATOM | 720 | OE2 | GLU | A | 94 | 19.600 | 4.101 | 16.650 | 1.00 | 79.70 | O |
| ATOM | 721 | C | GLU | A | 94 | 22.898 | 7.532 | 13.588 | 1.00 | 53.96 | C |
| ATOM | 722 | O | GLU | A | 94 | 22.267 | 8.527 | 13.944 | 1.00 | 52.75 | O |
| ATOM | 723 | N | SER | A | 95 | 24.233 | 7.477 | 13.569 | 1.00 | 48.88 | N |
| ATOM | 724 | CA | SER | A | 95 | 25.070 | 8.553 | 14.153 | 1.00 | 53.61 | C |
| ATOM | 725 | CB | SER | A | 95 | 26.470 | 8.084 | 14.478 | 1.00 | 55.99 | C |
| ATOM | 726 | OG | SER | A | 95 | 27.289 | 8.032 | 13.328 | 1.00 | 64.25 | O |
| ATOM | 727 | C | SER | A | 95 | 25.043 | 9.786 | 13.242 | 1.00 | 52.43 | C |
| ATOM | 728 | O | SER | A | 95 | 25.448 | 10.881 | 13.709 | 1.00 | 54.09 | O |
| ATOM | 729 | N | LEU | A | 96 | 24.555 | 9.646 | 12.008 | 1.00 | 48.56 | N |
| ATOM | 730 | CA | LEU | A | 96 | 24.514 | 10.783 | 11.058 | 1.00 | 44.31 | C |
| ATOM | 731 | CB | LEU | A | 96 | 24.873 | 10.295 | 9.652 | 1.00 | 44.41 | C |
| ATOM | 732 | CG | LEU | A | 96 | 26.324 | 9.828 | 9.474 | 1.00 | 47.16 | C |
| ATOM | 733 | CD1 | LEU | A | 96 | 26.666 | 9.631 | 8.006 | 1.00 | 48.01 | C |
| ATOM | 734 | CD2 | LEU | A | 96 | 27.333 | 10.799 | 10.085 | 1.00 | 48.11 | C |
| ATOM | 735 | C | LEU | A | 96 | 23.149 | 11.474 | 11.152 | 1.00 | 43.53 | C |
| ATOM | 736 | O | LEU | A | 96 | 23.155 | 12.699 | 11.332 | 1.00 | 49.99 | O |
| ATOM | 737 | N | TRP | A | 97 | 22.024 | 10.748 | 11.100 | 1.00 | 45.69 | N |
| ATOM | 738 | CA | TRP | A | 97 | 20.678 | 11.391 | 11.080 | 1.00 | 45.09 | C |
| ATOM | 739 | CB | TRP | A | 97 | 19.573 | 10.484 | 10.516 | 1.00 | 45.49 | C |
| ATOM | 740 | CG | TRP | A | 97 | 19.158 | 9.316 | 11.361 | 1.00 | 46.58 | C |
| ATOM | 741 | CD1 | TRP | A | 97 | 19.505 | 8.012 | 11.164 | 1.00 | 47.91 | C |
| ATOM | 742 | NE1 | TRP | A | 97 | 18.920 | 7.209 | 12.107 | 1.00 | 47.58 | N |
| ATOM | 743 | CE2 | TRP | A | 97 | 18.151 | 7.978 | 12.933 | 1.00 | 47.28 | C |
| ATOM | 744 | CD2 | TRP | A | 97 | 18.256 | 9.315 | 12.486 | 1.00 | 48.67 | C |
| ATOM | 745 | CE3 | TRP | A | 97 | 17.546 | 10.305 | 13.179 | 1.00 | 47.21 | C |
| ATOM | 746 | CZ3 | TRP | A | 97 | 16.781 | 9.940 | 14.267 | 1.00 | 43.59 | C |
| ATOM | 747 | CH2 | TRP | A | 97 | 16.704 | 8.616 | 14.690 | 1.00 | 45.21 | C |
| ATOM | 748 | CZ2 | TRP | A | 97 | 17.377 | 7.613 | 14.033 | 1.00 | 47.82 | C |
| ATOM | 749 | C | TRP | A | 97 | 20.357 | 11.897 | 12.486 | 1.00 | 45.05 | C |
| ATOM | 750 | O | TRP | A | 97 | 19.603 | 12.865 | 12.606 | 1.00 | 47.06 | O |
| ATOM | 751 | N | GLN | A | 98 | 20.922 | 11.270 | 13.510 | 1.00 | 46.02 | N |
| ATOM | 752 | CA | GLN | A | 98 | 20.788 | 11.738 | 14.907 | 1.00 | 50.20 | C |
| ATOM | 753 | CB | GLN | A | 98 | 21.103 | 10.617 | 15.896 | 1.00 | 55.65 | C |
| ATOM | 754 | CG | GLN | A | 98 | 20.039 | 9.528 | 15.939 | 1.00 | 56.73 | C |
| ATOM | 755 | CD | GLN | A | 98 | 20.223 | 8.630 | 17.139 | 1.00 | 55.71 | C |
| ATOM | 756 | OE1 | GLN | A | 98 | 21.327 | 8.444 | 17.647 | 1.00 | 53.05 | O |
| ATOM | 757 | NE2 | GLN | A | 98 | 19.129 | 8.062 | 17.607 | 1.00 | 59.06 | N |
| ATOM | 758 | C | GLN | A | 98 | 21.666 | 12.973 | 15.160 | 1.00 | 49.77 | C |
| ATOM | 759 | O | GLN | A | 98 | 21.436 | 13.573 | 16.204 | 1.00 | 58.41 | O |
| ATOM | 760 | N | LYS | A | 99 | 22.605 | 13.357 | 14.281 | 1.00 | 47.04 | N |
| ATOM | 761 | CA | LYS | A | 99 | 23.266 | 14.693 | 14.378 | 1.00 | 50.24 | C |
| ATOM | 762 | CB | LYS | A | 99 | 24.636 | 14.781 | 13.698 | 1.00 | 57.65 | C |
| ATOM | 763 | CG | LYS | A | 99 | 25.792 | 14.118 | 14.443 | 1.00 | 70.52 | C |
| ATOM | 764 | CD | LYS | A | 99 | 25.679 | 14.055 | 15.973 | 1.00 | 76.62 | C |
| ATOM | 765 | CE | LYS | A | 99 | 26.705 | 13.139 | 16.617 | 1.00 | 79.18 | C |
| ATOM | 766 | NZ | LYS | A | 99 | 26.318 | 11.706 | 16.531 | 1.00 | 77.51 | N |
| ATOM | 767 | C | LYS | A | 99 | 22.364 | 15.745 | 13.743 | 1.00 | 47.33 | C |
| ATOM | 768 | O | LYS | A | 99 | 22.269 | 16.844 | 14.287 | 1.00 | 50.09 | O |
| ATOM | 769 | N | VAL | A | 100 | 21.713 | 15.413 | 12.633 | 1.00 | 44.04 | N |
| ATOM | 770 | CA | VAL | A | 100 | 20.829 | 16.370 | 11.920 | 1.00 | 38.17 | C |
| ATOM | 771 | CB | VAL | A | 100 | 20.581 | 15.914 | 10.481 | 1.00 | 33.60 | C |
| ATOM | 772 | CG1 | VAL | A | 100 | 19.450 | 16.699 | 9.852 | 1.00 | 32.01 | C |
| ATOM | 773 | CG2 | VAL | A | 100 | 21.873 | 16.040 | 9.682 | 1.00 | 31.46 | C |
| ATOM | 774 | C | VAL | A | 100 | 19.540 | 16.562 | 12.710 | 1.00 | 38.83 | C |
| ATOM | 775 | O | VAL | A | 100 | 19.037 | 17.690 | 12.742 | 1.00 | 39.03 | O |
| ATOM | 776 | N | ILE | A | 101 | 19.044 | 15.501 | 13.335 | 1.00 | 41.88 | N |
| ATOM | 777 | CA | ILE | A | 101 | 17.766 | 15.519 | 14.092 | 1.00 | 43.00 | C |
| ATOM | 778 | CB | ILE | A | 101 | 16.743 | 14.536 | 13.506 | 1.00 | 42.08 | C |
| ATOM | 779 | CG1 | ILE | A | 101 | 16.635 | 14.689 | 11.987 | 1.00 | 44.18 | C |
| ATOM | 780 | CG2 | ILE | A | 101 | 15.406 | 14.729 | 14.194 | 1.00 | 42.11 | C |
| ATOM | 781 | CD1 | ILE | A | 101 | 15.755 | 13.667 | 11.317 | 1.00 | 49.53 | C |
| ATOM | 782 | C | ILE | A | 101 | 18.110 | 15.227 | 15.542 | 1.00 | 48.82 | C |
| ATOM | 783 | O | ILE | A | 101 | 17.980 | 14.097 | 16.015 | 1.00 | 49.96 | O |
| ATOM | 784 | N | PRO | A | 102 | 18.539 | 16.265 | 16.288 | 1.00 | 48.30 | N |
| ATOM | 785 | CA | PRO | A | 102 | 19.043 | 16.074 | 17.644 | 1.00 | 49.20 | C |
| ATOM | 786 | CB | PRO | A | 102 | 19.644 | 17.450 | 17.959 | 1.00 | 50.14 | C |
| ATOM | 787 | CG | PRO | A | 102 | 18.769 | 18.409 | 17.186 | 1.00 | 49.08 | C |
| ATOM | 788 | CD | PRO | A | 102 | 18.485 | 17.680 | 15.891 | 1.00 | 46.64 | C |
| ATOM | 789 | C | PRO | A | 102 | 17.931 | 15.697 | 18.633 | 1.00 | 52.08 | C |
| ATOM | 790 | O | PRO | A | 102 | 16.797 | 16.112 | 18.451 | 1.00 | 52.27 | O |
| ATOM | 791 | N | ASP | A | 103 | 18.270 | 14.892 | 19.640 | 1.00 | 57.29 | N |
| ATOM | 792 | CA | ASP | A | 103 | 17.366 | 14.603 | 20.781 | 1.00 | 60.72 | C |
| ATOM | 793 | CB | ASP | A | 103 | 17.264 | 15.817 | 21.710 | 1.00 | 70.01 | C |
| ATOM | 794 | CG | ASP | A | 103 | 18.599 | 16.475 | 22.033 | 1.00 | 80.72 | C |
| ATOM | 795 | OD1 | ASP | A | 103 | 19.564 | 15.735 | 22.345 | 1.00 | 86.23 | O |
| ATOM | 796 | OD2 | ASP | A | 103 | 18.672 | 17.730 | 21.961 | 1.00 | 89.89 | O |
| ATOM | 797 | C | ASP | A | 103 | 16.007 | 14.243 | 20.190 | 1.00 | 57.13 | C |
| ATOM | 798 | O | ASP | A | 103 | 15.012 | 14.884 | 20.558 | 1.00 | 55.16 | O |
| ATOM | 799 | N | TRP | A | 104 | 15.996 | 13.292 | 19.255 | 1.00 | 57.52 | N |
| ATOM | 800 | CA | TRP | A | 104 | 14.828 | 13.023 | 18.381 | 1.00 | 58.28 | C |
| ATOM | 801 | CB | TRP | A | 104 | 15.159 | 12.030 | 17.260 | 1.00 | 62.72 | C |
| ATOM | 802 | CG | TRP | A | 104 | 15.219 | 10.585 | 17.645 | 1.00 | 67.63 | C |
| ATOM | 803 | CD1 | TRP | A | 104 | 16.340 | 9.850 | 17.905 | 1.00 | 71.64 | C |
| ATOM | 804 | NE1 | TRP | A | 104 | 16.004 | 8.551 | 18.183 | 1.00 | 72.35 | N |
| ATOM | 805 | CE2 | TRP | A | 104 | 14.645 | 8.410 | 18.092 | 1.00 | 73.79 | C |
| ATOM | 806 | CD2 | TRP | A | 104 | 14.112 | 9.672 | 17.746 | 1.00 | 69.41 | C |
| ATOM | 807 | CE3 | TRP | A | 104 | 12.728 | 9.793 | 17.587 | 1.00 | 68.85 | C |
| ATOM | 808 | CZ3 | TRP | A | 104 | 11.935 | 8.679 | 17.771 | 1.00 | 73.97 | C |
| ATOM | 809 | CH2 | TRP | A | 104 | 12.485 | 7.442 | 18.118 | 1.00 | 75.74 | C |
| ATOM | 810 | CZ2 | TRP | A | 104 | 13.843 | 7.283 | 18.282 | 1.00 | 76.69 | C |
| ATOM | 811 | C | TRP | A | 104 | 13.643 | 12.575 | 19.228 | 1.00 | 53.97 | C |
| ATOM | 812 | O | TRP | A | 104 | 12.529 | 12.859 | 18.827 | 1.00 | 54.76 | O |
| ATOM | 813 | N | LYS | A | 105 | 13.877 | 11.933 | 20.366 | 1.00 | 60.52 | N |
| ATOM | 814 | CA | LYS | A | 105 | 12.778 | 11.451 | 21.248 | 1.00 | 67.56 | C |
| ATOM | 815 | CB | LYS | A | 105 | 13.247 | 10.319 | 22.171 | 1.00 | 71.04 | C |
| ATOM | 816 | CG | LYS | A | 105 | 13.438 | 8.977 | 21.471 | 1.00 | 77.15 | C |
| ATOM | 817 | CD | LYS | A | 105 | 13.945 | 7.890 | 22.388 | 1.00 | 84.09 | C |
| ATOM | 818 | CE | LYS | A | 105 | 14.546 | 6.711 | 21.653 | 1.00 | 90.38 | C |
| ATOM | 819 | NZ | LYS | A | 105 | 15.470 | 5.945 | 22.527 | 1.00 | 96.17 | N |
| ATOM | 820 | C | LYS | A | 105 | 12.185 | 12.640 | 22.011 | 1.00 | 60.68 | C |
| ATOM | 821 | O | LYS | A | 105 | 11.008 | 12.551 | 22.396 | 1.00 | 66.47 | O |
| ATOM | 822 | N | GLU | A | 106 | 12.948 | 13.719 | 22.172 | 1.00 | 61.77 | N |
| ATOM | 823 | CA | GLU | A | 106 | 12.508 | 14.972 | 22.850 | 1.00 | 64.23 | C |
| ATOM | 824 | CB | GLU | A | 106 | 13.736 | 15.741 | 23.361 | 1.00 | 72.03 | C |
| ATOM | 825 | CG | GLU | A | 106 | 13.431 | 17.072 | 24.037 | 1.00 | 82.57 | C |
| ATOM | 826 | CD | GLU | A | 106 | 13.020 | 17.022 | 25.504 | 1.00 | 87.19 | C |
| ATOM | 827 | OE1 | GLU | A | 106 | 13.378 | 16.040 | 26.186 | 1.00 | 85.49 | O |
| ATOM | 828 | OE2 | GLU | A | 106 | 12.340 | 17.971 | 25.964 | 1.00 | 87.52 | O |
| ATOM | 829 | C | GLU | A | 106 | 11.634 | 15.788 | 21.883 | 1.00 | 59.98 | C |
| ATOM | 830 | O | GLU | A | 106 | 10.839 | 16.616 | 22.377 | 1.00 | 58.59 | O |
| ATOM | 831 | N | GLN | A | 107 | 11.754 | 15.556 | 20.567 | 1.00 | 49.55 | N |
| ATOM | 832 | CA | GLN | A | 107 | 10.967 | 16.272 | 19.532 | 1.00 | 47.99 | C |
| ATOM | 833 | CB | GLN | A | 107 | 11.835 | 16.565 | 18.308 | 1.00 | 48.36 | C |
| ATOM | 834 | CG | GLN | A | 107 | 13.117 | 17.313 | 18.641 | 1.00 | 46.26 | C |
| ATOM | 835 | CD | GLN | A | 107 | 13.536 | 18.257 | 17.543 | 1.00 | 43.18 | C |
| ATOM | 836 | OE1 | GLN | A | 107 | 12.757 | 19.072 | 17.068 | 1.00 | 42.51 | O |
| ATOM | 837 | NE2 | GLN | A | 107 | 14.783 | 18.157 | 17.124 | 1.00 | 45.12 | N |
| ATOM | 838 | C | GLN | A | 107 | 9.735 | 15.456 | 19.129 | 1.00 | 47.34 | C |
| ATOM | 839 | O | GLN | A | 107 | 8.720 | 16.058 | 18.745 | 1.00 | 51.61 | O |
| ATOM | 840 | N | GLU | A | 108 | 9.832 | 14.133 | 19.168 | 1.00 | 52.01 | N |
| ATOM | 841 | CA | GLU | A | 108 | 8.795 | 13.208 | 18.635 | 1.00 | 53.30 | C |
| ATOM | 842 | CB | GLU | A | 108 | 9.279 | 11.759 | 18.792 | 1.00 | 52.31 | C |
| ATOM | 843 | CG | GLU | A | 108 | 8.294 | 10.691 | 18.326 | 1.00 | 54.73 | C |
| ATOM | 844 | CD | GLU | A | 108 | 7.867 | 10.754 | 16.863 | 1.00 | 53.56 | C |
| ATOM | 845 | OE1 | GLU | A | 108 | 7.167 | 11.711 | 16.498 | 1.00 | 54.71 | O |
| ATOM | 846 | OE2 | GLU | A | 108 | 8.252 | 9.855 | 16.083 | 1.00 | 53.35 | O |
| ATOM | 847 | C | GLU | A | 108 | 7.472 | 13.485 | 19.363 | 1.00 | 50.05 | C |
| ATOM | 848 | O | GLU | A | 108 | 7.508 | 13.827 | 20.545 | 1.00 | 56.05 | O |
| ATOM | 849 | N | TRP | A | 109 | 6.344 | 13.385 | 18.669 | 1.00 | 49.43 | N |
| ATOM | 850 | CA | TRP | A | 109 | 5.009 | 13.439 | 19.312 | 1.00 | 47.91 | C |
| ATOM | 851 | CB | TRP | A | 109 | 3.881 | 13.364 | 18.277 | 1.00 | 46.34 | C |
| ATOM | 852 | CG | TRP | A | 109 | 3.666 | 14.614 | 17.483 | 1.00 | 49.37 | C |
| ATOM | 853 | CD1 | TRP | A | 109 | 2.811 | 15.635 | 17.784 | 1.00 | 50.86 | C |
| ATOM | 854 | NE1 | TRP | A | 109 | 2.872 | 16.610 | 16.822 | 1.00 | 51.16 | N |
| ATOM | 855 | CE2 | TRP | A | 109 | 3.779 | 16.241 | 15.863 | 1.00 | 48.82 | C |
| ATOM | 856 | CD2 | TRP | A | 109 | 4.300 | 14.982 | 16.239 | 1.00 | 50.74 | C |
| ATOM | 857 | CE3 | TRP | A | 109 | 5.253 | 14.381 | 15.411 | 1.00 | 47.02 | C |
| ATOM | 858 | CZ3 | TRP | A | 109 | 5.641 | 15.036 | 14.264 | 1.00 | 47.87 | C |
| ATOM | 859 | CH2 | TRP | A | 109 | 5.106 | 16.273 | 13.910 | 1.00 | 45.96 | C |
| ATOM | 860 | CZ2 | TRP | A | 109 | 4.175 | 16.901 | 14.700 | 1.00 | 45.50 | C |
| ATOM | 861 | C | TRP | A | 109 | 4.931 | 12.281 | 20.306 | 1.00 | 50.28 | C |
| ATOM | 862 | O | TRP | A | 109 | 5.365 | 11.165 | 19.968 | 1.00 | 49.48 | O |
| ATOM | 863 | N | ASP | A | 110 | 4.420 | 12.539 | 21.503 | 1.00 | 56.02 | N |
| ATOM | 864 | CA | ASP | A | 110 | 4.049 | 11.474 | 22.466 | 1.00 | 57.55 | C |
| ATOM | 865 | CB | ASP | A | 110 | 4.974 | 11.443 | 23.677 | 1.00 | 62.62 | C |
| ATOM | 866 | CG | ASP | A | 110 | 4.616 | 10.352 | 24.661 | 1.00 | 63.71 | C |
| ATOM | 867 | OD1 | ASP | A | 110 | 4.629 | 9.169 | 24.269 | 1.00 | 73.83 | O |
| ATOM | 868 | OD2 | ASP | A | 110 | 4.290 | 10.700 | 25.796 | 1.00 | 72.44 | O |
| ATOM | 869 | C | ASP | A | 110 | 2.604 | 11.724 | 22.852 | 1.00 | 63.67 | C |
| ATOM | 870 | O | ASP | A | 110 | 2.302 | 12.736 | 23.475 | 1.00 | 65.23 | O |
| ATOM | 871 | N | PRO | A | 111 | 1.669 | 10.847 | 22.431 | 1.00 | 72.19 | N |
| ATOM | 872 | CA | PRO | A | 111 | 0.255 | 11.032 | 22.751 | 1.00 | 74.66 | C |
| ATOM | 873 | CB | PRO | A | 111 | −0.459 | 9.965 | 21.905 | 1.00 | 79.61 | C |
| ATOM | 874 | CG | PRO | A | 111 | 0.599 | 8.900 | 21.666 | 1.00 | 83.22 | C |
| ATOM | 875 | CD | PRO | A | 111 | 1.919 | 9.646 | 21.615 | 1.00 | 78.98 | C |
| ATOM | 876 | C | PRO | A | 111 | −0.054 | 10.872 | 24.250 | 1.00 | 70.95 | C |
| ATOM | 877 | O | PRO | A | 111 | −1.103 | 11.320 | 24.623 | 1.00 | 77.98 | O |
| ATOM | 878 | N | GLU | A | 112 | 0.851 | 10.299 | 25.055 | 1.00 | 65.70 | N |
| ATOM | 879 | CA | GLU | A | 112 | 0.714 | 10.241 | 26.541 | 1.00 | 73.63 | C |
| ATOM | 880 | CB | GLU | A | 112 | 1.568 | 9.132 | 27.161 | 1.00 | 76.61 | C |
| ATOM | 881 | CG | GLU | A | 112 | 1.404 | 7.778 | 26.495 | 1.00 | 86.24 | C |
| ATOM | 882 | CD | GLU | A | 112 | 1.672 | 6.583 | 27.400 | 1.00 | 89.53 | C |
| ATOM | 883 | OE1 | GLU | A | 112 | 0.710 | 6.109 | 28.040 | 1.00 | 92.55 | O |
| ATOM | 884 | OE2 | GLU | A | 112 | 2.840 | 6.131 | 27.471 | 1.00 | 81.09 | O |
| ATOM | 885 | C | GLU | A | 112 | 1.091 | 11.590 | 27.180 | 1.00 | 76.68 | C |
| ATOM | 886 | O | GLU | A | 112 | 0.704 | 11.804 | 28.347 | 1.00 | 77.57 | O |
| ATOM | 887 | N | LYS | A | 113 | 1.809 | 12.466 | 26.466 | 1.00 | 75.36 | N |
| ATOM | 888 | CA | LYS | A | 113 | 2.160 | 13.836 | 26.937 | 1.00 | 73.68 | C |
| ATOM | 889 | CB | LYS | A | 113 | 3.680 | 13.995 | 27.094 | 1.00 | 68.85 | C |
| ATOM | 890 | CG | LYS | A | 113 | 4.354 | 13.116 | 28.143 | 1.00 | 70.79 | C |
| ATOM | 891 | CD | LYS | A | 113 | 5.876 | 12.996 | 27.992 | 1.00 | 69.46 | C |
| ATOM | 892 | CE | LYS | A | 113 | 6.675 | 13.863 | 28.945 | 1.00 | 71.46 | C |
| ATOM | 893 | NZ | LYS | A | 113 | 6.233 | 15.278 | 28.910 | 1.00 | 78.31 | N |
| ATOM | 894 | C | LYS | A | 113 | 1.623 | 14.858 | 25.936 | 1.00 | 76.01 | C |
| ATOM | 895 | O | LYS | A | 113 | 2.396 | 15.575 | 25.319 | 1.00 | 77.54 | O |
| ATOM | 896 | N | PRO | A | 114 | 0.293 | 14.990 | 25.739 | 1.00 | 82.43 | N |
| ATOM | 897 | CA | PRO | A | 114 | −0.246 | 15.863 | 24.689 | 1.00 | 82.87 | C |
| ATOM | 898 | CB | PRO | A | 114 | −1.766 | 15.755 | 24.883 | 1.00 | 90.43 | C |
| ATOM | 899 | CG | PRO | A | 114 | −1.956 | 14.414 | 25.561 | 1.00 | 94.31 | C |
| ATOM | 900 | CD | PRO | A | 114 | −0.762 | 14.290 | 26.486 | 1.00 | 91.12 | C |
| ATOM | 901 | C | PRO | A | 114 | 0.150 | 17.346 | 24.767 | 1.00 | 78.92 | C |
| ATOM | 902 | O | PRO | A | 114 | 0.391 | 17.952 | 23.725 | 1.00 | 79.81 | O |
| ATOM | 903 | N | ASN | A | 115 | 0.202 | 17.897 | 25.980 | 1.00 | 73.76 | N |
| ATOM | 904 | CA | ASN | A | 115 | 0.526 | 19.326 | 26.230 | 1.00 | 74.10 | C |
| ATOM | 905 | CB | ASN | A | 115 | 0.200 | 19.734 | 27.670 | 1.00 | 82.68 | C |
| ATOM | 906 | CG | ASN | A | 115 | −1.270 | 20.042 | 27.864 | 1.00 | 82.82 | C |
| ATOM | 907 | OD1 | ASN | A | 115 | −2.018 | 20.153 | 26.891 | 1.00 | 88.59 | O |
| ATOM | 908 | ND2 | ASN | A | 115 | −1.690 | 20.196 | 29.111 | 1.00 | 82.29 | N |
| ATOM | 909 | C | ASN | A | 115 | 1.993 | 19.611 | 25.887 | 1.00 | 68.82 | C |
| ATOM | 910 | O | ASN | A | 115 | 2.307 | 20.797 | 25.674 | 1.00 | 65.60 | O |
| ATOM | 911 | N | ALA | A | 116 | 2.848 | 18.582 | 25.830 | 1.00 | 60.73 | N |
| ATOM | 912 | CA | ALA | A | 116 | 4.250 | 18.676 | 25.351 | 1.00 | 61.79 | C |
| ATOM | 913 | CB | ALA | A | 116 | 4.908 | 17.321 | 25.332 | 1.00 | 59.48 | C |
| ATOM | 914 | C | ALA | A | 116 | 4.316 | 19.319 | 23.957 | 1.00 | 62.13 | C |
| ATOM | 915 | O | ALA | A | 116 | 5.267 | 20.078 | 23.714 | 1.00 | 61.02 | O |
| ATOM | 916 | N | TYR | A | 117 | 3.382 | 19.010 | 23.055 | 1.00 | 62.70 | N |
| ATOM | 917 | CA | TYR | A | 117 | 3.427 | 19.490 | 21.647 | 1.00 | 62.04 | C |
| ATOM | 918 | CB | TYR | A | 117 | 2.288 | 18.906 | 20.815 | 1.00 | 57.03 | C |
| ATOM | 919 | CG | TYR | A | 117 | 2.266 | 19.448 | 19.413 | 1.00 | 56.47 | C |
| ATOM | 920 | CD1 | TYR | A | 117 | 3.381 | 19.343 | 18.587 | 1.00 | 57.12 | C |
| ATOM | 921 | CE1 | TYR | A | 117 | 3.372 | 19.833 | 17.288 | 1.00 | 52.79 | C |
| ATOM | 922 | CZ | TYR | A | 117 | 2.236 | 20.451 | 16.791 | 1.00 | 52.53 | C |
| ATOM | 923 | OH | TYR | A | 117 | 2.201 | 20.953 | 15.516 | 1.00 | 47.95 | O |
| ATOM | 924 | CE2 | TYR | A | 117 | 1.120 | 20.566 | 17.603 | 1.00 | 55.55 | C |
| ATOM | 925 | CD2 | TYR | A | 117 | 1.140 | 20.067 | 18.900 | 1.00 | 54.67 | C |
| ATOM | 926 | C | TYR | A | 117 | 3.316 | 21.019 | 21.594 | 1.00 | 61.59 | C |
| ATOM | 927 | O | TYR | A | 117 | 2.373 | 21.548 | 22.201 | 1.00 | 62.92 | O |
| ATOM | 928 | N | ALA | A | 118 | 4.206 | 21.692 | 20.852 | 1.00 | 56.40 | N |
| ATOM | 929 | CA | ALA | A | 118 | 4.196 | 23.166 | 20.687 | 1.00 | 55.89 | C |
| ATOM | 930 | CB | ALA | A | 118 | 5.189 | 23.786 | 21.632 | 1.00 | 54.71 | C |
| ATOM | 931 | C | ALA | A | 118 | 4.473 | 23.593 | 19.239 | 1.00 | 55.07 | C |
| ATOM | 932 | O | ALA | A | 118 | 4.664 | 24.804 | 19.022 | 1.00 | 53.94 | O |
| ATOM | 933 | N | GLY | A | 119 | 4.441 | 22.665 | 18.281 | 1.00 | 53.18 | N |
| ATOM | 934 | CA | GLY | A | 119 | 4.515 | 22.984 | 16.840 | 1.00 | 52.65 | C |
| ATOM | 935 | C | GLY | A | 119 | 5.848 | 23.608 | 16.469 | 1.00 | 49.73 | C |
| ATOM | 936 | O | GLY | A | 119 | 5.873 | 24.474 | 15.564 | 1.00 | 50.15 | O |
| ATOM | 937 | N | ILE | A | 120 | 6.924 | 23.149 | 17.104 | 1.00 | 45.15 | N |
| ATOM | 938 | CA | ILE | A | 120 | 8.297 | 23.647 | 16.838 | 1.00 | 45.02 | C |
| ATOM | 939 | CB | ILE | A | 120 | 8.676 | 24.644 | 17.937 | 1.00 | 47.64 | C |
| ATOM | 940 | CG1 | ILE | A | 120 | 10.144 | 25.066 | 17.861 | 1.00 | 53.54 | C |
| ATOM | 941 | CG2 | ILE | A | 120 | 8.294 | 24.101 | 19.299 | 1.00 | 52.72 | C |
| ATOM | 942 | CD1 | ILE | A | 120 | 10.452 | 26.335 | 18.644 | 1.00 | 58.04 | C |
| ATOM | 943 | C | ILE | A | 120 | 9.267 | 22.467 | 16.704 | 1.00 | 44.92 | C |
| ATOM | 944 | O | ILE | A | 120 | 9.081 | 21.435 | 17.358 | 1.00 | 42.64 | O |
| ATOM | 945 | N | PHE | A | 121 | 10.262 | 22.613 | 15.832 | 1.00 | 47.53 | N |
| ATOM | 946 | CA | PHE | A | 121 | 11.320 | 21.605 | 15.574 | 1.00 | 47.13 | C |
| ATOM | 947 | CB | PHE | A | 121 | 10.920 | 20.736 | 14.378 | 1.00 | 47.84 | C |
| ATOM | 948 | CG | PHE | A | 121 | 9.789 | 19.783 | 14.678 | 1.00 | 46.05 | C |
| ATOM | 949 | CD1 | PHE | A | 121 | 10.043 | 18.540 | 15.231 | 1.00 | 42.38 | C |
| ATOM | 950 | CE1 | PHE | A | 121 | 9.013 | 17.663 | 15.529 | 1.00 | 38.41 | C |
| ATOM | 951 | CZ | PHE | A | 121 | 7.719 | 18.022 | 15.282 | 1.00 | 41.67 | C |
| ATOM | 952 | CD2 | PHE | A | 121 | 8.473 | 20.133 | 14.427 | 1.00 | 44.25 | C |
| ATOM | 953 | CE2 | PHE | A | 121 | 7.446 | 19.259 | 14.743 | 1.00 | 45.20 | C |
| ATOM | 954 | C | PHE | A | 121 | 12.643 | 22.338 | 15.361 | 1.00 | 45.39 | C |
| ATOM | 955 | O | PHE | A | 121 | 12.623 | 23.554 | 15.087 | 1.00 | 50.67 | O |
| ATOM | 956 | N | HIS | A | 122 | 13.757 | 21.621 | 15.471 | 1.00 | 41.45 | N |
| ATOM | 957 | CA | HIS | A | 122 | 15.112 | 22.181 | 15.249 | 1.00 | 45.94 | C |
| ATOM | 958 | CB | HIS | A | 122 | 15.658 | 22.864 | 16.520 | 1.00 | 49.10 | C |
| ATOM | 959 | CG | HIS | A | 122 | 16.115 | 21.947 | 17.604 | 1.00 | 47.84 | C |
| ATOM | 960 | ND1 | HIS | A | 122 | 17.423 | 21.913 | 18.021 | 1.00 | 46.54 | N |
| ATOM | 961 | CE1 | HIS | A | 122 | 17.550 | 21.030 | 18.996 | 1.00 | 49.02 | C |
| ATOM | 962 | NE2 | HIS | A | 122 | 16.350 | 20.491 | 19.235 | 1.00 | 50.95 | N |
| ATOM | 963 | CD2 | HIS | A | 122 | 15.444 | 21.064 | 18.376 | 1.00 | 49.76 | C |
| ATOM | 964 | C | HIS | A | 122 | 16.022 | 21.085 | 14.699 | 1.00 | 46.14 | C |
| ATOM | 965 | O | HIS | A | 122 | 15.836 | 19.911 | 15.065 | 1.00 | 50.75 | O |
| ATOM | 966 | N | PHE | A | 123 | 16.943 | 21.480 | 13.824 | 1.00 | 42.37 | N |
| ATOM | 967 | CA | PHE | A | 123 | 17.851 | 20.588 | 13.069 | 1.00 | 41.95 | C |
| ATOM | 968 | CB | PHE | A | 123 | 17.263 | 20.320 | 11.684 | 1.00 | 40.01 | C |
| ATOM | 969 | CG | PHE | A | 123 | 15.853 | 19.791 | 11.719 | 1.00 | 38.98 | C |
| ATOM | 970 | CD1 | PHE | A | 123 | 14.771 | 20.646 | 11.733 | 1.00 | 42.53 | C |
| ATOM | 971 | CE1 | PHE | A | 123 | 13.476 | 20.154 | 11.780 | 1.00 | 41.61 | C |
| ATOM | 972 | CZ | PHE | A | 123 | 13.252 | 18.805 | 11.779 | 1.00 | 36.70 | C |
| ATOM | 973 | CD2 | PHE | A | 123 | 15.606 | 18.435 | 11.723 | 1.00 | 36.94 | C |
| ATOM | 974 | CE2 | PHE | A | 123 | 14.315 | 17.947 | 11.737 | 1.00 | 35.81 | C |
| ATOM | 975 | C | PHE | A | 123 | 19.238 | 21.233 | 12.988 | 1.00 | 42.49 | C |
| ATOM | 976 | O | PHE | A | 123 | 19.364 | 22.470 | 12.986 | 1.00 | 43.72 | O |
| ATOM | 977 | N | HIS | A | 124 | 20.268 | 20.406 | 12.945 | 1.00 | 43.02 | N |
| ATOM | 978 | CA | HIS | A | 124 | 21.675 | 20.852 | 12.859 | 1.00 | 42.69 | C |
| ATOM | 979 | CB | HIS | A | 124 | 22.575 | 20.087 | 13.839 | 1.00 | 46.59 | C |
| ATOM | 980 | CG | HIS | A | 124 | 22.274 | 20.325 | 15.278 | 1.00 | 48.99 | C |
| ATOM | 981 | ND1 | HIS | A | 124 | 21.840 | 21.546 | 15.749 | 1.00 | 53.25 | N |
| ATOM | 982 | CE1 | HIS | A | 124 | 21.680 | 21.484 | 17.055 | 1.00 | 50.92 | C |
| ATOM | 983 | NE2 | HIS | A | 124 | 22.012 | 20.249 | 17.452 | 1.00 | 53.60 | N |
| ATOM | 984 | CD2 | HIS | A | 124 | 22.394 | 19.523 | 16.357 | 1.00 | 52.62 | C |
| ATOM | 985 | C | HIS | A | 124 | 22.106 | 20.636 | 11.423 | 1.00 | 39.85 | C |
| ATOM | 986 | O | HIS | A | 124 | 21.907 | 19.534 | 10.925 | 1.00 | 42.55 | O |
| ATOM | 987 | N | PHE | A | 125 | 22.664 | 21.658 | 10.797 | 1.00 | 42.35 | N |
| ATOM | 988 | CA | PHE | A | 125 | 23.350 | 21.567 | 9.492 | 1.00 | 41.35 | C |
| ATOM | 989 | CB | PHE | A | 125 | 22.559 | 22.284 | 8.399 | 1.00 | 43.71 | C |
| ATOM | 990 | CG | PHE | A | 125 | 21.146 | 21.787 | 8.241 | 1.00 | 47.92 | C |
| ATOM | 991 | CD1 | PHE | A | 125 | 20.881 | 20.587 | 7.605 | 1.00 | 47.77 | C |
| ATOM | 992 | CE1 | PHE | A | 125 | 19.576 | 20.142 | 7.461 | 1.00 | 48.75 | C |
| ATOM | 993 | CZ | PHE | A | 125 | 18.532 | 20.883 | 7.951 | 1.00 | 47.20 | C |
| ATOM | 994 | CD2 | PHE | A | 125 | 20.079 | 22.521 | 8.732 | 1.00 | 45.76 | C |
| ATOM | 995 | CE2 | PHE | A | 125 | 18.780 | 22.076 | 8.582 | 1.00 | 45.98 | C |
| ATOM | 996 | C | PHE | A | 125 | 24.723 | 22.192 | 9.666 | 1.00 | 44.44 | C |
| ATOM | 997 | O | PHE | A | 125 | 24.910 | 22.991 | 10.606 | 1.00 | 44.56 | O |
| ATOM | 998 | N | TRP | A | 126 | 25.623 | 21.827 | 8.764 | 1.00 | 42.00 | N |
| ATOM | 999 | CA | TRP | A | 126 | 27.046 | 22.192 | 8.784 | 1.00 | 39.37 | C |
| ATOM | 1000 | CB | TRP | A | 126 | 27.889 | 20.950 | 8.508 | 1.00 | 42.23 | C |
| ATOM | 1001 | CG | TRP | A | 126 | 29.360 | 21.228 | 8.419 | 1.00 | 43.68 | C |
| ATOM | 1002 | CD1 | TRP | A | 126 | 30.109 | 21.399 | 7.289 | 1.00 | 45.37 | C |
| ATOM | 1003 | NE1 | TRP | A | 126 | 31.419 | 21.601 | 7.623 | 1.00 | 47.40 | N |
| ATOM | 1004 | CE2 | TRP | A | 126 | 31.543 | 21.583 | 8.987 | 1.00 | 42.25 | C |
| ATOM | 1005 | CD2 | TRP | A | 126 | 30.262 | 21.374 | 9.525 | 1.00 | 39.01 | C |
| ATOM | 1006 | CE3 | TRP | A | 126 | 30.111 | 21.317 | 10.916 | 1.00 | 45.80 | C |
| ATOM | 1007 | CZ3 | TRP | A | 126 | 31.217 | 21.502 | 11.719 | 1.00 | 44.28 | C |
| ATOM | 1008 | CH2 | TRP | A | 126 | 32.477 | 21.713 | 11.160 | 1.00 | 42.22 | C |
| ATOM | 1009 | CZ2 | TRP | A | 126 | 32.666 | 21.755 | 9.795 | 1.00 | 42.12 | C |
| ATOM | 1010 | C | TRP | A | 126 | 27.273 | 23.252 | 7.718 | 1.00 | 41.21 | C |
| ATOM | 1011 | O | TRP | A | 126 | 27.184 | 22.913 | 6.522 | 1.00 | 42.00 | O |
| ATOM | 1012 | N | ARG | A | 127 | 27.581 | 24.474 | 8.141 | 1.00 | 44.87 | N |
| ATOM | 1013 | CA | ARG | A | 127 | 27.758 | 25.634 | 7.232 | 1.00 | 45.69 | C |
| ATOM | 1014 | CB | ARG | A | 127 | 26.508 | 26.519 | 7.289 | 1.00 | 48.24 | C |
| ATOM | 1015 | CG | ARG | A | 127 | 25.233 | 25.834 | 6.805 | 1.00 | 54.82 | C |
| ATOM | 1016 | CD | ARG | A | 127 | 25.241 | 25.389 | 5.348 | 1.00 | 55.83 | C |
| ATOM | 1017 | NE | ARG | A | 127 | 25.127 | 26.452 | 4.349 | 1.00 | 52.30 | N |
| ATOM | 1018 | CZ | ARG | A | 127 | 25.484 | 26.324 | 3.068 | 1.00 | 59.30 | C |
| ATOM | 1019 | NH1 | ARG | A | 127 | 25.998 | 25.188 | 2.627 | 1.00 | 64.46 | N |
| ATOM | 1020 | NH2 | ARG | A | 127 | 25.329 | 27.325 | 2.215 | 1.00 | 63.81 | N |
| ATOM | 1021 | C | ARG | A | 127 | 29.014 | 26.395 | 7.652 | 1.00 | 42.54 | C |
| ATOM | 1022 | O | ARG | A | 127 | 29.143 | 26.699 | 8.846 | 1.00 | 39.37 | O |
| ATOM | 1023 | N | PHE | A | 128 | 29.913 | 26.658 | 6.705 | 1.00 | 43.55 | N |
| ATOM | 1024 | CA | PHE | A | 128 | 31.154 | 27.439 | 6.932 | 1.00 | 48.56 | C |
| ATOM | 1025 | CB | PHE | A | 128 | 30.834 | 28.938 | 6.866 | 1.00 | 51.33 | C |
| ATOM | 1026 | CG | PHE | A | 128 | 30.061 | 29.313 | 5.625 | 1.00 | 49.58 | C |
| ATOM | 1027 | CD1 | PHE | A | 128 | 30.657 | 29.234 | 4.376 | 1.00 | 47.29 | C |
| ATOM | 1028 | CE1 | PHE | A | 128 | 29.932 | 29.514 | 3.225 | 1.00 | 50.56 | C |
| ATOM | 1029 | CZ | PHE | A | 128 | 28.595 | 29.841 | 3.312 | 1.00 | 49.44 | C |
| ATOM | 1030 | CD2 | PHE | A | 128 | 28.705 | 29.628 | 5.695 | 1.00 | 53.24 | C |
| ATOM | 1031 | CE2 | PHE | A | 128 | 27.978 | 29.890 | 4.543 | 1.00 | 48.70 | C |
| ATOM | 1032 | C | PHE | A | 128 | 31.769 | 26.977 | 8.256 | 1.00 | 52.39 | C |
| ATOM | 1033 | O | PHE | A | 128 | 31.864 | 27.801 | 9.186 | 1.00 | 54.77 | O |
| ATOM | 1034 | N | GLY | A | 129 | 32.082 | 25.673 | 8.342 | 1.00 | 49.98 | N |
| ATOM | 1035 | CA | GLY | A | 129 | 32.942 | 25.057 | 9.369 | 1.00 | 51.54 | C |
| ATOM | 1036 | C | GLY | A | 129 | 32.289 | 24.977 | 10.739 | 1.00 | 52.65 | C |
| ATOM | 1037 | O | GLY | A | 129 | 33.023 | 24.780 | 11.717 | 1.00 | 54.01 | O |
| ATOM | 1038 | N | GLU | A | 130 | 30.961 | 25.043 | 10.815 | 1.00 | 56.42 | N |
| ATOM | 1039 | CA | GLU | A | 130 | 30.224 | 25.152 | 12.101 | 1.00 | 59.51 | C |
| ATOM | 1040 | CB | GLU | A | 130 | 30.001 | 26.643 | 12.351 | 1.00 | 69.62 | C |
| ATOM | 1041 | CG | GLU | A | 130 | 29.665 | 27.003 | 13.787 | 1.00 | 78.68 | C |
| ATOM | 1042 | CD | GLU | A | 130 | 29.352 | 28.479 | 13.966 | 1.00 | 82.34 | C |
| ATOM | 1043 | OE1 | GLU | A | 130 | 28.951 | 29.119 | 12.959 | 1.00 | 74.82 | O |
| ATOM | 1044 | OE2 | GLU | A | 130 | 29.523 | 28.988 | 15.102 | 1.00 | 91.30 | O |
| ATOM | 1045 | O | GLU | A | 130 | 28.890 | 24.379 | 12.050 | 1.00 | 53.26 | O |
| ATOM | 1046 | O | GLU | A | 130 | 28.244 | 24.356 | 10.993 | 1.00 | 48.35 | O |
| ATOM | 1047 | N | TRP | A | 131 | 28.462 | 23.812 | 13.176 | 1.00 | 47.59 | N |
| ATOM | 1048 | CA | TRP | A | 131 | 27.102 | 23.245 | 13.317 | 1.00 | 52.77 | C |
| ATOM | 1049 | CB | TRP | A | 131 | 26.995 | 22.136 | 14.379 | 1.00 | 52.70 | C |
| ATOM | 1050 | CG | TRP | A | 131 | 27.565 | 20.826 | 13.935 | 1.00 | 55.72 | C |
| ATOM | 1051 | CD1 | TRP | A | 131 | 28.706 | 20.232 | 14.390 | 1.00 | 58.20 | C |
| ATOM | 1052 | NE1 | TRP | A | 131 | 28.933 | 19.064 | 13.720 | 1.00 | 63.92 | N |
| ATOM | 1053 | CE2 | TRP | A | 131 | 27.938 | 18.875 | 12.798 | 1.00 | 64.82 | C |
| ATOM | 1054 | CD2 | TRP | A | 131 | 27.051 | 19.968 | 12.902 | 1.00 | 63.99 | C |
| ATOM | 1055 | CE3 | TRP | A | 131 | 25.933 | 20.009 | 12.060 | 1.00 | 59.89 | C |
| ATOM | 1056 | CZ3 | TRP | A | 131 | 25.733 | 18.982 | 11.167 | 1.00 | 54.26 | C |
| ATOM | 1057 | CH2 | TRP | A | 131 | 26.618 | 17.909 | 11.088 | 1.00 | 58.06 | C |
| ATOM | 1058 | CZ2 | TRP | A | 131 | 27.732 | 17.836 | 11.891 | 1.00 | 59.61 | C |
| ATOM | 1059 | C | TRP | A | 131 | 26.157 | 24.414 | 13.589 | 1.00 | 52.97 | C |
| ATOM | 1060 | O | TRP | A | 131 | 26.348 | 25.142 | 14.564 | 1.00 | 59.63 | O |
| ATOM | 1061 | N | VAL | A | 132 | 25.182 | 24.576 | 12.711 | 1.00 | 56.05 | N |
| ATOM | 1062 | CA | VAL | A | 132 | 24.184 | 25.675 | 12.744 | 1.00 | 55.71 | C |
| ATOM | 1063 | CB | VAL | A | 132 | 24.149 | 26.357 | 11.363 | 1.00 | 60.87 | C |
| ATOM | 1064 | CG1 | VAL | A | 132 | 22.790 | 26.942 | 11.029 | 1.00 | 63.69 | C |
| ATOM | 1065 | CG2 | VAL | A | 132 | 25.246 | 27.407 | 11.240 | 1.00 | 58.38 | C |
| ATOM | 1066 | C | VAL | A | 132 | 22.873 | 25.012 | 13.149 | 1.00 | 52.15 | C |
| ATOM | 1067 | O | VAL | A | 132 | 22.634 | 23.886 | 12.707 | 1.00 | 57.06 | O |
| ATOM | 1068 | N | ASP | A | 133 | 22.102 | 25.653 | 14.011 | 1.00 | 48.65 | N |
| ATOM | 1069 | CA | ASP | A | 133 | 20.874 | 25.066 | 14.595 | 1.00 | 52.30 | C |
| ATOM | 1070 | CB | ASP | A | 133 | 20.923 | 25.118 | 16.116 | 1.00 | 50.57 | C |
| ATOM | 1071 | CG | ASP | A | 133 | 19.606 | 24.701 | 16.713 | 1.00 | 56.85 | C |
| ATOM | 1072 | OD1 | ASP | A | 133 | 19.262 | 23.509 | 16.625 | 1.00 | 64.69 | O |
| ATOM | 1073 | OD2 | ASP | A | 133 | 18.910 | 25.582 | 17.189 | 1.00 | 80.43 | O |
| ATOM | 1074 | C | ASP | A | 133 | 19.652 | 25.807 | 14.034 | 1.00 | 53.16 | C |
| ATOM | 1075 | O | ASP | A | 133 | 19.419 | 26.951 | 14.441 | 1.00 | 55.75 | O |
| ATOM | 1076 | N | VAL | A | 134 | 18.912 | 25.173 | 13.123 | 1.00 | 49.04 | N |
| ATOM | 1077 | CA | VAL | A | 134 | 17.800 | 25.812 | 12.359 | 1.00 | 45.32 | C |
| ATOM | 1078 | CB | VAL | A | 134 | 17.816 | 25.402 | 10.881 | 1.00 | 40.80 | C |
| ATOM | 1079 | CG1 | VAL | A | 134 | 16.677 | 26.061 | 10.128 | 1.00 | 41.32 | C |
| ATOM | 1080 | CG2 | VAL | A | 134 | 19.145 | 25.763 | 10.242 | 1.00 | 41.40 | C |
| ATOM | 1081 | C | VAL | A | 134 | 16.477 | 25.460 | 13.026 | 1.00 | 40.23 | C |
| ATOM | 1082 | O | VAL | A | 134 | 16.182 | 24.283 | 13.156 | 1.00 | 48.83 | O |
| ATOM | 1083 | N | VAL | A | 135 | 15.750 | 26.465 | 13.482 | 1.00 | 42.90 | N |
| ATOM | 1084 | CA | VAL | A | 135 | 14.455 | 26.297 | 14.195 | 1.00 | 43.52 | C |
| ATOM | 1085 | CB | VAL | A | 135 | 14.330 | 27.289 | 15.360 | 1.00 | 44.81 | C |
| ATOM | 1086 | CG1 | VAL | A | 135 | 12.948 | 27.259 | 15.994 | 1.00 | 45.86 | C |
| ATOM | 1087 | CG2 | VAL | A | 135 | 15.409 | 27.046 | 16.396 | 1.00 | 45.51 | C |
| ATOM | 1088 | C | VAL | A | 135 | 13.363 | 26.533 | 13.161 | 1.00 | 46.64 | C |
| ATOM | 1089 | O | VAL | A | 135 | 13.579 | 27.426 | 12.291 | 1.00 | 43.21 | O |
| ATOM | 1090 | N | ILE | A | 136 | 12.264 | 25.774 | 13.242 | 1.00 | 42.59 | N |
| ATOM | 1091 | CA | ILE | A | 136 | 11.096 | 25.934 | 12.330 | 1.00 | 41.86 | C |
| ATOM | 1092 | CB | ILE | A | 136 | 11.215 | 25.023 | 11.092 | 1.00 | 44.79 | C |
| ATOM | 1093 | CG1 | ILE | A | 136 | 11.326 | 23.550 | 11.500 | 1.00 | 51.20 | C |
| ATOM | 1094 | CG2 | ILE | A | 136 | 12.351 | 25.455 | 10.172 | 1.00 | 42.31 | C |
| ATOM | 1095 | CD1 | ILE | A | 136 | 11.077 | 22.572 | 10.375 | 1.00 | 51.43 | C |
| ATOM | 1096 | C | ILE | A | 136 | 9.823 | 25.629 | 13.107 | 1.00 | 40.45 | C |
| ATOM | 1097 | O | ILE | A | 136 | 9.904 | 24.924 | 14.099 | 1.00 | 39.18 | O |
| ATOM | 1098 | N | ASP | A | 137 | 8.686 | 26.149 | 12.648 | 1.00 | 44.23 | N |
| ATOM | 1099 | CA | ASP | A | 137 | 7.349 | 25.624 | 13.025 | 1.00 | 42.26 | C |
| ATOM | 1100 | CB | ASP | A | 137 | 6.281 | 26.729 | 13.047 | 1.00 | 43.92 | C |
| ATOM | 1101 | CG | ASP | A | 137 | 5.947 | 27.347 | 11.687 | 1.00 | 45.56 | C |
| ATOM | 1102 | OD1 | ASP | A | 137 | 5.558 | 26.608 | 10.758 | 1.00 | 43.99 | O |
| ATOM | 1103 | OD2 | ASP | A | 137 | 6.095 | 28.562 | 11.546 | 1.00 | 45.89 | O |
| ATOM | 1104 | C | ASP | A | 137 | 7.051 | 24.508 | 12.022 | 1.00 | 41.23 | C |
| ATOM | 1105 | O | ASP | A | 137 | 7.630 | 24.540 | 10.897 | 1.00 | 40.09 | O |
| ATOM | 1106 | N | ASP | A | 138 | 6.149 | 23.602 | 12.376 | 1.00 | 44.15 | N |
| ATOM | 1107 | CA | ASP | A | 138 | 5.869 | 22.376 | 11.578 | 1.00 | 45.24 | C |
| ATOM | 1108 | CB | ASP | A | 138 | 5.594 | 21.183 | 12.495 | 1.00 | 48.30 | C |
| ATOM | 1109 | CG | ASP | A | 138 | 4.448 | 21.397 | 13.459 | 1.00 | 49.14 | C |
| ATOM | 1110 | OD1 | ASP | A | 138 | 3.831 | 22.476 | 13.382 | 1.00 | 48.23 | O |
| ATOM | 1111 | OD2 | ASP | A | 138 | 4.189 | 20.477 | 14.271 | 1.00 | 50.21 | O |
| ATOM | 1112 | C | ASP | A | 138 | 4.705 | 22.569 | 10.593 | 1.00 | 44.16 | C |
| ATOM | 1113 | O | ASP | A | 138 | 4.161 | 21.530 | 10.161 | 1.00 | 41.37 | O |
| ATOM | 1114 | N | ARG | A | 139 | 4.358 | 23.802 | 10.192 | 1.00 | 40.57 | N |
| ATOM | 1115 | CA | ARG | A | 139 | 3.331 | 24.010 | 9.129 | 1.00 | 38.74 | C |
| ATOM | 1116 | CB | ARG | A | 139 | 2.725 | 25.415 | 9.136 | 1.00 | 41.52 | C |
| ATOM | 1117 | CG | ARG | A | 139 | 1.969 | 25.748 | 10.416 | 1.00 | 46.63 | C |
| ATOM | 1118 | CD | ARG | A | 139 | 1.482 | 27.177 | 10.475 | 1.00 | 48.77 | C |
| ATOM | 1119 | NE | ARG | A | 139 | 2.560 | 28.149 | 10.520 | 1.00 | 54.94 | N |
| ATOM | 1120 | CZ | ARG | A | 139 | 2.399 | 29.455 | 10.718 | 1.00 | 58.71 | C |
| ATOM | 1121 | NH1 | ARG | A | 139 | 1.190 | 29.960 | 10.875 | 1.00 | 60.65 | N |
| ATOM | 1122 | NH2 | ARG | A | 139 | 3.450 | 30.251 | 10.772 | 1.00 | 59.21 | N |
| ATOM | 1123 | C | ARG | A | 139 | 3.984 | 23.719 | 7.782 | 1.00 | 38.54 | C |
| ATOM | 1124 | O | ARG | A | 139 | 5.105 | 24.233 | 7.534 | 1.00 | 37.00 | O |
| ATOM | 1125 | N | LEU | A | 140 | 3.316 | 22.919 | 6.950 | 1.00 | 38.70 | N |
| ATOM | 1126 | CA | LEU | A | 140 | 3.854 | 22.489 | 5.644 | 1.00 | 37.51 | C |
| ATOM | 1127 | CB | LEU | A | 140 | 4.122 | 20.987 | 5.700 | 1.00 | 39.53 | C |
| ATOM | 1128 | CG | LEU | A | 140 | 5.222 | 20.577 | 6.667 | 1.00 | 37.63 | C |
| ATOM | 1129 | CD1 | LEU | A | 140 | 5.001 | 19.160 | 7.122 | 1.00 | 38.43 | C |
| ATOM | 1130 | CD2 | LEU | A | 140 | 6.586 | 20.741 | 6.018 | 1.00 | 37.01 | C |
| ATOM | 1131 | C | LEU | A | 140 | 2.874 | 22.814 | 4.540 | 1.00 | 38.30 | C |
| ATOM | 1132 | O | LEU | A | 140 | 1.661 | 22.745 | 4.747 | 1.00 | 38.49 | O |
| ATOM | 1133 | N | PRO | A | 141 | 3.412 | 23.078 | 3.328 | 1.00 | 39.09 | N |
| ATOM | 1134 | CA | PRO | A | 141 | 2.620 | 23.369 | 2.143 | 1.00 | 38.51 | C |
| ATOM | 1135 | CB | PRO | A | 141 | 3.639 | 23.632 | 1.018 | 1.00 | 39.60 | C |
| ATOM | 1136 | CG | PRO | A | 141 | 4.967 | 23.797 | 1.717 | 1.00 | 39.59 | C |
| ATOM | 1137 | CD | PRO | A | 141 | 4.841 | 23.023 | 3.012 | 1.00 | 41.08 | C |
| ATOM | 1138 | C | PRO | A | 141 | 1.816 | 22.141 | 1.731 | 1.00 | 41.26 | C |
| ATOM | 1139 | O | PRO | A | 141 | 2.417 | 21.090 | 1.657 | 1.00 | 48.54 | O |
| ATOM | 1140 | N | THR | A | 142 | 0.541 | 22.339 | 1.399 | 1.00 | 44.55 | N |
| ATOM | 1141 | CA | THR | A | 142 | −0.476 | 21.284 | 1.140 | 1.00 | 41.88 | C |
| ATOM | 1142 | CB | THR | A | 142 | −1.313 | 21.085 | 2.412 | 1.00 | 44.79 | C |
| ATOM | 1143 | OG1 | THR | A | 142 | −0.872 | 19.867 | 3.010 | 1.00 | 47.71 | O |
| ATOM | 1144 | CG2 | THR | A | 142 | −2.810 | 21.119 | 2.176 | 1.00 | 43.29 | C |
| ATOM | 1145 | C | THR | A | 142 | −1.322 | 21.684 | −0.069 | 1.00 | 39.30 | C |
| ATOM | 1146 | O | THR | A | 142 | −1.572 | 22.883 | −0.256 | 1.00 | 41.78 | O |
| ATOM | 1147 | N | VAL | A | 143 | −1.744 | 20.697 | −0.840 | 1.00 | 37.56 | N |
| ATOM | 1148 | CA | VAL | A | 143 | −2.729 | 20.811 | −1.944 | 1.00 | 39.56 | C |
| ATOM | 1149 | CB | VAL | A | 143 | −2.042 | 20.922 | −3.309 | 1.00 | 38.10 | C |
| ATOM | 1150 | CG1 | VAL | A | 143 | −3.056 | 20.791 | −4.435 | 1.00 | 38.30 | C |
| ATOM | 1151 | CG2 | VAL | A | 143 | −1.242 | 22.201 | −3.433 | 1.00 | 39.29 | C |
| ATOM | 1152 | C | VAL | A | 143 | −3.587 | 19.551 | −1.893 | 1.00 | 47.09 | C |
| ATOM | 1153 | O | VAL | A | 143 | −2.988 | 18.440 | −1.979 | 1.00 | 40.57 | O |
| ATOM | 1154 | N | ASN | A | 144 | −4.914 | 19.709 | −1.789 | 1.00 | 49.25 | N |
| ATOM | 1155 | CA | ASN | A | 144 | −5.873 | 18.575 | −1.690 | 1.00 | 50.64 | C |
| ATOM | 1156 | CB | ASN | A | 144 | −5.889 | 17.670 | −2.925 | 1.00 | 50.78 | C |
| ATOM | 1157 | CG | ASN | A | 144 | −6.407 | 18.341 | −4.174 | 1.00 | 60.16 | C |
| ATOM | 1158 | OD1 | ASN | A | 144 | −7.262 | 19.223 | −4.116 | 1.00 | 78.84 | O |
| ATOM | 1159 | ND2 | ASN | A | 144 | −5.903 | 17.912 | −5.318 | 1.00 | 66.22 | N |
| ATOM | 1160 | C | ASN | A | 144 | −5.476 | 17.720 | −0.493 | 1.00 | 45.16 | C |
| ATOM | 1161 | O | ASN | A | 144 | −5.474 | 16.505 | −0.642 | 1.00 | 40.02 | O |
| ATOM | 1162 | N | ASN | A | 145 | −5.073 | 18.366 | 0.601 | 1.00 | 48.82 | N |
| ATOM | 1163 | CA | ASN | A | 145 | −4.689 | 17.752 | 1.902 | 1.00 | 50.35 | C |
| ATOM | 1164 | CB | ASN | A | 145 | −5.865 | 16.995 | 2.508 | 1.00 | 46.80 | C |
| ATOM | 1165 | CG | ASN | A | 145 | −5.773 | 16.952 | 4.017 | 1.00 | 51.19 | C |
| ATOM | 1166 | OD1 | ASN | A | 145 | −5.534 | 17.975 | 4.651 | 1.00 | 53.87 | O |
| ATOM | 1167 | ND2 | ASN | A | 145 | −5.950 | 15.778 | 4.601 | 1.00 | 48.12 | N |
| ATOM | 1168 | C | ASN | A | 145 | −3.455 | 16.832 | 1.785 | 1.00 | 49.21 | C |
| ATOM | 1169 | O | ASN | A | 145 | −3.308 | 15.939 | 2.643 | 1.00 | 49.07 | O |
| ATOM | 1170 | N | GLN | A | 146 | −2.570 | 17.047 | 0.812 | 1.00 | 46.65 | N |
| ATOM | 1171 | CA | GLN | A | 146 | −1.370 | 16.189 | 0.610 | 1.00 | 48.04 | C |
| ATOM | 1172 | CB | GLN | A | 146 | −1.478 | 15.351 | −0.658 | 1.00 | 52.77 | C |
| ATOM | 1173 | CG | GLN | A | 146 | −2.497 | 14.227 | −0.576 | 1.00 | 58.51 | C |
| ATOM | 1174 | CD | GLN | A | 146 | −2.797 | 13.697 | −1.960 | 1.00 | 66.78 | C |
| ATOM | 1175 | OE1 | GLN | A | 146 | −1.917 | 13.576 | −2.818 | 1.00 | 67.21 | O |
| ATOM | 1176 | NE2 | GLN | A | 146 | −4.060 | 13.391 | −2.201 | 1.00 | 73.17 | N |
| ATOM | 1177 | C | GLN | A | 146 | −0.114 | 17.050 | 0.489 | 1.00 | 43.29 | C |
| ATOM | 1178 | O | GLN | A | 146 | −0.170 | 18.115 | −0.155 | 1.00 | 42.05 | O |
| ATOM | 1179 | N | LEU | A | 147 | 0.998 | 16.550 | 1.023 | 1.00 | 40.23 | N |
| ATOM | 1180 | CA | LEU | A | 147 | 2.324 | 17.212 | 0.940 | 1.00 | 33.29 | C |
| ATOM | 1181 | CB | LEU | A | 147 | 3.286 | 16.430 | 1.825 | 1.00 | 28.54 | C |
| ATOM | 1182 | CG | LEU | A | 147 | 3.008 | 16.538 | 3.316 | 1.00 | 26.51 | C |
| ATOM | 1183 | CD1 | LEU | A | 147 | 3.986 | 15.680 | 4.103 | 1.00 | 28.18 | C |
| ATOM | 1184 | CD2 | LEU | A | 147 | 3.104 | 17.983 | 3.771 | 1.00 | 28.32 | C |
| ATOM | 1185 | C | LEU | A | 147 | 2.753 | 17.235 | −0.521 | 1.00 | 30.24 | C |
| ATOM | 1186 | O | LEU | A | 147 | 2.591 | 16.203 | −1.189 | 1.00 | 32.12 | O |
| ATOM | 1187 | N | ILE | A | 148 | 3.216 | 18.375 | −1.025 | 1.00 | 29.68 | N |
| ATOM | 1188 | CA | ILE | A | 148 | 3.619 | 18.492 | −2.462 | 1.00 | 31.75 | C |
| ATOM | 1189 | CB | ILE | A | 148 | 2.983 | 19.735 | −3.106 | 1.00 | 36.05 | C |
| ATOM | 1190 | CG1 | ILE | A | 148 | 3.244 | 20.997 | −2.277 | 1.00 | 39.53 | C |
| ATOM | 1191 | CG2 | ILE | A | 148 | 1.488 | 19.514 | −3.324 | 1.00 | 40.24 | C |
| ATOM | 1192 | CD1 | ILE | A | 148 | 2.966 | 22.285 | −3.026 | 1.00 | 41.96 | C |
| ATOM | 1193 | C | ILE | A | 148 | 5.154 | 18.460 | −2.582 | 1.00 | 30.41 | C |
| ATOM | 1194 | O | ILE | A | 148 | 5.689 | 18.315 | −3.702 | 1.00 | 30.25 | O |
| ATOM | 1195 | N | TYR | A | 149 | 5.865 | 18.559 | −1.477 | 1.00 | 31.63 | N |
| ATOM | 1196 | CA | TYR | A | 149 | 7.344 | 18.433 | −1.474 | 1.00 | 38.50 | C |
| ATOM | 1197 | CB | TYR | A | 149 | 8.004 | 19.601 | −0.727 | 1.00 | 35.61 | C |
| ATOM | 1198 | CG | TYR | A | 149 | 7.750 | 20.952 | −1.342 | 1.00 | 35.19 | C |
| ATOM | 1199 | CD1 | TYR | A | 149 | 8.499 | 21.417 | −2.409 | 1.00 | 36.20 | C |
| ATOM | 1200 | CE1 | TYR | A | 149 | 8.256 | 22.662 | −2.980 | 1.00 | 35.96 | C |
| ATOM | 1201 | CZ | TYR | A | 149 | 7.254 | 23.471 | −2.472 | 1.00 | 34.89 | C |
| ATOM | 1202 | OH | TYR | A | 149 | 6.967 | 24.696 | −3.005 | 1.00 | 37.65 | O |
| ATOM | 1203 | CE2 | TYR | A | 149 | 6.509 | 23.031 | −1.396 | 1.00 | 36.06 | C |
| ATOM | 1204 | CD2 | TYR | A | 149 | 6.747 | 21.780 | −0.853 | 1.00 | 37.65 | C |
| ATOM | 1205 | C | TYR | A | 149 | 7.633 | 17.036 | −0.929 | 1.00 | 37.05 | C |
| ATOM | 1206 | O | TYR | A | 149 | 6.780 | 16.178 | −1.141 | 1.00 | 39.63 | O |
| ATOM | 1207 | N | CYS | A | 150 | 8.746 | 16.818 | −0.233 | 1.00 | 34.69 | N |
| ATOM | 1208 | CA | CYS | A | 150 | 9.201 | 15.452 | 0.120 | 1.00 | 36.92 | C |
| ATOM | 1209 | CB | CYS | A | 150 | 10.702 | 15.487 | 0.360 | 1.00 | 40.38 | C |
| ATOM | 1210 | SG | CYS | A | 150 | 11.473 | 16.142 | −1.148 | 1.00 | 51.68 | S |
| ATOM | 1211 | C | CYS | A | 150 | 8.354 | 14.913 | 1.269 | 1.00 | 31.72 | C |
| ATOM | 1212 | O | CYS | A | 150 | 7.929 | 15.694 | 2.091 | 1.00 | 34.74 | O |
| ATOM | 1213 | N | HIS | A | 151 | 8.077 | 13.617 | 1.275 | 1.00 | 32.12 | N |
| ATOM | 1214 | CA | HIS | A | 151 | 7.217 | 12.953 | 2.289 | 1.00 | 32.92 | C |
| ATOM | 1215 | CB | HIS | A | 151 | 5.734 | 13.288 | 2.047 | 1.00 | 35.78 | C |
| ATOM | 1216 | CG | HIS | A | 151 | 5.230 | 12.916 | 0.692 | 1.00 | 32.19 | C |
| ATOM | 1217 | ND1 | HIS | A | 151 | 5.397 | 13.748 | −0.426 | 1.00 | 34.85 | N |
| ATOM | 1218 | CE1 | HIS | A | 151 | 4.875 | 13.158 | −1.497 | 1.00 | 34.50 | C |
| ATOM | 1219 | NE2 | HIS | A | 151 | 4.369 | 11.966 | −1.116 | 1.00 | 33.11 | N |
| ATOM | 1220 | CD2 | HIS | A | 151 | 4.589 | 11.815 | 0.248 | 1.00 | 33.93 | C |
| ATOM | 1221 | C | HIS | A | 151 | 7.448 | 11.450 | 2.232 | 1.00 | 32.48 | C |
| ATOM | 1222 | O | HIS | A | 151 | 8.128 | 10.996 | 1.322 | 1.00 | 33.55 | O |
| ATOM | 1223 | N | SER | A | 152 | 6.840 | 10.709 | 3.144 | 1.00 | 36.92 | N |
| ATOM | 1224 | CA | SER | A | 152 | 6.879 | 9.227 | 3.164 | 1.00 | 39.11 | C |
| ATOM | 1225 | CB | SER | A | 152 | 7.097 | 8.735 | 4.547 | 1.00 | 44.10 | C |
| ATOM | 1226 | OG | SER | A | 152 | 8.096 | 9.519 | 5.206 | 1.00 | 53.92 | O |
| ATOM | 1227 | C | SER | A | 152 | 5.580 | 8.655 | 2.604 | 1.00 | 39.78 | C |
| ATOM | 1228 | O | SER | A | 152 | 4.606 | 9.401 | 2.465 | 1.00 | 37.55 | O |
| ATOM | 1229 | N | ASN | A | 153 | 5.621 | 7.367 | 2.296 | 1.00 | 43.12 | N |
| ATOM | 1230 | CA | ASN | A | 153 | 4.479 | 6.462 | 2.041 | 1.00 | 46.53 | C |
| ATOM | 1231 | CB | ASN | A | 153 | 4.942 | 5.011 | 2.057 | 1.00 | 57.90 | C |
| ATOM | 1232 | CG | ASN | A | 153 | 4.762 | 4.333 | 0.729 | 1.00 | 67.31 | C |
| ATOM | 1233 | OD1 | ASN | A | 153 | 4.804 | 4.989 | −0.309 | 1.00 | 80.58 | O |
| ATOM | 1234 | ND2 | ASN | A | 153 | 4.566 | 3.026 | 0.770 | 1.00 | 72.05 | N |
| ATOM | 1235 | C | ASN | A | 153 | 3.443 | 6.553 | 3.151 | 1.00 | 44.19 | C |
| ATOM | 1236 | O | ASN | A | 153 | 2.246 | 6.459 | 2.829 | 1.00 | 47.16 | O |
| ATOM | 1237 | N | SER | A | 154 | 3.902 | 6.590 | 4.406 | 1.00 | 42.16 | N |
| ATOM | 1238 | CA | SER | A | 154 | 3.047 | 6.805 | 5.603 | 1.00 | 41.80 | C |
| ATOM | 1239 | CB | SER | A | 154 | 3.755 | 6.508 | 6.901 | 1.00 | 41.80 | C |
| ATOM | 1240 | OG | SER | A | 154 | 3.799 | 5.109 | 7.150 | 1.00 | 49.43 | O |
| ATOM | 1241 | C | SER | A | 154 | 2.585 | 8.252 | 5.572 | 1.00 | 41.05 | C |
| ATOM | 1242 | O | SER | A | 154 | 3.438 | 9.109 | 5.732 | 1.00 | 39.32 | O |
| ATOM | 1243 | N | ARG | A | 155 | 1.297 | 8.527 | 5.355 | 1.00 | 44.31 | N |
| ATOM | 1244 | CA | ARG | A | 155 | 0.839 | 9.937 | 5.294 | 1.00 | 45.33 | C |
| ATOM | 1245 | CB | ARG | A | 155 | −0.504 | 10.079 | 4.574 | 1.00 | 48.30 | C |
| ATOM | 1246 | CG | ARG | A | 155 | −1.719 | 9.693 | 5.394 | 1.00 | 51.85 | C |
| ATOM | 1247 | CD | ARG | A | 155 | −2.912 | 9.703 | 4.458 | 1.00 | 54.77 | C |
| ATOM | 1248 | NE | ARG | A | 155 | −4.160 | 9.307 | 5.086 | 1.00 | 52.95 | N |
| ATOM | 1249 | CZ | ARG | A | 155 | −5.334 | 9.340 | 4.473 | 1.00 | 53.20 | C |
| ATOM | 125C | NH1 | ARG | A | 155 | −5.418 | 9.753 | 3.221 | 1.00 | 52.79 | N |
| ATOM | 1251 | NH2 | ARG | A | 155 | −6.428 | 8.989 | 5.123 | 1.00 | 58.93 | N |
| ATOM | 1252 | C | ARG | A | 155 | 0.845 | 10.558 | 6.697 | 1.00 | 42.15 | C |
| ATOM | 1253 | O | ARG | A | 155 | 0.585 | 11.750 | 6.790 | 1.00 | 47.82 | O |
| ATOM | 1254 | N | ASN | A | 156 | 1.201 | 9.819 | 7.745 | 1.00 | 41.03 | N |
| ATOM | 1255 | CA | ASN | A | 156 | 1.389 | 10.430 | 9.087 | 1.00 | 41.27 | C |
| ATOM | 1256 | CB | ASN | A | 156 | 0.511 | 9.766 | 10.145 | 1.00 | 42.44 | C |
| ATOM | 1257 | CG | ASN | A | 156 | 0.854 | 8.314 | 10.383 | 1.00 | 44.85 | C |
| ATOM | 1258 | OD1 | ASN | A | 156 | 1.329 | 7.603 | 9.501 | 1.00 | 48.21 | O |
| ATOM | 1259 | ND2 | ASN | A | 156 | 0.609 | 7.860 | 11.591 | 1.00 | 51.99 | N |
| ATOM | 1260 | C | ASN | A | 156 | 2.873 | 10.429 | 9.480 | 1.00 | 36.33 | C |
| ATOM | 1261 | O | ASN | A | 156 | 3.126 | 10.597 | 10.693 | 1.00 | 33.49 | O |
| ATOM | 1262 | N | GLU | A | 157 | 3.796 | 10.300 | 8.512 | 1.00 | 34.42 | N |
| ATOM | 1263 | CA | GLU | A | 157 | 5.259 | 10.401 | 8.778 | 1.00 | 40.59 | C |
| ATOM | 1264 | CB | GLU | A | 157 | 6.055 | 9.224 | 8.200 | 1.00 | 40.47 | C |
| ATOM | 1265 | CG | GLU | A | 157 | 7.515 | 9.241 | 8.650 | 1.00 | 41.13 | C |
| ATOM | 1266 | CD | GLU | A | 157 | 8.400 | 8.043 | 8.335 | 1.00 | 42.95 | C |
| ATOM | 1267 | OE1 | GLU | A | 157 | 8.651 | 7.237 | 9.257 | 1.00 | 51.10 | O |
| ATOM | 1268 | OE2 | GLU | A | 157 | 8.902 | 7.955 | 7.192 | 1.00 | 46.41 | O |
| ATOM | 1269 | C | GLU | A | 157 | 5.798 | 11.745 | 8.270 | 1.00 | 36.41 | C |
| ATOM | 1270 | O | GLU | A | 157 | 5.622 | 12.070 | 7.083 | 1.00 | 36.94 | O |
| ATOM | 1271 | N | PHE | A | 158 | 6.500 | 12.469 | 9.135 | 1.00 | 38.15 | N |
| ATOM | 1272 | CA | PHE | A | 158 | 6.873 | 13.890 | 8.906 | 1.00 | 39.22 | C |
| ATOM | 1273 | CB | PHE | A | 158 | 6.109 | 14.767 | 9.901 | 1.00 | 40.34 | C |
| ATOM | 1274 | CG | PHE | A | 158 | 4.644 | 14.770 | 9.560 | 1.00 | 38.23 | C |
| ATOM | 1275 | CD1 | PHE | A | 158 | 4.165 | 15.583 | 8.550 | 1.00 | 37.00 | C |
| ATOM | 1276 | CE1 | PHE | A | 158 | 2.837 | 15.530 | 8.172 | 1.00 | 38.06 | C |
| ATOM | 1277 | CZ | PHE | A | 158 | 1.985 | 14.647 | 8.781 | 1.00 | 37.39 | C |
| ATOM | 1278 | CD2 | PHE | A | 158 | 3.786 | 13.856 | 10.137 | 1.00 | 40.41 | C |
| ATOM | 1279 | CE2 | PHE | A | 158 | 2.456 | 13.807 | 9.760 | 1.00 | 40.05 | C |
| ATOM | 1280 | C | PHE | A | 158 | 8.376 | 14.168 | 8.946 | 1.00 | 37.76 | C |
| ATOM | 1281 | O | PHE | A | 158 | 8.673 | 15.307 | 8.597 | 1.00 | 39.59 | O |
| ATOM | 1282 | N | TRP | A | 159 | 9.279 | 13.241 | 9.310 | 1.00 | 37.11 | N |
| ATOM | 1283 | CA | TRP | A | 159 | 10.701 | 13.640 | 9.550 | 1.00 | 34.55 | C |
| ATOM | 1284 | CB | TRP | A | 159 | 11.572 | 12.480 | 10.037 | 1.00 | 37.99 | C |
| ATOM | 1285 | CG | TRP | A | 159 | 11.909 | 11.410 | 9.046 | 1.00 | 37.13 | C |
| ATOM | 1286 | CD1 | TRP | A | 159 | 11.240 | 10.240 | 8.833 | 1.00 | 36.47 | C |
| ATOM | 1287 | NE1 | TRP | A | 159 | 11.879 | 9.495 | 7.883 | 1.00 | 38.25 | N |
| ATOM | 1288 | CE2 | TRP | A | 159 | 13.000 | 10.163 | 7.466 | 1.00 | 38.28 | C |
| ATOM | 1289 | CD2 | TRP | A | 159 | 13.060 | 11.376 | 8.185 | 1.00 | 39.21 | C |
| ATOM | 1290 | CE3 | TRP | A | 159 | 14.131 | 12.248 | 7.951 | 1.00 | 37.91 | C |
| ATOM | 1291 | CZ3 | TRP | A | 159 | 15.080 | 11.897 | 7.019 | 1.00 | 37.59 | C |
| ATOM | 1292 | CH2 | TRP | A | 159 | 14.993 | 10.694 | 6.310 | 1.00 | 38.23 | C |
| ATOM | 1293 | CZ2 | TRP | A | 159 | 13.957 | 9.816 | 6.511 | 1.00 | 38.08 | C |
| ATOM | 1294 | C | TRP | A | 159 | 11.287 | 14.268 | 8.292 | 1.00 | 35.84 | C |
| ATOM | 1295 | O | TRP | A | 159 | 11.924 | 15.342 | 8.381 | 1.00 | 37.11 | O |
| ATOM | 1296 | N | CYS | A | 160 | 11.082 | 13.620 | 7.156 | 1.00 | 33.27 | N |
| ATOM | 1297 | CA | CYS | A | 160 | 11.696 | 14.029 | 5.875 | 1.00 | 36.21 | C |
| ATOM | 1298 | CB | CYS | A | 160 | 11.506 | 12.952 | 4.813 | 1.00 | 39.65 | C |
| ATOM | 1299 | SG | CYS | A | 160 | 12.187 | 13.435 | 3.213 | 1.00 | 52.44 | S |
| ATOM | 1300 | C | CYS | A | 160 | 11.078 | 15.361 | 5.454 | 1.00 | 33.36 | C |
| ATOM | 1301 | O | CYS | A | 160 | 11.767 | 16.183 | 4.903 | 1.00 | 38.22 | O |
| ATOM | 1302 | N | ALA | A | 161 | 9.795 | 15.564 | 5.701 | 1.00 | 38.04 | N |
| ATOM | 1303 | CA | ALA | A | 161 | 9.071 | 16.791 | 5.305 | 1.00 | 34.59 | C |
| ATOM | 1304 | CB | ALA | A | 161 | 7.591 | 16.615 | 5.535 | 1.00 | 33.44 | C |
| ATOM | 1305 | C | ALA | A | 161 | 9.644 | 17.964 | 6.106 | 1.00 | 32.11 | C |
| ATOM | 1306 | O | ALA | A | 161 | 9.850 | 19.035 | 5.525 | 1.00 | 31.02 | O |
| ATOM | 1307 | N | LEU | A | 162 | 9.895 | 17.748 | 7.398 | 1.00 | 31.30 | N |
| ATOM | 1308 | CA | LEU | A | 162 | 10.386 | 18.809 | 8.317 | 1.00 | 34.30 | C |
| ATOM | 1309 | CB | LEU | A | 162 | 10.143 | 18.371 | 9.759 | 1.00 | 33.35 | C |
| ATOM | 1310 | CG | LEU | A | 162 | 8.673 | 18.241 | 10.143 | 1.00 | 35.59 | C |
| ATOM | 1311 | CD1 | LEU | A | 162 | 8.511 | 17.731 | 11.571 | 1.00 | 36.98 | C |
| ATOM | 1312 | CD2 | LEU | A | 162 | 7.972 | 19.566 | 9.953 | 1.00 | 37.02 | C |
| ATOM | 1313 | C | LEU | A | 162 | 11.869 | 19.108 | 8.064 | 1.00 | 37.94 | C |
| ATOM | 1314 | O | LEU | A | 162 | 12.230 | 20.268 | 8.138 | 1.00 | 43.70 | O |
| ATOM | 1315 | N | VAL | A | 163 | 12.711 | 18.104 | 7.815 | 1.00 | 38.53 | N |
| ATOM | 1316 | CA | VAL | A | 163 | 14.142 | 18.348 | 7.498 | 1.00 | 36.50 | C |
| ATOM | 1317 | CB | VAL | A | 163 | 14.938 | 17.041 | 7.324 | 1.00 | 38.75 | C |
| ATOM | 1318 | CG1 | VAL | A | 163 | 16.358 | 17.348 | 6.878 | 1.00 | 41.39 | C |
| ATOM | 1319 | CG2 | VAL | A | 163 | 14.969 | 16.212 | 8.604 | 1.00 | 39.41 | C |
| ATOM | 1320 | C | VAL | A | 163 | 14.183 | 19.214 | 6.237 | 1.00 | 35.14 | C |
| ATOM | 1321 | O | VAL | A | 163 | 15.085 | 20.109 | 6.116 | 1.00 | 30.81 | O |
| ATOM | 1322 | N | GLU | A | 164 | 13.255 | 18.972 | 5.312 | 1.00 | 31.64 | N |
| ATOM | 1323 | CA | GLU | A | 164 | 13.270 | 19.712 | 4.028 | 1.00 | 32.03 | C |
| ATOM | 1324 | CB | GLU | A | 164 | 12.303 | 19.094 | 3.035 | 1.00 | 32.89 | C |
| ATOM | 1325 | CG | GLU | A | 164 | 12.458 | 19.635 | 1.636 | 1.00 | 34.12 | C |
| ATOM | 1326 | CD | GLU | A | 164 | 11.331 | 19.245 | 0.692 | 1.00 | 32.95 | C |
| ATOM | 1327 | OE1 | GLU | A | 164 | 11.559 | 19.269 | −0.547 | 1.00 | 38.73 | O |
| ATOM | 1328 | OE2 | GLU | A | 164 | 10.244 | 18.896 | 1.188 | 1.00 | 30.64 | O |
| ATOM | 1329 | C | GLU | A | 164 | 12.902 | 21.169 | 4.313 | 1.00 | 33.51 | C |
| ATOM | 1330 | O | GLU | A | 164 | 13.569 | 22.063 | 3.785 | 1.00 | 34.85 | O |
| ATOM | 1331 | N | LYS | A | 165 | 11.887 | 21.394 | 5.140 | 1.00 | 33.08 | N |
| ATOM | 1332 | CA | LYS | A | 165 | 11.443 | 22.760 | 5.477 | 1.00 | 35.68 | C |
| ATOM | 1333 | CB | LYS | A | 165 | 10.262 | 22.751 | 6.445 | 1.00 | 36.04 | C |
| ATOM | 1334 | CG | LYS | A | 165 | 9.461 | 24.045 | 6.391 | 1.00 | 37.81 | C |
| ATOM | 1335 | CD | LYS | A | 165 | 8.572 | 24.263 | 7.584 | 1.00 | 35.28 | C |
| ATOM | 1336 | CE | LYS | A | 165 | 8.140 | 25.698 | 7.703 | 1.00 | 34.02 | C |
| ATOM | 1337 | NZ | LYS | A | 165 | 6.911 | 25.795 | 8.521 | 1.00 | 38.23 | N |
| ATOM | 1338 | C | LYS | A | 165 | 12.636 | 23.525 | 6.057 | 1.00 | 32.31 | C |
| ATOM | 1339 | O | LYS | A | 165 | 12.925 | 24.597 | 5.550 | 1.00 | 34.60 | O |
| ATOM | 1340 | N | ALA | A | 166 | 13.308 | 22.958 | 7.057 | 1.00 | 34.78 | N |
| ATOM | 1341 | CA | ALA | A | 166 | 14.521 | 23.515 | 7.711 | 1.00 | 35.76 | C |
| ATOM | 1342 | CB | ALA | A | 166 | 15.048 | 22.567 | 8.751 | 1.00 | 36.89 | C |
| ATOM | 1343 | C | ALA | A | 166 | 15.586 | 23.790 | 6.654 | 1.00 | 34.49 | C |
| ATOM | 1344 | O | ALA | A | 166 | 16.137 | 24.881 | 6.617 | 1.00 | 40.37 | O |
| ATOM | 1345 | N | TYR | A | 167 | 15.815 | 22.841 | 5.769 | 1.00 | 33.51 | N |
| ATOM | 1346 | CA | TYR | A | 167 | 16.797 | 23.022 | 4.690 | 1.00 | 33.16 | C |
| ATOM | 1347 | CB | TYR | A | 167 | 17.053 | 21.691 | 4.002 | 1.00 | 34.06 | C |
| ATOM | 1348 | CG | TYR | A | 167 | 18.418 | 21.591 | 3.372 | 1.00 | 32.94 | C |
| ATOM | 1349 | CD1 | TYR | A | 167 | 19.574 | 21.862 | 4.087 | 1.00 | 37.23 | C |
| ATOM | 1350 | CE1 | TYR | A | 167 | 20.825 | 21.768 | 3.493 | 1.00 | 38.08 | C |
| ATOM | 1351 | CZ | TYR | A | 167 | 20.920 | 21.388 | 2.169 | 1.00 | 33.37 | C |
| ATOM | 1352 | OH | TYR | A | 167 | 22.094 | 21.297 | 1.503 | 1.00 | 30.18 | O |
| ATOM | 1353 | CE2 | TYR | A | 167 | 19.775 | 21.136 | 1.452 | 1.00 | 35.41 | C |
| ATOM | 1354 | CD2 | TYR | A | 167 | 18.544 | 21.225 | 2.060 | 1.00 | 31.27 | C |
| ATOM | 1355 | C | TYR | A | 167 | 16.320 | 24.123 | 3.749 | 1.00 | 35.89 | C |
| ATOM | 1356 | O | TYR | A | 167 | 17.160 | 24.853 | 3.150 | 1.00 | 39.38 | O |
| ATOM | 1357 | N | ALA | A | 168 | 15.008 | 24.273 | 3.599 | 1.00 | 36.27 | N |
| ATOM | 1358 | CA | ALA | A | 168 | 14.460 | 25.302 | 2.687 | 1.00 | 31.07 | C |
| ATOM | 1359 | CB | ALA | A | 168 | 13.012 | 25.041 | 2.407 | 1.00 | 31.47 | C |
| ATOM | 1360 | C | ALA | A | 168 | 14.709 | 26.675 | 3.318 | 1.00 | 29.80 | C |
| ATOM | 1361 | O | ALA | A | 168 | 15.144 | 27.554 | 2.597 | 1.00 | 30.74 | O |
| ATOM | 1362 | N | LYS | A | 169 | 14.469 | 26.820 | 4.622 | 1.00 | 28.46 | N |
| ATOM | 1363 | CA | LYS | A | 169 | 14.719 | 28.060 | 5.391 | 1.00 | 32.42 | C |
| ATOM | 1364 | CB | LYS | A | 169 | 14.433 | 27.833 | 6.878 | 1.00 | 32.95 | C |
| ATOM | 1365 | CG | LYS | A | 169 | 14.522 | 29.100 | 7.727 | 1.00 | 35.83 | C |
| ATOM | 1366 | CD | LYS | A | 169 | 14.088 | 28.862 | 9.166 | 1.00 | 37.56 | C |
| ATOM | 1367 | CE | LYS | A | 169 | 14.123 | 30.090 | 10.043 | 1.00 | 36.03 | C |
| ATOM | 1368 | NZ | LYS | A | 169 | 13.675 | 29.748 | 11.417 | 1.00 | 39.53 | N |
| ATOM | 1369 | C | LYS | A | 169 | 16.175 | 28.512 | 5.201 | 1.00 | 37.38 | C |
| ATOM | 1370 | O | LYS | A | 169 | 16.395 | 29.705 | 4.922 | 1.00 | 41.06 | O |
| ATOM | 1371 | N | LEU | A | 170 | 17.127 | 27.586 | 5.359 | 1.00 | 39.20 | N |
| ATOM | 1372 | CA | LEU | A | 170 | 18.577 | 27.803 | 5.119 | 1.00 | 40.72 | C |
| ATOM | 1373 | CB | LEU | A | 170 | 19.293 | 26.477 | 5.382 | 1.00 | 44.64 | C |
| ATOM | 1374 | CG | LEU | A | 170 | 20.797 | 26.542 | 5.607 | 1.00 | 48.13 | C |
| ATOM | 1375 | CD1 | LEU | A | 170 | 21.146 | 27.555 | 6.684 | 1.00 | 52.56 | C |
| ATOM | 1376 | CD2 | LEU | A | 170 | 21.326 | 25.160 | 5.978 | 1.00 | 49.48 | C |
| ATOM | 1377 | C | LEU | A | 170 | 18.804 | 28.292 | 3.685 | 1.00 | 38.94 | C |
| ATOM | 1378 | O | LEU | A | 170 | 19.614 | 29.194 | 3.483 | 1.00 | 41.87 | O |
| ATOM | 1379 | N | ALA | A | 171 | 18.137 | 27.716 | 2.696 | 1.00 | 35.09 | N |
| ATOM | 1380 | CA | ALA | A | 171 | 18.371 | 28.129 | 1.295 | 1.00 | 37.99 | C |
| ATOM | 1381 | CB | ALA | A | 171 | 17.973 | 27.017 | 0.354 | 1.00 | 37.08 | C |
| ATOM | 1382 | C | ALA | A | 171 | 17.628 | 29.452 | 1.013 | 1.00 | 38.47 | C |
| ATOM | 1383 | O | ALA | A | 171 | 17.983 | 30.133 | 0.008 | 1.00 | 38.03 | O |
| ATOM | 1384 | N | GLY | A | 172 | 16.658 | 29.805 | 1.868 | 1.00 | 37.10 | N |
| ATOM | 1385 | CA | GLY | A | 172 | 15.859 | 31.046 | 1.792 | 1.00 | 42.53 | C |
| ATOM | 1386 | C | GLY | A | 172 | 14.406 | 30.809 | 1.394 | 1.00 | 42.99 | C |
| ATOM | 1387 | O | GLY | A | 172 | 13.547 | 31.530 | 1.917 | 1.00 | 46.83 | O |
| ATOM | 1388 | N | CYS | A | 173 | 14.137 | 29.857 | 0.487 | 1.00 | 45.54 | N |
| ATOM | 1389 | CA | CYS | A | 173 | 12.768 | 29.350 | 0.161 | 1.00 | 39.89 | C |
| ATOM | 1390 | CB | CYS | A | 173 | 12.026 | 30.348 | −0.713 | 1.00 | 33.60 | C |
| ATOM | 1391 | SG | CYS | A | 173 | 12.779 | 30.489 | −2.348 | 1.00 | 36.05 | S |
| ATOM | 1392 | C | CYS | A | 173 | 12.836 | 28.004 | −0.575 | 1.00 | 38.25 | C |
| ATOM | 1393 | O | CYS | A | 173 | 13.898 | 27.673 | −1.139 | 1.00 | 42.73 | O |
| ATOM | 1394 | N | TYR | A | 174 | 11.723 | 27.274 | −0.634 | 1.00 | 35.42 | N |
| ATOM | 1395 | CA | TYR | A | 174 | 11.661 | 25.925 | −1.252 | 1.00 | 31.09 | C |
| ATOM | 1396 | CB | TYR | A | 174 | 10.239 | 25.368 | −1.246 | 1.00 | 29.75 | C |
| ATOM | 1397 | CG | TYR | A | 174 | 9.849 | 24.675 | 0.031 | 1.00 | 26.34 | C |
| ATOM | 1398 | CD1 | TYR | A | 174 | 9.475 | 25.394 | 1.140 | 1.00 | 25.65 | C |
| ATOM | 1399 | CE1 | TYR | A | 174 | 9.081 | 24.768 | 2.310 | 1.00 | 26.88 | C |
| ATOM | 1400 | CZ | TYR | A | 174 | 9.065 | 23.389 | 2.389 | 1.00 | 27.53 | C |
| ATOM | 1401 | OH | TYR | A | 174 | 8.704 | 22.763 | 3.560 | 1.00 | 35.06 | O |
| ATOM | 1402 | CE2 | TYR | A | 174 | 9.484 | 22.655 | 1.302 | 1.00 | 25.97 | C |
| ATOM | 1403 | CD2 | TYR | A | 174 | 9.859 | 23.297 | 0.134 | 1.00 | 26.33 | C |
| ATOM | 1404 | C | TYR | A | 174 | 12.201 | 25.982 | −2.675 | 1.00 | 32.90 | C |
| ATOM | 1405 | O | TYR | A | 174 | 12.900 | 25.067 | −3.086 | 1.00 | 34.33 | O |
| ATOM | 1406 | N | GLN | A | 175 | 11.913 | 27.040 | −3.419 | 1.00 | 36.98 | N |
| ATOM | 1407 | CA | GLN | A | 175 | 12.294 | 27.077 | −4.850 | 1.00 | 37.91 | C |
| ATOM | 1408 | CB | GLN | A | 175 | 11.590 | 28.233 | −5.560 | 1.00 | 39.45 | C |
| ATOM | 1409 | CG | GLN | A | 175 | 11.809 | 28.203 | −7.068 | 1.00 | 41.08 | C |
| ATOM | 1410 | CD | GLN | A | 175 | 11.211 | 29.369 | −7.815 | 1.00 | 41.66 | C |
| ATOM | 1411 | OE1 | GLN | A | 175 | 10.427 | 30.163 | −7.292 | 1.00 | 44.93 | O |
| ATOM | 1412 | NE2 | GLN | A | 175 | 11.612 | 29.488 | −9.064 | 1.00 | 42.48 | N |
| ATOM | 1413 | C | GLN | A | 175 | 13.822 | 27.142 | −4.976 | 1.00 | 36.10 | C |
| ATOM | 1414 | O | GLN | A | 175 | 14.332 | 26.855 | −6.059 | 1.00 | 39.22 | O |
| ATOM | 1415 | N | ALA | A | 176 | 14.536 | 27.546 | −3.929 | 1.00 | 38.58 | N |
| ATOM | 1416 | CA | ALA | A | 176 | 16.023 | 27.604 | −3.933 | 1.00 | 42.39 | C |
| ATOM | 1417 | CB | ALA | A | 176 | 16.540 | 28.375 | −2.731 | 1.00 | 38.31 | C |
| ATOM | 1418 | C | ALA | A | 176 | 16.576 | 26.166 | −3.991 | 1.00 | 42.31 | C |
| ATOM | 1419 | O | ALA | A | 176 | 17.606 | 25.952 | −4.649 | 1.00 | 40.15 | O |
| ATOM | 1420 | N | LEU | A | 177 | 15.882 | 25.194 | −3.386 | 1.00 | 38.21 | N |
| ATOM | 1421 | CA | LEU | A | 177 | 16.326 | 23.777 | −3.437 | 1.00 | 41.30 | C |
| ATOM | 1422 | CB | LEU | A | 177 | 15.426 | 22.892 | −2.577 | 1.00 | 41.01 | C |
| ATOM | 1423 | CG | LEU | A | 177 | 15.370 | 23.253 | −1.095 | 1.00 | 42.53 | C |
| ATOM | 1424 | CD1 | LEU | A | 177 | 14.547 | 22.209 | −0.330 | 1.00 | 46.22 | C |
| ATOM | 1425 | CD2 | LEU | A | 177 | 16.768 | 23.386 | −0.503 | 1.00 | 39.00 | C |
| ATOM | 1426 | C | LEU | A | 177 | 16.379 | 23.263 | −4.880 | 1.00 | 42.02 | C |
| ATOM | 1427 | O | LEU | A | 177 | 17.138 | 22.322 | −5.103 | 1.00 | 47.66 | O |
| ATOM | 1428 | N | ASP | A | 178 | 15.679 | 23.860 | −5.843 | 1.00 | 39.56 | N |
| ATOM | 1429 | CA | ASP | A | 178 | 15.697 | 23.358 | −7.245 | 1.00 | 40.00 | C |
| ATOM | 1430 | CB | ASP | A | 178 | 14.710 | 24.105 | −8.146 | 1.00 | 42.41 | C |
| ATOM | 1431 | CG | ASP | A | 178 | 13.243 | 23.998 | −7.732 | 1.00 | 50.63 | C |
| ATOM | 1432 | OD1 | ASP | A | 178 | 12.945 | 23.348 | −6.676 | 1.00 | 51.75 | O |
| ATOM | 1433 | OD2 | ASP | A | 178 | 12.391 | 24.593 | −8.454 | 1.00 | 53.29 | O |
| ATOM | 1434 | C | ASP | A | 178 | 17.107 | 23.456 | −7.839 | 1.00 | 40.16 | C |
| ATOM | 1435 | O | ASP | A | 178 | 17.358 | 22.763 | −8.837 | 1.00 | 43.12 | O |
| ATOM | 1436 | N | GLY | A | 179 | 17.984 | 24.311 | −7.305 | 1.00 | 40.20 | N |
| ATOM | 1437 | CA | GLY | A | 179 | 19.365 | 24.454 | −7.811 | 1.00 | 43.40 | C |
| ATOM | 1438 | C | GLY | A | 179 | 20.401 | 23.678 | −6.996 | 1.00 | 44.81 | C |
| ATOM | 1439 | O | GLY | A | 179 | 21.603 | 23.858 | −7.262 | 1.00 | 46.39 | O |
| ATOM | 1440 | N | GLY | A | 180 | 19.971 | 22.867 | −6.026 | 1.00 | 44.32 | N |
| ATOM | 1441 | CA | GLY | A | 180 | 20.838 | 22.120 | −5.093 | 1.00 | 43.21 | C |
| ATOM | 1442 | C | GLY | A | 180 | 21.265 | 20.767 | −5.655 | 1.00 | 45.65 | C |
| ATOM | 1443 | O | GLY | A | 180 | 20.901 | 20.417 | −6.791 | 1.00 | 42.59 | O |
| ATOM | 1444 | N | ASN | A | 181 | 22.050 | 20.011 | −4.895 | 1.00 | 45.82 | N |
| ATOM | 1445 | CA | ASN | A | 181 | 22.535 | 18.685 | −5.348 | 1.00 | 39.45 | C |
| ATOM | 1446 | CB | ASN | A | 181 | 23.757 | 18.814 | −6.244 | 1.00 | 40.89 | C |
| ATOM | 1447 | CG | ASN | A | 181 | 24.921 | 19.462 | −5.527 | 1.00 | 45.35 | C |
| ATOM | 1448 | OD1 | ASN | A | 181 | 25.537 | 18.871 | −4.631 | 1.00 | 42.78 | O |
| ATOM | 1449 | ND2 | ASN | A | 181 | 25.208 | 20.697 | −5.903 | 1.00 | 48.56 | N |
| ATOM | 1450 | C | ASN | A | 181 | 22.802 | 17.822 | −4.119 | 1.00 | 36.19 | C |
| ATOM | 1451 | O | ASN | A | 181 | 22.929 | 18.366 | −2.995 | 1.00 | 35.88 | O |
| ATOM | 1452 | N | THR | A | 182 | 22.835 | 16.513 | −4.323 | 1.00 | 34.08 | N |
| ATOM | 1453 | CA | THR | A | 182 | 23.025 | 15.505 | −3.249 | 1.00 | 36.74 | C |
| ATOM | 1454 | CB | THR | A | 182 | 22.784 | 14.108 | −3.833 | 1.00 | 37.55 | C |
| ATOM | 1455 | OG1 | THR | A | 182 | 21.456 | 14.120 | −4.355 | 1.00 | 32.92 | O |
| ATOM | 1456 | CG2 | THR | A | 182 | 22.961 | 13.008 | −2.812 | 1.00 | 39.24 | C |
| ATOM | 1457 | C | THR | A | 182 | 24.416 | 15.666 | −2.611 | 1.00 | 36.26 | C |
| ATOM | 1458 | O | THR | A | 182 | 24.527 | 15.623 | −1.371 | 1.00 | 36.12 | O |
| ATOM | 1459 | N | ALA | A | 183 | 25.459 | 15.821 | −3.421 | 1.00 | 39.80 | N |
| ATOM | 1460 | CA | ALA | A | 183 | 26.848 | 16.007 | −2.943 | 1.00 | 41.72 | C |
| ATOM | 1461 | CB | ALA | A | 183 | 27.743 | 16.402 | −4.091 | 1.00 | 44.04 | C |
| ATOM | 1462 | C | ALA | A | 183 | 26.863 | 17.070 | −1.841 | 1.00 | 42.70 | C |
| ATOM | 1463 | O | ALA | A | 183 | 27.339 | 16.770 | −0.728 | 1.00 | 44.58 | O |
| ATOM | 1464 | N | ASP | A | 184 | 26.350 | 18.270 | −2.133 | 1.00 | 41.65 | N |
| ATOM | 1465 | CA | ASP | A | 184 | 26.427 | 19.415 | −1.196 | 1.00 | 40.88 | C |
| ATOM | 1466 | CB | ASP | A | 184 | 26.121 | 20.737 | −1.895 | 1.00 | 47.40 | C |
| ATOM | 1467 | CG | ASP | A | 184 | 27.176 | 21.121 | −2.919 | 1.00 | 51.79 | C |
| ATOM | 1468 | OD1 | ASP | A | 184 | 28.335 | 20.597 | −2.826 | 1.00 | 52.58 | O |
| ATOM | 1469 | OD2 | ASP | A | 184 | 26.837 | 21.913 | −3.810 | 1.00 | 50.09 | O |
| ATOM | 1470 | C | ASP | A | 184 | 25.511 | 19.152 | −0.009 | 1.00 | 41.54 | C |
| ATOM | 1471 | O | ASP | A | 184 | 25.878 | 19.550 | 1.096 | 1.00 | 46.33 | O |
| ATOM | 1472 | N | ALA | A | 185 | 24.374 | 18.482 | −0.205 | 1.00 | 41.06 | N |
| ATOM | 1473 | CA | ALA | A | 185 | 23.446 | 18.196 | 0.904 | 1.00 | 36.54 | C |
| ATOM | 1474 | CB | ALA | A | 185 | 22.163 | 17.677 | 0.341 | 1.00 | 41.53 | C |
| ATOM | 1475 | C | ALA | A | 185 | 24.114 | 17.213 | 1.870 | 1.00 | 36.31 | C |
| ATOM | 1476 | O | ALA | A | 185 | 23.974 | 17.374 | 3.131 | 1.00 | 34.57 | O |
| ATOM | 1477 | N | LEU | A | 186 | 24.844 | 16.230 | 1.341 | 1.00 | 35.80 | N |
| ATOM | 1478 | CA | LEU | A | 186 | 25.548 | 15.248 | 2.230 | 1.00 | 41.31 | C |
| ATOM | 1479 | CB | LEU | A | 186 | 26.138 | 14.095 | 1.413 | 1.00 | 38.78 | C |
| ATOM | 1480 | CG | LEU | A | 186 | 25.085 | 13.216 | 0.730 | 1.00 | 40.46 | C |
| ATOM | 1481 | CD1 | LEU | A | 186 | 25.731 | 12.018 | 0.066 | 1.00 | 42.35 | C |
| ATOM | 1482 | CD2 | LEU | A | 186 | 23.991 | 12.765 | 1.694 | 1.00 | 36.66 | C |
| ATOM | 1483 | C | LEU | A | 186 | 26.582 | 15.972 | 3.103 | 1.00 | 38.26 | C |
| ATOM | 1484 | O | LEU | A | 186 | 26.614 | 15.695 | 4.320 | 1.00 | 36.91 | O |
| ATOM | 1485 | N | VAL | A | 187 | 27.275 | 16.974 | 2.552 | 1.00 | 40.19 | N |
| ATOM | 1486 | CA | VAL | A | 187 | 28.235 | 17.829 | 3.320 | 1.00 | 38.70 | C |
| ATOM | 1487 | CB | VAL | A | 187 | 29.079 | 18.727 | 2.394 | 1.00 | 41.33 | C |
| ATOM | 1488 | CG1 | VAL | A | 187 | 30.013 | 19.623 | 3.191 | 1.00 | 45.28 | C |
| ATOM | 1489 | CG2 | VAL | A | 187 | 29.872 | 17.923 | 1.365 | 1.00 | 42.39 | C |
| ATOM | 1490 | C | VAL | A | 187 | 27.466 | 18.635 | 4.376 | 1.00 | 36.68 | C |
| ATOM | 1491 | O | VAL | A | 187 | 27.872 | 18.639 | 5.532 | 1.00 | 42.07 | O |
| ATOM | 1492 | N | ASP | A | 188 | 26.355 | 19.270 | 4.018 | 1.00 | 40.01 | N |
| ATOM | 1493 | CA | ASP | A | 188 | 25.578 | 20.117 | 4.963 | 1.00 | 36.42 | C |
| ATOM | 1494 | CB | ASP | A | 188 | 24.543 | 20.967 | 4.224 | 1.00 | 38.79 | C |
| ATOM | 1495 | CG | ASP | A | 188 | 25.115 | 21.873 | 3.142 | 1.00 | 40.41 | C |
| ATOM | 1496 | OD1 | ASP | A | 188 | 26.323 | 22.163 | 3.174 | 1.00 | 46.31 | O |
| ATOM | 1497 | OD2 | ASP | A | 188 | 24.356 | 22.272 | 2.263 | 1.00 | 42.31 | O |
| ATOM | 1498 | C | ASP | A | 188 | 24.976 | 19.252 | 6.080 | 1.00 | 36.29 | C |
| ATOM | 1499 | O | ASP | A | 188 | 24.685 | 19.841 | 7.137 | 1.00 | 37.12 | O |
| ATOM | 1500 | N | PHE | A | 189 | 24.835 | 17.932 | 5.882 | 1.00 | 38.44 | N |
| ATOM | 1501 | CA | PHE | A | 189 | 24.278 | 16.957 | 6.860 | 1.00 | 43.60 | C |
| ATOM | 1502 | CB | PHE | A | 189 | 23.535 | 15.800 | 6.180 | 1.00 | 47.70 | C |
| ATOM | 1503 | CG | PHE | A | 189 | 22.243 | 16.105 | 5.458 | 1.00 | 47.39 | C |
| ATOM | 1504 | CD1 | PHE | A | 189 | 21.648 | 17.354 | 5.512 | 1.00 | 46.03 | C |
| ATOM | 1505 | CE1 | PHE | A | 189 | 20.470 | 17.611 | 4.831 | 1.00 | 43.31 | C |
| ATOM | 1506 | CZ | PHE | A | 189 | 19.854 | 16.615 | 4.116 | 1.00 | 43.36 | C |
| ATOM | 1507 | CD2 | PHE | A | 189 | 21.610 | 15.109 | 4.728 | 1.00 | 44.51 | C |
| ATOM | 1508 | CE2 | PHE | A | 189 | 20.427 | 15.368 | 4.052 | 1.00 | 45.17 | C |
| ATOM | 1509 | C | PHE | A | 189 | 25.360 | 16.247 | 7.690 | 1.00 | 47.16 | C |
| ATOM | 1510 | O | PHE | A | 189 | 25.009 | 15.701 | 8.759 | 1.00 | 53.86 | O |
| ATOM | 1511 | N | THR | A | 190 | 26.609 | 16.147 | 7.233 | 1.00 | 46.69 | N |
| ATOM | 1512 | CA | THR | A | 190 | 27.619 | 15.323 | 7.972 | 1.00 | 48.63 | C |
| ATOM | 1513 | CB | THR | A | 190 | 28.108 | 14.163 | 7.101 | 1.00 | 45.93 | C |
| ATOM | 1514 | OG1 | THR | A | 190 | 28.922 | 14.779 | 6.110 | 1.00 | 38.54 | O |
| ATOM | 1515 | CG2 | THR | A | 190 | 26.992 | 13.356 | 6.474 | 1.00 | 47.42 | C |
| ATOM | 1516 | C | THR | A | 190 | 28.849 | 16.114 | 8.450 | 1.00 | 48.72 | C |
| ATOM | 1517 | O | THR | A | 190 | 29.508 | 15.615 | 9.362 | 1.00 | 51.42 | O |
| ATOM | 1518 | N | GLY | A | 191 | 29.179 | 17.249 | 7.818 | 1.00 | 51.04 | N |
| ATOM | 1519 | CA | GLY | A | 191 | 30.446 | 17.984 | 7.992 | 1.00 | 46.89 | C |
| ATOM | 1520 | C | GLY | A | 191 | 31.555 | 17.396 | 7.141 | 1.00 | 51.75 | C |
| ATOM | 1521 | O | GLY | A | 191 | 32.716 | 17.803 | 7.292 | 1.00 | 48.55 | O |
| ATOM | 1522 | N | GLY | A | 192 | 31.232 | 16.456 | 6.255 | 1.00 | 51.57 | N |
| ATOM | 1523 | CA | GLY | A | 192 | 32.261 | 15.664 | 5.561 | 1.00 | 44.05 | C |
| ATOM | 1524 | C | GLY | A | 192 | 32.790 | 16.412 | 4.366 | 1.00 | 42.23 | C |
| ATOM | 1525 | O | GLY | A | 192 | 32.615 | 17.615 | 4.319 | 1.00 | 50.58 | O |
| ATOM | 1526 | N | VAL | A | 193 | 33.337 | 15.698 | 3.387 | 1.00 | 44.48 | N |
| ATOM | 1527 | CA | VAL | A | 193 | 33.782 | 16.288 | 2.096 | 1.00 | 47.71 | C |
| ATOM | 1528 | CB | VAL | A | 193 | 35.312 | 16.451 | 2.051 | 1.00 | 47.20 | C |
| ATOM | 1529 | CG1 | VAL | A | 193 | 35.745 | 17.002 | 0.708 | 1.00 | 47.20 | C |
| ATOM | 1530 | CG2 | VAL | A | 193 | 35.808 | 17.330 | 3.183 | 1.00 | 47.39 | C |
| ATOM | 1531 | C | VAL | A | 193 | 33.284 | 15.406 | 0.954 | 1.00 | 46.61 | C |
| ATOM | 1532 | O | VAL | A | 193 | 33.529 | 14.208 | 0.989 | 1.00 | 44.80 | O |
| ATOM | 1533 | N | SER | A | 194 | 32.606 | 15.998 | −0.022 | 1.00 | 50.75 | N |
| ATOM | 1534 | CA | SER | A | 194 | 32.004 | 15.246 | −1.143 | 1.00 | 58.01 | C |
| ATOM | 1535 | CB | SER | A | 194 | 30.669 | 15.817 | −1.554 | 1.00 | 60.26 | C |
| ATOM | 1536 | OG | SER | A | 194 | 29.617 | 15.170 | −0.833 | 1.00 | 57.13 | O |
| ATOM | 1537 | C | SER | A | 194 | 33.022 | 15.194 | −2.277 | 1.00 | 58.85 | C |
| ATOM | 1538 | O | SER | A | 194 | 33.795 | 16.146 | −2.414 | 1.00 | 71.63 | O |
| ATOM | 1539 | N | GLU | A | 195 | 33.079 | 14.069 | −2.976 | 1.00 | 60.13 | N |
| ATOM | 1540 | CA | GLU | A | 195 | 33.950 | 13.867 | −4.154 | 1.00 | 68.37 | C |
| ATOM | 1541 | CB | GLU | A | 195 | 35.174 | 13.018 | −3.796 | 1.00 | 72.98 | C |
| ATOM | 1542 | CG | GLU | A | 195 | 36.006 | 12.690 | −5.030 | 1.00 | 86.21 | C |
| ATOM | 1543 | CD | GLU | A | 195 | 37.403 | 12.119 | −4.849 | 1.00 | 92.69 | C |
| ATOM | 1544 | OE1 | GLU | A | 195 | 38.051 | 11.848 | −5.897 | 1.00 | 81.40 | O |
| ATOM | 1545 | OE2 | GLU | A | 195 | 37.844 | 11.953 | −3.682 | 1.00 | 100.86 | O |
| ATOM | 1546 | C | GLU | A | 195 | 33.094 | 13.228 | −5.243 | 1.00 | 65.70 | C |
| ATOM | 1547 | O | GLU | A | 195 | 32.804 | 12.042 | −5.185 | 1.00 | 72.77 | O |
| ATOM | 1548 | N | PRO | A | 196 | 32.614 | 13.997 | −6.240 | 1.00 | 58.83 | N |
| ATOM | 1549 | CA | PRO | A | 196 | 31.852 | 13.425 | −7.346 | 1.00 | 65.51 | C |
| ATOM | 1550 | CB | PRO | A | 196 | 31.119 | 14.632 | −7.946 | 1.00 | 66.71 | C |
| ATOM | 1551 | CG | PRO | A | 196 | 31.982 | 15.816 | −7.587 | 1.00 | 65.35 | C |
| ATOM | 1552 | CD | PRO | A | 196 | 32.730 | 15.453 | −6.322 | 1.00 | 60.45 | C |
| ATOM | 1553 | C | PRO | A | 196 | 32.723 | 12.734 | −8.407 | 1.00 | 64.19 | C |
| ATOM | 1554 | O | PRO | A | 196 | 33.688 | 13.295 | −8.841 | 1.00 | 68.49 | O |
| ATOM | 1555 | N | ILE | A | 197 | 32.331 | 11.534 | −8.815 | 1.00 | 65.42 | N |
| ATOM | 1556 | CA | ILE | A | 197 | 33.018 | 10.749 | −9.872 | 1.00 | 61.43 | C |
| ATOM | 1557 | CB | ILE | A | 197 | 33.556 | 9.430 | −9.284 | 1.00 | 62.90 | C |
| ATOM | 1558 | CG1 | ILE | A | 197 | 34.680 | 9.687 | −8.278 | 1.00 | 66.60 | C |
| ATOM | 1559 | CG2 | ILE | A | 197 | 34.006 | 8.492 | −10.381 | 1.00 | 64.17 | C |
| ATOM | 1560 | CD1 | ILE | A | 197 | 34.261 | 9.547 | −6.840 | 1.00 | 70.39 | C |
| ATOM | 1561 | C | ILE | A | 197 | 32.033 | 10.548 | −11.024 | 1.00 | 61.44 | C |
| ATOM | 1562 | O | ILE | A | 197 | 30.917 | 10.063 | −10.786 | 1.00 | 66.19 | O |
| ATOM | 1563 | N | ASP | A | 198 | 32.436 | 10.936 | −12.232 | 1.00 | 68.37 | N |
| ATOM | 1564 | CA | ASP | A | 198 | 31.629 | 10.776 | −13.472 | 1.00 | 73.50 | C |
| ATOM | 1565 | CB | ASP | A | 198 | 31.662 | 12.045 | −14.330 | 1.00 | 69.21 | C |
| ATOM | 1566 | CG | ASP | A | 198 | 30.697 | 12.016 | −15.505 | 1.00 | 66.07 | C |
| ATOM | 1567 | OD1 | ASP | A | 198 | 30.418 | 10.910 | −16.003 | 1.00 | 57.04 | O |
| ATOM | 1568 | OD2 | ASP | A | 198 | 30.239 | 13.103 | −15.921 | 1.00 | 69.71 | O |
| ATOM | 1569 | C | ASP | A | 198 | 32.133 | 9.537 | −14.228 | 1.00 | 77.76 | C |
| ATOM | 1570 | O | ASP | A | 198 | 33.290 | 9.548 | −14.704 | 1.00 | 73.07 | O |
| ATOM | 1571 | N | LEU | A | 199 | 31.261 | 8.533 | −14.363 | 1.00 | 75.27 | N |
| ATOM | 1572 | CA | LEU | A | 199 | 31.570 | 7.209 | −14.958 | 1.00 | 76.74 | C |
| ATOM | 1573 | CB | LEU | A | 199 | 30.573 | 6.185 | −14.408 | 1.00 | 75.74 | C |
| ATOM | 1574 | CG | LEU | A | 199 | 30.359 | 6.220 | −12.895 | 1.00 | 79.12 | C |
| ATOM | 1575 | CD1 | LEU | A | 199 | 29.186 | 5.340 | −12.492 | 1.00 | 80.07 | C |
| ATOM | 1576 | CD2 | LEU | A | 199 | 31.617 | 5.806 | −12.146 | 1.00 | 72.77 | C |
| ATOM | 1577 | C | LEU | A | 199 | 31.517 | 7.269 | −16.493 | 1.00 | 78.95 | C |
| ATOM | 1578 | O | LEU | A | 199 | 31.827 | 6.234 | −17.114 | 1.00 | 85.32 | O |
| ATOM | 1579 | N | THR | A | 200 | 31.147 | 8.407 | −17.096 | 1.00 | 81.91 | N |
| ATOM | 1580 | CA | THR | A | 200 | 31.058 | 8.570 | −18.578 | 1.00 | 86.13 | C |
| ATOM | 1581 | CB | THR | A | 200 | 29.621 | 8.897 | −19.012 | 1.00 | 85.82 | C |
| ATOM | 1582 | OG1 | THR | A | 200 | 29.354 | 10.266 | −18.707 | 1.00 | 94.42 | O |
| ATOM | 1583 | CG2 | THR | A | 200 | 28.585 | 8.014 | −18.349 | 1.00 | 80.61 | C |
| ATOM | 1584 | C | THR | A | 200 | 32.068 | 9.612 | −19.100 | 1.00 | 90.43 | C |
| ATOM | 1585 | O | THR | A | 200 | 32.192 | 9.707 | −20.335 | 1.00 | 100.50 | O |
| ATOM | 1586 | N | GLU | A | 201 | 32.737 | 10.384 | −18.229 | 1.00 | 93.29 | N |
| ATOM | 1587 | CA | GLU | A | 201 | 33.937 | 11.204 | −18.587 | 1.00 | 94.62 | C |
| ATOM | 1588 | CB | GLU | A | 201 | 33.966 | 12.561 | −17.875 | 1.00 | 80.44 | C |
| ATOM | 1589 | C | GLU | A | 201 | 35.178 | 10.363 | −18.265 | 1.00 | 93.40 | C |
| ATOM | 1590 | O | GLU | A | 201 | 36.131 | 10.390 | −19.057 | 1.00 | 104.16 | O |
| ATOM | 1591 | N | GLY | A | 202 | 35.150 | 9.639 | −17.143 | 1.00 | 92.42 | N |
| ATOM | 1592 | CA | GLY | A | 202 | 35.980 | 8.443 | −16.921 | 1.00 | 93.99 | C |
| ATOM | 1593 | C | GLY | A | 202 | 35.475 | 7.309 | −17.791 | 1.00 | 96.29 | C |
| ATOM | 1594 | O | GLY | A | 202 | 34.391 | 7.471 | −18.384 | 1.00 | 95.63 | O |
| ATOM | 1595 | N | ASP | A | 203 | 36.216 | 6.204 | −17.884 | 1.00 | 102.23 | N |
| ATOM | 1596 | CA | ASP | A | 203 | 35.842 | 5.056 | −18.753 | 1.00 | 110.54 | C |
| ATOM | 1597 | CB | ASP | A | 203 | 36.767 | 4.959 | −19.973 | 1.00 | 118.15 | C |
| ATOM | 1598 | CG | ASP | A | 203 | 36.187 | 4.161 | −21.133 | 1.00 | 121.66 | C |
| ATOM | 1599 | OD1 | ASP | A | 203 | 35.001 | 3.782 | −21.051 | 1.00 | 130.04 | O |
| ATOM | 1600 | OD2 | ASP | A | 203 | 36.924 | 3.937 | −22.118 | 1.00 | 116.70 | O |
| ATOM | 1601 | C | ASP | A | 203 | 35.838 | 3.782 | −17.905 | 1.00 | 109.43 | C |
| ATOM | 1602 | O | ASP | A | 203 | 36.455 | 2.788 | −18.326 | 1.00 | 109.53 | O |
| ATOM | 1603 | N | PHE | A | 204 | 35.147 | 3.809 | −16.762 | 1.00 | 105.80 | N |
| ATOM | 1604 | CA | PHE | A | 204 | 35.110 | 2.691 | −15.779 | 1.00 | 96.94 | C |
| ATOM | 1605 | CB | PHE | A | 204 | 34.507 | 3.166 | −14.456 | 1.00 | 86.63 | C |
| ATOM | 1606 | CG | PHE | A | 204 | 35.249 | 4.299 | −13.793 | 1.00 | 81.44 | C |
| ATOM | 1607 | CD1 | PHE | A | 204 | 36.310 | 4.056 | −12.933 | 1.00 | 82.60 | C |
| ATOM | 1608 | CE1 | PHE | A | 204 | 36.975 | 5.101 | −12.310 | 1.00 | 80.21 | C |
| ATOM | 1609 | CZ | PHE | A | 204 | 36.580 | 6.400 | −12.532 | 1.00 | 77.52 | C |
| ATOM | 1610 | CD2 | PHE | A | 204 | 34.865 | 5.612 | −14.006 | 1.00 | 78.53 | C |
| ATOM | 1611 | CE2 | PHE | A | 204 | 35.523 | 6.657 | −13.375 | 1.00 | 76.25 | C |
| ATOM | 1612 | C | PHE | A | 204 | 34.352 | 1.495 | −16.379 | 1.00 | 91.12 | C |
| ATOM | 1613 | O | PHE | A | 204 | 34.712 | 0.331 | −16.098 | 1.00 | 81.79 | O |
| ATOM | 1614 | N | ALA | A | 205 | 33.337 | 1.773 | −17.199 | 1.00 | 97.57 | N |
| ATOM | 1615 | CA | ALA | A | 205 | 32.585 | 0.761 | −17.977 | 1.00 | 106.62 | C |
| ATOM | 1616 | CB | ALA | A | 205 | 31.493 | 1.430 | −18.783 | 1.00 | 97.87 | C |
| ATOM | 1617 | C | ALA | A | 205 | 33.564 | −0.019 | −18.867 | 1.00 | 117.68 | C |
| ATOM | 1618 | O | ALA | A | 205 | 33.565 | −1.259 | −18.782 | 1.00 | 120.65 | O |
| ATOM | 1619 | N | ASN | A | 206 | 34.397 | 0.690 | −19.644 | 1.00 | 128.80 | N |
| ATOM | 1620 | CA | ASN | A | 206 | 35.252 | 0.118 | −20.723 | 1.00 | 121.58 | C |
| ATOM | 1621 | CB | ASN | A | 206 | 34.937 | 0.769 | −22.070 | 1.00 | 126.98 | C |
| ATOM | 1622 | CG | ASN | A | 206 | 33.449 | 0.863 | −22.338 | 1.00 | 128.78 | C |
| ATOM | 1623 | OD1 | ASN | A | 206 | 32.807 | −0.138 | −22.651 | 1.00 | 119.24 | O |
| ATOM | 1624 | ND2 | ASN | A | 206 | 32.893 | 2.059 | −22.210 | 1.00 | 129.64 | N |
| ATOM | 1625 | C | ASN | A | 206 | 36.737 | 0.260 | −20.360 | 1.00 | 115.92 | C |
| ATOM | 1626 | O | ASN | A | 206 | 37.564 | 0.466 | −21.276 | 1.00 | 114.17 | O |
| ATOM | 1627 | N | ASP | A | 207 | 37.057 | 0.149 | −19.070 | 1.00 | 108.60 | N |
| ATOM | 1628 | CA | ASP | A | 207 | 38.444 | 0.110 | −18.536 | 1.00 | 102.22 | C |
| ATOM | 1629 | CB | ASP | A | 207 | 39.092 | 1.498 | −18.462 | 1.00 | 99.41 | C |
| ATOM | 1630 | CG | ASP | A | 207 | 40.499 | 1.510 | −17.881 | 1.00 | 91.72 | C |
| ATOM | 1631 | OD1 | ASP | A | 207 | 40.670 | 1.063 | −16.727 | 1.00 | 86.42 | O |
| ATOM | 1632 | OD2 | ASP | A | 207 | 41.414 | 1.968 | −18.587 | 1.00 | 90.89 | O |
| ATOM | 1633 | C | ASP | A | 207 | 38.371 | −0.550 | −17.163 | 1.00 | 99.94 | C |
| ATOM | 1634 | O | ASP | A | 207 | 37.799 | 0.063 | −16.241 | 1.00 | 109.65 | O |
| ATOM | 1635 | N | GLU | A | 208 | 38.930 | −1.754 | −17.060 | 1.00 | 99.80 | N |
| ATOM | 1636 | CA | GLU | A | 208 | 38.925 | −2.595 | −15.838 | 1.00 | 97.61 | C |
| ATOM | 1637 | CB | GLU | A | 208 | 39.330 | −4.014 | −16.238 | 1.00 | 96.28 | C |
| ATOM | 1638 | CG | GLU | A | 208 | 39.314 | −5.009 | −15.100 | 1.00 | 97.39 | C |
| ATOM | 1639 | CD | GLU | A | 208 | 39.578 | −6.435 | −15.549 | 1.00 | 102.76 | C |
| ATOM | 1640 | OE1 | GLU | A | 208 | 40.235 | −6.613 | −16.593 | 1.00 | 108.35 | O |
| ATOM | 1641 | OE2 | GLU | A | 208 | 39.118 | −7.361 | −14.859 | 1.00 | 112.08 | O |
| ATOM | 1642 | C | GLU | A | 208 | 39.853 | −1.987 | −14.774 | 1.00 | 84.84 | C |
| ATOM | 1643 | O | GLU | A | 208 | 39.497 | −2.008 | −13.595 | 1.00 | 78.49 | O |
| ATOM | 1644 | N | THR | A | 209 | 41.006 | −1.463 | −15.177 | 1.00 | 84.47 | N |
| ATOM | 1645 | CA | THR | A | 209 | 42.120 | −1.095 | −14.263 | 1.00 | 88.41 | C |
| ATOM | 1646 | CB | THR | A | 209 | 43.431 | −1.028 | −15.066 | 1.00 | 87.98 | C |
| ATOM | 1647 | OG1 | THR | A | 209 | 44.489 | −1.128 | −14.119 | 1.00 | 81.21 | O |
| ATOM | 1648 | CG2 | THR | A | 209 | 43.594 | 0.217 | −15.914 | 1.00 | 89.61 | C |
| ATOM | 1649 | C | THR | A | 209 | 41.729 | 0.154 | −13.448 | 1.00 | 84.15 | C |
| ATOM | 1650 | O | THR | A | 209 | 41.891 | 0.134 | −12.204 | 1.00 | 81.11 | O |
| ATOM | 1651 | N | LYS | A | 210 | 41.207 | 1.193 | −14.105 | 1.00 | 80.90 | N |
| ATOM | 1652 | CA | LYS | A | 210 | 40.692 | 2.419 | −13.431 | 1.00 | 85.49 | C |
| ATOM | 1653 | CB | LYS | A | 210 | 40.295 | 3.479 | −14.468 | 1.00 | 81.93 | C |
| ATOM | 1654 | C | LYS | A | 210 | 39.527 | 2.033 | −12.498 | 1.00 | 75.67 | C |
| ATOM | 1655 | O | LYS | A | 210 | 39.500 | 2.515 | −11.357 | 1.00 | 74.93 | O |
| ATOM | 1656 | N | ARG | A | 211 | 38.625 | 1.161 | −12.956 | 1.00 | 76.26 | N |
| ATOM | 1657 | CA | ARG | A | 211 | 37.450 | 0.653 | −12.186 | 1.00 | 78.19 | C |
| ATOM | 1658 | CB | ARG | A | 211 | 36.602 | −0.260 | −13.080 | 1.00 | 81.53 | C |
| ATOM | 1659 | CG | ARG | A | 211 | 35.586 | −1.121 | −12.342 | 1.00 | 82.31 | C |
| ATOM | 1660 | CD | ARG | A | 211 | 34.496 | −1.609 | −13.283 | 1.00 | 89.07 | C |
| ATOM | 1661 | NE | ARG | A | 211 | 34.965 | −2.282 | −14.500 | 1.00 | 83.70 | N |
| ATOM | 1662 | CZ | ARG | A | 211 | 35.058 | −3.600 | −14.667 | 1.00 | 77.79 | C |
| ATOM | 1663 | NH1 | ARG | A | 211 | 34.741 | −4.428 | −13.686 | 1.00 | 75.61 | N |
| ATOM | 1664 | NH2 | ARG | A | 211 | 35.472 | −4.084 | −15.826 | 1.00 | 80.38 | N |
| ATOM | 1665 | C | ARG | A | 211 | 37.911 | −0.041 | −10.894 | 1.00 | 69.46 | C |
| ATOM | 1666 | O | ARG | A | 211 | 37.418 | 0.329 | −9.822 | 1.00 | 65.45 | O |
| ATOM | 1667 | N | ASN | A | 212 | 38.819 | −1.010 | −10.973 | 1.00 | 66.19 | N |
| ATOM | 1668 | CA | ASN | A | 212 | 39.334 | −1.701 | −9.764 | 1.00 | 68.83 | C |
| ATOM | 1669 | CB | ASN | A | 212 | 40.404 | −2.742 | −10.089 | 1.00 | 70.92 | C |
| ATOM | 1670 | CG | ASN | A | 212 | 39.818 | −4.040 | −10.599 | 1.00 | 77.20 | C |
| ATOM | 1671 | OD1 | ASN | A | 212 | 39.876 | −4.318 | −11.794 | 1.00 | 79.17 | O |
| ATOM | 1672 | ND2 | ASN | A | 212 | 39.250 | −4.835 | −9.704 | 1.00 | 80.83 | N |
| ATOM | 1673 | C | ASN | A | 212 | 39.879 | −0.639 | −8.811 | 1.00 | 75.21 | C |
| ATOM | 1674 | O | ASN | A | 212 | 39.737 | −0.813 | −7.585 | 1.00 | 75.90 | O |
| ATOM | 1675 | N | GLN | A | 213 | 40.481 | 0.419 | −9.358 | 1.00 | 73.09 | N |
| ATOM | 1676 | CA | GLN | A | 213 | 41.086 | 1.505 | −8.551 | 1.00 | 80.68 | C |
| ATOM | 1677 | CB | GLN | A | 213 | 41.937 | 2.403 | −9.452 | 1.00 | 95.43 | C |
| ATOM | 1678 | CG | GLN | A | 213 | 42.546 | 3.596 | −8.730 | 1.00 | 105.73 | C |
| ATOM | 1679 | CD | GLN | A | 213 | 43.886 | 3.985 | −9.306 | 1.00 | 110.78 | C |
| ATOM | 1680 | OE1 | GLN | A | 213 | 44.230 | 3.620 | −10.429 | 1.00 | 105.95 | O |
| ATOM | 1681 | NE2 | GLN | A | 213 | 44.661 | 4.731 | −8.534 | 1.00 | 113.30 | N |
| ATOM | 1682 | C | GLN | A | 213 | 39.969 | 2.242 | −7.795 | 1.00 | 68.83 | C |
| ATOM | 1683 | O | GLN | A | 213 | 40.124 | 2.458 | −6.582 | 1.00 | 59.22 | O |
| ATOM | 1684 | N | LEU | A | 214 | 38.867 | 2.577 | −8.470 | 1.00 | 60.15 | N |
| ATOM | 1685 | CA | LEU | A | 214 | 37.686 | 3.239 | −7.841 | 1.00 | 53.76 | C |
| ATOM | 1686 | CB | LEU | A | 214 | 36.648 | 3.564 | −8.915 | 1.00 | 52.18 | C |
| ATOM | 1687 | CG | LEU | A | 214 | 35.384 | 4.265 | −8.416 | 1.00 | 53.07 | C |
| ATOM | 1688 | CD1 | LEU | A | 214 | 35.716 | 5.457 | −7.533 | 1.00 | 53.38 | C |
| ATOM | 1689 | CD2 | LEU | A | 214 | 34.514 | 4.691 | −9.589 | 1.00 | 55.08 | C |
| ATOM | 1690 | C | LEU | A | 214 | 37.078 | 2.333 | −6.761 | 1.00 | 55.41 | C |
| ATOM | 1691 | O | LEU | A | 214 | 36.755 | 2.832 | −5.658 | 1.00 | 50.43 | O |
| ATOM | 1692 | N | PHE | A | 215 | 36.925 | 1.041 | −7.043 | 1.00 | 53.44 | N |
| ATOM | 1693 | CA | PHE | A | 215 | 36.277 | 0.108 | −6.093 | 1.00 | 55.02 | C |
| ATOM | 1694 | CB | PHE | A | 215 | 36.084 | −1.279 | −6.709 | 1.00 | 55.91 | C |
| ATOM | 1695 | CG | PHE | A | 215 | 35.487 | −2.258 | −5.737 | 1.00 | 54.31 | C |
| ATOM | 1696 | CD1 | PHE | A | 215 | 34.122 | −2.260 | −5.490 | 1.00 | 55.58 | C |
| ATOM | 1697 | CE1 | PHE | A | 215 | 33.573 | −3.138 | −4.569 | 1.00 | 51.97 | C |
| ATOM | 1698 | CZ | PHE | A | 215 | 34.387 | −4.003 | −3.875 | 1.00 | 55.44 | C |
| ATOM | 1699 | CD2 | PHE | A | 215 | 36.297 | −3.117 | −5.013 | 1.00 | 55.81 | C |
| ATOM | 1700 | CE2 | PHE | A | 215 | 35.745 | −3.992 | −4.090 | 1.00 | 56.86 | C |
| ATOM | 1701 | C | PHE | A | 215 | 37.069 | 0.107 | −4.777 | 1.00 | 55.36 | C |
| ATOM | 1702 | O | PHE | A | 215 | 36.429 | 0.092 | −3.718 | 1.00 | 60.56 | O |
| ATOM | 1703 | N | GLU | A | 216 | 38.407 | 0.139 | −4.826 | 1.00 | 60.04 | N |
| ATOM | 1704 | CA | GLU | A | 216 | 39.262 | 0.173 | −3.604 | 1.00 | 60.74 | C |
| ATOM | 1705 | CB | GLU | A | 216 | 40.748 | 0.075 | −3.962 | 1.00 | 63.54 | C |
| ATOM | 1706 | CG | GLU | A | 216 | 41.132 | −1.208 | −4.683 | 1.00 | 68.74 | C |
| ATOM | 1707 | CD | GLU | A | 216 | 40.771 | −2.515 | −3.993 | 1.00 | 77.69 | C |
| ATOM | 1708 | OE1 | GLU | A | 216 | 40.236 | −3.410 | −4.688 | 1.00 | 73.89 | O |
| ATOM | 1709 | OE2 | GLU | A | 216 | 41.008 | −2.637 | −2.762 | 1.00 | 83.28 | O |
| ATOM | 1710 | C | GLU | A | 216 | 38.956 | 1.457 | −2.816 | 1.00 | 57.84 | C |
| ATOM | 1711 | O | GLU | A | 216 | 38.785 | 1.395 | −1.584 | 1.00 | 53.06 | O |
| ATOM | 1712 | N | ARG | A | 217 | 38.863 | 2.586 | −3.512 | 1.00 | 60.70 | N |
| ATOM | 1713 | CA | ARG | A | 217 | 38.567 | 3.910 | −2.908 | 1.00 | 63.68 | C |
| ATOM | 1714 | CB | ARG | A | 217 | 38.686 | 4.998 | −3.991 | 1.00 | 75.02 | C |
| ATOM | 1715 | CG | ARG | A | 217 | 39.668 | 6.127 | −3.676 | 1.00 | 85.92 | C |
| ATOM | 1716 | CD | ARG | A | 217 | 39.080 | 7.525 | −3.879 | 1.00 | 91.32 | C |
| ATOM | 1717 | NE | ARG | A | 217 | 39.552 | 8.575 | −2.962 | 1.00 | 96.79 | N |
| ATOM | 1718 | CZ | ARG | A | 217 | 39.390 | 8.603 | −1.624 | 1.00 | 99.92 | C |
| ATOM | 1719 | NH1 | ARG | A | 217 | 38.804 | 7.607 | −0.976 | 1.00 | 97.44 | N |
| ATOM | 1720 | NH2 | ARG | A | 217 | 39.843 | 9.634 | −0.927 | 1.00 | 97.11 | N |
| ATOM | 1721 | C | ARG | A | 217 | 37.195 | 3.826 | −2.202 | 1.00 | 58.43 | C |
| ATOM | 1722 | O | ARG | A | 217 | 37.102 | 4.259 | −1.042 | 1.00 | 52.89 | O |
| ATOM | 1723 | N | MET | A | 218 | 36.181 | 3.216 | −2.833 | 1.00 | 54.97 | N |
| ATOM | 1724 | CA | MET | A | 218 | 34.800 | 3.139 | −2.278 | 1.00 | 53.21 | C |
| ATOM | 1725 | CB | MET | A | 218 | 33.785 | 2.697 | −3.328 | 1.00 | 52.30 | C |
| ATOM | 1726 | CG | MET | A | 218 | 33.499 | 3.775 | −4.334 | 1.00 | 55.59 | C |
| ATOM | 1727 | SD | MET | A | 218 | 32.657 | 3.077 | −5.763 | 1.00 | 66.37 | S |
| ATOM | 1728 | CE | MET | A | 218 | 30.969 | 3.040 | −5.152 | 1.00 | 67.30 | C |
| ATOM | 1729 | C | MET | A | 218 | 34.752 | 2.165 | −1.100 | 1.00 | 53.42 | C |
| ATOM | 1730 | O | MET | A | 218 | 34.110 | 2.516 | −0.073 | 1.00 | 52.56 | O |
| ATOM | 1731 | N | LEU | A | 219 | 35.390 | 0.995 | −1.237 | 1.00 | 52.56 | N |
| ATOM | 1732 | CA | LEU | A | 219 | 35.544 | 0.012 | −0.130 | 1.00 | 54.43 | C |
| ATOM | 1733 | CB | LEU | A | 219 | 36.406 | −1.170 | −0.590 | 1.00 | 59.59 | C |
| ATOM | 1734 | CG | LEU | A | 219 | 36.528 | −2.327 | 0.407 | 1.00 | 65.98 | C |
| ATOM | 1735 | CD1 | LEU | A | 219 | 35.256 | −3.173 | 0.463 | 1.00 | 66.94 | C |
| ATOM | 1736 | CD2 | LEU | A | 219 | 37.716 | −3.203 | 0.059 | 1.00 | 68.28 | C |
| ATOM | 1737 | C | LEU | A | 219 | 36.164 | 0.740 | 1.066 | 1.00 | 52.71 | C |
| ATOM | 1738 | O | LEU | A | 219 | 35.683 | 0.521 | 2.187 | 1.00 | 44.83 | O |
| ATOM | 1739 | N | LYS | A | 220 | 37.143 | 1.623 | 0.808 | 1.00 | 60.13 | N |
| ATOM | 1740 | CA | LYS | A | 220 | 37.888 | 2.405 | 1.831 | 1.00 | 61.38 | C |
| ATOM | 1741 | CB | LYS | A | 220 | 39.102 | 3.076 | 1.169 | 1.00 | 75.59 | C |
| ATOM | 1742 | CG | LYS | A | 220 | 39.979 | 3.940 | 2.075 | 1.00 | 85.60 | C |
| ATOM | 1743 | CD | LYS | A | 220 | 41.425 | 4.173 | 1.585 | 1.00 | 100.90 | C |
| ATOM | 1744 | CE | LYS | A | 220 | 41.570 | 4.974 | 0.298 | 1.00 | 102.67 | C |
| ATOM | 1745 | NZ | LYS | A | 220 | 41.345 | 6.429 | 0.492 | 1.00 | 106.67 | N |
| ATOM | 1746 | C | LYS | A | 220 | 36.917 | 3.379 | 2.521 | 1.00 | 52.56 | C |
| ATOM | 1747 | O | LYS | A | 220 | 36.784 | 3.305 | 3.766 | 1.00 | 48.07 | O |
| ATOM | 1748 | N | VAL | A | 221 | 36.232 | 4.244 | 1.769 | 1.00 | 46.89 | N |
| ATOM | 1749 | CA | VAL | A | 221 | 35.323 | 5.276 | 2.378 | 1.00 | 54.01 | C |
| ATOM | 175C | CB | VAL | A | 221 | 34.811 | 6.318 | 1.361 | 1.00 | 60.52 | C |
| ATOM | 1751 | CG1 | VAL | A | 221 | 34.354 | 5.687 | 0.063 | 1.00 | 71.79 | C |
| ATOM | 1752 | CG2 | VAL | A | 221 | 33.694 | 7.180 | 1.936 | 1.00 | 64.32 | C |
| ATOM | 1753 | C | VAL | A | 221 | 34.161 | 4.606 | 3.132 | 1.00 | 47.38 | C |
| ATOM | 1754 | O | VAL | A | 221 | 33.847 | 5.070 | 4.254 | 1.00 | 46.09 | O |
| ATOM | 1755 | N | HIS | A | 222 | 33.551 | 3.547 | 2.583 | 1.00 | 49.63 | N |
| ATOM | 1756 | CA | HIS | A | 222 | 32.402 | 2.853 | 3.227 | 1.00 | 48.90 | C |
| ATOM | 1757 | CB | HIS | A | 222 | 31.755 | 1.823 | 2.304 | 1.00 | 52.23 | C |
| ATOM | 1758 | CG | HIS | A | 222 | 30.560 | 1.193 | 2.937 | 1.00 | 52.18 | C |
| ATOM | 1759 | ND1 | HIS | A | 222 | 29.331 | 1.850 | 3.026 | 1.00 | 51.79 | N |
| ATOM | 1760 | CE1 | HIS | A | 222 | 28.470 | 1.063 | 3.649 | 1.00 | 50.70 | C |
| ATOM | 1761 | NE2 | HIS | A | 222 | 29.103 | −0.077 | 3.977 | 1.00 | 50.76 | N |
| ATOM | 1762 | CD2 | HIS | A | 222 | 30.415 | 0.012 | 3.575 | 1.00 | 48.31 | C |
| ATOM | 1763 | C | HIS | A | 222 | 32.809 | 2.200 | 4.552 | 1.00 | 48.30 | C |
| ATOM | 1764 | O | HIS | A | 222 | 32.002 | 2.252 | 5.503 | 1.00 | 48.43 | O |
| ATOM | 1765 | N | SER | A | 223 | 33.998 | 1.598 | 4.620 | 1.00 | 53.39 | N |
| ATOM | 1766 | CA | SER | A | 223 | 34.583 | 1.047 | 5.876 | 1.00 | 60.11 | C |
| ATOM | 1767 | CB | SER | A | 223 | 35.965 | 0.508 | 5.652 | 1.00 | 59.13 | C |
| ATOM | 1768 | OG | SER | A | 223 | 35.994 | −0.182 | 4.422 | 1.00 | 65.78 | O |
| ATOM | 1769 | C | SER | A | 223 | 34.602 | 2.122 | 6.962 | 1.00 | 56.22 | C |
| ATOM | 1770 | O | SER | A | 223 | 34.127 | 1.811 | 8.066 | 1.00 | 53.54 | O |
| ATOM | 1771 | N | ARG | A | 224 | 35.106 | 3.323 | 6.629 | 1.00 | 55.09 | N |
| ATOM | 1772 | CA | ARG | A | 224 | 35.317 | 4.470 | 7.561 | 1.00 | 57.56 | C |
| ATOM | 1773 | CB | ARG | A | 224 | 36.346 | 5.453 | 6.997 | 1.00 | 60.18 | C |
| ATOM | 1774 | CG | ARG | A | 224 | 37.777 | 4.940 | 6.950 | 1.00 | 66.07 | C |
| ATOM | 1775 | CD | ARG | A | 224 | 38.584 | 5.827 | 6.020 | 1.00 | 79.10 | C |
| ATOM | 1776 | NE | ARG | A | 224 | 40.022 | 5.841 | 6.273 | 1.00 | 88.60 | N |
| ATOM | 1777 | CZ | ARG | A | 224 | 40.882 | 4.900 | 5.880 | 1.00 | 100.09 | C |
| ATOM | 1778 | NH1 | ARG | A | 224 | 42.171 | 5.026 | 6.147 | 1.00 | 110.24 | N |
| ATOM | 1779 | NH2 | ARG | A | 224 | 40.461 | 3.827 | 5.234 | 1.00 | 101.34 | N |
| ATOM | 1780 | C | ARG | A | 224 | 34.014 | 5.242 | 7.823 | 1.00 | 56.84 | C |
| ATOM | 1781 | O | ARG | A | 224 | 34.098 | 6.327 | 8.448 | 1.00 | 62.65 | O |
| ATOM | 1782 | N | GLY | A | 225 | 32.859 | 4.731 | 7.384 | 1.00 | 51.99 | N |
| ATOM | 1783 | CA | GLY | A | 225 | 31.532 | 5.252 | 7.770 | 1.00 | 48.27 | C |
| ATOM | 1784 | C | GLY | A | 225 | 31.084 | 6.426 | 6.896 | 1.00 | 44.75 | C |
| ATOM | 1785 | O | GLY | A | 225 | 30.246 | 7.232 | 7.351 | 1.00 | 38.27 | O |
| ATOM | 1786 | N | GLY | A | 226 | 31.601 | 6.509 | 5.669 | 1.00 | 41.44 | N |
| ATOM | 1787 | CA | GLY | A | 226 | 31.280 | 7.584 | 4.721 | 1.00 | 41.04 | C |
| ATOM | 1788 | C | GLY | A | 226 | 29.984 | 7.287 | 4.001 | 1.00 | 42.86 | C |
| ATOM | 1789 | O | GLY | A | 226 | 29.389 | 6.230 | 4.259 | 1.00 | 43.22 | O |
| ATOM | 1790 | N | LEU | A | 227 | 29.540 | 8.195 | 3.141 | 1.00 | 38.63 | N |
| ATOM | 1791 | CA | LEU | A | 227 | 28.279 | 8.004 | 2.407 | 1.00 | 39.44 | C |
| ATOM | 1792 | CB | LEU | A | 227 | 27.351 | 9.182 | 2.684 | 1.00 | 38.89 | C |
| ATOM | 1793 | CG | LEU | A | 227 | 26.813 | 9.243 | 4.104 | 1.00 | 37.75 | C |
| ATOM | 1794 | CD1 | LEU | A | 227 | 25.942 | 10.472 | 4.280 | 1.00 | 37.70 | C |
| ATOM | 1795 | CD2 | LEU | A | 227 | 26.041 | 7.976 | 4.435 | 1.00 | 39.67 | C |
| ATOM | 1796 | C | LEU | A | 227 | 28.606 | 7.901 | 0.936 | 1.00 | 40.23 | C |
| ATOM | 1797 | O | LEU | A | 227 | 29.255 | 8.810 | 0.444 | 1.00 | 42.58 | O |
| ATOM | 1798 | N | ILE | A | 228 | 28.117 | 6.858 | 0.268 | 1.00 | 39.83 | N |
| ATOM | 1799 | CA | ILE | A | 228 | 28.205 | 6.760 | −1.214 | 1.00 | 38.46 | C |
| ATOM | 1800 | CB | ILE | A | 228 | 29.091 | 5.575 | −1.615 | 1.00 | 40.34 | C |
| ATOM | 1801 | CG1 | ILE | A | 228 | 30.450 | 5.669 | −0.918 | 1.00 | 38.49 | C |
| ATOM | 1802 | CG2 | ILE | A | 228 | 29.229 | 5.474 | −3.126 | 1.00 | 40.12 | C |
| ATOM | 1803 | CD1 | ILE | A | 228 | 31.050 | 4.334 | −0.612 | 1.00 | 43.58 | C |
| ATOM | 1804 | C | ILE | A | 228 | 26.797 | 6.668 | −1.790 | 1.00 | 39.47 | C |
| ATOM | 1805 | O | ILE | A | 228 | 25.923 | 6.056 | −1.167 | 1.00 | 42.45 | O |
| ATOM | 1806 | N | SER | A | 229 | 26.604 | 7.283 | −2.949 | 1.00 | 42.55 | N |
| ATOM | 1807 | CA | SER | A | 229 | 25.300 | 7.443 | −3.621 | 1.00 | 43.12 | C |
| ATOM | 1808 | CB | SER | A | 229 | 24.718 | 8.787 | −3.301 | 1.00 | 51.32 | C |
| ATOM | 1809 | OG | SER | A | 229 | 23.297 | 8.742 | −3.252 | 1.00 | 61.30 | O |
| ATOM | 1810 | C | SER | A | 229 | 25.574 | 7.267 | −5.104 | 1.00 | 47.97 | C |
| ATOM | 1811 | O | SER | A | 229 | 26.576 | 7.850 | −5.543 | 1.00 | 50.58 | O |
| ATOM | 1812 | N | ALA | A | 230 | 24.787 | 6.444 | −5.810 | 1.00 | 41.89 | N |
| ATOM | 1813 | CA | ALA | A | 230 | 24.934 | 6.214 | −7.260 | 1.00 | 37.56 | C |
| ATOM | 1814 | CB | ALA | A | 230 | 25.164 | 4.756 | −7.535 | 1.00 | 36.73 | C |
| ATOM | 1815 | C | ALA | A | 230 | 23.679 | 6.733 | −7.948 | 1.00 | 38.26 | C |
| ATOM | 1816 | O | ALA | A | 230 | 22.578 | 6.443 | −7.480 | 1.00 | 38.12 | O |
| ATOM | 1817 | N | SER | A | 231 | 23.849 | 7.493 | −9.019 | 1.00 | 42.84 | N |
| ATOM | 1818 | CA | SER | A | 231 | 22.743 | 8.158 | −9.745 | 1.00 | 45.92 | C |
| ATOM | 1819 | CB | SER | A | 231 | 22.691 | 9.624 | −9.458 | 1.00 | 48.93 | C |
| ATOM | 1820 | OG | SER | A | 231 | 23.956 | 10.223 | −9.694 | 1.00 | 49.21 | O |
| ATOM | 1821 | C | SER | A | 231 | 22.878 | 7.914 | −11.240 | 1.00 | 46.26 | C |
| ATOM | 1822 | O | SER | A | 231 | 23.991 | 7.569 | −11.709 | 1.00 | 44.51 | O |
| ATOM | 1823 | N | ILE | A | 232 | 21.755 | 8.086 | −11.924 | 1.00 | 47.04 | N |
| ATOM | 1824 | CA | ILE | A | 232 | 21.632 | 8.021 | −13.402 | 1.00 | 51.94 | C |
| ATOM | 1825 | CB | ILE | A | 232 | 20.579 | 6.961 | −13.792 | 1.00 | 55.46 | C |
| ATOM | 1826 | CG1 | ILE | A | 232 | 20.801 | 5.653 | −13.029 | 1.00 | 54.16 | C |
| ATOM | 1827 | CG2 | ILE | A | 232 | 20.553 | 6.767 | −15.305 | 1.00 | 56.75 | C |
| ATOM | 1828 | CD1 | ILE | A | 232 | 20.081 | 4.474 | −13.614 | 1.00 | 57.27 | C |
| ATOM | 1829 | C | ILE | A | 232 | 21.248 | 9.430 | −13.842 | 1.00 | 49.39 | C |
| ATOM | 1830 | O | ILE | A | 232 | 20.182 | 9.876 | −13.440 | 1.00 | 51.00 | O |
| ATOM | 1831 | N | LYS | A | 233 | 22.117 | 10.107 | −14.585 | 1.00 | 56.57 | N |
| ATOM | 1832 | CA | LYS | A | 233 | 21.923 | 11.500 | −15.058 | 1.00 | 61.10 | C |
| ATOM | 1833 | CB | LYS | A | 233 | 23.157 | 11.902 | −15.876 | 1.00 | 70.97 | C |
| ATOM | 1834 | CG | LYS | A | 233 | 23.424 | 13.396 | −16.029 | 1.00 | 80.99 | C |
| ATOM | 1835 | CD | LYS | A | 233 | 24.857 | 13.675 | −16.495 | 1.00 | 86.47 | C |
| ATOM | 1836 | CE | LYS | A | 233 | 25.525 | 14.863 | −15.827 | 1.00 | 91.64 | C |
| ATOM | 1837 | NZ | LYS | A | 233 | 25.284 | 16.124 | −16.566 | 1.00 | 98.92 | N |
| ATOM | 1838 | C | LYS | A | 233 | 20.619 | 11.559 | −15.866 | 1.00 | 57.37 | C |
| ATOM | 1839 | O | LYS | A | 233 | 20.438 | 10.743 | −16.765 | 1.00 | 55.61 | O |
| ATOM | 1840 | N | ALA | A | 234 | 19.717 | 12.471 | −15.536 | 1.00 | 61.81 | N |
| ATOM | 1841 | CA | ALA | A | 234 | 18.563 | 12.821 | −16.394 | 1.00 | 72.99 | C |
| ATOM | 1842 | CB | ALA | A | 234 | 17.345 | 13.126 | −15.549 | 1.00 | 74.31 | C |
| ATOM | 1843 | C | ALA | A | 234 | 18.994 | 14.007 | −17.263 | 1.00 | 74.40 | C |
| ATOM | 1844 | O | ALA | A | 234 | 19.148 | 15.111 | −16.713 | 1.00 | 76.04 | O |
| ATOM | 1845 | N | VAL | A | 235 | 19.251 | 13.777 | −18.552 | 1.00 | 77.41 | N |
| ATOM | 1846 | CA | VAL | A | 235 | 19.855 | 14.810 | −19.446 | 1.00 | 88.24 | C |
| ATOM | 1847 | CB | VAL | A | 235 | 20.750 | 14.195 | −20.540 | 1.00 | 97.65 | C |
| ATOM | 1848 | CG1 | VAL | A | 235 | 21.784 | 15.198 | −21.036 | 1.00 | 104.36 | C |
| ATOM | 1849 | CG2 | VAL | A | 235 | 21.451 | 12.927 | −20.065 | 1.00 | 99.05 | C |
| ATOM | 1850 | C | VAL | A | 235 | 18.719 | 15.676 | −20.014 | 1.00 | 91.23 | C |
| ATOM | 1851 | O | VAL | A | 235 | 18.669 | 16.874 | −19.645 | 1.00 | 80.23 | O |
| ATOM | 1852 | N | THR | A | 236 | 17.812 | 15.088 | −20.811 | 1.00 | 83.88 | N |
| ATOM | 1853 | CA | THR | A | 236 | 16.628 | 15.776 | −21.401 | 1.00 | 79.68 | C |
| ATOM | 1854 | CB | THR | A | 236 | 16.106 | 15.072 | −22.661 | 1.00 | 85.45 | C |
| ATOM | 1855 | OG1 | THR | A | 236 | 15.294 | 13.968 | −22.256 | 1.00 | 93.50 | O |
| ATOM | 1856 | CG2 | THR | A | 236 | 17.202 | 14.604 | −23.597 | 1.00 | 83.56 | C |
| ATOM | 1857 | C | THR | A | 236 | 15.509 | 15.855 | −20.357 | 1.00 | 80.25 | C |
| ATOM | 1858 | O | THR | A | 236 | 15.576 | 15.120 | −19.369 | 1.00 | 79.84 | O |
| ATOM | 1859 | N | ALA | A | 237 | 14.514 | 16.719 | −20.572 | 1.00 | 88.14 | N |
| ATOM | 1860 | CA | ALA | A | 237 | 13.324 | 16.863 | −19.697 | 1.00 | 86.81 | C |
| ATOM | 1861 | CB | ALA | A | 237 | 12.440 | 17.981 | −20.200 | 1.00 | 90.33 | C |
| ATOM | 1862 | C | ALA | A | 237 | 12.566 | 15.528 | −19.643 | 1.00 | 80.37 | C |
| ATOM | 1863 | O | ALA | A | 237 | 12.139 | 15.134 | −18.552 | 1.00 | 83.83 | O |
| ATOM | 1864 | N | ALA | A | 238 | 12.447 | 14.842 | −20.781 | 1.00 | 72.93 | N |
| ATOM | 1865 | CA | ALA | A | 238 | 11.675 | 13.588 | −20.943 | 1.00 | 78.31 | C |
| ATOM | 1866 | CB | ALA | A | 238 | 11.291 | 13.392 | −22.390 | 1.00 | 79.61 | C |
| ATOM | 1867 | C | ALA | A | 238 | 12.483 | 12.390 | −20.444 | 1.00 | 80.79 | C |
| ATOM | 1868 | O | ALA | A | 238 | 11.983 | 11.259 | −20.591 | 1.00 | 85.03 | O |
| ATOM | 1869 | N | ASP | A | 239 | 13.694 | 12.614 | −19.925 | 1.00 | 81.14 | N |
| ATOM | 1870 | CA | ASP | A | 239 | 14.524 | 11.545 | −19.303 | 1.00 | 80.03 | C |
| ATOM | 1871 | CB | ASP | A | 239 | 16.023 | 11.799 | −19.503 | 1.00 | 76.65 | C |
| ATOM | 1872 | CG | ASP | A | 239 | 16.545 | 11.320 | −20.850 | 1.00 | 77.87 | C |
| ATOM | 1873 | OD1 | ASP | A | 239 | 16.164 | 10.198 | −21.272 | 1.00 | 66.18 | O |
| ATOM | 1874 | OD2 | ASP | A | 239 | 17.335 | 12.065 | −21.466 | 1.00 | 80.65 | O |
| ATOM | 1875 | C | ASP | A | 239 | 14.124 | 11.407 | −17.828 | 1.00 | 71.53 | C |
| ATOM | 1876 | O | ASP | A | 239 | 14.057 | 10.279 | −17.332 | 1.00 | 68.07 | O |
| ATOM | 1877 | N | MET | A | 240 | 13.837 | 12.522 | −17.169 | 1.00 | 3.54 | N |
| ATOM | 1878 | CA | MET | A | 240 | 13.465 | 12.565 | −15.738 | 1.00 | 64.54 | C |
| ATOM | 1879 | CB | MET | A | 240 | 12.842 | 13.925 | −15.397 | 1.00 | 67.58 | C |
| ATOM | 1880 | CG | MET | A | 240 | 12.860 | 14.240 | −13.925 | 1.00 | 68.58 | C |
| ATOM | 1881 | SD | MET | A | 240 | 14.535 | 14.228 | −13.259 | 1.00 | 66.17 | S |
| ATOM | 1882 | CE | MET | A | 240 | 14.191 | 13.724 | −11.574 | 1.00 | 65.17 | C |
| ATOM | 1883 | C | MET | A | 240 | 12.473 | 11.435 | −15.448 | 1.00 | 55.39 | C |
| ATOM | 1884 | O | MET | A | 240 | 11.400 | 11.460 | −16.017 | 1.00 | 56.33 | O |
| ATOM | 1885 | N | GLU | A | 241 | 12.864 | 10.461 | −14.624 | 1.00 | 55.44 | N |
| ATOM | 1886 | CA | GLU | A | 241 | 12.026 | 9.331 | −14.119 | 1.00 | 57.36 | C |
| ATOM | 1887 | CB | GLU | A | 241 | 10.822 | 9.843 | −13.331 | 1.00 | 56.80 | C |
| ATOM | 1888 | CG | GLU | A | 241 | 11.255 | 10.550 | −12.062 | 1.00 | 61.75 | C |
| ATOM | 1889 | CD | GLU | A | 241 | 10.148 | 10.749 | −11.050 | 1.00 | 64.84 | C |
| ATOM | 1890 | OE1 | GLU | A | 241 | 9.173 | 11.442 | −11.399 | 1.00 | 66.85 | O |
| ATOM | 1891 | OE2 | GLU | A | 241 | 10.262 | 10.207 | −9.922 | 1.00 | 64.53 | O |
| ATOM | 1892 | C | GLU | A | 241 | 11.605 | 8.372 | −15.241 | 1.00 | 55.80 | C |
| ATOM | 1893 | O | GLU | A | 241 | 10.627 | 7.627 | −15.044 | 1.00 | 62.37 | O |
| ATOM | 1894 | N | ALA | A | 242 | 12.346 | 8.322 | −16.345 | 1.00 | 52.80 | N |
| ATOM | 1895 | CA | ALA | A | 242 | 12.214 | 7.248 | −17.350 | 1.00 | 54.98 | C |
| ATOM | 1896 | CB | ALA | A | 242 | 12.751 | 7.688 | −18.689 | 1.00 | 56.40 | C |
| ATOM | 1897 | C | ALA | A | 242 | 12.942 | 6.010 | −16.808 | 1.00 | 51.34 | C |
| ATOM | 1898 | O | ALA | A | 242 | 14.077 | 6.137 | −16.326 | 1.00 | 47.68 | O |
| ATOM | 1899 | N | ARG | A | 243 | 12.248 | 4.880 | −16.825 | 1.00 | 51.80 | N |
| ATOM | 1900 | CA | ARG | A | 243 | 12.754 | 3.539 | −16.456 | 1.00 | 51.03 | C |
| ATOM | 1901 | CB | ARG | A | 243 | 11.570 | 2.565 | −16.356 | 1.00 | 56.09 | C |
| ATOM | 1902 | CG | ARG | A | 243 | 11.200 | 2.114 | −14.944 | 1.00 | 63.05 | C |
| ATOM | 1903 | CD | ARG | A | 243 | 10.889 | 0.618 | −14.842 | 1.00 | 74.18 | C |
| ATOM | 1904 | NE | ARG | A | 243 | 11.542 | −0.178 | −15.892 | 1.00 | 82.82 | N |
| ATOM | 1905 | CZ | ARG | A | 243 | 11.342 | −1.469 | −16.154 | 1.00 | 69.54 | C |
| ATOM | 1906 | NH1 | ARG | A | 243 | 10.506 | −2.195 | −15.434 | 1.00 | 66.08 | N |
| ATOM | 1907 | NH2 | ARG | A | 243 | 12.002 | −2.026 | −17.150 | 1.00 | 73.75 | N |
| ATOM | 1908 | C | ARG | A | 243 | 13.767 | 3.109 | −17.518 | 1.00 | 48.95 | C |
| ATOM | 1909 | O | ARG | A | 243 | 13.536 | 3.385 | −18.688 | 1.00 | 62.09 | O |
| ATOM | 1910 | N | LEU | A | 244 | 14.883 | 2.519 | −17.108 | 1.00 | 49.83 | N |
| ATOM | 1911 | CA | LEU | A | 244 | 15.762 | 1.718 | −17.987 | 1.00 | 51.37 | C |
| ATOM | 1912 | CB | LEU | A | 244 | 17.183 | 1.675 | −17.409 | 1.00 | 54.58 | C |
| ATOM | 1913 | CG | LEU | A | 244 | 17.926 | 3.007 | −17.292 | 1.00 | 57.85 | C |
| ATOM | 1914 | CD1 | LEU | A | 244 | 19.394 | 2.781 | −16.946 | 1.00 | 55.85 | C |
| ATOM | 1915 | CD2 | LEU | A | 244 | 17.828 | 3.812 | −18.575 | 1.00 | 60.41 | C |
| ATOM | 1916 | C | LEU | A | 244 | 15.171 | 0.304 | −18.083 | 1.00 | 56.41 | C |
| ATOM | 1917 | O | LEU | A | 244 | 14.312 | −0.068 | −17.225 | 1.00 | 48.94 | O |
| ATOM | 1918 | N | ALA | A | 245 | 15.636 | −0.464 | −19.074 | 1.00 | 56.87 | N |
| ATOM | 1919 | CA | ALA | A | 245 | 15.264 | −1.880 | −19.279 | 1.00 | 58.13 | C |
| ATOM | 1920 | CB | ALA | A | 245 | 15.979 | −2.442 | −20.484 | 1.00 | 60.42 | C |
| ATOM | 1921 | C | ALA | A | 245 | 15.597 | −2.666 | −18.005 | 1.00 | 55.84 | C |
| ATOM | 1922 | O | ALA | A | 245 | 14.814 | −3.567 | −17.683 | 1.00 | 52.19 | O |
| ATOM | 1923 | N | CYS | A | 246 | 16.687 | −2.317 | −17.300 | 1.00 | 50.04 | N |
| ATOM | 1924 | CA | CYS | A | 246 | 17.181 | −3.051 | −16.100 | 1.00 | 48.17 | C |
| ATOM | 1925 | CB | CYS | A | 246 | 18.665 | −2.820 | −15.856 | 1.00 | 45.63 | C |
| ATOM | 1926 | SG | CYS | A | 246 | 19.039 | −1.129 | −15.339 | 1.00 | 45.62 | S |
| ATOM | 1927 | C | CYS | A | 246 | 16.381 | −2.650 | −14.853 | 1.00 | 48.70 | C |
| ATOM | 1928 | O | CYS | A | 246 | 16.595 | −3.274 | −13.769 | 1.00 | 46.57 | O |
| ATOM | 1929 | N | GLY | A | 247 | 15.495 | −1.657 | −14.976 | 1.00 | 46.40 | N |
| ATOM | 1930 | CA | GLY | A | 247 | 14.556 | −1.306 | −13.894 | 1.00 | 44.67 | C |
| ATOM | 1931 | C | GLY | A | 247 | 14.966 | −0.055 | −13.148 | 1.00 | 44.09 | C |
| ATOM | 1932 | O | GLY | A | 247 | 14.085 | 0.537 | −12.507 | 1.00 | 55.52 | O |
| ATOM | 1933 | N | LEU | A | 248 | 16.231 | 0.362 | −13.245 | 1.00 | 42.30 | N |
| ATOM | 1934 | CA | LEU | A | 248 | 16.749 | 1.570 | −12.553 | 1.00 | 42.56 | C |
| ATOM | 1935 | CB | LEU | A | 248 | 18.277 | 1.534 | −12.550 | 1.00 | 41.81 | C |
| ATOM | 1936 | CG | LEU | A | 248 | 18.890 | 0.361 | −11.779 | 1.00 | 39.63 | C |
| ATOM | 1937 | CD1 | LEU | A | 248 | 20.400 | 0.310 | −11.964 | 1.00 | 38.82 | C |
| ATOM | 1938 | CD2 | LEU | A | 248 | 18.550 | 0.435 | −10.304 | 1.00 | 38.20 | C |
| ATOM | 1939 | C | LEU | A | 248 | 16.194 | 2.821 | −13.244 | 1.00 | 46.10 | C |
| ATOM | 1940 | O | LEU | A | 248 | 15.851 | 2.724 | −14.430 | 1.00 | 48.43 | O |
| ATOM | 1941 | N | VAL | A | 249 | 16.095 | 3.943 | −12.524 | 1.00 | 43.12 | N |
| ATOM | 1942 | CA | VAL | A | 249 | 15.352 | 5.147 | −12.986 | 1.00 | 45.06 | C |
| ATOM | 1943 | CB | VAL | A | 249 | 14.223 | 5.493 | −11.997 | 1.00 | 48.97 | C |
| ATOM | 1944 | CG1 | VAL | A | 249 | 13.651 | 6.885 | −12.246 | 1.00 | 44.23 | C |
| ATOM | 1945 | CG2 | VAL | A | 249 | 13.123 | 4.438 | −12.025 | 1.00 | 49.51 | C |
| ATOM | 1946 | C | VAL | A | 249 | 16.318 | 6.322 | −13.184 | 1.00 | 46.87 | C |
| ATOM | 1947 | O | VAL | A | 249 | 17.020 | 6.673 | −12.233 | 1.00 | 44.32 | O |
| ATOM | 1948 | N | LYS | A | 250 | 16.299 | 6.939 | −14.368 | 1.00 | 50.81 | N |
| ATOM | 1949 | CA | LYS | A | 250 | 17.080 | 8.168 | −14.691 | 1.00 | 52.71 | C |
| ATOM | 1950 | CB | LYS | A | 250 | 16.910 | 8.538 | −16.169 | 1.00 | 57.39 | C |
| ATOM | 1951 | CG | LYS | A | 250 | 17.336 | 7.468 | −17.170 | 1.00 | 61.10 | C |
| ATOM | 1952 | CD | LYS | A | 250 | 17.274 | 7.957 | −18.604 | 1.00 | 68.21 | C |
| ATOM | 1953 | CE | LYS | A | 250 | 17.833 | 6.978 | −19.611 | 1.00 | 73.29 | C |
| ATOM | 1954 | NZ | LYS | A | 250 | 18.241 | 7.648 | −20.868 | 1.00 | 77.65 | N |
| ATOM | 1955 | C | LYS | A | 250 | 16.627 | 9.311 | −13.770 | 1.00 | 50.99 | C |
| ATOM | 1956 | O | LYS | A | 250 | 15.393 | 9.519 | −13.630 | 1.00 | 50.01 | O |
| ATOM | 1957 | N | GLY | A | 251 | 17.588 | 9.995 | −13.133 | 1.00 | 46.81 | N |
| ATOM | 1958 | CA | GLY | A | 251 | 17.352 | 11.153 | −12.250 | 1.00 | 42.29 | C |
| ATOM | 1959 | C | GLY | A | 251 | 17.016 | 10.734 | −10.829 | 1.00 | 41.61 | C |
| ATOM | 1960 | O | GLY | A | 251 | 16.595 | 11.581 | −10.006 | 1.00 | 40.97 | O |
| ATOM | 1961 | N | HIS | A | 252 | 17.147 | 9.450 | −10.539 | 1.00 | 41.37 | N |
| ATOM | 1962 | CA | HIS | A | 252 | 17.043 | 8.911 | −9.166 | 1.00 | 40.80 | C |
| ATOM | 1963 | CB | HIS | A | 252 | 16.115 | 7.692 | −9.139 | 1.00 | 40.46 | C |
| ATOM | 1964 | CG | HIS | A | 252 | 14.668 | 8.047 | −9.125 | 1.00 | 41.80 | C |
| ATOM | 1965 | ND1 | HIS | A | 252 | 13.684 | 7.123 | −8.861 | 1.00 | 40.51 | N |
| ATOM | 1966 | CE1 | HIS | A | 252 | 12.513 | 7.706 | −8.897 | 1.00 | 37.12 | C |
| ATOM | 1967 | NE2 | HIS | A | 252 | 12.698 | 8.980 | −9.188 | 1.00 | 40.27 | N |
| ATOM | 1968 | CD2 | HIS | A | 252 | 14.034 | 9.218 | −9.321 | 1.00 | 41.66 | C |
| ATOM | 1969 | C | HIS | A | 252 | 18.467 | 8.595 | −8.741 | 1.00 | 44.27 | C |
| ATOM | 1970 | O | HIS | A | 252 | 19.307 | 8.292 | −9.648 | 1.00 | 43.41 | O |
| ATOM | 1971 | N | ALA | A | 253 | 18.732 | 8.673 | −7.439 | 1.00 | 39.63 | N |
| ATOM | 1972 | CA | ALA | A | 253 | 20.021 | 8.272 | −6.847 | 1.00 | 39.33 | C |
| ATOM | 1973 | CB | ALA | A | 253 | 20.763 | 9.472 | −6.325 | 1.00 | 40.79 | C |
| ATOM | 1974 | C | ALA | A | 253 | 19.731 | 7.230 | −5.773 | 1.00 | 41.27 | C |
| ATOM | 1975 | O | ALA | A | 253 | 18.619 | 7.239 | −5.222 | 1.00 | 49.48 | O |
| ATOM | 1976 | N | TYR | A | 254 | 20.668 | 6.311 | −5.575 | 1.00 | 37.05 | N |
| ATOM | 1977 | CA | TYR | A | 254 | 20.477 | 5.036 | −4.846 | 1.00 | 34.63 | C |
| ATOM | 1978 | CB | TYR | A | 254 | 20.549 | 3.848 | −5.814 | 1.00 | 35.68 | C |
| ATOM | 1979 | CG | TYR | A | 254 | 19.449 | 3.811 | −6.841 | 1.00 | 33.93 | C |
| ATOM | 1980 | CD1 | TYR | A | 254 | 18.203 | 3.288 | −6.534 | 1.00 | 37.78 | C |
| ATOM | 1981 | CE1 | TYR | A | 254 | 17.163 | 3.272 | −7.449 | 1.00 | 35.15 | C |
| ATOM | 1982 | CZ | TYR | A | 254 | 17.369 | 3.768 | −8.722 | 1.00 | 36.64 | C |
| ATOM | 1983 | OH | TYR | A | 254 | 16.347 | 3.727 | −9.625 | 1.00 | 35.44 | O |
| ATOM | 1984 | CE2 | TYR | A | 254 | 18.611 | 4.292 | −9.051 | 1.00 | 37.02 | C |
| ATOM | 1985 | CD2 | TYR | A | 254 | 19.629 | 4.321 | −8.105 | 1.00 | 34.59 | C |
| ATOM | 1986 | C | TYR | A | 254 | 21.587 | 4.993 | −3.811 | 1.00 | 36.46 | C |
| ATOM | 1987 | O | TYR | A | 254 | 22.753 | 5.226 | −4.189 | 1.00 | 36.78 | O |
| ATOM | 1988 | N | ALA | A | 255 | 21.234 | 4.790 | −2.547 | 1.00 | 35.07 | N |
| ATOM | 1989 | CA | ALA | A | 255 | 22.197 | 4.861 | −1.434 | 1.00 | 36.04 | C |
| ATOM | 1990 | CB | ALA | A | 255 | 21.468 | 5.140 | −0.141 | 1.00 | 35.44 | C |
| ATOM | 1991 | C | ALA | A | 255 | 22.967 | 3.540 | −1.402 | 1.00 | 37.71 | C |
| ATOM | 1992 | O | ALA | A | 255 | 22.312 | 2.479 | −1.407 | 1.00 | 37.15 | O |
| ATOM | 1993 | N | VAL | A | 256 | 24.297 | 3.590 | −1.383 | 1.00 | 37.13 | N |
| ATOM | 1994 | CA | VAL | A | 256 | 25.123 | 2.366 | −1.168 | 1.00 | 37.75 | C |
| ATOM | 1995 | CB | VAL | A | 256 | 26.582 | 2.608 | −1.577 | 1.00 | 38.11 | C |
| ATOM | 1996 | CG1 | VAL | A | 256 | 27.476 | 1.495 | −1.069 | 1.00 | 40.41 | C |
| ATOM | 1997 | CG2 | VAL | A | 256 | 26.703 | 2.756 | −3.087 | 1.00 | 37.35 | C |
| ATOM | 1998 | C | VAL | A | 256 | 25.015 | 1.953 | 0.296 | 1.00 | 36.38 | C |
| ATOM | 1999 | O | VAL | A | 256 | 25.308 | 2.794 | 1.141 | 1.00 | 37.87 | O |
| ATOM | 2000 | N | THR | A | 257 | 24.606 | 0.718 | 0.607 | 1.00 | 37.36 | N |
| ATOM | 2001 | CA | THR | A | 257 | 24.599 | 0.247 | 2.022 | 1.00 | 34.48 | C |
| ATOM | 2002 | CB | THR | A | 257 | 23.191 | −0.083 | 2.511 | 1.00 | 37.17 | C |
| ATOM | 2003 | OG1 | THR | A | 257 | 22.728 | −1.175 | 1.712 | 1.00 | 37.28 | O |
| ATOM | 2004 | CG2 | THR | A | 257 | 22.298 | 1.143 | 2.478 | 1.00 | 36.91 | C |
| ATOM | 2005 | C | THR | A | 257 | 25.580 | −0.905 | 2.256 | 1.00 | 33.97 | C |
| ATOM | 2006 | O | THR | A | 257 | 25.750 | −1.262 | 3.438 | 1.00 | 27.14 | O |
| ATOM | 2007 | N | ASP | A | 258 | 26.244 | −1.420 | 1.215 | 1.00 | 36.02 | N |
| ATOM | 2008 | CA | ASP | A | 258 | 27.455 | −2.248 | 1.429 | 1.00 | 40.95 | C |
| ATOM | 2009 | CB | ASP | A | 258 | 27.083 | −3.674 | 1.816 | 1.00 | 45.69 | C |
| ATOM | 2010 | CG | ASP | A | 258 | 28.195 | −4.359 | 2.586 | 1.00 | 48.54 | C |
| ATOM | 2011 | OD1 | ASP | A | 258 | 29.194 | −3.658 | 2.889 | 1.00 | 50.57 | O |
| ATOM | 2012 | OD2 | ASP | A | 258 | 28.060 | −5.581 | 2.864 | 1.00 | 49.97 | O |
| ATOM | 2013 | C | ASP | A | 258 | 28.415 | −2.184 | 0.234 | 1.00 | 42.42 | C |
| ATOM | 2014 | O | ASP | A | 258 | 27.978 | −1.931 | −0.917 | 1.00 | 39.71 | O |
| ATOM | 2015 | N | VAL | A | 259 | 29.712 | −2.304 | 0.537 | 1.00 | 43.12 | N |
| ATOM | 2016 | CA | VAL | A | 259 | 30.815 | −2.448 | −0.458 | 1.00 | 43.47 | C |
| ATOM | 2017 | CB | VAL | A | 259 | 31.667 | −1.179 | −0.616 | 1.00 | 46.58 | C |
| ATOM | 2018 | CG1 | VAL | A | 259 | 32.577 | −1.255 | −1.828 | 1.00 | 44.78 | C |
| ATOM | 2019 | CG2 | VAL | A | 259 | 30.808 | 0.064 | −0.715 | 1.00 | 49.64 | C |
| ATOM | 2020 | C | VAL | A | 259 | 31.641 | −3.610 | 0.053 | 1.00 | 48.18 | C |
| ATOM | 2021 | O | VAL | A | 259 | 31.900 | −3.649 | 1.277 | 1.00 | 48.64 | O |
| ATOM | 2022 | N | ARG | A | 260 | 31.939 | −4.577 | −0.806 | 1.00 | 51.26 | N |
| ATOM | 2023 | CA | ARG | A | 260 | 32.403 | −5.888 | −0.297 | 1.00 | 56.33 | C |
| ATOM | 2024 | CB | ARG | A | 260 | 31.206 | −6.611 | 0.319 | 1.00 | 57.58 | C |
| ATOM | 2025 | CG | ARG | A | 260 | 31.558 | −7.875 | 1.079 | 1.00 | 62.02 | C |
| ATOM | 2026 | CD | ARG | A | 260 | 30.311 | −8.403 | 1.746 | 1.00 | 61.01 | C |
| ATOM | 2027 | NE | ARG | A | 260 | 30.007 | −7.728 | 2.996 | 1.00 | 52.15 | N |
| ATOM | 2028 | CZ | ARG | A | 260 | 30.123 | −8.281 | 4.192 | 1.00 | 57.91 | C |
| ATOM | 2029 | NH1 | ARG | A | 260 | 30.558 | −9.520 | 4.331 | 1.00 | 62.73 | N |
| ATOM | 2030 | NH2 | ARG | A | 260 | 29.790 | −7.598 | 5.268 | 1.00 | 64.01 | N |
| ATOM | 2031 | C | ARG | A | 260 | 33.017 | −6.713 | −1.422 | 1.00 | 59.28 | C |
| ATOM | 2032 | O | ARG | A | 260 | 32.340 | −6.901 | −2.456 | 1.00 | 51.49 | O |
| ATOM | 2033 | N | LYS | A | 261 | 34.254 | −7.175 | −1.206 | 1.00 | 63.49 | N |
| ATOM | 2034 | CA | LYS | A | 261 | 34.829 | −8.349 | −1.911 | 1.00 | 64.14 | C |
| ATOM | 2035 | CB | LYS | A | 261 | 36.354 | −8.371 | −1.774 | 1.00 | 69.56 | C |
| ATOM | 2036 | CG | LYS | A | 261 | 37.059 | −7.080 | −2.178 | 1.00 | 74.04 | C |
| ATOM | 2037 | CD | LYS | A | 261 | 38.564 | −7.223 | −2.358 | 1.00 | 74.85 | C |
| ATOM | 2038 | CE | LYS | A | 261 | 38.966 | −7.600 | −3.770 | 1.00 | 74.99 | C |
| ATOM | 2039 | NZ | LYS | A | 261 | 38.917 | −6.433 | −4.685 | 1.00 | 73.73 | N |
| ATOM | 2040 | C | LYS | A | 261 | 34.169 | −9.587 | −1.296 | 1.00 | 61.37 | C |
| ATOM | 2041 | O | LYS | A | 261 | 34.107 | −9.660 | −0.039 | 1.00 | 58.89 | O |
| ATOM | 2042 | N | VAL | A | 262 | 33.613 | −10.475 | −2.125 | 1.00 | 58.83 | N |
| ATOM | 2043 | CA | VAL | A | 262 | 33.045 | −11.774 | −1.650 | 1.00 | 63.11 | C |
| ATOM | 2044 | CB | VAL | A | 262 | 31.504 | −11.850 | −1.763 | 1.00 | 64.35 | C |
| ATOM | 2045 | CG1 | VAL | A | 262 | 30.839 | −10.772 | −0.914 | 1.00 | 61.82 | C |
| ATOM | 2046 | CG2 | VAL | A | 262 | 30.998 | −11.805 | −3.201 | 1.00 | 58.80 | C |
| ATOM | 2047 | C | VAL | A | 262 | 33.744 | −12.914 | −2.397 | 1.00 | 65.51 | C |
| ATOM | 2048 | O | VAL | A | 262 | 34.010 | −12.771 | −3.624 | 1.00 | 57.21 | O |
| ATOM | 2049 | N | ARG | A | 263 | 34.060 | −13.978 | −1.651 | 1.00 | 73.12 | N |
| ATOM | 2050 | CA | ARG | A | 263 | 34.639 | −15.247 | −2.161 | 1.00 | 75.98 | C |
| ATOM | 2051 | CB | ARG | A | 263 | 35.717 | −15.756 | −1.198 | 1.00 | 78.23 | C |
| ATOM | 2052 | C | ARG | A | 263 | 33.501 | −16.261 | −2.329 | 1.00 | 76.58 | C |
| ATOM | 2053 | O | ARG | A | 263 | 32.679 | −16.387 | −1.395 | 1.00 | 76.17 | O |
| ATOM | 2054 | N | LEU | A | 264 | 33.464 | −16.961 | −3.464 | 1.00 | 79.90 | N |
| ATOM | 2055 | CA | LEU | A | 264 | 32.475 | −18.037 | −3.758 | 1.00 | 86.92 | C |
| ATOM | 2056 | CB | LEU | A | 264 | 32.044 | −17.948 | −5.226 | 1.00 | 93.04 | C |
| ATOM | 2057 | CG | LEU | A | 264 | 31.665 | −16.558 | −5.736 | 1.00 | 96.16 | C |
| ATOM | 2058 | CD1 | LEU | A | 264 | 32.880 | −15.841 | −6.308 | 1.00 | 100.96 | C |
| ATOM | 2059 | CD2 | LEU | A | 264 | 30.565 | −16.648 | −6.785 | 1.00 | 96.58 | C |
| ATOM | 2060 | C | LEU | A | 264 | 33.092 | −19.415 | −3.470 | 1.00 | 91.14 | C |
| ATOM | 2061 | O | LEU | A | 264 | 34.291 | −19.601 | −3.789 | 1.00 | 78.14 | O |
| ATOM | 2062 | N | GLY | A | 265 | 32.290 | −20.343 | −2.918 | 1.00 | 104.68 | N |
| ATOM | 2063 | CA | GLY | A | 265 | 32.645 | −21.756 | −2.645 | 1.00 | 110.58 | C |
| ATOM | 2064 | C | GLY | A | 265 | 32.995 | −22.522 | −3.915 | 1.00 | 119.14 | C |
| ATOM | 2065 | O | GLY | A | 265 | 32.794 | −21.963 | −5.006 | 1.00 | 116.28 | O |
| ATOM | 2066 | N | HIS | A | 266 | 33.526 | −23.746 | −3.790 | 1.00 | 129.88 | N |
| ATOM | 2067 | CA | HIS | A | 266 | 33.988 | −24.579 | −4.937 | 1.00 | 128.18 | C |
| ATOM | 2068 | CB | HIS | A | 266 | 34.830 | −25.781 | −4.481 | 1.00 | 129.17 | C |
| ATOM | 2069 | CG | HIS | A | 266 | 35.422 | −26.556 | −5.616 | 1.00 | 131.14 | C |
| ATOM | 2070 | ND1 | HIS | A | 266 | 36.563 | −26.137 | −6.289 | 1.00 | 124.55 | N |
| ATOM | 2071 | CE1 | HIS | A | 266 | 36.852 | −26.998 | −7.243 | 1.00 | 119.56 | C |
| ATOM | 2072 | NE2 | HIS | A | 266 | 35.925 | −27.970 | −7.217 | 1.00 | 124.79 | N |
| ATOM | 2073 | CD2 | HIS | A | 266 | 35.032 | −27.704 | −6.213 | 1.00 | 122.99 | C |
| ATOM | 2074 | C | HIS | A | 266 | 32.775 | −25.026 | −5.757 | 1.00 | 125.55 | C |
| ATOM | 2075 | O | HIS | A | 266 | 32.890 | −25.076 | −6.996 | 1.00 | 122.37 | O |
| ATOM | 2076 | N | SER | A | 267 | 31.656 | −25.311 | −5.085 | 1.00 | 119.38 | N |
| ATOM | 2077 | CA | SER | A | 267 | 30.376 | −25.748 | −5.703 | 1.00 | 117.04 | C |
| ATOM | 2078 | CB | SER | A | 267 | 29.318 | −25.987 | −4.646 | 1.00 | 113.06 | C |
| ATOM | 2079 | OG | SER | A | 267 | 29.244 | −24.898 | −3.734 | 1.00 | 105.48 | O |
| ATOM | 2080 | C | SER | A | 267 | 29.905 | −24.730 | −6.755 | 1.00 | 118.06 | C |
| ATOM | 2081 | O | SER | A | 267 | 29.059 | −25.117 | −7.581 | 1.00 | 104.07 | O |
| ATOM | 2082 | N | LEU | A | 268 | 30.447 | −23.500 | −6.747 | 1.00 | 119.10 | N |
| ATOM | 2083 | CA | LEU | A | 268 | 29.932 | −22.339 | −7.529 | 1.00 | 112.86 | C |
| ATOM | 2084 | CB | LEU | A | 268 | 29.766 | −21.132 | −6.599 | 1.00 | 108.53 | C |
| ATOM | 2085 | CG | LEU | A | 268 | 28.731 | −21.275 | −5.482 | 1.00 | 112.77 | C |
| ATOM | 2086 | CD1 | LEU | A | 268 | 29.353 | −21.851 | −4.217 | 1.00 | 119.36 | C |
| ATOM | 2087 | CD2 | LEU | A | 268 | 28.087 | −19.931 | −5.175 | 1.00 | 116.26 | C |
| ATOM | 2088 | C | LEU | A | 268 | 30.852 | −21.977 | −8.705 | 1.00 | 107.88 | C |
| ATOM | 2089 | O | LEU | A | 268 | 30.387 | −21.220 | −9.571 | 1.00 | 113.10 | O |
| ATOM | 2090 | N | LEU | A | 269 | 32.095 | −22.467 | −8.758 | 1.00 | 113.56 | N |
| ATOM | 2091 | CA | LEU | A | 269 | 33.026 | −22.150 | −9.880 | 1.00 | 110.86 | C |
| ATOM | 2092 | CB | LEU | A | 269 | 34.444 | −22.607 | −9.520 | 1.00 | 114.18 | C |
| ATOM | 2093 | CG | LEU | A | 269 | 35.576 | −21.873 | −10.240 | 1.00 | 119.91 | C |
| ATOM | 2094 | CD1 | LEU | A | 269 | 36.653 | −21.428 | −9.257 | 1.00 | 117.54 | C |
| ATOM | 2095 | CD2 | LEU | A | 269 | 36.175 | −22.738 | −11.344 | 1.00 | 116.19 | C |
| ATOM | 2096 | C | LEU | A | 269 | 32.491 | −22.823 | −11.152 | 1.00 | 106.58 | C |
| ATOM | 2097 | O | LEU | A | 269 | 31.483 | −23.547 | −11.058 | 1.00 | 104.40 | O |
| ATOM | 2098 | N | ALA | A | 270 | 33.080 | −22.532 | −12.311 | 1.00 | 113.80 | N |
| ATOM | 2099 | CA | ALA | A | 270 | 32.619 | −23.022 | −13.635 | 1.00 | 128.09 | C |
| ATOM | 2100 | CB | ALA | A | 270 | 32.328 | −24.507 | −13.587 | 1.00 | 124.30 | C |
| ATOM | 2101 | C | ALA | A | 270 | 31.390 | −22.215 | −14.091 | 1.00 | 132.27 | C |
| ATOM | 2102 | O | ALA | A | 270 | 31.391 | −21.745 | −15.271 | 1.00 | 120.64 | O |
| ATOM | 2103 | N | PHE | A | 271 | 30.388 | −22.049 | −13.210 | 1.00 | 123.37 | N |
| ATOM | 2104 | CA | PHE | A | 271 | 29.195 | −21.192 | −13.455 | 1.00 | 114.54 | C |
| ATOM | 2105 | CB | PHE | A | 271 | 28.036 | −21.504 | −12.503 | 1.00 | 104.93 | C |
| ATOM | 2106 | CG | PHE | A | 271 | 26.798 | −20.693 | −12.805 | 1.00 | 105.47 | C |
| ATOM | 2107 | CD1 | PHE | A | 271 | 25.863 | −21.138 | −13.729 | 1.00 | 100.90 | C |
| ATOM | 2108 | CE1 | PHE | A | 271 | 24.744 | −20.375 | −14.029 | 1.00 | 101.35 | C |
| ATOM | 2109 | CZ | PHE | A | 271 | 24.546 | −19.160 | −13.413 | 1.00 | 97.60 | C |
| ATOM | 2110 | CD2 | PHE | A | 271 | 26.590 | −19.457 | −12.203 | 1.00 | 103.22 | C |
| ATOM | 2111 | CE2 | PHE | A | 271 | 25.469 | −18.697 | −12.505 | 1.00 | 96.39 | C |
| ATOM | 2112 | C | PHE | A | 271 | 29.585 | −19.707 | −13.372 | 1.00 | 114.90 | C |
| ATOM | 2113 | O | PHE | A | 271 | 29.212 | −18.964 | −14.294 | 1.00 | 117.33 | O |
| ATOM | 2114 | N | PHE | A | 272 | 30.298 | −19.283 | −12.320 | 1.00 | 113.75 | N |
| ATOM | 2115 | CA | PHE | A | 272 | 30.739 | −17.872 | −12.124 | 1.00 | 116.02 | C |
| ATOM | 2116 | CB | PHE | A | 272 | 30.671 | −17.478 | −10.649 | 1.00 | 110.72 | C |
| ATOM | 2117 | CG | PHE | A | 272 | 29.263 | −17.292 | −10.152 | 1.00 | 101.83 | C |
| ATOM | 2118 | CD1 | PHE | A | 272 | 28.528 | −16.173 | −10.510 | 1.00 | 91.73 | C |
| ATOM | 2119 | CE1 | PHE | A | 272 | 27.230 | −16.007 | −10.053 | 1.00 | 90.81 | C |
| ATOM | 2120 | CZ | PHE | A | 272 | 26.651 | −16.966 | −9.252 | 1.00 | 92.69 | C |
| ATOM | 2121 | CD2 | PHE | A | 272 | 28.664 | −18.253 | −9.353 | 1.00 | 100.91 | C |
| ATOM | 2122 | CE2 | PHE | A | 272 | 27.364 | −18.088 | −8.901 | 1.00 | 98.26 | C |
| ATOM | 2123 | C | PHE | A | 272 | 32.148 | −17.648 | −12.686 | 1.00 | 123.60 | C |
| ATOM | 2124 | O | PHE | A | 272 | 32.458 | −16.494 | −13.046 | 1.00 | 126.27 | O |
| ATOM | 2125 | N | LYS | A | 273 | 32.969 | −18.704 | −12.741 | 1.00 | 131.51 | N |
| ATOM | 2126 | CA | LYS | A | 273 | 34.333 | −18.695 | −13.337 | 1.00 | 133.32 | C |
| ATOM | 2127 | CB | LYS | A | 273 | 34.294 | −18.290 | −14.818 | 1.00 | 139.83 | C |
| ATOM | 2128 | CG | LYS | A | 273 | 33.334 | −19.066 | −15.711 | 1.00 | 140.95 | C |
| ATOM | 2129 | CD | LYS | A | 273 | 33.189 | −18.464 | −17.099 | 1.00 | 140.26 | C |
| ATOM | 2130 | CE | LYS | A | 273 | 32.054 | −19.073 | −17.894 | 1.00 | 137.26 | C |
| ATOM | 2131 | NZ | LYS | A | 273 | 32.253 | −20.525 | −18.104 | 1.00 | 140.91 | N |
| ATOM | 2132 | C | LYS | A | 273 | 35.229 | −17.722 | −12.560 | 1.00 | 125.37 | C |
| ATOM | 2133 | O | LYS | A | 273 | 36.144 | −17.160 | −13.172 | 1.00 | 124.42 | O |
| ATOM | 2134 | N | SER | A | 274 | 34.979 | −17.516 | −11.266 | 1.00 | 115.76 | N |
| ATOM | 2135 | CA | SER | A | 274 | 35.784 | −16.603 | −10.416 | 1.00 | 111.36 | C |
| ATOM | 2136 | CB | SER | A | 274 | 35.420 | −15.166 | −10.665 | 1.00 | 110.83 | C |
| ATOM | 2137 | OG | SER | A | 274 | 36.277 | −14.298 | −9.939 | 1.00 | 114.42 | O |
| ATOM | 2138 | C | SER | A | 274 | 35.612 | −16.976 | −8.944 | 1.00 | 109.17 | C |
| ATOM | 2139 | O | SER | A | 274 | 34.599 | −17.609 | −8.614 | 1.00 | 107.95 | O |
| ATOM | 2140 | N | GLU | A | 275 | 36.583 | −16.598 | −8.112 | 1.00 | 106.83 | N |
| ATOM | 2141 | CA | GLU | A | 275 | 36.586 | −16.855 | −6.649 | 1.00 | 110.92 | C |
| ATOM | 2142 | CB | GLU | A | 275 | 37.930 | −17.465 | −6.235 | 1.00 | 119.98 | C |
| ATOM | 2143 | CG | GLU | A | 275 | 37.914 | −18.145 | −4.871 | 1.00 | 122.75 | C |
| ATOM | 2144 | CD | GLU | A | 275 | 38.923 | −19.272 | −4.699 | 1.00 | 121.36 | C |
| ATOM | 2145 | OE1 | GLU | A | 275 | 39.059 | −20.084 | −5.636 | 1.00 | 116.35 | O |
| ATOM | 2146 | OE2 | GLU | A | 275 | 39.571 | −19.341 | −3.629 | 1.00 | 114.46 | O |
| ATOM | 2147 | C | GLU | A | 275 | 36.266 | −15.553 | −5.900 | 1.00 | 107.15 | C |
| ATOM | 2148 | O | GLU | A | 275 | 35.720 | −15.657 | −4.793 | 1.00 | 101.96 | O |
| ATOM | 2149 | N | LYS | A | 276 | 36.597 | −14.385 | −6.468 | 1.00 | 100.13 | N |
| ATOM | 2150 | CA | LYS | A | 276 | 36.338 | −13.045 | −5.868 | 1.00 | 95.29 | C |
| ATOM | 2151 | CB | LYS | A | 276 | 37.642 | −12.276 | −5.625 | 1.00 | 105.45 | C |
| ATOM | 2152 | CG | LYS | A | 276 | 38.468 | −12.709 | −4.416 | 1.00 | 112.17 | C |
| ATOM | 2153 | CD | LYS | A | 276 | 39.953 | −12.342 | −4.508 | 1.00 | 107.41 | C |
| ATOM | 2154 | CE | LYS | A | 276 | 40.535 | −11.771 | −3.227 | 1.00 | 106.99 | C |
| ATOM | 2155 | NZ | LYS | A | 276 | 40.747 | −12.806 | −2.187 | 1.00 | 102.67 | N |
| ATOM | 2156 | C | LYS | A | 276 | 35.457 | −12.220 | −6.812 | 1.00 | 85.04 | C |
| ATOM | 2157 | O | LYS | A | 276 | 35.813 | −12.092 | −8.003 | 1.00 | 75.30 | O |
| ATOM | 2158 | N | LEU | A | 277 | 34.359 | −11.672 | −6.291 | 1.00 | 74.50 | N |
| ATOM | 2159 | CA | LEU | A | 277 | 33.495 | −10.692 | −7.001 | 1.00 | 66.82 | C |
| ATOM | 2160 | CB | LEU | A | 277 | 32.082 | −11.260 | −7.117 | 1.00 | 63.79 | C |
| ATOM | 2161 | CG | LEU | A | 277 | 31.867 | −12.257 | −8.251 | 1.00 | 65.67 | C |
| ATOM | 2162 | CD1 | LEU | A | 277 | 30.516 | −12.943 | −8.100 | 1.00 | 67.20 | C |
| ATOM | 2163 | CD2 | LEU | A | 277 | 31.970 | −11.584 | −9.614 | 1.00 | 63.09 | C |
| ATOM | 2164 | C | LEU | A | 277 | 33.484 | −9.370 | −6.225 | 1.00 | 62.64 | C |
| ATOM | 2165 | O | LEU | A | 277 | 33.366 | −9.421 | −4.973 | 1.00 | 49.12 | O |
| ATOM | 2166 | N | ASP | A | 278 | 33.612 | −8.250 | −6.948 | 1.00 | 61.52 | N |
| ATOM | 2167 | CA | ASP | A | 278 | 33.526 | −6.874 | −6.392 | 1.00 | 60.78 | C |
| ATOM | 2168 | CB | ASP | A | 278 | 34.440 | −5.930 | −7.169 | 1.00 | 61.08 | C |
| ATOM | 2169 | CG | ASP | A | 278 | 35.903 | −6.155 | −6.833 | 1.00 | 59.64 | C |
| ATOM | 2170 | OD1 | ASP | A | 278 | 36.178 | −6.912 | −5.874 | 1.00 | 60.71 | O |
| ATOM | 2171 | OD2 | ASP | A | 278 | 36.750 | −5.567 | −7.516 | 1.00 | 62.04 | O |
| ATOM | 2172 | C | ASP | A | 278 | 32.053 | −6.458 | −6.384 | 1.00 | 60.93 | C |
| ATOM | 2173 | O | ASP | A | 278 | 31.502 | −6.190 | −7.472 | 1.00 | 59.91 | O |
| ATOM | 2174 | N | MET | A | 279 | 31.439 | −6.460 | −5.195 | 1.00 | 55.17 | N |
| ATOM | 2175 | CA | MET | A | 279 | 29.968 | −6.365 | −5.005 | 1.00 | 53.48 | C |
| ATOM | 2176 | CB | MET | A | 279 | 29.428 | −7.505 | −4.135 | 1.00 | 53.82 | C |
| ATOM | 2177 | CG | MET | A | 279 | 29.683 | −8.875 | −4.706 | 1.00 | 55.15 | C |
| ATOM | 2178 | SD | MET | A | 279 | 28.864 | −9.110 | −6.277 | 1.00 | 60.33 | S |
| ATOM | 2179 | CE | MET | A | 279 | 27.193 | −9.370 | −5.686 | 1.00 | 62.12 | C |
| ATOM | 2180 | C | MET | A | 279 | 29.628 | −5.042 | −4.320 | 1.00 | 51.90 | C |
| ATOM | 2181 | O | MET | A | 279 | 30.422 | −4.568 | −3.472 | 1.00 | 54.21 | O |
| ATOM | 2182 | N | ILE | A | 280 | 28.463 | −4.492 | −4.644 | 1.00 | 47.86 | N |
| ATOM | 2183 | CA | ILE | A | 280 | 27.888 | −3.366 | −3.860 | 1.00 | 45.98 | C |
| ATOM | 2184 | CB | ILE | A | 280 | 28.277 | −2.017 | −4.485 | 1.00 | 50.72 | C |
| ATOM | 2185 | CG1 | ILE | A | 280 | 27.087 | −1.075 | −4.589 | 1.00 | 49.40 | C |
| ATOM | 2186 | CG2 | ILE | A | 280 | 28.969 | −2.201 | −5.825 | 1.00 | 52.68 | C |
| ATOM | 2187 | CD1 | ILE | A | 280 | 27.415 | 0.184 | −5.334 | 1.00 | 58.69 | C |
| ATOM | 2188 | C | ILE | A | 280 | 26.383 | −3.582 | −3.702 | 1.00 | 41.30 | C |
| ATOM | 2189 | O | ILE | A | 280 | 25.747 | −4.119 | −4.609 | 1.00 | 36.61 | O |
| ATOM | 2190 | N | ARG | A | 281 | 25.888 | −3.265 | −2.515 | 1.00 | 39.27 | N |
| ATOM | 2191 | CA | ARG | A | 281 | 24.464 | −3.285 | −2.156 | 1.00 | 39.76 | C |
| ATOM | 2192 | CB | ARG | A | 281 | 24.289 | −3.951 | −0.802 | 1.00 | 42.17 | C |
| ATOM | 2193 | CG | ARG | A | 281 | 22.850 | −3.924 | −0.321 | 1.00 | 40.00 | C |
| ATOM | 2194 | CD | ARG | A | 281 | 22.712 | −4.714 | 0.952 | 1.00 | 40.83 | C |
| ATOM | 2195 | NE | ARG | A | 281 | 23.228 | −4.000 | 2.098 | 1.00 | 41.23 | N |
| ATOM | 2196 | CZ | ARG | A | 281 | 23.582 | −4.561 | 3.246 | 1.00 | 45.38 | C |
| ATOM | 2197 | NH1 | ARG | A | 281 | 23.530 | −5.874 | 3.403 | 1.00 | 46.92 | N |
| ATOM | 2198 | NH2 | ARG | A | 281 | 24.021 | −3.800 | 4.230 | 1.00 | 44.11 | N |
| ATOM | 2199 | C | ARG | A | 281 | 23.941 | −1.851 | −2.079 | 1.00 | 42.88 | C |
| ATOM | 2200 | O | ARG | A | 281 | 24.646 | −0.997 | −1.494 | 1.00 | 42.96 | O |
| ATOM | 2201 | N | LEU | A | 282 | 22.744 | −1.620 | −2.627 | 1.00 | 37.90 | N |
| ATOM | 2202 | CA | LEU | A | 282 | 22.107 | −0.287 | −2.709 | 1.00 | 38.07 | C |
| ATOM | 2203 | CB | LEU | A | 282 | 22.111 | 0.166 | −4.166 | 1.00 | 39.47 | C |
| ATOM | 2204 | CG | LEU | A | 282 | 23.486 | 0.224 | −4.832 | 1.00 | 44.68 | C |
| ATOM | 2205 | CD1 | LEU | A | 282 | 23.655 | −0.908 | −5.835 | 1.00 | 45.61 | C |
| ATOM | 2206 | CD2 | LEU | A | 282 | 23.713 | 1.578 | −5.514 | 1.00 | 48.05 | C |
| ATOM | 2207 | C | LEU | A | 282 | 20.676 | −0.343 | −2.168 | 1.00 | 39.82 | C |
| ATOM | 2208 | O | LEU | A | 282 | 20.055 | −1.414 | −2.242 | 1.00 | 39.02 | O |
| ATOM | 2209 | N | ARG | A | 283 | 20.171 | 0.784 | −1.664 | 1.00 | 40.07 | N |
| ATOM | 2210 | CA | ARG | A | 283 | 18.749 | 0.974 | −1.280 | 1.00 | 42.22 | C |
| ATOM | 2211 | CB | ARG | A | 283 | 18.611 | 1.637 | 0.094 | 1.00 | 45.82 | C |
| ATOM | 2212 | CG | ARG | A | 283 | 17.186 | 1.974 | 0.535 | 1.00 | 47.37 | C |
| ATOM | 2213 | CD | ARG | A | 283 | 16.691 | 1.138 | 1.722 | 1.00 | 55.81 | C |
| ATOM | 2214 | NE | ARG | A | 283 | 15.500 | 1.660 | 2.380 | 1.00 | 60.19 | N |
| ATOM | 2215 | CZ | ARG | A | 283 | 14.272 | 1.759 | 1.833 | 1.00 | 68.38 | C |
| ATOM | 2216 | NH1 | ARG | A | 283 | 13.268 | 2.270 | 2.530 | 1.00 | 69.06 | N |
| ATOM | 2217 | NH2 | ARG | A | 283 | 14.030 | 1.367 | 0.594 | 1.00 | 67.61 | N |
| ATOM | 2218 | C | ARG | A | 283 | 18.103 | 1.845 | −2.348 | 1.00 | 40.85 | C |
| ATOM | 2219 | O | ARG | A | 283 | 18.708 | 2.860 | −2.723 | 1.00 | 42.58 | O |
| ATOM | 2220 | N | ASN | A | 284 | 16.934 | 1.436 | −2.836 | 1.00 | 39.79 | N |
| ATOM | 2221 | CA | ASN | A | 284 | 16.004 | 2.342 | −3.549 | 1.00 | 37.34 | C |
| ATOM | 2222 | CB | ASN | A | 284 | 15.050 | 1.568 | −4.435 | 1.00 | 38.14 | C |
| ATOM | 2223 | CG | ASN | A | 284 | 14.072 | 2.502 | −5.108 | 1.00 | 38.82 | C |
| ATOM | 2224 | OD1 | ASN | A | 284 | 14.234 | 3.722 | −5.052 | 1.00 | 36.44 | O |
| ATOM | 2225 | ND2 | ASN | A | 284 | 13.072 | 1.927 | −5.753 | 1.00 | 40.54 | N |
| ATOM | 2226 | C | ASN | A | 284 | 15.228 | 3.166 | −2.521 | 1.00 | 39.51 | C |
| ATOM | 2227 | O | ASN | A | 284 | 14.432 | 2.599 | −1.786 | 1.00 | 40.88 | O |
| ATOM | 2228 | N | PRO | A | 285 | 15.430 | 4.506 | −2.415 | 1.00 | 36.62 | N |
| ATOM | 2229 | CA | PRO | A | 285 | 14.678 | 5.330 | −1.462 | 1.00 | 35.27 | C |
| ATOM | 2230 | CB | PRO | A | 285 | 15.244 | 6.753 | −1.623 | 1.00 | 35.19 | C |
| ATOM | 2231 | CG | PRO | A | 285 | 16.510 | 6.591 | −2.442 | 1.00 | 37.84 | C |
| ATOM | 2232 | CD | PRO | A | 285 | 16.353 | 5.303 | −3.233 | 1.00 | 36.89 | C |
| ATOM | 2233 | C | PRO | A | 285 | 13.161 | 5.376 | −1.753 | 1.00 | 33.62 | C |
| ATOM | 2234 | O | PRO | A | 285 | 12.410 | 5.498 | −0.806 | 1.00 | 31.58 | O |
| ATOM | 2235 | N | TRP | A | 286 | 12.771 | 5.237 | −3.028 | 1.00 | 31.65 | N |
| ATOM | 2236 | CA | TRP | A | 286 | 11.383 | 5.362 | −3.547 | 1.00 | 37.22 | C |
| ATOM | 2237 | CB | TRP | A | 286 | 11.411 | 5.730 | −5.034 | 1.00 | 37.54 | C |
| ATOM | 2238 | CG | TRP | A | 286 | 11.702 | 7.176 | −5.271 | 1.00 | 39.05 | C |
| ATOM | 2239 | CD1 | TRP | A | 286 | 10.793 | 8.192 | −5.361 | 1.00 | 43.71 | C |
| ATOM | 2240 | NE1 | TRP | A | 286 | 11.435 | 9.387 | −5.551 | 1.00 | 45.35 | N |
| ATOM | 2241 | CE2 | TRP | A | 286 | 12.787 | 9.171 | −5.571 | 1.00 | 43.90 | C |
| ATOM | 2242 | CD2 | TRP | A | 286 | 12.998 | 7.786 | −5.386 | 1.00 | 42.07 | C |
| ATOM | 2243 | CE3 | TRP | A | 286 | 14.309 | 7.299 | −5.383 | 1.00 | 37.92 | C |
| ATOM | 2244 | CZ3 | TRP | A | 286 | 15.343 | 8.187 | −5.555 | 1.00 | 41.55 | C |
| ATOM | 2245 | CH2 | TRP | A | 286 | 15.114 | 9.553 | −5.727 | 1.00 | 40.94 | C |
| ATOM | 2246 | CZ2 | TRP | A | 286 | 13.839 | 10.067 | −5.748 | 1.00 | 43.76 | C |
| ATOM | 2247 | C | TRP | A | 286 | 10.542 | 4.097 | −3.289 | 1.00 | 42.85 | C |
| ATOM | 2248 | O | TRP | A | 286 | 9.310 | 4.160 | −3.446 | 1.00 | 45.73 | O |
| ATOM | 2249 | N | GLY | A | 287 | 11.158 | 2.996 | −2.870 | 1.00 | 44.63 | N |
| ATOM | 2250 | CA | GLY | A | 287 | 10.431 | 1.811 | −2.392 | 1.00 | 45.20 | C |
| ATOM | 2251 | C | GLY | A | 287 | 11.150 | 0.531 | −2.751 | 1.00 | 41.02 | C |
| ATOM | 2252 | O | GLY | A | 287 | 12.324 | 0.405 | −2.411 | 1.00 | 38.22 | O |
| ATOM | 2253 | N | GLU | A | 288 | 10.447 | −0.376 | −3.410 | 1.00 | 42.86 | N |
| ATOM | 2254 | CA | GLU | A | 288 | 10.909 | −1.751 | −3.699 | 1.00 | 39.74 | C |
| ATOM | 2255 | CB | GLU | A | 288 | 9.739 | −2.581 | −4.200 | 1.00 | 39.16 | C |
| ATOM | 2256 | CG | GLU | A | 288 | 10.164 | −3.908 | −4.773 | 1.00 | 49.50 | C |
| ATOM | 2257 | CD | GLU | A | 288 | 9.049 | −4.924 | −4.915 | 1.00 | 49.59 | C |
| ATOM | 2258 | OE1 | GLU | A | 288 | 7.943 | −4.643 | −4.425 | 1.00 | 61.28 | O |
| ATOM | 2259 | OE2 | GLU | A | 288 | 9.293 | −5.991 | −5.509 | 1.00 | 58.61 | O |
| ATOM | 2260 | C | GLU | A | 288 | 12.033 | −1.699 | −4.728 | 1.00 | 38.18 | C |
| ATOM | 2261 | O | GLU | A | 288 | 11.923 | −0.886 | −5.651 | 1.00 | 36.29 | O |
| ATOM | 2262 | N | ARG | A | 289 | 13.064 | −2.539 | −4.549 | 1.00 | 33.11 | N |
| ATOM | 2263 | CA | ARG | A | 289 | 14.174 | −2.676 | −5.513 | 1.00 | 37.83 | C |
| ATOM | 2264 | CB | ARG | A | 289 | 15.126 | −3.822 | −5.146 | 1.00 | 40.82 | C |
| ATOM | 2265 | CG | ARG | A | 289 | 14.512 | −5.212 | −5.097 | 1.00 | 42.52 | C |
| ATOM | 2266 | CD | ARG | A | 289 | 14.693 | −5.959 | −6.399 | 1.00 | 48.36 | C |
| ATOM | 2267 | NE | ARG | A | 289 | 13.958 | −7.220 | −6.387 | 1.00 | 56.88 | N |
| ATOM | 2268 | CZ | ARG | A | 289 | 12.637 | −7.325 | −6.535 | 1.00 | 58.78 | C |
| ATOM | 2269 | NH1 | ARG | A | 289 | 11.878 | −6.247 | −6.694 | 1.00 | 50.64 | N |
| ATOM | 2270 | NH2 | ARG | A | 289 | 12.076 | −8.518 | −6.504 | 1.00 | 58.67 | N |
| ATOM | 2271 | C | ARG | A | 289 | 13.570 | −2.857 | −6.904 | 1.00 | 41.17 | C |
| ATOM | 2272 | O | ARG | A | 289 | 12.509 | −3.492 | −7.017 | 1.00 | 44.42 | O |
| ATOM | 2273 | N | GLU | A | 290 | 14.224 | −2.310 | −7.923 | 1.00 | 41.63 | N |
| ATOM | 2274 | CA | GLU | A | 290 | 13.724 | −2.353 | −9.321 | 1.00 | 44.99 | C |
| ATOM | 2275 | CB | GLU | A | 290 | 13.599 | −0.922 | −9.850 | 1.00 | 47.92 | C |
| ATOM | 2276 | CG | GLU | A | 290 | 12.973 | 0.056 | −8.866 | 1.00 | 46.65 | C |
| ATOM | 2277 | CD | GLU | A | 290 | 13.506 | 1.476 | −8.982 | 1.00 | 47.37 | C |
| ATOM | 2278 | OE1 | GLU | A | 290 | 14.741 | 1.624 | −9.045 | 1.00 | 49.72 | O |
| ATOM | 2279 | OE2 | GLU | A | 290 | 12.695 | 2.426 | −9.017 | 1.00 | 47.14 | O |
| ATOM | 2280 | C | GLU | A | 290 | 14.672 | −3.177 | 10.193 | 1.00 | 43.67 | C |
| ATOM | 2281 | O | GLU | A | 290 | 14.240 | −3.640 | −11.254 | 1.00 | 55.24 | O |
| ATOM | 2282 | N | TRP | A | 291 | 15.923 | −3.311 | −9.754 | 1.00 | 46.54 | N |
| ATOM | 2283 | CA | TRP | A | 291 | 17.074 | −3.942 | −10.458 | 1.00 | 44.81 | C |
| ATOM | 2284 | CB | TRP | A | 291 | 18.269 | −4.005 | −9.502 | 1.00 | 41.08 | C |
| ATOM | 2285 | CG | TRP | A | 291 | 19.445 | −4.798 | −9.984 | 1.00 | 39.50 | C |
| ATOM | 2286 | CD1 | TRP | A | 291 | 20.040 | −5.848 | −9.356 | 1.00 | 34.62 | C |
| ATOM | 2287 | NE1 | TRP | A | 291 | 21.097 | −6.301 | −10.089 | 1.00 | 36.30 | N |
| ATOM | 2288 | CE2 | TRP | A | 291 | 21.217 | −5.545 | −11.217 | 1.00 | 37.93 | C |
| ATOM | 2289 | CD2 | TRP | A | 291 | 20.191 | −4.581 | −11.187 | 1.00 | 37.81 | C |
| ATOM | 2290 | CE3 | TRP | A | 291 | 20.090 | −3.669 | −12.233 | 1.00 | 36.89 | C |
| ATOM | 2291 | CZ3 | TRP | A | 291 | 20.992 | −3.750 | −13.265 | 1.00 | 39.89 | C |
| ATOM | 2292 | CH2 | TRP | A | 291 | 22.002 | −4.714 | −13.277 | 1.00 | 41.26 | C |
| ATOM | 2293 | CZ2 | TRP | A | 291 | 22.143 | −5.619 | −12.252 | 1.00 | 40.73 | C |
| ATOM | 2294 | C | TRP | A | 291 | 16.678 | −5.318 | −10.995 | 1.00 | 46.55 | C |
| ATOM | 2295 | O | TRP | A | 291 | 16.215 | −6.124 | −10.200 | 1.00 | 43.24 | O |
| ATOM | 2296 | N | ASN | A | 292 | 16.854 | −5.518 | −12.306 | 1.00 | 52.83 | N |
| ATOM | 2297 | CA | ASN | A | 292 | 16.450 | −6.708 | −13.114 | 1.00 | 53.69 | C |
| ATOM | 2298 | CB | ASN | A | 292 | 16.183 | −6.361 | −14.584 | 1.00 | 54.03 | C |
| ATOM | 2299 | CG | ASN | A | 292 | 14.789 | −5.862 | −14.826 | 1.00 | 60.05 | C |
| ATOM | 2300 | OD1 | ASN | A | 292 | 13.853 | −6.311 | −14.181 | 1.00 | 72.16 | O |
| ATOM | 2301 | ND2 | ASN | A | 292 | 14.644 | −4.956 | −15.772 | 1.00 | 68.92 | N |
| ATOM | 2302 | C | ASN | A | 292 | 17.609 | −7.679 | −13.314 | 1.00 | 51.00 | C |
| ATOM | 2303 | O | ASN | A | 292 | 17.327 | −8.828 | −13.638 | 1.00 | 46.02 | O |
| ATOM | 2304 | N | GLY | A | 293 | 18.852 | −7.176 | −13.269 | 1.00 | 50.48 | N |
| ATOM | 2305 | CA | GLY | A | 293 | 20.036 | −7.841 | −13.843 | 1.00 | 45.90 | C |
| ATOM | 2306 | C | GLY | A | 293 | 20.655 | −8.845 | −12.883 | 1.00 | 43.27 | C |
| ATOM | 2307 | O | GLY | A | 293 | 20.067 | −9.239 | −11.877 | 1.00 | 42.71 | O |
| ATOM | 2308 | N | PRO | A | 294 | 21.890 | −9.281 | −13.184 | 1.00 | 48.42 | N |
| ATOM | 2309 | CA | PRO | A | 294 | 22.646 | −10.158 | −12.297 | 1.00 | 46.10 | C |
| ATOM | 2310 | CB | PRO | A | 294 | 24.063 | −10.057 | −12.861 | 1.00 | 48.66 | C |
| ATOM | 2311 | CG | PRO | A | 294 | 23.835 | −9.857 | −14.341 | 1.00 | 50.86 | C |
| ATOM | 2312 | CD | PRO | A | 294 | 22.638 | −8.934 | −14.401 | 1.00 | 52.15 | C |
| ATOM | 2313 | C | PRO | A | 294 | 22.651 | −9.676 | −10.847 | 1.00 | 44.37 | C |
| ATOM | 2314 | O | PRO | A | 294 | 22.970 | −8.526 | −10.631 | 1.00 | 49.09 | O |
| ATOM | 2315 | N | TRP | A | 295 | 22.311 | −10.592 | −9.943 | 1.00 | 40.98 | N |
| ATOM | 2316 | CA | TRP | A | 295 | 22.392 | −10.508 | −8.466 | 1.00 | 40.53 | C |
| ATOM | 2317 | CB | TRP | A | 295 | 23.691 | −9.825 | −8.036 | 1.00 | 43.17 | C |
| ATOM | 2318 | CG | TRP | A | 295 | 24.837 | −10.765 | −8.215 | 1.00 | 42.02 | C |
| ATOM | 2319 | CD1 | TRP | A | 295 | 25.737 | −10.783 | −9.237 | 1.00 | 44.52 | C |
| ATOM | 2320 | NE1 | TRP | A | 295 | 26.601 | −11.830 | −9.087 | 1.00 | 46.43 | N |
| ATOM | 2321 | CE2 | TRP | A | 295 | 26.241 | −12.547 | −7.979 | 1.00 | 45.46 | C |
| ATOM | 2322 | CD2 | TRP | A | 295 | 25.122 | −11.909 | −7.406 | 1.00 | 43.22 | C |
| ATOM | 2323 | CE3 | TRP | A | 295 | 24.553 | −12.444 | −6.249 | 1.00 | 48.14 | C |
| ATOM | 2324 | CZ3 | TRP | A | 295 | 25.106 | −13.580 | −5.708 | 1.00 | 50.67 | C |
| ATOM | 2325 | CH2 | TRP | A | 295 | 26.227 | −14.186 | −6.281 | 1.00 | 51.63 | C |
| ATOM | 2326 | CZ2 | TRP | A | 295 | 26.812 | −13.685 | −7.419 | 1.00 | 48.99 | C |
| ATOM | 2327 | C | TRP | A | 295 | 21.122 | −9.894 | −7.883 | 1.00 | 39.47 | C |
| ATOM | 2328 | O | TRP | A | 295 | 21.013 | −9.816 | −6.656 | 1.00 | 42.13 | O |
| ATOM | 2329 | N | SER | A | 296 | 20.137 | −9.610 | −8.722 | 1.00 | 39.15 | N |
| ATOM | 2330 | CA | SER | A | 296 | 18.749 | −9.395 | −8.254 | 1.00 | 44.43 | C |
| ATOM | 2331 | CB | SER | A | 296 | 17.875 | −8.873 | −9.349 | 1.00 | 45.33 | C |
| ATOM | 2332 | OG | SER | A | 296 | 18.024 | −9.684 | −10.490 | 1.00 | 43.40 | O |
| ATOM | 2333 | C | SER | A | 296 | 18.226 | −10.724 | −7.723 | 1.00 | 40.87 | C |
| ATOM | 2334 | O | SER | A | 296 | 18.670 | −11.769 | −8.230 | 1.00 | 37.99 | O |
| ATOM | 2335 | N | ASP | A | 297 | 17.309 | −10.685 | −6.760 | 1.00 | 41.61 | N |
| ATOM | 2336 | CA | ASP | A | 297 | 16.787 | −11.911 | −6.106 | 1.00 | 42.75 | C |
| ATOM | 2337 | CB | ASP | A | 297 | 15.960 | −11.604 | −4.862 | 1.00 | 44.08 | C |
| ATOM | 2338 | CG | ASP | A | 297 | 14.747 | −10.745 | −5.126 | 1.00 | 43.64 | C |
| ATOM | 2339 | OD1 | ASP | A | 297 | 14.502 | −10.425 | −6.291 | 1.00 | 47.37 | O |
| ATOM | 2340 | OD2 | ASP | A | 297 | 14.077 | −10.400 | −4.154 | 1.00 | 46.95 | O |
| ATOM | 2341 | C | ASP | A | 297 | 16.018 | −12.770 | −7.108 | 1.00 | 41.84 | C |
| ATOM | 2342 | O | ASP | A | 297 | 15.756 | −13.926 | −6.760 | 1.00 | 45.07 | O |
| ATOM | 2343 | N | THR | A | 298 | 15.718 | −12.253 | −8.301 | 1.00 | 41.62 | N |
| ATOM | 2344 | CA | THR | A | 298 | 14.995 | −12.971 | −9.384 | 1.00 | 43.94 | C |
| ATOM | 2345 | CB | THR | A | 298 | 13.957 | −12.038 | −10.015 | 1.00 | 49.37 | C |
| ATOM | 2346 | OG1 | THR | A | 298 | 14.763 | −10.997 | −10.571 | 1.00 | 50.65 | O |
| ATOM | 2347 | CG2 | THR | A | 298 | 12.949 | −11.474 | −9.034 | 1.00 | 46.13 | C |
| ATOM | 2348 | C | THR | A | 298 | 15.978 | −13.442 | −10.464 | 1.00 | 43.38 | C |
| ATOM | 2349 | O | THR | A | 298 | 15.512 | −13.859 | −11.521 | 1.00 | 40.86 | O |
| ATOM | 2350 | N | SER | A | 299 | 17.289 | −13.362 | −10.229 | 1.00 | 42.01 | N |
| ATOM | 2351 | CA | SER | A | 299 | 18.329 | −13.699 | −11.240 | 1.00 | 43.49 | C |
| ATOM | 2352 | CB | SER | A | 299 | 19.482 | −12.740 | −11.168 | 1.00 | 42.55 | C |
| ATOM | 2353 | OG | SER | A | 299 | 20.076 | −12.809 | −9.883 | 1.00 | 38.43 | O |
| ATOM | 2354 | C | SER | A | 299 | 18.819 | −15.124 | −11.001 | 1.00 | 46.98 | C |
| ATOM | 2355 | O | SER | A | 299 | 18.728 | −15.591 | −9.849 | 1.00 | 41.79 | O |
| ATOM | 2356 | N | GLU | A | 300 | 19.365 | −15.780 | −12.025 | 1.00 | 49.93 | N |
| ATOM | 2357 | CA | GLU | A | 300 | 19.910 | −17.148 | −11.853 | 1.00 | 56.46 | C |
| ATOM | 2358 | CB | GLU | A | 300 | 20.091 | −17.839 | −13.209 | 1.00 | 66.70 | C |
| ATOM | 2359 | CG | GLU | A | 300 | 21.377 | −17.506 | −13.945 | 1.00 | 73.59 | C |
| ATOM | 2360 | CD | GLU | A | 300 | 21.598 | −18.325 | −15.212 | 1.00 | 80.38 | C |
| ATOM | 2361 | OE1 | GLU | A | 300 | 20.883 | −19.349 | −15.421 | 1.00 | 73.39 | O |
| ATOM | 2362 | OE2 | GLU | A | 300 | 22.492 | −17.941 | −15.992 | 1.00 | 88.82 | O |
| ATOM | 2363 | C | GLU | A | 300 | 21.161 | −17.063 | −10.962 | 1.00 | 54.96 | C |
| ATOM | 2364 | O | GLU | A | 300 | 21.361 | −17.983 | −10.143 | 1.00 | 48.93 | O |
| ATOM | 2365 | N | GLU | A | 301 | 21.939 | −15.978 | −11.052 | 1.00 | 53.12 | N |
| ATOM | 2366 | CA | GLU | A | 301 | 23.129 | −15.769 | −10.180 | 1.00 | 50.67 | C |
| ATOM | 2367 | CB | GLU | A | 301 | 23.759 | −14.385 | −10.370 | 1.00 | 52.33 | C |
| ATOM | 2368 | CG | GLU | A | 301 | 24.511 | −14.216 | −11.684 | 1.00 | 48.31 | C |
| ATOM | 2369 | CD | GLU | A | 301 | 23.622 | −13.886 | −12.869 | 1.00 | 49.63 | C |
| ATOM | 2370 | OE1 | GLU | A | 301 | 22.393 | −13.848 | −12.690 | 1.00 | 52.01 | O |
| ATOM | 2371 | OE2 | GLU | A | 301 | 24.154 | −13.686 | −13.970 | 1.00 | 52.56 | O |
| ATOM | 2372 | C | GLU | A | 301 | 22.696 | −15.938 | −8.721 | 1.00 | 50.92 | C |
| ATOM | 2373 | O | GLU | A | 301 | 23.293 | −16.729 | −7.996 | 1.00 | 56.74 | O |
| ATOM | 2374 | N | TRP | A | 302 | 21.679 | −15.204 | −8.298 | 1.00 | 48.72 | N |
| ATOM | 2375 | CA | TRP | A | 302 | 21.216 | −15.206 | −6.890 | 1.00 | 43.04 | C |
| ATOM | 2376 | CB | TRP | A | 302 | 20.176 | −14.096 | −6.714 | 1.00 | 43.14 | C |
| ATOM | 2377 | CG | TRP | A | 302 | 19.569 | −14.016 | −5.353 | 1.00 | 38.44 | C |
| ATOM | 2378 | CD1 | TRP | A | 302 | 18.548 | −14.770 | −4.862 | 1.00 | 35.96 | C |
| ATOM | 2379 | NE1 | TRP | A | 302 | 18.307 | −14.433 | −3.558 | 1.00 | 40.15 | N |
| ATOM | 2380 | CE2 | TRP | A | 302 | 19.140 | −13.406 | −3.200 | 1.00 | 36.89 | C |
| ATOM | 2381 | CD2 | TRP | A | 302 | 19.964 | −13.128 | −4.303 | 1.00 | 33.05 | C |
| ATOM | 2382 | CE3 | TRP | A | 302 | 20.947 | −12.148 | −4.181 | 1.00 | 35.40 | C |
| ATOM | 2383 | CZ3 | TRP | A | 302 | 21.062 | −11.469 | −2.989 | 1.00 | 38.66 | C |
| ATOM | 2384 | CH2 | TRP | A | 302 | 20.224 | −11.742 | −1.908 | 1.00 | 36.94 | C |
| ATOM | 2385 | CZ2 | TRP | A | 302 | 19.247 | −12.709 | −1.995 | 1.00 | 38.90 | C |
| ATOM | 2386 | C | TRP | A | 302 | 20.658 | −16.592 | −6.554 | 1.00 | 46.26 | C |
| ATOM | 2387 | O | TRP | A | 302 | 20.917 | −17.095 | −5.447 | 1.00 | 47.94 | O |
| ATOM | 2388 | N | GLN | A | 303 | 19.892 | −17.183 | −7.472 | 1.00 | 53.24 | N |
| ATOM | 2389 | CA | GLN | A | 303 | 19.207 | −18.480 | −7.234 | 1.00 | 53.22 | C |
| ATOM | 2390 | CB | GLN | A | 303 | 18.160 | −18.757 | −8.301 | 1.00 | 55.28 | C |
| ATOM | 2391 | CG | GLN | A | 303 | 16.897 | −17.950 | −8.099 | 1.00 | 56.42 | C |
| ATOM | 2392 | CD | GLN | A | 303 | 16.011 | −18.032 | −9.313 | 1.00 | 59.27 | C |
| ATOM | 2393 | OE1 | GLN | A | 303 | 16.216 | −18.854 | −10.204 | 1.00 | 56.75 | O |
| ATOM | 2394 | NE2 | GLN | A | 303 | 15.015 | −17.167 | −9.351 | 1.00 | 66.53 | N |
| ATOM | 2395 | C | GLN | A | 303 | 20.240 | −19.599 | −7.210 | 1.00 | 54.63 | C |
| ATOM | 2396 | O | GLN | A | 303 | 19.971 | −20.613 | −6.544 | 1.00 | 53.19 | O |
| ATOM | 2397 | N | LYS | A | 304 | 21.387 | −19.403 | −7.869 | 1.00 | 59.70 | N |
| ATOM | 2398 | CA | LYS | A | 304 | 22.460 | −20.437 | −7.957 | 1.00 | 66.62 | C |
| ATOM | 2399 | CB | LYS | A | 304 | 23.517 | −20.098 | −9.013 | 1.00 | 72.23 | C |
| ATOM | 2400 | CG | LYS | A | 304 | 24.012 | −21.297 | −9.819 | 1.00 | 87.27 | C |
| ATOM | 2401 | CD | LYS | A | 304 | 22.935 | −21.944 | −10.709 | 1.00 | 93.99 | C |
| ATOM | 2402 | CE | LYS | A | 304 | 23.431 | −23.087 | −11.575 | 1.00 | 96.36 | C |
| ATOM | 2403 | NZ | LYS | A | 304 | 24.332 | −24.000 | −10.833 | 1.00 | 99.39 | N |
| ATOM | 2404 | C | LYS | A | 304 | 23.126 | −20.629 | −6.589 | 1.00 | 65.96 | C |
| ATOM | 2405 | O | LYS | A | 304 | 23.752 | −21.689 | −6.405 | 1.00 | 68.54 | O |
| ATOM | 2406 | N | VAL | A | 305 | 22.960 | −19.666 | −5.670 | 1.00 | 56.60 | N |
| ATOM | 2407 | CA | VAL | A | 305 | 23.637 | −19.617 | −4.339 | 1.00 | 54.40 | C |
| ATOM | 2408 | CB | VAL | A | 305 | 24.358 | −18.279 | −4.103 | 1.00 | 56.30 | C |
| ATOM | 2409 | CG1 | VAL | A | 305 | 25.232 | −18.336 | −2.864 | 1.00 | 57.90 | C |
| ATOM | 2410 | CG2 | VAL | A | 305 | 25.177 | −17.852 | −5.308 | 1.00 | 9.28 | C |
| ATOM | 2411 | C | VAL | A | 305 | 22.597 | −19.837 | −3.249 | 1.00 | 49.93 | C |
| ATOM | 2412 | O | VAL | A | 305 | 21.534 | −19.176 | −3.289 | 1.00 | 49.58 | O |
| ATOM | 2413 | N | SER | A | 306 | 22.920 | −20.696 | −2.288 | 1.00 | 48.41 | N |
| ATOM | 2414 | CA | SER | A | 306 | 22.026 | −21.033 | −1.155 | 1.00 | 51.04 | C |
| ATOM | 2415 | CB | SER | A | 306 | 22.484 | −22.284 | −0.454 | 1.00 | 55.57 | C |
| ATOM | 2416 | OG | SER | A | 306 | 23.890 | −22.256 | −0.228 | 1.00 | 60.36 | O |
| ATOM | 2417 | C | SER | A | 306 | 21.965 | −19.840 | −0.198 | 1.00 | 48.10 | C |
| ATOM | 2418 | O | SER | A | 306 | 22.959 | −19.086 | −0.092 | 1.00 | 52.06 | O |
| ATOM | 2419 | N | LYS | A | 307 | 20.836 | −19.689 | 0.478 | 1.00 | 42.31 | N |
| ATOM | 2420 | CA | LYS | A | 307 | 20.649 | −18.692 | 1.556 | 1.00 | 44.05 | C |
| ATOM | 2421 | CB | LYS | A | 307 | 19.276 | −18.884 | 2.197 | 1.00 | 39.68 | C |
| ATOM | 2422 | CG | LYS | A | 307 | 19.067 | −18.127 | 3.485 | 1.00 | 43.23 | C |
| ATOM | 2423 | CD | LYS | A | 307 | 17.656 | −17.631 | 3.684 | 1.00 | 44.38 | C |
| ATOM | 2424 | CE | LYS | A | 307 | 17.388 | −17.419 | 5.157 | 1.00 | 49.31 | C |
| ATOM | 2425 | NZ | LYS | A | 307 | 16.502 | −16.258 | 5.385 | 1.00 | 55.57 | N |
| ATOM | 2426 | C | LYS | A | 307 | 21.832 | −18.815 | 2.518 | 1.00 | 47.45 | C |
| ATOM | 2427 | O | LYS | A | 307 | 22.435 | −17.787 | 2.842 | 1.00 | 52.73 | O |
| ATOM | 2428 | N | SER | A | 308 | 22.194 | −20.042 | 2.883 | 1.00 | 53.42 | N |
| ATOM | 2429 | CA | SER | A | 308 | 23.375 | −20.395 | 3.726 | 1.00 | 53.67 | C |
| ATOM | 2430 | CB | SER | A | 308 | 23.456 | −21.903 | 3.877 | 1.00 | 51.52 | C |
| ATOM | 2431 | OG | SER | A | 308 | 24.510 | −22.269 | 4.747 | 1.00 | 51.48 | O |
| ATOM | 2432 | C | SER | A | 308 | 24.686 | −19.783 | 3.179 | 1.00 | 49.88 | C |
| ATOM | 2433 | O | SER | A | 308 | 25.371 | −19.081 | 3.954 | 1.00 | 54.60 | O |
| ATOM | 2434 | N | GLU | A | 309 | 25.028 | −20.019 | 1.908 | 1.00 | 44.10 | N |
| ATOM | 2435 | CA | GLU | A | 309 | 26.265 | −19.498 | 1.253 | 1.00 | 49.20 | C |
| ATOM | 2436 | CB | GLU | A | 309 | 26.406 | −20.027 | −0.180 | 1.00 | 54.10 | C |
| ATOM | 2437 | CG | GLU | A | 309 | 27.647 | −20.876 | −0.429 | 1.00 | 57.58 | C |
| ATOM | 2438 | CD | GLU | A | 309 | 28.960 | −20.120 | −0.301 | 1.00 | 62.00 | C |
| ATOM | 2439 | OE1 | GLU | A | 309 | 29.653 | −19.953 | −1.329 | 1.00 | 67.67 | O |
| ATOM | 2440 | OE2 | GLU | A | 309 | 29.293 | −19.687 | 0.826 | 1.00 | 71.51 | O |
| ATOM | 2441 | C | GLU | A | 309 | 26.274 | −17.958 | 1.211 | 1.00 | 53.66 | C |
| ATOM | 2442 | O | GLU | A | 309 | 27.354 | −17.371 | 1.375 | 1.00 | 62.36 | O |
| ATOM | 2443 | N | ARG | A | 310 | 25.133 | −17.311 | 0.957 | 1.00 | 53.12 | N |
| ATOM | 2444 | CA | ARG | A | 310 | 25.033 | −15.830 | 0.890 | 1.00 | 51.74 | C |
| ATOM | 2445 | CB | ARG | A | 310 | 23.695 | −15.376 | 0.306 | 1.00 | 54.06 | C |
| ATOM | 2446 | CG | ARG | A | 310 | 23.526 | −15.726 | −1.161 | 1.00 | 53.72 | C |
| ATOM | 2447 | CD | ARG | A | 310 | 22.332 | −15.041 | −1.758 | 1.00 | 52.83 | C |
| ATOM | 2448 | NE | ARG | A | 310 | 21.215 | −14.948 | −0.835 | 1.00 | 50.33 | N |
| ATOM | 2449 | CZ | ARG | A | 310 | 20.135 | −15.719 | −0.847 | 1.00 | 49.99 | C |
| ATOM | 2450 | NH1 | ARG | A | 310 | 20.018 | −16.701 | −1.728 | 1.00 | 53.73 | N |
| ATOM | 2451 | NH2 | ARG | A | 310 | 19.173 | −15.498 | 0.038 | 1.00 | 49.69 | N |
| ATOM | 2452 | C | ARG | A | 310 | 25.204 | −15.233 | 2.286 | 1.00 | 50.34 | C |
| ATOM | 2453 | O | ARG | A | 310 | 25.912 | −14.242 | 2.382 | 1.00 | 50.02 | O |
| ATOM | 2454 | N | GLU | A | 311 | 24.583 | −15.804 | 3.320 | 1.00 | 48.55 | N |
| ATOM | 2455 | CA | GLU | A | 311 | 24.724 | −15.282 | 4.703 | 1.00 | 51.61 | C |
| ATOM | 2456 | CB | GLU | A | 311 | 23.836 | −16.056 | 5.674 | 1.00 | 53.83 | C |
| ATOM | 2457 | CG | GLU | A | 311 | 22.354 | −15.895 | 5.380 | 1.00 | 61.46 | C |
| ATOM | 2458 | CD | GLU | A | 311 | 21.418 | −16.698 | 6.266 | 1.00 | 66.12 | C |
| ATOM | 2459 | OE1 | GLU | A | 311 | 20.237 | −16.314 | 6.359 | 1.00 | 69.56 | O |
| ATOM | 2460 | OE2 | GLU | A | 311 | 21.872 | −17.694 | 6.868 | 1.00 | 75.65 | O |
| ATOM | 2461 | C | GLU | A | 311 | 26.215 | −15.303 | 5.093 | 1.00 | 56.38 | C |
| ATOM | 2462 | O | GLU | A | 311 | 26.686 | −14.314 | 5.699 | 1.00 | 55.09 | O |
| ATOM | 2463 | N | LYS | A | 312 | 26.959 | −16.343 | 4.713 | 1.00 | 58.06 | N |
| ATOM | 2464 | CA | LYS | A | 312 | 28.399 | −16.437 | 5.065 | 1.00 | 64.81 | C |
| ATOM | 2465 | CB | LYS | A | 312 | 28.947 | −17.857 | 4.895 | 1.00 | 68.95 | C |
| ATOM | 2466 | CG | LYS | A | 312 | 30.462 | −17.986 | 5.031 | 1.00 | 73.60 | C |
| ATOM | 2467 | CD | LYS | A | 312 | 31.180 | −17.983 | 3.690 | 1.00 | 80.54 | C |
| ATOM | 2468 | CE | LYS | A | 312 | 32.670 | −17.727 | 3.799 | 1.00 | 83.81 | C |
| ATOM | 2469 | NZ | LYS | A | 312 | 33.411 | −18.940 | 4.212 | 1.00 | 81.90 | N |
| ATOM | 2470 | C | LYS | A | 312 | 29.173 | −15.417 | 4.225 | 1.00 | 61.12 | C |
| ATOM | 2471 | O | LYS | A | 312 | 30.121 | −14.832 | 4.757 | 1.00 | 69.20 | O |
| ATOM | 2472 | N | MET | A | 313 | 28.782 | −15.214 | 2.971 | 1.00 | 53.45 | N |
| ATOM | 2473 | CA | MET | A | 313 | 29.433 | −14.247 | 2.052 | 1.00 | 53.61 | C |
| ATOM | 2474 | CB | MET | A | 313 | 29.006 | −14.502 | 0.605 | 1.00 | 57.57 | C |
| ATOM | 2475 | CG | MET | A | 313 | 29.681 | −15.696 | −0.021 | 1.00 | 58.81 | C |
| ATOM | 2476 | SD | MET | A | 313 | 29.508 | −15.632 | −1.823 | 1.00 | 74.12 | S |
| ATOM | 2477 | CE | MET | A | 313 | 27.744 | −15.913 | −1.976 | 1.00 | 64.60 | C |
| ATOM | 2478 | C | MET | A | 313 | 29.054 | −12.809 | 2.434 | 1.00 | 48.75 | C |
| ATOM | 2479 | O | MET | A | 313 | 29.696 | −11.873 | 1.914 | 1.00 | 45.24 | O |
| ATOM | 2480 | N | GLY | A | 314 | 28.051 | −12.646 | 3.301 | 1.00 | 48.42 | N |
| ATOM | 2481 | CA | GLY | A | 314 | 27.442 | −11.346 | 3.654 | 1.00 | 51.27 | C |
| ATOM | 2482 | C | GLY | A | 314 | 26.646 | −10.722 | 2.508 | 1.00 | 48.73 | C |
| ATOM | 2483 | O | GLY | A | 314 | 26.478 | −9.478 | 2.519 | 1.00 | 48.83 | O |
| ATOM | 2484 | N | VAL | A | 315 | 26.198 | −11.517 | 1.529 | 1.00 | 40.79 | N |
| ATOM | 2485 | CA | VAL | A | 315 | 25.277 | −11.032 | 0.465 | 1.00 | 39.12 | C |
| ATOM | 2486 | CB | VAL | A | 315 | 25.552 | −11.727 | −0.876 | 1.00 | 39.43 | C |
| ATOM | 2487 | CG1 | VAL | A | 315 | 24.583 | −11.277 | −1.951 | 1.00 | 36.86 | C |
| ATOM | 2488 | CG2 | VAL | A | 315 | 26.981 | −11.459 | −1.333 | 1.00 | 38.66 | C |
| ATOM | 2489 | C | VAL | A | 315 | 23.841 | −11.199 | 0.968 | 1.00 | 40.42 | C |
| ATOM | 2490 | O | VAL | A | 315 | 23.147 | −12.128 | 0.512 | 1.00 | 43.61 | O |
| ATOM | 2491 | N | THR | A | 316 | 23.423 | −10.333 | 1.897 | 1.00 | 38.54 | N |
| ATOM | 2492 | CA | THR | A | 316 | 22.040 | −10.282 | 2.427 | 1.00 | 35.98 | C |
| ATOM | 2493 | CB | THR | A | 316 | 22.009 | −10.733 | 3.886 | 1.00 | 37.07 | C |
| ATOM | 2494 | OG1 | THR | A | 316 | 22.643 | −9.715 | 4.636 | 1.00 | 37.32 | O |
| ATOM | 2495 | CG2 | THR | A | 316 | 22.721 | −12.040 | 4.136 | 1.00 | 41.91 | C |
| ATOM | 2496 | C | THR | A | 316 | 21.455 | −8.863 | 2.316 | 1.00 | 36.90 | C |
| ATOM | 2497 | O | THR | A | 316 | 22.205 | −7.911 | 1.990 | 1.00 | 35.61 | O |
| ATOM | 2498 | N | VAL | A | 317 | 20.161 | −8.720 | 2.610 | 1.00 | 34.64 | N |
| ATOM | 2499 | CA | VAL | A | 317 | 19.440 | −7.426 | 2.523 | 1.00 | 38.10 | C |
| ATOM | 2500 | CB | VAL | A | 317 | 18.520 | −7.401 | 1.299 | 1.00 | 39.17 | C |
| ATOM | 2501 | CG1 | VAL | A | 317 | 19.318 | −7.364 | 0.007 | 1.00 | 38.01 | C |
| ATOM | 2502 | CG2 | VAL | A | 317 | 17.537 | −8.572 | 1.316 | 1.00 | 38.66 | C |
| ATOM | 2503 | C | VAL | A | 317 | 18.640 | −7.246 | 3.802 | 1.00 | 40.65 | C |
| ATOM | 2504 | O | VAL | A | 317 | 18.118 | −8.245 | 4.261 | 1.00 | 39.84 | O |
| ATOM | 2505 | N | GLN | A | 318 | 18.532 | −6.021 | 4.323 | 1.00 | 46.38 | N |
| ATOM | 2506 | CA | GLN | A | 318 | 17.635 | −5.715 | 5.472 | 1.00 | 55.66 | C |
| ATOM | 2507 | CB | GLN | A | 318 | 17.957 | −4.359 | 6.119 | 1.00 | 63.55 | C |
| ATOM | 2508 | CG | GLN | A | 318 | 16.735 | −3.688 | 6.760 | 1.00 | 74.27 | C |
| ATOM | 2509 | CD | GLN | A | 318 | 16.988 | −2.410 | 7.539 | 1.00 | 77.61 | C |
| ATOM | 2510 | OE1 | GLN | A | 318 | 17.093 | −1.307 | 6.988 | 1.00 | 62.35 | O |
| ATOM | 2511 | NE2 | GLN | A | 318 | 17.016 | −2.540 | 8.859 | 1.00 | 80.34 | N |
| ATOM | 2512 | C | GLN | A | 318 | 16.174 | −5.810 | 4.988 | 1.00 | 57.54 | C |
| ATOM | 2513 | O | GLN | A | 318 | 15.323 | −6.195 | 5.813 | 1.00 | 60.72 | O |
| ATOM | 2514 | N | ASP | A | 319 | 15.884 | −5.485 | 3.719 | 1.00 | 49.24 | N |
| ATOM | 2515 | CA | ASP | A | 319 | 14.495 | −5.492 | 3.198 | 1.00 | 49.55 | C |
| ATOM | 2516 | CB | ASP | A | 319 | 13.675 | −4.362 | 3.816 | 1.00 | 57.30 | C |
| ATOM | 2517 | CG | ASP | A | 319 | 14.386 | −3.026 | 3.732 | 1.00 | 61.26 | C |
| ATOM | 2518 | OD1 | ASP | A | 319 | 14.800 | −2.638 | 2.610 | 1.00 | 71.01 | O |
| ATOM | 2519 | OD2 | ASP | A | 319 | 14.558 | −2.409 | 4.793 | 1.00 | 62.33 | O |
| ATOM | 2520 | C | ASP | A | 319 | 14.460 | −5.329 | 1.679 | 1.00 | 47.28 | C |
| ATOM | 2521 | O | ASP | A | 319 | 15.507 | −5.262 | 1.072 | 1.00 | 49.78 | O |
| ATOM | 2522 | N | ASP | A | 320 | 13.226 | −5.310 | 1.175 | 1.00 | 49.46 | N |
| ATOM | 2523 | CA | ASP | A | 320 | 12.686 | −5.176 | −0.198 | 1.00 | 46.48 | C |
| ATOM | 2524 | CB | ASP | A | 320 | 11.194 | −4.813 | −0.091 | 1.00 | 57.09 | C |
| ATOM | 2525 | CG | ASP | A | 320 | 10.229 | −5.727 | −0.835 | 1.00 | 70.17 | C |
| ATOM | 2526 | OD1 | ASP | A | 320 | 10.693 | −6.580 | −1.620 | 1.00 | 73.02 | O |
| ATOM | 2527 | OD2 | ASP | A | 320 | 9.005 | −5.577 | −0.624 | 1.00 | 79.93 | O |
| ATOM | 2528 | C | ASP | A | 320 | 13.336 | −4.047 | −1.001 | 1.00 | 40.62 | C |
| ATOM | 2529 | O | ASP | A | 320 | 13.352 | −4.151 | −2.226 | 1.00 | 34.17 | O |
| ATOM | 2530 | N | GLY | A | 321 | 13.724 | −2.945 | −0.363 | 1.00 | 38.74 | N |
| ATOM | 2531 | CA | GLY | A | 321 | 14.310 | −1.789 | −1.069 | 1.00 | 34.98 | C |
| ATOM | 2532 | C | GLY | A | 321 | 15.775 | −1.979 | −1.420 | 1.00 | 33.81 | C |
| ATOM | 2533 | O | GLY | A | 321 | 16.293 | −1.174 | −2.212 | 1.00 | 32.96 | O |
| ATOM | 2534 | N | GLU | A | 322 | 16.452 | −2.983 | −0.870 | 1.00 | 34.08 | N |
| ATOM | 2535 | CA | GLU | A | 322 | 17.910 | −3.158 | −1.139 | 1.00 | 40.61 | C |
| ATOM | 2536 | CB | GLU | A | 322 | 18.749 | −3.541 | 0.078 | 1.00 | 39.78 | C |
| ATOM | 2537 | CG | GLU | A | 322 | 18.542 | −2.616 | 1.251 | 1.00 | 43.80 | C |
| ATOM | 2538 | CD | GLU | A | 322 | 19.558 | −2.815 | 2.358 | 1.00 | 45.19 | C |
| ATOM | 2539 | OE1 | GLU | A | 322 | 19.599 | −3.933 | 2.952 | 1.00 | 46.07 | O |
| ATOM | 2540 | OE2 | GLU | A | 322 | 20.312 | −1.854 | 2.617 | 1.00 | 40.20 | O |
| ATOM | 2541 | C | GLU | A | 322 | 18.109 | −4.215 | −2.212 | 1.00 | 39.13 | C |
| ATOM | 2542 | O | GLU | A | 322 | 17.245 | −5.062 | −2.400 | 1.00 | 38.52 | O |
| ATOM | 2543 | N | PHE | A | 323 | 19.256 | −4.152 | −2.860 | 1.00 | 38.97 | N |
| ATOM | 2544 | CA | PHE | A | 323 | 19.585 | −5.015 | −4.003 | 1.00 | 38.75 | C |
| ATOM | 2545 | CB | PHE | A | 323 | 18.777 | −4.585 | −5.212 | 1.00 | 35.72 | C |
| ATOM | 2546 | CG | PHE | A | 323 | 19.005 | −3.167 | −5.649 | 1.00 | 37.61 | C |
| ATOM | 2547 | CD1 | PHE | A | 323 | 18.362 | −2.114 | −5.017 | 1.00 | 35.72 | C |
| ATOM | 2548 | CE1 | PHE | A | 323 | 18.541 | −0.812 | −5.457 | 1.00 | 34.22 | C |
| ATOM | 2549 | CZ | PHE | A | 323 | 19.347 | −0.547 | −6.535 | 1.00 | 34.83 | C |
| ATOM | 2550 | CD2 | PHE | A | 323 | 19.799 | −2.892 | −6.750 | 1.00 | 39.27 | C |
| ATOM | 2551 | CE2 | PHE | A | 323 | 19.996 | −1.581 | −7.168 | 1.00 | 40.10 | C |
| ATOM | 2552 | C | PHE | A | 323 | 21.091 | −4.987 | −4.248 | 1.00 | 38.47 | C |
| ATOM | 2553 | O | PHE | A | 323 | 21.767 | −3.998 | −3.895 | 1.00 | 39.17 | O |
| ATOM | 2554 | N | TRP | A | 324 | 21.581 | −6.091 | −4.800 | 1.00 | 33.12 | N |
| ATOM | 2555 | CA | TRP | A | 324 | 23.018 | −6.352 | −4.985 | 1.00 | 36.19 | C |
| ATOM | 2556 | CB | TRP | A | 324 | 23.373 | −7.726 | −4.414 | 1.00 | 35.11 | C |
| ATOM | 2557 | CG | TRP | A | 324 | 23.468 | −7.734 | −2.924 | 1.00 | 31.18 | C |
| ATOM | 2558 | CD1 | TRP | A | 324 | 22.492 | −8.039 | −2.023 | 1.00 | 32.46 | C |
| ATOM | 2559 | NE1 | TRP | A | 324 | 22.959 | −7.929 | −0.737 | 1.00 | 30.44 | N |
| ATOM | 2560 | CE2 | TRP | A | 324 | 24.285 | −7.578 | −0.788 | 1.00 | 34.07 | C |
| ATOM | 2561 | CD2 | TRP | A | 324 | 24.632 | −7.423 | −2.147 | 1.00 | 35.21 | C |
| ATOM | 2562 | CE3 | TRP | A | 324 | 25.934 | −7.042 | −2.468 | 1.00 | 37.30 | C |
| ATOM | 2563 | CZ3 | TRP | A | 324 | 26.827 | −6.811 | −1.450 | 1.00 | 36.19 | C |
| ATOM | 2564 | CH2 | TRP | A | 324 | 26.459 | −6.940 | −0.118 | 1.00 | 32.45 | C |
| ATOM | 2565 | CZ2 | TRP | A | 324 | 25.196 | −7.338 | 0.238 | 1.00 | 35.15 | C |
| ATOM | 2566 | C | TRP | A | 324 | 23.347 | −6.212 | −6.467 | 1.00 | 37.59 | C |
| ATOM | 2567 | O | TRP | A | 324 | 22.530 | −6.650 | −7.317 | 1.00 | 36.71 | O |
| ATOM | 2568 | N | MET | A | 325 | 24.478 | −5.577 | −6.762 | 1.00 | 36.16 | N |
| ATOM | 2569 | CA | MET | A | 325 | 25.029 | −5.515 | −8.133 | 1.00 | 38.83 | C |
| ATOM | 2570 | CB | MET | A | 325 | 24.764 | −4.168 | −8.792 | 1.00 | 37.42 | C |
| ATOM | 2571 | CG | MET | A | 325 | 23.309 | −3.820 | −8.829 | 1.00 | 41.68 | C |
| ATOM | 2572 | SD | MET | A | 325 | 23.129 | −2.169 | −9.424 | 1.00 | 40.45 | S |
| ATOM | 2573 | CE | MET | A | 325 | 23.581 | −2.404 | −11.146 | 1.00 | 37.24 | C |
| ATOM | 2574 | C | MET | A | 325 | 26.532 | −5.730 | −8.028 | 1.00 | 44.62 | C |
| ATOM | 2575 | O | MET | A | 325 | 27.098 | −5.414 | −6.939 | 1.00 | 44.80 | O |
| ATOM | 2576 | N | THR | A | 326 | 27.122 | −6.282 | −9.088 | 1.00 | 42.23 | N |
| ATOM | 2577 | CA | THR | A | 326 | 28.588 | −6.305 | −9.266 | 1.00 | 45.66 | C |
| ATOM | 2578 | CB | THR | A | 326 | 29.031 | −7.252 | −10.393 | 1.00 | 46.32 | C |
| ATOM | 2579 | OG1 | THR | A | 326 | 28.472 | −6.826 | −11.633 | 1.00 | 48.91 | O |
| ATOM | 2580 | CG2 | THR | A | 326 | 28.627 | −8.689 | −10.140 | 1.00 | 49.88 | C |
| ATOM | 2581 | C | THR | A | 326 | 28.967 | −4.846 | −9.484 | 1.00 | 48.19 | C |
| ATOM | 2582 | O | THR | A | 326 | 28.128 | −4.101 | −10.053 | 1.00 | 43.34 | O |
| ATOM | 2583 | N | PHE | A | 327 | 30.160 | −4.450 | −9.044 | 1.00 | 49.51 | N |
| ATOM | 2584 | CA | PHE | A | 327 | 30.710 | −3.113 | −9.353 | 1.00 | 52.35 | C |
| ATOM | 2585 | CB | PHE | A | 327 | 32.046 | −2.865 | −8.655 | 1.00 | 60.22 | C |
| ATOM | 2586 | CG | PHE | A | 327 | 32.420 | −1.411 | −8.695 | 1.00 | 66.15 | C |
| ATOM | 2587 | CD1 | PHE | A | 327 | 31.666 | −0.485 | −7.994 | 1.00 | 70.79 | C |
| ATOM | 2588 | CE1 | PHE | A | 327 | 31.971 | 0.865 | −8.061 | 1.00 | 77.69 | C |
| ATOM | 2589 | CZ | PHE | A | 327 | 33.024 | 1.299 | −8.839 | 1.00 | 79.41 | C |
| ATOM | 2590 | CD2 | PHE | A | 327 | 33.447 | −0.958 | −9.508 | 1.00 | 69.84 | C |
| ATOM | 2591 | CE2 | PHE | A | 327 | 33.766 | 0.390 | −9.560 | 1.00 | 68.02 | C |
| ATOM | 2592 | C | PHE | A | 327 | 30.812 | −2.960 | −10.875 | 1.00 | 52.35 | C |
| ATOM | 2593 | O | PHE | A | 327 | 30.779 | −1.819 | −11.360 | 1.00 | 58.22 | O |
| ATOM | 2594 | N | GLU | A | 328 | 30.887 | −4.062 | −11.621 | 1.00 | 52.62 | N |
| ATOM | 2595 | CA | GLU | A | 328 | 30.953 | −4.003 | −13.103 | 1.00 | 59.90 | C |
| ATOM | 2596 | CB | GLU | A | 328 | 31.392 | −5.337 | −13.715 | 1.00 | 65.05 | C |
| ATOM | 2597 | CG | GLU | A | 328 | 31.553 | −5.278 | −15.231 | 1.00 | 71.76 | C |
| ATOM | 2598 | CD | GLU | A | 328 | 32.185 | −6.479 | −15.934 | 1.00 | 76.10 | C |
| ATOM | 2599 | OE1 | GLU | A | 328 | 32.527 | −7.483 | −15.261 | 1.00 | 74.18 | O |
| ATOM | 2600 | OE2 | GLU | A | 328 | 32.337 | −6.404 | −17.171 | 1.00 | 81.13 | O |
| ATOM | 2601 | C | GLU | A | 328 | 29.591 | −3.528 | −13.618 | 1.00 | 61.37 | C |
| ATOM | 2602 | O | GLU | A | 328 | 29.572 | −2.727 | −14.566 | 1.00 | 63.41 | O |
| ATOM | 2603 | N | ASP | A | 329 | 28.486 | −3.969 | −13.012 | 1.00 | 57.51 | N |
| ATOM | 2604 | CA | ASP | A | 329 | 27.130 | −3.540 | −13.453 | 1.00 | 54.87 | C |
| ATOM | 2605 | CB | ASP | A | 329 | 26.061 | −4.530 | −13.000 | 1.00 | 57.62 | C |
| ATOM | 2606 | CG | ASP | A | 329 | 25.991 | −5.727 | −13.927 | 1.00 | 57.72 | C |
| ATOM | 2607 | OD1 | ASP | A | 329 | 26.050 | −5.495 | −15.151 | 1.00 | 59.10 | O |
| ATOM | 2608 | OD2 | ASP | A | 329 | 25.888 | −6.868 | −13.419 | 1.00 | 52.48 | O |
| ATOM | 2609 | C | ASP | A | 329 | 26.859 | −2.110 | −12.981 | 1.00 | 50.81 | C |
| ATOM | 2610 | O | ASP | A | 329 | 26.255 | −1.348 | −13.755 | 1.00 | 50.35 | O |
| ATOM | 2611 | N | VAL | A | 330 | 27.310 | −1.750 | −11.779 | 1.00 | 48.78 | N |
| ATOM | 2612 | CA | VAL | A | 330 | 27.250 | −0.345 | −11.272 | 1.00 | 50.12 | C |
| ATOM | 2613 | CB | VAL | A | 330 | 27.978 | −0.195 | −9.926 | 1.00 | 48.53 | C |
| ATOM | 2614 | CG1 | VAL | A | 330 | 28.055 | 1.255 | −9.494 | 1.00 | 50.35 | C |
| ATOM | 2615 | CG2 | VAL | A | 330 | 27.336 | −1.033 | −8.832 | 1.00 | 47.25 | C |
| ATOM | 2616 | C | VAL | A | 330 | 27.843 | 0.598 | −12.336 | 1.00 | 53.10 | C |
| ATOM | 2617 | O | VAL | A | 330 | 27.154 | 1.554 | −12.753 | 1.00 | 54.12 | O |
| ATOM | 2618 | N | CYS | A | 331 | 29.063 | 0.333 | −12.804 | 1.00 | 53.67 | N |
| ATOM | 2619 | CA | CYS | A | 331 | 29.747 | 1.194 | −13.809 | 1.00 | 54.08 | C |
| ATOM | 2620 | CB | CYS | A | 331 | 31.223 | 0.852 | −13.913 | 1.00 | 53.26 | C |
| ATOM | 2621 | SG | CYS | A | 331 | 32.081 | 1.232 | −12.368 | 1.00 | 57.21 | S |
| ATOM | 2622 | C | CYS | A | 331 | 29.053 | 1.133 | −15.172 | 1.00 | 52.70 | C |
| ATOM | 2623 | O | CYS | A | 331 | 29.159 | 2.108 | −15.906 | 1.00 | 58.64 | O |
| ATOM | 2624 | N | ARG | A | 332 | 28.346 | 0.049 | −15.484 | 1.00 | 58.51 | N |
| ATOM | 2625 | CA | ARG | A | 332 | 27.657 | −0.128 | −16.790 | 1.00 | 61.98 | C |
| ATOM | 2626 | CB | ARG | A | 332 | 27.441 | −1.625 | −17.042 | 1.00 | 70.22 | C |
| ATOM | 2627 | CG | ARG | A | 332 | 26.665 | −1.975 | −18.308 | 1.00 | 78.36 | C |
| ATOM | 2628 | CD | ARG | A | 332 | 26.964 | −3.393 | −18.794 | 1.00 | 84.57 | C |
| ATOM | 2629 | NE | ARG | A | 332 | 28.342 | −3.511 | −19.284 | 1.00 | 95.56 | N |
| ATOM | 2630 | CZ | ARG | A | 332 | 29.322 | −4.270 | −18.767 | 1.00 | 93.72 | C |
| ATOM | 2631 | NH1 | ARG | A | 332 | 29.119 | −5.055 | −17.718 | 1.00 | 89.88 | N |
| ATOM | 2632 | NH2 | ARG | A | 332 | 30.521 | −4.239 | −19.327 | 1.00 | 87.62 | N |
| ATOM | 2633 | C | ARG | A | 332 | 26.363 | 0.711 | −16.829 | 1.00 | 56.83 | C |
| ATOM | 2634 | O | ARG | A | 332 | 26.076 | 1.269 | −17.894 | 1.00 | 60.83 | O |
| ATOM | 2635 | N | TYR | A | 333 | 25.605 | 0.832 | −15.732 | 1.00 | 56.16 | N |
| ATOM | 2636 | CA | TYR | A | 333 | 24.226 | 1.409 | −15.758 | 1.00 | 55.32 | C |
| ATOM | 2637 | CB | TYR | A | 333 | 23.214 | 0.395 | −15.213 | 1.00 | 57.67 | C |
| ATOM | 2638 | CG | TYR | A | 333 | 23.033 | −0.850 | −16.049 | 1.00 | 56.93 | C |
| ATOM | 2639 | CD1 | TYR | A | 333 | 22.173 | −0.877 | −17.140 | 1.00 | 61.06 | C |
| ATOM | 2640 | CE1 | TYR | A | 333 | 21.997 | −2.027 | −17.899 | 1.00 | 59.38 | C |
| ATOM | 2641 | CZ | TYR | A | 333 | 22.693 | −3.181 | −17.578 | 1.00 | 58.82 | C |
| ATOM | 2642 | OH | TYR | A | 333 | 22.543 | −4.326 | −18.312 | 1.00 | 66.07 | O |
| ATOM | 2643 | CE2 | TYR | A | 333 | 23.562 | −3.168 | −16.502 | 1.00 | 59.87 | C |
| ATOM | 2644 | CD2 | TYR | A | 333 | 23.725 | −2.011 | −15.752 | 1.00 | 59.03 | C |
| ATOM | 2645 | C | TYR | A | 333 | 24.141 | 2.758 | −15.024 | 1.00 | 49.99 | C |
| ATOM | 2646 | O | TYR | A | 333 | 23.204 | 3.515 | −15.295 | 1.00 | 50.80 | O |
| ATOM | 2647 | N | PHE | A | 334 | 25.088 | 3.083 | −14.143 | 1.00 | 50.97 | N |
| ATOM | 2648 | CA | PHE | A | 334 | 25.092 | 4.370 | −13.402 | 1.00 | 48.75 | C |
| ATOM | 2649 | CB | PHE | A | 334 | 25.443 | 4.129 | −11.931 | 1.00 | 44.95 | C |
| ATOM | 2650 | CG | PHE | A | 334 | 24.366 | 3.497 | −11.076 | 1.00 | 44.96 | C |
| ATOM | 2651 | CD1 | PHE | A | 334 | 23.215 | 4.189 | −10.750 | 1.00 | 46.33 | C |
| ATOM | 2652 | CE1 | PHE | A | 334 | 22.244 | 3.621 | −9.944 | 1.00 | 44.32 | C |
| ATOM | 2653 | CZ | PHE | A | 334 | 22.413 | 2.364 | −9.426 | 1.00 | 42.58 | C |
| ATOM | 2654 | CD2 | PHE | A | 334 | 24.520 | 2.229 | −10.540 | 1.00 | 45.74 | C |
| ATOM | 2655 | CE2 | PHE | A | 334 | 23.552 | 1.666 | −9.722 | 1.00 | 44.51 | C |
| ATOM | 2656 | C | PHE | A | 334 | 26.040 | 5.347 | −14.118 | 1.00 | 54.42 | C |
| ATOM | 2657 | O | PHE | A | 334 | 27.054 | 4.913 | −14.708 | 1.00 | 58.64 | O |
| ATOM | 2658 | N | THR | A | 335 | 25.716 | 6.642 | −14.081 | 1.00 | 51.38 | N |
| ATOM | 2659 | CA | THR | A | 335 | 26.474 | 7.715 | −14.768 | 1.00 | 51.10 | C |
| ATOM | 2660 | CB | THR | A | 335 | 25.551 | 8.666 | −15.551 | 1.00 | 57.85 | C |
| ATOM | 2661 | OG1 | THR | A | 335 | 24.750 | 9.445 | −14.662 | 1.00 | 56.91 | O |
| ATOM | 2662 | CG2 | THR | A | 335 | 24.639 | 7.945 | −16.522 | 1.00 | 56.45 | C |
| ATOM | 2663 | C | THR | A | 335 | 27.340 | 8.468 | −13.757 | 1.00 | 51.88 | C |
| ATOM | 2664 | O | THR | A | 335 | 28.409 | 8.923 | −14.161 | 1.00 | 59.05 | O |
| ATOM | 2665 | N | ASP | A | 336 | 26.899 | 8.625 | −12.506 | 1.00 | 51.44 | N |
| ATOM | 2666 | CA | ASP | A | 336 | 27.645 | 9.415 | −11.492 | 1.00 | 48.71 | C |
| ATOM | 2667 | CB | ASP | A | 336 | 27.081 | 10.824 | −11.333 | 1.00 | 51.98 | C |
| ATOM | 2668 | CG | ASP | A | 336 | 27.110 | 11.616 | −12.622 | 1.00 | 57.92 | C |
| ATOM | 2669 | OD1 | ASP | A | 336 | 28.203 | 12.121 | −12.973 | 1.00 | 69.54 | O |
| ATOM | 2670 | OD2 | ASP | A | 336 | 26.054 | 11.693 | −13.271 | 1.00 | 59.30 | O |
| ATOM | 2671 | C | ASP | A | 336 | 27.629 | 8.687 | −10.156 | 1.00 | 48.27 | C |
| ATOM | 2672 | O | ASP | A | 336 | 26.661 | 7.938 | −9.892 | 1.00 | 46.65 | O |
| ATOM | 2673 | N | ILE | A | 337 | 28.687 | 8.905 | −9.372 | 1.00 | 46.54 | N |
| ATOM | 2674 | CA | ILE | A | 337 | 28.835 | 8.452 | −7.964 | 1.00 | 43.87 | C |
| ATOM | 2675 | CB | ILE | A | 337 | 29.924 | 7.377 | −7.859 | 1.00 | 46.50 | C |
| ATOM | 2676 | CG1 | ILE | A | 337 | 29.611 | 6.153 | −8.717 | 1.00 | 49.17 | C |
| ATOM | 2677 | CG2 | ILE | A | 337 | 30.183 | 7.003 | −6.411 | 1.00 | 46.15 | C |
| ATOM | 2678 | CD1 | ILE | A | 337 | 28.705 | 5.149 | −8.051 | 1.00 | 54.55 | C |
| ATOM | 2679 | C | ILE | A | 337 | 29.156 | 9.679 | −7.108 | 1.00 | 44.97 | C |
| ATOM | 2680 | O | ILE | A | 337 | 29.941 | 10.542 | −7.547 | 1.00 | 45.33 | O |
| ATOM | 2681 | N | ILE | A | 338 | 28.537 | 9.762 | −5.936 | 1.00 | 46.86 | N |
| ATOM | 2682 | CA | ILE | A | 338 | 28.831 | 10.790 | −4.903 | 1.00 | 53.28 | C |
| ATOM | 2683 | CB | ILE | A | 338 | 27.550 | 11.525 | −4.448 | 1.00 | 56.93 | C |
| ATOM | 2684 | CG1 | ILE | A | 338 | 26.974 | 12.422 | −5.549 | 1.00 | 64.65 | C |
| ATOM | 2685 | CG2 | ILE | A | 338 | 27.812 | 12.314 | −3.181 | 1.00 | 58.93 | C |
| ATOM | 2686 | CD1 | ILE | A | 338 | 27.977 | 13.413 | −6.163 | 1.00 | 69.31 | C |
| ATOM | 2687 | C | ILE | A | 338 | 29.537 | 10.059 | −3.765 | 1.00 | 49.80 | C |
| ATOM | 2688 | O | ILE | A | 338 | 28.914 | 9.186 | −3.189 | 1.00 | 50.37 | O |
| ATOM | 2689 | N | LYS | A | 339 | 30.811 | 10.374 | −3.520 | 1.00 | 53.16 | N |
| ATOM | 2690 | CA | LYS | A | 339 | 31.628 | 9.825 | −2.401 | 1.00 | 51.26 | C |
| ATOM | 2691 | CB | LYS | A | 339 | 32.987 | 9.354 | −2.918 | 1.00 | 61.10 | C |
| ATOM | 2692 | CG | LYS | A | 339 | 33.813 | 8.536 | −1.941 | 1.00 | 67.73 | C |
| ATOM | 2693 | CD | LYS | A | 339 | 35.232 | 8.269 | −2.417 | 1.00 | 78.75 | C |
| ATOM | 2694 | CE | LYS | A | 339 | 36.055 | 9.509 | −2.736 | 1.00 | 83.82 | C |
| ATOM | 2695 | NZ | LYS | A | 339 | 36.253 | 10.400 | −1.566 | 1.00 | 86.69 | N |
| ATOM | 2696 | C | LYS | A | 339 | 31.770 | 10.933 | −1.369 | 1.00 | 46.60 | C |
| ATOM | 2697 | O | LYS | A | 339 | 32.217 | 12.016 | −1.727 | 1.00 | 59.56 | O |
| ATOM | 2698 | N | CYS | A | 340 | 31.339 | 10.689 | −0.147 | 1.00 | 44.17 | N |
| ATOM | 2699 | CA | CYS | A | 340 | 31.255 | 11.716 | 0.907 | 1.00 | 43.87 | C |
| ATOM | 2700 | CB | CYS | A | 340 | 29.818 | 12.098 | 1.214 | 1.00 | 47.94 | C |
| ATOM | 2701 | SG | CYS | A | 340 | 29.735 | 13.451 | 2.412 | 1.00 | 45.80 | S |
| ATOM | 2702 | C | CYS | A | 340 | 31.943 | 11.186 | 2.155 | 1.00 | 48.78 | C |
| ATOM | 2703 | O | CYS | A | 340 | 31.318 | 10.420 | 2.904 | 1.00 | 42.38 | O |
| ATOM | 2704 | N | ARG | A | 341 | 33.217 | 11.539 | 2.316 | 1.00 | 56.40 | N |
| ATOM | 2705 | CA | ARG | A | 341 | 34.050 | 11.063 | 3.443 | 1.00 | 57.13 | C |
| ATOM | 2706 | CB | ARG | A | 341 | 35.510 | 11.501 | 3.307 | 1.00 | 64.16 | C |
| ATOM | 2707 | CG | ARG | A | 341 | 36.154 | 11.162 | 1.973 | 1.00 | 72.86 | C |
| ATOM | 2708 | CD | ARG | A | 341 | 37.664 | 11.216 | 2.104 | 1.00 | 83.19 | C |
| ATOM | 2709 | NE | ARG | A | 341 | 38.159 | 12.522 | 2.537 | 1.00 | 79.01 | N |
| ATOM | 2710 | CZ | ARG | A | 341 | 38.322 | 13.583 | 1.743 | 1.00 | 77.39 | C |
| ATOM | 2711 | NH1 | ARG | A | 341 | 38.011 | 13.518 | 0.455 | 1.00 | 76.10 | N |
| ATOM | 2712 | NH2 | ARG | A | 341 | 38.788 | 14.714 | 2.254 | 1.00 | 70.19 | N |
| ATOM | 2713 | C | ARG | A | 341 | 33.476 | 11.723 | 4.676 | 1.00 | 53.32 | C |
| ATOM | 2714 | O | ARG | A | 341 | 33.187 | 12.912 | 4.572 | 1.00 | 63.45 | O |
| ATOM | 2715 | N | VAL | A | 342 | 33.309 | 11.004 | 5.775 | 1.00 | 47.28 | N |
| ATOM | 2716 | CA | VAL | A | 342 | 32.950 | 11.663 | 7.056 | 1.00 | 48.83 | C |
| ATOM | 2717 | CB | VAL | A | 342 | 32.091 | 10.778 | 7.969 | 1.00 | 47.31 | C |
| ATOM | 2718 | CG1 | VAL | A | 342 | 31.941 | 11.388 | 9.343 | 1.00 | 51.45 | C |
| ATOM | 2719 | CG2 | VAL | A | 342 | 30.722 | 10.542 | 7.370 | 1.00 | 51.79 | C |
| ATOM | 2720 | C | VAL | A | 342 | 34.262 | 12.097 | 7.712 | 1.00 | 53.67 | C |
| ATOM | 2721 | O | VAL | A | 342 | 35.254 | 11.329 | 7.631 | 1.00 | 53.71 | O |
| ATOM | 2722 | N | ILE | A | 343 | 34.264 | 13.307 | 8.270 | 1.00 | 48.60 | N |
| ATOM | 2723 | CA | ILE | A | 343 | 35.399 | 13.861 | 9.050 | 1.00 | 49.12 | C |
| ATOM | 2724 | CB | ILE | A | 343 | 35.794 | 15.252 | 8.518 | 1.00 | 52.79 | C |
| ATOM | 2725 | CG1 | ILE | A | 343 | 36.429 | 15.126 | 7.130 | 1.00 | 54.24 | C |
| ATOM | 2726 | CG2 | ILE | A | 343 | 36.704 | 15.984 | 9.496 | 1.00 | 50.81 | C |
| ATOM | 2727 | CD1 | ILE | A | 343 | 36.736 | 16.441 | 6.471 | 1.00 | 57.72 | C |
| ATOM | 2728 | C | ILE | A | 343 | 34.964 | 13.861 | 10.509 | 1.00 | 45.52 | C |
| ATOM | 2729 | O | ILE | A | 343 | 34.119 | 14.684 | 10.873 | 1.00 | 47.34 | O |
| ATOM | 2730 | N | LEU | A | 344 | 35.494 | 12.933 | 11.297 | 1.00 | 43.08 | N |
| ATOM | 2731 | CA | LEU | A | 344 | 34.988 | 12.670 | 12.662 | 1.00 | 43.44 | C |
| ATOM | 2732 | CB | LEU | A | 344 | 35.707 | 11.451 | 13.227 | 1.00 | 44.65 | C |
| ATOM | 2733 | CG | LEU | A | 344 | 35.344 | 10.123 | 12.574 | 1.00 | 48.52 | C |
| ATOM | 2734 | CD1 | LEU | A | 344 | 36.017 | 8.980 | 13.323 | 1.00 | 49.29 | C |
| ATOM | 2735 | CD2 | LEU | A | 344 | 33.831 | 9.939 | 12.545 | 1.00 | 47.96 | C |
| ATOM | 2736 | C | LEU | A | 344 | 35.216 | 13.886 | 13.548 | 1.00 | 48.06 | C |
| ATOM | 2737 | O | LEU | A | 344 | 34.465 | 14.037 | 14.526 | 1.00 | 48.74 | O |
| ATOM | 2738 | N | GLU | A | 345 | 36.248 | 14.680 | 13.251 | 1.00 | 55.09 | N |
| ATOM | 2739 | CA | GLU | A | 345 | 36.595 | 15.888 | 14.042 | 1.00 | 62.45 | C |
| ATOM | 2740 | CB | GLU | A | 345 | 37.840 | 16.589 | 13.486 | 1.00 | 69.94 | C |
| ATOM | 2741 | CG | GLU | A | 345 | 39.153 | 15.894 | 13.826 | 1.00 | 72.79 | C |
| ATOM | 2742 | CD | GLU | A | 345 | 39.393 | 14.545 | 13.161 | 1.00 | 77.33 | C |
| ATOM | 2743 | OE1 | GLU | A | 345 | 40.265 | 13.799 | 13.657 | 1.00 | 77.13 | O |
| ATOM | 2744 | OE2 | GLU | A | 345 | 38.707 | 14.235 | 12.151 | 1.00 | 75.46 | O |
| ATOM | 2745 | C | GLU | A | 345 | 35.369 | 16.799 | 14.017 | 1.00 | 61.25 | C |
| ATOM | 2746 | O | GLU | A | 345 | 34.932 | 17.229 | 15.105 | 1.00 | 63.70 | O |
| ATOM | 2747 | N | ASN | A | 346 | 34.791 | 16.986 | 12.826 | 1.00 | 60.93 | N |
| ATOM | 2748 | CA | ASN | A | 346 | 33.626 | 17.881 | 12.587 | 1.00 | 61.70 | C |
| ATOM | 2749 | CB | ASN | A | 346 | 33.437 | 18.135 | 11.089 | 1.00 | 58.16 | C |
| ATOM | 2750 | CG | ASN | A | 346 | 34.637 | 18.779 | 10.416 | 1.00 | 53.37 | C |
| ATOM | 2751 | OD1 | ASN | A | 346 | 35.417 | 19.492 | 11.040 | 1.00 | 63.23 | O |
| ATOM | 2752 | ND2 | ASN | A | 346 | 34.798 | 18.550 | 9.126 | 1.00 | 51.88 | N |
| ATOM | 2753 | C | ASN | A | 346 | 32.372 | 17.338 | 13.306 | 1.00 | 66.69 | C |
| ATOM | 2754 | O | ASN | A | 346 | 31.577 | 18.175 | 13.751 | 1.00 | 68.06 | O |
| ATOM | 2755 | N | LEU | A | 347 | 32.218 | 16.015 | 13.479 | 1.00 | 70.73 | N |
| ATOM | 2756 | CA | LEU | A | 347 | 31.064 | 15.390 | 14.203 | 1.00 | 74.47 | C |
| ATOM | 2757 | CB | LEU | A | 347 | 31.023 | 13.872 | 13.979 | 1.00 | 76.19 | C |
| ATOM | 2758 | CG | LEU | A | 347 | 30.302 | 13.362 | 12.732 | 1.00 | 76.23 | C |
| ATOM | 2759 | CD1 | LEU | A | 347 | 29.979 | 11.880 | 12.889 | 1.00 | 72.17 | C |
| ATOM | 2760 | CD2 | LEU | A | 347 | 29.032 | 14.152 | 12.436 | 1.00 | 71.77 | C |
| ATOM | 2761 | C | LEU | A | 347 | 31.131 | 15.639 | 15.713 | 1.00 | 76.21 | C |
| ATOM | 2762 | O | LEU | A | 347 | 30.052 | 15.816 | 16.297 | 1.00 | 95.38 | O |
| ATOM | 2763 | N | TYR | A | 348 | 32.317 | 15.539 | 16.326 | 1.00 | 78.72 | N |
| ATOM | 2764 | CA | TYR | A | 348 | 32.534 | 15.561 | 17.803 | 1.00 | 76.55 | C |
| ATOM | 2765 | CB | TYR | A | 348 | 33.670 | 14.580 | 18.148 | 1.00 | 76.49 | C |
| ATOM | 2766 | CG | TYR | A | 348 | 34.081 | 14.396 | 19.599 | 1.00 | 75.38 | C |
| ATOM | 2767 | CD1 | TYR | A | 348 | 33.481 | 13.453 | 20.423 | 1.00 | 75.87 | C |
| ATOM | 2768 | CE1 | TYR | A | 348 | 33.897 | 13.253 | 21.734 | 1.00 | 69.43 | C |
| ATOM | 2769 | CZ | TYR | A | 348 | 34.952 | 13.987 | 22.247 | 1.00 | 70.74 | C |
| ATOM | 2770 | OH | TYR | A | 348 | 35.380 | 13.823 | 23.538 | 1.00 | 72.08 | O |
| ATOM | 2771 | CE2 | TYR | A | 348 | 35.584 | 14.906 | 21.434 | 1.00 | 67.71 | C |
| ATOM | 2772 | CD2 | TYR | A | 348 | 35.159 | 15.090 | 20.129 | 1.00 | 72.72 | C |
| ATOM | 2773 | C | TYR | A | 348 | 32.708 | 17.029 | 18.246 | 1.00 | 82.26 | C |
| ATOM | 2774 | O | TYR | A | 348 | 32.697 | 17.299 | 19.468 | 1.00 | 79.57 | O |
| ATOM | 2775 | N | PHE | A | 349 | 32.836 | 17.954 | 17.279 | 1.00 | 94.80 | N |
| ATOM | 2776 | CA | PHE | A | 349 | 32.608 | 19.423 | 17.426 | 1.00 | 99.57 | C |
| ATOM | 2777 | CB | PHE | A | 349 | 32.187 | 20.031 | 16.083 | 1.00 | 99.44 | C |
| ATOM | 2778 | CG | PHE | A | 349 | 32.329 | 21.529 | 15.956 | 1.00 | 113.47 | C |
| ATOM | 2779 | CD1 | PHE | A | 349 | 33.557 | 22.099 | 15.641 | 1.00 | 115.61 | C |
| ATOM | 2780 | CE1 | PHE | A | 349 | 33.688 | 23.475 | 15.510 | 1.00 | 119.40 | C |
| ATOM | 2781 | CZ | PHE | A | 349 | 32.592 | 24.296 | 15.676 | 1.00 | 120.85 | C |
| ATOM | 2782 | CD2 | PHE | A | 349 | 31.231 | 22.369 | 16.103 | 1.00 | 121.28 | C |
| ATOM | 2783 | CE2 | PHE | A | 349 | 31.363 | 23.745 | 15.970 | 1.00 | 122.28 | C |
| ATOM | 2784 | C | PHE | A | 349 | 31.530 | 19.681 | 18.489 | 1.00 | 98.42 | C |
| ATOM | 2785 | O | PHE | A | 349 | 31.435 | 20.740 | 19.112 | 1.00 | 100.05 | O |
| TER | 2786 | | PHE | A | 349 | | | | | | |
| ATOM | 2787 | N | VAL | B | 5 | 26.720 | −2.739 | 19.049 | 1.00 | 89.01 | N |
| ATOM | 2788 | CA | VAL | B | 5 | 27.103 | −2.918 | 20.494 | 1.00 | 90.17 | C |
| ATOM | 2789 | CB | VAL | B | 5 | 26.443 | −1.856 | 21.398 | 1.00 | 87.95 | C |
| ATOM | 2790 | CG1 | VAL | B | 5 | 26.833 | −2.047 | 22.858 | 1.00 | 83.19 | C |
| ATOM | 2791 | CG2 | VAL | B | 5 | 26.764 | −0.440 | 20.943 | 1.00 | 83.43 | C |
| ATOM | 2792 | C | VAL | B | 5 | 26.737 | −4.336 | 20.963 | 1.00 | 85.59 | C |
| ATOM | 2793 | O | VAL | B | 5 | 25.525 | −4.655 | 21.027 | 1.00 | 82.22 | O |
| ATOM | 2794 | N | LYS | B | 6 | 27.746 | −5.132 | 21.336 | 1.00 | 85.82 | N |
| ATOM | 2795 | CA | LYS | B | 6 | 27.595 | −6.537 | 21.811 | 1.00 | 87.92 | C |
| ATOM | 2796 | CB | LYS | B | 6 | 28.972 | −7.210 | 21.834 | 1.00 | 89.72 | C |
| ATOM | 2797 | CG | LYS | B | 6 | 28.981 | −8.691 | 22.188 | 1.00 | 95.24 | C |
| ATOM | 2798 | CD | LYS | B | 6 | 28.663 | −9.609 | 21.026 | 1.00 | 97.95 | C |
| ATOM | 2799 | CE | LYS | B | 6 | 29.022 | −11.053 | 21.307 | 1.00 | 97.75 | C |
| ATOM | 2800 | NZ | LYS | B | 6 | 29.018 | −11.868 | 20.069 | 1.00 | 101.62 | N |
| ATOM | 2801 | C | LYS | B | 6 | 26.939 | −6.532 | 23.194 | 1.00 | 86.31 | C |
| ATOM | 2802 | O | LYS | B | 6 | 27.469 | −5.921 | 24.115 | 1.00 | 75.23 | O |
| ATOM | 2803 | N | PRO | B | 7 | 25.765 | −7.180 | 23.401 | 1.00 | 93.79 | N |
| ATOM | 2804 | CA | PRO | B | 7 | 25.192 | −7.318 | 24.742 | 1.00 | 88.56 | C |
| ATOM | 2805 | CB | PRO | B | 7 | 23.930 | −8.175 | 24.542 | 1.00 | 87.61 | C |
| ATOM | 2806 | CG | PRO | B | 7 | 23.573 | −7.975 | 23.089 | 1.00 | 88.24 | C |
| ATOM | 2807 | CD | PRO | B | 7 | 24.904 | −7.796 | 22.379 | 1.00 | 93.53 | C |
| ATOM | 2808 | C | PRO | B | 7 | 26.184 | −8.018 | 25.679 | 1.00 | 79.08 | C |
| ATOM | 2809 | O | PRO | B | 7 | 26.845 | −8.927 | 25.234 | 1.00 | 78.63 | O |
| ATOM | 2810 | N | TYR | B | 8 | 26.282 | −7.563 | 26.931 | 1.00 | 81.36 | N |
| ATOM | 2811 | CA | TYR | B | 8 | 27.100 | −8.218 | 27.984 | 1.00 | 73.72 | C |
| ATOM | 2812 | CB | TYR | B | 8 | 27.472 | −7.272 | 29.129 | 1.00 | 65.84 | C |
| ATOM | 2813 | CG | TYR | B | 8 | 28.574 | −7.813 | 30.004 | 1.00 | 61.74 | C |
| ATOM | 2814 | CD1 | TYR | B | 8 | 29.822 | −8.105 | 29.473 | 1.00 | 57.98 | C |
| ATOM | 2815 | CE1 | TYR | B | 8 | 30.850 | −8.601 | 30.261 | 1.00 | 60.33 | C |
| ATOM | 2816 | CZ | TYR | B | 8 | 30.634 | −8.825 | 31.613 | 1.00 | 66.54 | C |
| ATOM | 2817 | OH | TYR | B | 8 | 31.640 | −9.313 | 32.406 | 1.00 | 72.52 | O |
| ATOM | 2818 | CE2 | TYR | B | 8 | 29.391 | −8.545 | 32.159 | 1.00 | 59.47 | C |
| ATOM | 2819 | CD2 | TYR | B | 8 | 28.375 | −8.048 | 31.355 | 1.00 | 59.92 | C |
| ATOM | 2820 | C | TYR | B | 8 | 26.325 | −9.433 | 28.499 | 1.00 | 76.91 | C |
| ATOM | 2821 | O | TYR | B | 8 | 25.122 | −9.293 | 28.890 | 1.00 | 65.42 | O |
| ATOM | 2822 | N | GLU | B | 9 | 26.988 | −10.593 | 28.453 | 1.00 | 80.66 | N |
| ATOM | 2823 | CA | GLU | B | 9 | 26.430 | −11.890 | 28.900 | 1.00 | 85.01 | C |
| ATOM | 2824 | CB | GLU | B | 9 | 26.513 | −11.951 | 30.427 | 1.00 | 88.33 | C |
| ATOM | 2825 | CG | GLU | B | 9 | 27.937 | −11.767 | 30.951 | 1.00 | 94.16 | C |
| ATOM | 2826 | CD | GLU | B | 9 | 28.075 | −11.475 | 32.441 | 1.00 | 98.70 | C |
| ATOM | 2827 | OE1 | GLU | B | 9 | 29.174 | −11.715 | 33.000 | 1.00 | 92.00 | O |
| ATOM | 2828 | OE2 | GLU | B | 9 | 27.098 | −10.983 | 33.040 | 1.00 | 103.70 | O |
| ATOM | 2829 | C | GLU | B | 9 | 25.023 | −12.018 | 28.298 | 1.00 | 88.18 | C |
| ATOM | 2830 | O | GLU | B | 9 | 24.054 | −12.215 | 29.063 | 1.00 | 94.32 | O |
| ATOM | 2831 | N | ASP | B | 10 | 24.938 | −11.857 | 26.967 | 1.00 | 83.20 | N |
| ATOM | 2832 | CA | ASP | B | 10 | 23.766 | −12.194 | 26.114 | 1.00 | 83.42 | C |
| ATOM | 2833 | CB | ASP | B | 10 | 23.666 | −13.708 | 25.868 | 1.00 | 92.01 | C |
| ATOM | 2834 | CG | ASP | B | 10 | 24.979 | −14.406 | 25.533 | 1.00 | 102.39 | C |
| ATOM | 2835 | OD1 | ASP | B | 10 | 25.958 | −13.703 | 25.202 | 1.00 | 111.68 | O |
| ATOM | 2836 | OD2 | ASP | B | 10 | 25.018 | −15.657 | 25.613 | 1.00 | 109.60 | O |
| ATOM | 2837 | C | ASP | B | 10 | 22.490 | −11.690 | 26.785 | 1.00 | 76.48 | C |
| ATOM | 2838 | O | ASP | B | 10 | 21.539 | −12.473 | 26.888 | 1.00 | 83.94 | O |
| ATOM | 2839 | N | GLN | B | 11 | 22.491 | −10.450 | 27.273 | 1.00 | 74.11 | N |
| ATOM | 2840 | CA | GLN | B | 11 | 21.326 | −9.845 | 27.970 | 1.00 | 64.48 | C |
| ATOM | 2841 | CB | GLN | B | 11 | 21.685 | −9.441 | 29.401 | 1.00 | 67.18 | C |
| ATOM | 2842 | CG | GLN | B | 11 | 21.237 | −10.459 | 30.444 | 1.00 | 63.33 | C |
| ATOM | 2843 | CD | GLN | B | 11 | 21.593 | −10.056 | 31.857 | 1.00 | 61.06 | C |
| ATOM | 2844 | OE1 | GLN | B | 11 | 22.630 | −9.450 | 32.110 | 1.00 | 58.35 | O |
| ATOM | 2845 | NE2 | GLN | B | 11 | 20.751 | −10.432 | 32.808 | 1.00 | 57.90 | N |
| ATOM | 2846 | C | GLN | B | 11 | 20.815 | −8.681 | 27.125 | 1.00 | 59.74 | C |
| ATOM | 2847 | O | GLN | B | 11 | 21.572 | −7.705 | 26.910 | 1.00 | 51.89 | O |
| ATOM | 2848 | N | ASN | B | 12 | 19.574 | −8.809 | 26.656 | 1.00 | 64.52 | N |
| ATOM | 2849 | CA | ASN | B | 12 | 18.935 | −7.851 | 25.728 | 1.00 | 64.35 | C |
| ATOM | 2850 | CB | ASN | B | 12 | 18.154 | −8.572 | 24.637 | 1.00 | 71.28 | C |
| ATOM | 2851 | CG | ASN | B | 12 | 17.675 | −7.598 | 23.586 | 1.00 | 77.87 | C |
| ATOM | 2852 | OD1 | ASN | B | 12 | 16.658 | −6.936 | 23.772 | 1.00 | 82.39 | O |
| ATOM | 2853 | ND2 | ASN | B | 12 | 18.432 | −7.463 | 22.510 | 1.00 | 85.31 | N |
| ATOM | 2854 | C | ASN | B | 12 | 18.040 | −6.894 | 26.516 | 1.00 | 64.03 | C |
| ATOM | 2855 | O | ASN | B | 12 | 16.913 | −7.287 | 26.860 | 1.00 | 72.95 | O |
| ATOM | 2856 | N | TYR | B | 13 | 18.524 | −5.670 | 26.745 | 1.00 | 62.91 | N |
| ATOM | 2857 | CA | TYR | B | 13 | 17.808 | −4.573 | 27.449 | 1.00 | 65.51 | C |
| ATOM | 2858 | CB | TYR | B | 13 | 18.580 | −3.251 | 27.318 | 1.00 | 63.34 | C |
| ATOM | 2859 | CG | TYR | B | 13 | 17.833 | −2.050 | 27.848 | 1.00 | 61.89 | C |
| ATOM | 2860 | CD1 | TYR | B | 13 | 17.745 | −1.797 | 29.209 | 1.00 | 59.05 | C |
| ATOM | 2861 | CE1 | TYR | B | 13 | 17.041 | −0.713 | 29.703 | 1.00 | 52.57 | C |
| ATOM | 2862 | CZ | TYR | B | 13 | 16.410 | 0.149 | 28.831 | 1.00 | 55.52 | C |
| ATOM | 2863 | OH | TYR | B | 13 | 15.710 | 1.215 | 29.306 | 1.00 | 62.43 | O |
| ATOM | 2864 | CE2 | TYR | B | 13 | 16.476 | −0.087 | 27.473 | 1.00 | 60.21 | C |
| ATOM | 2865 | CD2 | TYR | B | 13 | 17.172 | −1.185 | 26.994 | 1.00 | 62.86 | C |
| ATOM | 2866 | C | TYR | B | 13 | 16.369 | −4.449 | 26.913 | 1.00 | 66.61 | C |
| ATOM | 2867 | O | TYR | B | 13 | 15.414 | −4.417 | 27.728 | 1.00 | 67.03 | O |
| ATOM | 2868 | N | SER | B | 14 | 16.214 | −4.373 | 25.590 | 1.00 | 65.57 | N |
| ATOM | 2869 | CA | SER | B | 14 | 14.937 | −4.039 | 24.905 | 1.00 | 75.39 | C |
| ATOM | 2870 | CB | SER | B | 14 | 15.133 | −3.867 | 23.407 | 1.00 | 82.46 | C |
| ATOM | 2871 | OG | SER | B | 14 | 16.062 | −2.826 | 23.120 | 1.00 | 82.48 | O |
| ATOM | 2872 | C | SER | B | 14 | 13.893 | −5.117 | 25.215 | 1.00 | 74.09 | C |
| ATOM | 2873 | O | SER | B | 14 | 12.761 | −4.744 | 25.604 | 1.00 | 63.90 | O |
| ATOM | 2874 | N | ALA | B | 15 | 14.265 | −6.394 | 25.044 | 1.00 | 73.65 | N |
| ATOM | 2875 | CA | ALA | B | 15 | 13.412 | −7.580 | 25.310 | 1.00 | 74.97 | C |
| ATOM | 2876 | CB | ALA | B | 15 | 14.140 | −8.844 | 24.909 | 1.00 | 76.12 | C |
| ATOM | 2877 | C | ALA | B | 15 | 13.021 | −7.609 | 26.796 | 1.00 | 73.40 | C |
| ATOM | 2878 | O | ALA | B | 15 | 11.808 | −7.661 | 27.108 | 1.00 | 67.31 | O |
| ATOM | 2879 | N | LEU | B | 16 | 14.015 | −7.560 | 27.686 | 1.00 | 64.90 | N |
| ATOM | 2880 | CA | LEU | B | 16 | 13.803 | −7.626 | 29.153 | 1.00 | 62.10 | C |
| ATOM | 2881 | CB | LEU | B | 16 | 15.166 | −7.549 | 29.838 | 1.00 | 60.41 | C |
| ATOM | 2882 | CG | LEU | B | 16 | 16.069 | −8.755 | 29.588 | 1.00 | 61.05 | C |
| ATOM | 2883 | CD1 | LEU | B | 16 | 17.480 | −8.506 | 30.110 | 1.00 | 63.13 | C |
| ATOM | 2884 | CD2 | LEU | B | 16 | 15.480 | −10.020 | 30.200 | 1.00 | 60.60 | C |
| ATOM | 2885 | C | LEU | B | 16 | 12.842 | −6.507 | 29.595 | 1.00 | 66.33 | C |
| ATOM | 2886 | O | LEU | B | 16 | 11.798 | −6.827 | 30.216 | 1.00 | 64.69 | O |
| ATOM | 2887 | N | ARG | B | 17 | 13.119 | −5.250 | 29.242 | 1.00 | 63.34 | N |
| ATOM | 2888 | CA | ARG | B | 17 | 12.236 | −4.119 | 29.622 | 1.00 | 67.20 | C |
| ATOM | 2889 | CB | ARG | B | 17 | 12.768 | −2.799 | 29.066 | 1.00 | 69.63 | C |
| ATOM | 2890 | CG | ARG | B | 17 | 11.863 | −1.618 | 29.391 | 1.00 | 68.30 | C |
| ATOM | 2891 | CD | ARG | B | 17 | 12.441 | −0.304 | 28.931 | 1.00 | 68.58 | C |
| ATOM | 2892 | NE | ARG | B | 17 | 12.605 | −0.258 | 27.488 | 1.00 | 72.81 | N |
| ATOM | 2893 | CZ | ARG | B | 17 | 13.034 | 0.804 | 26.813 | 1.00 | 71.69 | C |
| ATOM | 2894 | NH1 | ARG | B | 17 | 13.347 | 1.918 | 27.451 | 1.00 | 75.32 | N |
| ATOM | 2895 | NH2 | ARG | B | 17 | 13.148 | 0.753 | 25.499 | 1.00 | 72.07 | N |
| ATOM | 2896 | C | ARG | B | 17 | 10.802 | −4.382 | 29.132 | 1.00 | 71.85 | C |
| ATOM | 2897 | O | ARG | B | 17 | 9.858 | −4.193 | 29.926 | 1.00 | 70.25 | O |
| ATOM | 2898 | N | ARG | B | 18 | 10.638 | −4.760 | 27.863 | 1.00 | 78.93 | N |
| ATOM | 2899 | CA | ARG | B | 18 | 9.312 | −4.992 | 27.225 | 1.00 | 88.07 | C |
| ATOM | 2900 | CB | ARG | B | 18 | 9.506 | −5.337 | 25.741 | 1.00 | 100.11 | C |
| ATOM | 2901 | CG | ARG | B | 18 | 8.229 | −5.407 | 24.912 | 1.00 | 108.67 | C |
| ATOM | 2902 | CD | ARG | B | 18 | 8.493 | −5.702 | 23.440 | 1.00 | 109.70 | C |
| ATOM | 2903 | NE | ARG | B | 18 | 7.265 | −5.992 | 22.699 | 1.00 | 121.67 | N |
| ATOM | 2904 | CZ | ARG | B | 18 | 6.407 | −5.085 | 22.213 | 1.00 | 121.45 | C |
| ATOM | 2905 | NH1 | ARG | B | 18 | 6.620 | −3.788 | 22.373 | 1.00 | 118.53 | N |
| ATOM | 2906 | NH2 | ARG | B | 18 | 5.326 | −5.483 | 21.561 | 1.00 | 111.83 | N |
| ATOM | 2907 | C | ARG | B | 18 | 8.569 | −6.076 | 28.024 | 1.00 | 83.76 | C |
| ATOM | 2908 | O | ARG | B | 18 | 7.419 | −5.822 | 28.431 | 1.00 | 79.67 | O |
| ATOM | 2909 | N | ASP | B | 19 | 9.222 | −7.217 | 28.275 | 1.00 | 80.53 | N |
| ATOM | 2910 | CA | ASP | B | 19 | 8.671 | −8.354 | 29.065 | 1.00 | 88.93 | C |
| ATOM | 2911 | CB | ASP | B | 19 | 9.698 | −9.482 | 29.207 | 1.00 | 97.53 | C |
| ATOM | 2912 | CG | ASP | B | 19 | 9.195 | −10.685 | 29.988 | 1.00 | 105.74 | C |
| ATOM | 2913 | OD1 | ASP | B | 19 | 8.542 | −11.552 | 29.373 | 1.00 | 115.58 | O |
| ATOM | 2914 | OD2 | ASP | B | 19 | 9.449 | −10.740 | 31.208 | 1.00 | 113.31 | O |
| ATOM | 2915 | C | ASP | B | 19 | 8.204 | −7.871 | 30.448 | 1.00 | 91.31 | C |
| ATOM | 2916 | O | ASP | B | 19 | 7.108 | −8.288 | 30.870 | 1.00 | 96.07 | O |
| ATOM | 2917 | N | CYS | B | 20 | 9.005 | −7.042 | 31.132 | 1.00 | 87.11 | N |
| ATOM | 2918 | CA | CYS | B | 20 | 8.720 | −6.531 | 32.502 | 1.00 | 77.33 | C |
| ATOM | 2919 | CB | CYS | B | 20 | 9.930 | −5.844 | 33.131 | 1.00 | 80.66 | C |
| ATOM | 2920 | SG | CYS | B | 20 | 11.306 | −6.945 | 33.568 | 1.00 | 68.26 | S |
| ATOM | 2921 | C | CYS | B | 20 | 7.544 | −5.546 | 32.467 | 1.00 | 76.56 | C |
| ATOM | 2922 | O | CYS | B | 20 | 6.705 | −5.622 | 33.385 | 1.00 | 85.40 | O |
| ATOM | 2923 | N | ARG | B | 21 | 7.480 | −4.646 | 31.477 | 1.00 | 79.98 | N |
| ATOM | 2924 | CA | ARG | B | 21 | 6.339 | −3.695 | 31.310 | 1.00 | 83.68 | C |
| ATOM | 2925 | CB | ARG | B | 21 | 6.594 | −2.647 | 30.228 | 1.00 | 77.58 | C |
| ATOM | 2926 | CG | ARG | B | 21 | 7.653 | −1.619 | 30.585 | 1.00 | 76.57 | C |
| ATOM | 2927 | CD | ARG | B | 21 | 8.239 | −1.025 | 29.326 | 1.00 | 75.91 | C |
| ATOM | 2928 | NE | ARG | B | 21 | 8.780 | 0.296 | 29.545 | 1.00 | 74.34 | N |
| ATOM | 2929 | CZ | ARG | B | 21 | 9.405 | 1.016 | 28.625 | 1.00 | 83.34 | C |
| ATOM | 2930 | NH1 | ARG | B | 21 | 9.585 | 0.538 | 27.405 | 1.00 | 92.08 | N |
| ATOM | 2931 | NH2 | ARG | B | 21 | 9.864 | 2.216 | 28.933 | 1.00 | 87.88 | N |
| ATOM | 2932 | C | ARG | B | 21 | 5.080 | −4.460 | 30.904 | 1.00 | 86.93 | C |
| ATOM | 2933 | O | ARG | B | 21 | 3.982 | −4.020 | 31.289 | 1.00 | 95.00 | O |
| ATOM | 2934 | N | ARG | B | 22 | 5.246 | −5.527 | 30.117 | 1.00 | 91.40 | N |
| ATOM | 2935 | CA | ARG | B | 22 | 4.150 | −6.436 | 29.689 | 1.00 | 94.34 | C |
| ATOM | 2936 | CB | ARG | B | 22 | 4.726 | −7.561 | 28.817 | 1.00 | 102.12 | C |
| ATOM | 2937 | CG | ARG | B | 22 | 3.706 | −8.390 | 28.045 | 1.00 | 102.83 | C |
| ATOM | 2938 | CD | ARG | B | 22 | 3.518 | −9.755 | 28.687 | 1.00 | 104.96 | C |
| ATOM | 2939 | NE | ARG | B | 22 | 2.796 | −10.704 | 27.850 | 1.00 | 102.76 | N |
| ATOM | 2940 | CZ | ARG | B | 22 | 1.472 | −10.857 | 27.812 | 1.00 | 105.15 | C |
| ATOM | 2941 | NH1 | ARG | B | 22 | 0.675 | −10.115 | 28.567 | 1.00 | 103.93 | N |
| ATOM | 2942 | NH2 | ARG | B | 22 | 0.946 | −11.758 | 26.999 | 1.00 | 101.81 | N |
| ATOM | 2943 | C | ARG | B | 22 | 3.438 | −6.911 | 30.961 | 1.00 | 85.47 | C |
| ATOM | 2944 | O | ARG | B | 22 | 2.252 | −6.591 | 31.104 | 1.00 | 81.10 | O |
| ATOM | 2945 | N | ARG | B | 23 | 4.180 | −7.524 | 31.891 | 1.00 | 83.27 | N |
| ATOM | 2946 | CA | ARG | B | 23 | 3.650 | −8.227 | 33.094 | 1.00 | 85.04 | C |
| ATOM | 2947 | CB | ARG | B | 23 | 4.581 | −9.394 | 33.440 | 1.00 | 84.64 | C |
| ATOM | 2948 | CG | ARG | B | 23 | 4.860 | −10.347 | 32.283 | 1.00 | 84.16 | C |
| ATOM | 2949 | CD | ARG | B | 23 | 6.216 | −11.030 | 32.357 | 1.00 | 81.88 | C |
| ATOM | 2950 | NE | ARG | B | 23 | 6.615 | −11.309 | 33.733 | 1.00 | 84.37 | N |
| ATOM | 2951 | CZ | ARG | B | 23 | 7.488 | −12.236 | 34.112 | 1.00 | 80.51 | C |
| ATOM | 2952 | NH1 | ARG | B | 23 | 8.072 | −13.022 | 33.223 | 1.00 | 80.48 | N |
| ATOM | 2953 | NH2 | ARG | B | 23 | 7.773 | −12.373 | 35.395 | 1.00 | 76.40 | N |
| ATOM | 2954 | C | ARG | B | 23 | 3.532 | −7.304 | 34.323 | 1.00 | 88.93 | C |
| ATOM | 2955 | O | ARG | B | 23 | 3.268 | −7.845 | 35.419 | 1.00 | 93.42 | O |
| ATOM | 2956 | N | LYS | B | 24 | 3.716 | −5.984 | 34.181 | 1.00 | 90.02 | N |
| ATOM | 2957 | CA | LYS | | 24 | 3.751 | −5.004 | 35.309 | 1.00 | 86.71 | C |
| ATOM | 2958 | CB | LYS | B | 24 | 2.338 | −4.603 | 35.729 | 1.00 | 86.69 | C |
| ATOM | 2959 | CG | LYS | B | 24 | 1.570 | −3.804 | 34.692 | 1.00 | 91.31 | C |
| ATOM | 2960 | CD | LYS | B | 24 | 0.211 | −3.378 | 35.184 | 1.00 | 93.37 | C |
| ATOM | 2961 | CE | LYS | B | 24 | −0.621 | −2.717 | 34.106 | 1.00 | 95.13 | C |
| ATOM | 2962 | NZ | LYS | B | 24 | −2.042 | −3.125 | 34.194 | 1.00 | 94.57 | N |
| ATOM | 2963 | C | LYS | B | 24 | 4.493 | −5.589 | 36.518 | 1.00 | 84.80 | C |
| ATOM | 2964 | O | LYS | B | 24 | 3.940 | −5.568 | 37.626 | 1.00 | 78.96 | O |
| ATOM | 2965 | N | VAL | B | 25 | 5.705 | −6.092 | 36.290 | 1.00 | 82.71 | N |
| ATOM | 2966 | CA | VAL | B | 25 | 6.676 | −6.535 | 37.334 | 1.00 | 81.74 | C |
| ATOM | 2967 | CB | VAL | B | 25 | 7.038 | −8.019 | 37.121 | 1.00 | 83.67 | C |
| ATOM | 2968 | CG1 | VAL | B | 25 | 7.902 | −8.206 | 35.877 | 1.00 | 86.34 | C |
| ATOM | 2969 | CG2 | VAL | B | 25 | 7.700 | −8.652 | 38.342 | 1.00 | 81.04 | C |
| ATOM | 2970 | C | VAL | B | 25 | 7.903 | −5.608 | 37.238 | 1.00 | 84.72 | C |
| ATOM | 2971 | O | VAL | B | 25 | 7.946 | −4.777 | 36.287 | 1.00 | 79.58 | O |
| ATOM | 2972 | N | LEU | B | 26 | 8.862 | −5.715 | 38.168 | 1.00 | 70.90 | N |
| ATOM | 2973 | CA | LEU | B | 26 | 10.135 | −4.942 | 38.104 | 1.00 | 64.44 | C |
| ATOM | 2974 | CB | LEU | B | 26 | 10.321 | −4.128 | 39.390 | 1.00 | 62.08 | C |
| ATOM | 2975 | CG | LEU | B | 26 | 9.309 | −2.996 | 39.550 | 1.00 | 65.43 | C |
| ATOM | 2976 | CD1 | LEU | B | 26 | 9.546 | −2.198 | 40.828 | 1.00 | 64.34 | C |
| ATOM | 2977 | CD2 | LEU | B | 26 | 9.330 | −2.093 | 38.320 | 1.00 | 65.58 | C |
| ATOM | 2978 | C | LEU | B | 26 | 11.295 | −5.900 | 37.841 | 1.00 | 56.92 | C |
| ATOM | 2979 | O | LEU | B | 26 | 11.378 | −6.936 | 38.508 | 1.00 | 58.61 | O |
| ATOM | 2980 | N | PHE | B | 27 | 12.147 | −5.571 | 36.875 | 1.00 | 49.88 | N |
| ATOM | 2981 | CA | PHE | B | 27 | 13.267 | −6.448 | 36.456 | 1.00 | 55.03 | C |
| ATOM | 2982 | CB | PHE | B | 27 | 14.111 | −5.726 | 35.409 | 1.00 | 49.54 | C |
| ATOM | 2983 | CG | PHE | B | 27 | 15.354 | −6.484 | 35.037 | 1.00 | 47.54 | C |
| ATOM | 2984 | CD1 | PHE | B | 27 | 15.259 | −7.756 | 34.504 | 1.00 | 47.76 | C |
| ATOM | 2985 | CE1 | PHE | B | 27 | 16.398 | −8.462 | 34.165 | 1.00 | 44.95 | C |
| ATOM | 2986 | CZ | PHE | B | 27 | 17.634 | −7.905 | 34.365 | 1.00 | 46.93 | C |
| ATOM | 2987 | CD2 | PHE | B | 27 | 16.606 | −5.938 | 35.242 | 1.00 | 44.61 | C |
| ATOM | 2988 | CE2 | PHE | B | 27 | 17.743 | −6.650 | 34.908 | 1.00 | 46.63 | C |
| ATOM | 2989 | C | PHE | B | 27 | 14.101 | −6.880 | 37.680 | 1.00 | 55.49 | C |
| ATOM | 2990 | O | PHE | B | 27 | 14.375 | −6.041 | 38.545 | 1.00 | 53.86 | O |
| ATOM | 2991 | N | GLU | B | 28 | 14.519 | −8.153 | 37.722 | 1.00 | 62.27 | N |
| ATOM | 2992 | CA | GLU | B | 28 | 15.415 | −8.726 | 38.766 | 1.00 | 61.94 | C |
| ATOM | 2993 | CB | GLU | B | 28 | 14.655 | −9.663 | 39.710 | 1.00 | 64.37 | C |
| ATOM | 2994 | CG | GLU | B | 28 | 13.354 | −9.077 | 40.243 | 1.00 | 68.39 | C |
| ATOM | 2995 | CD | GLU | B | 28 | 12.620 | −9.896 | 41.301 | 1.00 | 74.67 | C |
| ATOM | 2996 | OE1 | GLU | B | 28 | 11.373 | −9.783 | 41.376 | 1.00 | 87.18 | O |
| ATOM | 2997 | OE2 | GLU | B | 28 | 13.283 | −10.647 | 42.048 | 1.00 | 71.69 | O |
| ATOM | 2998 | C | GLU | B | 28 | 16.541 | −9.470 | 38.051 | 1.00 | 60.27 | C |
| ATOM | 2999 | O | GLU | B | 28 | 16.282 | −10.569 | 37.540 | 1.00 | 66.43 | O |
| ATOM | 3000 | N | ASP | B | 29 | 17.726 | −8.863 | 37.993 | 1.00 | 60.81 | N |
| ATOM | 3001 | CA | ASP | B | 29 | 18.933 | −9.396 | 37.304 | 1.00 | 60.60 | C |
| ATOM | 3002 | CB | ASP | B | 29 | 20.099 | −8.399 | 37.362 | 1.00 | 60.80 | C |
| ATOM | 3003 | CG | ASP | B | 29 | 21.227 | −8.776 | 36.418 | 1.00 | 57.11 | C |
| ATOM | 3004 | OD1 | ASP | B | 29 | 21.029 | −9.755 | 35.688 | 1.00 | 59.61 | O |
| ATOM | 3005 | OD2 | ASP | B | 29 | 22.290 | −8.113 | 36.430 | 1.00 | 46.44 | O |
| ATOM | 3006 | C | ASP | B | 29 | 19.342 | −10.736 | 37.919 | 1.00 | 61.54 | C |
| ATOM | 3007 | O | ASP | B | 29 | 19.649 | −10.798 | 39.104 | 1.00 | 66.69 | O |
| ATOM | 3008 | N | PRO | B | 30 | 19.331 | −11.852 | 37.147 | 1.00 | 62.94 | N |
| ATOM | 3009 | CA | PRO | B | 30 | 19.937 | −13.119 | 37.577 | 1.00 | 58.61 | C |
| ATOM | 3010 | CB | PRO | B | 30 | 19.448 | −14.127 | 36.527 | 1.00 | 59.67 | C |
| ATOM | 3011 | CG | PRO | B | 30 | 18.245 | −13.461 | 35.902 | 1.00 | 59.83 | C |
| ATOM | 3012 | CD | PRO | B | 30 | 18.607 | −11.997 | 35.876 | 1.00 | 59.05 | C |
| ATOM | 3013 | C | PRO | B | 30 | 21.470 | −13.235 | 37.597 | 1.00 | 56.24 | C |
| ATOM | 3014 | O | PRO | E | 30 | 21.965 | −14.078 | 38.315 | 1.00 | 58.46 | O |
| ATOM | 3015 | N | LEU | B | 31 | 22.190 | −12.460 | 36.786 | 1.00 | 57.09 | N |
| ATOM | 3016 | CA | LEU | B | 31 | 23.663 | −12.622 | 36.622 | 1.00 | 63.97 | C |
| ATOM | 3017 | CB | LEU | B | 31 | 24.069 | −12.254 | 35.194 | 1.00 | 68.20 | C |
| ATOM | 3018 | CG | LEU | B | 31 | 23.831 | −13.337 | 34.140 | 1.00 | 78.17 | C |
| ATOM | 3019 | CD1 | LEU | B | 31 | 22.353 | −13.692 | 34.007 | 1.00 | 74.08 | C |
| ATOM | 3020 | CD2 | LEU | B | 31 | 24.385 | −12.882 | 32.798 | 1.00 | 82.78 | C |
| ATOM | 3021 | C | LEU | B | 31 | 24.399 | −11.745 | 37.634 | 1.00 | 62.90 | C |
| ATOM | 3022 | O | LEU | B | 31 | 25.606 | −11.965 | 37.848 | 1.00 | 58.79 | O |
| ATOM | 3023 | N | PHE | B | 32 | 23.701 | −10.760 | 38.197 | 1.00 | 66.51 | N |
| ATOM | 3024 | CA | PHE | B | 32 | 24.225 | −9.853 | 39.250 | 1.00 | 63.63 | C |
| ATOM | 3025 | CB | PHE | B | 32 | 24.773 | −8.580 | 38.612 | 1.00 | 58.05 | C |
| ATOM | 3026 | CG | PHE | B | 32 | 25.717 | −7.815 | 39.492 | 1.00 | 49.33 | C |
| ATOM | 3027 | CD1 | PHE | B | 32 | 26.954 | −8.346 | 39.812 | 1.00 | 44.63 | C |
| ATOM | 3028 | CE1 | PHE | B | 32 | 27.840 | −7.640 | 40.613 | 1.00 | 43.28 | C |
| ATOM | 3029 | CZ | PHE | B | 32 | 27.499 | −6.391 | 41.083 | 1.00 | 40.45 | C |
| ATOM | 3030 | CD2 | PHE | B | 32 | 25.378 | −6.562 | 39.980 | 1.00 | 49.06 | C |
| ATOM | 3031 | CE2 | PHE | B | 32 | 26.270 | −5.855 | 40.772 | 1.00 | 42.80 | C |
| ATOM | 3032 | C | PHE | B | 32 | 23.108 | −9.547 | 40.236 | 1.00 | 60.13 | C |
| ATOM | 3033 | O | PHE | B | 32 | 22.528 | −8.469 | 40.201 | 1.00 | 67.60 | O |
| ATOM | 3034 | N | PRO | B | 33 | 22.759 | −10.505 | 41.121 | 1.00 | 64.49 | N |
| ATOM | 3035 | CA | PRO | B | 33 | 21.571 | −10.366 | 41.967 | 1.00 | 61.27 | C |
| ATOM | 3036 | CB | PRO | B | 33 | 21.274 | −11.837 | 42.325 | 1.00 | 58.90 | C |
| ATOM | 3037 | CG | PRO | B | 33 | 22.659 | −12.476 | 42.412 | 1.00 | 59.22 | C |
| ATOM | 3038 | CD | PRO | B | 33 | 23.492 | −11.767 | 41.360 | 1.00 | 60.71 | C |
| ATOM | 3039 | C | PRO | B | 33 | 21.800 | −9.481 | 43.211 | 1.00 | 55.09 | C |
| ATOM | 3040 | O | PRO | B | 33 | 22.924 | −9.138 | 43.542 | 1.00 | 45.21 | O |
| ATOM | 3041 | N | ALA | B | 34 | 20.703 | −9.134 | 43.879 | 1.00 | 54.51 | N |
| ATOM | 3042 | CA | ALA | B | 34 | 20.652 | −8.275 | 45.082 | 1.00 | 52.88 | C |
| ATOM | 3043 | CB | ALA | B | 34 | 19.237 | −7.768 | 45.250 | 1.00 | 55.83 | C |
| ATOM | 3044 | C | ALA | B | 34 | 21.105 | −9.063 | 46.315 | 1.00 | 52.59 | C |
| ATOM | 3045 | O | ALA | B | 34 | 20.280 | −9.239 | 47.251 | 1.00 | 61.76 | O |
| ATOM | 3046 | N | THR | B | 35 | 22.360 | −9.517 | 46.331 | 1.00 | 47.10 | N |
| ATOM | 3047 | CA | THR | B | 35 | 22.892 | −10.436 | 47.372 | 1.00 | 48.48 | C |
| ATOM | 3048 | CB | THR | B | 35 | 22.785 | −11.935 | 47.030 | 1.00 | 54.79 | C |
| ATOM | 3049 | OG1 | THR | B | 35 | 23.948 | −12.341 | 46.288 | 1.00 | 57.80 | O |
| ATOM | 3050 | CG2 | THR | B | 35 | 21.512 | −12.307 | 46.292 | 1.00 | 51.06 | C |
| ATOM | 3051 | C | THR | B | 35 | 24.362 | −10.117 | 47.542 | 1.00 | 46.28 | C |
| ATOM | 3052 | O | THR | B | 35 | 24.884 | −9.389 | 46.700 | 1.00 | 47.54 | O |
| ATOM | 3053 | N | ASP | B | 36 | 24.981 | −10.718 | 48.549 | 1.00 | 47.55 | N |
| ATOM | 3054 | CA | ASP | B | 36 | 26.360 | −10.423 | 48.994 | 1.00 | 53.97 | C |
| ATOM | 3055 | CB | ASP | B | 36 | 26.618 | −11.012 | 50.385 | 1.00 | 61.91 | C |
| ATOM | 3056 | CG | ASP | B | 36 | 25.701 | −10.452 | 51.458 | 1.00 | 62.12 | C |
| ATOM | 3057 | OD1 | ASP | B | 36 | 25.312 | −9.272 | 51.326 | 1.00 | 59.50 | O |
| ATOM | 3058 | OD2 | ASP | B | 36 | 25.358 | −11.208 | 52.390 | 1.00 | 62.53 | O |
| ATOM | 3059 | C | ASP | B | 36 | 27.354 | −10.968 | 47.973 | 1.00 | 56.59 | C |
| ATOM | 3060 | O | ASP | B | 36 | 28.532 | −10.601 | 48.080 | 1.00 | 55.12 | O |
| ATOM | 3061 | N | ASP | B | 37 | 26.923 | −11.821 | 47.035 | 1.00 | 63.58 | N |
| ATOM | 3062 | CA | ASP | B | 37 | 27.834 | −12.321 | 45.967 | 1.00 | 65.36 | C |
| ATOM | 3063 | CB | ASP | B | 37 | 27.284 | −13.577 | 45.295 | 1.00 | 71.97 | C |
| ATOM | 3064 | CG | ASP | B | 37 | 27.460 | −14.760 | 46.227 | 1.00 | 76.72 | C |
| ATOM | 3065 | OD1 | ASP | B | 37 | 28.618 | −14.954 | 46.699 | 1.00 | 65.19 | O |
| ATOM | 3066 | OD2 | ASP | B | 37 | 26.433 | −15.407 | 46.551 | 1.00 | 72.62 | O |
| ATOM | 3067 | C | ASP | B | 37 | 28.168 | −11.178 | 45.004 | 1.00 | 57.59 | C |
| ATOM | 3068 | O | ASP | B | 37 | 29.263 | −11.220 | 44.436 | 1.00 | 51.39 | O |
| ATOM | 3069 | N | SER | B | 38 | 27.280 | −10.185 | 44.887 | 1.00 | 54.67 | N |
| ATOM | 3070 | CA | SER | B | 38 | 27.442 | −8.963 | 44.059 | 1.00 | 48.01 | C |
| ATOM | 3071 | CB | SER | B | 38 | 26.111 | −8.368 | 43.745 | 1.00 | 47.22 | C |
| ATOM | 3072 | OG | SER | B | 38 | 25.194 | −9.369 | 43.337 | 1.00 | 45.07 | O |
| ATOM | 3073 | C | SER | B | 38 | 28.327 | −7.932 | 44.768 | 1.00 | 52.87 | C |
| ATOM | 3074 | O | SER | B | 38 | 28.794 | −7.002 | 44.077 | 1.00 | 54.85 | O |
| ATOM | 3075 | N | LEU | B | 39 | 28.557 | −8.069 | 46.080 | 1.00 | 51.86 | N |
| ATOM | 3076 | CA | LEU | B | 39 | 29.278 | −7.050 | 46.887 | 1.00 | 46.66 | C |
| ATOM | 3077 | CB | LEU | B | 39 | 28.490 | −6.750 | 48.160 | 1.00 | 49.34 | C |
| ATOM | 3078 | CG | LEU | B | 39 | 27.014 | −6.427 | 48.003 | 1.00 | 52.04 | C |
| ATOM | 3079 | CD1 | LEU | B | 39 | 26.416 | −6.200 | 49.385 | 1.00 | 55.65 | C |
| ATOM | 3080 | CD2 | LEU | B | 39 | 26.810 | −5.206 | 47.112 | 1.00 | 53.58 | C |
| ATOM | 3081 | C | LEU | B | 39 | 30.669 | −7.538 | 47.288 | 1.00 | 49.45 | C |
| ATOM | 3082 | O | LEU | B | 39 | 31.609 | −6.724 | 47.272 | 1.00 | 52.24 | O |
| ATOM | 3083 | N | TYR | B | 40 | 30.805 | −8.763 | 47.779 | 1.00 | 50.00 | N |
| ATOM | 3084 | CA | TYR | B | 40 | 32.093 | −9.174 | 48.391 | 1.00 | 54.90 | C |
| ATOM | 3085 | CB | TYR | B | 40 | 31.980 | −9.303 | 49.912 | 1.00 | 56.42 | C |
| ATOM | 3086 | CG | TYR | B | 40 | 31.094 | −8.282 | 50.585 | 1.00 | 62.03 | C |
| ATOM | 3087 | CD1 | TYR | B | 40 | 31.527 | −6.980 | 50.818 | 1.00 | 57.26 | C |
| ATOM | 3088 | CE1 | TYR | B | 40 | 30.716 | −6.056 | 51.459 | 1.00 | 57.03 | C |
| ATOM | 3089 | CZ | TYR | B | 40 | 29.435 | −6.415 | 51.859 | 1.00 | 60.73 | C |
| ATOM | 3090 | OH | TYR | B | 40 | 28.601 | −5.513 | 52.466 | 1.00 | 51.95 | O |
| ATOM | 3091 | CE2 | TYR | B | 40 | 28.986 | −7.708 | 51.631 | 1.00 | 62.11 | C |
| ATOM | 3092 | CD2 | TYR | B | 40 | 29.816 | −8.628 | 51.008 | 1.00 | 61.67 | C |
| ATOM | 3093 | C | TYR | B | 40 | 32.594 | −10.457 | 47.731 | 1.00 | 52.85 | C |
| ATOM | 3094 | O | TYR | B | 40 | 31.829 | −11.175 | 47.087 | 1.00 | 53.26 | O |
| ATOM | 3095 | N | TYR | B | 41 | 33.889 | −10.685 | 47.873 | 1.00 | 53.32 | N |
| ATOM | 3096 | CA | TYR | B | 41 | 34.592 | −11.937 | 47.510 | 1.00 | 65.13 | C |
| ATOM | 3097 | CB | TYR | B | 41 | 36.087 | −11.725 | 47.762 | 1.00 | 65.15 | C |
| ATOM | 3098 | CG | TYR | B | 41 | 36.755 | −10.780 | 46.790 | 1.00 | 68.33 | C |
| ATOM | 3099 | CD1 | TYR | B | 41 | 36.419 | −10.792 | 45.441 | 1.00 | 70.94 | C |
| ATOM | 3100 | CE1 | TYR | B | 41 | 37.034 | −9.949 | 44.528 | 1.00 | 71.05 | C |
| ATOM | 3101 | CZ | TYR | B | 41 | 38.035 | −9.092 | 44.948 | 1.00 | 65.26 | C |
| ATOM | 3102 | OH | TYR | B | 41 | 38.638 | −8.297 | 44.022 | 1.00 | 56.95 | O |
| ATOM | 3103 | CE2 | TYR | B | 41 | 38.397 | −9.069 | 46.287 | 1.00 | 67.57 | C |
| ATOM | 3104 | CD2 | TYR | B | 41 | 37.766 | −9.915 | 47.192 | 1.00 | 67.04 | C |
| ATOM | 3105 | C | TYR | B | 41 | 33.995 | −13.122 | 48.289 | 1.00 | 66.46 | C |
| ATOM | 3106 | O | TYR | B | 41 | 33.481 | −12.903 | 49.407 | 1.00 | 74.02 | O |
| ATOM | 3107 | N | LYS | B | 42 | 34.053 | −14.334 | 47.709 | 1.00 | 70.08 | N |
| ATOM | 3108 | CA | LYS | B | 42 | 33.506 | −15.595 | 48.293 | 1.00 | 68.18 | C |
| ATOM | 3109 | CB | LYS | B | 42 | 33.959 | −16.826 | 47.497 | 1.00 | 66.35 | C |
| ATOM | 3110 | C | LYS | B | 42 | 33.973 | −15.702 | 49.751 | 1.00 | 73.88 | C |
| ATOM | 3111 | O | LYS | B | 42 | 35.215 | −15.571 | 49.989 | 1.00 | 66.78 | O |
| ATOM | 3112 | N | GLY | B | 43 | 33.021 | −15.871 | 50.682 | 1.00 | 70.50 | N |
| ATOM | 3113 | CA | GLY | B | 43 | 33.273 | −16.007 | 52.133 | 1.00 | 79.34 | C |
| ATOM | 3114 | C | GLY | B | 43 | 34.099 | −14.862 | 52.718 | 1.00 | 83.52 | C |
| ATOM | 3115 | O | GLY | B | 43 | 34.913 | −15.121 | 53.622 | 1.00 | 87.96 | O |
| ATOM | 3116 | N | THR | B | 44 | 33.900 | −13.626 | 52.258 | 1.00 | 91.17 | N |
| ATOM | 3117 | CA | THR | B | 44 | 34.456 | −12.402 | 52.902 | 1.00 | 87.80 | C |
| ATOM | 3118 | CB | THR | B | 44 | 35.111 | −11.503 | 51.841 | 1.00 | 90.22 | C |
| ATOM | 3119 | OG1 | THR | B | 44 | 36.196 | −12.240 | 51.270 | 1.00 | 85.32 | O |
| ATOM | 3120 | CG2 | THR | B | 44 | 35.632 | −10.192 | 52.391 | 1.00 | 90.10 | C |
| ATOM | 3121 | C | THR | B | 44 | 33.337 | −11.794 | 53.755 | 1.00 | 78.50 | C |
| ATOM | 3122 | O | THR | B | 44 | 32.223 | −11.598 | 53.273 | 1.00 | 60.55 | O |
| ATOM | 3123 | N | PRO | B | 45 | 33.570 | −11.542 | 55.067 | 1.00 | 86.09 | N |
| ATOM | 3124 | CA | PRO | B | 45 | 32.540 | −10.993 | 55.948 | 1.00 | 82.21 | C |
| ATOM | 3125 | CB | PRO | B | 45 | 33.082 | −11.213 | 57.361 | 1.00 | 77.88 | C |
| ATOM | 3126 | CG | PRO | B | 45 | 34.586 | −11.105 | 57.160 | 1.00 | 82.92 | C |
| ATOM | 3127 | CD | PRO | B | 45 | 34.857 | −11.673 | 55.772 | 1.00 | 91.08 | C |
| ATOM | 3128 | C | PRO | B | 45 | 32.443 | −9.501 | 55.625 | 1.00 | 80.82 | C |
| ATOM | 3129 | O | PRO | B | 45 | 33.460 | −8.803 | 55.675 | 1.00 | 78.46 | O |
| ATOM | 3130 | N | GLY | B | 46 | 31.260 | −9.079 | 55.209 | 1.00 | 66.86 | N |
| ATOM | 3131 | CA | GLY | B | 46 | 31.014 | −7.693 | 54.800 | 1.00 | 65.61 | C |
| ATOM | 3132 | C | GLY | B | 46 | 29.801 | −7.147 | 55.535 | 1.00 | 63.06 | C |
| ATOM | 3133 | O | GLY | B | 46 | 28.924 | −7.913 | 55.932 | 1.00 | 66.13 | O |
| ATOM | 3134 | N | PRO | B | 47 | 29.718 | −5.814 | 55.726 | 1.00 | 57.85 | N |
| ATOM | 3135 | CA | PRO | B | 47 | 28.527 | −5.194 | 56.299 | 1.00 | 57.54 | C |
| ATOM | 3136 | CB | PRO | B | 47 | 28.645 | −3.743 | 55.807 | 1.00 | 58.50 | C |
| ATOM | 3137 | CG | PRO | B | 47 | 30.149 | −3.491 | 55.722 | 1.00 | 59.01 | C |
| ATOM | 3138 | CD | PRO | B | 47 | 30.763 | −4.832 | 55.384 | 1.00 | 57.87 | C |
| ATOM | 3139 | C | PRO | B | 47 | 27.199 | −5.796 | 55.817 | 1.00 | 51.40 | C |
| ATOM | 3140 | O | PRO | B | 47 | 27.011 | −5.993 | 54.653 | 1.00 | 59.71 | O |
| ATOM | 3141 | N | ALA | B | 48 | 26.269 | −6.033 | 56.728 | 1.00 | 56.15 | N |
| ATOM | 3142 | CA | ALA | B | 48 | 24.875 | −6.340 | 56.364 | 1.00 | 53.91 | C |
| ATOM | 3143 | CB | ALA | B | 48 | 24.054 | −6.700 | 57.566 | 1.00 | 56.77 | C |
| ATOM | 3144 | C | ALA | B | 48 | 24.304 | −5.115 | 55.655 | 1.00 | 50.85 | C |
| ATOM | 3145 | O | ALA | B | 48 | 24.588 | −3.957 | 56.057 | 1.00 | 49.00 | O |
| ATOM | 3146 | N | VAL | B | 49 | 23.499 | −5.382 | 54.645 | 1.00 | 45.81 | N |
| ATOM | 3147 | CA | VAL | B | 49 | 22.979 | −4.341 | 53.733 | 1.00 | 48.38 | C |
| ATOM | 3148 | CB | VAL | B | 49 | 23.818 | −4.365 | 52.441 | 1.00 | 48.75 | C |
| ATOM | 3149 | CG1 | VAL | B | 49 | 23.032 | −3.893 | 51.233 | 1.00 | 49.39 | C |
| ATOM | 3150 | CG2 | VAL | B | 49 | 25.120 | −3.589 | 52.625 | 1.00 | 42.75 | C |
| ATOM | 3151 | C | VAL | B | 49 | 21.502 | −4.657 | 53.532 | 1.00 | 49.21 | C |
| ATOM | 3152 | O | VAL | B | 49 | 21.139 | −5.828 | 53.733 | 1.00 | 52.41 | O |
| ATOM | 3153 | N | ARG | B | 50 | 20.688 | −3.651 | 53.223 | 1.00 | 47.13 | N |
| ATOM | 3154 | CA | ARG | B | 50 | 19.346 | −3.856 | 52.621 | 1.00 | 50.64 | C |
| ATOM | 3155 | CB | ARG | B | 50 | 18.231 | −3.158 | 53.409 | 1.00 | 53.56 | C |
| ATOM | 3156 | CG | ARG | B | 50 | 18.129 | −3.523 | 54.881 | 1.00 | 56.11 | C |
| ATOM | 3157 | CD | ARG | B | 50 | 16.813 | −3.084 | 55.509 | 1.00 | 59.36 | C |
| ATOM | 3158 | NE | ARG | B | 50 | 16.781 | −3.485 | 56.906 | 1.00 | 60.96 | N |
| ATOM | 3159 | CZ | ARG | B | 50 | 16.714 | −2.674 | 57.962 | 1.00 | 66.71 | C |
| ATOM | 3160 | NH1 | ARG | B | 50 | 16.614 | −1.365 | 57.822 | 1.00 | 71.54 | N |
| ATOM | 3161 | NH2 | ARG | B | 50 | 16.717 | −3.182 | 59.180 | 1.00 | 62.83 | N |
| ATOM | 3162 | C | ARG | B | 50 | 19.424 | −3.332 | 51.180 | 1.00 | 51.39 | C |
| ATOM | 3163 | O | ARG | B | 50 | 19.907 | −2.192 | 50.981 | 1.00 | 52.49 | O |
| ATOM | 3164 | N | TRP | B | 51 | 19.041 | −4.158 | 50.206 | 1.00 | 51.26 | N |
| ATOM | 3165 | CA | TRP | B | 51 | 19.018 | −3.771 | 48.776 | 1.00 | 46.21 | C |
| ATOM | 3166 | CB | TRP | B | 51 | 19.148 | −4.981 | 47.848 | 1.00 | 46.00 | C |
| ATOM | 3167 | CG | TRP | B | 51 | 20.537 | −5.538 | 47.829 | 1.00 | 44.20 | C |
| ATOM | 3168 | CD1 | TRP | B | 51 | 21.077 | −6.393 | 48.739 | 1.00 | 46.93 | C |
| ATOM | 3169 | NE1 | TRP | B | 51 | 22.366 | −6.701 | 48.401 | 1.00 | 48.60 | N |
| ATOM | 3170 | CE2 | TRP | B | 51 | 22.697 | −6.040 | 47.256 | 1.00 | 48.06 | C |
| ATOM | 3171 | CD2 | TRP | B | 51 | 21.563 | −5.309 | 46.846 | 1.00 | 49.70 | C |
| ATOM | 3172 | CE3 | TRP | B | 51 | 21.642 | −4.564 | 45.660 | 1.00 | 52.85 | C |
| ATOM | 3173 | CZ3 | TRP | B | 51 | 22.824 | −4.558 | 44.949 | 1.00 | 48.68 | C |
| ATOM | 3174 | CH2 | TRP | B | 51 | 23.931 | −5.288 | 45.383 | 1.00 | 49.43 | C |
| ATOM | 3175 | CZ2 | TRP | B | 51 | 23.885 | −6.043 | 46.532 | 1.00 | 52.07 | C |
| ATOM | 3176 | C | TRP | B | 51 | 17.722 | −3.008 | 48.592 | 1.00 | 47.19 | C |
| ATOM | 3177 | O | TRP | B | 51 | 16.710 | −3.508 | 49.097 | 1.00 | 48.04 | O |
| ATOM | 3178 | N | LYS | B | 52 | 17.782 | −1.801 | 48.021 | 1.00 | 47.83 | N |
| ATOM | 3179 | CA | LYS | B | 52 | 16.572 | −0.969 | 47.819 | 1.00 | 50.54 | C |
| ATOM | 3180 | CB | LYS | B | 52 | 16.495 | 0.119 | 48.892 | 1.00 | 51.65 | C |
| ATOM | 3181 | CG | LYS | B | 52 | 16.270 | −0.405 | 50.309 | 1.00 | 56.90 | C |
| ATOM | 3182 | CD | LYS | B | 52 | 15.906 | 0.642 | 51.355 | 1.00 | 56.32 | C |
| ATOM | 3183 | CE | LYS | B | 52 | 14.414 | 0.895 | 51.458 | 1.00 | 62.49 | C |
| ATOM | 3184 | NZ | LYS | B | 52 | 14.117 | 2.038 | 52.359 | 1.00 | 73.51 | N |
| ATOM | 3185 | C | LYS | B | 52 | 16.563 | −0.411 | 46.393 | 1.00 | 52.97 | C |
| ATOM | 3186 | O | LYS | B | 52 | 17.652 | −0.117 | 45.830 | 1.00 | 53.23 | O |
| ATOM | 3187 | N | ARG | B | 53 | 15.366 | −0.322 | 45.827 | 1.00 | 52.22 | N |
| ATOM | 3188 | CA | ARG | B | 53 | 15.090 | 0.449 | 44.595 | 1.00 | 60.21 | C |
| ATOM | 3189 | CB | ARG | B | 53 | 13.756 | 0.006 | 43.980 | 1.00 | 61.19 | C |
| ATOM | 3190 | CG | ARG | B | 53 | 13.779 | −1.450 | 43.534 | 1.00 | 60.85 | C |
| ATOM | 3191 | CD | ARG | B | 53 | 12.819 | −1.795 | 42.414 | 1.00 | 64.38 | C |
| ATOM | 3192 | NE | ARG | B | 53 | 12.867 | −3.229 | 42.165 | 1.00 | 69.48 | N |
| ATOM | 3193 | CZ | ARG | B | 53 | 13.658 | −3.837 | 41.284 | 1.00 | 73.26 | C |
| ATOM | 3194 | NH1 | ARG | B | 53 | 14.475 | −3.146 | 40.500 | 1.00 | 71.68 | N |
| ATOM | 3195 | NH2 | ARG | B | 53 | 13.596 | −5.153 | 41.175 | 1.00 | 71.28 | N |
| ATOM | 3196 | C | ARG | B | 53 | 15.115 | 1.921 | 44.976 | 1.00 | 57.65 | C |
| ATOM | 3197 | O | ARG | B | 53 | 14.827 | 2.251 | 46.126 | 1.00 | 62.26 | O |
| ATOM | 3198 | N | PRO | B | 54 | 15.467 | 2.829 | 44.034 | 1.00 | 63.70 | N |
| ATOM | 3199 | CA | PRO | B | 54 | 15.517 | 4.271 | 44.302 | 1.00 | 58.91 | C |
| ATOM | 3200 | CB | PRO | B | 54 | 15.769 | 4.868 | 42.909 | 1.00 | 60.37 | C |
| ATOM | 3201 | CG | PRO | B | 54 | 16.534 | 3.789 | 42.192 | 1.00 | 64.11 | C |
| ATOM | 3202 | CD | PRO | B | 54 | 15.853 | 2.514 | 42.648 | 1.00 | 66.00 | C |
| ATOM | 3203 | C | PRO | B | 54 | 14.237 | 4.838 | 44.947 | 1.00 | 56.37 | C |
| ATOM | 3204 | O | PRO | B | 54 | 14.389 | 5.565 | 45.899 | 1.00 | 56.97 | O |
| ATOM | 3205 | N | LYS | B | 55 | 13.040 | 4.453 | 44.481 | 1.00 | 55.27 | N |
| ATOM | 3206 | CA | LYS | B | 55 | 11.727 | 4.924 | 45.017 | 1.00 | 64.90 | C |
| ATOM | 3207 | CB | LYS | B | 55 | 10.563 | 4.351 | 44.199 | 1.00 | 71.82 | C |
| ATOM | 3208 | CG | LYS | B | 55 | 9.179 | 4.480 | 44.826 | 1.00 | 82.99 | C |
| ATOM | 3209 | CD | LYS | B | 55 | 8.517 | 3.142 | 45.125 | 1.00 | 90.32 | C |
| ATOM | 3210 | CE | LYS | B | 55 | 7.432 | 3.223 | 46.179 | 1.00 | 91.78 | C |
| ATOM | 3211 | NZ | LYS | B | 55 | 8.001 | 3.130 | 47.545 | 1.00 | 90.99 | N |
| ATOM | 3212 | C | LYS | B | 55 | 11.604 | 4.576 | 46.510 | 1.00 | 64.35 | C |
| ATOM | 3213 | O | LYS | B | 55 | 11.052 | 5.402 | 47.261 | 1.00 | 64.87 | O |
| ATOM | 3214 | N | GLY | B | 56 | 12.102 | 3.411 | 46.921 | 1.00 | 58.81 | N |
| ATOM | 3215 | CA | GLY | B | 56 | 12.187 | 3.015 | 48.337 | 1.00 | 62.90 | C |
| ATOM | 3216 | C | GLY | B | 56 | 13.242 | 3.798 | 49.110 | 1.00 | 64.41 | C |
| ATOM | 3217 | O | GLY | B | 56 | 13.212 | 3.745 | 50.357 | 1.00 | 69.18 | O |
| ATOM | 3218 | N | ILE | B | 57 | 14.176 | 4.465 | 48.429 | 1.00 | 60.63 | N |
| ATOM | 3219 | CA | ILE | B | 57 | 15.252 | 5.275 | 49.086 | 1.00 | 59.43 | C |
| ATOM | 3220 | CB | ILE | B | 57 | 16.594 | 5.096 | 48.352 | 1.00 | 57.96 | C |
| ATOM | 3221 | CG1 | ILE | B | 57 | 17.084 | 3.646 | 48.450 | 1.00 | 57.59 | C |
| ATOM | 3222 | CG2 | ILE | B | 57 | 17.634 | 6.092 | 48.859 | 1.00 | 54.34 | C |
| ATOM | 3223 | CD1 | ILE | B | 57 | 18.231 | 3.302 | 47.524 | 1.00 | 57.21 | C |
| ATOM | 3224 | C | ILE | B | 57 | 14.827 | 6.752 | 49.171 | 1.00 | 60.43 | C |
| ATOM | 3225 | O | ILE | B | 57 | 15.198 | 7.415 | 50.182 | 1.00 | 61.38 | O |
| ATOM | 3226 | N | CYS | B | 58 | 14.083 | 7.250 | 48.170 | 1.00 | 62.92 | N |
| ATOM | 3227 | CA | CYS | B | 58 | 13.585 | 8.654 | 48.093 | 1.00 | 61.23 | C |
| ATOM | 3228 | CB | CYS | B | 58 | 14.695 | 9.614 | 47.690 | 1.00 | 58.81 | C |
| ATOM | 3229 | SG | CYS | B | 58 | 14.385 | 11.301 | 48.283 | 1.00 | 63.89 | S |
| ATOM | 3230 | C | CYS | B | 58 | 12.406 | 8.772 | 47.118 | 1.00 | 62.46 | C |
| ATOM | 3231 | O | CYS | B | 58 | 12.469 | 8.183 | 46.036 | 1.00 | 75.61 | O |
| ATOM | 3232 | N | GLU | B | 59 | 11.407 | 9.578 | 47.470 | 1.00 | 68.19 | N |
| ATOM | 3233 | CA | GLU | B | 59 | 10.048 | 9.572 | 46.863 | 1.00 | 79.40 | C |
| ATOM | 3234 | CB | GLU | B | 59 | 9.090 | 10.446 | 47.684 | 1.00 | 81.36 | C |
| ATOM | 3235 | CG | GLU | B | 59 | 7.632 | 10.057 | 47.538 | 1.00 | 86.82 | C |
| ATOM | 3236 | CD | GLU | B | 59 | 7.308 | 8.675 | 48.085 | 1.00 | 98.68 | C |
| ATOM | 3237 | OE1 | GLU | B | 59 | 7.176 | 8.551 | 49.324 | 1.00 | 93.49 | O |
| ATOM | 3238 | OE2 | GLU | B | 59 | 7.211 | 7.720 | 47.275 | 1.00 | 97.97 | O |
| ATOM | 3239 | C | GLU | B | 59 | 10.068 | 10.018 | 45.386 | 1.00 | 84.41 | C |
| ATOM | 3240 | O | GLU | B | 59 | 9.081 | 9.689 | 44.681 | 1.00 | 92.78 | O |
| ATOM | 3241 | N | ASP | B | 60 | 11.095 | 10.728 | 44.895 | 1.00 | 75.82 | N |
| ATOM | 3242 | CA | ASP | B | 60 | 11.060 | 11.235 | 43.490 | 1.00 | 77.06 | C |
| ATOM | 3243 | CB | ASP | B | 60 | 10.704 | 12.720 | 43.436 | 1.00 | 84.43 | C |
| ATOM | 3244 | CG | ASP | B | 60 | 10.501 | 13.225 | 42.021 | 1.00 | 88.23 | C |
| ATOM | 3245 | OD1 | ASP | B | 60 | 10.496 | 12.389 | 41.103 | 1.00 | 94.02 | O |
| ATOM | 3246 | OD2 | ASP | B | 60 | 10.358 | 14.444 | 41.849 | 1.00 | 93.06 | O |
| ATOM | 3247 | C | ASP | B | 60 | 12.371 | 10.947 | 42.760 | 1.00 | 68.03 | C |
| ATOM | 3248 | O | ASP | B | 60 | 13.109 | 11.865 | 42.396 | 1.00 | 61.30 | O |
| ATOM | 3249 | N | PRO | B | 61 | 12.640 | 9.662 | 42.434 | 1.00 | 61.79 | N |
| ATOM | 3250 | CA | PRO | B | 61 | 13.933 | 9.252 | 41.888 | 1.00 | 60.49 | C |
| ATOM | 3251 | CB | PRO | B | 61 | 13.840 | 7.721 | 41.842 | 1.00 | 57.16 | C |
| ATOM | 3252 | CG | PRO | B | 61 | 12.356 | 7.449 | 41.717 | 1.00 | 56.94 | C |
| ATOM | 3253 | CD | PRO | B | 61 | 11.695 | 8.537 | 42.536 | 1.00 | 60.56 | C |
| ATOM | 3254 | C | PRO | B | 61 | 14.193 | 9.813 | 40.483 | 1.00 | 57.79 | C |
| ATOM | 3255 | O | PRO | B | 61 | 13.292 | 9.825 | 39.667 | 1.00 | 55.97 | O |
| ATOM | 3256 | N | ARG | B | 62 | 15.427 | 10.255 | 40.254 | 1.00 | 51.83 | N |
| ATOM | 3257 | CA | ARG | B | 62 | 15.908 | 10.817 | 38.973 | 1.00 | 52.34 | C |
| ATOM | 3258 | CB | ARG | B | 62 | 15.913 | 12.347 | 39.053 | 1.00 | 59.51 | C |
| ATOM | 3259 | CG | ARG | B | 62 | 14.547 | 13.006 | 38.939 | 1.00 | 62.47 | C |
| ATOM | 3260 | CD | ARG | B | 62 | 13.905 | 12.780 | 37.579 | 1.00 | 71.28 | C |
| ATOM | 3261 | NE | ARG | B | 62 | 12.727 | 13.615 | 37.409 | 1.00 | 75.51 | N |
| ATOM | 3262 | CZ | ARG | B | 62 | 11.560 | 13.426 | 38.021 | 1.00 | 80.70 | C |
| ATOM | 3263 | NH1 | ARG | B | 62 | 11.384 | 12.404 | 38.844 | 1.00 | 79.47 | N |
| ATOM | 3264 | NH2 | ARG | B | 62 | 10.563 | 14.269 | 37.806 | 1.00 | 82.03 | N |
| ATOM | 3265 | C | ARG | B | 62 | 17.320 | 10.292 | 38.692 | 1.00 | 53.24 | C |
| ATOM | 3266 | O | ARG | B | 62 | 18.075 | 10.067 | 39.657 | 1.00 | 57.86 | O |
| ATOM | 3267 | N | LEU | B | 63 | 17.661 | 10.096 | 37.418 | 1.00 | 48.72 | N |
| ATOM | 3268 | CA | LEU | B | 63 | 19.055 | 9.884 | 36.965 | 1.00 | 48.17 | C |
| ATOM | 3269 | CB | LEU | B | 63 | 19.075 | 9.581 | 35.464 | 1.00 | 52.28 | C |
| ATOM | 3270 | CG | LEU | B | 63 | 19.005 | 8.110 | 35.071 | 1.00 | 56.10 | C |
| ATOM | 3271 | CD1 | LEU | B | 63 | 19.201 | 7.962 | 33.567 | 1.00 | 57.46 | C |
| ATOM | 3272 | CD2 | LEU | B | 63 | 20.043 | 7.294 | 35.824 | 1.00 | 56.13 | C |
| ATOM | 3273 | C | LEU | B | 63 | 19.853 | 11.156 | 37.238 | 1.00 | 48.66 | C |
| ATOM | 3274 | O | LEU | B | 63 | 20.952 | 11.061 | 37.800 | 1.00 | 50.57 | O |
| ATOM | 3275 | N | PHE | B | 64 | 19.339 | 12.292 | 36.775 | 1.00 | 44.35 | N |
| ATOM | 3276 | CA | PHE | B | 64 | 19.953 | 13.627 | 36.959 | 1.00 | 45.87 | C |
| ATOM | 3277 | CB | PHE | B | 64 | 20.390 | 14.219 | 35.612 | 1.00 | 47.67 | C |
| ATOM | 3278 | CG | PHE | B | 64 | 21.179 | 13.285 | 34.723 | 1.00 | 50.92 | C |
| ATOM | 3279 | CD1 | PHE | B | 64 | 22.518 | 13.016 | 34.976 | 1.00 | 52.42 | C |
| ATOM | 3280 | CE1 | PHE | B | 64 | 23.237 | 12.145 | 34.168 | 1.00 | 54.78 | C |
| ATOM | 3281 | CZ | PHE | B | 64 | 22.644 | 11.549 | 33.080 | 1.00 | 51.06 | C |
| ATOM | 3282 | CD2 | PHE | B | 64 | 20.590 | 12.664 | 33.632 | 1.00 | 52.16 | C |
| ATOM | 3283 | CE2 | PHE | m | 64 | 21.319 | 11.810 | 32.812 | 1.00 | 51.51 | C |
| ATOM | 3284 | C | PHE | B | 64 | 18.898 | 14.496 | 37.633 | 1.00 | 46.27 | C |
| ATOM | 3285 | O | PHE | B | 64 | 17.729 | 14.426 | 37.182 | 1.00 | 42.35 | O |
| ATOM | 3286 | N | VAL | B | 65 | 19.260 | 15.226 | 38.688 | 1.00 | 44.70 | N |
| ATOM | 3287 | CA | VAL | B | 65 | 18.401 | 16.333 | 39.196 | 1.00 | 55.15 | C |
| ATOM | 3288 | CB | VAL | B | 65 | 18.005 | 16.184 | 40.684 | 1.00 | 59.56 | C |
| ATOM | 3289 | CG1 | VAL | B | 65 | 17.608 | 14.751 | 41.039 | 1.00 | 55.61 | C |
| ATOM | 3290 | CG2 | VAL | B | 65 | 19.066 | 16.697 | 41.642 | 1.00 | 60.80 | C |
| ATOM | 3291 | C | VAL | B | 65 | 19.175 | 17.612 | 38.889 | 1.00 | 57.62 | C |
| ATOM | 3292 | O | VAL | B | 65 | 20.342 | 17.664 | 39.268 | 1.00 | 56.48 | O |
| ATOM | 3293 | N | ASP | B | 66 | 18.577 | 18.536 | 38.128 | 1.00 | 68.98 | N |
| ATOM | 3294 | CA | ASP | B | 66 | 19.241 | 19.758 | 37.585 | 1.00 | 78.73 | C |
| ATOM | 3295 | CB | ASP | B | 66 | 19.664 | 20.736 | 38.692 | 1.00 | 76.32 | C |
| ATOM | 3296 | CG | ASP | B | 66 | 18.704 | 20.873 | 39.862 | 1.00 | 77.04 | C |
| ATOM | 3297 | OD1 | ASP | B | 66 | 17.470 | 20.836 | 39.641 | 1.00 | 80.24 | O |
| ATOM | 3298 | OD2 | ASP | B | 66 | 19.202 | 21.020 | 40.992 | 1.00 | 71.38 | O |
| ATOM | 3299 | C | ASP | B | 66 | 20.490 | 19.387 | 36.756 | 1.00 | 91.31 | C |
| ATOM | 3300 | O | ASP | B | 66 | 21.477 | 20.173 | 36.799 | 1.00 | 96.40 | O |
| ATOM | 3301 | N | GLY | B | 67 | 20.482 | 18.255 | 36.036 | 1.00 | 87.87 | N |
| ATOM | 3302 | CA | GLY | B | 67 | 21.598 | 17.859 | 35.148 | 1.00 | 99.50 | C |
| ATOM | 3303 | C | GLY | B | 67 | 22.772 | 17.213 | 35.881 | 1.00 | 106.07 | C |
| ATOM | 3304 | O | GLY | B | 67 | 22.685 | 17.023 | 37.116 | 1.00 | 110.56 | O |
| ATOM | 3305 | N | ILE | B | 68 | 23.858 | 16.925 | 35.147 | 1.00 | 106.95 | N |
| ATOM | 3306 | CA | ILE | B | 68 | 24.808 | 15.801 | 35.438 | 1.00 | 101.00 | C |
| ATOM | 3307 | CB | ILE | B | 68 | 25.412 | 15.187 | 34.151 | 1.00 | 97.61 | C |
| ATOM | 3308 | CG1 | ILE | B | 68 | 26.400 | 14.069 | 34.497 | 1.00 | 100.01 | C |
| ATOM | 3309 | CG2 | ILE | B | 68 | 26.055 | 16.231 | 33.247 | 1.00 | 99.40 | C |
| ATOM | 3310 | CD1 | ILE | B | 68 | 26.342 | 12.863 | 33.592 | 1.00 | 100.63 | C |
| ATOM | 3311 | C | ILE | B | 68 | 25.883 | 16.210 | 36.460 | 1.00 | 98.23 | C |
| ATOM | 3312 | O | ILE | B | 68 | 25.968 | 15.516 | 37.495 | 1.00 | 103.20 | O |
| ATOM | 3313 | N | SER | B | 69 | 26.691 | 17.247 | 36.200 | 1.00 | 93.68 | N |
| ATOM | 3314 | CA | SER | B | 69 | 27.783 | 17.697 | 37.111 | 1.00 | 96.00 | C |
| ATOM | 3315 | CB | SER | B | 69 | 29.093 | 17.839 | 36.382 | 1.00 | 97.86 | C |
| ATOM | 3316 | OG | SER | B | 69 | 29.018 | 18.873 | 35.417 | 1.00 | 107.70 | O |
| ATOM | 3317 | C | SER | B | 69 | 27.374 | 18.995 | 37.825 | 1.00 | 97.06 | C |
| ATOM | 3318 | O | SER | B | 69 | 28.248 | 19.831 | 38.094 | 1.00 | 91.78 | O |
| ATOM | 3319 | N | SER | B | 70 | 26.082 | 19.137 | 38.135 | 1.00 | 103.04 | N |
| ATOM | 3320 | CA | SER | B | 70 | 25.507 | 20.215 | 38.983 | 1.00 | 98.74 | C |
| ATOM | 3321 | CB | SER | B | 70 | 24.067 | 20.456 | 38.604 | 1.00 | 98.44 | C |
| ATOM | 3322 | OG | SER | B | 70 | 23.402 | 19.220 | 38.381 | 1.00 | 94.83 | O |
| ATOM | 3323 | C | SER | B | 70 | 25.657 | 19.849 | 40.472 | 1.00 | 93.89 | C |
| ATOM | 3324 | O | SER | B | 70 | 25.887 | 20.770 | 41.296 | 1.00 | 70.41 | O |
| ATOM | 3325 | N | HIS | B | 71 | 25.537 | 18.558 | 40.816 | 1.00 | 86.16 | N |
| ATOM | 3326 | CA | HIS | B | 71 | 25.533 | 18.078 | 42.226 | 1.00 | 73.87 | C |
| ATOM | 3327 | CB | HIS | B | 71 | 24.310 | 17.218 | 42.502 | 1.00 | 68.15 | C |
| ATOM | 3328 | CG | HIS | B | 71 | 23.064 | 18.021 | 42.439 | 1.00 | 76.32 | C |
| ATOM | 3329 | ND1 | HIS | B | 71 | 22.780 | 19.014 | 43.356 | 1.00 | 80.06 | N |
| ATOM | 3330 | CE1 | HIS | B | 71 | 21.631 | 19.576 | 43.038 | 1.00 | 81.08 | C |
| ATOM | 3331 | NE2 | HIS | B | 71 | 21.167 | 18.982 | 41.935 | 1.00 | 72.92 | N |
| ATOM | 3332 | CD2 | HIS | B | 71 | 22.062 | 18.029 | 41.540 | 1.00 | 75.53 | C |
| ATOM | 3333 | C | HIS | B | 71 | 26.839 | 17.369 | 42.576 | 1.00 | 68.84 | C |
| ATOM | 3334 | O | HIS | B | 71 | 27.417 | 16.686 | 41.702 | 1.00 | 67.57 | O |
| ATOM | 3335 | N | ASP | B | 72 | 27.260 | 17.531 | 43.832 | 1.00 | 63.82 | N |
| ATOM | 3336 | CA | ASP | B | 72 | 28.544 | 16.999 | 44.351 | 1.00 | 57.92 | C |
| ATOM | 3337 | CB | ASP | B | 72 | 28.931 | 17.670 | 45.666 | 1.00 | 60.84 | C |
| ATOM | 3338 | CG | ASP | B | 72 | 28.598 | 19.148 | 45.707 | 1.00 | 58.00 | C |
| ATOM | 3339 | OD1 | ASP | B | 72 | 28.342 | 19.703 | 44.637 | 1.00 | 50.62 | O |
| ATOM | 3340 | OD2 | ASP | B | 72 | 28.595 | 19.720 | 46.819 | 1.00 | 58.71 | O |
| ATOM | 3341 | C | ASP | B | 72 | 28.433 | 15.473 | 44.473 | 1.00 | 51.87 | C |
| ATOM | 3342 | O | ASP | B | 72 | 27.287 | 14.931 | 44.570 | 1.00 | 41.29 | O |
| ATOM | 3343 | N | LEU | B | 73 | 29.584 | 14.812 | 44.382 | 1.00 | 42.39 | N |
| ATOM | 3344 | CA | LEU | B | 73 | 29.678 | 13.338 | 44.330 | 1.00 | 45.93 | C |
| ATOM | 3345 | CB | LEU | B | 73 | 30.378 | 12.847 | 43.054 | 1.00 | 41.42 | C |
| ATOM | 3346 | CG | LEU | B | 73 | 29.654 | 13.080 | 41.732 | 1.00 | 42.20 | C |
| ATOM | 3347 | CD1 | LEU | B | 73 | 30.474 | 12.486 | 40.609 | 1.00 | 44.67 | C |
| ATOM | 3348 | CD2 | LEU | B | 73 | 28.243 | 12.520 | 41.714 | 1.00 | 43.04 | C |
| ATOM | 3349 | C | LEU | B | 73 | 30.465 | 12.899 | 45.553 | 1.00 | 46.45 | C |
| ATOM | 3350 | O | LEU | B | 73 | 31.462 | 13.587 | 45.896 | 1.00 | 42.68 | O |
| ATOM | 3351 | N | HIS | B | 74 | 30.030 | 11.781 | 46.138 | 1.00 | 47.92 | N |
| ATOM | 3352 | CA | HIS | B | 74 | 30.653 | 11.144 | 47.321 | 1.00 | 53.30 | C |
| ATOM | 3353 | CB | HIS | B | 74 | 29.899 | 11.570 | 48.598 | 1.00 | 62.08 | C |
| ATOM | 3354 | CG | HIS | B | 74 | 29.829 | 13.056 | 48.778 | 1.00 | 68.91 | C |
| ATOM | 3355 | ND1 | HIS | B | 74 | 30.954 | 13.832 | 49.071 | 1.00 | 76.63 | N |
| ATOM | 3356 | CE1 | HIS | B | 74 | 30.604 | 15.104 | 49.145 | 1.00 | 69.30 | C |
| ATOM | 3357 | NE2 | HIS | B | 74 | 29.282 | 15.183 | 48.906 | 1.00 | 69.22 | N |
| ATOM | 3358 | CD2 | HIS | B | 74 | 28.792 | 13.921 | 48.677 | 1.00 | 68.74 | C |
| ATOM | 3359 | C | HIS | B | 74 | 30.744 | 9.633 | 47.066 | 1.00 | 48.73 | C |
| ATOM | 3360 | O | HIS | B | 74 | 29.751 | 9.028 | 46.607 | 1.00 | 46.48 | O |
| ATOM | 3361 | N | GLN | B | 75 | 31.946 | 9.083 | 47.229 | 1.00 | 45.08 | N |
| ATOM | 3362 | CA | GLN | B | 75 | 32.183 | 7.661 | 47.555 | 1.00 | 49.42 | C |
| ATOM | 3363 | CB | GLN | B | 75 | 33.679 | 7.356 | 47.470 | 1.00 | 46.29 | C |
| ATOM | 3364 | CG | GLN | B | 75 | 34.456 | 7.576 | 48.762 | 1.00 | 48.38 | C |
| ATOM | 3365 | CD | GLN | B | 75 | 34.745 | 9.015 | 49.130 | 1.00 | 49.87 | C |
| ATOM | 3366 | OE1 | GLN | B | 75 | 34.087 | 9.93 | 48.671 | 1.00 | 49.02 | O |
| ATOM | 3367 | NE2 | GLN | B | 75 | 35.745 | 9.223 | 49.977 | 1.00 | 51.33 | N |
| ATOM | 3368 | C | GLN | B | 75 | 31.602 | 7.504 | 48.957 | 1.00 | 57.52 | C |
| ATOM | 3369 | O | GLN | B | 75 | 31.847 | 8.403 | 49.784 | 1.00 | 78.07 | O |
| ATOM | 3370 | N | GLY | B | 76 | 30.780 | 6.503 | 49.217 | 1.00 | 58.59 | N |
| ATOM | 3371 | CA | GLY | B | 76 | 30.119 | 6.424 | 50.537 | 1.00 | 62.90 | C |
| ATOM | 3372 | C | GLY | B | 76 | 31.098 | 6.032 | 51.636 | 1.00 | 60.53 | C |
| ATOM | 3373 | O | GLY | B | 76 | 32.315 | 6.285 | 51.483 | 1.00 | 58.53 | O |
| ATOM | 3374 | N | GLN | B | 77 | 30.610 | 5.392 | 52.699 | 1.00 | 66.72 | N |
| ATOM | 3375 | CA | GLN | B | 77 | 31.481 | 4.885 | 53.793 | 1.00 | 65.19 | C |
| ATOM | 3376 | CB | GLN | B | 77 | 30.631 | 4.397 | 54.975 | 1.00 | 72.68 | C |
| ATOM | 3377 | CG | GLN | B | 77 | 31.208 | 4.771 | 56.347 | 1.00 | 82.42 | C |
| ATOM | 3378 | CD | GLN | B | 77 | 32.251 | 3.794 | 56.850 | 1.00 | 89.82 | C |
| ATOM | 3379 | OE1 | GLN | B | 77 | 31.986 | 2.600 | 56.996 | 1.00 | 99.53 | O |
| ATOM | 3380 | NE2 | GLN | B | 77 | 33.456 | 4.283 | 57.120 | 1.00 | 83.94 | N |
| ATOM | 3381 | C | GLN | B | 77 | 32.440 | 3.829 | 53.211 | 1.00 | 57.24 | C |
| ATOM | 3382 | O | GLN | B | 77 | 33.558 | 3.723 | 53.736 | 1.00 | 56.77 | O |
| ATOM | 3383 | N | VAL | B | 78 | 32.067 | 3.144 | 52.119 | 1.00 | 53.26 | N |
| ATOM | 3384 | CA | VAL | B | 78 | 32.871 | 2.027 | 51.523 | 1.00 | 53.93 | C |
| ATOM | 3385 | CB | VAL | B | 78 | 32.195 | 0.672 | 51.826 | 1.00 | 50.27 | C |
| ATOM | 3386 | CG1 | VAL | B | 78 | 32.186 | 0.395 | 53.319 | 1.00 | 48.92 | C |
| ATOM | 3387 | CG2 | VAL | B | 78 | 30.773 | 0.574 | 51.259 | 1.00 | 48.14 | C |
| ATOM | 3388 | C | VAL | B | 78 | 33.116 | 2.218 | 50.011 | 1.00 | 53.57 | C |
| ATOM | 3389 | O | VAL | B | 78 | 33.624 | 1.264 | 49.377 | 1.00 | 46.56 | O |
| ATOM | 3390 | N | GLY | B | 79 | 32.810 | 3.386 | 49.440 | 1.00 | 55.14 | N |
| ATOM | 3391 | CA | GLY | B | 79 | 33.002 | 3.651 | 47.998 | 1.00 | 55.83 | C |
| ATOM | 3392 | C | GLY | B | 79 | 34.458 | 3.949 | 47.684 | 1.00 | 50.78 | C |
| ATOM | 3393 | O | GLY | B | 79 | 35.235 | 4.072 | 48.623 | 1.00 | 48.48 | O |
| ATOM | 3394 | N | ASN | B | 80 | 34.808 | 4.094 | 46.403 | 1.00 | 53.93 | N |
| ATOM | 3395 | CA | ASN | B | 80 | 36.189 | 4.403 | 45.938 | 1.00 | 48.45 | C |
| ATOM | 3396 | CB | ASN | B | 80 | 36.633 | 3.376 | 44.903 | 1.00 | 50.77 | C |
| ATOM | 3397 | CG | ASN | B | 80 | 36.878 | 2.007 | 45.508 | 1.00 | 49.18 | C |
| ATOM | 3398 | OD1 | ASN | B | 80 | 37.973 | 1.724 | 46.001 | 1.00 | 45.07 | O |
| ATOM | 3399 | ND2 | ASN | B | 80 | 35.864 | 1.159 | 45.458 | 1.00 | 45.34 | N |
| ATOM | 3400 | C | ASN | B | 80 | 36.286 | 5.836 | 45.380 | 1.00 | 48.11 | C |
| ATOM | 3401 | O | ASN | B | 80 | 35.462 | 6.221 | 44.523 | 1.00 | 44.65 | O |
| ATOM | 3402 | N | CYS | B | 81 | 37.277 | 6.597 | 45.843 | 1.00 | 46.23 | N |
| ATOM | 3403 | CA | CYS | B | 81 | 37.537 | 8.004 | 45.445 | 1.00 | 46.19 | C |
| ATOM | 3404 | CB | CYS | B | 81 | 38.609 | 8.652 | 46.328 | 1.00 | 44.71 | C |
| ATOM | 3405 | SG | CYS | B | 81 | 37.979 | 9.244 | 47.933 | 1.00 | 50.70 | S |
| ATOM | 3406 | C | CYS | B | 81 | 37.892 | 8.019 | 43.947 | 1.00 | 49.28 | C |
| ATOM | 3407 | O | CYS | B | 81 | 37.375 | 8.912 | 43.221 | 1.00 | 50.74 | O |
| ATOM | 3408 | N | TRP | B | 82 | 38.689 | 7.047 | 43.483 | 1.00 | 47.39 | N |
| ATOM | 3409 | CA | TRP | B | 82 | 39.133 | 6.964 | 42.066 | 1.00 | 48.39 | C |
| ATOM | 3410 | CB | TRP | B | 82 | 40.131 | 5.818 | 41.818 | 1.00 | 49.38 | C |
| ATOM | 3411 | CG | TRP | B | 82 | 39.538 | 4.449 | 41.886 | 1.00 | 51.29 | C |
| ATOM | 3412 | CD1 | TRP | B | 82 | 39.693 | 3.549 | 42.897 | 1.00 | 52.07 | C |
| ATOM | 3413 | NE1 | TRP | B | 82 | 38.980 | 2.412 | 42.641 | 1.00 | 48.40 | N |
| ATOM | 3414 | CE2 | TRP | B | 82 | 38.345 | 2.545 | 41.436 | 1.00 | 53.08 | C |
| ATOM | 3415 | CD2 | TRP | B | 82 | 38.679 | 3.817 | 40.918 | 1.00 | 55.19 | C |
| ATOM | 3416 | CE3 | TRP | B | 82 | 38.156 | 4.194 | 39.678 | 1.00 | 53.36 | C |
| ATOM | 3417 | CZ3 | TRP | B | 82 | 37.332 | 3.319 | 39.005 | 1.00 | 56.76 | C |
| ATOM | 3418 | CH2 | TRP | B | 82 | 37.022 | 2.066 | 39.535 | 1.00 | 56.02 | C |
| ATOM | 3419 | CZ2 | TRP | B | 82 | 37.516 | 1.657 | 40.751 | 1.00 | 53.34 | C |
| ATOM | 3420 | C | TRP | B | 82 | 37.898 | 6.881 | 41.157 | 1.00 | 48.93 | C |
| ATOM | 3421 | O | TRP | B | 82 | 37.966 | 7.421 | 40.029 | 1.00 | 45.57 | O |
| ATOM | 3422 | N | PHE | B | 83 | 36.810 | 6.250 | 41.615 | 1.00 | 47.41 | N |
| ATOM | 3423 | CA | PHE | B | 83 | 35.577 | 6.077 | 40.805 | 1.00 | 47.47 | C |
| ATOM | 3424 | CB | PHE | B | 83 | 34.664 | 4.974 | 41.345 | 1.00 | 42.58 | C |
| ATOM | 3425 | CG | PHE | B | 83 | 33.449 | 4.755 | 40.482 | 1.00 | 38.30 | C |
| ATOM | 3426 | CD1 | PHE | B | 83 | 33.550 | 4.076 | 39.273 | 1.00 | 39.37 | C |
| ATOM | 3427 | CE1 | PHE | B | 83 | 32.453 | 3.929 | 38.441 | 1.00 | 37.44 | C |
| ATOM | 3428 | CZ | PHE | B | 83 | 31.241 | 4.461 | 38.808 | 1.00 | 37.70 | C |
| ATOM | 3429 | CD2 | PHE | B | 83 | 32.227 | 5.283 | 40.838 | 1.00 | 36.47 | C |
| ATOM | 3430 | CE2 | PHE | B | 83 | 31.127 | 5.135 | 40.003 | 1.00 | 40.78 | C |
| ATOM | 3431 | C | PHE | B | 83 | 34.827 | 7.411 | 40.736 | 1.00 | 51.30 | C |
| ATOM | 3432 | O | PHE | B | 83 | 34.195 | 7.683 | 39.692 | 1.00 | 55.79 | O |
| ATOM | 3433 | N | VAL | B | 84 | 34.871 | 8.203 | 41.814 | 1.00 | 49.09 | N |
| ATOM | 3434 | CA | VAL | B | 84 | 34.190 | 9.530 | 41.882 | 1.00 | 43.86 | C |
| ATOM | 3435 | CB | VAL | B | 84 | 34.211 | 10.104 | 43.308 | 1.00 | 47.92 | C |
| ATOM | 3436 | CG1 | VAL | B | 84 | 33.969 | 11.601 | 43.326 | 1.00 | 46.80 | C |
| ATOM | 3437 | CG2 | VAL | B | 84 | 33.202 | 9.396 | 44.196 | 1.00 | 48.47 | C |
| ATOM | 3438 | C | VAL | B | 84 | 34.877 | 10.455 | 40.880 | 1.00 | 38.56 | C |
| ATOM | 3439 | O | VAL | B | 84 | 34.176 | 11.079 | 40.095 | 1.00 | 39.35 | O |
| ATOM | 3440 | N | ALA | B | 85 | 36.204 | 10.459 | 40.885 | 1.00 | 35.03 | N |
| ATOM | 3441 | CA | ALA | B | 85 | 37.073 | 11.204 | 39.957 | 1.00 | 38.19 | C |
| ATOM | 3442 | CB | ALA | B | 85 | 38.514 | 10.887 | 40.245 | 1.00 | 39.33 | C |
| ATOM | 3443 | C | ALA | B | 85 | 36.727 | 10.866 | 38.502 | 1.00 | 41.90 | C |
| ATOM | 3444 | O | ALA | B | 85 | 36.553 | 11.807 | 37.714 | 1.00 | 45.51 | O |
| ATOM | 3445 | N | ALA | B | 86 | 36.633 | 9.578 | 38.162 | 1.00 | 45.30 | N |
| ATOM | 3446 | CA | ALA | B | 86 | 36.360 | 9.090 | 36.789 | 1.00 | 42.10 | C |
| ATOM | 3447 | CB | ALA | B | 86 | 36.559 | 7.597 | 36.703 | 1.00 | 41.20 | C |
| ATOM | 3448 | C | ALA | B | 86 | 34.943 | 9.502 | 36.364 | 1.00 | 41.75 | C |
| ATOM | 3449 | O | ALA | B | 86 | 34.767 | 9.863 | 35.201 | 1.00 | 40.95 | O |
| ATOM | 3450 | N | CYS | B | 87 | 33.953 | 9.440 | 37.252 | 1.00 | 43.66 | N |
| ATOM | 3451 | CA | CYS | B | 87 | 32.573 | 9.902 | 36.939 | 1.00 | 46.58 | C |
| ATOM | 3452 | CB | CYS | B | 87 | 31.590 | 9.498 | 38.027 | 1.00 | 45.23 | C |
| ATOM | 3453 | SG | CYS | B | 87 | 31.137 | 7.749 | 37.968 | 1.00 | 48.11 | S |
| ATOM | 3454 | C | CYS | B | 87 | 32.579 | 11.430 | 36.741 | 1.00 | 51.72 | C |
| ATOM | 3455 | O | CYS | B | 87 | 31.770 | 11.922 | 35.940 | 1.00 | 47.91 | O |
| ATOM | 3456 | N | SER | B | 88 | 33.466 | 12.151 | 37.439 | 1.00 | 48.85 | N |
| ATOM | 3457 | CA | SER | B | 88 | 33.600 | 13.625 | 37.345 | 1.00 | 50.49 | C |
| ATOM | 3458 | CB | SER | B | 88 | 34.534 | 14.154 | 38.387 | 1.00 | 48.82 | C |
| ATOM | 3459 | OG | SER | B | 88 | 34.468 | 15.559 | 38.440 | 1.00 | 46.81 | O |
| ATOM | 3460 | C | SER | B | 88 | 34.099 | 13.993 | 35.944 | 1.00 | 51.63 | C |
| ATOM | 3461 | O | SER | B | 88 | 33.488 | 14.868 | 35.323 | 1.00 | 50.14 | O |
| ATOM | 3462 | N | SER | B | 89 | 35.201 | 13.379 | 35.508 | 1.00 | 48.23 | N |
| ATOM | 3463 | CA | SER | B | 89 | 35.768 | 13.537 | 34.146 | 1.00 | 52.38 | C |
| ATOM | 3464 | CB | SER | B | 89 | 37.013 | 12.729 | 33.988 | 1.00 | 49.24 | C |
| ATOM | 3465 | OG | SER | B | 89 | 38.025 | 13.246 | 34.838 | 1.00 | 57.04 | O |
| ATOM | 3466 | C | SER | B | 89 | 34.691 | 13.167 | 33.117 | 1.00 | 55.07 | C |
| ATOM | 3467 | O | SER | B | 89 | 34.425 | 13.988 | 32.242 | 1.00 | 53.80 | O |
| ATOM | 3468 | N | LEU | B | 90 | 34.042 | 12.011 | 33.264 | 1.00 | 48.42 | N |
| ATOM | 3469 | CA | LEU | B | 90 | 33.006 | 11.550 | 32.312 | 1.00 | 50.12 | C |
| ATOM | 3470 | CB | LEU | B | 90 | 32.419 | 10.216 | 32.779 | 1.00 | 48.61 | C |
| ATOM | 3471 | CG | LEU | B | 90 | 31.348 | 9.629 | 31.862 | 1.00 | 46.33 | C |
| ATOM | 3472 | CD1 | LEU | B | 90 | 31.968 | 9.267 | 30.518 | 1.00 | 49.01 | C |
| ATOM | 3473 | CD2 | LEU | B | 90 | 30.666 | 8.431 | 32.504 | 1.00 | 46.90 | C |
| ATOM | 3474 | C | LEU | B | 90 | 31.918 | 12.621 | 32.196 | 1.00 | 51.48 | C |
| ATOM | 3475 | O | LEU | B | 90 | 31.452 | 12.908 | 31.059 | 1.00 | 53.21 | O |
| ATOM | 3476 | N | ALA | B | 91 | 31.496 | 13.175 | 33.327 | 1.00 | 52.24 | N |
| ATOM | 3477 | CA | ALA | B | 91 | 30.399 | 14.165 | 33.375 | 1.00 | 51.35 | C |
| ATOM | 3478 | CB | ALA | B | 91 | 29.943 | 14.378 | 34.793 | 1.00 | 54.24 | C |
| ATOM | 3479 | C | ALA | B | 91 | 30.845 | 15.478 | 32.718 | 1.00 | 43.72 | C |
| ATOM | 3480 | O | ALA | B | 91 | 29.953 | 16.226 | 32.351 | 1.00 | 49.28 | O |
| ATOM | 3481 | N | SER | B | 92 | 32.151 | 15.745 | 32.603 | 1.00 | 43.70 | N |
| ATOM | 3482 | CA | SER | B | 92 | 32.737 | 16.916 | 31.888 | 1.00 | 51.24 | C |
| ATOM | 3483 | CB | SER | B | 92 | 34.200 | 16.717 | 31.547 | 1.00 | 51.00 | C |
| ATOM | 3484 | OG | SER | B | 92 | 35.041 | 16.785 | 32.682 | 1.00 | 57.34 | O |
| ATOM | 3485 | C | SER | B | 92 | 31.993 | 17.163 | 30.568 | 1.00 | 64.84 | C |
| ATOM | 3486 | O | SER | B | 92 | 31.494 | 18.285 | 30.372 | 1.00 | 70.57 | O |
| ATOM | 3487 | N | ARG | B | 93 | 31.950 | 16.140 | 29.700 | 1.00 | 65.19 | N |
| ATOM | 3488 | CA | ARG | B | 93 | 31.703 | 16.259 | 28.239 | 1.00 | 61.10 | C |
| ATOM | 3489 | CB | ARG | B | 93 | 32.936 | 15.763 | 27.477 | 1.00 | 67.46 | C |
| ATOM | 3490 | CG | ARG | B | 93 | 34.272 | 16.316 | 27.963 | 1.00 | 72.77 | C |
| ATOM | 3491 | CD | ARG | B | 93 | 35.422 | 16.096 | 26.986 | 1.00 | 69.54 | C |
| ATOM | 3492 | NE | ARG | B | 93 | 35.502 | 17.221 | 26.075 | 1.00 | 78.20 | N |
| ATOM | 3493 | CZ | ARG | B | 93 | 34.710 | 17.425 | 25.018 | 1.00 | 84.62 | C |
| ATOM | 3494 | NH1 | ARG | B | 93 | 33.772 | 16.554 | 24.671 | 1.00 | 77.25 | N |
| ATOM | 3495 | NH2 | ARG | B | 93 | 34.877 | 18.514 | 24.286 | 1.00 | 91.79 | N |
| ATOM | 3496 | C | ARG | B | 93 | 30.481 | 15.436 | 27.810 | 1.00 | 53.24 | C |
| ATOM | 3497 | O | ARG | B | 93 | 30.598 | 14.204 | 27.781 | 1.00 | 59.94 | O |
| ATOM | 3498 | N | GLU | B | 94 | 29.401 | 16.099 | 27.384 | 1.00 | 55.26 | N |
| ATOM | 3499 | CA | GLU | B | 94 | 28.114 | 15.481 | 26.945 | 1.00 | 58.65 | C |
| ATOM | 3500 | CB | GLU | B | 94 | 27.175 | 16.504 | 26.301 | 1.00 | 64.16 | C |
| ATOM | 3501 | CG | GLU | B | 94 | 26.052 | 16.937 | 27.241 | 1.00 | 70.99 | C |
| ATOM | 3502 | CD | GLU | B | 94 | 24.766 | 17.423 | 26.588 | 1.00 | 71.48 | C |
| ATOM | 3503 | OE1 | GLU | B | 94 | 24.612 | 17.223 | 25.367 | 1.00 | 67.68 | O |
| ATOM | 3504 | OE2 | GLU | B | 94 | 23.914 | 17.991 | 27.312 | 1.00 | 72.19 | O |
| ATOM | 3505 | C | GLU | B | 94 | 28.311 | 14.301 | 25.988 | 1.00 | 55.63 | C |
| ATOM | 3506 | O | GLU | B | 94 | 27.556 | 13.337 | 26.115 | 1.00 | 54.95 | O |
| ATOM | 3507 | N | SER | B | 95 | 29.257 | 14.347 | 25.057 | 1.00 | 54.63 | N |
| ATOM | 3508 | CA | SER | B | 95 | 29.402 | 13.270 | 24.047 | 1.00 | 60.23 | C |
| ATOM | 3509 | CB | SER | B | 95 | 30.152 | 13.733 | 22.838 | 1.00 | 64.68 | C |
| ATOM | 3510 | OG | SER | B | 95 | 31.498 | 14.013 | 23.175 | 1.00 | 75.19 | O |
| ATOM | 3511 | C | SER | B | 95 | 30.074 | 12.038 | 24.662 | 1.00 | 62.19 | C |
| ATOM | 3512 | O | SER | B | 95 | 30.101 | 10.996 | 23.985 | 1.00 | 63.93 | O |
| ATOM | 3513 | N | LEU | B | 96 | 30.648 | 12.146 | 25.862 | 1.00 | 61.35 | N |
| ATOM | 3514 | CA | LEU | B | 96 | 31.334 | 10.994 | 26.499 | 1.00 | 55.97 | C |
| ATOM | 3515 | CB | LEU | B | 96 | 32.635 | 11.460 | 27.149 | 1.00 | 56.54 | C |
| ATOM | 3516 | CG | LEU | B | 96 | 33.699 | 11.938 | 26.159 | 1.00 | 57.47 | C |
| ATOM | 3517 | CD1 | LEU | B | 96 | 35.059 | 12.059 | 26.823 | 1.00 | 57.96 | C |
| ATOM | 3518 | CD2 | LEU | B | 96 | 33.804 | 10.999 | 24.966 | 1.00 | 61.54 | C |
| ATOM | 3519 | C | LEU | B | 96 | 30.364 | 10.321 | 27.469 | 1.00 | 54.90 | C |
| ATOM | 3520 | O | LEU | B | 96 | 30.178 | 9.083 | 27.346 | 1.00 | 55.80 | O |
| ATOM | 3521 | N | TRP | B | 97 | 29.677 | 11.096 | 28.308 | 1.00 | 49.91 | N |
| ATOM | 3522 | CA | TRP | B | 97 | 28.731 | 10.501 | 29.284 | 1.00 | 53.10 | C |
| ATOM | 3523 | CB | TRP | B | 97 | 28.403 | 11.438 | 30.456 | 1.00 | 54.98 | C |
| ATOM | 3524 | CG | TRP | B | 97 | 27.503 | 12.599 | 30.173 | 1.00 | 55.86 | C |
| ATOM | 3525 | CD1 | TRP | B | 97 | 27.886 | 13.901 | 30.027 | 1.00 | 54.62 | C |
| ATOM | 3526 | NE1 | TRP | B | 97 | 26.791 | 14.699 | 29.824 | 1.00 | 51.21 | N |
| ATOM | 3527 | CE2 | TRP | B | 97 | 25.661 | 13.930 | 29.866 | 1.00 | 49.91 | C |
| ATOM | 3528 | CD2 | TRP | B | 97 | 26.063 | 12.597 | 30.097 | 1.00 | 52.11 | C |
| ATOM | 3529 | CE3 | TRP | B | 97 | 25.082 | 11.606 | 30.168 | 1.00 | 50.98 | C |
| ATOM | 3530 | CZ3 | TRP | B | 97 | 23.761 | 11.964 | 30.032 | 1.00 | 48.57 | C |
| ATOM | 3531 | CH2 | TRP | B | 97 | 23.390 | 13.288 | 29.807 | 1.00 | 51.53 | C |
| ATOM | 3532 | CZ2 | TRP | B | 97 | 24.325 | 14.292 | 29.721 | 1.00 | 51.83 | C |
| ATOM | 3533 | C | TRP | B | 97 | 27.484 | 9.990 | 28.566 | 1.00 | 51.03 | C |
| ATOM | 3534 | O | TRP | B | 97 | 26.879 | 9.042 | 29.090 | 1.00 | 58.34 | O |
| ATOM | 3535 | N | GLN | B | 98 | 27.114 | 10.546 | 27.414 | 1.00 | 52.40 | N |
| ATOM | 3536 | CA | GLN | B | 98 | 25.978 | 9.992 | 26.621 | 1.00 | 52.46 | C |
| ATOM | 3537 | CB | GLN | B | 98 | 25.326 | 11.047 | 25.727 | 1.00 | 56.94 | C |
| ATOM | 3538 | CG | GLN | B | 98 | 24.620 | 12.163 | 26.490 | 1.00 | 58.46 | C |
| ATOM | 3539 | CD | GLN | B | 98 | 23.884 | 13.119 | 25.575 | 1.00 | 61.10 | C |
| ATOM | 3540 | OE1 | GLN | B | 98 | 24.264 | 13.352 | 24.424 | 1.00 | 56.24 | O |
| ATOM | 3541 | NE2 | GLN | B | 98 | 22.810 | 13.691 | 26.092 | 1.00 | 58.64 | N |
| ATOM | 3542 | C | GLN | B | 98 | 26.436 | 8.754 | 25.824 | 1.00 | 50.03 | C |
| ATOM | 3543 | O | GLN | B | 98 | 25.557 | 8.057 | 25.324 | 1.00 | 53.58 | O |
| ATOM | 3544 | N | LYS | B | 99 | 27.732 | 8.437 | 25.728 | 1.00 | 48.97 | N |
| ATOM | 3545 | CA | LYS | B | 99 | 28.160 | 7.105 | 25.212 | 1.00 | 54.44 | C |
| ATOM | 3546 | CB | LYS | B | 99 | 29.620 | 7.041 | 24.741 | 1.00 | 64.07 | C |
| ATOM | 3547 | CG | LYS | B | 99 | 29.937 | 7.774 | 23.442 | 1.00 | 75.27 | C |
| ATOM | 3548 | CD | LYS | B | 99 | 28.853 | 7.726 | 22.350 | 1.00 | 80.82 | C |
| ATOM | 3549 | CE | LYS | B | 99 | 29.160 | 8.595 | 21.141 | 1.00 | 83.08 | C |
| ATOM | 3550 | NZ | LYS | B | 99 | 29.135 | 10.050 | 21.455 | 1.00 | 82.04 | N |
| ATOM | 3551 | C | LYS | B | 99 | 27.987 | 6.074 | 26.322 | 1.00 | 51.14 | C |
| ATOM | 3552 | O | LYS | B | 99 | 27.483 | 4.982 | 26.039 | 1.00 | 53.41 | O |
| ATOM | 3553 | N | VAL | B | 100 | 28.421 | 6.390 | 27.539 | 1.00 | 49.67 | N |
| ATOM | 3554 | CA | VAL | B | 100 | 28.298 | 5.452 | 28.689 | 1.00 | 45.12 | C |
| ATOM | 3555 | CB | VAL | B | 100 | 29.208 | 5.870 | 29.851 | 1.00 | 38.60 | C |
| ATOM | 3556 | CG1 | VAL | B | 100 | 28.979 | 5.006 | 31.069 | 1.00 | 41.46 | C |
| ATOM | 3557 | CG2 | VAL | B | 100 | 30.669 | 5.826 | 29.433 | 1.00 | 39.86 | C |
| ATOM | 3558 | C | VAL | B | 100 | 26.816 | 5.325 | 29.082 | 1.00 | 45.97 | C |
| ATOM | 3559 | O | VAL | B | 100 | 26.422 | 4.210 | 29.447 | 1.00 | 43.32 | O |
| ATOM | 3560 | N | ILE | B | 101 | 26.015 | 6.386 | 28.939 | 1.00 | 45.31 | N |
| ATOM | 3561 | CA | ILE | B | 101 | 24.578 | 6.397 | 29.352 | 1.00 | 52.44 | C |
| ATOM | 3562 | CB | ILE | B | 101 | 24.303 | 7.434 | 30.461 | 1.00 | 53.07 | C |
| ATOM | 3563 | CG1 | ILE | B | 101 | 25.395 | 7.402 | 31.540 | 1.00 | 55.68 | C |
| ATOM | 3564 | CG2 | ILE | B | 101 | 22.915 | 7.207 | 31.036 | 1.00 | 47.25 | C |
| ATOM | 3565 | CD1 | ILE | B | 101 | 25.168 | 8.342 | 32.696 | 1.00 | 58.73 | C |
| ATOM | 3566 | C | ILE | B | 101 | 23.704 | 6.627 | 28.124 | 1.00 | 58.02 | C |
| ATOM | 3567 | O | ILE | B | 101 | 23.221 | 7.729 | 27.871 | 1.00 | 61.95 | O |
| ATOM | 3568 | N | PRO | B | 102 | 23.435 | 5.566 | 27.340 | 1.00 | 61.64 | N |
| ATOM | 3569 | CA | PRO | B | 102 | 22.700 | 5.721 | 26.085 | 1.00 | 60.32 | C |
| ATOM | 3570 | CB | PRO | B | 102 | 22.853 | 4.334 | 25.443 | 1.00 | 65.88 | C |
| ATOM | 3571 | CG | PRO | B | 102 | 23.020 | 3.390 | 26.616 | 1.00 | 64.13 | C |
| ATOM | 3572 | CD | PRO | B | 102 | 23.832 | 4.177 | 27.618 | 1.00 | 61.90 | C |
| ATOM | 3573 | C | PRO | B | 102 | 21.221 | 6.084 | 26.262 | 1.00 | 57.31 | C |
| ATOM | 3574 | O | PRO | B | 102 | 20.658 | 5.728 | 27.256 | 1.00 | 64.20 | O |
| ATOM | 3575 | N | ASP | B | 103 | 20.650 | 6.785 | 25.280 | 1.00 | 62.01 | N |
| ATOM | 3576 | CA | ASP | B | 103 | 19.208 | 7.144 | 25.179 | 1.00 | 64.52 | C |
| ATOM | 3577 | CB | ASP | B | 103 | 18.357 | 6.004 | 24.595 | 1.00 | 75.28 | C |
| ATOM | 3578 | CG | ASP | B | 103 | 19.014 | 5.145 | 23.517 | 1.00 | 82.90 | C |
| ATOM | 3579 | OD1 | ASP | B | 103 | 19.565 | 5.718 | 22.547 | 1.00 | 82.78 | O |
| ATOM | 3580 | OD2 | ASP | B | 103 | 18.966 | 3.891 | 23.652 | 1.00 | 86.34 | O |
| ATOM | 3581 | C | ASP | B | 103 | 18.732 | 7.569 | 26.571 | 1.00 | 62.60 | C |
| ATOM | 3582 | O | ASP | B | 103 | 17.750 | 7.010 | 27.078 | 1.00 | 65.77 | O |
| ATOM | 3583 | N | TRP | B | 104 | 19.404 | 8.550 | 27.165 | 1.00 | 68.52 | N |
| ATOM | 3584 | CA | TRP | B | 104 | 19.283 | 8.826 | 28.616 | 1.00 | 69.33 | C |
| ATOM | 3585 | CB | TRP | B | 104 | 20.355 | 9.805 | 29.110 | 1.00 | 73.38 | C |
| ATOM | 3586 | CG | TRP | B | 104 | 20.139 | 11.257 | 28.823 | 1.00 | 75.36 | C |
| ATOM | 3587 | CD1 | TRP | B | 104 | 20.748 | 12.003 | 27.855 | 1.00 | 76.19 | C |
| ATOM | 3588 | NE1 | TRP | B | 104 | 20.350 | 13.310 | 27.939 | 1.00 | 73.26 | N |
| ATOM | 3589 | CE2 | TRP | B | 104 | 19.472 | 13.441 | 28.982 | 1.00 | 81.05 | C |
| ATOM | 3590 | CD2 | TRP | B | 104 | 19.321 | 12.167 | 29.576 | 1.00 | 77.79 | C |
| ATOM | 3591 | CE3 | TRP | B | 104 | 18.475 | 12.031 | 30.681 | 1.00 | 74.28 | C |
| ATOM | 3592 | CZ3 | TRP | B | 104 | 17.816 | 13.146 | 31.152 | 1.00 | 79.19 | C |
| ATOM | 3593 | CH2 | TRP | B | 104 | 17.976 | 14.395 | 30.548 | 1.00 | 76.41 | C |
| ATOM | 3594 | CZ2 | TRP | B | 104 | 18.800 | 14.567 | 29.459 | 1.00 | 80.38 | C |
| ATOM | 3595 | C | TRP | B | 104 | 17.855 | 9.244 | 28.946 | 1.00 | 62.94 | C |
| ATOM | 3596 | O | TRP | B | 104 | 17.371 | 8.805 | 29.984 | 1.00 | 55.71 | O |
| ATOM | 3597 | N | LYS | B | 105 | 17.174 | 9.977 | 28.068 | 1.00 | 69.85 | N |
| ATOM | 3598 | CA | LYS | B | 105 | 15.785 | 10.446 | 28.342 | 1.00 | 73.61 | C |
| ATOM | 3599 | CB | LYS | B | 105 | 15.377 | 11.593 | 27.408 | 1.00 | 74.48 | C |
| ATOM | 3600 | CG | LYS | B | 105 | 16.042 | 12.934 | 27.699 | 1.00 | 79.69 | C |
| ATOM | 3601 | CD | LYS | B | 105 | 15.737 | 13.983 | 26.649 | 1.00 | 85.00 | C |
| ATOM | 3602 | CE | LYS | B | 105 | 16.850 | 14.989 | 26.425 | 1.00 | 84.35 | C |
| ATOM | 3603 | NZ | LYS | B | 105 | 16.620 | 15.793 | 25.200 | 1.00 | 81.96 | N |
| ATOM | 3604 | C | LYS | B | 105 | 14.809 | 9.261 | 28.263 | 1.00 | 69.52 | C |
| ATOM | 3605 | O | LYS | B | 105 | 13.735 | 9.350 | 28.884 | 1.00 | 72.46 | O |
| ATOM | 3606 | N | GLU | B | 106 | 15.160 | 8.186 | 27.556 | 1.00 | 70.98 | N |
| ATOM | 3607 | CA | GLU | B | 106 | 14.320 | 6.958 | 27.452 | 1.00 | 74.95 | C |
| ATOM | 3608 | CB | GLU | B | 106 | 14.710 | 6.196 | 26.180 | 1.00 | 88.35 | C |
| ATOM | 3609 | CG | GLU | B | 106 | 14.044 | 4.837 | 26.012 | 1.00 | 102.12 | C |
| ATOM | 3610 | CD | GLU | B | 106 | 12.625 | 4.827 | 25.461 | 1.00 | 109.45 | C |
| ATOM | 3611 | OE1 | GLU | B | 106 | 11.935 | 3.797 | 25.632 | 1.00 | 107.90 | O |
| ATOM | 3612 | OE2 | GLU | B | 106 | 12.216 | 5.837 | 24.846 | 1.00 | 122.74 | O |
| ATOM | 3613 | C | GLU | B | 106 | 14.443 | 6.125 | 28.746 | 1.00 | 70.46 | C |
| ATOM | 3614 | O | GLU | B | 106 | 13.493 | 5.367 | 29.047 | 1.00 | 63.39 | O |
| ATOM | 3615 | N | GLN | B | 107 | 15.555 | 6.260 | 29.483 | 1.00 | 61.88 | N |
| ATOM | 3616 | CA | GLN | B | 107 | 15.813 | 5.552 | 30.766 | 1.00 | 62.74 | C |
| ATOM | 3617 | CB | GLN | B | 107 | 17.308 | 5.283 | 30.945 | 1.00 | 60.23 | C |
| ATOM | 3618 | CG | GLN | B | 107 | 17.938 | 4.508 | 29.800 | 1.00 | 58.66 | C |
| ATOM | 3619 | CD | GLN | B | 107 | 19.062 | 3.613 | 30.263 | 1.00 | 57.31 | C |
| ATOM | 3620 | OE1 | GLN | B | 107 | 18.918 | 2.841 | 31.195 | 1.00 | 60.72 | O |
| ATOM | 3621 | NE2 | GLN | B | 107 | 20.200 | 3.679 | 29.602 | 1.00 | 60.22 | N |
| ATOM | 3622 | C | GLN | B | 107 | 15.294 | 6.367 | 31.964 | 1.00 | 62.69 | C |
| ATOM | 3623 | O | GLN | B | 107 | 14.838 | 5.742 | 32.941 | 1.00 | 67.47 | O |
| ATOM | 3624 | N | GLU | B | 108 | 15.403 | 7.698 | 31.911 | 1.00 | 63.27 | N |
| ATOM | 3625 | CA | GLU | B | 108 | 15.093 | 8.640 | 33.025 | 1.00 | 61.70 | C |
| ATOM | 3626 | CB | GLU | B | 108 | 15.340 | 10.091 | 32.580 | 1.00 | 64.41 | C |
| ATOM | 3627 | CG | GLU | B | 108 | 15.031 | 11.172 | 33.625 | 1.00 | 67.08 | C |
| ATOM | 3628 | CD | GLU | B | 108 | 15.883 | 11.135 | 34.888 | 1.00 | 65.25 | C |
| ATOM | 3629 | OE1 | GLU | B | 108 | 16.791 | 11.983 | 35.029 | 1.00 | 66.83 | O |
| ATOM | 3630 | OE2 | GLU | B | 108 | 15.651 | 10.245 | 35.720 | 1.00 | 59.77 | O |
| ATOM | 3631 | C | GLU | B | 108 | 13.645 | 8.407 | 33.467 | 1.00 | 61.11 | C |
| ATOM | 3632 | O | GLU | B | 108 | 12.804 | 8.142 | 32.588 | 1.00 | 61.41 | O |
| ATOM | 3633 | N | TRP | B | 109 | 13.374 | 8.466 | 34.778 | 1.00 | 57.01 | N |
| ATOM | 3634 | CA | TRP | B | 109 | 11.992 | 8.380 | 35.320 | 1.00 | 53.92 | C |
| ATOM | 3635 | CB | TRP | B | 109 | 11.974 | 8.431 | 36.859 | 1.00 | 49.57 | C |
| ATOM | 3636 | CG | TRP | B | 109 | 12.423 | 7.192 | 37.574 | 1.00 | 48.08 | C |
| ATOM | 3637 | CD1 | TRP | B | 109 | 11.626 | 6.191 | 38.055 | 1.00 | 46.58 | C |
| ATOM | 3638 | NE1 | TRP | B | 109 | 12.388 | 5.223 | 38.655 | 1.00 | 46.56 | N |
| ATOM | 3639 | CE2 | TRP | B | 109 | 13.705 | 5.591 | 38.608 | 1.00 | 44.54 | C |
| ATOM | 3640 | CD2 | TRP | B | 109 | 13.773 | 6.828 | 37.932 | 1.00 | 47.66 | C |
| ATOM | 3641 | CE3 | TRP | B | 109 | 15.029 | 7.414 | 37.742 | 1.00 | 46.27 | C |
| ATOM | 3642 | CZ3 | TRP | B | 109 | 16.148 | 6.771 | 38.226 | 1.00 | 46.60 | C |
| ATOM | 3643 | CH2 | TRP | B | 109 | 16.056 | 5.545 | 38.888 | 1.00 | 45.45 | C |
| ATOM | 3644 | CZ2 | TRP | B | 109 | 14.839 | 4.938 | 39.090 | 1.00 | 45.56 | C |
| ATOM | 3645 | C | TRP | B | 109 | 11.199 | 9.541 | 34.714 | 1.00 | 51.77 | C |
| ATOM | 3646 | O | TRP | B | 109 | 11.745 | 10.639 | 34.654 | 1.00 | 50.15 | O |
| ATOM | 3647 | N | ASP | B | 110 | 9.974 | 9.303 | 34.256 | 1.00 | 59.88 | N |
| ATOM | 3648 | CA | ASP | B | 110 | 9.012 | 10.381 | 33.914 | 1.00 | 61.43 | C |
| ATOM | 3649 | CB | ASP | B | 110 | 8.698 | 10.434 | 32.418 | 1.00 | 69.06 | C |
| ATOM | 3650 | CG | ASP | B | 110 | 7.751 | 11.561 | 32.027 | 1.00 | 71.48 | C |
| ATOM | 3651 | OD1 | ASP | B | 110 | 7.574 | 12.499 | 32.831 | 1.00 | 78.13 | O |
| ATOM | 3652 | OD2 | ASP | B | 110 | 7.193 | 11.489 | 30.923 | 1.00 | 73.72 | O |
| ATOM | 3653 | C | ASP | B | 110 | 7.741 | 10.154 | 34.718 | 1.00 | 65.42 | C |
| ATOM | 3654 | O | ASP | B | 110 | 7.046 | 9.160 | 34.501 | 1.00 | 69.33 | O |
| ATOM | 3655 | N | PRO | B | 111 | 7.405 | 11.068 | 35.654 | 1.00 | 70.31 | N |
| ATOM | 3656 | CA | PRO | B | 111 | 6.179 | 10.953 | 36.446 | 1.00 | 73.79 | C |
| ATOM | 3657 | CB | PRO | B | 111 | 6.254 | 12.136 | 37.428 | 1.00 | 80.92 | C |
| ATOM | 3658 | CG | PRO | B | 111 | 7.205 | 13.121 | 36.765 | 1.00 | 83.92 | C |
| ATOM | 3659 | CD | PRO | B | 111 | 8.197 | 12.257 | 36.014 | 1.00 | 76.92 | C |
| ATOM | 3660 | C | PRO | B | 111 | 4.887 | 11.026 | 35.617 | 1.00 | 78.58 | C |
| ATOM | 3661 | O | PRO | B | 111 | 3.897 | 10.481 | 36.068 | 1.00 | 87.95 | O |
| ATOM | 3662 | N | GLU | B | 112 | 4.920 | 11.658 | 34.438 | 1.00 | 79.99 | N |
| ATOM | 3663 | CA | GLU | B | 112 | 3.762 | 11.696 | 33.503 | 1.00 | 81.20 | C |
| ATOM | 3664 | CB | GLU | B | 112 | 3.930 | 12.809 | 32.469 | 1.00 | 83.50 | C |
| ATOM | 3665 | CG | GLU | B | 112 | 4.104 | 14.196 | 33.065 | 1.00 | 81.36 | C |
| ATOM | 3666 | CD | GLU | B | 112 | 3.810 | 15.311 | 32.077 | 1.00 | 82.11 | C |
| ATOM | 3667 | OE1 | GLU | B | 112 | 2.729 | 15.272 | 31.456 | 1.00 | 91.24 | O |
| ATOM | 3668 | OE2 | GLU | B | 112 | 4.661 | 16.198 | 31.911 | 1.00 | 73.35 | O |
| ATOM | 3669 | C | GLU | B | 112 | 3.600 | 10.330 | 32.813 | 1.00 | 83.30 | C |
| ATOM | 3670 | O | GLU | B | 112 | 2.494 | 10.049 | 32.316 | 1.00 | 86.77 | O |
| ATOM | 3671 | N | LYS | B | 113 | 4.651 | 9.507 | 32.780 | 1.00 | 79.15 | N |
| ATOM | 3672 | CA | LYS | B | 113 | 4.623 | 8.152 | 32.168 | 1.00 | 79.70 | C |
| ATOM | 3673 | CB | LYS | B | 113 | 5.573 | 8.090 | 30.968 | 1.00 | 82.30 | C |
| ATOM | 3674 | CG | LYS | B | 113 | 5.055 | 8.743 | 29.692 | 1.00 | 85.66 | C |
| ATOM | 3675 | CD | LYS | B | 113 | 6.145 | 9.094 | 28.683 | 1.00 | 88.75 | C |
| ATOM | 3676 | CE | LYS | B | 113 | 6.865 | 7.897 | 28.095 | 1.00 | 85.96 | C |
| ATOM | 3677 | NZ | LYS | B | 113 | 6.088 | 7.270 | 27.003 | 1.00 | 80.98 | N |
| ATOM | 3678 | C | LYS | B | 113 | 5.006 | 7.131 | 33.231 | 1.00 | 79.93 | C |
| ATOM | 3679 | O | LYS | B | 113 | 6.010 | 6.443 | 33.098 | 1.00 | 80.88 | O |
| ATOM | 3680 | N | PRO | B | 114 | 4.196 | 6.972 | 34.299 | 1.00 | 90.08 | N |
| ATOM | 3681 | CA | PRO | B | 114 | 4.606 | 6.188 | 35.468 | 1.00 | 87.66 | C |
| ATOM | 3682 | CB | PRO | B | 114 | 3.415 | 6.354 | 36.427 | 1.00 | 91.50 | C |
| ATOM | 3683 | CG | PRO | B | 114 | 2.232 | 6.633 | 35.519 | 1.00 | 89.68 | C |
| ATOM | 3684 | CD | PRO | B | 114 | 2.814 | 7.468 | 34.401 | 1.00 | 92.86 | C |
| ATOM | 3685 | C | PRO | B | 114 | 4.854 | 4.701 | 35.159 | 1.00 | 86.29 | C |
| ATOM | 3686 | O | PRO | B | 114 | 5.843 | 4.152 | 35.648 | 1.00 | 78.92 | O |
| ATOM | 3687 | N | ASN | B | 115 | 3.975 | 4.104 | 34.343 | 1.00 | 85.94 | N |
| ATOM | 3688 | CA | ASN | B | 115 | 3.982 | 2.663 | 33.965 | 1.00 | 86.28 | C |
| ATOM | 3689 | CB | ASN | B | 115 | 2.692 | 2.255 | 33.248 | 1.00 | 94.40 | C |
| ATOM | 3690 | CG | ASN | B | 115 | 1.528 | 2.095 | 34.205 | 1.00 | 104.04 | C |
| ATOM | 3691 | OD1 | ASN | B | 115 | 1.526 | 2.679 | 35.288 | 1.00 | 114.10 | O |
| ATOM | 3692 | ND2 | ASN | B | 115 | 0.540 | 1.299 | 33.825 | 1.00 | 103.71 | N |
| ATOM | 3693 | C | ASN | B | 115 | 5.210 | 2.329 | 33.112 | 1.00 | 81.97 | C |
| ATOM | 3694 | O | ASN | B | 115 | 5.527 | 1.130 | 33.003 | 1.00 | 88.73 | O |
| ATOM | 3695 | N | ALA | B | 116 | 5.888 | 3.337 | 32.550 | 1.00 | 79.72 | N |
| ATOM | 3696 | CA | ALA | B | 116 | 7.173 | 3.187 | 31.822 | 1.00 | 74.91 | C |
| ATOM | 3697 | CB | ALA | B | 116 | 7.650 | 4.528 | 31.322 | 1.00 | 71.84 | C |
| ATOM | 3698 | C | ALA | B | 116 | 8.234 | 2.523 | 32.716 | 1.00 | 70.16 | C |
| ATOM | 3699 | O | ALA | B | 116 | 9.096 | 1.829 | 32.176 | 1.00 | 70.55 | O |
| ATOM | 3700 | N | TYR | B | 117 | 8.206 | 2.744 | 34.031 | 1.00 | 70.54 | N |
| ATOM | 3701 | CA | TYR | B | 117 | 9.270 | 2.272 | 34.956 | 1.00 | 67.96 | C |
| ATOM | 3702 | CB | TYR | B | 117 | 9.082 | 2.828 | 36.369 | 1.00 | 67.94 | C |
| ATOM | 3703 | CG | TYR | B | 117 | 10.117 | 2.340 | 37.352 | 1.00 | 64.89 | C |
| ATOM | 3704 | CD1 | TYR | B | 117 | 11.471 | 2.537 | 37.118 | 1.00 | 60.75 | C |
| ATOM | 3705 | CE1 | TYR | B | 117 | 12.435 | 2.081 | 38.005 | 1.00 | 59.32 | C |
| ATOM | 3706 | CZ | TYR | B | 117 | 12.049 | 1.421 | 39.159 | 1.00 | 61.30 | C |
| ATOM | 3707 | OH | TYR | B | 117 | 12.989 | 0.981 | 40.050 | 1.00 | 52.66 | O |
| ATOM | 3708 | CE2 | TYR | B | 117 | 10.701 | 1.219 | 39.411 | 1.00 | 62.55 | C |
| ATOM | 3709 | CD2 | TYR | B | 117 | 9.750 | 1.671 | 38.508 | 1.00 | 61.55 | C |
| ATOM | 3710 | C | TYR | B | 117 | 9.276 | 0.741 | 34.992 | 1.00 | 62.49 | C |
| ATOM | 3711 | O | TYR | B | 117 | 8.211 | 0.131 | 35.114 | 1.00 | 69.52 | O |
| ATOM | 3712 | N | ALA | B | 118 | 10.454 | 0.139 | 34.886 | 1.00 | 56.37 | N |
| ATOM | 3713 | CA | ALA | B | 118 | 10.625 | −1.326 | 34.911 | 1.00 | 56.99 | C |
| ATOM | 3714 | CB | ALA | B | 118 | 10.678 | −1.814 | 33.486 | 1.00 | 58.39 | C |
| ATOM | 3715 | C | ALA | B | 118 | 11.868 | −1.729 | 35.719 | 1.00 | 56.23 | C |
| ATOM | 3716 | O | ALA | B | 118 | 12.222 | −2.914 | 35.698 | 1.00 | 64.16 | O |
| ATOM | 3717 | N | GLY | B | 119 | 12.487 | −0.802 | 36.448 | 1.00 | 58.90 | N |
| ATOM | 3718 | CA | GLY | B | 119 | 13.586 | −1.112 | 37.383 | 1.00 | 60.25 | C |
| ATOM | 3719 | C | GLY | B | 119 | 14.755 | −1.766 | 36.673 | 1.00 | 60.17 | C |
| ATOM | 3720 | O | GLY | B | 119 | 15.413 | −2.674 | 37.269 | 1.00 | 57.30 | O |
| ATOM | 3721 | N | ILE | B | 120 | 15.027 | −1.307 | 35.449 | 1.00 | 59.46 | N |
| ATOM | 3722 | CA | ILE | B | 120 | 16.173 | −1.801 | 34.636 | 1.00 | 58.17 | C |
| ATOM | 3723 | CB | ILE | B | 120 | 15.684 | −2.876 | 33.655 | 1.00 | 58.47 | C |
| ATOM | 3724 | CG1 | ILE | B | 120 | 16.779 | −3.297 | 32.675 | 1.00 | 59.20 | C |
| ATOM | 3725 | CG2 | ILE | B | 120 | 14.412 | −2.419 | 32.959 | 1.00 | 60.71 | C |
| ATOM | 3726 | CD1 | ILE | B | 120 | 16.522 | −4.636 | 32.015 | 1.00 | 64.63 | C |
| ATOM | 3727 | C | ILE | B | 120 | 16.868 | −0.621 | 33.948 | 1.00 | 54.91 | C |
| ATOM | 3728 | O | ILE | B | 120 | 16.186 | 0.344 | 33.574 | 1.00 | 50.31 | O |
| ATOM | 3729 | N | PHE | B | 121 | 18.193 | −0.715 | 33.830 | 1.00 | 53.37 | N |
| ATOM | 3730 | CA | PHE | B | 121 | 19.088 | 0.279 | 33.197 | 1.00 | 51.19 | C |
| ATOM | 3731 | CB | PHE | B | 121 | 19.743 | 1.158 | 34.268 | 1.00 | 51.68 | C |
| ATOM | 3732 | CG | PHE | B | 121 | 18.747 | 2.072 | 34.925 | 1.00 | 51.78 | C |
| ATOM | 3733 | CD1 | PHE | B | 121 | 18.464 | 3.316 | 34.385 | 1.00 | 48.61 | C |
| ATOM | 3734 | CE1 | PHE | B | 121 | 17.515 | 4.144 | 34.963 | 1.00 | 48.23 | C |
| ATOM | 3735 | CZ | PHE | B | 121 | 16.826 | 3.733 | 36.078 | 1.00 | 49.02 | C |
| ATOM | 3736 | CD2 | PHE | B | 121 | 18.030 | 1.657 | 36.035 | 1.00 | 51.99 | C |
| ATOM | 3737 | CE2 | PHE | B | 121 | 17.073 | 2.486 | 36.605 | 1.00 | 53.03 | C |
| ATOM | 3738 | C | PHE | B | 121 | 20.130 | −0.465 | 32.367 | 1.00 | 51.89 | C |
| ATOM | 3739 | O | PHE | B | 121 | 20.380 | −1.664 | 32.626 | 1.00 | 56.20 | O |
| ATOM | 3740 | N | HIS | B | 122 | 20.732 | 0.231 | 31.407 | 1.00 | 52.05 | N |
| ATOM | 3741 | CA | HIS | B | 122 | 21.852 | −0.303 | 30.594 | 1.00 | 51.48 | C |
| ATOM | 3742 | CB | HIS | B | 122 | 21.322 | −0.968 | 29.316 | 1.00 | 54.92 | C |
| ATOM | 3743 | CG | HIS | B | 122 | 20.891 | −0.026 | 28.246 | 1.00 | 53.42 | C |
| ATOM | 3744 | ND1 | HIS | B | 122 | 19.707 | 0.665 | 28.314 | 1.00 | 59.20 | N |
| ATOM | 3745 | CE1 | HIS | B | 122 | 19.559 | 1.402 | 27.230 | 1.00 | 59.43 | C |
| ATOM | 3746 | NE2 | HIS | B | 122 | 20.614 | 1.195 | 26.450 | 1.00 | 58.04 | N |
| ATOM | 3747 | CD2 | HIS | B | 122 | 21.451 | 0.301 | 27.065 | 1.00 | 52.03 | C |
| ATOM | 3748 | C | HIS | B | 122 | 22.856 | 0.813 | 30.329 | 1.00 | 47.64 | C |
| ATOM | 3749 | O | HIS | B | 122 | 22.456 | 1.964 | 30.217 | 1.00 | 48.87 | O |
| ATOM | 3750 | N | PHE | B | 123 | 24.120 | 0.444 | 30.221 | 1.00 | 46.03 | N |
| ATOM | 3751 | CA | PHE | B | 123 | 25.266 | 1.356 | 30.045 | 1.00 | 47.68 | C |
| ATOM | 3752 | CB | PHE | B | 123 | 25.894 | 1.624 | 31.415 | 1.00 | 47.24 | C |
| ATOM | 3753 | CG | PHE | B | 123 | 24.921 | 2.149 | 32.441 | 1.00 | 48.94 | C |
| ATOM | 3754 | CD1 | PHE | B | 123 | 24.120 | 1.293 | 33.171 | 1.00 | 49.10 | C |
| ATOM | 3755 | CE1 | PHE | B | 123 | 23.211 | 1.782 | 34.092 | 1.00 | 50.22 | C |
| ATOM | 3756 | CZ | PHE | B | 123 | 23.105 | 3.130 | 34.309 | 1.00 | 48.78 | C |
| ATOM | 3757 | CD2 | PHE | B | 123 | 24.800 | 3.505 | 32.673 | 1.00 | 48.97 | C |
| ATOM | 3758 | CE2 | PHE | B | 123 | 23.894 | 3.989 | 33.599 | 1.00 | 48.62 | C |
| ATOM | 3759 | C | PHE | B | 123 | 26.219 | 0.710 | 29.031 | 1.00 | 52.13 | C |
| ATOM | 3760 | O | PHE | B | 123 | 26.173 | −0.532 | 28.875 | 1.00 | 53.77 | O |
| ATOM | 3761 | N | HIS | B | 124 | 27.040 | 1.514 | 28.352 | 1.00 | 48.47 | N |
| ATOM | 3762 | CA | HIS | B | 124 | 28.034 | 1.027 | 27.367 | 1.00 | 50.23 | C |
| ATOM | 3763 | CB | HIS | B | 124 | 27.879 | 1.731 | 26.010 | 1.00 | 49.56 | C |
| ATOM | 3764 | CG | HIS | B | 124 | 26.592 | 1.460 | 25.309 | 1.00 | 54.24 | C |
| ATOM | 3765 | ND1 | HIS | B | 124 | 25.911 | 0.270 | 25.440 | 1.00 | 60.12 | N |
| ATOM | 3766 | CE1 | HIS | B | 124 | 24.814 | 0.306 | 24.703 | 1.00 | 56.76 | C |
| ATOM | 3767 | NE2 | HIS | B | 124 | 24.765 | 1.485 | 24.092 | 1.00 | 56.00 | N |
| ATOM | 3768 | CD2 | HIS | B | 124 | 25.869 | 2.212 | 24.449 | 1.00 | 57.44 | C |
| ATOM | 3769 | C | HIS | B | 124 | 29.422 | 1.242 | 27.959 | 1.00 | 47.71 | C |
| ATOM | 3770 | O | HIS | B | 124 | 29.682 | 2.360 | 28.398 | 1.00 | 52.27 | O |
| ATOM | 3771 | N | PHE | B | 125 | 30.269 | 0.214 | 27.953 | 1.00 | 47.78 | N |
| ATOM | 3772 | CA | PHE | B | 125 | 31.697 | 0.295 | 28.356 | 1.00 | 48.71 | C |
| ATOM | 3773 | CB | PHE | B | 125 | 31.938 | −0.422 | 29.692 | 1.00 | 49.47 | C |
| ATOM | 3774 | CG | PHE | B | 125 | 31.109 | 0.078 | 30.854 | 1.00 | 50.46 | C |
| ATOM | 3775 | CD1 | PHE | B | 125 | 31.484 | 1.200 | 31.569 | 1.00 | 54.67 | C |
| ATOM | 3776 | CE1 | PHE | B | 125 | 30.726 | 1.637 | 32.645 | 1.00 | 60.57 | C |
| ATOM | 3777 | CZ | PHE | B | 125 | 29.577 | 0.978 | 33.005 | 1.00 | 57.93 | C |
| ATOM | 3778 | CD2 | PHE | B | 125 | 29.953 | −0.584 | 31.243 | 1.00 | 56.10 | C |
| ATOM | 3779 | CE2 | PHE | B | 125 | 29.193 | −0.142 | 32.313 | 1.00 | 54.77 | C |
| ATOM | 3780 | C | PHE | B | 125 | 32.549 | −0.283 | 27.221 | 1.00 | 48.85 | C |
| ATOM | 3781 | O | PHE | B | 125 | 32.019 | −1.000 | 26.354 | 1.00 | 50.08 | O |
| ATOM | 3782 | N | TRP | B | 126 | 33.834 | 0.031 | 27.231 | 1.00 | 43.42 | N |
| ATOM | 3783 | CA | TRP | B | 126 | 34.776 | −0.343 | 26.161 | 1.00 | 46.55 | C |
| ATOM | 3784 | CB | TRP | B | 126 | 35.563 | 0.873 | 25.666 | 1.00 | 48.27 | C |
| ATOM | 3785 | CG | TRP | B | 126 | 36.592 | 0.584 | 24.611 | 1.00 | 51.33 | C |
| ATOM | 3786 | CD1 | TRP | B | 126 | 37.938 | 0.423 | 24.786 | 1.00 | 53.97 | C |
| ATOM | 3787 | NE1 | TRP | B | 126 | 38.563 | 0.238 | 23.578 | 1.00 | 51.96 | N |
| ATOM | 3788 | CE2 | TRP | B | 126 | 37.625 | 0.281 | 22.584 | 1.00 | 47.84 | C |
| ATOM | 3789 | CD2 | TRP | B | 126 | 36.370 | 0.486 | 23.192 | 1.00 | 47.12 | C |
| ATOM | 3790 | CE3 | TRP | B | 126 | 35.236 | 0.569 | 22.377 | 1.00 | 54.83 | C |
| ATOM | 3791 | CZ3 | TRP | B | 126 | 35.376 | 0.428 | 21.011 | 1.00 | 50.65 | C |
| ATOM | 3792 | CH2 | TRP | B | 126 | 36.629 | 0.223 | 20.436 | 1.00 | 49.41 | C |
| ATOM | 3793 | CZ2 | TRP | B | 126 | 37.770 | 0.146 | 21.204 | 1.00 | 48.75 | C |
| ATOM | 3794 | C | TRP | B | 126 | 35.695 | −1.410 | 26.730 | 1.00 | 46.29 | C |
| ATOM | 3795 | O | TRP | B | 126 | 36.572 | −1.070 | 27.541 | 1.00 | 43.89 | O |
| ATOM | 3796 | N | ARG | B | 127 | 35.484 | −2.650 | 26.307 | 1.00 | 51.02 | N |
| ATOM | 3797 | CA | ARG | B | 127 | 36.303 | −3.808 | 26.734 | 1.00 | 53.74 | C |
| ATOM | 3798 | CB | ARG | B | 127 | 35.461 | −4.698 | 27.652 | 1.00 | 57.13 | C |
| ATOM | 3799 | CG | ARG | B | 127 | 34.878 | −3.965 | 28.858 | 1.00 | 66.79 | C |
| ATOM | 3800 | CD | ARG | B | 127 | 35.904 | −3.450 | 29.864 | 1.00 | 66.07 | C |
| ATOM | 3801 | NE | ARG | B | 127 | 36.610 | −4.492 | 30.608 | 1.00 | 60.27 | N |
| ATOM | 3802 | CZ | ARG | B | 127 | 37.864 | −4.396 | 31.051 | 1.00 | 66.67 | C |
| ATOM | 3803 | NH1 | ARG | B | 127 | 38.584 | −3.309 | 30.821 | 1.00 | 66.23 | N |
| ATOM | 3804 | NH2 | ARG | B | 127 | 38.405 | −5.404 | 31.717 | 1.00 | 76.09 | N |
| ATOM | 3805 | C | ARG | B | 127 | 36.810 | −4.530 | 25.483 | 1.00 | 49.77 | C |
| ATOM | 3806 | O | ARG | B | 127 | 35.980 | −4.863 | 24.612 | 1.00 | 48.55 | O |
| ATOM | 3807 | N | PHE | B | 128 | 38.127 | −4.706 | 25.402 | 1.00 | 48.26 | N |
| ATOM | 3808 | CA | PHE | B | 128 | 38.838 | −5.497 | 24.365 | 1.00 | 56.22 | C |
| ATOM | 3809 | CB | PHE | B | 128 | 38.681 | −6.984 | 24.679 | 1.00 | 58.00 | C |
| ATOM | 3810 | CG | PHE | B | 128 | 39.160 | −7.319 | 26.066 | 1.00 | 56.98 | C |
| ATOM | 3811 | CD1 | PHE | B | 128 | 40.516 | −7.330 | 26.353 | 1.00 | 57.43 | C |
| ATOM | 3812 | CE1 | PHE | B | 128 | 40.966 | −7.588 | 27.637 | 1.00 | 61.93 | C |
| ATOM | 3813 | CZ | PHE | B | 128 | 40.060 | −7.836 | 28.647 | 1.00 | 61.12 | C |
| ATOM | 3814 | CD2 | PHE | B | 128 | 38.258 | −7.539 | 27.098 | 1.00 | 60.95 | C |
| ATOM | 3815 | CE2 | PHE | B | 128 | 38.708 | −7.810 | 28.383 | 1.00 | 59.37 | C |
| ATOM | 3816 | C | PHE | B | 128 | 38.353 | −5.079 | 22.972 | 1.00 | 62.67 | C |
| ATOM | 3817 | O | PHE | B | 128 | 37.835 | −5.933 | 22.234 | 1.00 | 63.93 | O |
| ATOM | 3818 | N | GLY | B | 129 | 38.509 | −3.787 | 22.651 | 1.00 | 64.12 | N |
| ATOM | 3819 | CA | GLY | B | 129 | 38.339 | −3.228 | 21.299 | 1.00 | 61.25 | C |
| ATOM | 3820 | C | GLY | B | 129 | 36.888 | −3.170 | 20.849 | 1.00 | 60.88 | C |
| ATOM | 3821 | O | GLY | B | 129 | 36.688 | −3.042 | 19.619 | 1.00 | 67.21 | O |
| ATOM | 3822 | N | GLU | B | 130 | 35.922 | −3.194 | 21.779 | 1.00 | 57.01 | N |
| ATOM | 3823 | CA | GLU | B | 130 | 34.465 | −3.288 | 21.471 | 1.00 | 62.06 | C |
| ATOM | 3824 | CB | GLU | B | 130 | 34.098 | −4.771 | 21.386 | 1.00 | 75.44 | C |
| ATOM | 3825 | CG | GLU | B | 130 | 32.713 | −5.052 | 20.809 | 1.00 | 85.06 | C |
| ATOM | 3826 | CD | GLU | B | 130 | 32.235 | −6.493 | 20.952 | 1.00 | 82.11 | C |
| ATOM | 3827 | OE1 | GLU | B | 130 | 32.474 | −7.095 | 22.031 | 1.00 | 75.64 | O |
| ATOM | 3828 | OE2 | GLU | B | 130 | 31.628 | −7.008 | 19.986 | 1.00 | 85.90 | O |
| ATOM | 3829 | C | GLU | B | 130 | 33.596 | −2.577 | 22.532 | 1.00 | 56.89 | C |
| ATOM | 3830 | O | GLU | B | 130 | 33.910 | −2.650 | 23.719 | 1.00 | 53.92 | O |
| ATOM | 3831 | N | TRP | B | 131 | 32.482 | −1.975 | 22.119 | 1.00 | 58.14 | N |
| ATOM | 3832 | CA | TRP | B | 131 | 31.464 | −1.402 | 23.033 | 1.00 | 61.83 | C |
| ATOM | 3833 | CB | TRP | B | 131 | 30.660 | −0.261 | 22.386 | 1.00 | 64.05 | C |
| ATOM | 3834 | CG | TRP | B | 131 | 31.426 | 1.027 | 22.310 | 1.00 | 69.67 | C |
| ATOM | 3835 | CD1 | TRP | B | 131 | 32.033 | 1.566 | 21.212 | 1.00 | 71.32 | C |
| ATOM | 3836 | NE1 | TRP | B | 131 | 32.670 | 2.731 | 21.542 | 1.00 | 71.05 | N |
| ATOM | 3837 | CE2 | TRP | B | 131 | 32.492 | 2.981 | 22.872 | 1.00 | 72.12 | C |
| ATOM | 3838 | CD2 | TRP | B | 131 | 31.714 | 1.925 | 23.396 | 1.00 | 74.04 | C |
| ATOM | 3839 | CE3 | TRP | B | 131 | 31.383 | 1.947 | 24.754 | 1.00 | 72.70 | C |
| ATOM | 3840 | CZ3 | TRP | B | 131 | 31.824 | 2.995 | 25.529 | 1.00 | 68.03 | C |
| ATOM | 3841 | CH2 | TRP | B | 131 | 32.585 | 4.029 | 24.984 | 1.00 | 69.09 | C |
| ATOM | 3842 | CZ2 | TRP | B | 131 | 32.936 | 4.043 | 23.655 | 1.00 | 69.81 | C |
| ATOM | 3843 | C | TRP | B | 131 | 30.571 | −2.536 | 23.548 | 1.00 | 63.04 | C |
| ATOM | 3844 | O | TRP | B | 131 | 29.842 | −3.158 | 22.761 | 1.00 | 62.07 | O |
| ATOM | 3845 | N | VAL | B | 132 | 30.654 | −2.779 | 24.852 | 1.00 | 65.58 | N |
| ATOM | 3846 | CA | VAL | B | 132 | 29.898 | −3.826 | 25.590 | 1.00 | 60.32 | C |
| ATOM | 3847 | CB | VAL | B | 132 | 30.826 | −4.461 | 26.647 | 1.00 | 68.44 | C |
| ATOM | 3848 | CG1 | VAL | B | 132 | 30.057 | −5.108 | 27.783 | 1.00 | 74.86 | C |
| ATOM | 3849 | CG2 | VAL | B | 132 | 31.810 | −5.448 | 26.028 | 1.00 | 67.21 | C |
| ATOM | 3850 | C | VAL | B | 132 | 28.681 | −3.127 | 26.198 | 1.00 | 52.49 | C |
| ATOM | 3851 | O | VAL | B | 132 | 28.836 | −1.980 | 26.632 | 1.00 | 53.34 | O |
| ATOM | 3852 | N | ASP | B | 133 | 27.525 | −3.786 | 26.212 | 1.00 | 48.36 | N |
| ATOM | 3853 | CA | ASP | B | 133 | 26.257 | −3.264 | 26.787 | 1.00 | 53.80 | C |
| ATOM | 3854 | CB | ASP | B | 133 | 25.120 | −3.413 | 25.776 | 1.00 | 53.95 | C |
| ATOM | 3855 | CG | ASP | B | 133 | 23.751 | −3.056 | 26.313 | 1.00 | 64.31 | C |
| ATOM | 3856 | OD1 | ASP | B | 133 | 23.460 | −1.854 | 26.397 | 1.00 | 75.00 | O |
| ATOM | 3857 | OD2 | ASP | B | 133 | 22.994 | −3.986 | 26.659 | 1.00 | 81.48 | O |
| ATOM | 3858 | C | ASP | B | 133 | 25.960 | −4.012 | 28.101 | 1.00 | 55.57 | C |
| ATOM | 3859 | O | ASP | B | 133 | 25.591 | −5.192 | 28.017 | 1.00 | 53.95 | O |
| ATOM | 3860 | N | VAL | B | 134 | 26.114 | −3.359 | 29.264 | 1.00 | 50.51 | N |
| ATOM | 3861 | CA | VAL | B | 134 | 25.866 | −3.979 | 30.605 | 1.00 | 46.96 | C |
| ATOM | 3862 | CB | VAL | B | 134 | 26.930 | −3.573 | 31.627 | 1.00 | 47.97 | C |
| ATOM | 3863 | CG1 | VAL | B | 134 | 26.614 | −4.189 | 32.980 | 1.00 | 51.46 | C |
| ATOM | 3864 | CG2 | VAL | B | 134 | 28.321 | −3.967 | 31.159 | 1.00 | 50.28 | C |
| ATOM | 3865 | C | VAL | B | 134 | 24.470 | −3.620 | 31.125 | 1.00 | 46.12 | C |
| ATOM | 3866 | O | VAL | B | 134 | 24.239 | −2.446 | 31.442 | 1.00 | 51.21 | O |
| ATOM | 3867 | N | VAL | B | 135 | 23.588 | −4.613 | 31.236 | 1.00 | 46.22 | N |
| ATOM | 3868 | CA | VAL | B | 135 | 22.213 | −4.478 | 31.799 | 1.00 | 49.63 | C |
| ATOM | 3869 | CB | VAL | B | 135 | 21.249 | −5.484 | 31.152 | 1.00 | 49.86 | C |
| ATOM | 3870 | CG1 | VAL | B | 135 | 19.872 | −5.446 | 31.798 | 1.00 | 52.31 | C |
| ATOM | 3871 | CG2 | VAL | B | 135 | 21.150 | −5.262 | 29.659 | 1.00 | 52.53 | C |
| ATOM | 3872 | C | VAL | B | 135 | 22.284 | −4.716 | 33.310 | 1.00 | 50.45 | C |
| ATOM | 3873 | O | VAL | B | 135 | 23.093 | −5.565 | 33.726 | 1.00 | 52.18 | O |
| ATOM | 3874 | N | ILE | B | 136 | 21.454 | −4.002 | 34.083 | 1.00 | 50.25 | N |
| ATOM | 3875 | CA | ILE | B | 136 | 21.342 | −4.135 | 35.568 | 1.00 | 44.24 | C |
| ATOM | 3876 | CB | ILE | B | 136 | 22.343 | −3.226 | 36.302 | 1.00 | 48.21 | C |
| ATOM | 3877 | CG1 | ILE | B | 136 | 22.091 | −1.747 | 35.998 | 1.00 | 54.00 | C |
| ATOM | 3878 | CG2 | ILE | B | 136 | 23.783 | −3.625 | 36.016 | 1.00 | 50.99 | C |
| ATOM | 3879 | CD1 | ILE | B | 136 | 22.833 | −0.802 | 36.916 | 1.00 | 55.16 | C |
| ATOM | 3880 | C | ILE | B | 136 | 19.912 | −3.815 | 35.993 | 1.00 | 43.21 | C |
| ATOM | 3881 | O | ILE | B | 136 | 19.215 | −3.093 | 35.253 | 1.00 | 40.72 | O |
| ATOM | 3882 | N | ASP | B | 137 | 19.496 | −4.332 | 37.158 | 1.00 | 47.60 | N |
| ATOM | 3883 | CA | ASP | B | 137 | 18.285 | −3.846 | 37.876 | 1.00 | 46.91 | C |
| ATOM | 3884 | CB | ASP | B | 137 | 17.621 | −4.985 | 38.669 | 1.00 | 51.66 | C |
| ATOM | 3885 | CG | ASP | B | 137 | 18.351 | −5.363 | 39.960 | 1.00 | 56.29 | C |
| ATOM | 3886 | OD1 | ASP | B | 137 | 18.284 | −4.568 | 40.911 | 1.00 | 47.22 | O |
| ATOM | 3887 | OD2 | ASP | B | 137 | 19.021 | −6.425 | 39.995 | 1.00 | 60.53 | O |
| ATOM | 3888 | C | ASP | B | 137 | 18.756 | −2.648 | 38.711 | 1.00 | 42.79 | C |
| ATOM | 3889 | O | ASP | B | 137 | 19.971 | −2.558 | 38.977 | 1.00 | 40.45 | O |
| ATOM | 3890 | N | ASP | B | 138 | 17.856 | −1.785 | 39.163 | 1.00 | 47.24 | N |
| ATOM | 3891 | CA | ASP | B | 138 | 18.253 | −0.548 | 39.892 | 1.00 | 52.69 | C |
| ATOM | 3892 | CB | ASP | B | 138 | 17.387 | 0.623 | 39.440 | 1.00 | 50.98 | C |
| ATOM | 3893 | CG | ASP | B | 138 | 15.923 | 0.401 | 39.744 | 1.00 | 50.28 | C |
| ATOM | 3894 | OD1 | ASP | B | 138 | 15.615 | −0.709 | 40.200 | 1.00 | 51.51 | O |
| ATOM | 3895 | OD2 | ASP | B | 138 | 15.115 | 1.337 | 39.531 | 1.00 | 49.49 | O |
| ATOM | 3896 | C | ASP | B | 138 | 18.179 | −0.717 | 41.425 | 1.00 | 58.68 | C |
| ATOM | 3897 | O | ASP | B | 138 | 17.955 | 0.317 | 42.112 | 1.00 | 56.46 | O |
| ATOM | 3898 | N | ARG | B | 139 | 18.356 | −1.927 | 41.978 | 1.00 | 54.57 | N |
| ATOM | 3899 | CA | ARG | B | 139 | 18.429 | −2.091 | 43.458 | 1.00 | 48.49 | C |
| ATOM | 3900 | CB | ARG | B | 139 | 18.140 | −3.501 | 43.975 | 1.00 | 51.23 | C |
| ATOM | 3901 | CG | ARG | B | 139 | 16.756 | −4.072 | 43.695 | 1.00 | 56.28 | C |
| ATOM | 3902 | CD | ARG | B | 139 | 17.003 | −5.571 | 43.781 | 1.00 | 60.44 | C |
| ATOM | 3903 | NE | ARG | B | 139 | 15.875 | −6.439 | 43.490 | 1.00 | 68.35 | N |
| ATOM | 3904 | CZ | ARG | B | 139 | 15.981 | −7.684 | 43.034 | 1.00 | 65.66 | C |
| ATOM | 3905 | NH1 | ARG | B | 139 | 17.168 | −8.217 | 42.774 | 1.00 | 68.43 | N |
| ATOM | 3906 | NH2 | ARG | B | 139 | 14.885 | −8.384 | 42.813 | 1.00 | 62.87 | N |
| ATOM | 3907 | C | ARG | B | 139 | 19.855 | −1.772 | 43.891 | 1.00 | 46.53 | C |
| ATOM | 3908 | O | ARG | B | 139 | 20.779 | −2.225 | 43.207 | 1.00 | 43.54 | O |
| ATOM | 3909 | N | LEU | B | 140 | 20.022 | −1.038 | 44.994 | 1.00 | 47.04 | N |
| ATOM | 3910 | CA | LEU | B | 140 | 21.358 | −0.587 | 45.453 | 1.00 | 45.08 | C |
| ATOM | 3911 | CB | LEU | B | 140 | 21.468 | 0.918 | 45.168 | 1.00 | 46.68 | C |
| ATOM | 3912 | CG | LEU | B | 140 | 21.564 | 1.300 | 43.685 | 1.00 | 43.15 | C |
| ATOM | 3913 | CD1 | LEU | B | 140 | 20.840 | 2.603 | 43.410 | 1.00 | 45.32 | C |
| ATOM | 3914 | CD2 | LEU | B | 140 | 23.011 | 1.396 | 43.233 | 1.00 | 42.28 | C |
| ATOM | 3915 | C | LEU | B | 140 | 21.570 | −0.921 | 46.931 | 1.00 | 44.13 | C |
| ATOM | 3916 | O | LEU | B | 140 | 20.618 | −0.905 | 47.726 | 1.00 | 37.77 | O |
| ATOM | 3917 | N | PRO | B | 141 | 22.850 | −1.183 | 47.309 | 1.00 | 46.01 | N |
| ATOM | 3918 | CA | PRO | B | 141 | 23.277 | −1.412 | 48.690 | 1.00 | 50.15 | C |
| ATOM | 3919 | CB | PRO | B | 141 | 24.823 | −1.472 | 48.624 | 1.00 | 48.28 | C |
| ATOM | 3920 | CG | PRO | B | 141 | 25.120 | −1.800 | 47.192 | 1.00 | 46.80 | C |
| ATOM | 3921 | CD | PRO | B | 141 | 24.009 | −1.139 | 46.409 | 1.00 | 47.14 | C |
| ATOM | 3922 | C | PRO | B | 141 | 22.854 | −0.219 | 49.558 | 1.00 | 54.23 | C |
| ATOM | 3923 | O | PRO | B | 141 | 23.062 | 0.894 | 49.129 | 1.00 | 63.28 | O |
| ATOM | 3924 | N | THR | B | 142 | 22.251 | −0.471 | 50.718 | 1.00 | 59.05 | N |
| ATOM | 3925 | CA | THR | B | 142 | 21.689 | 0.563 | 51.627 | 1.00 | 57.07 | C |
| ATOM | 3926 | CB | THR | B | 142 | 20.177 | 0.686 | 51.420 | 1.00 | 60.17 | C |
| ATOM | 3927 | OG1 | THR | B | 142 | 19.957 | 1.947 | 50.799 | 1.00 | 63.92 | O |
| ATOM | 3928 | CG2 | THR | B | 142 | 19.356 | 0.570 | 52.686 | 1.00 | 57.32 | C |
| ATOM | 3929 | C | THR | B | 142 | 22.014 | 0.166 | 53.058 | 1.00 | 56.15 | C |
| ATOM | 3930 | O | THR | B | 142 | 21.816 | −1.020 | 53.379 | 1.00 | 57.26 | O |
| ATOM | 3931 | N | VAL | B | 143 | 22.466 | 1.111 | 53.878 | 1.00 | 57.32 | N |
| ATOM | 3932 | CA | VAL | B | 143 | 22.646 | 0.874 | 55.337 | 1.00 | 56.71 | C |
| ATOM | 3933 | CB | VAL | B | 143 | 24.065 | 1.208 | 55.801 | 1.00 | 55.66 | C |
| ATOM | 3934 | CG1 | VAL | B | 143 | 24.151 | 1.414 | 57.299 | 1.00 | 57.57 | C |
| ATOM | 3935 | CG2 | VAL | B | 143 | 25.013 | 0.108 | 55.367 | 1.00 | 59.11 | C |
| ATOM | 3936 | C | VAL | B | 143 | 21.542 | 1.602 | 56.102 | 1.00 | 61.56 | C |
| ATOM | 3937 | O | VAL | B | 143 | 20.659 | 0.904 | 56.605 | 1.00 | 77.04 | O |
| ATOM | 3938 | N | ASN | B | 144 | 21.550 | 2.926 | 56.187 | 1.00 | 65.67 | N |
| ATOM | 3939 | CA | ASN | B | 144 | 20.520 | 3.625 | 57.001 | 1.00 | 69.11 | C |
| ATOM | 3940 | CB | ASN | B | 144 | 21.113 | 4.659 | 57.956 | 1.00 | 75.64 | C |
| ATOM | 3941 | CG | ASN | B | 144 | 21.582 | 4.052 | 59.252 | 1.00 | 71.47 | C |
| ATOM | 3942 | OD1 | ASN | B | 144 | 20.848 | 3.288 | 59.865 | 1.00 | 74.09 | O |
| ATOM | 3943 | ND2 | ASN | B | 144 | 22.783 | 4.408 | 59.676 | 1.00 | 75.22 | N |
| ATOM | 3944 | C | ASN | B | 144 | 19.566 | 4.336 | 56.065 | 1.00 | 64.60 | C |
| ATOM | 3945 | O | ASN | B | 144 | 19.480 | 5.552 | 56.199 | 1.00 | 69.91 | O |
| ATOM | 3946 | N | ASN | B | 145 | 18.921 | 3.604 | 55.152 | 1.00 | 67.46 | N |
| ATOM | 3947 | CA | ASN | B | 145 | 18.164 | 4.184 | 54.006 | 1.00 | 64.18 | C |
| ATOM | 3948 | CB | ASN | B | 145 | 16.893 | 4.884 | 54.491 | 1.00 | 66.40 | C |
| ATOM | 3949 | CG | ASN | B | 145 | 15.825 | 4.997 | 53.423 | 1.00 | 73.40 | C |
| ATOM | 3950 | OD1 | ASN | B | 145 | 15.745 | 4.158 | 52.529 | 1.00 | 81.09 | O |
| ATOM | 3951 | ND2 | ASN | B | 145 | 14.996 | 6.027 | 53.511 | 1.00 | 69.76 | N |
| ATOM | 3952 | C | ASN | B | 145 | 19.074 | 5.148 | 53.217 | 1.00 | 58.65 | C |
| ATOM | 3953 | O | ASN | B | 145 | 18.541 | 6.116 | 52.645 | 1.00 | 58.63 | O |
| ATOM | 3954 | N | GLN | B | 146 | 20.385 | 4.866 | 53.175 | 1.00 | 55.26 | N |
| ATOM | 3955 | CA | GLN | B | 146 | 21.452 | 5.705 | 52.565 | 1.00 | 56.25 | C |
| ATOM | 3956 | CB | GLN | B | 146 | 22.289 | 6.400 | 53.629 | 1.00 | 54.21 | C |
| ATOM | 3957 | CG | GLN | B | 146 | 21.649 | 7.651 | 54.190 | 1.00 | 56.33 | C |
| ATOM | 3958 | CD | GLN | B | 146 | 22.495 | 8.127 | 55.343 | 1.00 | 61.28 | C |
| ATOM | 3959 | OE1 | GLN | B | 146 | 23.713 | 8.290 | 55.220 | 1.00 | 61.99 | O |
| ATOM | 3960 | NE2 | GLN | B | 146 | 21.863 | 8.284 | 56.495 | 1.00 | 60.24 | N |
| ATOM | 3961 | C | GLN | B | 146 | 22.439 | 4.852 | 51.763 | 1.00 | 56.26 | C |
| ATOM | 3962 | O | GLN | B | 146 | 22.935 | 3.840 | 52.315 | 1.00 | 59.33 | O |
| ATOM | 3963 | N | LEU | B | 147 | 22.761 | 5.289 | 50.541 | 1.00 | 50.70 | N |
| ATOM | 3964 | CA | LEU | B | 147 | 23.731 | 4.610 | 49.638 | 1.00 | 46.40 | C |
| ATOM | 3965 | CB | LEU | B | 147 | 23.774 | 5.326 | 48.280 | 1.00 | 42.56 | C |
| ATOM | 3966 | CG | LEU | B | 147 | 22.459 | 5.267 | 47.500 | 1.00 | 40.85 | C |
| ATOM | 3967 | CD1 | LEU | B | 147 | 22.437 | 6.221 | 46.320 | 1.00 | 40.17 | C |
| ATOM | 3968 | CD2 | LEU | B | 147 | 22.191 | 3.853 | 47.033 | 1.00 | 46.52 | C |
| ATOM | 3969 | C | LEU | B | 147 | 25.088 | 4.611 | 50.335 | 1.00 | 43.40 | C |
| ATOM | 3970 | O | LEU | B | 147 | 25.433 | 5.627 | 50.948 | 1.00 | 49.50 | O |
| ATOM | 3971 | N | ILE | B | 148 | 25.798 | 3.496 | 50.277 | 1.00 | 40.55 | N |
| ATOM | 3972 | CA | ILE | B | 148 | 27.084 | 3.297 | 51.002 | 1.00 | 42.24 | C |
| ATOM | 3973 | CB | ILE | B | 148 | 27.036 | 1.986 | 51.799 | 1.00 | 46.55 | C |
| ATOM | 3974 | CG1 | ILE | B | 148 | 26.446 | 0.838 | 50.970 | 1.00 | 47.89 | C |
| ATOM | 3975 | CG2 | ILE | B | 148 | 26.263 | 2.211 | 53.085 | 1.00 | 47.14 | C |
| ATOM | 3976 | CD1 | ILE | B | 148 | 26.667 | −0.518 | 51.566 | 1.00 | 50.53 | C |
| ATOM | 3977 | C | ILE | B | 148 | 28.236 | 3.311 | 50.009 | 1.00 | 41.88 | C |
| ATOM | 3978 | O | ILE | B | 148 | 29.396 | 3.407 | 50.434 | 1.00 | 40.87 | O |
| ATOM | 3979 | N | TYR | B | 149 | 27.939 | 3.207 | 48.723 | 1.00 | 47.13 | N |
| ATOM | 3980 | CA | TYR | B | 149 | 28.947 | 3.384 | 47.648 | 1.00 | 48.64 | C |
| ATOM | 3981 | CB | TYR | B | 149 | 28.890 | 2.188 | 46.686 | 1.00 | 47.91 | C |
| ATOM | 3982 | CG | TYR | B | 149 | 29.131 | 0.837 | 47.324 | 1.00 | 45.32 | C |
| ATOM | 3983 | CD1 | TYR | B | 149 | 30.422 | 0.365 | 47.510 | 1.00 | 40.58 | C |
| ATOM | 3984 | CE1 | TYR | B | 149 | 30.665 | −0.870 | 48.084 | 1.00 | 42.23 | C |
| ATOM | 3985 | CZ | TYR | B | 149 | 29.603 | −1.669 | 48.477 | 1.00 | 46.29 | C |
| ATOM | 3986 | OH | TYR | B | 149 | 29.848 | −2.889 | 49.051 | 1.00 | 48.16 | O |
| ATOM | 3987 | CE2 | TYR | B | 149 | 28.301 | −1.224 | 48.299 | 1.00 | 44.58 | C |
| ATOM | 3988 | CD2 | TYR | B | 149 | 28.076 | 0.021 | 47.728 | 1.00 | 46.62 | C |
| ATOM | 3989 | C | TYR | B | 149 | 28.691 | 4.779 | 47.060 | 1.00 | 48.70 | C |
| ATOM | 3990 | O | TYR | B | 149 | 28.249 | 5.663 | 47.810 | 1.00 | 47.24 | O |
| ATOM | 3991 | N | CYS | B | 150 | 28.943 | 4.997 | 45.770 | 1.00 | 45.21 | N |
| ATOM | 3992 | CA | CYS | B | 150 | 28.946 | 6.351 | 45.187 | 1.00 | 41.67 | C |
| ATOM | 3993 | CB | CYS | B | 150 | 29.774 | 6.336 | 43.918 | 1.00 | 49.60 | C |
| ATOM | 3994 | SG | CYS | B | 150 | 31.470 | 5.853 | 44.325 | 1.00 | 62.59 | S |
| ATOM | 3995 | C | CYS | B | 150 | 27.513 | 6.870 | 45.039 | 1.00 | 39.12 | C |
| ATOM | 3996 | O | CYS | B | 150 | 26.610 | 6.084 | 44.751 | 1.00 | 38.42 | O |
| ATOM | 3997 | N | HIS | B | 151 | 27.311 | 8.151 | 45.334 | 1.00 | 36.98 | N |
| ATOM | 3998 | CA | HIS | B | 151 | 25.995 | 8.836 | 45.268 | 1.00 | 39.63 | C |
| ATOM | 3999 | CB | HIS | B | 151 | 25.172 | 8.543 | 46.533 | 1.00 | 47.03 | C |
| ATOM | 4000 | CG | HIS | B | 151 | 25.852 | 8.977 | 47.794 | 1.00 | 48.87 | C |
| ATOM | 4001 | ND1 | HIS | B | 151 | 26.862 | 8.232 | 48.390 | 1.00 | 48.42 | N |
| ATOM | 4002 | CE1 | HIS | B | 151 | 27.291 | 8.857 | 49.466 | 1.00 | 51.18 | C |
| ATOM | 4003 | NE2 | HIS | B | 151 | 26.616 | 10.008 | 49.578 | 1.00 | 51.90 | N |
| ATOM | 4004 | CD2 | HIS | B | 151 | 25.709 | 10.086 | 48.549 | 1.00 | 50.12 | C |
| ATOM | 4005 | C | HIS | B | 151 | 26.238 | 10.334 | 45.073 | 1.00 | 39.82 | C |
| ATOM | 4006 | O | HIS | B | 151 | 27.421 | 10.769 | 45.030 | 1.00 | 36.22 | O |
| ATOM | 4007 | N | SER | B | 152 | 25.170 | 11.108 | 45.017 | 1.00 | 40.25 | N |
| ATOM | 4008 | CA | SER | B | 152 | 25.266 | 12.579 | 44.900 | 1.00 | 48.76 | C |
| ATOM | 4009 | CB | SER | B | 152 | 24.482 | 13.052 | 43.731 | 1.00 | 53.32 | C |
| ATOM | 4010 | OG | SER | B | 152 | 24.489 | 14.462 | 43.714 | 1.00 | 65.49 | O |
| ATOM | 4011 | C | SER | B | 152 | 24.787 | 13.224 | 46.199 | 1.00 | 48.86 | C |
| ATOM | 4012 | O | SER | B | 152 | 24.247 | 12.503 | 47.020 | 1.00 | 52.41 | O |
| ATOM | 4013 | N | ASN | B | 153 | 24.994 | 14.531 | 46.350 | 1.00 | 51.69 | N |
| ATOM | 4014 | CA | ASN | B | 153 | 24.447 | 15.368 | 47.448 | 1.00 | 51.15 | C |
| ATOM | 4015 | CB | ASN | B | 153 | 24.853 | 16.834 | 47.333 | 1.00 | 64.62 | C |
| ATOM | 4016 | CG | ASN | B | 153 | 26.111 | 17.156 | 48.102 | 1.00 | 75.20 | C |
| ATOM | 4017 | OD1 | ASN | B | 153 | 26.845 | 16.255 | 48.506 | 1.00 | 81.46 | O |
| ATOM | 4018 | ND2 | ASN | B | 153 | 26.362 | 18.439 | 48.307 | 1.00 | 79.34 | N |
| ATOM | 4019 | C | ASN | B | 153 | 22.922 | 15.351 | 47.434 | 1.00 | 49.63 | C |
| ATOM | 4020 | O | ASN | B | 153 | 22.355 | 15.408 | 48.513 | 1.00 | 51.56 | O |
| ATOM | 4021 | N | SER | B | 154 | 22.273 | 15.333 | 46.270 | 1.00 | 49.96 | N |
| ATOM | 4022 | CA | SER | B | 154 | 20.800 | 15.155 | 46.211 | 1.00 | 57.81 | C |
| ATOM | 4023 | CB | SER | B | 154 | 20.145 | 15.761 | 44.992 | 1.00 | 57.52 | C |
| ATOM | 4024 | OG | SER | B | 154 | 20.978 | 15.643 | 43.867 | 1.00 | 73.76 | O |
| ATOM | 4025 | C | SER | B | 154 | 20.511 | 13.661 | 46.354 | 1.00 | 58.91 | C |
| ATOM | 4026 | O | SER | B | 154 | 21.012 | 12.874 | 45.538 | 1.00 | 59.34 | O |
| ATOM | 4027 | N | ARG | B | 155 | 19.765 | 13.289 | 47.397 | 1.00 | 62.63 | N |
| ATOM | 4028 | CA | ARG | B | 155 | 19.551 | 11.867 | 47.780 | 1.00 | 61.27 | C |
| ATOM | 4029 | CB | ARG | B | 155 | 19.129 | 11.758 | 49.252 | 1.00 | 60.96 | C |
| ATOM | 4030 | CG | ARG | B | 155 | 17.747 | 12.301 | 49.591 | 1.00 | 61.14 | C |
| ATOM | 4031 | CD | ARG | B | 155 | 17.499 | 11.990 | 51.057 | 1.00 | 66.15 | C |
| ATOM | 4032 | NE | ARG | B | 155 | 16.287 | 12.544 | 51.653 | 1.00 | 68.76 | N |
| ATOM | 4033 | CZ | ARG | B | 155 | 16.013 | 12.495 | 52.959 | 1.00 | 69.28 | C |
| ATOM | 4034 | NH1 | ARG | B | 155 | 16.866 | 11.916 | 53.791 | 1.00 | 72.90 | N |
| ATOM | 4035 | NH2 | ARG | B | 155 | 14.886 | 13.007 | 53.431 | 1.00 | 64.36 | N |
| ATOM | 4036 | C | ARG | B | 155 | 18.554 | 11.208 | 46.815 | 1.00 | 56.00 | C |
| ATOM | 4037 | O | ARG | B | 155 | 18.273 | 10.021 | 47.007 | 1.00 | 54.29 | O |
| ATOM | 4038 | N | ASN | B | 156 | 18.049 | 11.941 | 45.819 | 1.00 | 50.41 | N |
| ATOM | 4039 | CA | ASN | B | 156 | 17.153 | 11.385 | 44.776 | 1.00 | 49.96 | C |
| ATOM | 4040 | CB | ASN | B | 156 | 15.789 | 12.080 | 44.747 | 1.00 | 46.88 | C |
| ATOM | 4041 | CG | ASN | B | 156 | 15.857 | 13.506 | 44.239 | 1.00 | 50.69 | C |
| ATOM | 4042 | OD1 | ASN | B | 156 | 16.868 | 14.193 | 44.395 | 1.00 | 51.25 | O |
| ATOM | 4043 | ND2 | ASN | B | 156 | 14.775 | 13.965 | 43.637 | 1.00 | 52.47 | N |
| ATOM | 4044 | C | ASN | B | 156 | 17.846 | 11.430 | 43.404 | 1.00 | 47.05 | C |
| ATOM | 4045 | O | ASN | B | 156 | 17.084 | 11.272 | 42.418 | 1.00 | 41.85 | O |
| ATOM | 4046 | N | GLU | B | 157 | 19.190 | 11.582 | 43.351 | 1.00 | 39.98 | N |
| ATOM | 4047 | CA | GLU | B | 157 | 20.022 | 11.481 | 42.110 | 1.00 | 47.77 | C |
| ATOM | 4048 | CB | GLU | B | 157 | 21.003 | 12.661 | 41.978 | 1.00 | 47.98 | C |
| ATOM | 4049 | CG | GLU | B | 157 | 21.760 | 12.683 | 40.650 | 1.00 | 49.02 | C |
| ATOM | 4050 | CD | GLU | B | 157 | 22.578 | 13.922 | 40.282 | 1.00 | 53.33 | C |
| ATOM | 4051 | OE1 | GLU | B | 157 | 23.775 | 13.984 | 40.639 | 1.00 | 49.97 | O |
| ATOM | 4052 | OE2 | GLU | B | 157 | 22.030 | 14.811 | 39.588 | 1.00 | 62.88 | O |
| ATOM | 4053 | C | GLU | B | 157 | 20.769 | 10.132 | 42.086 | 1.00 | 47.92 | C |
| ATOM | 4054 | O | GLU | B | 157 | 21.539 | 9.859 | 43.037 | 1.00 | 50.13 | O |
| ATOM | 4055 | N | PHE | B | 158 | 20.629 | 9.342 | 41.009 | 1.00 | 46.06 | N |
| ATOM | 4056 | CA | PHE | B | 158 | 21.104 | 7.932 | 40.983 | 1.00 | 43.84 | C |
| ATOM | 4057 | CB | PHE | B | 158 | 19.894 | 7.005 | 40.915 | 1.00 | 43.51 | C |
| ATOM | 4058 | CG | PHE | B | 158 | 19.055 | 7.125 | 42.160 | 1.00 | 43.23 | C |
| ATOM | 4059 | CD1 | PHE | B | 158 | 19.402 | 6.433 | 43.308 | 1.00 | 40.92 | C |
| ATOM | 4060 | CE1 | PHE | B | 158 | 18.664 | 6.578 | 44.470 | 1.00 | 43.06 | C |
| ATOM | 4061 | CZ | PHE | B | 158 | 17.582 | 7.432 | 44.504 | 1.00 | 44.58 | C |
| ATOM | 4062 | CD2 | PHE | B | 158 | 17.981 | 8.003 | 42.215 | 1.00 | 45.69 | C |
| ATOM | 4063 | CE2 | PHE | B | 158 | 17.233 | 8.139 | 43.377 | 1.00 | 46.91 | C |
| ATOM | 4064 | C | PHE | B | 158 | 22.129 | 7.630 | 39.884 | 1.00 | 45.66 | C |
| ATOM | 4065 | O | PHE | B | 158 | 22.747 | 6.560 | 39.999 | 1.00 | 46.94 | O |
| ATOM | 4066 | N | TRP | B | 159 | 22.359 | 8.514 | 38.906 | 1.00 | 44.23 | N |
| ATOM | 4067 | CA | TRP | B | 159 | 23.161 | 8.171 | 37.701 | 1.00 | 40.17 | C |
| ATOM | 4068 | CB | TRP | B | 159 | 23.294 | 9.357 | 36.731 | 1.00 | 39.65 | C |
| ATOM | 4069 | CG | TRP | B | 159 | 24.262 | 10.421 | 37.133 | 1.00 | 38.10 | C |
| ATOM | 4070 | CD1 | TRP | B | 159 | 23.977 | 11.584 | 37.792 | 1.00 | 36.66 | C |
| ATOM | 4071 | NE1 | TRP | B | 159 | 25.123 | 12.315 | 37.975 | 1.00 | 36.31 | N |
| ATOM | 4072 | CE2 | TRP | B | 159 | 26.185 | 11.637 | 37.439 | 1.00 | 33.99 | C |
| ATOM | 4073 | CD2 | TRP | B | 159 | 25.677 | 10.445 | 36.864 | 1.00 | 36.15 | C |
| ATOM | 4074 | CE3 | TRP | B | 159 | 26.568 | 9.569 | 36.230 | 1.00 | 35.32 | C |
| ATOM | 4075 | CZ3 | TRP | B | 159 | 27.912 | 9.903 | 36.194 | 1.00 | 36.18 | C |
| ATOM | 4076 | CH2 | TRP | B | 159 | 28.385 | 11.096 | 36.760 | 1.00 | 35.66 | C |
| ATOM | 4077 | CZ2 | TRP | B | 159 | 27.535 | 11.981 | 37.387 | 1.00 | 33.26 | C |
| ATOM | 4078 | C | TRP | B | 159 | 24.524 | 7.611 | 38.113 | 1.00 | 41.03 | C |
| ATOM | 4079 | O | TRP | B | 159 | 24.965 | 6.603 | 37.541 | 1.00 | 42.62 | O |
| ATOM | 4080 | N | CYS | B | 160 | 25.193 | 8.244 | 39.060 | 1.00 | 40.32 | N |
| ATOM | 4081 | CA | CYS | B | 160 | 26.547 | 7.826 | 39.484 | 1.00 | 43.42 | C |
| ATOM | 4082 | CB | CYS | B | 160 | 27.147 | 8.860 | 40.430 | 1.00 | 47.68 | C |
| ATOM | 4083 | SG | CYS | B | 160 | 28.804 | 8.389 | 40.979 | 1.00 | 67.73 | S |
| ATOM | 4084 | C | CYS | B | 160 | 26.466 | 6.434 | 40.143 | 1.00 | 44.40 | C |
| ATOM | 4085 | O | CYS | B | 160 | 27.416 | 5.621 | 39.992 | 1.00 | 44.01 | O |
| ATOM | 4086 | N | ALA | B | 161 | 25.390 | 6.174 | 40.883 | 1.00 | 40.31 | N |
| ATOM | 4087 | CA | ALA | B | 161 | 25.221 | 4.951 | 41.688 | 1.00 | 41.15 | C |
| ATOM | 4088 | CB | ALA | B | 161 | 24.061 | 5.113 | 42.643 | 1.00 | 40.88 | C |
| ATOM | 4089 | C | ALA | B | 161 | 25.023 | 3.786 | 40.718 | 1.00 | 42.65 | C |
| ATOM | 4090 | O | ALA | B | 161 | 25.691 | 2.757 | 40.882 | 1.00 | 48.83 | O |
| ATOM | 4091 | N | LEU | B | 162 | 24.177 | 3.988 | 39.707 | 1.00 | 43.26 | N |
| ATOM | 4092 | CA | LEU | B | 162 | 23.816 | 2.969 | 38.688 | 1.00 | 43.41 | C |
| ATOM | 4093 | CB | LEU | B | 162 | 22.565 | 3.440 | 37.946 | 1.00 | 42.70 | C |
| ATOM | 4094 | CG | LEU | B | 162 | 21.328 | 3.564 | 38.831 | 1.00 | 42.33 | C |
| ATOM | 4095 | CD1 | LEU | B | 162 | 20.156 | 4.182 | 38.092 | 1.00 | 45.34 | C |
| ATOM | 4096 | CD2 | LEU | B | 162 | 20.934 | 2.201 | 39.365 | 1.00 | 44.51 | C |
| ATOM | 4097 | C | LEU | B | 162 | 25.007 | 2.704 | 37.757 | 1.00 | 43.32 | C |
| ATOM | 4098 | O | LEU | B | 162 | 25.276 | 1.541 | 37.485 | 1.00 | 47.33 | O |
| ATOM | 4099 | N | VAL | B | 163 | 25.744 | 3.718 | 37.314 | 1.00 | 46.98 | N |
| ATOM | 4100 | CA | VAL | B | 163 | 26.964 | 3.486 | 36.480 | 1.00 | 46.53 | C |
| ATOM | 4101 | CB | VAL | B | 163 | 27.636 | 4.798 | 36.035 | 1.00 | 46.22 | C |
| ATOM | 4102 | CG1 | VAL | B | 163 | 28.977 | 4.556 | 35.353 | 1.00 | 46.14 | C |
| ATOM | 4103 | CG2 | VAL | B | 163 | 26.713 | 5.603 | 35.135 | 1.00 | 47.94 | C |
| ATOM | 4104 | C | VAL | B | 163 | 27.929 | 2.622 | 37.295 | 1.00 | 46.66 | C |
| ATOM | 4105 | O | VAL | B | 163 | 28.636 | 1.760 | 36.704 | 1.00 | 45.41 | O |
| ATOM | 4106 | N | GLU | B | 164 | 27.985 | 2.861 | 38.603 | 1.00 | 45.54 | N |
| ATOM | 4107 | CA | GLU | B | 164 | 28.890 | 2.111 | 39.507 | 1.00 | 45.95 | C |
| ATOM | 4108 | CB | GLU | B | 164 | 28.912 | 2.719 | 40.898 | 1.00 | 46.36 | C |
| ATOM | 4109 | CG | GLU | B | 164 | 30.071 | 2.209 | 41.727 | 1.00 | 44.63 | C |
| ATOM | 4110 | CD | GLU | B | 164 | 29.950 | 2.622 | 43.178 | 1.00 | 43.38 | C |
| ATOM | 4111 | OE1 | GLU | B | 164 | 28.782 | 2.880 | 43.623 | 1.00 | 39.52 | O |
| ATOM | 4112 | OE2 | GLU | B | 164 | 31.008 | 2.721 | 43.839 | 1.00 | 40.05 | O |
| ATOM | 4113 | C | GLU | B | 164 | 28.426 | 0.652 | 39.633 | 1.00 | 45.03 | C |
| ATOM | 4114 | O | GLU | B | 164 | 29.280 | −0.243 | 39.501 | 1.00 | 43.95 | O |
| ATOM | 4115 | N | LYS | B | 165 | 27.142 | 0.408 | 39.901 | 1.00 | 39.25 | N |
| ATOM | 4116 | CA | LYS | B | 165 | 26.627 | −0.982 | 39.969 | 1.00 | 46.78 | C |
| ATOM | 4117 | CB | LYS | B | 165 | 25.110 | −0.998 | 40.191 | 1.00 | 45.52 | C |
| ATOM | 4118 | CG | LYS | B | 165 | 24.598 | −2.341 | 40.681 | 1.00 | 46.33 | C |
| ATOM | 4119 | CD | LYS | B | 165 | 23.127 | −2.574 | 40.451 | 1.00 | 47.62 | C |
| ATOM | 4120 | CE | LYS | B | 165 | 22.713 | −3.963 | 40.871 | 1.00 | 46.10 | C |
| ATOM | 4121 | NZ | LYS | B | 165 | 21.248 | −4.066 | 41.116 | 1.00 | 46.44 | N |
| ATOM | 4122 | C | LYS | B | 165 | 27.052 | −1.708 | 38.677 | 1.00 | 44.79 | C |
| ATOM | 4123 | O | LYS | B | 165 | 27.783 | −2.716 | 38.750 | 1.00 | 42.45 | O |
| ATOM | 4124 | N | ALA | B | 166 | 26.657 | −1.168 | 37.525 | 1.00 | 47.40 | N |
| ATOM | 4125 | CA | ALA | B | 166 | 27.031 | −1.662 | 36.176 | 1.00 | 45.26 | C |
| ATOM | 4126 | CB | ALA | B | 166 | 26.598 | −0.666 | 35.139 | 1.00 | 47.30 | C |
| ATOM | 4127 | C | ALA | B | 166 | 28.538 | −1.916 | 36.115 | 1.00 | 43.29 | C |
| ATOM | 4128 | O | ALA | B | 166 | 28.949 | −3.012 | 35.721 | 1.00 | 46.63 | O |
| ATOM | 4129 | N | TYR | B | 167 | 29.352 | −0.954 | 36.519 | 1.00 | 40.43 | N |
| ATOM | 4130 | CA | TYR | B | 167 | 30.820 | −1.134 | 36.486 | 1.00 | 41.92 | C |
| ATOM | 4131 | CB | TYR | B | 167 | 31.569 | 0.142 | 36.879 | 1.00 | 41.64 | C |
| ATOM | 4132 | CG | TYR | B | 167 | 32.975 | 0.220 | 36.340 | 1.00 | 38.87 | C |
| ATOM | 4133 | CD1 | TYR | B | 167 | 33.217 | 0.181 | 34.981 | 1.00 | 39.80 | C |
| ATOM | 4134 | CE1 | TYR | B | 167 | 34.496 | 0.284 | 34.466 | 1.00 | 40.00 | C |
| ATOM | 4135 | CZ | TYR | B | 167 | 35.574 | 0.442 | 35.308 | 1.00 | 41.60 | C |
| ATOM | 4136 | OH | TYR | B | 167 | 36.845 | 0.499 | 34.798 | 1.00 | 42.66 | O |
| ATOM | 4137 | CE2 | TYR | B | 167 | 35.352 | 0.466 | 36.674 | 1.00 | 45.35 | C |
| ATOM | 4138 | CD2 | TYR | B | 167 | 34.061 | 0.366 | 37.171 | 1.00 | 41.08 | C |
| ATOM | 4139 | C | TYR | B | 167 | 31.190 | −2.293 | 37.410 | 1.00 | 44.51 | C |
| ATOM | 4140 | O | TYR | B | 167 | 32.149 | −3.030 | 37.086 | 1.00 | 45.61 | O |
| ATOM | 4141 | N | ALA | B | 168 | 30.477 | −2.432 | 38.535 | 1.00 | 45.05 | N |
| ATOM | 4142 | CA | ALA | B | 168 | 30.746 | −3.497 | 39.533 | 1.00 | 46.93 | C |
| ATOM | 4143 | CB | ALA | B | 168 | 29.951 | −3.316 | 40.799 | 1.00 | 45.60 | C |
| ATOM | 4144 | C | ALA | B | 168 | 30.471 | −4.858 | 38.890 | 1.00 | 42.56 | C |
| ATOM | 4145 | O | ALA | B | 168 | 31.382 | −5.669 | 38.931 | 1.00 | 51.05 | O |
| ATOM | 4146 | N | LYS | B | 169 | 29.306 | −5.059 | 38.263 | 1.00 | 41.22 | N |
| ATOM | 4147 | CA | LYS | B | 169 | 28.963 | −6.295 | 37.491 | 1.00 | 46.88 | C |
| ATOM | 4148 | CB | LYS | B | 169 | 27.701 | −6.070 | 36.669 | 1.00 | 46.04 | C |
| ATOM | 4149 | CG | LYS | B | 169 | 27.182 | −7.291 | 35.926 | 1.00 | 49.41 | C |
| ATOM | 4150 | CD | LYS | B | 169 | 25.771 | −7.046 | 35.428 | 1.00 | 50.07 | C |
| ATOM | 4151 | CE | LYS | B | 169 | 25.144 | −8.213 | 34.703 | 1.00 | 49.53 | C |
| ATOM | 4152 | NZ | LYS | B | 169 | 23.722 | −7.929 | 34.383 | 1.00 | 49.78 | N |
| ATOM | 4153 | C | LYS | B | 169 | 30.098 | −6.706 | 36.539 | 1.00 | 52.00 | C |
| ATOM | 4154 | O | LYS | B | 169 | 30.417 | −7.901 | 36.472 | 1.00 | 56.82 | O |
| ATOM | 4155 | N | LEU | B | 170 | 30.699 | −5.734 | 35.853 | 1.00 | 56.22 | N |
| ATOM | 4156 | CA | LEU | B | 170 | 31.830 | −5.919 | 34.910 | 1.00 | 56.58 | C |
| ATOM | 4157 | CB | LEU | B | 170 | 32.076 | −4.583 | 34.197 | 1.00 | 63.10 | C |
| ATOM | 4158 | CG | LEU | B | 170 | 32.838 | −4.641 | 32.880 | 1.00 | 63.32 | C |
| ATOM | 4159 | CD1 | LEU | B | 170 | 32.226 | −5.675 | 31.949 | 1.00 | 66.02 | C |
| ATOM | 4160 | CD2 | LEU | B | 170 | 32.860 | −3.265 | 32.228 | 1.00 | 61.44 | C |
| ATOM | 4161 | C | LEU | B | 170 | 33.076 | −6.372 | 35.672 | 1.00 | 54.19 | C |
| ATOM | 4162 | O | LEU | B | 170 | 33.913 | −7.082 | 35.081 | 1.00 | 54.34 | O |
| ATOM | 4163 | N | ALA | B | 171 | 33.253 | −5.921 | 36.910 | 1.00 | 49.89 | N |
| ATOM | 4164 | CA | ALA | B | 171 | 34.426 | −6.322 | 37.724 | 1.00 | 51.21 | C |
| ATOM | 4165 | CB | ALA | B | 171 | 34.820 | −5.243 | 38.714 | 1.00 | 50.85 | C |
| ATOM | 4166 | C | ALA | B | 171 | 34.107 | −7.658 | 38.406 | 1.00 | 50.27 | C |
| ATOM | 4167 | O | ALA | B | 171 | 35.080 | −8.371 | 38.742 | 1.00 | 49.29 | O |
| ATOM | 4168 | N | GLY | B | 172 | 32.812 | −7.975 | 38.574 | 1.00 | 47.49 | N |
| ATOM | 4169 | CA | GLY | B | 172 | 32.311 | −9.222 | 39.191 | 1.00 | 57.99 | C |
| ATOM | 4170 | C | GLY | B | 172 | 31.544 | −8.973 | 40.484 | 1.00 | 59.79 | C |
| ATOM | 4171 | O | GLY | B | 172 | 30.515 | −9.642 | 40.690 | 1.00 | 65.29 | O |
| ATOM | 4172 | N | CYS | B | 173 | 32.049 | −8.069 | 41.334 | 1.00 | 60.71 | N |
| ATOM | 4173 | CA | CYS | B | 173 | 31.406 | −7.583 | 42.591 | 1.00 | 53.41 | C |
| ATOM | 4174 | CB | CYS | B | 173 | 31.645 | −8.557 | 43.740 | 1.00 | 43.15 | C |
| ATOM | 4175 | SG | CYS | B | 173 | 33.385 | −8.616 | 44.247 | 1.00 | 46.95 | S |
| ATOM | 4176 | C | CYS | B | 173 | 31.986 | −6.209 | 42.972 | 1.00 | 52.29 | C |
| ATOM | 4177 | O | CYS | B | 173 | 33.128 | −5.888 | 42.506 | 1.00 | 51.04 | O |
| ATOM | 4178 | N | TYR | B | 174 | 31.279 | −5.467 | 43.834 | 1.00 | 44.67 | N |
| ATOM | 4179 | CA | TYR | B | 174 | 31.679 | −4.107 | 44.290 | 1.00 | 43.86 | C |
| ATOM | 4180 | CB | TYR | B | 174 | 30.709 | −3.560 | 45.328 | 1.00 | 38.79 | C |
| ATOM | 4181 | CG | TYR | B | 174 | 29.512 | −2.844 | 44.760 | 1.00 | 35.55 | C |
| ATOM | 4182 | CD1 | TYR | B | 174 | 28.374 | −3.530 | 44.386 | 1.00 | 33.92 | C |
| ATOM | 4183 | CE1 | TYR | B | 174 | 27.243 | −2.867 | 43.934 | 1.00 | 36.10 | C |
| ATOM | 4184 | CZ | TYR | B | 174 | 27.259 | −1.490 | 43.806 | 1.00 | 36.23 | C |
| ATOM | 4185 | OH | TYR | B | 174 | 26.167 | −0.817 | 43.333 | 1.00 | 43.20 | O |
| ATOM | 4186 | CE2 | TYR | B | 174 | 28.385 | −0.790 | 44.180 | 1.00 | 34.99 | C |
| ATOM | 4187 | CD2 | TYR | B | 174 | 29.497 | −1.466 | 44.650 | 1.00 | 35.17 | C |
| ATOM | 4188 | C | TYR | B | 174 | 33.107 | −4.122 | 44.843 | 1.00 | 47.03 | C |
| ATOM | 4189 | O | TYR | B | 174 | 33.889 | −3.238 | 44.488 | 1.00 | 54.00 | O |
| ATOM | 4190 | N | GLN | B | 175 | 33.473 | −5.134 | 45.623 | 1.00 | 50.85 | N |
| ATOM | 4191 | CA | GLN | B | 175 | 34.804 | −5.187 | 46.290 | 1.00 | 56.01 | C |
| ATOM | 4192 | CB | GLN | B | 175 | 34.891 | −6.388 | 47.239 | 1.00 | 56.83 | C |
| ATOM | 4193 | CG | GLN | B | 175 | 36.100 | −6.343 | 48.169 | 1.00 | 59.78 | C |
| ATOM | 4194 | CD | GLN | B | 175 | 36.187 | −7.519 | 49.116 | 1.00 | 58.77 | C |
| ATOM | 4195 | OE1 | GLN | B | 175 | 35.226 | −8.253 | 49.336 | 1.00 | 66.53 | O |
| ATOM | 4196 | NE2 | GLN | B | 175 | 37.359 | −7.715 | 49.688 | 1.00 | 57.44 | N |
| ATOM | 4197 | C | GLN | B | 175 | 35.912 | −5.202 | 45.228 | 1.00 | 51.40 | C |
| ATOM | 4198 | O | GLN | B | 175 | 37.036 | −4.776 | 45.539 | 1.00 | 53.30 | O |
| ATOM | 4199 | N | ALA | B | 176 | 35.630 | −5.686 | 44.019 | 1.00 | 53.42 | N |
| ATOM | 4200 | CA | ALA | B | 176 | 36.633 | −5.754 | 42.928 | 1.00 | 54.97 | C |
| ATOM | 4201 | CB | ALA | B | 176 | 36.096 | −6.583 | 41.786 | 1.00 | 55.62 | C |
| ATOM | 4202 | C | ALA | B | 176 | 37.024 | −4.328 | 42.496 | 1.00 | 53.89 | C |
| ATOM | 4203 | O | ALA | B | 176 | 38.210 | −4.119 | 42.135 | 1.00 | 46.50 | O |
| ATOM | 4204 | N | LEU | B | 177 | 36.094 | −3.367 | 42.600 | 1.00 | 49.54 | N |
| ATOM | 4205 | CA | LEU | B | 177 | 36.371 | −1.930 | 42.324 | 1.00 | 51.57 | C |
| ATOM | 4206 | CB | LEU | B | 177 | 35.086 | −1.105 | 42.442 | 1.00 | 51.06 | C |
| ATOM | 4207 | CG | LEU | B | 177 | 33.963 | −1.513 | 41.486 | 1.00 | 53.48 | C |
| ATOM | 4208 | CD1 | LEU | B | 177 | 32.754 | −0.599 | 41.613 | 1.00 | 55.79 | C |
| ATOM | 4209 | CD2 | LEU | B | 177 | 34.458 | −1.530 | 40.047 | 1.00 | 53.10 | C |
| ATOM | 4210 | C | LEU | B | 177 | 37.476 | −1.384 | 43.245 | 1.00 | 56.97 | C |
| ATOM | 4211 | O | LEU | B | 177 | 38.149 | −0.441 | 42.789 | 1.00 | 60.41 | O |
| ATOM | 4212 | N | ASP | B | 178 | 37.731 | −1.949 | 44.440 | 1.00 | 49.47 | N |
| ATOM | 4213 | CA | ASP | B | 178 | 38.812 | −1.425 | 45.330 | 1.00 | 47.22 | C |
| ATOM | 4214 | CB | ASP | B | 178 | 38.831 | −2.092 | 46.703 | 1.00 | 50.80 | C |
| ATOM | 4215 | CG | ASP | B | 178 | 37.466 | −2.134 | 47.388 | 1.00 | 59.11 | C |
| ATOM | 4216 | OD1 | ASP | B | 178 | 36.483 | −1.516 | 46.862 | 1.00 | 57.36 | O |
| ATOM | 4217 | OD2 | ASP | B | 178 | 37.378 | −2.809 | 48.427 | 1.00 | 59.98 | O |
| ATOM | 4218 | C | ASP | B | 178 | 40.166 | −1.557 | 44.629 | 1.00 | 48.47 | C |
| ATOM | 4219 | O | ASP | B | 178 | 41.069 | −0.788 | 44.970 | 1.00 | 47.80 | O |
| ATOM | 4220 | N | GLY | B | 179 | 40.290 | −2.479 | 43.668 | 1.00 | 52.35 | N |
| ATOM | 4221 | CA | GLY | B | 179 | 41.530 | −2.719 | 42.903 | 1.00 | 57.68 | C |
| ATOM | 4222 | C | GLY | B | 179 | 41.664 | −1.827 | 41.672 | 1.00 | 59.50 | C |
| ATOM | 4223 | O | GLY | B | 179 | 42.756 | −1.805 | 41.096 | 1.00 | 60.52 | O |
| ATOM | 4224 | N | GLY | B | 180 | 40.608 | −1.104 | 41.285 | 1.00 | 61.12 | N |
| ATOM | 4225 | CA | GLY | B | 180 | 40.566 | −0.265 | 40.066 | 1.00 | 60.52 | C |
| ATOM | 4226 | C | GLY | B | 180 | 41.319 | 1.054 | 40.212 | 1.00 | 59.61 | C |
| ATOM | 4227 | O | GLY | B | 180 | 41.843 | 1.338 | 41.315 | 1.00 | 62.41 | O |
| ATOM | 4228 | N | ASN | B | 181 | 41.359 | 1.843 | 39.135 | 1.00 | 58.10 | N |
| ATOM | 4229 | CA | ASN | B | 181 | 42.013 | 3.179 | 39.084 | 1.00 | 59.70 | C |
| ATOM | 4230 | CB | ASN | B | 181 | 43.523 | 3.063 | 38.849 | 1.00 | 59.96 | C |
| ATOM | 4231 | CG | ASN | B | 181 | 43.864 | 2.445 | 37.508 | 1.00 | 62.60 | C |
| ATOM | 4232 | OD1 | ASN | B | 181 | 43.719 | 3.079 | 36.468 | 1.00 | 58.03 | O |
| ATOM | 4233 | ND2 | ASN | B | 181 | 44.314 | 1.203 | 37.520 | 1.00 | 64.41 | N |
| ATOM | 4234 | C | ASN | B | 181 | 41.338 | 4.041 | 38.005 | 1.00 | 57.71 | C |
| ATOM | 4235 | O | ASN | B | 181 | 40.721 | 3.479 | 37.066 | 1.00 | 49.10 | O |
| ATOM | 4236 | N | THR | B | 182 | 41.486 | 5.361 | 38.127 | 1.00 | 49.73 | N |
| ATOM | 4237 | CA | THR | B | 182 | 40.867 | 6.372 | 37.237 | 1.00 | 47.98 | C |
| ATOM | 4238 | CB | THR | B | 182 | 41.060 | 7.762 | 37.862 | 1.00 | 52.37 | C |
| ATOM | 4239 | OG1 | THR | B | 182 | 40.467 | 7.797 | 39.170 | 1.00 | 48.32 | O |
| ATOM | 4240 | CG2 | THR | B | 182 | 40.451 | 8.859 | 37.016 | 1.00 | 51.46 | C |
| ATOM | 4241 | C | THR | B | 182 | 41.396 | 6.190 | 35.794 | 1.00 | 44.34 | C |
| ATOM | 4242 | O | THR | B | 182 | 40.591 | 6.207 | 34.861 | 1.00 | 38.72 | O |
| ATOM | 4243 | N | ALA | B | 183 | 42.696 | 5.988 | 35.582 | 1.00 | 44.74 | N |
| ATOM | 4244 | CA | ALA | B | 183 | 43.259 | 5.804 | 34.222 | 1.00 | 48.78 | C |
| ATOM | 4245 | CB | ALA | B | 183 | 44.725 | 5.450 | 34.298 | 1.00 | 50.37 | C |
| ATOM | 4246 | C | ALA | B | 183 | 42.448 | 4.731 | 33.472 | 1.00 | 53.41 | C |
| ATOM | 4247 | O | ALA | B | 183 | 41.847 | 5.051 | 32.422 | 1.00 | 50.80 | O |
| ATOM | 4248 | N | ASP | B | 184 | 42.392 | 3.513 | 34.021 | 1.00 | 55.64 | N |
| ATOM | 4249 | CA | ASP | B | 184 | 41.724 | 2.333 | 33.408 | 1.00 | 54.28 | C |
| ATOM | 4250 | CB | ASP | B | 184 | 41.990 | 1.043 | 34.189 | 1.00 | 58.26 | C |
| ATOM | 4251 | CG | ASP | B | 184 | 43.452 | 0.619 | 34.174 | 1.00 | 63.59 | C |
| ATOM | 4252 | OD1 | ASP | B | 184 | 44.215 | 1.118 | 33.311 | 1.00 | 64.98 | O |
| ATOM | 4253 | OD2 | ASP | B | 184 | 43.825 | −0.194 | 35.036 | 1.00 | 70.56 | O |
| ATOM | 4254 | C | ASP | B | 184 | 40.228 | 2.599 | 33.260 | 1.00 | 50.57 | C |
| ATOM | 4255 | O | ASP | B | 184 | 39.663 | 2.110 | 32.283 | 1.00 | 61.11 | O |
| ATOM | 4256 | N | ALA | B | 185 | 39.600 | 3.357 | 34.155 | 1.00 | 46.45 | N |
| ATOM | 4257 | CA | ALA | B | 185 | 38.146 | 3.637 | 34.060 | 1.00 | 42.78 | C |
| ATOM | 4258 | CB | ALA | B | 185 | 37.604 | 4.196 | 35.343 | 1.00 | 40.92 | C |
| ATOM | 4259 | C | ALA | B | 185 | 37.877 | 4.595 | 32.899 | 1.00 | 42.24 | C |
| ATOM | 4260 | O | ALA | B | 185 | 36.804 | 4.474 | 32.261 | 1.00 | 45.68 | O |
| ATOM | 4261 | N | LEU | B | 186 | 38.770 | 5.553 | 32.668 | 1.00 | 41.87 | N |
| ATOM | 4262 | CA | LEU | B | 186 | 38.551 | 6.558 | 31.600 | 1.00 | 46.88 | C |
| ATOM | 4263 | CB | LEU | B | 186 | 39.582 | 7.688 | 31.696 | 1.00 | 46.10 | C |
| ATOM | 4264 | CG | LEU | B | 186 | 39.406 | 8.592 | 32.922 | 1.00 | 46.16 | C |
| ATOM | 4265 | CD1 | LEU | B | 186 | 40.338 | 9.785 | 32.858 | 1.00 | 45.40 | C |
| ATOM | 4266 | CD2 | LEU | B | 186 | 37.958 | 9.045 | 33.086 | 1.00 | 46.09 | C |
| ATOM | 4267 | C | LEU | B | 186 | 38.580 | 5.807 | 30.271 | 1.00 | 47.11 | C |
| ATOM | 4268 | O | LEU | B | 186 | 37.633 | 5.985 | 29.474 | 1.00 | 41.97 | O |
| ATOM | 4269 | N | VAL | B | 187 | 39.533 | 4.881 | 30.128 | 1.00 | 49.80 | N |
| ATOM | 4270 | CA | VAL | B | 187 | 39.645 | 3.998 | 28.933 | 1.00 | 47.02 | C |
| ATOM | 4271 | CB | VAL | B | 187 | 40.889 | 3.097 | 29.015 | 1.00 | 49.38 | C |
| ATOM | 4272 | CG1 | VAL | B | 187 | 40.950 | 2.119 | 27.853 | 1.00 | 50.36 | C |
| ATOM | 4273 | CG2 | VAL | B | 187 | 42.174 | 3.922 | 29.078 | 1.00 | 50.26 | C |
| ATOM | 4274 | C | VAL | B | 187 | 38.326 | 3.224 | 28.782 | 1.00 | 48.59 | C |
| ATOM | 4275 | O | VAL | B | 187 | 37.751 | 3.266 | 27.678 | 1.00 | 59.14 | O |
| ATOM | 4276 | N | ASP | B | 188 | 37.794 | 2.630 | 29.851 | 1.00 | 46.83 | N |
| ATOM | 4277 | CA | ASP | B | 188 | 36.543 | 1.827 | 29.768 | 1.00 | 45.48 | C |
| ATOM | 4278 | CB | ASP | B | 188 | 36.280 | 0.994 | 31.027 | 1.00 | 48.55 | C |
| ATOM | 4279 | CG | ASP | B | 188 | 37.393 | 0.015 | 31.369 | 1.00 | 50.97 | C |
| ATOM | 4280 | OD1 | ASP | B | 188 | 38.139 | −0.393 | 30.446 | 1.00 | 53.61 | O |
| ATOM | 4281 | OD2 | ASP | B | 188 | 37.522 | −0.319 | 32.555 | 1.00 | 55.22 | O |
| ATOM | 4282 | C | ASP | B | 188 | 35.355 | 2.735 | 29.467 | 1.00 | 44.26 | C |
| ATOM | 4283 | O | ASP | B | 188 | 34.348 | 2.201 | 29.011 | 1.00 | 52.83 | O |
| ATOM | 4284 | N | PHE | B | 189 | 35.446 | 4.041 | 29.706 | 1.00 | 48.59 | N |
| ATOM | 4285 | CA | PHE | B | 189 | 34.315 | 4.986 | 29.483 | 1.00 | 54.23 | C |
| ATOM | 4286 | CB | PHE | B | 189 | 34.324 | 6.114 | 30.524 | 1.00 | 61.54 | C |
| ATOM | 4287 | CG | PHE | B | 189 | 33.906 | 5.778 | 31.939 | 1.00 | 59.57 | C |
| ATOM | 4288 | CD1 | PHE | B | 189 | 33.431 | 4.527 | 32.289 | 1.00 | 62.60 | C |
| ATOM | 4289 | CE1 | PHE | B | 189 | 33.020 | 4.257 | 33.583 | 1.00 | 60.93 | C |
| ATOM | 4290 | CZ | PHE | B | 189 | 33.065 | 5.237 | 34.541 | 1.00 | 61.28 | C |
| ATOM | 4291 | CD2 | PHE | B | 189 | 33.931 | 6.759 | 32.915 | 1.00 | 55.63 | C |
| ATOM | 4292 | CE2 | PHE | B | 189 | 33.535 | 6.482 | 34.212 | 1.00 | 59.65 | C |
| ATOM | 4293 | C | PHE | B | 189 | 34.368 | 5.603 | 28.073 | 1.00 | 56.06 | C |
| ATOM | 4294 | O | PHE | B | 189 | 33.298 | 6.031 | 27.589 | 1.00 | 54.32 | O |
| ATOM | 4295 | N | THR | B | 190 | 35.549 | 5.672 | 27.440 | 1.00 | 55.02 | N |
| ATOM | 4296 | CA | THR | B | 190 | 35.794 | 6.520 | 26.237 | 1.00 | 55.87 | C |
| ATOM | 4297 | CB | THR | B | 190 | 36.839 | 7.592 | 26.545 | 1.00 | 52.27 | C |
| ATOM | 4298 | OG1 | THR | B | 190 | 38.096 | 6.965 | 26.810 | 1.00 | 47.67 | O |
| ATOM | 4299 | CG2 | THR | B | 190 | 36.426 | 8.458 | 27.715 | 1.00 | 56.43 | C |
| ATOM | 4300 | C | THR | B | 190 | 36.275 | 5.739 | 25.004 | 1.00 | 58.40 | C |
| ATOM | 4301 | O | THR | B | 190 | 36.102 | 6.259 | 23.889 | 1.00 | 68.39 | O |
| ATOM | 4302 | N | GLY | B | 191 | 36.902 | 4.579 | 25.184 | 1.00 | 55.23 | N |
| ATOM | 4303 | CA | GLY | B | 191 | 37.602 | 3.853 | 24.110 | 1.00 | 54.71 | C |
| ATOM | 4304 | C | GLY | B | 191 | 39.032 | 4.337 | 23.960 | 1.00 | 54.77 | C |
| ATOM | 4305 | O | GLY | B | 191 | 39.789 | 3.758 | 23.157 | 1.00 | 54.83 | O |
| ATOM | 4306 | N | GLY | B | 192 | 39.414 | 5.344 | 24.738 | 1.00 | 54.31 | N |
| ATOM | 4307 | CA | GLY | B | 192 | 40.655 | 6.095 | 24.515 | 1.00 | 53.28 | C |
| ATOM | 4308 | C | GLY | B | 192 | 41.863 | 5.317 | 24.964 | 1.00 | 50.18 | C |
| ATOM | 4309 | O | GLY | B | 192 | 41.774 | 4.097 | 25.105 | 1.00 | 63.48 | O |
| ATOM | 4310 | N | VAL | B | 193 | 42.956 | 6.031 | 25.182 | 1.00 | 51.07 | N |
| ATOM | 4311 | CA | VAL | B | 193 | 44.243 | 5.477 | 25.668 | 1.00 | 57.81 | C |
| ATOM | 4312 | CB | VAL | B | 193 | 45.240 | 5.333 | 24.506 | 1.00 | 61.60 | C |
| ATOM | 4313 | CG1 | VAL | B | 193 | 46.677 | 5.218 | 24.993 | 1.00 | 59.95 | C |
| ATOM | 4314 | CG2 | VAL | B | 193 | 44.858 | 4.160 | 23.621 | 1.00 | 65.90 | C |
| ATOM | 4315 | C | VAL | B | 193 | 44.756 | 6.416 | 26.756 | 1.00 | 63.34 | C |
| ATOM | 4316 | O | VAL | B | 193 | 44.770 | 7.648 | 26.535 | 1.00 | 68.84 | O |
| ATOM | 4317 | N | SER | B | 194 | 45.130 | 5.851 | 27.897 | 1.00 | 65.76 | N |
| ATOM | 4318 | CA | SER | B | 194 | 45.616 | 6.612 | 29.069 | 1.00 | 71.45 | C |
| ATOM | 4319 | CB | SER | B | 194 | 45.106 | 6.021 | 30.359 | 1.00 | 77.34 | C |
| ATOM | 4320 | OG | SER | B | 194 | 43.745 | 6.394 | 30.578 | 1.00 | 79.41 | O |
| ATOM | 4321 | C | SER | B | 194 | 47.141 | 6.682 | 28.998 | 1.00 | 72.16 | C |
| ATOM | 4322 | O | SER | B | 194 | 47.760 | 5.795 | 28.383 | 1.00 | 78.29 | O |
| ATOM | 4323 | N | GLU | B | 195 | 47.712 | 7.752 | 29.534 | 1.00 | 70.70 | N |
| ATOM | 4324 | CA | GLU | B | 195 | 49.173 | 7.906 | 29.694 | 1.00 | 79.96 | C |
| ATOM | 4325 | CB | GLU | B | 195 | 49.749 | 8.748 | 28.557 | 1.00 | 89.01 | C |
| ATOM | 4326 | CG | GLU | B | 195 | 51.218 | 9.102 | 28.748 | 1.00 | 92.10 | C |
| ATOM | 4327 | CD | GLU | B | 195 | 51.949 | 9.549 | 27.493 | 1.00 | 96.81 | C |
| ATOM | 4328 | OE1 | GLU | B | 195 | 53.192 | 9.668 | 27.553 | 1.00 | 102.39 | O |
| ATOM | 4329 | OE2 | GLU | B | 195 | 51.280 | 9.767 | 26.459 | 1.00 | 95.65 | O |
| ATOM | 4330 | C | GLU | B | 195 | 49.405 | 8.557 | 31.047 | 1.00 | 85.54 | C |
| ATOM | 4331 | O | GLU | B | 195 | 49.243 | 9.764 | 31.178 | 1.00 | 89.07 | O |
| ATOM | 4332 | N | PRO | B | 196 | 49.715 | 7.779 | 32.106 | 1.00 | 93.36 | N |
| ATOM | 4333 | CA | PRO | B | 196 | 49.983 | 8.366 | 33.416 | 1.00 | 96.63 | C |
| ATOM | 4334 | CB | PRO | B | 196 | 49.946 | 7.161 | 34.377 | 1.00 | 100.63 | C |
| ATOM | 4335 | CG | PRO | B | 196 | 50.266 | 5.958 | 33.510 | 1.00 | 95.92 | C |
| ATOM | 4336 | CD | PRO | B | 196 | 49.783 | 6.309 | 32.116 | 1.00 | 95.23 | C |
| ATOM | 4337 | C | PRO | B | 196 | 51.332 | 9.103 | 33.430 | 1.00 | 90.47 | C |
| ATOM | 4338 | O | PRO | B | 196 | 52.323 | 8.539 | 33.013 | 1.00 | 90.52 | O |
| ATOM | 4339 | N | ILE | B | 197 | 51.329 | 10.353 | 33.896 | 1.00 | 87.57 | N |
| ATOM | 4340 | CA | ILE | B | 197 | 52.556 | 11.168 | 34.115 | 1.00 | 79.98 | C |
| ATOM | 4341 | CB | ILE | B | 197 | 52.449 | 12.515 | 33.371 | 1.00 | 82.44 | C |
| ATOM | 4342 | CG1 | ILE | B | 197 | 52.435 | 12.318 | 31.853 | 1.00 | 84.44 | C |
| ATOM | 4343 | CG2 | ILE | B | 197 | 53.553 | 13.471 | 33.790 | 1.00 | 87.05 | C |
| ATOM | 4344 | CD1 | ILE | B | 197 | 51.061 | 12.401 | 31.240 | 1.00 | 86.89 | C |
| ATOM | 4345 | C | ILE | B | 197 | 52.745 | 11.310 | 35.628 | 1.00 | 74.69 | C |
| ATOM | 4346 | O | ILE | B | 197 | 51.730 | 11.484 | 36.341 | 1.00 | 68.26 | O |
| ATOM | 4347 | N | ASP | B | 198 | 53.992 | 11.185 | 36.090 | 1.00 | 75.00 | N |
| ATOM | 4348 | CA | ASP | B | 198 | 54.366 | 11.251 | 37.524 | 1.00 | 79.03 | C |
| ATOM | 4349 | CB | ASP | B | 198 | 55.114 | 9.995 | 37.966 | 1.00 | 82.64 | C |
| ATOM | 4350 | CG | ASP | B | 198 | 55.345 | 9.943 | 39.466 | 1.00 | 89.54 | C |
| ATOM | 4351 | OD1 | ASP | B | 198 | 55.378 | 11.026 | 40.095 | 1.00 | 82.87 | O |
| ATOM | 4352 | OD2 | ASP | B | 198 | 55.484 | 8.821 | 39.993 | 1.00 | 104.20 | O |
| ATOM | 4353 | C | ASP | B | 198 | 55.204 | 12.508 | 37.756 | 1.00 | 80.67 | C |
| ATOM | 4354 | O | ASP | B | 198 | 56.353 | 12.548 | 37.283 | 1.00 | 87.72 | O |
| ATOM | 4355 | N | LEU | B | 199 | 54.657 | 13.473 | 38.498 | 1.00 | 85.48 | N |
| ATOM | 4356 | CA | LEU | B | 199 | 55.269 | 14.812 | 38.722 | 1.00 | 84.01 | C |
| ATOM | 4357 | CB | LEU | B | 199 | 54.167 | 15.796 | 39.126 | 1.00 | 79.10 | C |
| ATOM | 4358 | CG | LEU | B | 199 | 52.949 | 15.843 | 38.206 | 1.00 | 78.80 | C |
| ATOM | 4359 | CD1 | LEU | B | 199 | 51.919 | 16.831 | 38.733 | 1.00 | 82.89 | C |
| ATOM | 4360 | CD2 | LEU | B | 199 | 53.341 | 16.193 | 36.778 | 1.00 | 73.11 | C |
| ATOM | 4361 | C | LEU | B | 199 | 56.374 | 14.749 | 39.790 | 1.00 | 84.88 | C |
| ATOM | 4362 | O | LEU | B | 199 | 57.114 | 15.740 | 39.895 | 1.00 | 85.45 | O |
| ATOM | 4363 | N | THR | B | 200 | 56.499 | 13.644 | 40.541 | 1.00 | 94.07 | N |
| ATOM | 4364 | CA | THR | B | 200 | 57.500 | 13.475 | 41.636 | 1.00 | 97.74 | C |
| ATOM | 4365 | CB | THR | B | 200 | 56.797 | 13.196 | 42.974 | 1.00 | 94.92 | C |
| ATOM | 4366 | OG1 | THR | B | 200 | 56.206 | 11.897 | 42.942 | 1.00 | 99.15 | O |
| ATOM | 4367 | CG2 | THR | B | 200 | 55.717 | 14.205 | 43.300 | 1.00 | 95.07 | C |
| ATOM | 4368 | C | THR | B | 200 | 58.566 | 12.415 | 41.279 | 1.00 | 106.19 | C |
| ATOM | 4369 | O | THR | B | 200 | 59.545 | 12.325 | 42.049 | 1.00 | 108.30 | O |
| ATOM | 4370 | N | GLU | B | 201 | 58.405 | 11.641 | 40.189 | 1.00 | 110.68 | N |
| ATOM | 4371 | CA | GLU | B | 201 | 59.464 | 10.748 | 39.617 | 1.00 | 108.18 | C |
| ATOM | 4372 | CB | GLU | B | 201 | 58.912 | 9.404 | 39.130 | 1.00 | 100.32 | C |
| ATOM | 4373 | C | GLU | B | 201 | 60.150 | 11.490 | 38.466 | 1.00 | 105.08 | C |
| ATOM | 4374 | O | GLU | B | 201 | 61.371 | 11.345 | 38.319 | 1.00 | 118.39 | O |
| ATOM | 4375 | N | GLY | B | 202 | 59.380 | 12.229 | 37.665 | 1.00 | 101.59 | N |
| ATOM | 4376 | CA | GLY | B | 202 | 59.879 | 13.373 | 36.879 | 1.00 | 110.29 | C |
| ATOM | 4377 | C | GLY | B | 202 | 60.110 | 14.541 | 37.817 | 1.00 | 114.77 | C |
| ATOM | 4378 | O | GLY | B | 202 | 59.685 | 14.419 | 38.987 | 1.00 | 120.09 | O |
| ATOM | 4379 | N | ASP | B | 203 | 60.757 | 15.620 | 37.367 | 1.00 | 107.71 | N |
| ATOM | 4380 | CA | ASP | B | 203 | 61.084 | 16.770 | 38.255 | 1.00 | 115.60 | C |
| ATOM | 4381 | CB | ASP | B | 203 | 62.591 | 16.859 | 38.516 | 1.00 | 117.98 | C |
| ATOM | 4382 | CG | ASP | B | 203 | 62.952 | 17.650 | 39.766 | 1.00 | 115.60 | C |
| ATOM | 4383 | OD1 | ASP | B | 203 | 62.024 | 18.050 | 40.499 | 1.00 | 114.66 | O |
| ATOM | 4384 | OD2 | ASP | B | 203 | 64.160 | 17.860 | 39.999 | 1.00 | 114.34 | O |
| ATOM | 4385 | C | ASP | B | 203 | 60.496 | 18.058 | 37.669 | 1.00 | 116.31 | C |
| ATOM | 4386 | O | ASP | B | 203 | 61.250 | 19.031 | 37.488 | 1.00 | 116.80 | O |
| ATOM | 4387 | N | PHE | B | 204 | 59.178 | 18.074 | 37.450 | 1.00 | 115.37 | N |
| ATOM | 4388 | CA | PHE | B | 204 | 58.437 | 19.157 | 36.747 | 1.00 | 111.35 | C |
| ATOM | 4389 | CB | PHE | B | 204 | 57.020 | 18.678 | 36.409 | 1.00 | 110.82 | C |
| ATOM | 4390 | CG | PHE | B | 204 | 56.978 | 17.610 | 35.344 | 1.00 | 109.08 | C |
| ATOM | 4391 | CD1 | PHE | B | 204 | 57.157 | 16.272 | 35.668 | 1.00 | 107.59 | C |
| ATOM | 4392 | CE1 | PHE | B | 204 | 57.140 | 15.294 | 34.685 | 1.00 | 104.86 | C |
| ATOM | 4393 | CZ | PHE | B | 204 | 56.948 | 15.642 | 33.369 | 1.00 | 108.18 | C |
| ATOM | 4394 | CD2 | PHE | B | 204 | 56.794 | 17.945 | 34.010 | 1.00 | 108.07 | C |
| ATOM | 4395 | CE2 | PHE | B | 204 | 56.775 | 16.965 | 33.029 | 1.00 | 107.43 | C |
| ATOM | 4396 | C | PHE | B | 204 | 58.472 | 20.454 | 37.573 | 1.00 | 107.73 | C |
| ATOM | 4397 | O | PHE | B | 204 | 58.499 | 21.551 | 36.978 | 1.00 | 97.90 | O |
| ATOM | 4398 | N | ALA | B | 205 | 58.495 | 20.338 | 38.904 | 1.00 | 115.12 | N |
| ATOM | 4399 | CA | ALA | B | 205 | 58.547 | 21.475 | 39.854 | 1.00 | 117.23 | C |
| ATOM | 4400 | CB | ALA | B | 205 | 58.477 | 20.962 | 41.274 | 1.00 | 115.59 | C |
| ATOM | 4401 | C | ALA | B | 205 | 59.812 | 22.315 | 39.605 | 1.00 | 115.31 | C |
| ATOM | 4402 | O | ALA | B | 205 | 59.669 | 23.538 | 39.436 | 1.00 | 104.33 | O |
| ATOM | 4403 | N | ASN | B | 206 | 60.991 | 21.682 | 39.546 | 1.00 | 119.27 | N |
| ATOM | 4404 | CA | ASN | B | 206 | 62.315 | 22.365 | 39.459 | 1.00 | 120.90 | C |
| ATOM | 4405 | CB | ASN | B | 206 | 63.222 | 21.972 | 40.629 | 1.00 | 122.32 | C |
| ATOM | 4406 | CG | ASN | B | 206 | 62.539 | 22.100 | 41.975 | 1.00 | 121.68 | C |
| ATOM | 4407 | OD1 | ASN | B | 206 | 62.410 | 23.198 | 42.513 | 1.00 | 112.83 | O |
| ATOM | 4408 | ND2 | ASN | B | 206 | 62.106 | 20.982 | 42.532 | 1.00 | 120.27 | N |
| ATOM | 4409 | C | ASN | B | 206 | 62.985 | 22.064 | 38.108 | 1.00 | 122.64 | C |
| ATOM | 4410 | O | ASN | B | 206 | 64.210 | 21.808 | 38.094 | 1.00 | 116.19 | O |
| ATOM | 4411 | N | ASP | B | 207 | 62.205 | 22.100 | 37.022 | 1.00 | 122.93 | N |
| ATOM | 4412 | CA | ASP | B | 207 | 62.674 | 22.004 | 35.611 | 1.00 | 121.70 | C |
| ATOM | 4413 | CB | ASP | B | 207 | 62.904 | 20.547 | 35.189 | 1.00 | 120.43 | C |
| ATOM | 4414 | CG | ASP | B | 207 | 63.345 | 20.352 | 33.744 | 1.00 | 120.03 | C |
| ATOM | 4415 | OD1 | ASP | B | 207 | 62.605 | 20.784 | 32.839 | 1.00 | 115.58 | O |
| ATOM | 4416 | OD2 | ASP | B | 207 | 64.421 | 19.754 | 33.533 | 1.00 | 111.77 | O |
| ATOM | 4417 | C | ASP | B | 207 | 61.623 | 22.698 | 34.740 | 1.00 | 123.25 | C |
| ATOM | 4418 | O | ASP | B | 207 | 60.513 | 22.142 | 34.613 | 1.00 | 130.37 | O |
| ATOM | 4419 | N | GLU | B | 208 | 61.944 | 23.872 | 34.190 | 1.00 | 116.58 | N |
| ATOM | 4420 | CA | GLU | B | 208 | 60.975 | 24.702 | 33.425 | 1.00 | 119.93 | C |
| ATOM | 4421 | CB | GLU | B | 208 | 61.424 | 26.167 | 33.359 | 1.00 | 119.78 | C |
| ATOM | 4422 | CG | GLU | B | 208 | 60.371 | 27.090 | 32.756 | 1.00 | 122.46 | C |
| ATOM | 4423 | CD | GLU | B | 208 | 60.387 | 28.550 | 33.192 | 1.00 | 123.26 | C |
| ATOM | 4424 | OE1 | GLU | B | 208 | 61.244 | 28.922 | 34.021 | 1.00 | 119.88 | O |
| ATOM | 4425 | OE2 | GLU | B | 208 | 59.527 | 29.316 | 32.707 | 1.00 | 106.03 | O |
| ATOM | 4426 | C | GLU | B | 208 | 60.766 | 24.092 | 32.030 | 1.00 | 120.61 | C |
| ATOM | 4427 | O | GLU | B | 208 | 59.599 | 24.038 | 31.589 | 1.00 | 122.69 | O |
| ATOM | 4428 | N | THR | B | 209 | 61.838 | 23.639 | 31.366 | 1.00 | 117.04 | N |
| ATOM | 4429 | CA | THR | B | 209 | 61.814 | 23.208 | 29.940 | 1.00 | 113.79 | C |
| ATOM | 4430 | CB | THR | B | 209 | 63.214 | 22.883 | 29.402 | 1.00 | 116.01 | C |
| ATOM | 4431 | OG1 | THR | B | 209 | 63.068 | 22.736 | 27.991 | 1.00 | 119.13 | O |
| ATOM | 4432 | CG2 | THR | B | 209 | 63.840 | 21.628 | 29.972 | 1.00 | 116.35 | C |
| ATOM | 4433 | C | THR | B | 209 | 60.849 | 22.028 | 29.763 | 1.00 | 108.69 | C |
| ATOM | 4434 | O | THR | B | 209 | 60.021 | 22.085 | 28.830 | 1.00 | 100.44 | O |
| ATOM | 4435 | N | LYS | B | 210 | 60.944 | 21.003 | 30.614 | 1.00 | 107.92 | N |
| ATOM | 4436 | CA | LYS | B | 210 | 60.090 | 19.793 | 30.491 | 1.00 | 111.42 | C |
| ATOM | 4437 | CB | LYS | B | 210 | 60.632 | 18.613 | 31.308 | 1.00 | 112.76 | C |
| ATOM | 4438 | CG | LYS | B | 210 | 59.811 | 17.330 | 31.187 | 1.00 | 118.27 | C |
| ATOM | 4439 | CD | LYS | B | 210 | 59.381 | 16.966 | 29.751 | 1.00 | 116.07 | C |
| ATOM | 4440 | CE | LYS | B | 210 | 57.960 | 16.443 | 29.630 | 1.00 | 107.26 | C |
| ATOM | 4441 | NZ | LYS | B | 210 | 57.469 | 16.490 | 28.232 | 1.00 | 98.68 | N |
| ATOM | 4442 | C | LYS | B | 210 | 58.655 | 20.156 | 30.892 | 1.00 | 108.61 | C |
| ATOM | 4443 | O | LYS | B | 210 | 57.719 | 19.643 | 30.249 | 1.00 | 116.83 | O |
| ATOM | 4444 | N | ARG | B | 211 | 58.487 | 21.021 | 31.893 | 1.00 | 99.79 | N |
| ATOM | 4445 | CA | ARG | B | 211 | 57.156 | 21.537 | 32.316 | 1.00 | 97.33 | C |
| ATOM | 4446 | CB | ARG | B | 211 | 57.307 | 22.530 | 33.472 | 1.00 | 102.34 | C |
| ATOM | 4447 | CG | ARG | B | 211 | 55.991 | 23.029 | 34.057 | 1.00 | 103.14 | C |
| ATOM | 4448 | CD | ARG | B | 211 | 56.155 | 23.455 | 35.508 | 1.00 | 105.61 | C |
| ATOM | 4449 | NE | ARG | B | 211 | 57.359 | 24.260 | 35.704 | 1.00 | 107.05 | N |
| ATOM | 4450 | CZ | ARG | B | 211 | 57.391 | 25.582 | 35.861 | 1.00 | 107.93 | C |
| ATOM | 4451 | NH1 | ARG | B | 211 | 56.273 | 26.293 | 35.877 | 1.00 | 105.53 | N |
| ATOM | 4452 | NH2 | ARG | B | 211 | 58.559 | 26.186 | 36.006 | 1.00 | 108.31 | N |
| ATOM | 4453 | C | ARG | B | 211 | 56.471 | 22.181 | 31.106 | 1.00 | 87.24 | C |
| ATOM | 4454 | O | ARG | B | 211 | 55.348 | 21.760 | 30.761 | 1.00 | 77.62 | O |
| ATOM | 4455 | N | ASN | B | 212 | 57.142 | 23.146 | 30.473 | 1.00 | 90.53 | N |
| ATOM | 4456 | CA | ASN | B | 212 | 56.627 | 23.861 | 29.274 | 1.00 | 89.98 | C |
| ATOM | 4457 | CB | ASN | B | 212 | 57.629 | 24.889 | 28.737 | 1.00 | 84.55 | C |
| ATOM | 4458 | CG | ASN | B | 212 | 57.530 | 26.221 | 29.455 | 1.00 | 91.93 | C |
| ATOM | 4459 | OD1 | ASN | B | 212 | 58.470 | 26.643 | 30.122 | 1.00 | 94.82 | O |
| ATOM | 4460 | ND2 | ASN | B | 212 | 56.390 | 26.887 | 29.345 | 1.00 | 95.87 | N |
| ATOM | 4461 | C | ASN | B | 212 | 56.210 | 22.819 | 28.232 | 1.00 | 88.27 | C |
| ATOM | 4462 | O | ASN | B | 212 | 55.098 | 22.953 | 27.694 | 1.00 | 93.42 | O |
| ATOM | 4463 | N | GLN | B | 213 | 57.046 | 21.799 | 28.014 | 1.00 | 89.44 | N |
| ATOM | 4464 | CA | GLN | B | 213 | 56.826 | 20.722 | 27.012 | 1.00 | 92.52 | C |
| ATOM | 4465 | CB | GLN | B | 213 | 58.066 | 19.830 | 26.926 | 1.00 | 98.80 | C |
| ATOM | 4466 | CG | GLN | B | 213 | 57.984 | 18.738 | 25.865 | 1.00 | 98.27 | C |
| ATOM | 4467 | CD | GLN | B | 213 | 59.223 | 17.874 | 25.857 | 1.00 | 96.73 | C |
| ATOM | 4468 | OE1 | GLN | B | 213 | 60.122 | 18.030 | 26.680 | 1.00 | 88.59 | O |
| ATOM | 4469 | NE2 | GLN | B | 213 | 59.285 | 16.951 | 24.914 | 1.00 | 99.52 | N |
| ATOM | 4470 | C | GLN | B | 213 | 55.556 | 19.929 | 27.361 | 1.00 | 92.11 | C |
| ATOM | 4471 | O | GLN | B | 213 | 54.754 | 19.683 | 26.435 | 1.00 | 91.50 | O |
| ATOM | 4472 | N | LEU | B | 214 | 55.359 | 19.550 | 28.631 | 1.00 | 83.82 | N |
| ATOM | 4473 | CA | LEU | B | 214 | 54.157 | 18.778 | 29.064 | 1.00 | 79.35 | C |
| ATOM | 4474 | CB | LEU | B | 214 | 54.297 | 18.335 | 30.526 | 1.00 | 82.18 | C |
| ATOM | 4475 | CG | LEU | B | 214 | 53.076 | 17.624 | 31.123 | 1.00 | 74.88 | C |
| ATOM | 4476 | CD1 | LEU | B | 214 | 52.622 | 16.448 | 30.265 | 1.00 | 70.98 | C |
| ATOM | 4477 | CD2 | LEU | B | 214 | 53.360 | 17.161 | 32.535 | 1.00 | 75.36 | C |
| ATOM | 4478 | C | LEU | B | 214 | 52.895 | 19.630 | 28.878 | 1.00 | 77.57 | C |
| ATOM | 4479 | O | LEU | B | 214 | 51.845 | 19.079 | 28.460 | 1.00 | 73.97 | O |
| ATOM | 4480 | N | PHE | B | 215 | 52.972 | 20.927 | 29.181 | 1.00 | 73.95 | N |
| ATOM | 4481 | CA | PHE | B | 215 | 51.801 | 21.830 | 29.079 | 1.00 | 72.98 | C |
| ATOM | 4482 | CB | PHE | B | 215 | 52.135 | 23.226 | 29.601 | 1.00 | 69.01 | C |
| ATOM | 4483 | CG | PHE | B | 215 | 50.957 | 24.158 | 29.523 | 1.00 | 64.32 | C |
| ATOM | 4484 | CD1 | PHE | B | 215 | 49.899 | 24.040 | 30.412 | 1.00 | 65.29 | C |
| ATOM | 4485 | CE1 | PHE | B | 215 | 48.800 | 24.881 | 30.321 | 1.00 | 66.91 | C |
| ATOM | 4486 | CZ | PHE | B | 215 | 48.745 | 25.841 | 29.337 | 1.00 | 66.01 | C |
| ATOM | 4487 | CD2 | PHE | B | 215 | 50.874 | 25.108 | 28.522 | 1.00 | 62.71 | C |
| ATOM | 4488 | CE2 | PHE | B | 215 | 49.780 | 25.955 | 28.439 | 1.00 | 65.03 | C |
| ATOM | 4489 | C | PHE | B | 215 | 51.298 | 21.829 | 27.627 | 1.00 | 77.55 | C |
| ATOM | 4490 | O | PHE | B | 215 | 50.063 | 21.846 | 27.418 | 1.00 | 72.93 | O |
| ATOM | 4491 | N | GLU | B | 216 | 52.223 | 21.757 | 26.661 | 1.00 | 83.49 | N |
| ATOM | 4492 | CA | GLU | B | 216 | 51.917 | 21.729 | 25.201 | 1.00 | 85.59 | C |
| ATOM | 4493 | CB | GLU | B | 216 | 53.193 | 21.851 | 24.360 | 1.00 | 93.10 | C |
| ATOM | 4494 | CG | GLU | B | 216 | 54.137 | 22.975 | 24.787 | 1.00 | 102.23 | C |
| ATOM | 4495 | CD | GLU | B | 216 | 53.631 | 24.405 | 24.640 | 1.00 | 105.12 | C |
| ATOM | 4496 | OE1 | GLU | B | 216 | 53.330 | 24.803 | 23.499 | 1.00 | 101.21 | O |
| ATOM | 4497 | OE2 | GLU | B | 216 | 53.560 | 25.130 | 25.665 | 1.00 | 102.28 | O |
| ATOM | 4498 | C | GLU | B | 216 | 51.150 | 20.436 | 24.883 | 1.00 | 81.74 | C |
| ATOM | 4499 | O | GLU | B | 216 | 50.154 | 20.494 | 24.118 | 1.00 | 67.05 | O |
| ATOM | 4500 | N | ARG | B | 217 | 51.562 | 19.310 | 25.473 | 1.00 | 80.64 | N |
| ATOM | 4501 | CA | ARG | B | 217 | 50.880 | 18.002 | 25.275 | 1.00 | 89.57 | C |
| ATOM | 4502 | CB | ARG | B | 217 | 51.655 | 16.843 | 25.919 | 1.00 | 98.91 | C |
| ATOM | 4503 | CG | ARG | B | 217 | 52.391 | 15.948 | 24.928 | 1.00 | 107.37 | C |
| ATOM | 4504 | CD | ARG | B | 217 | 51.509 | 15.428 | 23.795 | 1.00 | 116.27 | C |
| ATOM | 4505 | NE | ARG | B | 217 | 50.724 | 14.229 | 24.101 | 1.00 | 120.13 | N |
| ATOM | 4506 | CZ | ARG | B | 217 | 49.678 | 13.796 | 23.390 | 1.00 | 114.39 | C |
| ATOM | 4507 | NH1 | ARG | B | 217 | 49.257 | 14.466 | 22.328 | 1.00 | 117.26 | N |
| ATOM | 4508 | NH2 | ARG | B | 217 | 49.039 | 12.696 | 23.749 | 1.00 | 105.05 | N |
| ATOM | 4509 | C | ARG | B | 217 | 49.447 | 18.085 | 25.818 | 1.00 | 83.68 | C |
| ATOM | 4510 | O | ARG | B | 217 | 48.532 | 17.517 | 25.181 | 1.00 | 81.13 | O |
| ATOM | 4511 | N | MET | B | 218 | 49.249 | 18.781 | 26.939 | 1.00 | 78.49 | N |
| ATOM | 4512 | CA | MET | B | 218 | 47.936 | 18.821 | 27.627 | 1.00 | 72.70 | C |
| ATOM | 4513 | CB | MET | B | 218 | 48.089 | 19.279 | 29.077 | 1.00 | 76.47 | C |
| ATOM | 4514 | CG | MET | B | 218 | 48.658 | 18.166 | 29.940 | 1.00 | 85.00 | C |
| ATOM | 4515 | SD | MET | B | 218 | 48.916 | 18.610 | 31.676 | 1.00 | 93.22 | S |
| ATOM | 4516 | CE | MET | B | 218 | 47.247 | 19.062 | 32.148 | 1.00 | 89.82 | C |
| ATOM | 4517 | C | MET | B | 218 | 46.977 | 19.719 | 26.844 | 1.00 | 68.77 | C |
| ATOM | 4518 | O | MET | B | 218 | 45.803 | 19.300 | 26.668 | 1.00 | 65.24 | O |
| ATOM | 4519 | N | LEU | B | 219 | 47.448 | 20.876 | 26.360 | 1.00 | 60.71 | N |
| ATOM | 4520 | CA | LEU | B | 219 | 46.649 | 21.767 | 25.473 | 1.00 | 64.49 | C |
| ATOM | 4521 | CB | LEU | B | 219 | 47.487 | 22.973 | 25.043 | 1.00 | 69.68 | C |
| ATOM | 4522 | CG | LEU | B | 219 | 47.018 | 24.307 | 25.618 | 1.00 | 73.85 | C |
| ATOM | 4523 | CD1 | LEU | B | 219 | 48.157 | 25.310 | 25.621 | 1.00 | 75.65 | C |
| ATOM | 4524 | CD2 | LEU | B | 219 | 45.814 | 24.846 | 24.850 | 1.00 | 75.82 | C |
| ATOM | 4525 | C | LEU | B | 219 | 46.156 | 20.970 | 24.257 | 1.00 | 67.38 | C |
| ATOM | 4526 | O | LEU | B | 219 | 44.959 | 21.113 | 23.883 | 1.00 | 58.36 | O |
| ATOM | 4527 | N | LYS | B | 220 | 47.034 | 20.128 | 23.698 | 1.00 | 70.66 | N |
| ATOM | 4528 | CA | LYS | B | 220 | 46.737 | 19.258 | 22.528 | 1.00 | 69.84 | C |
| ATOM | 4529 | CB | LYS | B | 220 | 47.990 | 18.490 | 22.089 | 1.00 | 82.14 | C |
| ATOM | 4530 | CG | LYS | B | 220 | 47.937 | 17.876 | 20.695 | 1.00 | 86.43 | C |
| ATOM | 4531 | CD | LYS | B | 220 | 49.306 | 17.479 | 20.149 | 1.00 | 92.26 | C |
| ATOM | 4532 | CE | LYS | B | 220 | 49.245 | 16.690 | 18.855 | 1.00 | 96.27 | C |
| ATOM | 4533 | NZ | LYS | B | 220 | 49.187 | 17.572 | 17.665 | 1.00 | 97.25 | N |
| ATOM | 4534 | C | LYS | B | 220 | 45.589 | 18.310 | 22.888 | 1.00 | 62.15 | C |
| ATOM | 4535 | O | LYS | B | 220 | 44.575 | 18.316 | 22.166 | 1.00 | 57.40 | O |
| ATOM | 4536 | N | VAL | B | 221 | 45.726 | 17.521 | 23.957 | 1.00 | 59.40 | N |
| ATOM | 4537 | CA | VAL | B | 221 | 44.715 | 16.463 | 24.270 | 1.00 | 59.33 | C |
| ATOM | 4538 | CB | VAL | B | 221 | 45.193 | 15.419 | 25.311 | 1.00 | 60.58 | C |
| ATOM | 4539 | CG1 | VAL | B | 221 | 45.945 | 16.012 | 26.485 | 1.00 | 67.83 | C |
| ATOM | 4540 | CG2 | VAL | B | 221 | 44.056 | 14.550 | 25.820 | 1.00 | 62.47 | C |
| ATOM | 4541 | C | VAL | B | 221 | 43.386 | 17.146 | 24.629 | 1.00 | 54.40 | C |
| ATOM | 4542 | O | VAL | B | 221 | 42.316 | 16.681 | 24.129 | 1.00 | 48.89 | O |
| ATOM | 4543 | N | HIS | B | 222 | 43.419 | 18.232 | 25.408 | 1.00 | 55.98 | N |
| ATOM | 4544 | CA | HIS | B | 222 | 42.179 | 18.946 | 25.821 | 1.00 | 62.64 | C |
| ATOM | 4545 | CB | HIS | B | 222 | 42.464 | 20.017 | 26.884 | 1.00 | 68.67 | C |
| ATOM | 4546 | CG | HIS | B | 222 | 41.235 | 20.735 | 27.338 | 1.00 | 65.74 | C |
| ATOM | 4547 | ND1 | HIS | B | 222 | 40.447 | 20.268 | 28.374 | 1.00 | 64.15 | N |
| ATOM | 4548 | CE1 | HIS | B | 222 | 39.436 | 21.090 | 28.555 | 1.00 | 62.70 | C |
| ATOM | 4549 | NE2 | HIS | B | 222 | 39.536 | 22.076 | 27.656 | 1.00 | 68.35 | N |
| ATOM | 4550 | CD2 | HIS | B | 222 | 40.645 | 21.862 | 26.885 | 1.00 | 62.38 | C |
| ATOM | 4551 | C | HIS | B | 222 | 41.461 | 19.517 | 24.588 | 1.00 | 60.46 | C |
| ATOM | 4552 | O | HIS | B | 222 | 40.238 | 19.346 | 24.511 | 1.00 | 57.11 | O |
| ATOM | 4553 | N | SER | B | 223 | 42.188 | 20.165 | 23.669 | 1.00 | 63.24 | N |
| ATOM | 4554 | CA | SER | B | 223 | 41.665 | 20.690 | 22.374 | 1.00 | 66.81 | C |
| ATOM | 4555 | CB | SER | B | 223 | 42.760 | 21.321 | 21.560 | 1.00 | 68.31 | C |
| ATOM | 4556 | OG | SER | B | 223 | 43.665 | 22.003 | 22.414 | 1.00 | 78.90 | O |
| ATOM | 4557 | C | SER | B | 223 | 40.979 | 19.574 | 21.576 | 1.00 | 63.08 | C |
| ATOM | 4558 | O | SER | B | 223 | 39.876 | 19.815 | 21.058 | 1.00 | 64.55 | O |
| ATOM | 4559 | N | ARG | B | 224 | 41.599 | 18.394 | 21.489 | 1.00 | 58.08 | N |
| ATOM | 4560 | CA | ARG | B | 224 | 41.046 | 17.229 | 20.745 | 1.00 | 62.19 | C |
| ATOM | 4561 | CB | ARG | B | 224 | 42.148 | 16.188 | 20.527 | 1.00 | 68.15 | C |
| ATOM | 4562 | CG | ARG | B | 224 | 43.171 | 16.575 | 19.467 | 1.00 | 71.87 | C |
| ATOM | 4563 | CD | ARG | B | 224 | 44.390 | 15.685 | 19.566 | 1.00 | 84.65 | C |
| ATOM | 4564 | NE | ARG | B | 224 | 45.011 | 15.406 | 18.279 | 1.00 | 91.47 | N |
| ATOM | 4565 | CZ | ARG | B | 224 | 45.798 | 16.242 | 17.615 | 1.00 | 92.63 | C |
| ATOM | 4566 | NH1 | ARG | B | 224 | 46.064 | 17.445 | 18.098 | 1.00 | 96.81 | N |
| ATOM | 4567 | NH2 | ARG | B | 224 | 46.317 | 15.869 | 16.459 | 1.00 | 96.85 | N |
| ATOM | 4568 | C | ARG | B | 224 | 39.813 | 16.638 | 21.459 | 1.00 | 59.71 | C |
| ATOM | 4569 | O | ARG | B | 224 | 39.122 | 15.828 | 20.840 | 1.00 | 66.79 | O |
| ATOM | 4570 | N | GLY | B | 225 | 39.513 | 17.038 | 22.698 | 1.00 | 59.00 | N |
| ATOM | 4571 | CA | GLY | B | 225 | 38.347 | 16.544 | 23.461 | 1.00 | 51.91 | C |
| ATOM | 4572 | C | GLY | B | 225 | 38.717 | 15.362 | 24.351 | 1.00 | 45.00 | C |
| ATOM | 4573 | O | GLY | B | 225 | 37.834 | 14.539 | 24.652 | 1.00 | 38.50 | O |
| ATOM | 4574 | N | GLY | B | 226 | 39.990 | 15.294 | 24.752 | 1.00 | 45.22 | N |
| ATOM | 4575 | CA | GLY | B | 226 | 40.512 | 14.339 | 25.745 | 1.00 | 49.73 | C |
| ATOM | 4576 | C | GLY | B | 226 | 40.054 | 14.663 | 27.171 | 1.00 | 51.69 | C |
| ATOM | 4577 | O | GLY | B | 226 | 39.441 | 15.721 | 27.411 | 1.00 | 48.60 | O |
| ATOM | 4578 | N | LEU | B | 227 | 40.292 | 13.730 | 28.084 | 1.00 | 48.22 | N |
| ATOM | 4579 | CA | LEU | B | 227 | 40.059 | 13.893 | 29.529 | 1.00 | 44.45 | C |
| ATOM | 4580 | CB | LEU | B | 227 | 39.266 | 12.686 | 30.019 | 1.00 | 42.01 | C |
| ATOM | 4581 | CG | LEU | B | 227 | 37.842 | 12.623 | 29.480 | 1.00 | 43.78 | C |
| ATOM | 4582 | CD1 | LEU | B | 227 | 37.163 | 11.320 | 29.851 | 1.00 | 43.13 | C |
| ATOM | 4583 | CD2 | LEU | B | 227 | 37.025 | 13.797 | 29.981 | 1.00 | 45.70 | C |
| ATOM | 4584 | C | LEU | B | 227 | 41.423 | 14.019 | 30.202 | 1.00 | 47.29 | C |
| ATOM | 4585 | O | LEU | B | 227 | 42.328 | 13.238 | 29.866 | 1.00 | 53.17 | O |
| ATOM | 4586 | N | ILE | B | 228 | 41.578 | 15.018 | 31.066 | 1.00 | 49.77 | N |
| ATOM | 4587 | CA | ILE | B | 228 | 42.755 | 15.130 | 31.966 | 1.00 | 51.36 | C |
| ATOM | 4588 | CB | ILE | B | 228 | 43.624 | 16.338 | 31.581 | 1.00 | 52.61 | C |
| ATOM | 4589 | CG1 | ILE | B | 228 | 44.022 | 16.273 | 30.102 | 1.00 | 55.79 | C |
| ATOM | 4590 | CG2 | ILE | B | 228 | 44.844 | 16.428 | 32.486 | 1.00 | 52.69 | C |
| ATOM | 4591 | CD1 | ILE | B | 228 | 44.153 | 17.617 | 29.423 | 1.00 | 56.01 | C |
| ATOM | 4592 | C | ILE | B | 228 | 42.232 | 15.170 | 33.404 | 1.00 | 54.35 | C |
| ATOM | 4593 | O | ILE | B | 228 | 41.112 | 15.692 | 33.616 | 1.00 | 52.11 | O |
| ATOM | 4594 | N | SER | B | 229 | 42.974 | 14.531 | 34.316 | 1.00 | 57.81 | N |
| ATOM | 4595 | CA | SER | B | 229 | 42.718 | 14.479 | 35.781 | 1.00 | 54.44 | C |
| ATOM | 4596 | CB | SER | B | 229 | 42.065 | 13.187 | 36.227 | 1.00 | 56.72 | C |
| ATOM | 4597 | OG | SER | B | 229 | 40.646 | 13.332 | 36.372 | 1.00 | 61.87 | O |
| ATOM | 4598 | C | SER | B | 229 | 44.050 | 14.696 | 36.491 | 1.00 | 53.71 | C |
| ATOM | 4599 | O | SER | B | 229 | 45.055 | 14.157 | 36.022 | 1.00 | 54.66 | O |
| ATOM | 4600 | N | ALA | B | 230 | 44.053 | 15.515 | 37.539 | 1.00 | 51.25 | N |
| ATOM | 4601 | CA | ALA | B | 230 | 45.216 | 15.730 | 38.421 | 1.00 | 52.08 | C |
| ATOM | 4602 | CB | ALA | B | 230 | 45.636 | 17.175 | 38.419 | 1.00 | 53.20 | C |
| ATOM | 4603 | C | ALA | B | 230 | 44.819 | 15.247 | 39.813 | 1.00 | 53.83 | C |
| ATOM | 4604 | O | ALA | B | 230 | 43.648 | 15.458 | 40.231 | 1.00 | 50.12 | O |
| ATOM | 4605 | N | SER | B | 231 | 45.758 | 14.586 | 40.476 | 1.00 | 54.56 | N |
| ATOM | 4606 | CA | SER | B | 231 | 45.522 | 13.840 | 41.731 | 1.00 | 60.81 | C |
| ATOM | 4607 | CB | SER | B | 231 | 45.276 | 12.368 | 41.464 | 1.00 | 61.62 | C |
| ATOM | 4608 | OG | SER | B | 231 | 46.375 | 11.773 | 40.781 | 1.00 | 62.93 | O |
| ATOM | 4609 | C | SER | B | 231 | 46.708 | 14.061 | 42.663 | 1.00 | 56.63 | C |
| ATOM | 4610 | O | SER | B | 231 | 47.811 | 14.375 | 42.174 | 1.00 | 49.92 | O |
| ATOM | 4611 | N | ILE | B | 232 | 46.449 | 13.925 | 43.956 | 1.00 | 57.87 | N |
| ATOM | 4612 | CA | ILE | B | 232 | 47.481 | 13.958 | 45.021 | 1.00 | 63.78 | C |
| ATOM | 4613 | CB | ILE | B | 232 | 47.102 | 15.022 | 46.068 | 1.00 | 71.92 | C |
| ATOM | 4614 | CG1 | ILE | B | 232 | 46.881 | 16.377 | 45.387 | 1.00 | 74.45 | C |
| ATOM | 4615 | CG2 | ILE | B | 232 | 48.145 | 15.094 | 47.173 | 1.00 | 75.89 | C |
| ATOM | 4616 | CD1 | ILE | B | 232 | 46.480 | 17.494 | 46.315 | 1.00 | 75.26 | C |
| ATOM | 4617 | C | ILE | B | 232 | 47.581 | 12.535 | 45.576 | 1.00 | 59.69 | C |
| ATOM | 4618 | O | ILE | B | 232 | 46.576 | 12.051 | 46.127 | 1.00 | 58.45 | O |
| ATOM | 4619 | N | LYS | B | 233 | 48.734 | 11.898 | 45.360 | 1.00 | 62.83 | N |
| ATOM | 4620 | CA | LYS | B | 233 | 49.131 | 10.557 | 45.869 | 1.00 | 70.60 | C |
| ATOM | 4621 | CB | LYS | B | 233 | 50.604 | 10.328 | 45.500 | 1.00 | 80.04 | C |
| ATOM | 4622 | CG | LYS | B | 233 | 51.132 | 8.897 | 45.501 | 1.00 | 83.86 | C |
| ATOM | 4623 | CD | LYS | B | 233 | 52.421 | 8.769 | 44.683 | 1.00 | 89.57 | C |
| ATOM | 4624 | CE | LYS | B | 233 | 53.461 | 7.833 | 45.267 | 1.00 | 94.62 | C |
| ATOM | 4625 | NZ | LYS | B | 233 | 53.181 | 6.414 | 44.940 | 1.00 | 92.56 | N |
| ATOM | 4626 | C | LYS | B | 233 | 48.928 | 10.510 | 47.387 | 1.00 | 74.11 | C |
| ATOM | 4627 | O | LYS | B | 233 | 49.638 | 11.259 | 48.085 | 1.00 | 81.79 | O |
| ATOM | 4628 | N | ALA | B | 234 | 47.980 | 9.699 | 47.869 | 1.00 | 73.87 | N |
| ATOM | 4629 | CA | ALA | B | 234 | 47.859 | 9.295 | 49.292 | 1.00 | 87.71 | C |
| ATOM | 4630 | CB | ALA | B | 234 | 46.423 | 8.933 | 49.621 | 1.00 | 88.08 | C |
| ATOM | 4631 | C | ALA | B | 234 | 48.835 | 8.134 | 49.569 | 1.00 | 86.06 | C |
| ATOM | 4632 | O | ALA | B | 234 | 48.500 | 6.985 | 49.220 | 1.00 | 79.71 | O |
| ATOM | 4633 | N | VAL | B | 235 | 49.987 | 8.437 | 50.189 | 1.00 | 91.44 | N |
| ATOM | 4634 | CA | VAL | B | 235 | 51.179 | 7.541 | 50.338 | 1.00 | 98.00 | C |
| ATOM | 4635 | CB | VAL | B | 235 | 52.455 | 8.347 | 50.683 | 1.00 | 108.21 | C |
| ATOM | 4636 | CG1 | VAL | B | 235 | 53.726 | 7.509 | 50.595 | 1.00 | 106.49 | C |
| ATOM | 4637 | CG2 | VAL | B | 235 | 52.602 | 9.595 | 49.820 | 1.00 | 110.73 | C |
| ATOM | 4638 | C | VAL | B | 235 | 50.867 | 6.477 | 51.403 | 1.00 | 104.88 | C |
| ATOM | 4639 | O | VAL | B | 235 | 50.715 | 5.287 | 51.023 | 1.00 | 90.97 | O |
| ATOM | 4640 | N | THR | B | 236 | 50.744 | 6.892 | 52.675 | 1.00 | 110.53 | N |
| ATOM | 4641 | CA | THR | B | 236 | 50.490 | 6.015 | 53.851 | 1.00 | 102.04 | C |
| ATOM | 4642 | CB | THR | B | 236 | 51.002 | 6.606 | 55.173 | 1.00 | 108.50 | C |
| ATOM | 4643 | OG1 | THR | B | 236 | 49.991 | 7.480 | 55.681 | 1.00 | 110.60 | O |
| ATOM | 4644 | CG2 | THR | B | 236 | 52.327 | 7.330 | 55.045 | 1.00 | 108.40 | C |
| ATOM | 4645 | C | THR | B | 236 | 48.987 | 5.794 | 53.989 | 1.00 | 95.87 | C |
| ATOM | 4646 | O | THR | B | 236 | 48.228 | 6.547 | 53.371 | 1.00 | 90.44 | O |
| ATOM | 4647 | N | ALA | B | 237 | 48.594 | 4.808 | 54.796 | 1.00 | 105.30 | N |
| ATOM | 4648 | CA | ALA | B | 237 | 47.187 | 4.478 | 55.117 | 1.00 | 107.08 | C |
| ATOM | 4649 | CB | ALA | B | 237 | 47.150 | 3.381 | 56.156 | 1.00 | 108.68 | C |
| ATOM | 4650 | C | ALA | B | 237 | 46.459 | 5.745 | 55.592 | 1.00 | 104.64 | C |
| ATOM | 4651 | O | ALA | B | 237 | 45.378 | 6.040 | 55.050 | 1.00 | 94.81 | O |
| ATOM | 4652 | N | ALA | B | 238 | 47.062 | 6.487 | 56.531 | 1.00 | 102.29 | N |
| ATOM | 4653 | CA | ALA | B | 238 | 46.449 | 7.626 | 57.259 | 1.00 | 98.36 | C |
| ATOM | 4654 | CB | ALA | B | 238 | 47.164 | 7.851 | 58.570 | 1.00 | 99.28 | C |
| ATOM | 4655 | C | ALA | B | 238 | 46.483 | 8.901 | 56.412 | 1.00 | 98.37 | C |
| ATOM | 4656 | O | ALA | B | 238 | 46.070 | 9.958 | 56.942 | 1.00 | 93.56 | O |
| ATOM | 4657 | N | ASP | B | 239 | 46.972 | 8.821 | 55.168 | 1.00 | 97.56 | N |
| ATOM | 4658 | CA | ASP | B | 239 | 47.028 | 9.977 | 54.232 | 1.00 | 94.68 | C |
| ATOM | 4659 | CB | ASP | B | 239 | 48.188 | 9.839 | 53.242 | 1.00 | 93.30 | C |
| ATOM | 4660 | CG | ASP | B | 239 | 49.464 | 10.518 | 53.715 | 1.00 | 95.21 | C |
| ATOM | 4661 | OD1 | ASP | B | 239 | 49.372 | 11.681 | 54.200 | 1.00 | 85.34 | O |
| ATOM | 4662 | OD2 | ASP | B | 239 | 50.542 | 9.884 | 53.596 | 1.00 | 92.33 | O |
| ATOM | 4663 | C | ASP | B | 239 | 45.667 | 10.167 | 53.544 | 1.00 | 87.43 | C |
| ATOM | 4664 | O | ASP | B | 239 | 45.344 | 11.321 | 53.202 | 1.00 | 77.61 | O |
| ATOM | 4665 | N | MET | B | 240 | 44.879 | 9.102 | 53.379 | 1.00 | 81.75 | N |
| ATOM | 4666 | CA | MET | B | 240 | 43.541 | 9.168 | 52.726 | 1.00 | 81.32 | C |
| ATOM | 4667 | CB | MET | B | 240 | 42.725 | 7.886 | 52.938 | 1.00 | 83.23 | C |
| ATOM | 4668 | CG | MET | B | 240 | 41.717 | 7.648 | 51.832 | 1.00 | 86.39 | C |
| ATOM | 4669 | SD | MET | B | 240 | 42.506 | 7.649 | 50.190 | 1.00 | 91.95 | S |
| ATOM | 4670 | CE | MET | B | 240 | 41.103 | 8.127 | 49.180 | 1.00 | 95.86 | C |
| ATOM | 4671 | C | MET | B | 240 | 42.746 | 10.364 | 53.271 | 1.00 | 72.61 | C |
| ATOM | 4672 | O | MET | B | 240 | 42.479 | 10.396 | 54.485 | 1.00 | 65.69 | O |
| ATOM | 4673 | N | GLU | B | 241 | 42.407 | 11.308 | 52.384 | 1.00 | 70.15 | N |
| ATOM | 4674 | CA | GLU | B | 241 | 41.518 | 12.479 | 52.622 | 1.00 | 62.30 | C |
| ATOM | 4675 | CB | GLU | B | 241 | 40.122 | 12.027 | 53.049 | 1.00 | 57.44 | C |
| ATOM | 4676 | CG | GLU | B | 241 | 39.417 | 11.291 | 51.925 | 1.00 | 63.93 | C |
| ATOM | 4677 | CD | GLU | B | 241 | 37.913 | 11.161 | 52.060 | 1.00 | 67.43 | C |
| ATOM | 4678 | OE1 | GLU | B | 241 | 37.489 | 10.464 | 53.002 | 1.00 | 67.20 | O |
| ATOM | 4679 | OE2 | GLU | B | 241 | 37.168 | 11.751 | 51.211 | 1.00 | 62.37 | O |
| ATOM | 4680 | C | GLU | B | 241 | 42.163 | 13.441 | 53.620 | 1.00 | 60.54 | C |
| ATOM | 4681 | O | GLU | B | 241 | 41.425 | 14.122 | 54.326 | 1.00 | 62.43 | O |
| ATOM | 4682 | N | ALA | B | 242 | 43.492 | 13.529 | 53.637 | 1.00 | 63.53 | N |
| ATOM | 4683 | CA | ALA | B | 242 | 44.230 | 14.580 | 54.373 | 1.00 | 70.12 | C |
| ATOM | 4684 | CB | ALA | B | 242 | 45.661 | 14.163 | 54.635 | 1.00 | 68.63 | C |
| ATOM | 4685 | C | ALA | B | 242 | 44.170 | 15.863 | 53.540 | 1.00 | 67.55 | C |
| ATOM | 4686 | O | ALA | B | 242 | 44.478 | 15.786 | 52.340 | 1.00 | 65.98 | O |
| ATOM | 4687 | N | ARG | B | 243 | 43.748 | 16.967 | 54.164 | 1.00 | 68.99 | N |
| ATOM | 4688 | CA | ARG | B | 243 | 43.766 | 18.350 | 53.613 | 1.00 | 66.71 | C |
| ATOM | 4689 | CB | ARG | B | 243 | 42.795 | 19.229 | 54.414 | 1.00 | 65.20 | C |
| ATOM | 4690 | CG | ARG | B | 243 | 41.486 | 19.582 | 53.711 | 1.00 | 73.82 | C |
| ATOM | 4691 | CD | ARG | B | 243 | 41.121 | 21.057 | 53.861 | 1.00 | 81.35 | C |
| ATOM | 4692 | NE | ARG | B | 243 | 42.345 | 21.863 | 53.944 | 1.00 | 90.42 | N |
| ATOM | 4693 | CZ | ARG | B | 243 | 42.442 | 23.133 | 54.337 | 1.00 | 85.31 | C |
| ATOM | 4694 | NH1 | ARG | B | 243 | 41.369 | 23.813 | 54.707 | 1.00 | 82.45 | N |
| ATOM | 4695 | NH2 | ARG | B | 243 | 43.634 | 23.711 | 54.364 | 1.00 | 84.36 | N |
| ATOM | 4696 | C | ARG | B | 243 | 45.213 | 18.872 | 53.675 | 1.00 | 67.86 | C |
| ATOM | 4697 | O | ARG | B | 243 | 45.876 | 18.627 | 54.696 | 1.00 | 78.27 | O |
| ATOM | 4698 | N | LEU | B | 244 | 45.704 | 19.519 | 52.613 | 1.00 | 68.75 | N |
| ATOM | 4699 | CA | LEU | B | 244 | 46.984 | 20.277 | 52.608 | 1.00 | 67.42 | C |
| ATOM | 4700 | CB | LEU | B | 244 | 47.594 | 20.250 | 51.204 | 1.00 | 74.13 | C |
| ATOM | 4701 | CG | LEU | B | 244 | 48.022 | 18.878 | 50.692 | 1.00 | 71.00 | C |
| ATOM | 4702 | CD1 | LEU | B | 244 | 48.633 | 18.987 | 49.302 | 1.00 | 68.44 | C |
| ATOM | 4703 | CD2 | LEU | B | 244 | 49.006 | 18.235 | 51.652 | 1.00 | 72.27 | C |
| ATOM | 4704 | C | LEU | B | 244 | 46.686 | 21.714 | 53.029 | 1.00 | 67.98 | C |
| ATOM | 4705 | O | LEU | B | 244 | 45.483 | 22.085 | 53.057 | 1.00 | 64.56 | O |
| ATOM | 4706 | N | ALA | B | 245 | 47.728 | 22.496 | 53.327 | 1.00 | 70.68 | N |
| ATOM | 4707 | CA | ALA | B | 245 | 47.587 | 23.932 | 53.679 | 1.00 | 72.24 | C |
| ATOM | 4708 | CB | ALA | B | 245 | 48.941 | 24.566 | 53.882 | 1.00 | 69.41 | C |
| ATOM | 4709 | C | ALA | B | 245 | 46.786 | 24.651 | 52.581 | 1.00 | 71.17 | C |
| ATOM | 4710 | O | ALA | B | 245 | 45.982 | 25.529 | 52.921 | 1.00 | 63.10 | O |
| ATOM | 4711 | N | CYS | B | 246 | 46.970 | 24.270 | 51.312 | 1.00 | 69.33 | N |
| ATOM | 4712 | CA | CYS | B | 246 | 46.365 | 24.958 | 50.144 | 1.00 | 65.09 | C |
| ATOM | 4713 | CB | CYS | B | 246 | 47.177 | 24.717 | 48.886 | 1.00 | 62.36 | C |
| ATOM | 4714 | SG | CYS | B | 246 | 47.002 | 23.030 | 48.263 | 1.00 | 63.22 | S |
| ATOM | 4715 | C | CYS | B | 246 | 44.925 | 24.492 | 49.916 | 1.00 | 64.90 | C |
| ATOM | 4716 | O | CYS | B | 246 | 44.239 | 25.087 | 49.064 | 1.00 | 73.85 | O |
| ATOM | 4717 | N | GLY | B | 247 | 44.460 | 23.485 | 50.649 | 1.00 | 64.15 | N |
| ATOM | 4718 | CA | GLY | B | 247 | 43.030 | 23.111 | 50.648 | 1.00 | 62.26 | C |
| ATOM | 4719 | C | GLY | B | 247 | 42.745 | 21.876 | 49.811 | 1.00 | 59.74 | C |
| ATOM | 4720 | O | GLY | B | 247 | 41.678 | 21.264 | 50.022 | 1.00 | 63.64 | O |
| ATOM | 4721 | N | LEU | B | 248 | 43.663 | 21.504 | 48.915 | 1.00 | 58.50 | N |
| ATOM | 4722 | CA | LEU | B | 248 | 43.546 | 20.283 | 48.068 | 1.00 | 61.12 | C |
| ATOM | 4723 | CB | LEU | B | 248 | 44.539 | 20.352 | 46.902 | 1.00 | 59.30 | C |
| ATOM | 4724 | CG | LEU | B | 248 | 44.380 | 21.570 | 45.991 | 1.00 | 56.00 | C |
| ATOM | 4725 | CD1 | LEU | B | 248 | 45.547 | 21.686 | 45.020 | 1.00 | 53.28 | C |
| ATOM | 4726 | CD2 | LEU | B | 248 | 43.042 | 21.523 | 45.260 | 1.00 | 53.95 | C |
| ATOM | 4727 | C | LEU | B | 248 | 43.797 | 19.043 | 48.927 | 1.00 | 63.27 | C |
| ATOM | 4728 | O | LEU | B | 248 | 44.573 | 19.127 | 49.901 | 1.00 | 72.55 | O |
| ATOM | 4729 | N | VAL | B | 249 | 43.209 | 17.924 | 48.516 | 1.00 | 62.32 | N |
| ATOM | 4730 | CA | VAL | B | 249 | 42.967 | 16.715 | 49.350 | 1.00 | 58.80 | C |
| ATOM | 4731 | CB | VAL | B | 249 | 41.458 | 16.455 | 49.403 | 1.00 | 59.66 | C |
| ATOM | 4732 | CG1 | VAL | B | 249 | 41.135 | 15.153 | 50.120 | 1.00 | 69.62 | C |
| ATOM | 4733 | CG2 | VAL | B | 249 | 40.744 | 17.630 | 50.040 | 1.00 | 57.24 | C |
| ATOM | 4734 | C | VAL | B | 249 | 43.732 | 15.508 | 48.793 | 1.00 | 57.57 | C |
| ATOM | 4735 | O | VAL | B | 249 | 43.532 | 15.154 | 47.607 | 1.00 | 52.93 | O |
| ATOM | 4736 | N | LYS | B | 250 | 44.548 | 14.866 | 49.638 | 1.00 | 65.50 | N |
| ATOM | 4737 | CA | LYS | B | 250 | 45.324 | 13.647 | 49.268 | 1.00 | 68.33 | C |
| ATOM | 4738 | CB | LYS | B | 250 | 46.354 | 13.291 | 50.345 | 1.00 | 68.15 | C |
| ATOM | 4739 | CG | LYS | B | 250 | 47.422 | 14.337 | 50.637 | 1.00 | 73.64 | C |
| ATOM | 4740 | CD | LYS | B | 250 | 48.715 | 13.710 | 51.124 | 1.00 | 81.89 | C |
| ATOM | 4741 | CE | LYS | B | 250 | 49.819 | 14.703 | 51.407 | 1.00 | 89.18 | C |
| ATOM | 4742 | NZ | LYS | B | 250 | 50.959 | 14.048 | 52.093 | 1.00 | 98.84 | N |
| ATOM | 4743 | C | LYS | B | 250 | 44.331 | 12.497 | 49.023 | 1.00 | 64.03 | C |
| ATOM | 4744 | O | LYS | B | 250 | 43.399 | 12.338 | 49.875 | 1.00 | 57.81 | O |
| ATOM | 4745 | N | GLY | B | 251 | 44.493 | 11.773 | 47.896 | 1.00 | 57.94 | N |
| ATOM | 4746 | CA | GLY | B | 251 | 43.643 | 10.630 | 47.486 | 1.00 | 58.20 | C |
| ATOM | 4747 | C | GLY | B | 251 | 42.395 | 11.066 | 46.712 | 1.00 | 57.91 | C |
| ATOM | 4748 | O | GLY | B | 251 | 41.544 | 10.215 | 46.354 | 1.00 | 57.98 | O |
| ATOM | 4749 | N | HIS | B | 252 | 42.255 | 12.361 | 46.468 | 1.00 | 56.23 | N |
| ATOM | 4750 | CA | HIS | B | 252 | 41.236 | 12.919 | 45.554 | 1.00 | 51.92 | C |
| ATOM | 4751 | CB | HIS | B | 252 | 40.583 | 14.151 | 46.202 | 1.00 | 54.86 | C |
| ATOM | 4752 | CG | HIS | B | 252 | 39.595 | 13.816 | 47.272 | 1.00 | 55.01 | C |
| ATOM | 4753 | ND1 | HIS | B | 252 | 38.733 | 14.750 | 47.796 | 1.00 | 54.16 | N |
| ATOM | 4754 | CE1 | HIS | B | 252 | 37.939 | 14.183 | 48.676 | 1.00 | 50.46 | C |
| ATOM | 4755 | NE2 | HIS | B | 252 | 38.249 | 12.889 | 48.736 | 1.00 | 55.24 | N |
| ATOM | 4756 | CD2 | HIS | B | 252 | 39.274 | 12.645 | 47.864 | 1.00 | 56.64 | C |
| ATOM | 4757 | C | HIS | B | 252 | 41.931 | 13.182 | 44.220 | 1.00 | 51.44 | C |
| ATOM | 4758 | O | HIS | B | 252 | 43.165 | 13.419 | 44.196 | 1.00 | 48.89 | O |
| ATOM | 4759 | N | ALA | B | 253 | 41.181 | 13.094 | 43.130 | 1.00 | 56.76 | N |
| ATOM | 4760 | CA | ALA | B | 253 | 41.637 | 13.543 | 41.798 | 1.00 | 57.09 | C |
| ATOM | 4761 | CB | ALA | B | 253 | 41.699 | 12.394 | 40.828 | 1.00 | 58.01 | C |
| ATOM | 4762 | C | ALA | B | 253 | 40.677 | 14.628 | 41.334 | 1.00 | 54.21 | C |
| ATOM | 4763 | O | ALA | B | 253 | 39.481 | 14.545 | 41.699 | 1.00 | 54.99 | O |
| ATOM | 4764 | N | TYR | B | 254 | 41.192 | 15.600 | 40.589 | 1.00 | 47.91 | N |
| ATOM | 4765 | CA | TYR | B | 254 | 40.413 | 16.765 | 40.094 | 1.00 | 51.27 | C |
| ATOM | 4766 | CB | TYR | B | 254 | 41.016 | 18.046 | 40.672 | 1.00 | 51.33 | C |
| ATOM | 4767 | CG | TYR | B | 254 | 41.008 | 18.062 | 42.179 | 1.00 | 52.14 | C |
| ATOM | 4768 | CD1 | TYR | B | 254 | 42.065 | 17.546 | 42.905 | 1.00 | 51.70 | C |
| ATOM | 4769 | CE1 | TYR | B | 254 | 42.049 | 17.542 | 44.287 | 1.00 | 52.85 | C |
| ATOM | 4770 | CZ | TYR | B | 254 | 40.957 | 18.056 | 44.962 | 1.00 | 54.60 | C |
| ATOM | 4771 | OH | TYR | B | 254 | 40.926 | 18.057 | 46.322 | 1.00 | 56.03 | O |
| ATOM | 4772 | CE2 | TYR | B | 254 | 39.883 | 18.564 | 44.255 | 1.00 | 52.44 | C |
| ATOM | 4773 | CD2 | TYR | B | 254 | 39.925 | 18.571 | 42.873 | 1.00 | 51.95 | C |
| ATOM | 4774 | C | TYR | B | 254 | 40.393 | 16.734 | 38.563 | 1.00 | 45.54 | C |
| ATOM | 4775 | O | TYR | B | 254 | 41.448 | 16.538 | 37.949 | 1.00 | 41.64 | O |
| ATOM | 4776 | N | ALA | B | 255 | 39.221 | 16.911 | 37.968 | 1.00 | 40.85 | N |
| ATOM | 4777 | CA | ALA | B | 255 | 39.037 | 16.924 | 36.502 | 1.00 | 43.32 | C |
| ATOM | 4778 | CB | ALA | B | 255 | 37.593 | 16.625 | 36.169 | 1.00 | 41.43 | C |
| ATOM | 4779 | C | ALA | B | 255 | 39.501 | 18.285 | 35.955 | 1.00 | 46.77 | C |
| ATOM | 4780 | O | ALA | B | 255 | 39.076 | 19.334 | 36.487 | 1.00 | 43.48 | O |
| ATOM | 4781 | N | VAL | B | 256 | 40.351 | 18.281 | 34.929 | 1.00 | 46.84 | N |
| ATOM | 4782 | CA | VAL | B | 256 | 40.682 | 19.519 | 34.172 | 1.00 | 47.41 | C |
| ATOM | 4783 | CB | VAL | B | 256 | 41.952 | 19.346 | 33.334 | 1.00 | 52.63 | C |
| ATOM | 4784 | CG1 | VAL | B | 256 | 42.100 | 20.483 | 32.340 | 1.00 | 53.44 | C |
| ATOM | 4785 | CG2 | VAL | B | 256 | 43.183 | 19.233 | 34.226 | 1.00 | 53.13 | C |
| ATOM | 4786 | C | VAL | B | 256 | 39.486 | 19.868 | 33.296 | 1.00 | 48.48 | C |
| ATOM | 4787 | O | VAL | B | 256 | 39.026 | 18.983 | 32.570 | 1.00 | 51.19 | O |
| ATOM | 4788 | N | THR | B | 257 | 38.972 | 21.093 | 33.392 | 1.00 | 46.00 | N |
| ATOM | 4789 | CA | THR | B | 257 | 37.939 | 21.589 | 32.452 | 1.00 | 46.15 | C |
| ATOM | 4790 | CB | THR | B | 257 | 36.628 | 21.953 | 33.161 | 1.00 | 45.27 | C |
| ATOM | 4791 | OG1 | THR | B | 257 | 36.885 | 22.968 | 34.126 | 1.00 | 43.39 | O |
| ATOM | 4792 | CG2 | THR | B | 257 | 35.971 | 20.751 | 33.801 | 1.00 | 45.70 | C |
| ATOM | 4793 | C | THR | B | 257 | 38.493 | 22.741 | 31.601 | 1.00 | 45.27 | C |
| ATOM | 4794 | O | THR | B | 257 | 37.731 | 23.236 | 30.775 | 1.00 | 43.55 | O |
| ATOM | 4795 | N | ASP | B | 258 | 39.756 | 23.146 | 31.764 | 1.00 | 44.30 | N |
| ATOM | 4796 | CA | ASP | B | 258 | 40.395 | 24.027 | 30.765 | 1.00 | 44.94 | C |
| ATOM | 4797 | CB | ASP | B | 258 | 39.832 | 25.446 | 30.834 | 1.00 | 51.58 | C |
| ATOM | 4798 | CG | ASP | B | 258 | 40.032 | 26.213 | 29.530 | 1.00 | 57.46 | C |
| ATOM | 4799 | OD1 | ASP | B | 258 | 40.565 | 25.606 | 28.561 | 1.00 | 63.12 | O |
| ATOM | 4800 | OD2 | ASP | B | 258 | 39.665 | 27.407 | 29.485 | 1.00 | 55.63 | O |
| ATOM | 4801 | C | ASP | B | 258 | 41.925 | 23.974 | 30.864 | 1.00 | 45.87 | C |
| ATOM | 4802 | O | ASP | B | 258 | 42.467 | 23.835 | 31.976 | 1.00 | 45.42 | O |
| ATOM | 4803 | N | VAL | B | 259 | 42.579 | 24.022 | 29.695 | 1.00 | 47.14 | N |
| ATOM | 4804 | CA | VAL | B | 259 | 44.044 | 24.253 | 29.516 | 1.00 | 51.93 | C |
| ATOM | 4805 | CB | VAL | B | 259 | 44.788 | 23.012 | 29.003 | 1.00 | 49.35 | C |
| ATOM | 4806 | CG1 | VAL | B | 259 | 46.289 | 23.170 | 29.133 | 1.00 | 46.12 | C |
| ATOM | 4807 | CG2 | VAL | B | 259 | 44.327 | 21.749 | 29.704 | 1.00 | 54.40 | C |
| ATOM | 4808 | C | VAL | B | 259 | 44.167 | 25.395 | 28.514 | 1.00 | 60.46 | C |
| ATOM | 4809 | O | VAL | B | 259 | 43.452 | 25.343 | 27.480 | 1.00 | 61.88 | O |
| ATOM | 4810 | N | ARG | B | 260 | 44.991 | 26.400 | 28.822 | 1.00 | 61.06 | N |
| ATOM | 4811 | CA | ARG | B | 260 | 44.881 | 27.719 | 28.151 | 1.00 | 63.81 | C |
| ATOM | 4812 | CB | ARG | B | 260 | 43.602 | 28.431 | 28.607 | 1.00 | 62.27 | C |
| ATOM | 4813 | CG | ARG | B | 260 | 43.249 | 29.651 | 27.775 | 1.00 | 64.25 | C |
| ATOM | 4814 | CD | ARG | B | 260 | 42.020 | 30.341 | 28.312 | 1.00 | 64.87 | C |
| ATOM | 4815 | NE | ARG | B | 260 | 40.791 | 29.696 | 27.881 | 1.00 | 64.97 | N |
| ATOM | 4816 | CZ | ARG | B | 260 | 40.005 | 30.118 | 26.899 | 1.00 | 66.33 | C |
| ATOM | 4817 | NH1 | ARG | B | 260 | 40.304 | 31.194 | 26.196 | 1.00 | 76.36 | N |
| ATOM | 4818 | NH2 | ARG | B | 260 | 38.903 | 29.458 | 26.618 | 1.00 | 71.45 | N |
| ATOM | 4819 | C | ARG | B | 260 | 46.109 | 28.569 | 28.457 | 1.00 | 65.95 | C |
| ATOM | 4820 | O | ARG | B | 260 | 46.394 | 28.782 | 29.646 | 1.00 | 61.97 | O |
| ATOM | 4821 | N | LYS | B | 261 | 46.813 | 28.997 | 27.404 | 1.00 | 75.99 | N |
| ATOM | 4822 | CA | LYS | B | 261 | 47.715 | 30.173 | 27.435 | 1.00 | 77.36 | C |
| ATOM | 4823 | CB | LYS | B | 261 | 48.548 | 30.246 | 26.154 | 1.00 | 79.16 | C |
| ATOM | 4824 | CG | LYS | B | 261 | 49.474 | 29.064 | 25.904 | 1.00 | 78.86 | C |
| ATOM | 4825 | CD | LYS | B | 261 | 50.666 | 29.402 | 25.021 | 1.00 | 77.39 | C |
| ATOM | 4826 | CE | LYS | B | 261 | 51.872 | 29.897 | 25.793 | 1.00 | 76.01 | C |
| ATOM | 4827 | NZ | LYS | B | 261 | 52.691 | 28.777 | 26.317 | 1.00 | 82.02 | N |
| ATOM | 4828 | C | LYS | B | 261 | 46.813 | 31.403 | 27.572 | 1.00 | 83.40 | C |
| ATOM | 4829 | O | LYS | B | 261 | 45.900 | 31.528 | 26.738 | 1.00 | 78.55 | O |
| ATOM | 4830 | N | VAL | B | 262 | 47.000 | 32.224 | 28.615 | 1.00 | 83.98 | N |
| ATOM | 4831 | CA | VAL | B | 262 | 46.278 | 33.522 | 28.782 | 1.00 | 78.70 | C |
| ATOM | 4832 | CB | VAL | B | 262 | 45.411 | 33.571 | 30.059 | 1.00 | 77.90 | C |
| ATOM | 4833 | CG1 | VAL | B | 262 | 44.440 | 32.403 | 30.107 | 1.00 | 76.46 | C |
| ATOM | 4834 | CG2 | VAL | B | 262 | 46.229 | 33.645 | 31.339 | 1.00 | 73.98 | C |
| ATOM | 4835 | C | VAL | B | 262 | 47.315 | 34.648 | 28.737 | 1.00 | 78.05 | C |
| ATOM | 4836 | O | VAL | B | 262 | 48.413 | 34.457 | 29.310 | 1.00 | 70.00 | O |
| ATOM | 4837 | N | ARG | B | 263 | 46.977 | 35.740 | 28.036 | 1.00 | 82.03 | N |
| ATOM | 4838 | CA | ARG | B | 263 | 47.800 | 36.971 | 27.876 | 1.00 | 84.81 | C |
| ATOM | 4839 | CB | ARG | B | 263 | 47.825 | 37.432 | 26.415 | 1.00 | 87.11 | C |
| ATOM | 4840 | C | ARG | B | 263 | 47.212 | 38.062 | 28.770 | 1.00 | 85.50 | C |
| ATOM | 4841 | O | ARG | B | 263 | 45.978 | 38.153 | 28.844 | 1.00 | 78.99 | O |
| ATOM | 4842 | N | LEU | B | 264 | 48.067 | 38.869 | 29.398 | 1.00 | 100.08 | N |
| ATOM | 4843 | CA | LEU | B | 264 | 47.663 | 39.871 | 30.419 | 1.00 | 108.73 | C |
| ATOM | 4844 | CB | LEU | B | 264 | 48.682 | 39.842 | 31.561 | 1.00 | 114.03 | C |
| ATOM | 4845 | CG | LEU | B | 264 | 49.099 | 38.447 | 32.027 | 1.00 | 112.57 | C |
| ATOM | 4846 | CD1 | LEU | B | 264 | 50.065 | 38.535 | 33.196 | 1.00 | 110.14 | C |
| ATOM | 4847 | CD2 | LEU | B | 264 | 47.882 | 37.617 | 32.403 | 1.00 | 114.15 | C |
| ATOM | 4848 | C | LEU | B | 264 | 47.569 | 41.260 | 29.779 | 1.00 | 112.40 | C |
| ATOM | 4849 | O | LEU | B | 264 | 46.463 | 41.840 | 29.819 | 1.00 | 107.24 | O |
| ATOM | 4850 | N | GLY | B | 265 | 48.681 | 41.762 | 29.223 | 1.00 | 122.67 | N |
| ATOM | 4851 | CA | GLY | B | 265 | 48.812 | 43.129 | 28.672 | 1.00 | 140.32 | C |
| ATOM | 4852 | C | GLY | B | 265 | 49.789 | 43.983 | 29.471 | 1.00 | 155.36 | C |
| ATOM | 4853 | O | GLY | B | 265 | 50.306 | 43.480 | 30.486 | 1.00 | 166.47 | O |
| ATOM | 4854 | N | HIS | B | 266 | 50.021 | 45.233 | 29.036 | 1.00 | 163.16 | N |
| ATOM | 4855 | CA | HIS | B | 266 | 50.993 | 46.205 | 29.623 | 1.00 | 159.27 | C |
| ATOM | 4856 | CB | HIS | B | 266 | 51.303 | 47.346 | 28.629 | 1.00 | 155.77 | C |
| ATOM | 4857 | CG | HIS | B | 266 | 52.264 | 48.384 | 29.122 | 1.00 | 147.56 | C |
| ATOM | 4858 | ND1 | HIS | B | 266 | 53.637 | 48.194 | 29.110 | 1.00 | 142.57 | N |
| ATOM | 4859 | CE1 | HIS | B | 266 | 54.234 | 49.270 | 29.584 | 1.00 | 138.75 | C |
| ATOM | 4860 | NE2 | HIS | B | 266 | 53.289 | 50.166 | 29.897 | 1.00 | 136.78 | N |
| ATOM | 4861 | CD2 | HIS | B | 266 | 52.062 | 49.630 | 29.609 | 1.00 | 137.27 | C |
| ATOM | 4862 | C | HIS | B | 266 | 50.468 | 46.738 | 30.965 | 1.00 | 157.90 | C |
| ATOM | 4863 | O | HIS | B | 266 | 51.272 | 46.805 | 31.917 | 1.00 | 160.35 | O |
| ATOM | 4864 | N | SER | B | 267 | 49.178 | 47.088 | 31.049 | 1.00 | 147.50 | N |
| ATOM | 4865 | CA | SER | B | 267 | 48.569 | 47.776 | 32.218 | 1.00 | 138.98 | C |
| ATOM | 4866 | CB | SER | B | 267 | 47.123 | 48.108 | 31.959 | 1.00 | 136.06 | C |
| ATOM | 4867 | OG | SER | B | 267 | 46.426 | 46.970 | 31.483 | 1.00 | 131.77 | O |
| ATOM | 4868 | C | SER | B | 267 | 48.739 | 46.941 | 33.497 | 1.00 | 137.64 | C |
| ATOM | 4869 | O | SER | B | 267 | 48.596 | 47.533 | 34.579 | 1.00 | 131.87 | O |
| ATOM | 4870 | N | LEU | B | 268 | 49.048 | 45.638 | 33.383 | 1.00 | 135.45 | N |
| ATOM | 4871 | CA | LEU | B | 268 | 49.212 | 44.699 | 34.532 | 1.00 | 125.18 | C |
| ATOM | 4872 | CB | LEU | B | 268 | 48.175 | 43.576 | 34.433 | 1.00 | 118.69 | C |
| ATOM | 4873 | CG | LEU | B | 268 | 46.722 | 44.025 | 34.303 | 1.00 | 118.09 | C |
| ATOM | 4874 | CD1 | LEU | B | 268 | 46.341 | 44.185 | 32.841 | 1.00 | 125.18 | C |
| ATOM | 4875 | CD2 | LEU | B | 268 | 45.789 | 43.043 | 34.990 | 1.00 | 115.36 | C |
| ATOM | 4876 | C | LEU | B | 268 | 50.619 | 44.086 | 34.585 | 1.00 | 128.46 | C |
| ATOM | 4877 | O | LEU | B | 268 | 50.879 | 43.382 | 35.570 | 1.00 | 123.86 | O |
| ATOM | 4878 | N | LEU | B | 269 | 51.492 | 44.317 | 33.595 | 1.00 | 142.10 | N |
| ATOM | 4879 | CA | LEU | B | 269 | 52.865 | 43.726 | 33.559 | 1.00 | 147.52 | C |
| ATOM | 4880 | CB | LEU | B | 269 | 53.590 | 44.119 | 32.262 | 1.00 | 151.77 | C |
| ATOM | 4881 | CG | LEU | B | 269 | 54.710 | 43.177 | 31.802 | 1.00 | 150.43 | C |
| ATOM | 4882 | CD1 | LEU | B | 269 | 54.741 | 43.054 | 30.284 | 1.00 | 145.09 | C |
| ATOM | 4883 | CD2 | LEU | B | 269 | 56.073 | 43.623 | 32.317 | 1.00 | 147.77 | C |
| ATOM | 4884 | C | LEU | B | 269 | 53.650 | 44.190 | 34.794 | 1.00 | 148.83 | C |
| ATOM | 4885 | O | LEU | B | 269 | 54.651 | 43.529 | 35.131 | 1.00 | 160.88 | O |
| ATOM | 4886 | N | ALA | B | 270 | 53.216 | 45.285 | 35.431 | 1.00 | 141.43 | N |
| ATOM | 4887 | CA | ALA | B | 270 | 53.817 | 45.856 | 36.659 | 1.00 | 140.46 | C |
| ATOM | 4888 | CB | ALA | B | 270 | 53.127 | 47.152 | 37.019 | 1.00 | 138.59 | C |
| ATOM | 4889 | C | ALA | B | 270 | 53.746 | 44.836 | 37.805 | 1.00 | 139.58 | C |
| ATOM | 4890 | O | ALA | B | 270 | 54.779 | 44.642 | 38.484 | 1.00 | 139.32 | O |
| ATOM | 4891 | N | PHE | B | 271 | 52.587 | 44.197 | 38.006 | 1.00 | 135.54 | N |
| ATOM | 4892 | CA | PHE | B | 271 | 52.338 | 43.251 | 39.127 | 1.00 | 125.19 | C |
| ATOM | 4893 | CB | PHE | B | 271 | 50.848 | 43.137 | 39.455 | 1.00 | 123.06 | C |
| ATOM | 4894 | CG | PHE | B | 271 | 50.556 | 42.253 | 40.644 | 1.00 | 132.20 | C |
| ATOM | 4895 | CD1 | PHE | B | 271 | 50.350 | 40.891 | 40.481 | 1.00 | 134.03 | C |
| ATOM | 4896 | CE1 | PHE | B | 271 | 50.093 | 40.074 | 41.572 | 1.00 | 130.39 | C |
| ATOM | 4897 | CZ | PHE | B | 271 | 50.035 | 40.609 | 42.837 | 1.00 | 130.46 | C |
| ATOM | 4898 | CD2 | PHE | B | 271 | 50.506 | 42.775 | 41.930 | 1.00 | 130.88 | C |
| ATOM | 4899 | CE2 | PHE | B | 271 | 50.237 | 41.958 | 43.019 | 1.00 | 133.64 | C |
| ATOM | 4900 | C | PHE | B | 271 | 52.908 | 41.866 | 38.804 | 1.00 | 119.92 | C |
| ATOM | 4901 | O | PHE | B | 271 | 53.558 | 41.288 | 39.687 | 1.00 | 127.25 | O |
| ATOM | 4902 | N | PHE | B | 272 | 52.669 | 41.348 | 37.596 | 1.00 | 117.42 | N |
| ATOM | 4903 | CA | PHE | B | 272 | 52.930 | 39.928 | 37.234 | 1.00 | 121.32 | C |
| ATOM | 4904 | CB | PHE | B | 272 | 51.838 | 39.405 | 36.299 | 1.00 | 120.40 | C |
| ATOM | 4905 | CG | PHE | B | 272 | 50.484 | 39.300 | 36.951 | 1.00 | 112.87 | C |
| ATOM | 4906 | CD1 | PHE | B | 272 | 50.186 | 38.242 | 37.794 | 1.00 | 107.88 | C |
| ATOM | 4907 | CE1 | PHE | B | 272 | 48.943 | 38.151 | 38.401 | 1.00 | 108.24 | C |
| ATOM | 4908 | CZ | PHE | B | 272 | 47.988 | 39.116 | 38.175 | 1.00 | 107.23 | C |
| ATOM | 4909 | CD2 | PHE | B | 272 | 49.515 | 40.269 | 36.739 | 1.00 | 114.81 | C |
| ATOM | 4910 | CE2 | PHE | B | 272 | 48.271 | 40.174 | 37.344 | 1.00 | 110.53 | C |
| ATOM | 4911 | C | PHE | B | 272 | 54.323 | 39.739 | 36.615 | 1.00 | 126.05 | C |
| ATOM | 4912 | O | PHE | B | 272 | 54.831 | 38.603 | 36.711 | 1.00 | 119.84 | O |
| ATOM | 4913 | N | LYS | B | 273 | 54.906 | 40.783 | 36.006 | 1.00 | 131.73 | N |
| ATOM | 4914 | CA | LYS | B | 273 | 56.219 | 40.746 | 35.294 | 1.00 | 134.32 | C |
| ATOM | 4915 | CB | LYS | B | 273 | 57.381 | 40.564 | 36.280 | 1.00 | 133.98 | C |
| ATOM | 4916 | CG | LYS | B | 273 | 57.445 | 41.554 | 37.440 | 1.00 | 130.23 | C |
| ATOM | 4917 | CD | LYS | B | 273 | 58.590 | 41.269 | 38.406 | 1.00 | 123.93 | C |
| ATOM | 4918 | CE | LYS | B | 273 | 58.248 | 41.533 | 39.858 | 1.00 | 117.31 | C |
| ATOM | 4919 | NZ | LYS | B | 273 | 58.127 | 42.983 | 40.144 | 1.00 | 110.48 | N |
| ATOM | 4920 | C | LYS | B | 273 | 56.216 | 39.615 | 34.251 | 1.00 | 134.30 | C |
| ATOM | 4921 | O | LYS | B | 273 | 57.259 | 38.951 | 34.096 | 1.00 | 129.24 | O |
| ATOM | 4922 | N | SER | B | 274 | 55.085 | 39.407 | 33.566 | 1.00 | 140.41 | N |
| ATOM | 4923 | CA | SER | B | 274 | 54.901 | 38.402 | 32.484 | 1.00 | 134.10 | C |
| ATOM | 4924 | CB | SER | B | 274 | 54.643 | 37.033 | 33.048 | 1.00 | 126.23 | C |
| ATOM | 4925 | OG | SER | B | 274 | 55.832 | 36.497 | 33.599 | 1.00 | 119.06 | O |
| ATOM | 4926 | C | SER | B | 274 | 53.766 | 38.835 | 31.552 | 1.00 | 134.88 | C |
| ATOM | 4927 | O | SER | B | 274 | 52.840 | 39.519 | 32.026 | 1.00 | 143.01 | O |
| ATOM | 4928 | N | GLU | B | 275 | 53.846 | 38.450 | 30.275 | 1.00 | 126.22 | N |
| ATOM | 4929 | CA | GLU | B | 275 | 52.816 | 38.751 | 29.247 | 1.00 | 125.19 | C |
| ATOM | 4930 | CB | GLU | B | 275 | 53.485 | 39.174 | 27.934 | 1.00 | 127.15 | C |
| ATOM | 4931 | CG | GLU | B | 275 | 52.525 | 39.817 | 26.939 | 1.00 | 129.01 | C |
| ATOM | 4932 | CD | GLU | B | 275 | 53.156 | 40.795 | 25.958 | 1.00 | 118.73 | C |
| ATOM | 4933 | OE1 | GLU | B | 275 | 53.939 | 41.659 | 26.407 | 1.00 | 106.20 | O |
| ATOM | 4934 | OE2 | GLU | B | 275 | 52.858 | 40.695 | 24.746 | 1.00 | 101.73 | O |
| ATOM | 4935 | C | GLU | B | 275 | 51.898 | 37.531 | 29.081 | 1.00 | 120.92 | C |
| ATOM | 4936 | O | GLU | B | 275 | 50.699 | 37.746 | 28.833 | 1.00 | 116.69 | O |
| ATOM | 4937 | N | LYS | B | 276 | 52.435 | 36.310 | 29.225 | 1.00 | 112.90 | N |
| ATOM | 4938 | CA | LYS | B | 276 | 51.697 | 35.035 | 29.002 | 1.00 | 108.72 | C |
| ATOM | 4939 | CB | LYS | B | 276 | 52.226 | 34.331 | 27.749 | 1.00 | 115.44 | C |
| ATOM | 4940 | CG | LYS | B | 276 | 51.571 | 34.770 | 26.446 | 1.00 | 120.71 | C |
| ATOM | 4941 | CD | LYS | B | 276 | 51.983 | 33.929 | 25.251 | 1.00 | 125.68 | C |
| ATOM | 4942 | CE | LYS | B | 276 | 50.840 | 33.606 | 24.307 | 1.00 | 122.85 | C |
| ATOM | 4943 | NZ | LYS | B | 276 | 50.539 | 34.733 | 23.395 | 1.00 | 120.09 | N |
| ATOM | 4944 | C | LYS | B | 276 | 51.813 | 34.127 | 30.233 | 1.00 | 96.52 | C |
| ATOM | 4945 | O | LYS | B | 276 | 52.905 | 34.057 | 30.825 | 1.00 | 85.69 | O |
| ATOM | 4946 | N | LEU | B | 277 | 50.717 | 33.453 | 30.590 | 1.00 | 89.99 | N |
| ATOM | 4947 | CA | LEU | B | 277 | 50.671 | 32.446 | 31.683 | 1.00 | 90.65 | C |
| ATOM | 4948 | CB | LEU | B | 277 | 49.894 | 33.011 | 32.876 | 1.00 | 93.46 | C |
| ATOM | 4949 | CG | LEU | B | 277 | 50.686 | 33.895 | 33.842 | 1.00 | 94.96 | C |
| ATOM | 4950 | CD1 | LEU | B | 277 | 49.761 | 34.460 | 34.911 | 1.00 | 94.59 | C |
| ATOM | 4951 | CD2 | LEU | B | 277 | 51.846 | 33.144 | 34.491 | 1.00 | 95.19 | C |
| ATOM | 4952 | C | LEU | B | 277 | 50.019 | 31.157 | 31.170 | 1.00 | 84.94 | C |
| ATOM | 4953 | O | LEU | B | 277 | 49.001 | 31.242 | 30.455 | 1.00 | 83.17 | O |
| ATOM | 4954 | N | ASP | B | 278 | 50.605 | 30.014 | 31.533 | 1.00 | 80.72 | N |
| ATOM | 4955 | CA | ASP | B | 278 | 50.058 | 28.652 | 31.303 | 1.00 | 72.61 | C |
| ATOM | 4956 | CB | ASP | B | 278 | 51.197 | 27.641 | 31.187 | 1.00 | 73.81 | C |
| ATOM | 4957 | CG | ASP | B | 278 | 52.063 | 27.887 | 29.968 | 1.00 | 77.44 | C |
| ATOM | 4958 | OD1 | ASP | B | 278 | 51.638 | 28.688 | 29.108 | 1.00 | 80.45 | O |
| ATOM | 4959 | OD2 | ASP | B | 278 | 53.151 | 27.280 | 29.889 | 1.00 | 79.26 | O |
| ATOM | 4960 | C | ASP | B | 278 | 49.107 | 28.325 | 32.455 | 1.00 | 69.99 | C |
| ATOM | 4961 | O | ASP | B | 278 | 49.591 | 28.194 | 33.600 | 1.00 | 72.87 | O |
| ATOM | 4962 | N | MET | B | 279 | 47.809 | 28.219 | 32.167 | 1.00 | 64.86 | N |
| ATOM | 4963 | CA | MET | B | 279 | 46.747 | 28.111 | 33.198 | 1.00 | 64.67 | C |
| ATOM | 4964 | CB | MET | B | 279 | 45.771 | 29.289 | 33.112 | 1.00 | 69.76 | C |
| ATOM | 4965 | CG | MET | B | 279 | 46.443 | 30.659 | 33.206 | 1.00 | 68.81 | C |
| ATOM | 4966 | SD | MET | B | 279 | 47.060 | 31.051 | 34.853 | 1.00 | 62.02 | S |
| ATOM | 4967 | CE | MET | B | 279 | 45.521 | 31.284 | 35.737 | 1.00 | 61.93 | C |
| ATOM | 4968 | C | MET | B | 279 | 45.962 | 26.808 | 33.032 | 1.00 | 66.07 | C |
| ATOM | 4969 | O | MET | B | 279 | 45.805 | 26.313 | 31.889 | 1.00 | 66.01 | O |
| ATOM | 4970 | N | ILE | B | 280 | 45.441 | 26.312 | 34.154 | 1.00 | 62.88 | N |
| ATOM | 4971 | CA | ILE | B | 280 | 44.560 | 25.116 | 34.218 | 1.00 | 54.52 | C |
| ATOM | 4972 | CB | ILE | B | 280 | 45.345 | 23.927 | 34.793 | 1.00 | 55.96 | C |
| ATOM | 4973 | CG1 | ILE | B | 280 | 46.639 | 23.676 | 34.019 | 1.00 | 55.97 | C |
| ATOM | 4974 | CG2 | ILE | B | 280 | 44.469 | 22.688 | 34.854 | 1.00 | 58.37 | C |
| ATOM | 4975 | CD1 | ILE | B | 280 | 46.419 | 23.275 | 32.581 | 1.00 | 57.79 | C |
| ATOM | 4976 | C | ILE | B | 280 | 43.344 | 25.462 | 35.074 | 1.00 | 50.17 | C |
| ATOM | 4977 | O | ILE | B | 280 | 43.517 | 26.048 | 36.137 | 1.00 | 50.14 | O |
| ATOM | 4978 | N | ARG | B | 281 | 42.159 | 25.100 | 34.611 | 1.00 | 47.56 | N |
| ATOM | 4979 | CA | ARG | B | 281 | 40.905 | 25.141 | 35.395 | 1.00 | 48.66 | C |
| ATOM | 4980 | CB | ARG | B | 281 | 39.802 | 25.772 | 34.549 | 1.00 | 48.32 | C |
| ATOM | 4981 | CG | ARG | B | 281 | 38.449 | 25.808 | 35.236 | 1.00 | 53.00 | C |
| ATOM | 4982 | CD | ARG | B | 281 | 37.452 | 26.651 | 34.477 | 1.00 | 52.03 | C |
| ATOM | 4983 | NE | ARG | B | 281 | 36.748 | 25.931 | 33.433 | 1.00 | 55.58 | N |
| ATOM | 4984 | CZ | ARG | B | 281 | 36.127 | 26.485 | 32.392 | 1.00 | 54.90 | C |
| ATOM | 4985 | NH1 | ARG | B | 281 | 36.143 | 27.791 | 32.201 | 1.00 | 56.52 | N |
| ATOM | 4986 | NH2 | ARG | B | 281 | 35.508 | 25.713 | 31.525 | 1.00 | 54.77 | N |
| ATOM | 4987 | C | ARG | B | 281 | 40.545 | 23.702 | 35.782 | 1.00 | 53.04 | C |
| ATOM | 4988 | O | ARG | B | 281 | 40.634 | 22.812 | 34.895 | 1.00 | 55.44 | O |
| ATOM | 4989 | N | LEU | B | 282 | 40.159 | 23.480 | 37.038 | 1.00 | 46.60 | N |
| ATOM | 4990 | CA | LEU | B | 282 | 39.836 | 22.131 | 37.563 | 1.00 | 47.07 | C |
| ATOM | 4991 | CB | LEU | B | 282 | 40.901 | 21.682 | 38.564 | 1.00 | 49.53 | C |
| ATOM | 4992 | CG | LEU | B | 282 | 42.309 | 21.590 | 37.977 | 1.00 | 49.81 | C |
| ATOM | 4993 | CD1 | LEU | B | 282 | 43.178 | 22.721 | 38.476 | 1.00 | 50.99 | C |
| ATOM | 4994 | CD2 | LEU | B | 282 | 42.955 | 20.252 | 38.282 | 1.00 | 52.01 | C |
| ATOM | 4995 | C | LEU | B | 282 | 38.461 | 22.174 | 38.198 | 1.00 | 46.20 | C |
| ATOM | 4996 | O | LEU | B | 282 | 38.066 | 23.265 | 38.622 | 1.00 | 54.79 | O |
| ATOM | 4997 | N | ARG | B | 283 | 37.740 | 21.052 | 38.173 | 1.00 | 45.08 | N |
| ATOM | 4998 | CA | ARG | B | 283 | 36.529 | 20.875 | 39.004 | 1.00 | 47.12 | C |
| ATOM | 4999 | CB | ARG | B | 283 | 35.320 | 20.280 | 38.278 | 1.00 | 48.05 | C |
| ATOM | 5000 | CG | ARG | B | 283 | 34.218 | 19.879 | 39.249 | 1.00 | 48.90 | C |
| ATOM | 5001 | CD | ARG | B | 283 | 32.837 | 19.605 | 38.689 | 1.00 | 59.17 | C |
| ATOM | 5002 | NE | ARG | B | 283 | 31.964 | 20.772 | 38.671 | 1.00 | 64.27 | N |
| ATOM | 5003 | CZ | ARG | B | 283 | 31.846 | 21.608 | 37.640 | 1.00 | 75.18 | C |
| ATOM | 5004 | NH1 | ARG | B | 283 | 31.026 | 22.644 | 37.712 | 1.00 | 68.45 | N |
| ATOM | 5005 | NH2 | ARG | B | 283 | 32.544 | 21.399 | 36.532 | 1.00 | 78.34 | N |
| ATOM | 5006 | C | ARG | B | 283 | 36.900 | 19.980 | 40.183 | 1.00 | 50.38 | C |
| ATOM | 5007 | O | ARG | B | 283 | 37.541 | 18.935 | 39.977 | 1.00 | 55.41 | O |
| ATOM | 5008 | N | ASN | B | 284 | 36.510 | 20.420 | 41.376 | 1.00 | 50.22 | N |
| ATOM | 5009 | CA | ASN | B | 284 | 36.341 | 19.563 | 42.567 | 1.00 | 44.12 | C |
| ATOM | 5010 | CB | ASN | B | 284 | 36.346 | 20.383 | 43.847 | 1.00 | 47.23 | C |
| ATOM | 5011 | CG | ASN | B | 284 | 36.129 | 19.502 | 45.055 | 1.00 | 50.97 | C |
| ATOM | 5012 | OD1 | ASN | B | 284 | 36.160 | 18.281 | 44.948 | 1.00 | 51.42 | O |
| ATOM | 5013 | ND2 | ASN | B | 284 | 35.914 | 20.113 | 46.203 | 1.00 | 52.93 | N |
| ATOM | 5014 | C | ASN | B | 284 | 35.027 | 18.805 | 42.426 | 1.00 | 43.04 | C |
| ATOM | 5015 | O | ASN | B | 284 | 33.963 | 19.420 | 42.478 | 1.00 | 43.15 | O |
| ATOM | 5016 | N | PRO | B | 285 | 35.059 | 17.460 | 42.230 | 1.00 | 43.22 | N |
| ATOM | 5017 | CA | PRO | B | 285 | 33.831 | 16.656 | 42.159 | 1.00 | 41.18 | C |
| ATOM | 5018 | CB | PRO | B | 285 | 34.298 | 15.238 | 41.794 | 1.00 | 39.58 | C |
| ATOM | 5019 | CG | PRO | B | 285 | 35.779 | 15.359 | 41.459 | 1.00 | 42.35 | C |
| ATOM | 5020 | CD | PRO | B | 285 | 36.281 | 16.654 | 42.069 | 1.00 | 40.26 | C |
| ATOM | 5021 | C | PRO | B | 285 | 33.081 | 16.587 | 43.505 | 1.00 | 43.26 | C |
| ATOM | 5022 | O | PRO | B | 285 | 31.868 | 16.417 | 43.508 | 1.00 | 38.81 | O |
| ATOM | 5023 | N | TRP | B | 286 | 33.812 | 16.728 | 44.616 | 1.00 | 42.89 | N |
| ATOM | 5024 | CA | TRP | B | 286 | 33.282 | 16.518 | 45.990 | 1.00 | 53.79 | C |
| ATOM | 5025 | CB | TRP | B | 286 | 34.414 | 16.146 | 46.948 | 1.00 | 56.47 | C |
| ATOM | 5026 | CG | TRP | B | 286 | 34.767 | 14.698 | 46.883 | 1.00 | 57.98 | C |
| ATOM | 5027 | CD1 | TRP | B | 286 | 34.166 | 13.681 | 47.559 | 1.00 | 63.14 | C |
| ATOM | 5028 | NE1 | TRP | B | 286 | 34.759 | 12.490 | 47.246 | 1.00 | 64.56 | N |
| ATOM | 5029 | CE2 | TRP | B | 286 | 35.760 | 12.712 | 46.345 | 1.00 | 63.47 | C |
| ATOM | 5030 | CD2 | TRP | B | 286 | 35.787 | 14.099 | 46.078 | 1.00 | 62.57 | C |
| ATOM | 5031 | CE3 | TRP | B | 286 | 36.726 | 14.591 | 45.167 | 1.00 | 61.30 | C |
| ATOM | 5032 | CZ3 | TRP | B | 286 | 37.596 | 13.702 | 44.570 | 1.00 | 64.92 | C |
| ATOM | 5033 | CH2 | TRP | B | 286 | 37.546 | 12.333 | 44.845 | 1.00 | 59.17 | C |
| ATOM | 5034 | CZ2 | TRP | B | 286 | 36.630 | 11.814 | 45.728 | 1.00 | 62.42 | C |
| ATOM | 5035 | C | TRP | B | 286 | 32.478 | 17.726 | 46.491 | 1.00 | 55.19 | C |
| ATOM | 5036 | O | TRP | B | 286 | 31.721 | 17.535 | 47.447 | 1.00 | 55.65 | O |
| ATOM | 5037 | N | GLY | B | 287 | 32.596 | 18.895 | 45.849 | 1.00 | 59.40 | N |
| ATOM | 5038 | CA | GLY | B | 287 | 31.713 | 20.055 | 46.094 | 1.00 | 60.56 | C |
| ATOM | 5039 | C | GLY | B | 287 | 32.392 | 21.378 | 45.786 | 1.00 | 54.12 | C |
| ATOM | 5040 | O | GLY | B | 287 | 32.983 | 21.490 | 44.724 | 1.00 | 51.93 | O |
| ATOM | 5041 | N | GLU | B | 288 | 32.286 | 22.347 | 46.693 | 1.00 | 55.10 | N |
| ATOM | 5042 | CA | GLU | B | 288 | 32.873 | 23.712 | 46.579 | 1.00 | 50.76 | C |
| ATOM | 5043 | CB | GLU | B | 288 | 32.440 | 24.518 | 47.804 | 1.00 | 52.55 | C |
| ATOM | 5044 | CG | GLU | B | 288 | 33.266 | 25.761 | 48.079 | 1.00 | 60.75 | C |
| ATOM | 5045 | CD | GLU | B | 288 | 32.477 | 26.968 | 48.575 | 1.00 | 68.16 | C |
| ATOM | 5046 | OE1 | GLU | B | 288 | 33.130 | 27.958 | 49.001 | 1.00 | 72.75 | O |
| ATOM | 5047 | OE2 | GLU | B | 288 | 31.213 | 26.944 | 48.490 | 1.00 | 71.52 | O |
| ATOM | 5048 | C | GLU | B | 288 | 34.394 | 23.611 | 46.405 | 1.00 | 44.79 | C |
| ATOM | 5049 | O | GLU | B | 288 | 35.000 | 22.729 | 47.006 | 1.00 | 44.36 | O |
| ATOM | 5050 | N | ARG | B | 289 | 34.983 | 24.444 | 45.550 | 1.00 | 43.73 | N |
| ATOM | 5051 | CA | ARG | B | 289 | 36.453 | 24.504 | 45.382 | 1.00 | 50.15 | C |
| ATOM | 5052 | CB | ARG | B | 289 | 36.872 | 25.612 | 44.408 | 1.00 | 53.38 | C |
| ATOM | 5053 | CG | ARG | B | 289 | 36.428 | 27.025 | 44.777 | 1.00 | 61.83 | C |
| ATOM | 5054 | CD | ARG | B | 289 | 37.477 | 27.829 | 45.542 | 1.00 | 67.30 | C |
| ATOM | 5055 | NE | ARG | B | 289 | 36.930 | 29.070 | 46.087 | 1.00 | 72.37 | N |
| ATOM | 5056 | CZ | ARG | B | 289 | 36.109 | 29.157 | 47.140 | 1.00 | 71.16 | C |
| ATOM | 5057 | NH1 | ARG | B | 289 | 35.666 | 30.342 | 47.516 | 1.00 | 77.00 | N |
| ATOM | 5058 | NH2 | ARG | B | 289 | 35.708 | 28.081 | 47.799 | 1.00 | 61.04 | N |
| ATOM | 5059 | C | ARG | B | 289 | 37.078 | 24.682 | 46.770 | 1.00 | 54.91 | C |
| ATOM | 5060 | O | ARG | B | 289 | 36.397 | 25.248 | 47.653 | 1.00 | 56.62 | O |
| ATOM | 5061 | N | GLU | B | 290 | 38.325 | 24.226 | 46.937 | 1.00 | 60.41 | N |
| ATOM | 5062 | CA | GLU | B | 290 | 39.084 | 24.251 | 48.217 | 1.00 | 60.19 | C |
| ATOM | 5063 | CB | GLU | B | 290 | 39.360 | 22.809 | 48.650 | 1.00 | 66.54 | C |
| ATOM | 5064 | CG | GLU | B | 290 | 38.130 | 21.903 | 48.606 | 1.00 | 71.52 | C |
| ATOM | 5065 | CD | GLU | B | 290 | 38.389 | 20.435 | 48.285 | 1.00 | 69.56 | C |
| ATOM | 5066 | OE1 | GLU | B | 290 | 39.335 | 20.158 | 47.531 | 1.00 | 60.64 | O |
| ATOM | 5067 | OE2 | GLU | B | 290 | 37.630 | 19.571 | 48.792 | 1.00 | 74.49 | O |
| ATOM | 5068 | C | GLU | B | 290 | 40.384 | 25.058 | 48.067 | 1.00 | 58.47 | C |
| ATOM | 5069 | O | GLU | B | 290 | 40.924 | 25.510 | 49.100 | 1.00 | 68.49 | O |
| ATOM | 5070 | N | TRP | B | 291 | 40.871 | 25.225 | 46.835 | 1.00 | 59.86 | N |
| ATOM | 5071 | CA | TRP | B | 291 | 42.138 | 25.918 | 46.459 | 1.00 | 60.19 | C |
| ATOM | 5072 | CB | TRP | B | 291 | 42.223 | 26.034 | 44.935 | 1.00 | 57.87 | C |
| ATOM | 5073 | CG | TRP | B | 291 | 43.402 | 26.783 | 44.395 | 1.00 | 52.45 | C |
| ATOM | 5074 | CD1 | TRP | B | 291 | 43.347 | 27.811 | 43.504 | 1.00 | 48.83 | C |
| ATOM | 5075 | NE1 | TRP | B | 291 | 44.610 | 28.226 | 43.174 | 1.00 | 54.23 | N |
| ATOM | 5076 | CE2 | TRP | B | 291 | 45.525 | 27.465 | 43.848 | 1.00 | 54.06 | C |
| ATOM | 5077 | CD2 | TRP | B | 291 | 44.800 | 26.525 | 44.619 | 1.00 | 52.17 | C |
| ATOM | 5078 | CE3 | TRP | B | 291 | 45.520 | 25.629 | 45.414 | 1.00 | 55.03 | C |
| ATOM | 5079 | CZ3 | TRP | B | 291 | 46.902 | 25.696 | 45.413 | 1.00 | 59.16 | C |
| ATOM | 5080 | CH2 | TRP | B | 291 | 47.591 | 26.642 | 44.648 | 1.00 | 55.20 | C |
| ATOM | 5081 | CZ2 | TRP | B | 291 | 46.919 | 27.536 | 43.849 | 1.00 | 51.68 | C |
| ATOM | 5082 | C | TRP | B | 291 | 42.248 | 27.291 | 47.123 | 1.00 | 58.68 | C |
| ATOM | 5083 | O | TRP | B | 291 | 41.352 | 28.131 | 46.909 | 1.00 | 58.64 | O |
| ATOM | 5084 | N | ASN | B | 292 | 43.352 | 27.480 | 47.854 | 1.00 | 72.32 | N |
| ATOM | 5085 | CA | ASN | B | 292 | 43.707 | 28.646 | 48.717 | 1.00 | 67.58 | C |
| ATOM | 5086 | CB | ASN | B | 292 | 44.478 | 28.207 | 49.966 | 1.00 | 63.83 | C |
| ATOM | 5087 | CG | ASN | B | 292 | 43.567 | 27.894 | 51.126 | 1.00 | 66.90 | C |
| ATOM | 5088 | OD1 | ASN | B | 292 | 42.583 | 28.587 | 51.341 | 1.00 | 81.43 | O |
| ATOM | 5089 | ND2 | ASN | B | 292 | 43.889 | 26.867 | 51.887 | 1.00 | 68.72 | N |
| ATOM | 5090 | C | ASN | B | 292 | 44.644 | 29.618 | 47.996 | 1.00 | 62.05 | C |
| ATOM | 5091 | O | ASN | B | 292 | 44.627 | 30.791 | 48.357 | 1.00 | 64.37 | O |
| ATOM | 5092 | N | GLY | B | 293 | 45.458 | 29.123 | 47.057 | 1.00 | 59.20 | N |
| ATOM | 5093 | CA | GLY | B | 293 | 46.676 | 29.791 | 46.565 | 1.00 | 56.17 | C |
| ATOM | 5094 | C | GLY | B | 293 | 46.423 | 30.763 | 45.414 | 1.00 | 55.21 | C |
| ATOM | 5095 | O | GLY | B | 293 | 45.286 | 31.172 | 45.128 | 1.00 | 48.86 | O |
| ATOM | 5096 | N | PRO | B | 294 | 47.516 | 31.194 | 44.743 | 1.00 | 54.13 | N |
| ATOM | 5097 | CA | PRO | B | 294 | 47.419 | 32.067 | 43.575 | 1.00 | 54.61 | C |
| ATOM | 5098 | CB | PRO | B | 294 | 48.821 | 31.987 | 42.946 | 1.00 | 58.32 | C |
| ATOM | 5099 | CG | PRO | B | 294 | 49.746 | 31.637 | 44.105 | 1.00 | 58.32 | C |
| ATOM | 5100 | CD | PRO | B | 294 | 48.904 | 30.857 | 45.093 | 1.00 | 54.96 | C |
| ATOM | 5101 | C | PRO | B | 294 | 46.344 | 31.610 | 42.571 | 1.00 | 53.36 | C |
| ATOM | 5102 | O | PRO | B | 294 | 46.389 | 30.485 | 42.115 | 1.00 | 58.68 | O |
| ATOM | 5103 | N | TRP | B | 295 | 45.389 | 32.500 | 42.308 | 1.00 | 52.58 | N |
| ATOM | 5104 | CA | TRP | B | 295 | 44.333 | 32.444 | 41.263 | 1.00 | 51.04 | C |
| ATOM | 5105 | CB | TRP | B | 295 | 44.832 | 31.791 | 39.973 | 1.00 | 53.60 | C |
| ATOM | 5106 | CG | TRP | B | 295 | 45.801 | 32.672 | 39.250 | 1.00 | 53.60 | C |
| ATOM | 5107 | CD1 | TRP | B | 295 | 47.164 | 32.647 | 39.332 | 1.00 | 56.72 | C |
| ATOM | 5108 | NE1 | TRP | B | 295 | 47.706 | 33.630 | 38.546 | 1.00 | 59.08 | N |
| ATOM | 5109 | CE2 | TRP | B | 295 | 46.693 | 34.338 | 37.957 | 1.00 | 56.10 | C |
| ATOM | 5110 | CD2 | TRP | B | 295 | 45.474 | 33.774 | 38.389 | 1.00 | 53.95 | C |
| ATOM | 5111 | CE3 | TRP | B | 295 | 44.274 | 34.315 | 37.911 | 1.00 | 62.01 | C |
| ATOM | 5112 | CZ3 | TRP | B | 295 | 44.322 | 35.385 | 37.042 | 1.00 | 60.44 | C |
| ATOM | 5113 | CH2 | TRP | B | 295 | 45.541 | 35.928 | 36.635 | 1.00 | 61.51 | C |
| ATOM | 5114 | CZ2 | TRP | B | 295 | 46.743 | 35.419 | 37.079 | 1.00 | 59.80 | C |
| ATOM | 5115 | C | TRP | B | 295 | 43.051 | 31.821 | 41.809 | 1.00 | 51.20 | C |
| ATOM | 5116 | O | TRP | B | 295 | 42.086 | 31.702 | 41.027 | 1.00 | 54.48 | O |
| ATOM | 5117 | N | SER | B | 296 | 42.986 | 31.553 | 43.115 | 1.00 | 49.39 | N |
| ATOM | 5118 | CA | SER | B | 296 | 41.695 | 31.353 | 43.821 | 1.00 | 49.06 | C |
| ATOM | 5119 | CB | SER | B | 296 | 41.873 | 30.932 | 45.260 | 1.00 | 48.92 | C |
| ATOM | 5120 | OG | SER | B | 296 | 42.911 | 31.675 | 45.873 | 1.00 | 52.05 | O |
| ATOM | 5121 | C | SER | B | 296 | 40.912 | 32.660 | 43.722 | 1.00 | 52.18 | C |
| ATOM | 5122 | O | SER | B | 296 | 41.560 | 33.720 | 43.665 | 1.00 | 55.58 | O |
| ATOM | 5123 | N | ASP | B | 297 | 39.582 | 32.586 | 43.747 | 1.00 | 54.24 | N |
| ATOM | 5124 | CA | ASP | B | 297 | 38.679 | 33.765 | 43.698 | 1.00 | 55.69 | C |
| ATOM | 5125 | CB | ASP | B | 297 | 37.217 | 33.344 | 43.527 | 1.00 | 53.53 | C |
| ATOM | 5126 | CG | ASP | B | 297 | 36.698 | 32.639 | 44.754 | 1.00 | 54.11 | C |
| ATOM | 5127 | OD1 | ASP | B | 297 | 37.515 | 32.403 | 45.652 | 1.00 | 60.45 | O |
| ATOM | 5128 | OD2 | ASP | B | 297 | 35.501 | 32.329 | 44.790 | 1.00 | 61.05 | O |
| ATOM | 5129 | C | ASP | B | 297 | 38.888 | 34.627 | 44.955 | 1.00 | 59.28 | C |
| ATOM | 5130 | O | ASP | B | 297 | 38.405 | 35.799 | 44.956 | 1.00 | 57.25 | O |
| ATOM | 5131 | N | THR | B | 298 | 39.568 | 34.080 | 45.973 | 1.00 | 56.40 | N |
| ATOM | 5132 | CA | THR | B | 298 | 39.936 | 34.792 | 47.230 | 1.00 | 56.57 | C |
| ATOM | 5133 | CB | THR | B | 298 | 39.783 | 33.861 | 48.442 | 1.00 | 57.56 | C |
| ATOM | 5134 | OG1 | THR | B | 298 | 40.829 | 32.892 | 48.375 | 1.00 | 61.38 | O |
| ATOM | 5135 | CG2 | THR | B | 298 | 38.450 | 33.146 | 48.502 | 1.00 | 54.86 | C |
| ATOM | 5136 | C | THR | B | 298 | 41.375 | 35.337 | 47.150 | 1.00 | 54.61 | C |
| ATOM | 5137 | O | THR | B | 298 | 41.773 | 35.992 | 48.111 | 1.00 | 51.99 | O |
| ATOM | 5138 | N | SER | B | 299 | 42.132 | 35.076 | 46.073 | 1.00 | 49.97 | N |
| ATOM | 5139 | CA | SER | B | 299 | 43.569 | 35.459 | 45.951 | 1.00 | 55.39 | C |
| ATOM | 5140 | CB | SER | B | 299 | 44.336 | 34.546 | 45.009 | 1.00 | 57.11 | C |
| ATOM | 5141 | OG | SER | B | 299 | 43.879 | 34.642 | 43.659 | 1.00 | 49.01 | O |
| ATOM | 5142 | C | SER | B | 299 | 43.711 | 36.917 | 45.497 | 1.00 | 59.73 | C |
| ATOM | 5143 | O | SER | B | 299 | 42.734 | 37.500 | 44.987 | 1.00 | 53.64 | O |
| ATOM | 5144 | N | GLU | B | 300 | 44.908 | 37.480 | 45.648 | 1.00 | 68.22 | N |
| ATOM | 5145 | CA | GLU | B | 300 | 45.180 | 38.869 | 45.198 | 1.00 | 73.23 | C |
| ATOM | 5146 | CB | GLU | B | 300 | 46.339 | 39.486 | 45.992 | 1.00 | 75.32 | C |
| ATOM | 5147 | CG | GLU | B | 300 | 47.726 | 39.220 | 45.432 | 1.00 | 82.25 | C |
| ATOM | 5148 | CD | GLU | B | 300 | 48.850 | 39.913 | 46.190 | 1.00 | 87.05 | C |
| ATOM | 5149 | OE1 | GLU | B | 300 | 50.024 | 39.530 | 45.987 | 1.00 | 91.41 | O |
| ATOM | 5150 | OE2 | GLU | B | 300 | 48.553 | 40.829 | 46.989 | 1.00 | 85.34 | O |
| ATOM | 5151 | C | GLU | B | 300 | 45.338 | 38.824 | 43.670 | 1.00 | 70.60 | C |
| ATOM | 5152 | O | GLU | B | 300 | 44.852 | 39.764 | 42.993 | 1.00 | 56.47 | O |
| ATOM | 5153 | N | GLU | B | 301 | 45.931 | 37.744 | 43.143 | 1.00 | 71.41 | N |
| ATOM | 5154 | CA | GLU | B | 301 | 46.113 | 37.546 | 41.675 | 1.00 | 65.43 | C |
| ATOM | 5155 | CB | GLU | B | 301 | 46.706 | 36.174 | 41.346 | 1.00 | 61.61 | C |
| ATOM | 5156 | CG | GLU | B | 301 | 48.196 | 36.058 | 41.640 | 1.00 | 61.86 | C |
| ATOM | 5157 | CD | GLU | B | 301 | 48.548 | 35.742 | 43.085 | 1.00 | 61.93 | C |
| ATOM | 5158 | OE1 | GLU | B | 301 | 47.606 | 35.669 | 43.932 | 1.00 | 60.05 | O |
| ATOM | 5159 | OE2 | GLU | B | 301 | 49.759 | 35.579 | 43.364 | 1.00 | 62.35 | O |
| ATOM | 5160 | C | GLU | B | 301 | 44.749 | 37.723 | 41.003 | 1.00 | 62.98 | C |
| ATOM | 5161 | O | GLU | B | 301 | 44.638 | 38.512 | 40.064 | 1.00 | 64.23 | O |
| ATOM | 5162 | N | TRP | B | 302 | 43.729 | 37.042 | 41.509 | 1.00 | 60.78 | N |
| ATOM | 5163 | CA | TRP | B | 302 | 42.358 | 37.109 | 40.950 | 1.00 | 56.96 | C |
| ATOM | 5164 | CB | TRP | B | 302 | 41.479 | 36.084 | 41.647 | 1.00 | 51.08 | C |
| ATOM | 5165 | CG | TRP | B | 302 | 40.065 | 36.047 | 41.175 | 1.00 | 45.26 | C |
| ATOM | 5166 | CD1 | TRP | B | 302 | 39.029 | 36.801 | 41.634 | 1.00 | 44.38 | C |
| ATOM | 5167 | NE1 | TRP | B | 302 | 37.871 | 36.445 | 41.000 | 1.00 | 44.16 | N |
| ATOM | 5168 | CE2 | TRP | B | 302 | 38.136 | 35.410 | 40.137 | 1.00 | 46.82 | C |
| ATOM | 5169 | CD2 | TRP | B | 302 | 39.516 | 35.142 | 40.207 | 1.00 | 42.56 | C |
| ATOM | 5170 | CE3 | TRP | B | 302 | 40.046 | 34.109 | 39.429 | 1.00 | 47.56 | C |
| ATOM | 5171 | CZ3 | TRP | B | 302 | 39.210 | 33.409 | 38.587 | 1.00 | 48.00 | C |
| ATOM | 5172 | CH2 | TRP | B | 302 | 37.848 | 33.702 | 38.518 | 1.00 | 46.50 | C |
| ATOM | 5173 | CZ2 | TRP | B | 302 | 37.286 | 34.691 | 39.294 | 1.00 | 48.73 | C |
| ATOM | 5174 | C | TRP | B | 302 | 41.798 | 38.519 | 41.115 | 1.00 | 58.67 | C |
| ATOM | 5175 | O | TRP | B | 302 | 41.201 | 39.039 | 40.158 | 1.00 | 61.02 | O |
| ATOM | 5176 | N | GLN | B | 303 | 41.990 | 39.100 | 42.297 | 1.00 | 63.80 | N |
| ATOM | 5177 | CA | GLN | B | 303 | 41.395 | 40.402 | 42.671 | 1.00 | 63.12 | C |
| ATOM | 5178 | CB | GLN | B | 303 | 41.414 | 40.523 | 44.190 | 1.00 | 63.17 | C |
| ATOM | 5179 | CG | GLN | B | 303 | 40.218 | 39.790 | 44.777 | 1.00 | 71.15 | C |
| ATOM | 5180 | CD | GLN | B | 303 | 40.225 | 39.633 | 46.275 | 1.00 | 77.51 | C |
| ATOM | 5181 | OE1 | GLN | B | 303 | 41.063 | 40.193 | 46.981 | 1.00 | 78.17 | O |
| ATOM | 5182 | NE2 | GLN | B | 303 | 39.276 | 38.849 | 46.768 | 1.00 | 76.61 | N |
| ATOM | 5183 | C | GLN | B | 303 | 42.078 | 41.532 | 41.893 | 1.00 | 64.09 | C |
| ATOM | 5184 | O | GLN | B | 303 | 41.409 | 42.552 | 41.670 | 1.00 | 76.21 | O |
| ATOM | 5185 | N | LYS | B | 304 | 43.318 | 41.333 | 41.435 | 1.00 | 65.29 | N |
| ATOM | 5186 | CA | LYS | B | 304 | 44.078 | 42.326 | 40.616 | 1.00 | 74.85 | C |
| ATOM | 5187 | CB | LYS | B | 304 | 45.556 | 41.925 | 40.478 | 1.00 | 81.97 | C |
| ATOM | 5188 | CG | LYS | B | 304 | 46.505 | 43.037 | 40.031 | 1.00 | 94.79 | C |
| ATOM | 5189 | CD | LYS | B | 304 | 46.758 | 44.120 | 41.080 | 1.00 | 106.11 | C |
| ATOM | 5190 | CE | LYS | B | 304 | 47.540 | 45.316 | 40.565 | 1.00 | 110.25 | C |
| ATOM | 5191 | NZ | LYS | B | 304 | 46.695 | 46.253 | 39.783 | 1.00 | 107.42 | N |
| ATOM | 5192 | C | LYS | B | 304 | 43.438 | 42.510 | 39.228 | 1.00 | 67.70 | C |
| ATOM | 5193 | O | LYS | B | 304 | 43.721 | 43.541 | 38.609 | 1.00 | 70.79 | O |
| ATOM | 5194 | N | VAL | B | 305 | 42.597 | 41.576 | 38.771 | 1.00 | 66.20 | N |
| ATOM | 5195 | CA | VAL | B | 305 | 42.007 | 41.545 | 37.396 | 1.00 | 64.52 | C |
| ATOM | 5196 | CB | VAL | B | 305 | 42.372 | 40.229 | 36.675 | 1.00 | 70.74 | C |
| ATOM | 5197 | CG1 | VAL | B | 305 | 41.930 | 40.233 | 35.226 | 1.00 | 71.54 | C |
| ATOM | 5198 | CG2 | VAL | B | 305 | 43.860 | 39.916 | 36.751 | 1.00 | 68.87 | C |
| ATOM | 5199 | C | VAL | B | 305 | 40.486 | 41.723 | 37.496 | 1.00 | 62.20 | C |
| ATOM | 5200 | O | VAL | B | 305 | 39.857 | 41.070 | 38.338 | 1.00 | 61.77 | O |
| ATOM | 5201 | N | SER | B | 306 | 39.909 | 42.569 | 36.646 | 1.00 | 61.64 | N |
| ATOM | 5202 | CA | SER | B | 306 | 38.456 | 42.876 | 36.629 | 1.00 | 61.73 | C |
| ATOM | 5203 | CB | SER | B | 306 | 38.192 | 44.168 | 35.891 | 1.00 | 64.88 | C |
| ATOM | 5204 | OG | SER | B | 306 | 38.789 | 44.139 | 34.597 | 1.00 | 67.42 | O |
| ATOM | 5205 | C | SER | B | 306 | 37.703 | 41.716 | 35.977 | 1.00 | 58.81 | C |
| ATOM | 5206 | O | SER | B | 306 | 38.314 | 41.010 | 35.142 | 1.00 | 60.96 | O |
| ATOM | 5207 | N | LYS | B | 307 | 36.416 | 41.567 | 36.297 | 1.00 | 52.11 | N |
| ATOM | 5208 | CA | LYS | B | 307 | 35.540 | 40.529 | 35.704 | 1.00 | 55.65 | C |
| ATOM | 5209 | CB | LYS | B | 307 | 34.101 | 40.649 | 36.214 | 1.00 | 54.48 | C |
| ATOM | 5210 | CG | LYS | B | 307 | 33.164 | 39.584 | 35.652 | 1.00 | 58.77 | C |
| ATOM | 5211 | CD | LYS | B | 307 | 32.050 | 39.132 | 36.582 | 1.00 | 57.81 | C |
| ATOM | 5212 | CE | LYS | B | 307 | 30.677 | 39.607 | 36.156 | 1.00 | 62.45 | C |
| ATOM | 5213 | NZ | LYS | B | 307 | 29.921 | 38.552 | 35.441 | 1.00 | 66.28 | N |
| ATOM | 5214 | C | LYS | B | 307 | 35.633 | 40.640 | 34.177 | 1.00 | 60.84 | C |
| ATOM | 5215 | O | LYS | B | 307 | 35.882 | 39.605 | 33.517 | 1.00 | 61.76 | O |
| ATOM | 5216 | N | SER | B | 308 | 35.476 | 41.857 | 33.653 | 1.00 | 58.60 | N |
| ATOM | 5217 | CA | SER | B | 308 | 35.636 | 42.225 | 32.221 | 1.00 | 58.63 | C |
| ATOM | 5218 | CB | SER | B | 308 | 35.519 | 43.729 | 32.065 | 1.00 | 62.67 | C |
| ATOM | 5219 | OG | SER | B | 308 | 35.669 | 44.141 | 30.717 | 1.00 | 62.20 | O |
| ATOM | 5220 | C | SER | B | 308 | 36.963 | 41.687 | 31.655 | 1.00 | 54.30 | C |
| ATOM | 5221 | O | SER | B | 308 | 36.931 | 41.048 | 30.586 | 1.00 | 54.71 | O |
| ATOM | 5222 | N | GLU | B | 309 | 38.092 | 41.905 | 32.331 | 1.00 | 52.99 | N |
| ATOM | 5223 | CA | GLU | B | 309 | 39.424 | 41.423 | 31.858 | 1.00 | 58.33 | C |
| ATOM | 5224 | CB | GLU | B | 309 | 40.535 | 41.904 | 32.779 | 1.00 | 62.53 | C |
| ATOM | 5225 | CG | GLU | B | 309 | 41.201 | 43.178 | 32.330 | 1.00 | 71.19 | C |
| ATOM | 5226 | CD | GLU | B | 309 | 42.694 | 42.986 | 32.157 | 1.00 | 79.06 | C |
| ATOM | 5227 | OE1 | GLU | B | 309 | 43.086 | 42.143 | 31.289 | 1.00 | 72.70 | O |
| ATOM | 5228 | OE2 | GLU | B | 309 | 43.453 | 43.636 | 32.921 | 1.00 | 74.55 | O |
| ATOM | 5229 | C | GLU | B | 309 | 39.507 | 39.887 | 31.816 | 1.00 | 65.77 | C |
| ATOM | 5230 | O | GLU | B | 309 | 40.147 | 39.347 | 30.883 | 1.00 | 71.21 | O |
| ATOM | 5231 | N | ARG | B | 310 | 38.961 | 39.203 | 32.826 | 1.00 | 63.46 | N |
| ATOM | 5232 | CA | ARG | B | 310 | 39.020 | 37.721 | 32.927 | 1.00 | 64.06 | C |
| ATOM | 5233 | CB | ARG | B | 310 | 38.514 | 37.204 | 34.280 | 1.00 | 65.54 | C |
| ATOM | 5234 | CG | ARG | B | 310 | 39.280 | 37.728 | 35.485 | 1.00 | 62.02 | C |
| ATOM | 5235 | CD | ARG | B | 310 | 38.946 | 36.991 | 36.758 | 1.00 | 61.37 | C |
| ATOM | 5236 | NE | ARG | B | 310 | 37.515 | 36.879 | 36.990 | 1.00 | 59.62 | N |
| ATOM | 5237 | CZ | ARG | B | 310 | 36.806 | 37.648 | 37.809 | 1.00 | 60.49 | C |
| ATOM | 5238 | NH1 | ARG | B | 310 | 37.386 | 38.626 | 38.489 | 1.00 | 59.14 | N |
| ATOM | 5239 | NH2 | ARG | B | 310 | 35.510 | 37.417 | 37.955 | 1.00 | 62.66 | N |
| ATOM | 5240 | C | ARG | B | 310 | 38.184 | 37.146 | 31.786 | 1.00 | 61.49 | C |
| ATOM | 5241 | O | ARG | B | 310 | 38.711 | 36.314 | 31.059 | 1.00 | 70.42 | O |
| ATOM | 5242 | N | GLU | B | 311 | 36.951 | 37.621 | 31.614 | 1.00 | 58.84 | N |
| ATOM | 5243 | CA | GLU | B | 311 | 36.012 | 37.100 | 30.582 | 1.00 | 64.15 | C |
| ATOM | 5244 | CB | GLU | B | 311 | 34.705 | 37.884 | 30.583 | 1.00 | 63.34 | C |
| ATOM | 5245 | CG | GLU | B | 311 | 33.918 | 37.676 | 31.860 | 1.00 | 70.47 | C |
| ATOM | 5246 | CD | GLU | B | 311 | 32.571 | 38.366 | 31.888 | 1.00 | 72.72 | C |
| ATOM | 5247 | OE1 | GLU | B | 311 | 32.243 | 39.066 | 30.899 | 1.00 | 78.02 | O |
| ATOM | 5248 | OE2 | GLU | B | 311 | 31.852 | 38.184 | 32.884 | 1.00 | 69.44 | O |
| ATOM | 5249 | C | GLU | B | 311 | 36.680 | 37.142 | 29.206 | 1.00 | 66.05 | C |
| ATOM | 5250 | O | GLU | B | 311 | 36.493 | 36.175 | 28.451 | 1.00 | 76.78 | O |
| ATOM | 5251 | N | LYS | B | 312 | 37.449 | 38.194 | 28.908 | 1.00 | 70.83 | N |
| ATOM | 5252 | CA | LYS | B | 312 | 38.168 | 38.336 | 27.614 | 1.00 | 75.16 | C |
| ATOM | 5253 | CB | LYS | B | 312 | 38.687 | 39.765 | 27.401 | 1.00 | 81.06 | C |
| ATOM | 5254 | CG | LYS | B | 312 | 39.506 | 39.991 | 26.126 | 1.00 | 86.87 | C |
| ATOM | 5255 | CD | LYS | B | 312 | 38.772 | 40.705 | 24.994 | 1.00 | 86.57 | C |
| ATOM | 5256 | CE | LYS | B | 312 | 37.468 | 40.043 | 24.596 | 1.00 | 89.60 | C |
| ATOM | 5257 | NZ | LYS | B | 312 | 37.002 | 40.507 | 23.269 | 1.00 | 83.88 | N |
| ATOM | 5258 | C | LYS | B | 312 | 39.300 | 37.307 | 27.591 | 1.00 | 69.97 | C |
| ATOM | 5259 | O | LYS | B | 312 | 39.543 | 36.714 | 26.522 | 1.00 | 78.41 | O |
| ATOM | 5260 | N | MET | B | 313 | 39.949 | 37.084 | 28.731 | 1.00 | 62.52 | N |
| ATOM | 5261 | CA | MET | B | 313 | 41.086 | 36.133 | 28.847 | 1.00 | 59.08 | C |
| ATOM | 5262 | CB | MET | B | 313 | 41.869 | 36.409 | 30.133 | 1.00 | 64.39 | C |
| ATOM | 5263 | CG | MET | B | 313 | 42.697 | 37.663 | 30.123 | 1.00 | 67.71 | C |
| ATOM | 5264 | SD | MET | B | 313 | 43.950 | 37.589 | 31.446 | 1.00 | 84.88 | S |
| ATOM | 5265 | CE | MET | B | 313 | 42.891 | 37.609 | 32.891 | 1.00 | 83.47 | C |
| ATOM | 5266 | C | MET | B | 313 | 40.575 | 34.678 | 28.866 | 1.00 | 52.77 | C |
| ATOM | 5267 | O | MET | B | 313 | 41.418 | 33.756 | 28.879 | 1.00 | 49.26 | O |
| ATOM | 5268 | N | GLY | B | 314 | 39.251 | 34.478 | 28.891 | 1.00 | 51.48 | N |
| ATOM | 5269 | CA | GLY | B | 314 | 38.582 | 33.175 | 29.071 | 1.00 | 54.47 | C |
| ATOM | 5270 | C | GLY | B | 314 | 38.810 | 32.559 | 30.453 | 1.00 | 59.95 | C |
| ATOM | 5271 | O | GLY | B | 314 | 38.746 | 31.313 | 30.551 | 1.00 | 66.65 | O |
| ATOM | 5272 | N | VAL | B | 315 | 39.079 | 33.359 | 31.493 | 1.00 | 53.91 | N |
| ATOM | 5273 | CA | VAL | B | 315 | 39.365 | 32.837 | 32.863 | 1.00 | 49.38 | C |
| ATOM | 5274 | CB | VAL | B | 315 | 40.579 | 33.514 | 33.507 | 1.00 | 49.23 | C |
| ATOM | 5275 | CG1 | VAL | B | 315 | 40.768 | 33.075 | 34.955 | 1.00 | 49.07 | C |
| ATOM | 5276 | CG2 | VAL | B | 315 | 41.832 | 33.245 | 32.695 | 1.00 | 47.59 | C |
| ATOM | 5277 | C | VAL | B | 315 | 38.108 | 32.980 | 33.715 | 1.00 | 46.29 | C |
| ATOM | 5278 | O | VAL | B | 315 | 38.064 | 33.854 | 34.575 | 1.00 | 49.17 | O |
| ATOM | 5279 | N | THR | B | 316 | 37.150 | 32.102 | 33.462 | 1.00 | 46.88 | N |
| ATOM | 5280 | CA | THR | B | 316 | 35.809 | 32.080 | 34.080 | 1.00 | 48.27 | C |
| ATOM | 5281 | CB | THR | B | 316 | 34.765 | 32.403 | 33.012 | 1.00 | 50.65 | C |
| ATOM | 5282 | OG1 | THR | B | 316 | 34.780 | 31.311 | 32.100 | 1.00 | 44.09 | O |
| ATOM | 5283 | CG2 | THR | B | 316 | 35.058 | 33.677 | 32.250 | 1.00 | 58.45 | C |
| ATOM | 5284 | C | THR | B | 316 | 35.551 | 30.697 | 34.701 | 1.00 | 51.79 | C |
| ATOM | 5285 | O | THR | B | 316 | 36.383 | 29.770 | 34.538 | 1.00 | 49.41 | O |
| ATOM | 5286 | N | VAL | B | 317 | 34.415 | 30.558 | 35.373 | 1.00 | 47.40 | N |
| ATOM | 5287 | CA | VAL | B | 317 | 33.977 | 29.285 | 35.998 | 1.00 | 52.17 | C |
| ATOM | 5288 | CB | VAL | B | 317 | 34.330 | 29.266 | 37.496 | 1.00 | 52.38 | C |
| ATOM | 5289 | CG1 | VAL | B | 317 | 35.839 | 29.250 | 37.740 | 1.00 | 50.43 | C |
| ATOM | 5290 | CG2 | VAL | B | 317 | 33.663 | 30.418 | 38.230 | 1.00 | 50.07 | C |
| ATOM | 5291 | C | VAL | B | 317 | 32.472 | 29.187 | 35.784 | 1.00 | 51.88 | C |
| ATOM | 5292 | O | VAL | B | 317 | 31.834 | 30.231 | 35.831 | 1.00 | 53.79 | O |
| ATOM | 5293 | N | GLN | B | 318 | 31.944 | 27.982 | 35.595 | 1.00 | 55.43 | N |
| ATOM | 5294 | CA | GLN | B | 318 | 30.483 | 27.753 | 35.467 | 1.00 | 64.16 | C |
| ATOM | 5295 | CB | GLN | B | 318 | 30.217 | 26.465 | 34.675 | 1.00 | 74.92 | C |
| ATOM | 5296 | CG | GLN | B | 318 | 28.977 | 25.695 | 35.120 | 1.00 | 85.65 | C |
| ATOM | 5297 | CD | GLN | B | 318 | 28.604 | 24.585 | 34.161 | 1.00 | 97.20 | C |
| ATOM | 5298 | OE1 | GLN | B | 318 | 29.227 | 23.519 | 34.122 | 1.00 | 89.00 | O |
| ATOM | 5299 | NE2 | GLN | B | 318 | 27.557 | 24.821 | 33.384 | 1.00 | 100.07 | N |
| ATOM | 5300 | C | GLN | B | 318 | 29.851 | 27.762 | 36.869 | 1.00 | 64.42 | C |
| ATOM | 5301 | O | GLN | B | 318 | 28.642 | 28.064 | 36.943 | 1.00 | 64.92 | O |
| ATOM | 5302 | N | ASP | B | 319 | 30.615 | 27.463 | 37.935 | 1.00 | 57.73 | N |
| ATOM | 5303 | CA | ASP | B | 319 | 30.072 | 27.377 | 39.316 | 1.00 | 54.48 | C |
| ATOM | 5304 | CB | ASP | B | 319 | 29.208 | 26.128 | 39.482 | 1.00 | 62.11 | C |
| ATOM | 5305 | CG | ASP | B | 319 | 29.967 | 24.876 | 39.082 | 1.00 | 67.74 | C |
| ATOM | 5306 | OD1 | ASP | B | 319 | 31.091 | 24.682 | 39.601 | 1.00 | 74.78 | O |
| ATOM | 5307 | OD2 | ASP | B | 319 | 29.457 | 24.125 | 38.212 | 1.00 | 76.16 | O |
| ATOM | 5308 | C | ASP | B | 319 | 31.188 | 27.279 | 40.355 | 1.00 | 58.91 | C |
| ATOM | 5309 | O | ASP | B | 319 | 32.363 | 27.254 | 39.971 | 1.00 | 54.67 | O |
| ATOM | 5310 | N | ASP | B | 320 | 30.752 | 27.195 | 41.617 | 1.00 | 60.59 | N |
| ATOM | 5311 | CA | ASP | B | 320 | 31.488 | 26.995 | 42.892 | 1.00 | 60.12 | C |
| ATOM | 5312 | CB | ASP | B | 320 | 30.483 | 26.656 | 44.003 | 1.00 | 71.78 | C |
| ATOM | 5313 | CG | ASP | B | 320 | 30.292 | 27.755 | 45.038 | 1.00 | 82.19 | C |
| ATOM | 5314 | OD1 | ASP | B | 320 | 31.312 | 28.373 | 45.430 | 1.00 | 78.62 | O |
| ATOM | 5315 | OD2 | ASP | B | 320 | 29.131 | 27.972 | 45.464 | 1.00 | 85.58 | O |
| ATOM | 5316 | C | ASP | B | 320 | 32.524 | 25.858 | 42.841 | 1.00 | 57.51 | C |
| ATOM | 5317 | O | ASP | B | 320 | 33.545 | 25.974 | 43.569 | 1.00 | 50.37 | O |
| ATOM | 5318 | N | GLY | B | 321 | 32.254 | 24.766 | 42.110 | 1.00 | 49.58 | N |
| ATOM | 5319 | CA | GLY | B | 321 | 33.166 | 23.608 | 42.005 | 1.00 | 44.64 | C |
| ATOM | 5320 | C | GLY | B | 321 | 34.472 | 23.935 | 41.291 | 1.00 | 45.39 | C |
| ATOM | 5321 | O | GLY | B | 321 | 35.490 | 23.265 | 41.570 | 1.00 | 41.96 | O |
| ATOM | 5322 | N | GLU | B | 322 | 34.476 | 24.894 | 40.361 | 1.00 | 45.49 | N |
| ATOM | 5323 | CA | GLU | B | 322 | 35.642 | 25.095 | 39.458 | 1.00 | 49.33 | C |
| ATOM | 5324 | CB | GLU | B | 322 | 35.194 | 25.582 | 38.085 | 1.00 | 48.59 | C |
| ATOM | 5325 | CG | GLU | B | 322 | 34.316 | 24.575 | 37.378 | 1.00 | 51.66 | C |
| ATOM | 5326 | CD | GLU | B | 322 | 34.367 | 24.686 | 35.866 | 1.00 | 55.45 | C |
| ATOM | 5327 | OE1 | GLU | B | 322 | 33.961 | 25.757 | 35.323 | 1.00 | 52.17 | O |
| ATOM | 5328 | OE2 | GLU | B | 322 | 34.822 | 23.708 | 35.237 | 1.00 | 53.24 | O |
| ATOM | 5329 | C | GLU | B | 322 | 36.644 | 26.061 | 40.085 | 1.00 | 46.90 | C |
| ATOM | 5330 | O | GLU | B | 322 | 36.254 | 26.887 | 40.907 | 1.00 | 50.32 | O |
| ATOM | 5331 | N | PHE | B | 323 | 37.900 | 25.983 | 39.670 | 1.00 | 44.41 | N |
| ATOM | 5332 | CA | PHE | B | 323 | 38.948 | 26.883 | 40.183 | 1.00 | 43.24 | C |
| ATOM | 5333 | CB | PHE | B | 323 | 39.288 | 26.506 | 41.623 | 1.00 | 43.41 | C |
| ATOM | 5334 | CG | PHE | B | 323 | 39.852 | 25.120 | 41.808 | 1.00 | 42.81 | C |
| ATOM | 5335 | CD1 | PHE | B | 323 | 39.005 | 24.024 | 41.879 | 1.00 | 40.41 | C |
| ATOM | 5336 | CE1 | PHE | B | 323 | 39.519 | 22.750 | 42.048 | 1.00 | 43.20 | C |
| ATOM | 5337 | CZ | PHE | B | 323 | 40.881 | 22.560 | 42.170 | 1.00 | 43.00 | C |
| ATOM | 5338 | CD2 | PHE | B | 323 | 41.219 | 24.918 | 41.949 | 1.00 | 40.94 | C |
| ATOM | 5339 | CE2 | PHE | B | 323 | 41.733 | 23.641 | 42.115 | 1.00 | 42.40 | C |
| ATOM | 5340 | C | PHE | B | 323 | 40.167 | 26.827 | 39.271 | 1.00 | 44.87 | C |
| ATOM | 5341 | O | PHE | B | 323 | 40.427 | 25.748 | 38.724 | 1.00 | 47.12 | O |
| ATOM | 5342 | N | TRP | B | 324 | 40.893 | 27.950 | 39.184 | 1.00 | 43.77 | N |
| ATOM | 5343 | CA | TRP | B | 324 | 42.062 | 28.157 | 38.293 | 1.00 | 42.75 | C |
| ATOM | 5344 | CB | TRP | B | 324 | 41.946 | 29.520 | 37.608 | 1.00 | 40.88 | C |
| ATOM | 5345 | CG | TRP | B | 324 | 40.897 | 29.547 | 36.553 | 1.00 | 37.95 | C |
| ATOM | 5346 | CD1 | TRP | B | 324 | 39.574 | 29.839 | 36.693 | 1.00 | 41.13 | C |
| ATOM | 5347 | NE1 | TRP | B | 324 | 38.933 | 29.731 | 35.489 | 1.00 | 42.05 | N |
| ATOM | 5348 | CE2 | TRP | B | 324 | 39.851 | 29.370 | 34.532 | 1.00 | 41.14 | C |
| ATOM | 5349 | CD2 | TRP | B | 324 | 41.097 | 29.224 | 35.175 | 1.00 | 38.69 | C |
| ATOM | 5350 | CE3 | TRP | B | 324 | 42.215 | 28.857 | 34.426 | 1.00 | 39.64 | C |
| ATOM | 5351 | CZ3 | TRP | B | 324 | 42.055 | 28.633 | 33.081 | 1.00 | 38.09 | C |
| ATOM | 5352 | CH2 | TRP | B | 324 | 40.811 | 28.765 | 32.471 | 1.00 | 35.04 | C |
| ATOM | 5353 | CZ2 | TRP | B | 324 | 39.693 | 29.144 | 33.168 | 1.00 | 36.85 | C |
| ATOM | 5354 | C | TRP | B | 324 | 43.369 | 28.049 | 39.076 | 1.00 | 45.66 | C |
| ATOM | 5355 | O | TRP | B | 324 | 43.408 | 28.529 | 40.222 | 1.00 | 48.14 | O |
| ATOM | 5356 | N | MET | B | 325 | 44.409 | 27.501 | 38.442 | 1.00 | 47.15 | N |
| ATOM | 5357 | CA | MET | B | 325 | 45.790 | 27.418 | 38.983 | 1.00 | 52.18 | C |
| ATOM | 5358 | CB | MET | B | 325 | 46.099 | 26.068 | 39.643 | 1.00 | 52.19 | C |
| ATOM | 5359 | CG | MET | B | 325 | 45.158 | 25.654 | 40.767 | 1.00 | 52.82 | C |
| ATOM | 5360 | SD | MET | B | 325 | 45.452 | 23.935 | 41.312 | 1.00 | 52.87 | S |
| ATOM | 5361 | CE | MET | B | 325 | 47.194 | 24.017 | 41.747 | 1.00 | 49.91 | C |
| ATOM | 5362 | C | MET | B | 325 | 46.758 | 27.586 | 37.815 | 1.00 | 58.90 | C |
| ATOM | 5363 | O | MET | B | 325 | 46.414 | 27.164 | 36.691 | 1.00 | 61.54 | O |
| ATOM | 5364 | N | THR | B | 326 | 47.928 | 28.162 | 38.079 | 1.00 | 59.92 | N |
| ATOM | 5365 | CA | THR | B | 326 | 49.035 | 28.220 | 37.100 | 1.00 | 66.89 | C |
| ATOM | 5366 | CB | THR | B | 326 | 50.098 | 29.253 | 37.502 | 1.00 | 66.79 | C |
| ATOM | 5367 | OG1 | THR | B | 326 | 50.620 | 28.875 | 38.775 | 1.00 | 66.24 | O |
| ATOM | 5368 | CG2 | THR | B | 326 | 49.563 | 30.670 | 37.554 | 1.00 | 65.38 | C |
| ATOM | 5369 | C | THR | B | 326 | 49.585 | 26.796 | 36.971 | 1.00 | 69.89 | C |
| ATOM | 5370 | O | THR | B | 326 | 49.590 | 26.070 | 37.988 | 1.00 | 67.44 | O |
| ATOM | 5371 | N | PHE | B | 327 | 50.034 | 26.414 | 35.776 | 1.00 | 67.04 | N |
| ATOM | 5372 | CA | PHE | B | 327 | 50.662 | 25.091 | 35.538 | 1.00 | 68.68 | C |
| ATOM | 5373 | CB | PHE | B | 327 | 51.067 | 24.877 | 34.074 | 1.00 | 63.22 | C |
| ATOM | 5374 | CG | PHE | B | 327 | 51.283 | 23.419 | 33.755 | 1.00 | 62.01 | C |
| ATOM | 5375 | CD1 | PHE | B | 327 | 50.262 | 22.496 | 33.951 | 1.00 | 62.62 | C |
| ATOM | 5376 | CE1 | PHE | B | 327 | 50.465 | 21.144 | 33.713 | 1.00 | 61.13 | C |
| ATOM | 5377 | CZ | PHE | B | 327 | 51.691 | 20.700 | 33.272 | 1.00 | 61.69 | C |
| ATOM | 5378 | CD2 | PHE | B | 327 | 52.518 | 22.951 | 33.338 | 1.00 | 57.97 | C |
| ATOM | 5379 | CE2 | PHE | B | 327 | 52.716 | 21.601 | 33.087 | 1.00 | 58.85 | C |
| ATOM | 5380 | C | PHE | B | 327 | 51.847 | 24.926 | 36.496 | 1.00 | 71.80 | C |
| ATOM | 5381 | O | PHE | B | 327 | 52.183 | 23.771 | 36.810 | 1.00 | 74.14 | O |
| ATOM | 5382 | N | GLU | B | 328 | 52.445 | 26.031 | 36.955 | 1.00 | 72.84 | N |
| ATOM | 5383 | CA | GLU | B | 328 | 53.536 | 26.013 | 37.963 | 1.00 | 75.04 | C |
| ATOM | 5384 | CB | GLU | B | 328 | 54.114 | 27.419 | 38.144 | 1.00 | 83.01 | C |
| ATOM | 5385 | CG | GLU | B | 328 | 55.426 | 27.449 | 38.918 | 1.00 | 91.60 | C |
| ATOM | 5386 | CD | GLU | B | 328 | 56.222 | 28.752 | 38.891 | 1.00 | 90.37 | C |
| ATOM | 5387 | OE1 | GLU | B | 328 | 55.807 | 29.720 | 38.199 | 1.00 | 79.58 | O |
| ATOM | 5388 | OE2 | GLU | B | 328 | 57.277 | 28.790 | 39.556 | 1.00 | 90.93 | O |
| ATOM | 5389 | C | GLU | B | 328 | 52.980 | 25.430 | 39.270 | 1.00 | 74.30 | C |
| ATOM | 5390 | O | GLU | B | 328 | 53.584 | 24.489 | 39.799 | 1.00 | 70.51 | O |
| ATOM | 5391 | N | ASP | B | 329 | 51.862 | 25.959 | 39.767 | 1.00 | 72.02 | N |
| ATOM | 5392 | CA | ASP | B | 329 | 51.272 | 25.549 | 41.069 | 1.00 | 71.89 | C |
| ATOM | 5393 | CB | ASP | B | 329 | 50.192 | 26.526 | 41.547 | 1.00 | 77.09 | C |
| ATOM | 5394 | CG | ASP | B | 329 | 50.733 | 27.849 | 42.073 | 1.00 | 82.26 | C |
| ATOM | 5395 | OD1 | ASP | B | 329 | 51.795 | 27.827 | 42.743 | 1.00 | 82.87 | O |
| ATOM | 5396 | OD2 | ASP | B | 329 | 50.084 | 28.895 | 41.813 | 1.00 | 83.18 | O |
| ATOM | 5397 | C | ASP | B | 329 | 50.719 | 24.127 | 40.939 | 1.00 | 71.25 | C |
| ATOM | 5398 | O | ASP | B | 329 | 50.778 | 23.388 | 41.929 | 1.00 | 68.14 | O |
| ATOM | 5399 | N | VAL | B | 330 | 50.201 | 23.760 | 39.764 | 1.00 | 72.53 | N |
| ATOM | 5400 | CA | VAL | B | 330 | 49.688 | 22.385 | 39.476 | 1.00 | 74.26 | C |
| ATOM | 5401 | CB | VAL | B | 330 | 49.050 | 22.296 | 38.074 | 1.00 | 68.60 | C |
| ATOM | 5402 | CG1 | VAL | B | 330 | 48.935 | 20.859 | 37.580 | 1.00 | 74.40 | C |
| ATOM | 5403 | CG2 | VAL | B | 330 | 47.689 | 22.970 | 38.042 | 1.00 | 63.15 | C |
| ATOM | 5404 | C | VAL | B | 330 | 50.839 | 21.377 | 39.670 | 1.00 | 75.66 | C |
| ATOM | 5405 | O | VAL | B | 330 | 50.651 | 20.406 | 40.433 | 1.00 | 76.28 | O |
| ATOM | 5406 | N | CYS | B | 331 | 51.997 | 21.622 | 39.048 | 1.00 | 71.34 | N |
| ATOM | 5407 | CA | CYS | B | 331 | 53.229 | 20.798 | 39.184 | 1.00 | 73.66 | C |
| ATOM | 5408 | CB | CYS | B | 331 | 54.283 | 21.231 | 38.169 | 1.00 | 74.47 | C |
| ATOM | 5409 | SG | CYS | B | 331 | 53.877 | 20.759 | 36.463 | 1.00 | 80.07 | S |
| ATOM | 5410 | C | CYS | B | 331 | 53.782 | 20.858 | 40.620 | 1.00 | 75.60 | C |
| ATOM | 5411 | O | CYS | B | 331 | 54.457 | 19.888 | 41.024 | 1.00 | 77.49 | O |
| ATOM | 5412 | N | ARG | B | 332 | 53.502 | 21.930 | 41.374 | 1.00 | 78.97 | N |
| ATOM | 5413 | CA | ARG | B | 332 | 54.085 | 22.176 | 42.726 | 1.00 | 82.64 | C |
| ATOM | 5414 | CB | ARG | B | 332 | 54.104 | 23.681 | 43.039 | 1.00 | 91.21 | C |
| ATOM | 5415 | CG | ARG | B | 332 | 54.752 | 24.062 | 44.368 | 1.00 | 97.70 | C |
| ATOM | 5416 | CD | ARG | B | 332 | 54.967 | 25.566 | 44.531 | 1.00 | 105.10 | C |
| ATOM | 5417 | NE | ARG | B | 332 | 56.018 | 26.108 | 43.659 | 1.00 | 115.02 | N |
| ATOM | 5418 | CZ | ARG | B | 332 | 55.867 | 27.027 | 42.686 | 1.00 | 112.62 | C |
| ATOM | 5419 | NH1 | ARG | B | 332 | 54.686 | 27.560 | 42.415 | 1.00 | 115.17 | N |
| ATOM | 5420 | NH2 | ARG | B | 332 | 56.919 | 27.413 | 41.980 | 1.00 | 103.35 | N |
| ATOM | 5421 | C | ARG | B | 332 | 53.327 | 21.367 | 43.793 | 1.00 | 78.04 | C |
| ATOM | 5422 | O | ARG | B | 332 | 53.984 | 20.911 | 44.745 | 1.00 | 75.66 | O |
| ATOM | 5423 | N | TYR | B | 333 | 52.011 | 21.173 | 43.650 | 1.00 | 73.70 | N |
| ATOM | 5424 | CA | TYR | B | 333 | 51.138 | 20.621 | 44.722 | 1.00 | 71.09 | C |
| ATOM | 5425 | CB | TYR | B | 333 | 50.043 | 21.626 | 45.083 | 1.00 | 77.14 | C |
| ATOM | 5426 | CG | TYR | B | 333 | 50.530 | 22.873 | 45.776 | 1.00 | 81.36 | C |
| ATOM | 5427 | CD1 | TYR | B | 333 | 50.962 | 23.969 | 45.051 | 1.00 | 82.85 | C |
| ATOM | 5428 | CE1 | TYR | B | 333 | 51.412 | 25.121 | 45.676 | 1.00 | 92.55 | C |
| ATOM | 5429 | CZ | TYR | B | 333 | 51.421 | 25.198 | 47.057 | 1.00 | 96.47 | C |
| ATOM | 5430 | OH | TYR | B | 333 | 51.861 | 26.338 | 47.671 | 1.00 | 95.52 | O |
| ATOM | 5431 | CE2 | TYR | B | 333 | 50.988 | 24.113 | 47.802 | 1.00 | 93.65 | C |
| ATOM | 5432 | CD2 | TYR | B | 333 | 50.547 | 22.966 | 47.159 | 1.00 | 85.82 | C |
| ATOM | 5433 | C | TYR | B | 333 | 50.511 | 19.277 | 44.326 | 1.00 | 68.82 | C |
| ATOM | 5434 | O | TYR | B | 333 | 50.049 | 18.575 | 45.238 | 1.00 | 64.56 | O |
| ATOM | 5435 | N | PHE | B | 334 | 50.446 | 18.936 | 43.034 | 1.00 | 64.62 | N |
| ATOM | 5436 | CA | PHE | B | 334 | 49.887 | 17.641 | 42.563 | 1.00 | 62.66 | C |
| ATOM | 5437 | CB | PHE | B | 334 | 49.048 | 17.829 | 41.303 | 1.00 | 58.57 | C |
| ATOM | 5438 | CG | PHE | B | 334 | 47.668 | 18.405 | 41.506 | 1.00 | 60.45 | C |
| ATOM | 5439 | CD1 | PHE | B | 334 | 46.703 | 17.693 | 42.194 | 1.00 | 57.98 | C |
| ATOM | 5440 | CE1 | PHE | B | 334 | 45.424 | 18.197 | 42.345 | 1.00 | 57.85 | C |
| ATOM | 5441 | CZ | PHE | B | 334 | 45.086 | 19.408 | 41.793 | 1.00 | 54.78 | C |
| ATOM | 5442 | CD2 | PHE | B | 334 | 47.305 | 19.622 | 40.943 | 1.00 | 57.92 | C |
| ATOM | 5443 | CE2 | PHE | B | 334 | 46.022 | 20.117 | 41.085 | 1.00 | 52.18 | C |
| ATOM | 5444 | C | PHE | B | 334 | 51.038 | 16.659 | 42.322 | 1.00 | 63.67 | C |
| ATOM | 5445 | O | PHE | B | 334 | 52.163 | 17.106 | 42.047 | 1.00 | 67.40 | O |
| ATOM | 5446 | N | THR | B | 335 | 50.758 | 15.358 | 42.412 | 1.00 | 61.93 | N |
| ATOM | 5447 | CA | THR | B | 335 | 51.770 | 14.272 | 42.320 | 1.00 | 64.90 | C |
| ATOM | 5448 | CB | THR | B | 335 | 51.680 | 13.330 | 43.526 | 1.00 | 71.18 | C |
| ATOM | 5449 | OG1 | THR | B | 335 | 50.385 | 12.729 | 43.493 | 1.00 | 73.67 | O |
| ATOM | 5450 | CG2 | THR | B | 335 | 51.918 | 14.037 | 44.846 | 1.00 | 68.05 | C |
| ATOM | 5451 | C | THR | B | 335 | 51.627 | 13.510 | 40.998 | 1.00 | 65.18 | C |
| ATOM | 5452 | O | THR | B | 335 | 52.659 | 13.055 | 40.487 | 1.00 | 70.78 | O |
| ATOM | 5453 | N | ASP | B | 336 | 50.416 | 13.375 | 40.454 | 1.00 | 67.77 | N |
| ATOM | 5454 | CA | ASP | B | 336 | 50.176 | 12.554 | 39.239 | 1.00 | 71.33 | C |
| ATOM | 5455 | CB | ASP | B | 336 | 49.658 | 11.166 | 39.613 | 1.00 | 72.80 | C |
| ATOM | 5456 | CG | ASP | B | 336 | 50.603 | 10.408 | 40.528 | 1.00 | 77.25 | C |
| ATOM | 5457 | OD1 | ASP | B | 336 | 51.687 | 9.991 | 40.039 | 1.00 | 75.12 | O |
| ATOM | 5458 | OD2 | ASP | B | 336 | 50.263 | 10.272 | 41.727 | 1.00 | 70.36 | O |
| ATOM | 5459 | C | ASP | B | 336 | 49.201 | 13.277 | 38.309 | 1.00 | 70.61 | C |
| ATOM | 5460 | O | ASP | B | 336 | 48.323 | 14.003 | 38.819 | 1.00 | 68.89 | O |
| ATOM | 5461 | N | ILE | B | 337 | 49.370 | 13.069 | 37.000 | 1.00 | 64.87 | N |
| ATOM | 5462 | CA | ILE | B | 337 | 48.449 | 13.537 | 35.926 | 1.00 | 59.78 | C |
| ATOM | 5463 | CB | ILE | B | 337 | 49.170 | 14.551 | 35.024 | 1.00 | 63.01 | C |
| ATOM | 5464 | CG1 | ILE | B | 337 | 49.621 | 15.782 | 35.807 | 1.00 | 65.16 | C |
| ATOM | 5465 | CG2 | ILE | B | 337 | 48.313 | 14.941 | 33.832 | 1.00 | 66.31 | C |
| ATOM | 5466 | CD1 | ILE | B | 337 | 48.530 | 16.791 | 36.028 | 1.00 | 65.97 | C |
| ATOM | 5467 | C | ILE | B | 337 | 47.954 | 12.318 | 35.140 | 1.00 | 59.25 | C |
| ATOM | 5468 | O | ILE | B | 337 | 48.783 | 11.482 | 34.746 | 1.00 | 62.75 | O |
| ATOM | 5469 | N | ILE | B | 338 | 46.646 | 12.219 | 34.928 | 1.00 | 61.01 | N |
| ATOM | 5470 | CA | ILE | B | 338 | 46.007 | 11.160 | 34.095 | 1.00 | 66.28 | C |
| ATOM | 5471 | CB | ILE | B | 338 | 44.839 | 10.478 | 34.847 | 1.00 | 69.03 | C |
| ATOM | 5472 | CG1 | ILE | B | 338 | 45.314 | 9.740 | 36.104 | 1.00 | 73.14 | C |
| ATOM | 5473 | CG2 | ILE | B | 338 | 44.064 | 9.553 | 33.918 | 1.00 | 67.12 | C |
| ATOM | 5474 | CD1 | ILE | B | 338 | 46.301 | 8.615 | 35.836 | 1.00 | 73.95 | C |
| ATOM | 5475 | C | ILE | B | 338 | 45.569 | 11.808 | 32.774 | 1.00 | 62.60 | C |
| ATOM | 5476 | O | ILE | B | 338 | 44.665 | 12.649 | 32.795 | 1.00 | 62.94 | O |
| ATOM | 5477 | N | LYS | B | 339 | 46.222 | 11.435 | 31.676 | 1.00 | 67.29 | N |
| ATOM | 5478 | CA | LYS | B | 339 | 45.918 | 11.901 | 30.298 | 1.00 | 70.10 | C |
| ATOM | 5479 | CB | LYS | B | 339 | 47.204 | 12.332 | 29.590 | 1.00 | 77.24 | C |
| ATOM | 5480 | CG | LYS | B | 339 | 47.012 | 12.864 | 28.180 | 1.00 | 90.74 | C |
| ATOM | 5481 | CD | LYS | B | 339 | 48.313 | 13.136 | 27.431 | 1.00 | 99.30 | C |
| ATOM | 5482 | CE | LYS | B | 339 | 49.114 | 11.893 | 27.089 | 1.00 | 101.53 | C |
| ATOM | 5483 | NZ | LYS | B | 339 | 48.310 | 10.872 | 26.369 | 1.00 | 105.29 | N |
| ATOM | 5484 | C | LYS | B | 339 | 45.240 | 10.755 | 29.553 | 1.00 | 64.94 | C |
| ATOM | 5485 | O | LYS | B | 339 | 45.842 | 9.672 | 29.453 | 1.00 | 77.83 | O |
| ATOM | 5486 | N | CYS | B | 340 | 44.018 | 10.971 | 29.091 | 1.00 | 57.36 | N |
| ATOM | 5487 | CA | CYS | B | 340 | 43.244 | 9.956 | 28.352 | 1.00 | 54.41 | C |
| ATOM | 5488 | CB | CYS | B | 340 | 42.009 | 9.523 | 29.121 | 1.00 | 48.97 | C |
| ATOM | 5489 | SG | CYS | B | 340 | 41.038 | 8.285 | 28.226 | 1.00 | 48.65 | S |
| ATOM | 5490 | C | CYS | B | 340 | 42.866 | 10.558 | 27.004 | 1.00 | 60.46 | C |
| ATOM | 5491 | O | CYS | B | 340 | 41.868 | 11.293 | 26.965 | 1.00 | 57.01 | O |
| ATOM | 5492 | N | ARG | B | 341 | 43.690 | 10.309 | 25.977 | 1.00 | 66.92 | N |
| ATOM | 5493 | CA | ARG | B | 341 | 43.429 | 10.745 | 24.581 | 1.00 | 64.24 | C |
| ATOM | 5494 | CB | ARG | B | 341 | 44.464 | 10.241 | 23.571 | 1.00 | 69.11 | C |
| ATOM | 5495 | CG | ARG | B | 341 | 45.912 | 10.588 | 23.875 | 1.00 | 77.04 | C |
| ATOM | 5496 | CD | ARG | B | 341 | 46.737 | 10.553 | 22.599 | 1.00 | 84.49 | C |
| ATOM | 5497 | NE | ARG | B | 341 | 46.402 | 9.403 | 21.764 | 1.00 | 89.00 | N |
| ATOM | 5498 | CZ | ARG | B | 341 | 46.976 | 8.204 | 21.839 | 1.00 | 83.44 | C |
| ATOM | 5499 | NH1 | ARG | B | 341 | 47.945 | 7.972 | 22.712 | 1.00 | 84.17 | N |
| ATOM | 5500 | NH2 | ARG | B | 341 | 46.569 | 7.234 | 21.038 | 1.00 | 77.19 | N |
| ATOM | 5501 | C | ARG | B | 341 | 42.120 | 10.099 | 24.173 | 1.00 | 57.89 | C |
| ATOM | 5502 | O | ARG | B | 341 | 41.965 | 8.921 | 24.495 | 1.00 | 61.41 | O |
| ATOM | 5503 | N | VAL | B | 342 | 41.235 | 10.803 | 23.478 | 1.00 | 53.95 | N |
| ATOM | 5504 | CA | VAL | B | 342 | 40.047 | 10.123 | 22.897 | 1.00 | 56.04 | C |
| ATOM | 5505 | CB | VAL | B | 342 | 38.757 | 10.942 | 23.026 | 1.00 | 54.76 | C |
| ATOM | 5506 | CG1 | VAL | B | 342 | 37.660 | 10.422 | 22.116 | 1.00 | 60.33 | C |
| ATOM | 5507 | CG2 | VAL | B | 342 | 38.280 | 10.922 | 24.465 | 1.00 | 61.60 | C |
| ATOM | 5508 | C | VAL | B | 342 | 40.383 | 9.736 | 21.459 | 1.00 | 58.63 | C |
| ATOM | 5509 | O | VAL | B | 342 | 41.113 | 10.495 | 20.797 | 1.00 | 53.38 | O |
| ATOM | 5510 | N | ILE | B | 343 | 39.923 | 8.547 | 21.055 | 1.00 | 56.52 | N |
| ATOM | 5511 | CA | ILE | B | 343 | 40.061 | 7.995 | 19.681 | 1.00 | 51.53 | C |
| ATOM | 5512 | CB | ILE | B | 343 | 40.769 | 6.629 | 19.725 | 1.00 | 58.13 | C |
| ATOM | 5513 | CG1 | ILE | B | 343 | 42.208 | 6.783 | 20.238 | 1.00 | 65.86 | C |
| ATOM | 5514 | CG2 | ILE | B | 343 | 40.707 | 5.950 | 18.366 | 1.00 | 58.37 | C |
| ATOM | 5515 | CD1 | ILE | B | 343 | 43.020 | 5.506 | 20.291 | 1.00 | 69.59 | C |
| ATOM | 5516 | C | ILE | B | 343 | 38.655 | 7.943 | 19.091 | 1.00 | 47.07 | C |
| ATOM | 5517 | O | ILE | B | 343 | 37.855 | 7.110 | 19.545 | 1.00 | 52.20 | O |
| ATOM | 5518 | N | LEU | B | 344 | 38.369 | 8.833 | 18.139 | 1.00 | 46.06 | N |
| ATOM | 5519 | CA | LEU | B | 344 | 37.000 | 9.098 | 17.629 | 1.00 | 45.21 | C |
| ATOM | 5520 | CB | LEU | B | 344 | 36.961 | 10.359 | 16.757 | 1.00 | 43.62 | C |
| ATOM | 5521 | CG | LEU | B | 344 | 37.338 | 11.670 | 17.445 | 1.00 | 47.04 | C |
| ATOM | 5522 | CD1 | LEU | B | 344 | 37.272 | 12.829 | 16.455 | 1.00 | 47.76 | C |
| ATOM | 5523 | CD2 | LEU | B | 344 | 36.449 | 11.939 | 18.653 | 1.00 | 45.26 | C |
| ATOM | 5524 | C | LEU | B | 344 | 36.542 | 7.899 | 16.816 | 1.00 | 49.85 | C |
| ATOM | 5525 | O | LEU | B | 344 | 35.303 | 7.736 | 16.661 | 1.00 | 48.32 | O |
| ATOM | 5526 | N | GLU | B | 345 | 37.498 | 7.140 | 16.269 | 1.00 | 56.56 | N |
| ATOM | 5527 | CA | GLU | B | 345 | 37.192 | 5.904 | 15.501 | 1.00 | 65.62 | C |
| ATOM | 5528 | CB | GLU | B | 345 | 38.471 | 5.237 | 14.989 | 1.00 | 68.00 | C |
| ATOM | 5529 | CG | GLU | B | 345 | 39.084 | 5.957 | 13.795 | 1.00 | 70.33 | C |
| ATOM | 5530 | CD | GLU | B | 345 | 39.683 | 7.331 | 14.078 | 1.00 | 73.93 | C |
| ATOM | 5531 | OE1 | GLU | B | 345 | 39.837 | 7.680 | 15.286 | 1.00 | 71.04 | O |
| ATOM | 5532 | OE2 | GLU | B | 345 | 39.995 | 8.053 | 13.095 | 1.00 | 65.28 | O |
| ATOM | 5533 | C | GLU | B | 345 | 36.365 | 5.017 | 16.431 | 1.00 | 64.73 | C |
| ATOM | 5534 | O | GLU | B | 345 | 35.248 | 4.609 | 16.031 | 1.00 | 68.33 | O |
| ATOM | 5535 | N | ASN | B | 346 | 36.836 | 4.870 | 17.673 | 1.00 | 63.61 | N |
| ATOM | 5536 | CA | ASN | B | 346 | 36.211 | 4.000 | 18.702 | 1.00 | 63.03 | C |
| ATOM | 5537 | CB | ASN | B | 346 | 37.136 | 3.819 | 19.902 | 1.00 | 61.51 | C |
| ATOM | 5538 | CG | ASN | B | 346 | 38.439 | 3.147 | 19.516 | 1.00 | 60.57 | C |
| ATOM | 5539 | OD1 | ASN | B | 346 | 38.502 | 2.385 | 18.550 | 1.00 | 63.03 | O |
| ATOM | 5540 | ND2 | ASN | B | 346 | 39.494 | 3.425 | 20.261 | 1.00 | 59.91 | N |
| ATOM | 5541 | C | ASN | B | 346 | 34.811 | 4.518 | 19.051 | 1.00 | 66.37 | C |
| ATOM | 5542 | O | ASN | B | 346 | 33.933 | 3.659 | 19.234 | 1.00 | 69.86 | O |
| ATOM | 5543 | N | LEU | B | 347 | 34.584 | 5.840 | 19.065 | 1.00 | 68.41 | N |
| ATOM | 5544 | CA | LEU | B | 347 | 33.266 | 6.444 | 19.433 | 1.00 | 70.25 | C |
| ATOM | 5545 | CB | LEU | B | 347 | 33.405 | 7.956 | 19.661 | 1.00 | 75.39 | C |
| ATOM | 5546 | CG | LEU | B | 347 | 34.049 | 8.408 | 20.973 | 1.00 | 73.73 | C |
| ATOM | 5547 | CD1 | LEU | B | 347 | 33.814 | 9.894 | 21.194 | 1.00 | 66.92 | C |
| ATOM | 5548 | CD2 | LEU | B | 347 | 33.522 | 7.611 | 22.159 | 1.00 | 72.60 | C |
| ATOM | 5549 | C | LEU | B | 347 | 32.196 | 6.194 | 18.361 | 1.00 | 70.55 | C |
| ATOM | 5550 | O | LEU | B | 347 | 31.047 | 6.062 | 18.763 | 1.00 | 75.85 | O |
| ATOM | 5551 | N | TYR | B | 348 | 32.533 | 6.191 | 17.063 | 1.00 | 77.19 | N |
| ATOM | 5552 | CA | TYR | B | 348 | 31.554 | 6.290 | 15.941 | 1.00 | 82.03 | C |
| ATOM | 5553 | CB | TYR | B | 348 | 32.114 | 7.221 | 14.858 | 1.00 | 80.71 | C |
| ATOM | 5554 | CG | TYR | B | 348 | 31.363 | 7.334 | 13.546 | 1.00 | 79.28 | C |
| ATOM | 5555 | CD1 | TYR | B | 348 | 30.388 | 8.301 | 13.339 | 1.00 | 70.96 | C |
| ATOM | 5556 | CE1 | TYR | B | 348 | 29.755 | 8.443 | 12.112 | 1.00 | 67.86 | C |
| ATOM | 5557 | CZ | TYR | B | 348 | 30.093 | 7.613 | 11.056 | 1.00 | 72.52 | C |
| ATOM | 5558 | OH | TYR | B | 348 | 29.472 | 7.702 | 9.839 | 1.00 | 69.74 | O |
| ATOM | 5559 | CE2 | TYR | B | 348 | 31.073 | 6.656 | 11.238 | 1.00 | 70.98 | C |
| ATOM | 5560 | CD2 | TYR | B | 348 | 31.706 | 6.537 | 12.462 | 1.00 | 73.67 | C |
| ATOM | 5561 | C | TYR | B | 348 | 31.152 | 4.888 | 15.441 | 1.00 | 87.73 | C |
| ATOM | 5562 | O | TYR | B | 348 | 30.242 | 4.817 | 14.592 | 1.00 | 84.05 | O |
| ATOM | 5563 | N | PHE | B | 349 | 31.762 | 3.812 | 15.962 | 1.00 | 104.69 | N |
| ATOM | 5564 | CA | PHE | B | 349 | 31.243 | 2.417 | 15.843 | 1.00 | 115.71 | C |
| ATOM | 5565 | CB | PHE | B | 349 | 32.141 | 1.391 | 16.547 | 1.00 | 123.61 | C |
| ATOM | 5566 | CG | PHE | B | 349 | 31.677 | −0.048 | 16.445 | 1.00 | 144.81 | C |
| ATOM | 5567 | CD1 | PHE | B | 349 | 32.066 | −0.845 | 15.375 | 1.00 | 144.06 | C |
| ATOM | 5568 | CE1 | PHE | B | 349 | 31.648 | −2.167 | 15.277 | 1.00 | 138.81 | C |
| ATOM | 5569 | CZ | PHE | B | 349 | 30.833 | −2.712 | 16.244 | 1.00 | 136.44 | C |
| ATOM | 5570 | CD2 | PHE | B | 349 | 30.861 | −0.620 | 17.418 | 1.00 | 152.42 | C |
| ATOM | 5571 | CE2 | PHE | B | 349 | 30.437 | −1.939 | 17.313 | 1.00 | 144.67 | C |
| ATOM | 5572 | C | PHE | B | 349 | 29.829 | 2.365 | 16.440 | 1.00 | 111.86 | C |
| ATOM | 5573 | O | PHE | B | 349 | 28.816 | 2.265 | 15.748 | 1.00 | 102.46 | O |
| TER | 5574 | | PHE | B | 349 | | | | | | |
| HETATM | 5575 | O | HOH | S | 1 | 9.136 | 27.489 | 10.250 | 1.00 | 29.63 | O |
| HETATM | 5576 | O | HOH | S | 2 | 26.319 | 1.931 | 43.224 | 1.00 | 33.51 | O |
| HETATM | 5577 | O | HOH | S | 3 | 12.959 | 19.866 | −2.704 | 1.00 | 41.77 | O |
| HETATM | 5578 | O | HOH | S | 4 | 16.408 | 16.052 | −8.586 | 1.00 | 33.23 | O |
| HETATM | 5579 | O | HOH | S | 5 | 33.382 | 2.067 | 44.128 | 1.00 | 40.93 | O |
| HETATM | 5580 | O | HOH | S | 6 | 5.912 | 3.713 | −1.998 | 1.00 | 34.73 | O |
| HETATM | 5581 | O | HOH | S | 7 | 25.424 | −7.431 | −10.862 | 1.00 | 45.13 | O |
| HETATM | 5582 | O | HOH | S | 8 | 16.030 | −0.161 | −7.523 | 1.00 | 33.71 | O |
| HETATM | 5583 | O | HOH | S | 10 | 13.822 | 4.586 | −8.281 | 1.00 | 42.77 | O |
| HETATM | 5584 | O | HOH | S | 11 | 33.047 | 16.601 | 22.026 | 1.00 | 63.06 | O |
| HETATM | 5585 | O | HOH | S | 12 | 22.581 | 10.103 | 45.504 | 1.00 | 47.56 | O |
| HETATM | 5586 | O | HOH | S | 13 | 21.845 | 12.751 | −6.607 | 1.00 | 39.77 | O |
| HETATM | 5587 | O | HOH | S | 14 | 6.450 | 28.231 | 9.090 | 1.00 | 35.84 | O |
| HETATM | 5588 | O | HOH | S | 15 | 13.511 | −8.065 | −3.449 | 1.00 | 55.83 | O |
| HETATM | 5589 | O | HOH | S | 16 | 4.521 | 19.719 | 1.048 | 1.00 | 37.77 | O |
| HETATM | 5590 | O | HOH | S | 17 | 38.701 | −0.963 | 27.898 | 1.00 | 47.95 | O |
| HETATM | 5591 | O | HOH | S | 19 | 10.595 | 19.751 | 18.584 | 1.00 | 39.34 | O |
| HETATM | 5592 | O | HOH | S | 20 | 1.503 | 30.701 | 0.687 | 1.00 | 34.99 | O |
| HETATM | 5593 | O | HOH | S | 21 | 0.018 | 31.542 | −5.198 | 1.00 | 42.22 | O |
| HETATM | 5594 | O | HOH | S | 23 | 28.344 | 22.749 | 4.607 | 1.00 | 46.47 | O |
| HETATM | 5595 | O | HOH | S | 24 | 10.492 | 32.137 | −10.864 | 1.00 | 53.84 | O |
| HETATM | 5596 | O | HOH | S | 25 | 13.755 | 36.100 | −4.997 | 1.00 | 40.75 | O |
| HETATM | 5597 | O | HOH | S | 26 | 20.232 | −14.150 | 29.024 | 1.00 | 43.55 | O |
| HETATM | 5598 | O | HOH | S | 27 | 29.368 | 14.219 | 4.382 | 1.00 | 51.60 | O |
| HETATM | 5599 | O | HOH | S | 28 | −0.084 | 17.391 | 17.165 | 1.00 | 43.95 | O |
| HETATM | 5600 | O | HOH | S | 29 | 3.690 | 38.315 | 23.333 | 1.00 | 59.64 | O |
| HETATM | 5601 | O | HOH | S | 30 | 35.518 | −6.777 | 0.866 | 1.00 | 51.62 | O |
| HETATM | 5602 | O | HOH | S | 31 | 45.881 | 28.326 | 24.819 | 1.00 | 57.18 | O |
| HETATM | 5603 | O | HOH | S | 32 | 2.731 | 15.565 | −3.967 | 1.00 | 42.07 | O |
| HETATM | 5604 | O | HOH | S | 33 | 32.320 | 24.645 | 6.043 | 1.00 | 47.84 | O |
| HETATM | 5605 | O | HOH | S | 34 | 4.877 | 24.786 | −5.032 | 1.00 | 31.92 | O |
| HETATM | 5606 | O | HOH | S | 35 | 32.177 | −3.651 | 48.693 | 1.00 | 42.55 | O |
| HETATM | 5607 | O | HOH | S | 36 | 32.699 | 18.917 | −0.777 | 1.00 | 44.64 | O |
| HETATM | 5608 | O | HOH | S | 37 | 37.397 | 6.601 | 50.829 | 1.00 | 54.46 | O |
| HETATM | 5609 | O | HOH | S | 38 | 40.264 | −2.950 | 24.724 | 1.00 | 56.51 | O |
| HETATM | 5610 | O | HOH | S | 40 | 29.662 | −2.909 | 51.968 | 1.00 | 41.83 | O |
| HETATM | 5611 | O | HOH | S | 42 | 27.123 | −9.326 | −14.360 | 1.00 | 42.70 | O |
| HETATM | 5612 | O | HOH | S | 43 | 6.057 | 25.161 | −7.388 | 1.00 | 37.53 | O |
| HETATM | 5613 | O | HOH | S | 44 | 42.730 | 9.140 | 40.452 | 1.00 | 43.74 | O |
| HETATM | 5614 | O | HOH | S | 45 | 20.601 | −6.950 | −16.794 | 1.00 | 65.21 | O |
| HETATM | 5615 | O | HOH | S | 46 | −1.457 | 26.456 | 9.349 | 1.00 | 42.13 | O |
| HETATM | 5616 | O | HOH | S | 47 | −1.756 | 26.505 | 13.483 | 1.00 | 56.33 | O |
| HETATM | 5617 | O | HOH | S | 48 | 32.786 | −14.885 | 5.206 | 1.00 | 54.38 | O |
| HETATM | 5618 | O | HOH | S | 49 | 35.330 | 18.745 | 19.870 | 1.00 | 66.91 | O |
| HETATM | 5619 | O | HOH | S | 50 | 24.755 | 6.794 | 1.064 | 1.00 | 47.91 | O |
| HETATM | 5620 | O | HOH | S | 51 | 5.402 | 17.178 | −11.448 | 1.00 | 75.84 | O |
| HETATM | 5621 | O | HOH | S | 52 | 22.114 | −5.868 | 38.470 | 1.00 | 45.69 | O |
| HETATM | 5622 | O | HOH | S | 53 | 4.606 | 11.717 | 4.948 | 1.00 | 41.72 | O |
| HETATM | 5623 | O | HOH | S | 54 | 18.470 | 12.232 | 17.753 | 1.00 | 44.96 | O |
| HETATM | 5624 | O | HIOH | S | 55 | 13.155 | 15.889 | −9.101 | 1.00 | 42.30 | O |
| HETATM | 5625 | O | HOH | S | 56 | 28.689 | 18.482 | 49.256 | 1.00 | 56.09 | O |
| HETATM | 5626 | O | HOH | S | 57 | 38.910 | 5.826 | 48.538 | 1.00 | 40.09 | O |
| HETATM | 5627 | O | HOH | S | 58 | 21.485 | 8.212 | −1.948 | 1.00 | 43.78 | O |
| HETATM | 5628 | O | HOH | S | 59 | 10.405 | 4.489 | 40.606 | 1.00 | 52.10 | O |
| HETATM | 5629 | O | HOH | S | 60 | 39.946 | 30.291 | 40.996 | 1.00 | 44.88 | O |
| HETATM | 5630 | O | HOH | S | 61 | 9.342 | 6.517 | 41.142 | 1.00 | 46.99 | O |
| HETATM | 5631 | O | HOH | S | 62 | 20.675 | −1.687 | 4.811 | 1.00 | 43.51 | O |
| HETATM | 5632 | O | HOH | S | 63 | 11.444 | 36.828 | −8.112 | 1.00 | 61.12 | O |
| HETATM | 5633 | O | HOH | S | 64 | 19.765 | 13.766 | −12.814 | 1.00 | 46.56 | O |
| HETATM | 5634 | O | HOH | S | 65 | 24.416 | −7.526 | 31.004 | 1.00 | 52.38 | O |
| HETATM | 5635 | O | HOH | S | 66 | 6.607 | 3.059 | −4.232 | 1.00 | 43.66 | O |
| HETATM | 5636 | O | HOH | S | 67 | 5.698 | 5.580 | 15.996 | 1.00 | 53.55 | O |
| HETATM | 5637 | O | HOH | S | 68 | 21.977 | 16.620 | 21.036 | 1.00 | 59.19 | O |
| HETATM | 5638 | O | HOH | S | 69 | 46.029 | 8.231 | 46.253 | 1.00 | 56.16 | O |
| HETATM | 5639 | O | HOH | S | 70 | 13.680 | −7.247 | 45.302 | 1.00 | 48.06 | O |
| HETATM | 5640 | O | HOH | S | 71 | 43.320 | 6.333 | 40.084 | 1.00 | 59.98 | O |
| HETATM | 5641 | O | HOH | S | 72 | 22.236 | 21.752 | −2.365 | 1.00 | 52.89 | O |
| HETATM | 5642 | O | HOH | S | 73 | 51.863 | 4.656 | 48.700 | 1.00 | 63.51 | O |
| HETATM | 5643 | O | HOH | S | 74 | 17.142 | 8.599 | 51.414 | 1.00 | 48.10 | O |
| HETATM | 5644 | O | HOH | S | 75 | 9.291 | −10.366 | 42.758 | 1.00 | 56.19 | O |
| HETATM | 5645 | O | HOH | S | 76 | 21.152 | −13.030 | −14.753 | 1.00 | 55.91 | O |
| HETATM | 5646 | O | HOH | S | 77 | 9.608 | 0.369 | −7.009 | 1.00 | 43.56 | O |
| HETATM | 5647 | O | HOH | S | 78 | 17.614 | 4.623 | 3.788 | 1.00 | 65.39 | O |
| HETATM | 5648 | O | HOH | S | 79 | 20.279 | 0.534 | −20.169 | 1.00 | 50.60 | O |
| HETATM | 5649 | O | HOH | S | 80 | 19.712 | 21.602 | −9.017 | 1.00 | 67.89 | O |
| HETATM | 5650 | O | HOH | S | 81 | 35.371 | 36.351 | 41.218 | 1.00 | 53.32 | O |
| HETATM | 5651 | O | HOH | S | 82 | 38.536 | 13.595 | 4.793 | 1.00 | 53.15 | O |
| HETATM | 5652 | O | HOH | S | 83 | 17.738 | 0.675 | −20.722 | 1.00 | 57.15 | O |
| HETATM | 5653 | O | HOH | S | 84 | 8.617 | 25.689 | −7.257 | 1.00 | 43.28 | O |
| HETATM | 5654 | O | HOH | S | 85 | 17.967 | 5.041 | −22.143 | 1.00 | 53.00 | O |
| HETATM | 5655 | O | HOH | S | 86 | 6.353 | 11.547 | −5.692 | 1.00 | 36.77 | O |
| HETATM | 5656 | O | HOH | S | 87 | 31.360 | 10.434 | 51.348 | 1.00 | 43.17 | O |
| HETATM | 5657 | O | HOH | S | 88 | 54.307 | 8.399 | 25.162 | 1.00 | 53.26 | O |
| HETATM | 5658 | O | HOH | S | 89 | 29.910 | 0.093 | 56.446 | 1.00 | 55.24 | O |
| HETATM | 5659 | O | HOH | S | 90 | 19.725 | −8.368 | −5.107 | 1.00 | 34.85 | O |
| HETATM | 5660 | O | HOH | S | 91 | −0.847 | 28.967 | 14.765 | 1.00 | 55.49 | O |
| HETATM | 5661 | O | HOH | S | 92 | −1.838 | 17.239 | −4.352 | 1.00 | 49.15 | O |
| HETATM | 5662 | O | HOH | S | 93 | 6.634 | 14.288 | −9.266 | 1.00 | 58.40 | O |
| HETATM | 5663 | O | HOH | S | 94 | 23.520 | 22.395 | −7.691 | 1.00 | 43.52 | O |
| HETATM | 5664 | O | HOH | S | 95 | 1.266 | 24.146 | 23.898 | 1.00 | 51.43 | O |
| HETATM | 5665 | O | HOH | S | 96 | 17.119 | −8.182 | −5.254 | 1.00 | 36.55 | O |
| HETATM | 5666 | O | HOH | S | 97 | 11.249 | 0.846 | −12.206 | 1.00 | 59.81 | O |
| HETATM | 5667 | O | HOH | S | 98 | 16.411 | −18.410 | −13.019 | 1.00 | 58.37 | O |

1. A crystal comprising a calpain-5 protease core domain having a G267S mutation, wherein the crystal has P12₁1 space group symmetry and a unit cell having dimensions of a=84.0 Å, b=51.6 Å, c=110.9 Å, α=90°, β=110.4°, and γ=90°.

2. The crystal of claim 1, wherein the calpain-5 protease core domain having the G267S mutation comprises or consists of the amino acid sequence of SEQ ID NO:1 or an amino acid sequence having at least 95% identity to the sequence of SEQ ID NO:1.

3. The crystal of claim 1, wherein X-ray diffraction data collected from the crystal can be used to determine a structure of the calpain-5 protease core domain having the G267S mutation comprising atomic coordinates listed in Table 2±a root mean square deviation of less than 2 Å.

4. The crystal of claim 1, wherein the crystal is obtainable by crystallization of the calpain-5 protease core domain having the G267S mutation in a solution comprising or consisting of about 9% to about 11% polyethylene glycol (PEG) 8000 and a buffer at a pH of about 5.5.

5. The crystal of claim 4, wherein the buffer is 100 mM sodium citrate dihydrate.

6. A composition comprising the crystal of claim 1.

7. The composition of claim 6, wherein the crystal diffracts x-rays to allow determination of structure coordinates to a resolution of 2.2 Å.

8. A method of producing the crystal of claim 1, the method comprising crystallizing the calpain-5 protease core domain having the G267S mutation in a crystallization solution comprising or consisting of about 9% to about 11% polyethylene glycol (PEG) 8000 and a buffer at a pH of about 5.5.

9. The method of claim 8, wherein the buffer is 100 mM sodium citrate dihydrate.

10. The method of claim 8, further comprising soaking the crystal in a solution comprising an inhibitor or a substrate of calpain-5 such that the inhibitor or substrate binds to the active site of the calpain-5 protease core domain having the G267S mutation within the crystal.

11. The method of claim 8, wherein the crystallization solution further comprises an inhibitor or a substrate of calpain-5.

12. The crystallographic structure of the crystal of claim 1 having the atomic coordinates listed in Table 2.

13. (canceled)

14. A method for identifying a small molecule that binds to the calpain-5 protease core domain having the G267S mutation and inhibits calpain-5 protease activity, the method comprising:
a) screening in silico a small molecule library for candidate small molecules likely to bind to the calpain-5 protease core domain using a three-dimensional model of the calpain-5 protease core domain that is computationally derived from the atomic coordinates of the crystallographic structure of claim 12; and
b) evaluating the candidate small molecules identified in step (a) as likely to bind to the calpain-5 protease core domain for their ability to inhibit the calpain-5 variant having a G267S mutation using one or more in vitro or in vivo assays to identify at least one candidate small molecule that inhibits calpain-5 protease activity.

15. The method of claim 14, wherein in step (a), the small molecule library is screened using computational docking for the candidate small molecules, wherein a docking score is calculated for docking of each candidate small molecule in the three-dimensional model of the protease core.

16. A computer readable medium comprising the atomic coordinates listed in Table 2.

17. A method for designing an inhibitor of calpain 5, the method comprising:
a) obtaining a crystal comprising a calpain-5 protease core domain having a G267S mutation, wherein the crystal has P12₁1 space group symmetry and a unit cell having dimensions of a=84.0 Å, b=51.6 Å, c=110.9 Å, a=90°, b=110.4°, and g=90′;
b) determining the three-dimensional structure of the calpain-5 protease core domain having the G267S mutation using the crystal obtained in (a) by X-ray crystallography to obtain atomic coordinates of the structure;
c) providing the atomic coordinates of the three-dimensional structure of the calpain-5 protease core domain having the G267S mutation on a computer; and
d) utilizing a program operated by the computer to design a chemical compound predicted to bind to the calpain-5 protease core domain having the G267S mutation at a binding location and inhibit protease activity of calpain-5.

18. The method of claim 17, wherein the designing involves de novo rational drug design.

19. The method of claim 18, wherein the rational drug design involves (i) identification of functional groups and/or small molecule fragments which can interact with sites in the binding location within the calpain-5 protease core domain, and (ii) linking the functional groups and/or small molecule fragments in a single compound.

20. The method of claim 18, wherein the designing involves utilizing docking software and screening one or more databases for molecules that fit the binding location within the protease core domain of calpain-5.

21. The method of claim 17, further comprising:
obtaining the compound; and
evaluating the compound for (1) binding to calpain-5, (2) competing with a substrate of calpain-5 for binding to the substrate binding site within the calpain-5 protease core domain, or (3) inhibiting protease activity of calpain-5, or any combination thereof.

22. The method of claim 17, wherein the binding location is in a substrate binding pocket or the active site within the protease core domain of calpain-5.

23. The method of claim 22, wherein the compound binds to the S1 sub-pocket, S2 sub-pocket, S3 sub-pocket, or S4 sub-pocket of the substrate binding site, or any combination thereof.

24. (canceled)

25. The method of claim 17, wherein the rational drug design is based on an interaction between the compound and a residue of the G1 loop, G2 loop, or PC2L2 loop of calpain-5.

26. The method of claim 17, wherein the residue is at amino acid position 81, 243, 244, 250, 252, 267, 284, or 289 numbered relative to the reference sequence of SEQ ID NO:1.

27. A computer system comprising the atomic coordinates listed in Table 2 stored in memory.


SEQUENCE LISTING (1 sequence)

SEQ ID NO: 1 - protein, 343 aa, Artificial Sequence
  mutant human G267S calpain-5 protease core domain
MFSCVKPYEDQNYSALRRDCRRRKVLFEDPLFPATDDSLYYKGTPGPAVRWKRPKGICEDPRLFVDGISSHDLHQGQVGNCWFVAACSSLASRESLWQKVIPDWKEQEWDPEKPNAYAGIFHFHFWRFGEWVDVVIDDRLPTVNNQLIYCHSNSRNEFWCALVEKAYAKLAGCYQALDGGNTADALVDFTGGVSEPIDLTEGDFANDETKRNQLFERMLKVHSRGGLISASIKAVTAADMEARLACGLVKGHAYAVTDVRKVRLGHSLLAFFKSEKLDMIRLRNPWGEREWNGPWSDTSEEWQKVSKSEREKMGVTVQDDGEFWMTFEDVCRYFTDIIKCRVI